(19) United States
(12) Patent Application Publication
(10) Pub. No.: US 2025/0230147 A1
(43) Pub. Date: Jul. 17, 2025

(54) LACTAMS AS CBL-B INHIBITORS

(71) Applicant: Genentech, Inc., South San Francisco, CA (US)

(72) Inventors: Jun Liang, Los Altos, CA (US); Araz Jakalian, Montreal (CA); Michael John Lambrecht, Burlingame, CA (US); Robin Larouche-Gauthier, Montreal (CA); Malcolm Huestis, San Francisco, CA (US); Man Un Ung, San Mateo, CA (US); Xiaojing Wang, Belmont, CA (US); Arun Yadav, Montreal (CA); Jason Robert Zbieg, Montara, CA (US); Fabio Broccatelli, Redwood City, CA (US)

(73) Assignee: Genentech, Inc., South San Francisco, CA (US)

(21) Appl. No.: 18/939,425

(22) Filed: Nov. 6, 2024

Related U.S. Application Data
(63) Continuation of application No. 17/592,422, filed on Feb. 3, 2022, now abandoned.
(60) Provisional application No. 63/145,401, filed on Feb. 3, 2021.

(51) Int. Cl. C07D 405/14; A61P 35/00; C07D 401/14; C07D 403/10; C07D 403/14; C07D 409/14; C07D 491/107

(52) U.S. Cl. CPC C07D 405/14; A61P 35/00; C07D 401/14; C07D 403/10; C07D 403/14; C07D 409/14; C07D 491/107

(57) ABSTRACT

Various lactam compound that binds Cbl-B, many of which are selective for Cbl-B over C-Cbl, and methods of making and using the same. Representative lactam compounds include molecules falling within the following formulae:

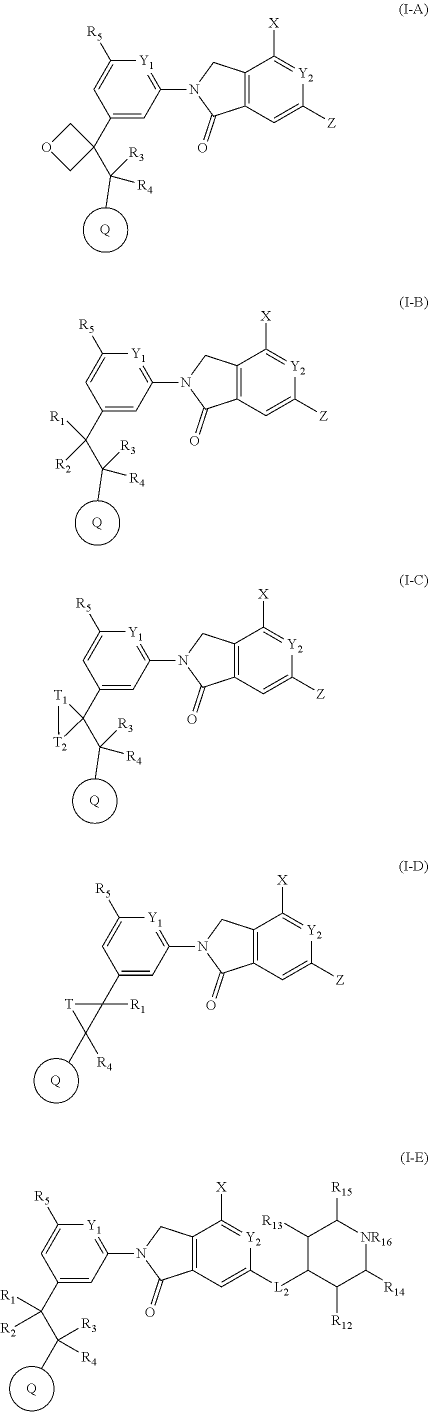

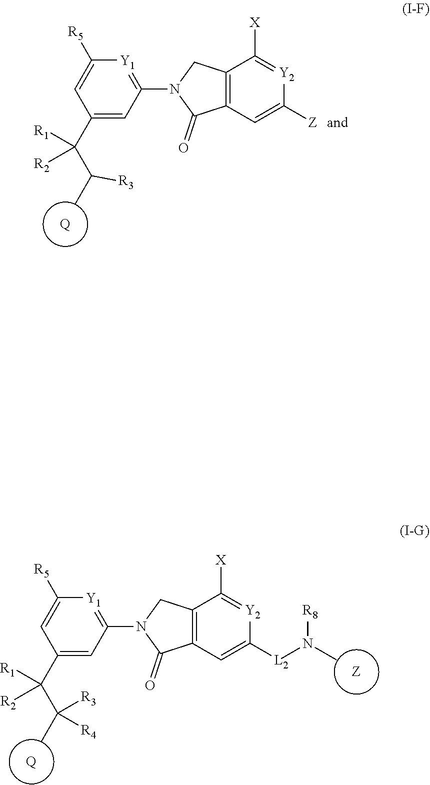

### CLAIM OF PRIORITY

This application claims benefit of priority to U.S. provisional patent application No. 63/145,401, filed Feb. 3, 2021, the contents of which are incorporated by reference herein.

### TECHNICAL FIELD

The technology described herein generally relates to inhibitors of Cbl-B and which also have activity against, or which can be selective over, C-Cbl, and additionally relates to methods of making and using the same.

### BACKGROUND

Casitas B-lineage lymphoma-b (Cbl-b) is a member of the Cbl family of RING E3 ubiquitin ligases. A common function of Cbl family proteins is the negative regulation of receptor tyrosine kinase signaling. Since Cbl-b inhibition leads to immune activation, it has been expected that Cbl-b inhibitors could be broadly active in multiple oncology indications.

Cbl proteins comprise three principal domains: a conserved N-terminal tyrosine kinase binding (TKB) domain, a short linker region, and a RING finger (RF) domain. The TKB domain is, in turn, composed of three subdomains: a 4-helix bundle (4H), a calcium-binding domain with an EF-hand fold, and a variant Src homology region 2 (SH2) domain, all three of which are involved in phosphotyrosine binding. The TKB domain binds substrates, such as ZAP70, that contain phosphotyrosine motifs.

The conserved RF domain, which has intrinsic E3 ligase activity, can recruit E2 ubiquitin-conjugating enzymes, and mediate the transfer of ubiquitin to substrates.

Phosphorylation of Cbl-b at Y₃₆₃ within the linker domain regulates its E3 ubiquitin ligase activity by removing the masking of the RF domain by the TKB domain.

In T cells, Cbl-b is a key tolerogenic factor that directly regulates the cells' activation. Specifically, Cbl-b is highly expressed in murine and human CD4+ and CD8+T cells, where it functions as a potent negative regulator of T cell activation by controlling activation thresholds and the requirement for co-stimulation. Mechanistically Cbl-b acts by ubiquitinating multiple substrates downstream of the T cell receptor (TCR), including ZAP70, resulting in TCR internalization and termination of signaling. Loss of Cbl-b in T cells leads to prolonged TCR surface expression, and in combination with TCR stimulation results in increased expression of activation markers, such as CD25, cytokine production and proliferation.

Mouse models have surprisingly demonstrated that the loss of Cbl-b leads to increased adaptive and innate anti-tumor immunity, mediated by enhanced T cell effector function as well as increased natural killer (NK) cell activity. Cbl-b deficient mice spontaneously reject a variety of cancer tumors, including spontaneous solid tumors and hematopoietic malignancies, in a CD8 T cell-dependent manner. Adoptive transfer of Cbl-b-/—CD8+ T cells is sufficient to reject tumors, demonstrating that Cbl-b has a non-redundant role in regulating T-cell-mediated anti-tumor activity.

Consequently, developing a small molecule approach to inhibit Cbl-b is a promising but challenging goal for cancer immunotherapy.

Nevertheless, c-Cbl, a closely related family member to Cbl-b, shares high sequence homology with Cbl-b at the N-terminus, including in the TKB and RING domains. c-Cbl negatively regulates signaling of a number of growth factor receptors, including Flt3 and c-Kit. Among other defects, c-Cbl deficient mice exhibit expansion of hematopoietic stem cells and multipotent progenitors in the bone marrow. In mice that are conditionally deficient in both c-Cbl and Cbl-b this defect is amplified, and the mice develop a rapidly-progressive and lethal myeloproliferative disease accompanied by splenomegaly by around 8 weeks of age. Given a broad spectrum of functions of c-Cbl in growth factor receptor regulation, and a strong amplification in the dysregulation of these pathways in the absence of both c-Cbl and Cbl-b, compounds with selectivity for Cbl-b over c-Cbl are likely to be highly desirable as cancer immunotherapy agents. Compounds having activity against both Cbl-b, and c-Cbl, which could be termed “pan-cbl”, may yet prove beneficial if their inhibitory effect against Cbl-b is potent enough.

Accordingly, there is a need for compounds that both inhibit Cbl-b and that either exhibit selectivity over binding to c-Cbl or have a known activity against c-Cbl.

The discussion of the background herein is included to explain the context of the technology. This is not to be taken as an admission that any of the material referred to was publicly available, known, or part of the common general knowledge as at the priority date of any of the claims found appended hereto.

Throughout the description and claims of the application the word “comprise” and variations thereof, such as “comprising” and “comprises”, is not intended to exclude other additives, components, integers or steps.

### SUMMARY

The instant disclosure addresses compounds for inhibiting the Cbl-B receptor that also exhibit inhibitory activity against, and in some cases are selective over, the C-Cbl receptor. In particular, the disclosure comprises a number of such compounds and methods for using the same.

The present disclosure provides for compounds of formulae (I-A), (I-B), (I-C), (I-D), (I-E), (I-F), and (I-G):

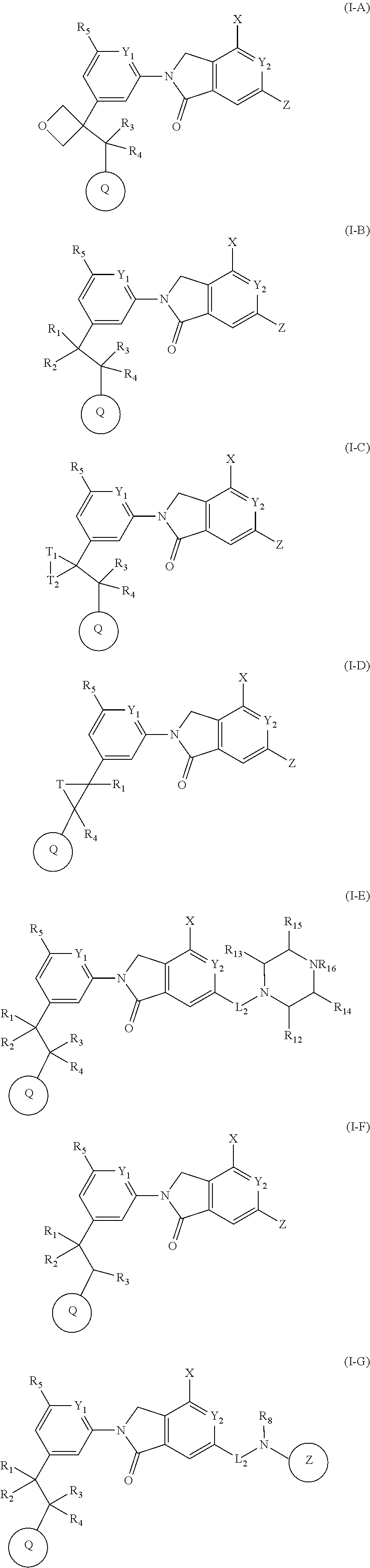

In formulae (I-A), (I-B), (I-C), (I-D), (I-E), (I-F), and (I-G), Q is a 5-membered heteroaryl group, optionally substituted by one or more alkyl, cycloalkyl, or haloalkyl groups.

The present disclosure includes a process for making compounds of formulae (I-A), (I-B), (I-C), (I-D), (I-E), (I-F) and (I-G).

The present disclosure further includes a method of treatment comprising administering a compound of formula (I-A), (I-B), (I-C), (I-D), (I-E), (I-F) and (I-G) optionally in combination with another agent, such as a checkpoint inhibitor, to a patient suffering from cancer.

### BRIEF DESCRIPTION OF THE DRAWINGS

FIG. 1 shows an exemplary synthetic scheme for compounds described herein;

FIG. 2 shows an exemplary synthetic scheme for compounds described herein;

FIG. 3 shows an exemplary synthetic scheme for compounds described herein;

FIG. 4 shows an exemplary synthetic scheme for compounds described herein;

FIGS. 5A-5N and FIGS. 5P-5X show exemplary synthetic schemes to intermediate compounds in synthesis of compounds disclosed herein;

FIGS. 6-48 show representative synthetic schemes to various exemplary compounds disclosed herein.

FIG. 49 shows a table of activity data for select compounds herein.

Like reference symbols in the various drawings indicate like elements.

### DETAILED DESCRIPTION

The instant disclosure is directed to compounds that bind to the Cbl-b inhibitor and that either exhibit selectivity over C-Cbl or have inhibitory activity against c-Cbl. Methods of making such compounds, as well as assays for assessing their potency and selectivity, as well as metabolic and permeability properties, are also described herein.

### Structures

The instant disclosure comprises compounds of formula (I-A),

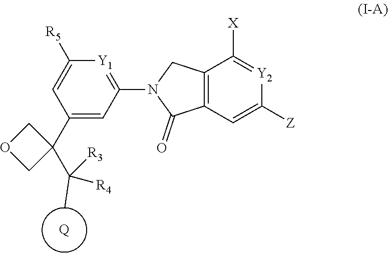

wherein:
Q is a 5-membered heteroaryl, optionally substituted by one or more alkyl, cycloalkyl, aryl, or haloalkyl groups;
Y₁ and Y₂ are independently CH, CF, or N;
R₃, R₄ are independently selected from: H, halogen, alkyl, CN, OH, alkoxy, and haloalkyl, wherein at least one of R₃ and R₄ is halogen;
R₅ is selected from: H, halo, CN, or L₁ₐ-R₁₀, wherein L₁ₐ is —C(L_{1b}R₁₁)(R₁₂)—, —N(L_{1b}R₁₁)—, —C(═O)N(L_{1b}R₁₁)—, O, S, carbonyl, or a bond, wherein:
   L_{1b} is alkylene or a bond; and
   R₁₀, R₁₁ and R₁₂ are independently H, alkyl, alkoxyalkyl, aminoalkyl, cycloalkyl, bridged bicyclyl, fused bicyclyl, spirocyclyl, alkenyl, cycloalkenyl, alkynyl, amidoalkyl, aryl, heteroaryl, or heterocyclyl, and R₁₀, R₁₁, and R₁₂ are each optionally substituted by one or more groups selected from: halo, CN, amino, alkylamino, alkyl carbonyl, OH, oxo, alkoxy, alkyl, cycloalkyl, haloalkyl, alkoxyalkyl, sulfonamidyl, alkyl sulfonamidyl, heterocyclyl, aryl, and heteroaryl;
X is halo, haloalkyl, or cycloalkyl; and
Z is —L₂NR₇R₈ or —C(H)(NR₇R₈)R₆ₐ;
wherein:
L₂ is —C(H)R₆ₐ—, —C(═O)—, or a bond;
R₆ₐ=H, alkyl, cycloalkyl, or haloalkyl; and
R₇ and R₈ are independently selected from H, alkyl, cycloalkyl, spirocyclyl, bridged bicyclyl, hydroxyalkyl, heterocyclyl, and haloalkyl,
wherein, if any of R₇ or R₈ is alkyl, cycloalkyl or heterocyclyl, said alkyl, cycloalkyl or heterocyclyl group is optionally substituted with one or more groups selected from: sulfonyl, halo, hydroxyl, alkoxy, alkyl, cycloalkyl, heterocyclyl, alkoxyalkyl, alkenyl, hydroxyalkyl, cyano, carboxylalkyl, and haloalkyl;
or
R₇ and R₈ together with the nitrogen atom to which they are both bonded form a:
   3-8 membered saturated monocyclic ring,
   wherein the saturated monocyclic ring is optionally substituted with one or more groups selected from: sulfonyl, halo, hydroxyl, alkoxy, alkyl, cycloalkyl, alkoxyalkyl, alkenyl, hydroxyalkyl, oxo, carboxylalkyl, and haloalkyl; with the provisos that:
      when Y₁ and Y₂ are both CH, R₅=H, X=CF₃, and Q is 2-methyl triazol-1-yl:
      if R₃=R₄=F, and L₂ is CH₂, the substituted saturated monocyclic ring is not 3-fluoro-azetidyn-1-yl;
      if R₃=R₄=F, and L₂ is CH(CH₃), the substituted saturated monocyclic ring is not 3-fluoro-pyrrolidin-1-yl, and
      if R₃ is F, R₄ is H, and L₂ is CH₂, the substituted saturated monocyclic ring is not one of: 4-fluorocyclohexamin-1-yl, cyclohexamin-1-yl, and pyrrolidin-1-yl;
or
R₇ and R₈ together with the nitrogen atom to which they are both bonded form a: 3-8 membered saturated spirocyclic ring,
   wherein the saturated spirocyclic ring is optionally substituted with one or more groups selected from: sulfonyl, halo, hydroxyl, cyano, alkyl, alkenyl, cycloalkyl, alkoxy, alkoxyalkyl, hydroxyalkyl, oxo, carboxylalkyl, and haloalkyl;
   with the proviso that:
   when Y₁ and Y₂ are both CH, R₃ is F and R₄ is H, R₅=H, L₂ is —C(H)R₆—, R₆ is H or methyl, X=CF₃, and Q is 2-methyl triazol-1-yl, the substituted saturated spirocyclic ring is not 5-azaspiro[2.4]hept-5-yl;
or
R₇ and R₈ together with the nitrogen atom to which they are both bonded form a: 3-8 membered saturated fused bicyclic ring, wherein the saturated fused bicyclic ring is optionally substituted with one or more groups selected from: sulfonyl, halo, hydroxyl, cyano, alkoxy, alkyl, cycloalkyl, alkoxyalkyl, alkenyl, hydroxyalkyl, oxo, carboxylalkyl, and haloalkyl;
or
R₇ and R₈ together with the nitrogen atom to which they are both bonded form a:
   3-8 membered saturated bridged bicyclic ring,
   wherein the saturated bridged bicyclic ring is optionally substituted with one or more groups selected from: sulfonyl, halo, hydroxyl, cyano, alkoxy, alkyl, cycloalkyl, alkoxyalkyl, alkenyl, hydroxyalkyl, oxo, carboxylalkyl, and haloalkyl; with the proviso that:
   when Y₁ and Y₂ are both CH, R₃ is F and R₄ is H, R₅=H, L₂ is CH₂, X=CF₃, and Q is 2-methyl triazol-1-yl, the substituted saturated bridged bicyclic ring is not 7-azabicyclo[2.2.1]hept-7-yl, 3-fluoro-8-azabicyclo[3.2.1]oct-8-yl; or 8-azabicyclo[3.2.1]oct-8-yl;
or Z is

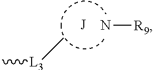

wherein:
   L₃ is —C(H)R_{6b}—, —N(R_{6b})—, O, —OC(H)(R_{6b})—, S, or a bond;
   R_{6b}=H, alkyl, cycloalkyl, or haloalkyl;
   J is a saturated 3-10 membered amine containing ring selected from a monocyclic ring, spirocyclic ring, bridged bicyclic ring, and a fused bicyclic ring, or a 5 or 6 member heteroaromatic ring, or a 3-10 member fused heteroaromatic ring system, and wherein:
   J is optionally substituted by one or more sulfonyl, halo, hydroxyl, cyano, alkoxy, alkyl, cycloalkyl, alkoxyalkyl, alkenyl, hydroxyalkyl, oxo, carboxyalkyl, or haloalkyl groups; and
   R₉=H, alkyl, aminoalkyl, haloalkyl, or carboxyalkyl;
else
Z is H,
or an enantiomer, diastereomer or a pharmaceutically acceptable salt or solvate thereof.

In some embodiments, the compound has formula (I-A), wherein R₃ is F and R₄ is H.

In some embodiments, the compound has formula (I-A), wherein R₅ is H.

In some embodiments, the compound has formula (I-A), wherein Y₁=Y₂=CH.

In some embodiments, the compound has formula (I-A), wherein X=CF₃.

In some embodiments, the compound has formula (I-A), wherein Q is 2-methyl triazol-1-yl or imidazolyl.

In some embodiments, the compound has formula (I-A), wherein Z is —C(H)R₆NR₇R₈ and R₆ is H or methyl.

In some embodiments, the compound has formula (I-A), wherein Z is —C(H)R₆NR₇R₈ and R₇ and R₈ together with the nitrogen atom to which they are both bonded form a saturated monocyclic ring selected from: azetidin-1-yl, pyrrolidin-1-yl, piperazine-1-yl, piperidin-1-yl, morpholin-4-yl, hexahydropyrimidin-1-yl, and 1,4-oxazepan-4-yl.

In some embodiments, the compound has formula (I-A), wherein Z is —L₂NR₇R₈, and L₂ is CH₂ or C(H)Me, and R₇ and R₈ and the nitrogen to which they are both bonded form a piperazin-1-yl ring.

In some embodiments, the compound has formula (I-A), wherein Z is —L₂NR₇R₈, and L₂ is CH₂ or C(H)Me, and R₇ and R₈ and the nitrogen to which they are both bonded form a piperazin-1-yl ring substituted with one or more groups selected from: alkyl, sulfonyl, acetyl, haloalkyl, cycloalkyl, and oxetanyl.

In some embodiments, the compound has formula (I-A), wherein R₇ and R₈ together with the nitrogen atom to which they are both bonded form a 3-8 membered saturated monocyclic ring that is optionally substituted with one or more groups selected from: methyl, fluoromethyl, hydroxyethyl, chloromethyl, hydroxyl, propyl, isopropyl, methoxy, methoxymethyl, difluoromethyl, methoxyethyl, vinyl, methylsulfonyl, 2-fluoroethyl, acetyl, and 1,1,1-trifluroethyl.

In some embodiments, the compound has formula (I-A), wherein Z is —C(H)R₆NR₇R₈ and R₇ and R₈ together with the nitrogen atom to which they are both bonded form an azetidin-1-yl ring that is optionally substituted with one or more groups selected from: methyl, ethyl, propyl, isopropyl, 2-hydroxyethyl, fluoromethyl, chloromethyl, hydroxymethyl, methoxy, vinyl, hydroxyl, methoxymethyl, and difluoromethyl.

In some embodiments, the compound has formula (I-A), wherein the azetidin-1-yl ring is substituted at the 3-position with two groups selected from methyl, ethyl, propyl, isopropyl, 2-hydroxyethyl, fluoromethyl, chloromethyl, hydroxymethyl, methoxy, vinyl, hydroxyl, methoxymethyl, and difluoromethyl.

In some embodiments, the compound has formula (I-A), wherein Z is —C(H)R₆NR₇R₈ and R₇ and R₈ together with the nitrogen atom to which they are both bonded form a spirocyclic ring selected from: 6-oxa-1-azaspiro[3.3]heptan-1-yl, 2-azaspiro[3.3]heptan-2-yl, 5-azaspiro[2.3]hexane, 1,1-difluoro-5-azaspiro[2.3]hex-5-yl, and 6-thia-1-azaspiro[3.3]hept-1-yl-6,6-dioxide.

In some embodiments, the compound has formula (I-A), wherein Z is —C(H)R₆NR₇R₈ and R₇ and R₈ together with the nitrogen atom to which they are both bonded form a fused bicyclic ring that is 3-azabicyclo[3.1.0]hexan-3-yl.

In some embodiments, the compound has formula (I-A), wherein Z is —C(H)R₆NR₇R₈ and R₇ and R₈ together with the nitrogen atom to which they are both bonded form a bridged bicyclic ring that is 2-azabicyclo[2.1.1]hex-2-yl.

In some embodiments, the compound has formula (I-A), wherein Z is —C(H)R₆NR₇R₈ and R₆ is cyclopropyl.

In some embodiments, the compound has formula (I-A), wherein Z is

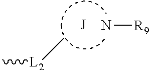

and L₂ is methylene.

The compound of claim 1, wherein Z is

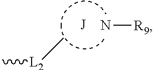

L₂ is O, J is 1H-pyrazol-4-yl, and R₉=H.

In some embodiments, the compound has formula (I-A), wherein Z is

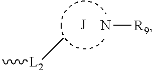

and J is 3-azetidynyl.

In some embodiments, the compound has formula (I-A), wherein Z is H.

In some embodiments, the compound has formula (I-A), wherein R₇ and R₈ together with the nitrogen atom to which they are both bonded form an azetidin-1-yl ring that is optionally substituted with one or more groups selected from: methyl, ethyl, propyl, isopropyl, 2-hydroxyethyl, fluoromethyl, chloromethyl, hydroxymethyl, methoxy, vinyl, hydroxyl, methoxymethyl, and difluoromethyl. In still further embodiments, the azetidin-1-yl ring is substituted at the 3-position with two groups selected from methyl, ethyl, propyl, isopropyl, 2-hydroxyethyl, fluoromethyl, chloromethyl, hydroxymethyl, methoxy, vinyl, hydroxyl, methoxymethyl, and difluoromethyl.

In some embodiments, the compound has formula (I-A), wherein R₇ and R₈ together with the nitrogen atom to which they are both bonded form a spirocyclic ring selected from: 6-oxa-1-azaspiro[3.3]heptan-1-yl, 2-azaspiro[3.3]heptan-2-yl, 5-azaspiro[2.3]hexane, 1,1-difluoro-5-azaspiro[2.3]hex-5-yl, and 6-thia-1-azaspiro[3.3]hept-1-yl-6,6-dioxide.

In some embodiments, the compound has formula (I-A), wherein R₇ and R₈ together with the nitrogen atom to which they are both bonded form a fused bicyclic ring that is 3-azabicyclo[3.1.0]hexan-3-yl.

In some embodiments, the compound has formula (I-A), wherein R₇ and R₈ together with the nitrogen atom to which they are both bonded form a bridged bicyclic ring that is 2-azabicyclo[2.1.1]hex-2-yl.

In some embodiments, the compound has formula (I-A), wherein Z is —CH₂NHR₇, wherein R₇ is cycloalkyl substituted by an alkyl group.

In some embodiments, the compound has formula (I-A), wherein R₇ is 1-methyl-cyclobut-1-yl.

In some embodiments, the compound has formula (I-A), wherein R₅ is L₁ₐ-R₁₀.

The instant disclosure comprises compounds of formula (I-B),

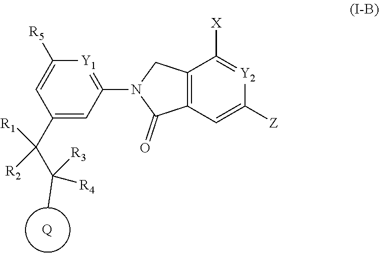

wherein:
Q is a 5-membered heteroaryl, optionally substituted by one or more alkyl, cycloalkyl, aryl, or haloalkyl groups;
Y₁ and Y₂ are independently CH, CF, or N;
R₁, R₂, R₃ and R₄ are each independently selected from: H, halo, alkyl, cycloalkyl,
CN, OH, alkoxy, and haloalkyl;
wherein at least one of R₁, R₂, R₃ and R₄ is halogen;
R₅ is selected from: H, halo, CN, or L₁ₐ-R₁₀, wherein L₁ₐ is —C(L_{1b}R₁₁)(R₁₂)—, —N(L_{1b}R₁₁)—, —C(═O)N(L_{1b}R₁₁)—, O, S, carbonyl, or a bond, wherein:
   L_{1b} is alkylene or a bond; and
   R₁₀, R₁₁ and R₁₂ are independently H, alkyl, alkoxyalkyl, aminoalkyl, cycloalkyl, bridged bicyclyl, fused bicyclyl, spirocyclyl, alkenyl, cycloalkenyl, alkynyl, amidoalkyl, aryl, heteroaryl, or heterocyclyl, and R₁₀, R₁₁ and R₁₂ are each optionally substituted by one or more groups selected from: halo, CN, amino, alkylamino, alkyl carbonyl, OH, oxo, alkoxy, alkyl, cycloalkyl, haloalkyl, alkoxyalkyl, sulfonamidyl, alkyl sulfonamidyl, heterocyclyl, aryl, and heteroaryl;
X is halo, haloalkyl, or cycloalkyl; and
Z is —L₂NR₇R₈, or —C(H)(NR₇R₈)R₆ₐ;
wherein:
L₂ is —C(H)R₆ₐ—, —C(═O)—, or a bond;
R₆ₐ=H, alkyl, cycloalkyl, or haloalkyl; and
R₇ and R₈ are independently selected from H, alkyl, cycloalkyl, spirocyclyl, bridged bicyclyl, hydroxyalkyl, aminoalkyl, heterocyclyl, and haloalkyl,
wherein, if any of R₇ or R₈ is alkyl, cycloalkyl, or heterocyclyl, said alkyl, cycloalkyl or heterocyclyl group is optionally substituted with one or more groups selected from: sulfonyl, halo, hydroxyl, alkoxy, alkyl, cycloalkyl, alkoxyalkyl, alkenyl, heterocyclyl, hydroxyalkyl, cyano, carboxylalkyl, and haloalkyl;
or
R₇ and R₈ together with the nitrogen atom to which they are both bonded form a:
   3-8 membered saturated monocyclic ring,
   wherein the saturated monocyclic ring is optionally substituted with one or more groups selected from: sulfonyl, halo, hydroxyl, alkoxy, alkyl, cycloalkyl, alkoxyalkyl, alkenyl, hydroxyalkyl, oxo, carboxyalkyl, or haloalkyl; with the provisos that:
      when Y₁ and Y₂ are both CH, R₁=F, R₂=methyl, R₃=R₄=F, R₅=H, L₂ is —CH₂—, X=CF₃, and Q is 2-methyl triazol-1-yl, the substituted saturated monocyclic ring is not 3-fluoro-azetidyn-1-yl, 3-cyano-azetidyn-1-yl, 3-methoxy-azetidyn-1-yl, 3-difluoromethyl-azetidyn-1-yl, 3-cyano-pyrrolidin-1-yl, 3-fluoro-pyrrolidin-1-yl, 3,4-difluoro-pyrrolidin-1-yl, 3,3-difluoro-pyrrolidin-1-yl or 3-methylsulfonyl-pyrrolidin-1-yl;
      when Y₁ and Y₂ are both CH, R₁=F, R₂=methyl, R₃=R₄=F, R₅=H, L₂ is —CH(CH₃)—, X=CF₃, and Q is 2-methyl triazol-1-yl, the substituted saturated monocyclic ring is not 3-fluoro-pyrrolidin-1-yl; and
      when Y₁ and Y₂ are both CH, R₁=F, R₂=methyl, R₃ is F, R₄ is F, R₅=H, L₂ is a bond, X=CF₃, and Q is 2-methyl triazol-1-yl, the substituted saturated monocyclic ring is not one of: piperazin-1-yl, 4-methyl-piperazin-1-yl, or 2,4-dimethyl-piperazin-1-yl;
   and
      when Y₁ and Y₂ are both CH, R₁=F, R₂=methyl, R₃ is F, R₄ is F, R₅=H, L₂ is C(═O), X=CF₃, and Q is 2-methyl triazol-1-yl, the substituted saturated monocyclic ring is not one of: 3-hydroxy-pyrrolidin-1-yl or 3-difluoromethyl-azetidin-1-yl;
or
R₇ and R₈ together with the nitrogen atom to which they are both bonded form a:
   3-8 membered saturated spirocyclic ring,
   wherein the saturated spirocyclic ring is optionally substituted with one or more groups selected from: sulfonyl, halo, hydroxyl, cyano, alkyl, alkenyl, cycloalkyl, alkoxy, alkoxyalkyl, hydroxyalkyl, oxo, carboxyalkyl, or haloalkyl;
or
R₇ and R₈ together with the nitrogen atom to which they are both bonded form a:
   3-8 membered saturated fused bicyclic ring,
   wherein the saturated fused bicyclic ring is optionally substituted with one or more groups selected from: sulfonyl, halo, hydroxyl, cyano, alkoxy, alkyl, cycloalkyl, alkoxyalkyl, alkenyl, hydroxyalkyl, oxo, carboxyalkyl, and haloalkyl;
or
R₇ and R₈ together with the nitrogen atom to which they are both bonded form a:
   3-8 membered saturated bridged bicyclic ring,
   wherein the saturated bridged bicyclic ring is optionally substituted with one or more groups selected from: sulfonyl, halo, hydroxyl, cyano, alkoxy, alkyl, cycloalkyl, alkoxyalkyl, alkenyl, hydroxyalkyl, oxo, carboxyalkyl, and haloalkyl; with the proviso that:
      when Y₁ and Y₂ are both CH, R₁ is F, R₂ is methyl, R₃ is F, R₄ is H, R₅=H, L₂ is a bond, X=CF₃, and Q is 2-methyl triazol-1-yl, the substituted saturated bridged bicyclic ring is not one of: 5-methyl-2,5-diazabicyclo[2.2.1]hept-2-yl, 2-methyl-2,5-diazabicyclo[2.2.2]oct-2-yl, 3-methyl-3,8-diazabicyclo[3.2.1]oct-8-yl, or 8-methyl-3,8-diazabicyclo[3.2.1]oct-3-yl;
or Z is

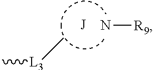

wherein:
   L₃ is —C(H)R_{6b}—, —N(R_{6b})—, O, —OC(H)(R_{6b})—, S, or a bond;
   R_{6b}=H, alkyl, cycloalkyl, or haloalkyl;
   J is a saturated 3-10 membered amine containing ring selected from a monocyclic ring, fused bicyclic ring, bridged bicyclic ring, and a spirocyclic ring, or a 5 or 6 member heteroaromatic ring, or a 3-10 member fused heteroaromatic ring system, and wherein:
   J is bonded to L₃ through a carbon atom; and
   J is optionally substituted by one or more sulfonyl, halo, hydroxyl, cyano, alkoxy, alkyl, cycloalkyl, alkoxyalkyl, alkenyl, hydroxyalkyl, oxo, carboxyalkyl, or haloalkyl groups; and
   R₉=H, alkyl, cycloalkyl, aminoalkyl, haloalkyl, or carboxyalkyl;
   with the provisos that,
      when L₃ is —N(Me)—, Y₁ and Y₂ are both CH, R₁ is F, R₂ is methyl, R₃ is F, R₄ is F, R₅=H, X=CF₃, R₉ is H, and Q is 2-methyl triazol-1-yl, J is not 4-fluoro-pyrrolidine-3-yl; and
      when L₃ is a bond, Y₁ and Y₂ are both CH, R₁ is F, R₂ is methyl, R₃ is F, R₄ is F, R₅=H, X=CF₃, and Q is 2-methyl triazol-1-yl, J is not 3-fluoro-pyridin-5-yl;
else Z is H;
and
with the provisos that, when Y₁ and Y₂ are both CH, R₂ is methyl, X=CF₃, and Q is 2-methyl triazol-1-yl:
   if R₃=R₄=F, and R₁=H, Z is not H;
   if R₃=F, R₄=H, and R₁ is F or H, Z is not H; and
   if R₃ and R₄ are both H, and R₁=F, Z is not H.
or an enantiomer, diastereomer or a pharmaceutically acceptable salt thereof.

In some embodiments, the compound has formula (I-B), wherein R₁ is F, R₂ is methyl, R₃ is F, and R₄ is H.

In some embodiments, the compound has formula (I-B), wherein R₁ is H or F, R₂ is F, and R₃=R₄=H.

In some embodiments, the compound has formula (I-B), wherein R₅ is H.

In some embodiments, the compound has formula (I-B), wherein Y₁=Y₂=CH.

In some embodiments, the compound has formula (I-B), wherein X=CF₃.

In some embodiments, the compound has formula (I-B), wherein Q is 2-methyl triazol-1-yl or imidazolyl.

In some embodiments, the compound has formula (I-B), wherein Z is —L₂NR₇R₈, and L₂ is CH₂ or C(H)Me.

In some embodiments, the compound has formula (I-B), wherein Z is —C(H)R₆NR₇R₈ and R₇ and R₈ together with the nitrogen atom to which they are both bonded form a 3-8 membered saturated monocyclic ring that is optionally substituted with one or more groups selected from: methyl, fluoromethyl, hydroxyethyl, chloromethyl, hydroxyl, propyl, isopropyl, methoxy, methoxymethyl, difluoromethyl, methoxyethyl, vinyl, methylsulfonyl, 2-fluoroethyl, acetyl, and 1,1,1-trifluoroethyl.

In some embodiments, the compound has formula (I-B), wherein Z is —L₂NR₇R₈, and L₂ is CH₂ or C(H)Me, and R₇ and R₈ and the nitrogen to which they are both bonded form a piperazin-1-yl ring substituted with one or more groups selected from: alkyl, sulfonyl, acetyl, haloalkyl, cycloalkyl, and oxetanyl.

In some embodiments, the compound has formula (I-B), wherein Z is —CH₂NHR₇, wherein R₇ is cycloalkyl substituted by an alkyl group.

In some embodiments, the compound has formula (I-B), wherein R₇ is 1-methyl-cyclobut-1-yl.

In some embodiments, the compound has formula (I-B), wherein R₅ is L₁ₐ-R₁₀.

The instant disclosure comprises compounds of formula (I-C),

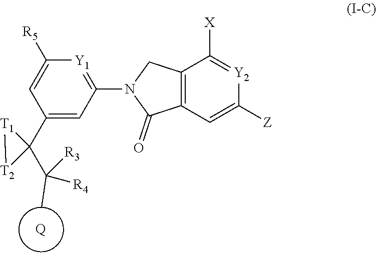

wherein:
Q is a 5-membered heteroaryl, optionally substituted by one or more alkyl, aryl, cycloalkyl, or haloalkyl groups;
Y₁ and Y₂ are independently CH, CF, or N;
T₁ and T₂ are each independently a group selected from: [—C(R₁)(R₂)—]ₙ, —O—, —C(R₁)(R₂)—O—, >C═O, —C(R₁)(R₂)—C(═O)—, —C(R₁)(R₂)—S(═O)₂—, and >S(═O)₂, with the proviso that T₁ and T₂ together are not —CH₂—O—, —O—O—, or —O—C(═O)—O—;
wherein n=0, 1 or 2, and each of R₁ and R₂ is independently selected from: H, halo, alkyl, alkenyl, cyano, hydroxyl, alkoxy, cycloalkyl, hydroxyalkyl, and haloalkyl, with the proviso that, when T₁ and T₂ together are —CH₂C(R₁)(R₂)CH₂—, neither of R₁ and R₂ is cyano;
or R₁ and R₂ together with the carbon atom to which they are both bonded form a cycloalkyl or heterocyclyl ring;
R₃, and R₄ are independently selected from: H, and halo, alkyl, CN, OH, alkoxy, and haloalkyl;
R₅ is selected from: H, halo, CN, or L₁ₐ-R₁₀, wherein L₁ₐ is —C(L_{1b}R₁₁)(R₁₂)—, —N(L_{1b}R₁₁)—, —C(═O)N(R₁₁)—, O, S, carbonyl, or a bond, wherein:
   L_{1b} is alkylene or a bond; and
   R₁₀, R₁₁ and R₁₂ are independently H, alkyl, alkoxyalkyl, aminoalkyl, cycloalkyl, bridged bicyclyl, fused bicyclyl, spirocyclyl, alkenyl, cycloalkenyl, alkynyl, amidoalkyl, aryl, heteroaryl, or heterocyclyl, and R₁₀, R₁₁ and R₁₂ are each optionally substituted by one or more groups selected from: halo, CN, amino, alkylamino, alkyl carbonyl, OH, oxo, alkoxy, alkyl, cycloalkyl, haloalkyl, alkoxyalkyl, sulfonamidyl, alkyl sulfonamidyl, heterocyclyl, aryl, and heteroaryl;
X is halo, haloalkyl, or cycloalkyl; and
Z is —L₂NR₇R₈ or —C(H)(NR₇R₈)R₆ₐ;
wherein:
L₂ is —C(H)R₆ₐ—, —C(═O)—, or a bond;
R₆ₐ=H, alkyl, cycloalkyl, or haloalkyl; and
R₇ and R₈ are independently selected from H, alkyl, spirocyclyl, cycloalkyl, bridged bicyclyl, hydroxyalkyl, heterocyclyl, and haloalkyl,
wherein, if any of R₇ or R₈ is alkyl, cycloalkyl or heterocyclyl, said alkyl, cycloalkyl or heterocyclyl group is optionally substituted with one or more groups selected from: sulfonyl, halo, hydroxyl, alkoxy, alkyl, cycloalkyl, alkoxyalkyl, alkenyl, heterocyclyl, hydroxyalkyl, cyano, carboxyalkyl, and haloalkyl;
or
R₇ and R₈ together with the nitrogen atom to which they are both bonded form a:
   3-8 membered saturated monocyclic ring,
   wherein the saturated monocyclic ring is optionally substituted with one or more groups selected from: sulfonyl, halo, hydroxyl, alkoxy, alkyl, cycloalkyl, alkoxyalkyl, alkenyl, hydroxyalkyl, oxo, carboxyalkyl, and haloalkyl; with the proviso that:
      when Y₁ and Y₂ are both CH, R₃=R₄=F, T₁ and T₂ are both CH₂, R₅=H, R₆ is methyl, X=CF₃, and Q is 2-methyl triazol-1-yl, the substituted saturated monocyclic ring is not 3-fluoro-pyrrolidin-1-yl;
or
R₇ and R₈ together with the nitrogen atom to which they are both bonded form a:
   3-8 membered saturated spirocyclic ring,
   wherein the saturated spirocyclic ring is optionally substituted with one or more groups selected from: sulfonyl, halo, hydroxyl, cyano, alkyl, alkenyl, cycloalkyl, alkoxy, alkoxyalkyl, hydroxyalkyl, oxo, carboxyalkyl, and haloalkyl;
   with the proviso that:
   when Y₁ and Y₂ are both CH, R₃ and R₄ are both H, T₁ is CH₂ or —(CH₂)—O—, T₂ is (CH₂)₂ R₅=H, R₆ is H or methyl, X=CF₃, and Q is 2-methyl triazol-1-yl, the substituted saturated spirocyclic ring is not 5-azaspiro[2.4]hept-5-yl;
or
R₇ and R₈ together with the nitrogen atom to which they are both bonded form a:
   3-8 membered saturated fused bicyclic ring,
   wherein the saturated fused bicyclic ring is optionally substituted with one or more groups selected from: sulfonyl, halo, hydroxyl, cyano, alkoxy, alkyl, cycloalkyl, alkoxyalkyl, alkenyl, hydroxyalkyl, oxo, carboxyalkyl, and haloalkyl;
or
R₇ and R₈ together with the nitrogen atom to which they are both bonded form a: 3-8 membered saturated bridged bicyclic ring, wherein the saturated bridged bicyclic ring is optionally substituted with one or more groups selected from: sulfonyl, halo, hydroxyl, cyano, alkoxy, alkyl, cycloalkyl, alkoxyalkyl, alkenyl, hydroxyalkyl, oxo, carboxyalkyl, and haloalkyl;
or Z is

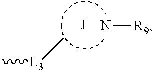

wherein:
   L₃ is —C(H)R_{6b}—, —N(R_{6b})—, O, —OC(H)(R_{6b})—, S, or a bond;
   R_{6b}=H, alkyl, cycloalkyl, or haloalkyl;
   J is a saturated 3-10 membered saturated amine containing ring selected from a monocyclic ring, spirocyclic ring, bridged bicyclic ring, or fused bicyclic ring, or a 5 or 6 member heteroaromatic ring, or a 3-10 member fused heteroaromatic ring system, and wherein:
      J is bonded to L₃ through a carbon atom; and
      J is optionally substituted by one or more sulfonyl, halo, hydroxyl, cyano, alkoxy, alkyl, cycloalkyl, alkoxyalkyl, alkenyl, hydroxyalkyl, oxo, carboxyalkyl, or haloalkyl groups; and
   R₉=H, alkyl, aminoalkyl, haloalkyl, cycloalkyl, or carboxyalkyl;
else
Z is H,
with the provisos that:
   when Y₁ and Y₂ are both CH, T=CF₂ or CH(CH₂OH) or a bond, R₃=R₄=H, R₅=H, R₆ is H, X=CF₃, and Q is 2-methyl triazol-1-yl, Z is not H;
or an enantiomer, diastereomer or a pharmaceutically acceptable salt thereof.

In some embodiments, the compound has formula (I-C), with the further proviso that T₁ and T₂ together are not —CH₂—O—CH₂—.

In some embodiments, the compound has formula (I-C), wherein R₅ is H.

In some embodiments, the compound has formula (I-C), wherein Y₁=Y₂=CH.

In some embodiments, the compound has formula (I-C), wherein X=CF₃.

In some embodiments, the compound has formula (I-C), wherein Q is 2-methyl triazol-1-yl or imidazolyl.

In some embodiments, the compound has formula (I-C), wherein Z is —L₂NR₇R₈, and L₂ is CH₂ or C(H)Me.

In some embodiments, the compound has formula (I-C), wherein Z is —C(H)R₆NR₇R₈ and R₇ and R₈ together with the nitrogen atom to which they are both bonded form a 3-8 membered saturated monocyclic ring that is optionally substituted with one or more groups selected from: methyl, fluoromethyl, hydroxyethyl, chloromethyl, hydroxyl, propyl, isopropyl, methoxy, methoxymethyl, difluoromethyl, methoxyethyl, vinyl, methylsulfonyl, 2-fluoroethyl, acetyl, and 1,1,1-trifluoroethyl.

In some embodiments, the compound has formula (I-C), wherein Z is —L₂NR₇R₈, and L₂ is CH₂ or C(H)Me, and R₇ and R₈ and the nitrogen to which they are both bonded form a piperazin-1-yl ring substituted with one or more groups selected from: alkyl, sulfonyl, acetyl, haloalkyl, cycloalkyl, and oxetanyl.

In some embodiments, the compound has formula (I-C), wherein Z is —CH₂NHR₇, wherein R₇ is cycloalkyl substituted by an alkyl group.

In some embodiments, the compound has formula (I-C), wherein R₇ is 1-methyl-cyclobut-1-yl.

In some embodiments, the compound has formula (I-C), wherein R₅ is L₁ₐ-R₁₀.

A compound of formula (I-D),

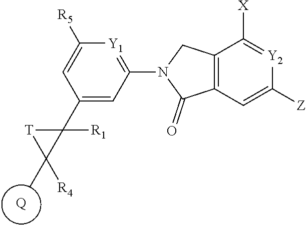

wherein:
Q is a 5-membered heteroaryl, optionally substituted by one or more alkyl, aryl, cycloalkyl, or haloalkyl groups;
Y₁ and Y₂ are independently CH, CF, or N;
T is a group selected from: [—C(R₂)(R₃)—]ₙ, —O—, —C(R₂)(R₃)—O—, >C═O, and >S(═O)₂;
wherein n=0, 1 or 2, and each of R₂ and R₃ is independently selected from: H, halo, alkyl, alkenyl, cyano, hydroxyl, alkoxy, cycloalkyl, hydroxyalkyl, and haloalkyl;
R₁ and R₄ are independently selected from: H, and halo, alkyl, CN, OH, alkoxy, and haloalkyl;
R₅ is selected from: H, halo, CN, or L₁ₐ-R₁₀, wherein L₁ₐ is —C(L_{1b}R₁₁)(R₁₂)—, —N(L_{1b}R₁₁)—, —C(═O)N(L_{1b}R₁₁)—, O, S, carbonyl, or a bond, wherein:
   L_{1b} is alkylene or a bond; and
   R₁₀, R₁₁ and R₁₂ are independently H, alkyl, alkoxyalkyl, aminoalkyl, cycloalkyl, bridged bicyclyl, fused bicyclyl, spirocyclyl, alkenyl, cycloalkenyl, alkynyl, amidoalkyl, aryl, heteroaryl, or heterocyclyl, and R₁₀, R₁₁ and R₁₂ are each optionally substituted by one or more groups selected from: halo, CN, amino, alkylamino, alkyl carbonyl, OH, oxo, alkoxy, alkyl, cycloalkyl, haloalkyl, alkoxyalkyl, sulfonamidyl, alkyl sulfonamidyl, heterocyclyl, aryl, and heteroaryl;
X is halo, haloalkyl, or cycloalkyl; and
Z is —L₂NR₇R₈, or —C(H)(NR₇R₈)R₆ₐ;
wherein:
L₂ is —C(H)R₆ₐ—, —C(═O)—, or a bond;
R₆ₐ=H, alkyl, cycloalkyl, or haloalkyl; and
R₇ and R₈ are independently selected from H, alkyl, cycloalkyl, spirocyclyl, bridged bicyclyl, hydroxyalkyl, heterocyclyl, and haloalkyl,
wherein, if any of R₇ or R₈ is alkyl, cycloalkyl or heterocyclyl, said alkyl, cycloalkyl or heterocyclyl group is optionally substituted with one or more groups selected from: sulfonyl, halo, hydroxyl, alkoxy, alkyl, cycloalkyl, alkoxyalkyl, alkenyl, heterocyclyl, hydroxyalkyl, cyano, carboxyalkyl, and haloalkyl;
or
R₇ and R₈ together with the nitrogen atom to which they are both bonded form a:
   3-10 membered cyclic group selected from a saturated monocyclic ring, spirocyclic ring, bridged bicyclic ring, or a fused bicyclic ring, wherein the 3-10 membered cyclic group is optionally substituted with one or more groups independently selected from: sulfonyl, halo, hydroxyl, alkoxy, alkyl, cycloalkyl, alkoxyalkyl, alkenyl, hydroxyalkyl, oxo, carboxyalkyl, and haloalkyl;
with the proviso that:
   when Y₁ and Y₂ are both CH, X=CF₃, R₅ is H, R₆ is H, and Q is 2-methyl triazol-1-yl:
   if T is CH₂, the 3-10 membered cyclic group is not 3-fluoro-pyrrolidin-1-yl;
or Z is

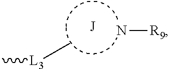

wherein:
   L₃ is —C(H)R₆—, —N(R₆)—, O, —OC(H)(R_{6b})—, S, or a bond;
   R_{6b}=H, alkyl, cycloalkyl, or haloalkyl;
   J is a saturated 3-10 membered amine containing ring selected from a monocyclic ring, spirocyclic ring, bridged bicyclic, ring, and a fused bicyclic ring, or a 5 or 6 member heteroaromatic ring, or a 3-10 member fused heteroaromatic ring system, and wherein:
      J is bonded to L₃ through a carbon atom; and
      J is optionally substituted by one or more sulfonyl, halo, hydroxyl, cyano, alkoxy, alkyl, cycloalkyl, alkoxyalkyl, alkenyl, hydroxyalkyl, oxo, carboxyalkyl, or haloalkyl groups; and
   R₉=H, alkyl, cycloalkyl, aminoalkyl, haloalkyl, or carboxyalkyl;
else
Z is H,
with the proviso that:
   when Y₁ and Y₂ are both CH, X=CF₃, R₅ is H, Q is 2-methyl triazol-1-yl, and T is CH(R₃), wherein R₂ is H or methyl, or T is [CH₂]₂, Z is not H;
or an enantiomer, diastereomer or a pharmaceutically acceptable salt thereof.

In some embodiments, the compound has formula (I-D), wherein T is —CH₂— or —(CH₂)₂—.

In some embodiments, the compound has formula (I-D), wherein Q is 2-methyl triazol-1-yl or imidazolyl.

In some embodiments, the compound has formula (I-D), wherein R₅ is H.

In some embodiments, the compound has formula (I-D), wherein Y₁=Y₂=CH.

In some embodiments, the compound has formula (I-D), wherein X=CF₃.

In some embodiments, the compound has formula (I-D), wherein Q is 2-methyl triazol-1-yl or imidazolyl.

In some embodiments, the compound has formula (I-D), wherein Z is —L₂NR₇R₈, and L₂ is CH₂ or C(H)Me.

In some embodiments, the compound has formula (I-D), wherein Z is —C(H)R₆NR₇R₈ and R₇ and R₈ together with the nitrogen atom to which they are both bonded form a 3-8 membered saturated monocyclic ring that is optionally substituted with one or more groups selected from: methyl, fluoromethyl, hydroxyethyl, chloromethyl, hydroxyl, propyl, isopropyl, methoxy, methoxymethyl, difluoromethyl, methoxyethyl, vinyl, methylsulfonyl, 2-fluoroethyl, acetyl, and 1,1,1-trifluoroethyl.

In some embodiments, the compound has formula (I-D), wherein Z is —L₂NR₇R₈, and L₂ is CH₂ or C(H)Me, and R₇ and R₈ and the nitrogen to which they are both bonded form a piperazin-1-yl ring substituted with one or more groups selected from: alkyl, sulfonyl, acetyl, haloalkyl, cycloalkyl, and oxetanyl.

In some embodiments, the compound has formula (I-D), wherein Z is —CH₂NHR₇, wherein R₇ is cycloalkyl substituted by an alkyl group.

In some embodiments, the compound has formula (I-D), wherein R₇ is 1-methyl-cyclobut-1-yl.

In some embodiments, the compound has formula (I-D), wherein R₅ is L₁ₐ-R₁₀.

A compound of formula (I-E),

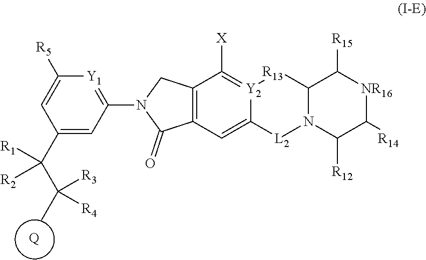

wherein:
Q is a 5-membered heteroaryl group, optionally substituted by one or more alkyl, aryl, cycloalkyl, or haloalkyl groups;
Y₁ and Y₂ are independently CH, CF, or N;
R₁, R₂, R₃, R₄ are independently selected from: H, halogen, alkyl, cycloalkyl, CN, OH, alkoxy, and haloalkyl; or
R₁ and R₂, together with the carbon atom to which they are both bonded form a 3-5 membered cycloalkyl or heterocyclyl, optionally substituted with one or more groups independently selected from: halogen, CN, OH, sulfonyl, alkoxy, alkyl, cycloalkyl, hydroxyalkyl, or haloalkyl; or
R₂ and R₃ together with the two carbon atoms to which they are respectively bonded form a 3-6 membered cycloalkyl, cycloalkenyl, heterocyclyl, heteroaryl ring, optionally substituted with one or more groups independently selected from: halogen, OH, alkoxy, cyano, sulfonyl, haloalkyl, hydroxyalkyl, alkyl, cycloalkyl, or alkoxyalkyl; R₅ is selected from: H, halo, CN, or L₁ₐ-R₆, wherein L₁ₐ is —C(L_{1b} R₇)(R₈)—, —N(L_{1b}R₇)—, —C(═O)N(L_{1b}R₇)—, O, S, carbonyl, or a bond, wherein:
   L_{1b} is alkylene or a bond; and
   R₆, R₇ and R₈ are independently H, alkyl, alkoxyalkyl, aminoalkyl, cycloalkyl, bridged bicyclyl, fused bicyclyl, spirocyclyl, alkenyl, cycloalkenyl, alkynyl, aryl, amidoalkyl, heteroaryl, or heterocyclyl, and R₆, R₇ and R₈ are optionally substituted by one or more groups selected from: halo, CN, amino, alkylamino, alkyl carbonyl, OH, oxo, alkoxy, alkyl, cycloalkyl, haloalkyl, alkoxyalkyl, sulfonamidyl, alkyl sulfonamidyl, heterocyclyl, aryl, and heteroaryl;
X is halo, haloalkyl, or cycloalkyl; and
L₂ is —C(H)R₉—, —C(═O)—, or a bond, wherein R₉=H, alkyl, cycloalkyl, or haloalkyl; R₁₂, R₁₃, R₁₄, R₁₅, and R₁₆ are each independently selected from: H, sulfonyl, halo, hydroxyl, alkoxy, alkyl, cycloalkyl, heterocyclyl, alkoxyalkyl, alkenyl, hydroxyalkyl, oxo, carboxyalkyl, and haloalkyl, wherein, if either of R₁₂, R₁₃, R₁₄, R₁₅, or R₁₆ is alkyl, alkenyl, or haloalkyl, then said alkyl, alkenyl, or haloalkyl is optionally substituted by one or more cycloalkyl or heterocyclyl groups; or
any pair of R₁₂, R₁₃, R₁₄, R₁₅, and R₁₆ together with the piperazine ring carbon atoms to which they are bonded form a cycloalkyl, or heterocyclyl, ring, wherein the ring is optionally substituted by one or more halo, hydroxyl, alkoxy, alkyl, cycloalkyl or heterocyclyl groups;
with the proviso that, when Y₁ and Y₂ are both CH, R₁=F, R₂=methyl, R₃=R₄=F, R₅=H, R₁₃=R₁₄=R₁₅=H, X=CF₃, Q is 2-methyl triazol-1-yl, and L₁ is a bond, then R₁₂ is not methyl, and R₁₆ is not H or methyl,
or an enantiomer, diastereomer or a pharmaceutically acceptable salt thereof.

In some embodiments, the compound has formula (I-E), wherein at least one of R₁, R₂, R₃ and R₄ is halogen.

In some embodiments, the compound has formula (I-E), wherein Q is 2-methyl triazol-1-yl or imidazolyl.

In some embodiments, the compound has formula (I-E), wherein X is CF₃.

In some embodiments, the compound has formula (I-E), wherein Y₁ and Y₂ are both CH.

In some embodiments, the compound has formula (I-E), wherein R₁ and R₂, together with the carbon atom to which they are both bonded form an oxetane ring.

In some embodiments, the compound has formula (I-E), wherein R₁ and R₂, together with the carbon atom to which they are both bonded form a ring selected from cyclopropyl, cyclobutyl or oxetane.

In some embodiments, the compound has formula (I-E), wherein R₂ and R₃ together with the two carbon atoms to which they are respectively bonded form a cyclopropyl or cyclobutyl ring.

In some embodiments, the compound has formula (I-E), wherein R₅ is H.

In some embodiments, the compound has formula (I-E), wherein L₂ is CH₂.

In some embodiments, the compound has formula (I-E), wherein one of R₁₂, R₁₃, R₁₄, R₁₅, and R₁₆ is isopropyl and the others are H.

In some embodiments, the compound has formula (I-E), wherein Z is —CH₂NHR₇, wherein R₇ is cycloalkyl substituted by an alkyl group.

In some embodiments, the compound has formula (I-E), wherein R₇ is 1-methyl-cyclobut-1-yl.

In some embodiments, the compound has formula (I-E), wherein R₅ is L₁ₐ-R₁₀.

A compound of formula (I-F),

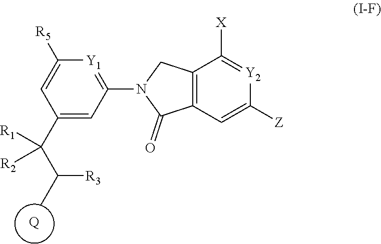

wherein:
Q is a 5-membered heteroaryl, optionally substituted by one or more alkyl, cycloalkyl, aryl, or haloalkyl groups;
Y₁ and Y₂ are independently CH, CF, or N;
R₁ is alkyl and R₂ is H, or R₁ and R₂ together are —CH₂OCH₂—;
R₃ is H or alkyl halogen;
R₅ is selected from: H, halo, CN, or L₁ₐ-R₁₀, wherein L₁ₐ is —C(L_{1b}R₁₁)(R₁₂)—, —N(L_{1b}R₁₁)—, —C(═O)N(L_{1b}R₁₁)—, O, S, carbonyl, or a bond, wherein:
   L_{1b} is alkylene or a bond; and
   R₁₀, R₁₁ and R₁₂ are independently H, alkyl, alkoxyalkyl, aminoalkyl, cycloalkyl, bridged bicyclyl, fused bicyclyl, spirocyclyl, alkenyl, cycloalkenyl, alkynyl, amidoalkyl, aryl, heteroaryl, or heterocyclyl, and R₁₀, R₁₁ and R₁₂ are each optionally substituted by one or more groups selected from: halo, CN, amino, alkylamino, alkyl carbonyl, OH, oxo, alkoxy, alkyl, cycloalkyl, haloalkyl, alkoxyalkyl, sulfonamidyl, alkyl sulfonamidyl, heterocyclyl, aryl, and heteroaryl;
X is halo, haloalkyl, or cycloalkyl; and
Z is —L₂NR₇R₈, or —C(H)(NR₇R₈)R₆ₐ;
wherein:
L₂ is —C(H)R₆ₐ—, —C(═O)—, or a bond;
R₆ₐ=H, alkyl, cycloalkyl, or haloalkyl; and
R₇ and R₈ are independently selected from H, alkyl, cycloalkyl, spirocyclyl, bridged bicyclyl, hydroxyalkyl, aminoalkyl, heterocyclyl, and haloalkyl,
wherein, if any of R₇ or R₈ is alkyl, cycloalkyl or heterocyclyl, said alkyl, cycloalkyl or heterocyclyl group is optionally substituted with one or more groups selected from: sulfonyl, halo, hydroxyl, alkoxy, alkyl, cycloalkyl, alkoxyalkyl, alkenyl, heterocyclyl, hydroxyalkyl, cyano, carboxyalkyl, and haloalkyl;
or
R₇ and R₈ together with the nitrogen atom to which they are both bonded form a 5-membered saturated monocyclic ring, optionally substituted with one or more groups selected from: sulfonyl, halo, alkoxy, alkyl, cycloalkyl, alkoxyalkyl, alkenyl, aminoalkyl, hydroxyalkyl, carboxyalkyl, and haloalkyl;
or
or Z is

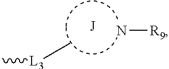

wherein:
   L₃ is —C(H)R_{6b}—, —N(R_{6b})—, O, —OC(H)(R_{6b})—, S, or a bond;
   R_{6b}=H, alkyl, cycloalkyl, or haloalkyl;
   J is a saturated 3-10 membered amine containing ring selected from a monocyclic ring, spirocyclic ring, bridged bicyclic ring, and a fused bicyclic ring, or a 5 or 6 member heteroaromatic ring, or a 3-10 member fused heteroaromatic ring system, and wherein:
      J is bonded to L₃ through a carbon atom; and
      J is optionally substituted by one or more sulfonyl, halo, hydroxyl, cyano, alkoxy, alkyl, cycloalkyl, alkoxyalkyl, alkenyl, hydroxyalkyl, oxo, carboxyalkyl, or haloalkyl groups; and
   R₉=H, alkyl, aminoalkyl, haloalkyl, or carboxyalkyl;
or an enantiomer, diastereomer or a pharmaceutically acceptable salt or solvate thereof.

In some embodiments, the compound has formula (I-F), wherein Z is

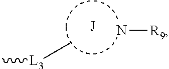

L₃ is a bond, R₉ is H, and J is a saturated monocyclic ring.

In some embodiments, the compound has formula (I-F), wherein J is a 5-membered saturated monocyclic ring.

In some embodiments, the compound has formula (I-F), wherein R₃ is H.

In some embodiments, the compound has formula (I-F), wherein R₅ is H.

In some embodiments, the compound has formula (I-F), wherein Y₁=Y₂=CH.

In some embodiments, the compound has formula (I-F), wherein X=CF₃.

In some embodiments, the compound has formula (I-F), wherein Q is 2-methyl triazol-1-yl or imidazolyl.

In some embodiments, the compound has formula (I-F), wherein R₇ is H and R₈ is alkyl.

In some embodiments, the compound has formula (I-F), wherein Z is —CH₂NHR₇, wherein R₇ is cycloalkyl substituted by an alkyl group.

In some embodiments, the compound has formula (I-F), wherein R₇ is 1-methyl-cyclobut-1-yl.

In some embodiments, the compound has formula (I-F), wherein R₅ is L₁ₐ-R₁₀.

A compound of formula (I-G),

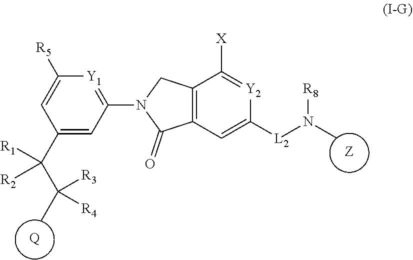

wherein:
Q is a 5-membered heteroaryl group, optionally substituted by one or more aryl, alkyl, cycloalkyl, or haloalkyl groups;
Y₁ and Y₂ are independently CH, CF, or N;
R₁, R₂, R₃, R₄ are independently selected from: H, halogen, alkyl, cycloalkyl, CN, OH, alkoxy, and haloalkyl; or
R₁ and R₂, together with the carbon atom to which they are both bonded form a 3-5 membered cycloalkyl or heterocyclyl ring, optionally substituted with one or more groups independently selected from: halogen, CN, OH, sulfonyl, alkoxy, alkyl, cycloalkyl, hydroxyalkyl, or haloalkyl;
R₅ is selected from: H, halo, CN, or L₁ₐ-R₆, wherein L₁ₐ is —C(L_{1b} R₇)(R₈)—, —N(L_{1b}R₇)—, —C(═O)N(L_{1b}R₇)—, O, S, carbonyl, or a bond, wherein:
   L_{1b} is alkylene or a bond; and
   R₆, R₇ and R₈ are independently H, alkyl, alkoxyalkyl, aminoalkyl, cycloalkyl, bridged bicyclyl, fused bicyclyl, spirocyclyl, alkenyl, cycloalkenyl, alkynyl, aryl, amidoalkyl, heteroaryl, or heterocyclyl, and R₆, R₇ and R₈ are optionally substituted by one or more groups selected from: halo, CN, amino, alkylamino, alkyl carbonyl, OH, oxo, alkoxy, alkyl, cycloalkyl, haloalkyl, alkoxyalkyl, sulfonamidyl, alkyl sulfonamidyl, heterocyclyl, aryl, and heteroaryl;
X is halo, haloalkyl, or cycloalkyl;
L₂ is —C(H)R₉—, —C(═O)—, or a bond, wherein R₉=H, alkyl, cycloalkyl, or haloalkyl; and
Z is a 3-10 membered ring system, optionally substituted by one or more substituents independently selected from: H, sulfonyl, halo, hydroxyl, alkoxy, alkyl, cycloalkyl, heterocyclyl, alkoxyalkyl, alkenyl, hydroxyalkyl, oxo, carboxylalkyl, and haloalkyl;
or an enantiomer, diastereomer or a pharmaceutically acceptable salt or solvate thereof.

In some embodiments, the compound has formula (I-G), wherein Z is a monocyclic ring substituted by an alkyl group.

In some embodiments, the compound has formula (I-G), wherein at least one of R₁, R₂, R₃ and R₄ is halogen.

In some embodiments, the compound has formula (I-G), wherein Q is 2-methyl triazol-1-yl or imidazolyl.

In some embodiments, the compound has formula (I-G), wherein X is CF₃.

In some embodiments, the compound has formula (I-G), wherein Y₁ and Y₂ are both CH.

In some embodiments, the compound has formula (I-G), wherein R₁ and R₂, together with the carbon atom to which they are both bonded form an oxetane ring.

In some embodiments, the compound has formula (I-G), wherein R₁ and R₂, together with the carbon atom to which they are both bonded form a ring selected from cyclopropyl, cyclobutyl or oxetane.

In some embodiments, the compound has formula (I-G), wherein Z is —CH₂NHR₇, wherein R₇ is cycloalkyl substituted by an alkyl group.

In some embodiments, the compound has formula (I-G), wherein R₇ is 1-methyl-cyclobut-1-yl.

In some embodiments, the compound has formula (I-G), wherein R₅ is L₁ₐ-R₁₀.

In some embodiments, the compound has formula (I-G), wherein R₅ is H.

In some embodiments, the compound has formula (I-A), (I-B), (I-C), (I-D), (I-E), (I-F), or (I-G), wherein Q is 4-methyl-2H-1,2,3-triazol-2-yl, 4-methyl-1H-1,2,3-triazol-1-yl, 5-methyl-1H-1,2,3-triazol-1-yl, 5-methyl-1H-pyrazol-1-yl, 3-methyl-1H-pyrazol-1-yl, 1-methyl-1H-imidazol-2-yl, or 4-methyl-4H-1,2,4-triazol-3-yl.

Any compound as explicitly identified herein is also to be considered within the scope of the present invention, including but not limited to the following, and pharmaceutically acceptable salts, hydrates, or solvates thereof. The lettering scheme (a), b), c) . . . etc.), per page in the following list is for ease of viewing on a page by page basis.

| | |
|---|---|
| a) | (R)-6-(2-oxa-6-azaspiro[3.3]heptan-6- |
| | ylmethyl)-2-(3-(3-(fluoro(4-methyl- |
| | 4H-1,2,4-triazol-3-yl)methyl)oxetan-3- |
| | yl)phenyl)-4-(trifluoromethyl)-isoindolin-1-one |
| b) | (S)-6-(2-oxa-6-azaspiro[3.3]heptan-6- |
| | ylmethyl)-2-(3-(3-(fluoro(4-methyl- |
| | 4H-1,2,4-triazol-3-yl)methyl)oxetan- |
| | 3-yl)phenyl)-4-(trifluoromethyl)- |
| | isoindolin-1-one |
| c) | (±)-2-(3-(3-((4-methyl-4H-1,2,4-triazol- |
| | 3-yl)methyl)oxetan-3-yl)phenyl)-6-(pyrrolidin- |
| | 2-yl)-4-(trifluoromethyl)isoindolin-1-one formate |
| a) | 6-(azetidin-3-yl)-2-(3-(3-((4-methyl- |
| | 4H-1,2,4-triazol-3-yl)methyl)oxetan- |
| | 3-yl)phenyl)-4-(trifluoromethyl)isoindolin-1-one |
| b) | (±)-6-(azetidin-2-yl)-2-(3-(3-((4-methyl-4H-1,2,4- |
| | triazol-3-yl)methyl)oxetan-3-yl)phenyl)-4- |
| | (trifluoromethyl)isoindolin-1-one |
| | formate |
| c) | (±)-2-(3-(1-fluoro-2-(4-methyl-4H- |
| | 1,2,4-triazol-3-yl)ethyl)phenyl)-4- |
| | (trifluoromethyl)isoindolin-1-one |
| d) | 2-(3-(1,1-difluoro-2-(4-methyl-4H- |
| | 1,2,4-triazol-3-yl)ethyl)phenyl)-4- |
| | (trifluoromethyl)isoindolin-1-one |
| e) | 2-(3-(3,3-difluoro-1-((4-methyl-4H-1,2,4-triazol-3- |
| | yl)methyl)cyclobutyl)phenyl)-6- |
| | ((3-hydroxy-3-methylazetidin-1- |
| | yl)methyl)-4-(trifluoromethyl)isoindolin-1-one |
| f) | (R)-6-(5-azaspiro[2.4]heptan-5- |
| | ylmethyl)-2-(3-(1-(fluoro(4-methyl-4H- |
| | 1,2,4-triazol-3-yl)methyl)cyclopropyl)phenyl)-4- |
| | (trifluoromethyl)isoindolin-1-one |
| g) | (S)-6-(5-azaspiro[2.4]heptan-5-ylmethyl)- |
| | 2-(3-(1-(fluoro(4-methyl-4H- |
| | 1,2,4-triazol-3-yl)methyl)cyclopropyl)phenyl)-4- |
| | (trifluoromethyl)isoindolin-1-one |
| h) | (S)-2-(3-(1-(fluoro(4-methyl-4H-1,2,4-triazol-3- |
| | yl)methyl)cyclopropyl)phenyl)-6- |
| | ((3-hydroxy-3-methylazetidin-1- |
| | yl)methyl)-4-(trifluoromethyl)isoindolin-1-one |
| i) | (R)-2-(3-(1-(fluoro(4-methyl-4H-1,2,4-triazol-3- |
| | yl)methyl)cyclopropyl)phenyl)-6- |
| | ((3-hydroxy-3-methylazetidin-1- |
| | yl)methyl)-4-(trifluoromethyl)isoindolin-1-one |
| j) | 2-(3-((1S,2R)-1,2-difluoro-1-(4-methyl- |
| | 4H-1,2,4-triazol-3-yl)propan-2- |
| | yl)phenyl)-6-((3-fluoro-3-methylazetidin-1- |
| | yl)methyl)-4-(trifluoromethyl)isoindolin-1-one |
| k) | 2-(3-((1R,2S)-1,2-difluoro-1-(4-methyl- |
| | 4H-1,2,4-triazol-3-yl)propan-2- |
| | yl)phenyl)-6-((3-fluoro-3-methylazetidin-1- |
| | yl)methyl)-4-(trifluoromethyl)isoindolin-1-one |
| a) | 2-(3-((1S,2S)-1,2-difluoro-1-(4-methyl- |
| | 4H-1,2,4-triazol-3-yl)propan-2- |
| | yl)phenyl)-6-((3-fluoro-3-methylazetidin-1- |
| | yl)methyl)-4-(trifluoromethyl)isoindolin-1-one |
| b) | 2-(3-((1R,2R)-1,2-difluoro-1-(4-methyl- |
| | 4H-1,2,4-triazol-3-yl)propan-2- |
| | yl)phenyl)-6-((3-fluoro-3-methylazetidin-1- |
| | yl)methyl)-4-(trifluoromethyl)isoindolin-1-one |
| c) | 2-(3-(3-((R)-fluoro(4-methyl-4H- |
| | 1,2,4-triazol-3-yl)methyl)oxetan-3- |
| | yl)phenyl)-6-(((R)-3-methoxypyrrolidin- |
| | 1-yl)methyl)-4-(trifluoromethyl)isoindolin-1-one |
| d) | 2-(3-(3-((S)-fluoro(4-methyl-4H- |
| | 1,2,4-triazol-3-yl)methyl)oxetan-3- |
| | yl)phenyl)-6-(((R)-3-methoxypyrrolidin- |
| | 1-yl)methyl)-4-(trifluoromethyl)isoindolin-1-one |
| e) | 2-(3-(1-(difluoro(4-methyl-4H-1,2,4-triazol-3- |
| | yl)methyl)cyclopropyl)phenyl)-4- |
| | (trifluoromethyl)isoindolin-1-one |
| f) | (±)-2-(3-(3-((4-methyl-4H-1,2,4-triazol- |
| | 3-yl)methyl)oxetan-3-yl)phenyl)-6-(pyrrolidin- |
| | 3-yl)-4-(trifluoromethyl)isoindolin-1-one formate |
| g) | (R)-2-(3-(3,3-difluoro-1-(fluoro(4-methyl-4H- |
| | 1,2,4-triazol-3-yl)methyl)cyclobutyl)phenyl)- |
| | 6-((3-hydroxy-3-methylazetidin-1- |
| | yl)methyl)-4-(trifluoromethyl)isoindolin-1-one |
| h) | (S)-2-(3-(3,3-difluoro-1-(fluoro(4-methyl-4H- |
| | 1,2,4-triazol-3-yl)methyl)cyclobutyl)phenyl)- |
| | 6-((3-hydroxy-3-methylazetidin-1- |
| | yl)methyl)-4-(trifluoromethyl)isoindolin-1-one |
| i) | 6-((S)-amino(cyclopropyl)methyl)-2-(3- |
| | (3-((R)-fluoro(4-methyl-4H-1,2,4- |
| | triazol-3-yl)methyl)oxetan-3-yl)phenyl)- |
| | 4-(trifluoromethyl)isoindolin-1- |
| | one |
| j) | 6-((R)-amino(cyclopropyl)methyl)-2- |
| | (3-(3-((R)-fluoro(4-methyl-4H-1,2,4- |
| | triazol-3-yl)methyl)oxetan-3-yl)phenyl)- |
| | 4-(trifluoromethyl)isoindolin-1- |
| | one |
| k) | (R)-6-(5-azaspiro[2.4]heptan-5-ylmethyl)- |
| | 2-(6-(isopropylamino)-4-(1-(4-methyl- |
| | 4H-1,2,4-triazol-3-yl)propan-2-yl)pyridin-2-yl)-4- |
| | (trifluoromethyl)isoindolin-1-one formate |
| a) | (R)-6-(5-azaspiro[2.4]heptan-5- |
| | ylmethyl)-2-(6-ethoxy-4-(1-(4-methyl- |
| | 4H-1,2,4-triazol-3-yl)propan-2-yl)pyridin-2-yl)-4- |
| | (trifluoromethyl)isoindolin-1-one formate |
| b) | ((R)-6-(azetidin-3-ylmethyl)-2-(3-(3- |
| | (fluoro(4-methyl-4H-1,2,4-triazol-3- |
| | yl)methyl)oxetan-3-yl)phenyl)-4- |
| | (trifluoromethyl)isoindolin-1-one |
| c) | (S)-6-(azetidin-3-ylmethyl)-2-(3-(3- |
| | (fluoro(4-methyl-4H-1,2,4-triazol-3- |
| | yl)methyl)oxetan-3-yl)phenyl)-4- |
| | (trifluoromethyl)isoindolin-1-one |
| d) | (R)-4-(difluoromethyl)-2-(3-(3- |
| | (fluoro(4-methyl-4H-1,2,4-triazol-3- |
| | yl)methyl)oxetan-3-yl)phenyl)isoindolin-1-one |
| e) | 6-(aminomethyl)-2-(3-(3-((4-methyl-4H- |
| | 1,2,4-triazol-3-yl)methyl)oxetan-3-yl)phenyl)- |
| | 4-(trifluoromethyl)isoindolin-1-one formate |
| f) | (R)-6-(aminomethyl)-2-(3-(3-(fluoro(4- |
| | methyl-4H-1,2,4-triazol-3- |
| | yl)methyl)oxetan-3-yl)phenyl)-4- |
| | (trifluoromethyl)isoindolin-1-one |
| g) | (S)-6-(aminomethyl)-2-(3-(3-(fluoro(4- |
| | methyl-4H-1,2,4-triazol-3- |
| | yl)methyl)oxetan-3-yl)phenyl)-4- |
| | (trifluoromethyl)isoindolin-1-one |
| h) | (R)-6-(azetidin-1-ylmethyl)-2-(3-(3- |
| | (fluoro(4-methyl-4H-1,2,4-triazol-3- |
| | yl)methyl)oxetan-3-yl)phenyl)-4- |
| | (trifluoromethyl)isoindolin-1-one |
| i) | (S)-6-(azetidin-1-ylmethyl)-2-(3-(3- |
| | (fluoro(4-methyl-4H-1,2,4-triazol-3- |
| | yl)methyl)oxetan-3-yl)phenyl)-4- |
| | (trifluoromethyl)isoindolin-1-one |
| j) | (R)-6-((3,3-dimethylazetidin-1-yl)methyl)- |
| | 2-(3-(3-(fluoro(4-methyl-4H- |
| | 1,2,4-triazol-3-yl)methyl)oxetan-3-yl)phenyl)-4- |
| | (trifluoromethyl)isoindolin-1-one |
| k) | (S)-6-((3,3-dimethylazetidin-1-yl)methyl)- |
| | 2-(3-(3-(fluoro(4-methyl-4H- |
| | 1,2,4-triazol-3-yl)methyl)oxetan-3-yl)phenyl)-4- |
| | (trifluoromethyl)isoindolin-1-one |
| l) | (R)-2-(3-(3-(fluoro(4-methyl-4H-1,2,4- |
| | triazol-3-yl)methyl)oxetan-3- |
| | yl)phenyl)-6-((3-(hydroxymethyl)-3- |
| | methylazetidin-1-yl)methyl)-4- |
| | (trifluoromethyl)isoindolin-1-one |
| m) | (S)-2-(3-(3-(fluoro(4-methyl-4H-1,2,4- |
| | triazol-3-yl)methyl)oxetan-3- |
| | yl)phenyl)-6-((3-(hydroxymethyl)-3- |
| | methylazetidin-1-yl)methyl)-4- |
| | (trifluoromethyl)isoindolin-1-one |
| a) | (R)-2-(3-(3-(fluoro(4-methyl-4H-1,2,4- |
| | triazol-3-yl)methyl)oxetan-3- |
| | yl)phenyl)-6-((3-(fluoromethyl)- |
| | 3-methylazetidin-1-yl)methyl)-4- |
| | (trifluoromethyl)isoindolin-1-one |
| b) | (S)-2-(3-(3-(fluoro(4-methyl-4H- |
| | 1,2,4-triazol-3-yl)methyl)oxetan-3- |
| | yl)phenyl)-6-((3-(fluoromethyl)-3- |
| | methylazetidin-1-yl)methyl)-4- |
| | (trifluoromethyl)isoindolin-1-one |
| c) | (R)-6-((5-azaspiro[2.3]hexan-5-yl)methyl)- |
| | 2-(3-(3-(fluoro(4-methyl-4H- |
| | 1,2,4-triazol-3-yl)methyl)oxetan-3-yl)phenyl)-4- |
| | (trifluoromethyl)isoindolin-1-one |
| d) | (S)-6-((5-azaspiro[2.3]hexan-5-yl)methyl)- |
| | 2-(3-(3-(fluoro(4-methyl-4H- |
| | 1,2,4-triazol-3-yl)methyl)oxetan-3-yl)phenyl)-4- |
| | (trifluoromethyl)isoindolin-1-one |
| e) | 2-(3-(3-((R)-fluoro(4-methyl-4H-1,2,4- |
| | triazol-3-yl)methyl)oxetan-3-yl)phenyl)- |
| | 6-(((R)-3-fluoropyrrolidin-1-yl)methyl)-4- |
| | (trifluoromethyl)isoindolin-1-one |
| f) | 2-(3-(3-((S)-fluoro(4-methyl-4H-1,2,4- |
| | triazol-3-yl)methyl)oxetan-3-yl)phenyl)- |
| | 6-(((R)-3-fluoropyrrolidin-1-yl)methyl)-4- |
| | (trifluoromethyl)isoindolin-1-one |
| g) | 2-(3-(3-((R)-fluoro(4-methyl-4H-1,2,4- |
| | triazol-3-yl)methyl)oxetan-3-yl)phenyl)- |
| | 6-(((S)-3-fluoropyrrolidin-1-yl)methyl)-4- |
| | (trifluoromethyl)isoindolin-1-one |
| h) | 2-(3-(3-((S)-fluoro(4-methyl-4H-1,2,4- |
| | triazol-3-yl)methyl)oxetan-3-yl)phenyl)- |
| | 6-(((S)-3-fluoropyrrolidin-1-yl)methyl)-4- |
| | (trifluoromethyl)isoindolin-1-one |
| i) | 6-((3-azabicyclo[3.1.0]hexan-3-yl)methyl)- |
| | 2-(3-(3-((R)-fluoro(4-methyl-4H-1,2,4- |
| | triazol-3-yl)methyl)oxetan-3-yl)phenyl)-4- |
| | (trifluoromethyl)isoindolin-1-one |
| j) | 6-((3-azabicyclo[3.1.0]hexan-3-yl)methyl)- |
| | 2-(3-(3-((S)-fluoro(4-methyl-4H-1,2,4- |
| | triazol-3-yl)methyl)oxetan-3-yl)phenyl)-4- |
| | (trifluoromethyl)isoindolin-1-one |
| a) | (R)-6-((2-azabicyclo[2.1.1]hexan-2-yl)methyl)- |
| | 2-(3-(3-(fluoro(4-methyl-4H-1,2,4- |
| | triazol-3-yl)methyl)oxetan-3-yl)phenyl)-4- |
| | (trifluoromethyl)isoindolin-1-one |
| b) | (S)-6-((2-azabicyclo[2.1.1]hexan-2- |
| | yl)methyl)-2-(3-(3-(fluoro(4-methyl-4H-1,2,4- |
| | triazol-3-yl)methyl)oxetan-3-yl)phenyl)-4- |
| | (trifluoromethyl)isoindolin-1-one |
| c) | (R)-2-(3-(3-(fluoro(4-methyl-4H-1,2,4- |
| | triazol-3-yl)methyl)oxetan-3- |
| | yl)phenyl)-6-(morpholinomethyl)-4- |
| | (trifluoromethyl)isoindolin-1-one |
| d) | (S)-2-(3-(3-(fluoro(4-methyl-4H-1,2,4- |
| | triazol-3-yl)methyl)oxetan-3- |
| | yl)phenyl)-6-(morpholinomethyl)-4- |
| | (trifluoromethyl)isoindolin-1-one |
| e) | 2-(3-(3-((R)-fluoro(4-methyl-4H- |
| | 1,2,4-triazol-3-yl)methyl)oxetan-3-yl)phenyl)- |
| | 6-(((S)-3-isopropylpiperazin-1-yl)methyl)-4- |
| | (trifluoromethyl)isoindolin-1-one |
| f) | 2-(3-(3-((S)-fluoro(4-methyl-4H-1,2,4- |
| | triazol-3-yl)methyl)oxetan-3-yl)phenyl)- |
| | 6-(((S)-3-isopropylpiperazin-1-yl)methyl)-4- |
| | (trifluoromethyl)isoindolin-1-one |
| g) | 2-(3-(3-((R)-fluoro(4-methyl-4H-1,2,4- |
| | triazol-3-yl)methyl)oxetan-3-yl)phenyl)- |
| | 6-(((R)-3-isopropylpiperazin-1-yl)methyl)-4- |
| | (trifluoromethyl)isoindolin-1-one |
| h) | 2-(3-(3-((S)-fluoro(4-methyl-4H-1,2,4- |
| | triazol-3-yl)methyl)oxetan-3-yl)phenyl)- |
| | 6-(((R)-3-isopropylpiperazin-1-yl)methyl)-4- |
| | (trifluoromethyl)isoindolin-1-one |
| i) | 2-(3-(3-((R)-fluoro(4-methyl-4H-1,2,4- |
| | triazol-3-yl)methyl)oxetan-3-yl)phenyl)- |
| | 6-(((R)-2-isopropylpiperazin-1-yl)methyl)-4- |
| | (trifluoromethyl)isoindolin-1-one |
| j) | 2-(3-(3-((S)-fluoro(4-methyl-4H-1,2,4- |
| | triazol-3-yl)methyl)oxetan-3-yl)phenyl)- |
| | 6-(((R)-2-isopropylpiperazin-1-yl)methyl)-4- |
| | (trifluoromethyl)isoindolin-1-one |
| k) | 2-(3-(3-((R)-fluoro(4-methyl-4H-1,2,4- |
| | triazol-3-yl)methyl)oxetan-3-yl)phenyl)- |
| | 6-(((S)-2-isopropylpiperazin-1-yl)methyl)-4- |
| | (trifluoromethyl)isoindolin-1-one |
| a) | 2-(3-(3-((S)-fluoro(4-methyl-4H-1,2,4- |
| | triazol-3-yl)methyl)oxetan-3-yl)phenyl)- |
| | 6-(((S)-2-isopropylpiperazin-1-yl)methyl)-4- |
| | (trifluoromethyl)isoindolin-1-one |
| b) | 2-(3-((1S,3R)-3-fluoro-1-((4-methyl-4H-1,2,4- |
| | triazol-3-yl)methyl)cyclobutyl)phenyl)-4- |
| | (trifluoromethyl)isoindolin-1-one |
| c) | 2-(3-((1R,3S)-3-fluoro-1-((4-methyl-4H-1,2,4- |
| | triazol-3-yl)methyl)cyclobutyl)phenyl)-4- |
| | (trifluoromethyl)isoindolin-1-one |
| d) | (R)-6-((3-(chloromethyl)pyrrolidin-1- |
| | yl)methyl)-2-(3-(3-((4-methyl-4H- |
| | 1,2,4-triazol-3-yl)methyl)oxetan-3-yl)phenyl)-4- |
| | (trifluoromethyl)isoindolin-1-one |
| e) | (S)-6-((3-(chloromethyl)pyrrolidin-1- |
| | yl)methyl)-2-(3-(3-((4-methyl-4H- |
| | 1,2,4-triazol-3-yl)methyl)oxetan-3-yl)phenyl)-4- |
| | (trifluoromethyl)isoindolin-1-one |
| f) | (R)-6-((3-(chloromethyl)-3-methylazetidin- |
| | 1-yl)methyl)-2-(3-(3-(fluoro(4- |
| | methyl-4H-1,2,4-triazol-3-yl)methyl)oxetan-3- |
| | yl)phenyl)-4-(trifluoromethyl)isoindolin-1-one |
| g) | (S)-6-((3-(chloromethyl)-3-methylazetidin- |
| | 1-yl)methyl)-2-(3-(3-(fluoro(4- |
| | methyl-4H-1,2,4-triazol-3-yl)methyl)oxetan-3- |
| | yl)phenyl)-4-(trifluoromethyl)isoindolin-1-one |
| h) | 2-(3-(3,3-difluoro-1-((4-methyl-4H-1,2,4-triazol-3- |
| | yl)methyl)cyclobutyl)phenyl)-6-((2,6- |
| | dihydropyrrolo[3,4-c]pyrazol-5(4H)- |
| | yl)methyl)-4-(trifluoromethyl)isoindolin-1-one |
| i) | 6-((5-azaspiro[2.4]heptan-5-yl)methyl)- |
| | 2-(3-(3,3-difluoro-1-((4-methyl- |
| | 4H-1,2,4-triazol-3-yl)methyl)cyclobutyl)phenyl)-4- |
| | (trifluoromethyl)isoindolin-1-one |
| j) | 2-(3-(3,3-difluoro-1-((4-methyl-4H-1,2,4-triazol-3- |
| | yl)methyl)cyclobutyl)phenyl)-6-((3-fluoro- |
| | 3-methylazetidin-1-yl)methyl)- |
| | 4-(trifluoromethyl)isoindolin-1-one |
| k) | 2-(3-(3-((R)-fluoro(4-methyl-4H-1,2,4- |
| | triazol-3-yl)methyl)oxetan-3- |
| | yl)phenyl)-6-((S)-1-(3-fluoro-3- |
| | methylazetidin-1-yl)ethyl)-4- |
| | (trifluoromethyl)isoindolin-1-one |
| a) | 2-(3-(3-((R)-fluoro(4-methyl-4H-1,2,4- |
| | triazol-3-yl)methyl)oxetan-3- |
| | yl)phenyl)-6-((R)-1-(3-fluoro-3- |
| | methylazetidin-1-yl)ethyl)-4- |
| | (trifluoromethyl)isoindolin-1-one |
| b) | 2-(3-(3-((S)-fluoro(4-methyl-4H-1,2,4- |
| | triazol-3-yl)methyl)oxetan-3- |
| | yl)phenyl)-6-((S)-1-(3-fluoro-3- |
| | methylazetidin-1-yl)ethyl)-4- |
| | (trifluoromethyl)isoindolin-1-one |
| c) | 2-(3-(3-((S)-fluoro(4-methyl-4H-1,2,4- |
| | triazol-3-yl)methyl)oxetan-3- |
| | yl)phenyl)-6-((R)-1-(3-fluoro- |
| | 3-methylazetidin-1-yl)ethyl)-4- |
| | (trifluoromethyl)isoindolin-1-one |
| d) | (R)-6-((1H-pyrazol-4-yl)oxy)-2-(3-(3- |
| | (fluoro(4-methyl-4H-1,2,4-triazol-3- |
| | yl)methyl)oxetan-3-yl)phenyl)-4- |
| | (trifluoromethyl)isoindolin-1-one |
| e) | (S)-6-((1H-pyrazol-4-yl)oxy)-2-(3-(3- |
| | (fluoro(4-methyl-4H-1,2,4-triazol-3- |
| | yl)methyl)oxetan-3-yl)phenyl)-4- |
| | (trifluoromethyl)isoindolin-1-one |
| f) | 2-(3-(3-((R)-fluoro(4-methyl-4H-1,2,4- |
| | triazol-3-yl)methyl)oxetan-3-yl)phenyl)- |
| | 6-(((S)-2-methyl-1,4-oxazepan-4-yl)methyl)-4- |
| | (trifluoromethyl)isoindolin-1-one |
| g) | 2-(3-(3-((R)-fluoro(4-methyl-4H-1,2,4- |
| | triazol-3-yl)methyl)oxetan-3-yl)phenyl)- |
| | 6-(((R)-2-methyl-1,4-oxazepan-4-yl)methyl)-4- |
| | (trifluoromethyl)isoindolin-1-one |
| h) | 2-(3-(3-((S)-fluoro(4-methyl-4H-1,2,4- |
| | triazol-3-yl)methyl)oxetan-3-yl)phenyl)- |
| | 6-((2-methyl-1,4-oxazepan-4-yl)methyl)-4- |
| | (trifluoromethyl)isoindolin-1-one |
| i) | (S)-2-(3-(1-(fluoro(4-methyl-4H-1,2,4-triazol-3- |
| | yl)methyl)cyclopropyl)phenyl)-6-((3- |
| | (hydroxymethyl)-3-methylazetidin-1- |
| | yl)methyl)-4-(trifluoromethyl)isoindolin-1-one |
| j) | (R)-2-(3-(1-(fluoro(4-methyl-4H-1,2,4-triazol-3- |
| | yl)methyl)cyclopropyl)phenyl)-6-((3- |
| | (hydroxymethyl)-3-methylazetidin-1- |
| | yl)methyl)-4-(trifluoromethyl)isoindolin-1-one |
| k) | 6-(5-azaspiro[2.4]heptan-5-ylmethyl)- |
| | 2-(3-((1S,2R)-1,2-difluoro-1-(4-methyl-4H- |
| | 1,2,4-triazol-3-yl)propan-2-yl)phenyl)-4- |
| | (trifluoromethyl)isoindolin-1-one |
| a) | 6-(5-azaspiro[2.4]heptan-5-ylmethyl)- |
| | 2-(3-((1S,2S)-1,2-difluoro-1-(4-methyl-4H- |
| | 1,2,4-triazol-3-yl)propan-2-yl)phenyl)-4- |
| | (trifluoromethyl)isoindolin-1-one |
| b) | 6-(5-azaspiro[2.4]heptan-5-ylmethyl)- |
| | 2-(3-((1R,2R)-1,2-difluoro-1-(4-methyl-4H- |
| | 1,2,4-triazol-3-yl)propan-2-yl)phenyl)-4- |
| | (trifluoromethyl)isoindolin-1-one |
| c) | 6-(5-azaspiro[2.4]heptan-5-ylmethyl)- |
| | 2-(3-((1R,2S)-1,2-difluoro-1-(4-methyl-4H- |
| | 1,2,4-triazol-3-yl)propan-2-yl)phenyl)-4- |
| | (trifluoromethyl)isoindolin-1-one |
| d) | 2-(3-(3-((R)-fluoro(4-methyl-4H-1,2,4- |
| | triazol-3-yl)methyl)oxetan-3-yl)phenyl)- |
| | 6-(((S)-3-methoxypyrrolidin-1-yl)methyl)-4- |
| | (trifluoromethyl)isoindolin-1-one |
| e) | 2-(3-(3-((S)-fluoro(4-methyl-4H- |
| | 1,2,4-triazol-3-yl)methyl)oxetan-3-yl)phenyl)- |
| | 6-(((S)-3-methoxypyrrolidin-1-yl)methyl)-4- |
| | (trifluoromethyl)isoindolin-1-one |
| f) | (R)-2-(3-(3-(fluoro(4-methyl-4H-1,2,4- |
| | triazol-3-yl)methyl)oxetan-3- |
| | yl)phenyl)-6-((4-fluoro-4- |
| | (hydroxymethyl)piperidin-1-yl)methyl)-4- |
| | (trifluoromethyl)isoindolin-1-one |
| g) | (S)-2-(3-(3-(fluoro(4-methyl-4H-1,2,4- |
| | triazol-3-yl)methyl)oxetan-3- |
| | yl)phenyl)-6-((4-fluoro-4- |
| | (hydroxymethyl)piperidin-1-yl)methyl)-4- |
| | (trifluoromethyl)isoindolin-1-one |
| h) | (R)-2-(3-(3-(fluoro(4-methyl-4H-1,2,4- |
| | triazol-3-yl)methyl)oxetan-3- |
| | yl)phenyl)-6-((3-fluoro-3- |
| | (methoxymethyl)azetidin-1-yl)methyl)-4- |
| | (trifluoromethyl)isoindolin-1-one |
| i) | (S)-2-(3-(3-(fluoro(4-methyl-4H-1,2,4- |
| | triazol-3-yl)methyl)oxetan-3- |
| | yl)phenyl)-6-((3-fluoro-3- |
| | (methoxymethyl)azetidin-1-yl)methyl)-4- |
| | (trifluoromethyl)isoindolin-1-one |
| j) | (R)-6-((3-(difluoromethyl)azetidin- |
| | 1-yl)methyl)-2-(3-(3-(fluoro(4-methyl-4H-1,2,4- |
| | triazol-3-yl)methyl)oxetan-3-yl)phenyl)-4- |
| | (trifluoromethyl)isoindolin-1-one |
| a) | (S)-6-((3-(difluoromethyl)azetidin-1- |
| | yl)methyl)-2-(3-(3-(fluoro(4-methyl-4H-1,2,4- |
| | triazol-3-yl)methyl)oxetan-3-yl)phenyl)-4- |
| | (trifluoromethyl)isoindolin-1-one |
| b) | (R)-6-(1-oxa-6-azaspiro[3.3]heptan-6- |
| | ylmethyl)-2-(3-(3-(fluoro(4-methyl- |
| | 4H-1,2,4-triazol-3-yl)methyl)oxetan-3-yl)phenyl)- |
| | 4-(trifluoromethyl)isoindolin-1-one |
| c) | (S)-6-(1-oxa-6-azaspiro[3.3]heptan-6- |
| | ylmethyl)-2-(3-(3-(fluoro(4-methyl- |
| | 4H-1,2,4-triazol-3-yl)methyl)oxetan-3-yl)phenyl)- |
| | 4-(trifluoromethyl)isoindolin-1-one |
| d) | (R)-2-(3-(3-(fluoro(4-methyl-4H-1,2,4- |
| | triazol-3-yl)methyl)oxetan-3-yl)phenyl)-6- |
| | ((4-hydroxy-4-methylpiperidin-1-yl)methyl)-4- |
| | (trifluoromethyl)isoindolin-1-one |
| e) | (S)-2-(3-(3-(fluoro(4-methyl-4H- |
| | 1,2,4-triazol-3-yl)methyl)oxetan-3-yl)phenyl)-6- |
| | ((4-hydroxy-4-methylpiperidin-1-yl)methyl)-4- |
| | (trifluoromethyl)isoindolin-1-one |
| f) | 2-(3-(3-((R)-fluoro(4-methyl-4H-1,2,4- |
| | triazol-3-yl)methyl)oxetan-3-yl)phenyl)-6- |
| | (((S)-3-(hydroxymethyl)piperidin-1-yl)methyl)-4- |
| | (trifluoromethyl)isoindolin-1-one |
| g) | 2-(3-(3-((S)-fluoro(4-methyl-4H-1,2,4- |
| | triazol-3-yl)methyl)oxetan-3-yl)phenyl)-6- |
| | (((S)-3-(hydroxymethyl)piperidin-1-yl)methyl)-4- |
| | (trifluoromethyl)isoindolin-1-one |
| h) | (R)-2-(3-(3-(fluoro(4-methyl-4H-1,2,4- |
| | triazol-3-yl)methyl)oxetan-3-yl)phenyl)-6- |
| | ((4-(methoxymethyl)piperidin-1-yl)methyl)-4- |
| | (trifluoromethyl)isoindolin-1-one |
| i) | (S)-2-(3-(3-(fluoro(4-methyl-4H-1,2,4- |
| | triazol-3-yl)methyl)oxetan-3-yl)phenyl)-6- |
| | ((4-(methoxymethyl)piperidin-1-yl)methyl)-4- |
| | (trifluoromethyl)isoindolin-1-one |
| j) | 2-(3-(3-((R)-fluoro(4-methyl-4H-1,2,4- |
| | triazol-3-yl)methyl)oxetan-3-yl)phenyl)-6- |
| | (((R)-2-(hydroxymethyl)pyrrolidin-1-yl)methyl)-4- |
| | (trifluoromethyl)isoindolin-1-one |
| a) | 2-(3-(3-((S)-fluoro(4-methyl-4H-1,2,4- |
| | triazol-3-yl)methyl)oxetan-3-yl)phenyl)-6- |
| | (((R)-2-(hydroxymethyl)pyrrolidin-1-yl)methyl)-4- |
| | (trifluoromethyl)isoindolin-1-one |
| b) | 2-(3-(3-((R)-fluoro(4-methyl-4H-1,2,4- |
| | triazol-3-yl)methyl)oxetan-3-yl)phenyl)-6- |
| | (((S)-2-(hydroxymethyl)pyrrolidin-1-yl)methyl)-4- |
| | (trifluoromethyl)isoindolin-1-one |
| c) | 2-(3-(3-((S)-fluoro(4-methyl-4H-1,2,4- |
| | triazol-3-yl)methyl)oxetan-3-yl)phenyl)-6- |
| | (((S)-2-(hydroxymethyl)pyrrolidin-1-yl)methyl)-4- |
| | (trifluoromethyl)isoindolin-1-one |
| d) | 2-(3-(3-((R)-fluoro(4-methyl-4H-1,2,4- |
| | triazol-3-yl)methyl)oxetan-3-yl)phenyl)-6- |
| | (((R)-3-(hydroxymethyl)piperidin-1-yl)methyl)-4- |
| | (trifluoromethyl)isoindolin-1-one |
| e) | 2-(3-(3-((S)-fluoro(4-methyl-4H-1,2,4- |
| | triazol-3-yl)methyl)oxetan-3-yl)phenyl)-6- |
| | (((R)-3-(hydroxymethyl)piperidin-1-yl)methyl)-4- |
| | (trifluoromethyl)isoindolin-1-one |
| f) | (R)-6-((1,1-difluoro-5-azaspiro[2.3]hexan- |
| | 5-yl)methyl)-2-(3-(3-(fluoro(4-methyl-4H-1,2,4- |
| | triazol-3-yl)methyl)oxetan-3-yl)phenyl)-4- |
| | (trifluoromethyl)isoindolin-1-one |
| g) | (S)-6-((1,1-difluoro-5-azaspiro[2.3]hexan- |
| | 5-yl)methyl)-2-(3-(3-(fluoro(4-methyl-4H- |
| | 1,2,4-triazol-3-yl)methyl)oxetan-3-yl)phenyl)-4- |
| | (trifluoromethyl)isoindolin-1-one |
| h) | 2-(3-(3-((R)-fluoro(4-methyl-4H- |
| | 1,2,4-triazol-3-yl)methyl)oxetan-3-yl)phenyl)-6- |
| | (((R)-2-(methoxymethyl)pyrrolidin-1-yl)methyl)-4- |
| | (trifluoromethyl)isoindolin-1-one |
| i) | 2-(3-(3-((S)-fluoro(4-methyl-4H-1,2,4- |
| | triazol-3-yl)methyl)oxetan-3-yl)phenyl)-6- |
| | (((R)-2-(methoxymethyl)pyrrolidin-1-yl)methyl)-4- |
| | (trifluoromethyl)isoindolin-1-one |
| j) | (R)-2-(3-(3-(fluoro(4-methyl-4H-1,2,4- |
| | triazol-3-yl)methyl)oxetan-3- |
| | yl)phenyl)-6-(((3-hydroxy-3- |
| | methylbutyl)(methyl)amino)methyl)-4- |
| | (trifluoromethyl)isoindolin-1-one |
| a) | (S)-2-(3-(3-(fluoro(4-methyl-4H-1,2,4- |
| | triazol-3-yl)methyl)oxetan-3- |
| | yl)phenyl)-6-(((3-hydroxy-3- |
| | methylbutyl)(methyl)amino)methyl)-4- |
| | (trifluoromethyl)isoindolin-1-one |
| b) | 2-(3-(3-((R)-fluoro(4-methyl-4H-1,2,4- |
| | triazol-3-yl)methyl)oxetan-3-yl)phenyl)-6-(((S)- |
| | 2-(methoxymethyl)pyrrolidin-1-yl)methyl)-4- |
| | (trifluoromethyl)isoindolin-1-one |
| c) | 2-(3-(3-((S)-fluoro(4-methyl-4H-1,2,4- |
| | triazol-3-yl)methyl)oxetan-3-yl)phenyl)-6-(((S)- |
| | 2-(methoxymethyl)pyrrolidin-1-yl)methyl)-4- |
| | (trifluoromethyl)isoindolin-1-one |
| d) | (R)-2-(3-(3-(fluoro(4-methyl-4H-1,2,4- |
| | triazol-3-yl)methyl)oxetan-3-yl)phenyl)- |
| | 6-((3-hydroxy-3-methylazetidin-1-yl)methyl)-4- |
| | (trifluoromethyl)isoindolin-1-one |
| e) | (S)-2-(3-(3-(fluoro(4-methyl-4H-1,2,4- |
| | triazol-3-yl)methyl)oxetan-3-yl)phenyl)- |
| | 6-((3-hydroxy-3-methylazetidin-1-yl)methyl)-4- |
| | (trifluoromethyl)isoindolin-1-one |
| f) | (R)-2-(3-(3-(fluoro(4-methyl-4H-1,2,4- |
| | triazol-3-yl)methyl)oxetan-3-yl)phenyl)- |
| | 6-((3-(methylsulfonyl)azetidin-1-yl)methyl)-4- |
| | (trifluoromethyl)isoindolin-1-one |
| g) | (S)-2-(3-(3-(fluoro(4-methyl-4H-1,2,4- |
| | triazol-3-yl)methyl)oxetan-3-yl)phenyl)-6- |
| | ((3-(methylsulfonyl)azetidin-1-yl)methyl)-4- |
| | (trifluoromethyl)isoindolin-1-one |
| h) | (R)-6-(2-azaspiro[3.3]heptan-2-ylmethyl)- |
| | 2-(3-(3-(fluoro(4-methyl-4H- |
| | 1,2,4-triazol-3-yl)methyl)oxetan-3-yl)phenyl)-4- |
| | (trifluoromethyl)isoindolin-1-one |
| i) | (S)-6-(2-azaspiro[3.3]heptan-2-ylmethyl)- |
| | 2-(3-(3-(fluoro(4-methyl-4H- |
| | 1,2,4-triazol-3-yl)methyl)oxetan-3-yl)phenyl)-4- |
| | (trifluoromethyl)isoindolin-1-one |
| j) | (R)-6-((3-ethyl-3-hydroxyazetidin-1- |
| | yl)methyl)-2-(3-(3-(fluoro(4-methyl-4H-1,2,4- |
| | triazol-3-yl)methyl)oxetan-3-yl)phenyl)-4- |
| | (trifluoromethyl)isoindolin-1-one |
| a) | (S)-6-((3-ethyl-3-hydroxyazetidin-1- |
| | yl)methyl)-2-(3-(3-(fluoro(4-methyl-4H-1,2,4- |
| | triazol-3-yl)methyl)oxetan-3-yl)phenyl)-4- |
| | (trifluoromethyl)isoindolin-1-one |
| b) | (R)-2-(3-(3-(fluoro(4-methyl-4H-1,2,4- |
| | triazol-3-yl)methyl)oxetan-3-yl)phenyl)-6- |
| | ((3-hydroxy-3-vinylazetidin-1-yl)methyl)-4- |
| | (trifluoromethyl)isoindolin-1-one |
| c) | (S)-2-(3-(3-(fluoro(4-methyl-4H-1,2,4- |
| | triazol-3-yl)methyl)oxetan-3-yl)phenyl)-6- |
| | ((3-hydroxy-3-vinylazetidin-1-yl)methyl)-4- |
| | (trifluoromethyl)isoindolin-1-one |
| d) | (R)-2-(3-(3-(fluoro(4-methyl-4H-1,2,4- |
| | triazol-3-yl)methyl)oxetan-3-yl)phenyl)-6- |
| | ((3-hydroxy-3-propylazetidin-1-yl)methyl)-4- |
| | (trifluoromethyl)isoindolin-1-one |
| e) | (S)-2-(3-(3-(fluoro(4-methyl-4H-1,2,4- |
| | triazol-3-yl)methyl)oxetan-3-yl)phenyl)-6- |
| | ((3-hydroxy-3-propylazetidin-1-yl)methyl)-4- |
| | (trifluoromethyl)isoindolin-1-one |
| f) | (R)-6-((3-cyclopropyl-3-hydroxyazetidin- |
| | 1-yl)methyl)-2-(3-(3-(fluoro(4-methyl-4H- |
| | 1,2,4-triazol-3-yl)methyl)oxetan-3-yl)phenyl)-4- |
| | (trifluoromethyl)isoindolin-1-one |
| g) | (S)-6-((3-cyclopropyl-3-hydroxyazetidin- |
| | 1-yl)methyl)-2-(3-(3-(fluoro(4-methyl-4H- |
| | 1,2,4-triazol-3-yl)methyl)oxetan-3-yl)phenyl)-4- |
| | (trifluoromethyl)isoindolin-1-one |
| h) | (R)-6-(6-oxa-1-azaspiro[3.3]heptan-1- |
| | ylmethyl)-2-(3-(3-(fluoro(4-methyl-4H-1,2,4- |
| | triazol-3-yl)methyl)oxetan-3-yl)phenyl)-4- |
| | (trifluoromethyl)isoindolin-1-one |
| i) | (S)-6-(6-oxa-1-azaspiro[3.3]heptan-1- |
| | ylmethyl)-2-(3-(3-(fluoro(4-methyl-4H-1,2,4- |
| | triazol-3-yl)methyl)oxetan-3-yl)phenyl)-4- |
| | (trifluoromethyl)isoindolin-1-one |
| j) | (R)-2-(3-(3-(fluoro(4-methyl-4H- |
| | 1,2,4-triazol-3-yl)methyl)oxetan-3-yl)phenyl)-6- |
| | ((3-(2-hydroxyethyl)azetidin-1-yl)methyl)-4- |
| | (trifluoromethyl)isoindolin-1-one |
| a) | (S)-2-(3-(3-(fluoro(4-methyl-4H- |
| | 1,2,4-triazol-3-yl)methyl)oxetan-3-yl)phenyl)- |
| | 6-((3-(2-hydroxyethyl)azetidin-1-yl)methyl)-4- |
| | (trifluoromethyl)isoindolin-1-one |
| b) | (R)-2-(3-(3-(fluoro(4-methyl-4H- |
| | 1,2,4-triazol-3-yl)methyl)oxetan-3-yl)phenyl)-6- |
| | ((3-methoxy-3-methylazetidin-1-yl)methyl)-4- |
| | (trifluoromethyl)isoindolin-1-one |
| c) | (S)-2-(3-(3-(fluoro(4-methyl-4H- |
| | 1,2,4-triazol-3-yl)methyl)oxetan-3- |
| | yl)phenyl)-6-((3-methoxy-3- |
| | methylazetidin-1-yl)methyl)-4- |
| | (trifluoromethyl)isoindolin-1-one |
| d) | (R)-6-((2,2-dioxido-2-thia-6- |
| | azaspiro[3.3]heptan-6-yl)methyl)-2-(3-(3- |
| | (fluoro(4-methyl-4H-1,2,4-triazol-3- |
| | yl)methyl)oxetan-3-yl)phenyl)-4- |
| | (trifluoromethyl)isoindolin-1-one |
| e) | (S)-6-((2,2-dioxido-2-thia-6- |
| | azaspiro[3.3]heptan-6-yl)methyl)-2-(3-(3- |
| | (fluoro(4-methyl-4H-1,2,4-triazol-3- |
| | yl)methyl)oxetan-3-yl)phenyl)-4- |
| | (trifluoromethyl)isoindolin-1-one |
| f) | (R)-2-(3-(3-(fluoro(4-methyl-4H-1,2,4- |
| | triazol-3-yl)methyl)oxetan-3-yl)phenyl)-6- |
| | ((3-hydroxy-3-isopropylazetidin-1-yl)methyl)-4- |
| | (trifluoromethyl)isoindolin-1-one |
| g) | (S)-2-(3-(3-(fluoro(4-methyl-4H-1,2,4- |
| | triazol-3-yl)methyl)oxetan-3-yl)phenyl)-6- |
| | ((3-hydroxy-3-isopropylazetidin-1-yl)methyl)-4- |
| | (trifluoromethyl)isoindolin-1-one |
| h) | (R)-2-(3-(3-(fluoro(4-methyl-4H-1,2,4- |
| | triazol-3-yl)methyl)oxetan-3- |
| | yl)phenyl)-6-((3-hydroxy-3-(methoxy- |
| | methyl)azetidin-1-yl)methyl)-4- |
| | (trifluoromethyl)isoindolin-1-one |
| i) | (S)-2-(3-(3-(fluoro(4-methyl-4H-1,2,4- |
| | triazol-3-yl)methyl)oxetan-3- |
| | yl)phenyl)-6-((3-hydroxy-3-(methoxy- |
| | methyl)azetidin-1-yl)methyl)-4- |
| | (trifluoromethyl)isoindolin-1-one |
| j) | 2-(3-(3-((R)-fluoro(4-methyl-4H-1,2,4- |
| | triazol-3-yl)methyl)oxetan-3- |
| | yl)phenyl)-6-(((S)-2-isopropyl-4- |
| | methylpiperazin-1-yl)methyl)-4- |
| | (trifluoromethyl)isoindolin-1-one |
| a) | 2-(3-(3-((S)-fluoro(4-methyl-4H-1,2,4- |
| | triazol-3-yl)methyl)oxetan-3- |
| | yl)phenyl)-6-(((S)-2-isopropyl-4- |
| | methylpiperazin-1-yl)methyl)-4- |
| | (trifluoromethyl)isoindolin-1-one |
| b) | 2-(3-(3,3-difluoro-1-((R)-fluoro(4- |
| | methyl-4H-1,2,4-triazol-3- |
| | yl)methyl)cyclobutyl)phenyl)-6-(((S)- |
| | 2-isopropyl-4-methylpiperazin-1- |
| | yl)methyl)-4-(trifluoromethyl)isoindolin-1-one |
| c) | 2-(3-(3,3-difluoro-1-((S)-fluoro(4- |
| | methyl-4H-1,2,4-triazol-3- |
| | yl)methyl)cyclobutyl)phenyl)-6-(((S)- |
| | 2-isopropyl-4-methylpiperazin-1- |
| | yl)methyl)-4-(trifluoromethyl)isoindolin-1-one |
| d) | 6-((R)-1-aminoethyl)-2-(3-(3-((R)- |
| | fluoro(4-methyl-4H-1,2,4-triazol-3- |
| | yl)methyl)oxetan-3-yl)phenyl)-4- |
| | (trifluoromethyl)isoindolin-1-one |
| e) | 6-((S)-1-aminoethyl)-2-(3-(3-((R)- |
| | fluoro(4-methyl-4H-1,2,4-triazol-3- |
| | yl)methyl)oxetan-3-yl)phenyl)-4- |
| | (trifluoromethyl)isoindolin-1-one |
| f) | 6-(((S)-4-acetyl-2-isopropylpiperazin- |
| | 1-yl)methyl)-2-(3-(3-((R)-fluoro(4-methyl-4H- |
| | 1,2,4-triazol-3-yl)methyl)oxetan-3-yl)phenyl)-4- |
| | (trifluoromethyl)isoindolin-1-one |
| g) | 6-(((S)-4-acetyl-2-isopropylpiperazin-1- |
| | yl)methyl)-2-(3-(3-((S)-fluoro(4-methyl-4H- |
| | 1,2,4-triazol-3-yl)methyl)oxetan-3-yl)phenyl)-4- |
| | (trifluoromethyl)isoindolin-1-one |
| h) | 2-(3-(3-((R)-fluoro(4-methyl-4H- |
| | 1,2,4-triazol-3-yl)methyl)oxetan-3- |
| | yl)phenyl)-6-(((S)-2-isopropyl-4- |
| | (methylsulfonyl)piperazin-1-yl)methyl)- |
| | 4-(trifluoromethyl)isoindolin-1-one |
| i) | 2-(3-(3-((S)-fluoro(4-methyl-4H-1,2,4- |
| | triazol-3-yl)methyl)oxetan-3- |
| | yl)phenyl)-6-(((S)-2-isopropyl-4- |
| | (methylsulfonyl)piperazin-1-yl)methyl)- |
| | 4-(trifluoromethyl)isoindolin-1-one |
| j) | 2-(3-(3-((R)-fluoro(4-methyl-4H-1,2,4- |
| | triazol-3-yl)methyl)oxetan-3- |
| | yl)phenyl)-6-(((S)-4-(2-fluoroethyl)- |
| | 2-isopropylpiperazin-1-yl)methyl)-4- |
| | (trifluoromethyl)isoindolin-1-one |
| k) | 2-(3-(3-((S)-fluoro(4-methyl-4H-1,2,4- |
| | triazol-3-yl)methyl)oxetan-3- |
| | yl)phenyl)-6-(((S)-4-(2-fluoroethyl)- |
| | 2-isopropylpiperazin-1-yl)methyl)-4- |
| | (trifluoromethyl)isoindolin-1-one |
| a) | 2-(3-(3-((S)-fluoro(4-methyl-4H-1,2,4- |
| | triazol-3-yl)methyl)oxetan-3- |
| | yl)phenyl)-6-(((S)-2-isopropyl-4- |
| | (oxetan-3-yl)piperazin-1-yl)methyl)-4- |
| | (trifluoromethyl)isoindolin-1-one |
| b) | 2-(3-(3-((R)-fluoro(4-methyl-4H-1,2,4- |
| | triazol-3-yl)methyl)oxetan-3- |
| | yl)phenyl)-6-(((S)-2-isopropyl-4- |
| | (oxetan-3-yl)piperazin-1-yl)methyl)-4- |
| | (trifluoromethyl)isoindolin-1-one |
| c) | 2-(3-(3-((S)-fluoro(4-methyl-4H-1,2,4- |
| | triazol-3-yl)methyl)oxetan-3- |
| | yl)phenyl)-6-(((S)-2-isopropyl-4-(2,2,2- |
| | trifluoroethyl)piperazin-1- |
| | yl)methyl)-4-(trifluoromethyl)isoindolin-1-one |
| d) | 2-(3-(3-((R)-fluoro(4-methyl-4H-1,2,4- |
| | triazol-3-yl)methyl)oxetan-3- |
| | yl)phenyl)-6-(((S)-2-isopropyl-4-(2,2,2- |
| | trifluoroethyl)piperazin-1- |
| | yl)methyl)-4-(trifluoromethyl)isoindolin-1-one |
| e) | (R)-4-bromo-2-(3-(3-(fluoro(4-methyl- |
| | 4H-1,2,4-triazol-3-yl)methyl)oxetan-3- |
| | yl)phenyl)isoindolin-1-one |
| f) | (R)-4-chloro-2-(3-(3-(fluoro(4- |
| | methyl-4H-1,2,4-triazol-3-yl)methyl)oxetan- |
| | 3-yl)phenyl)isoindolin-1-one |
| g) | (R)-2-(3-(3-(fluoro(4-methyl-4H-1,2,4- |
| | triazol-3-yl)methyl)oxetan-3- |
| | yl)phenyl)-4-iodoisoindolin-1-one |
| h) | (R)-2-(3-(3-(fluoro(4-methyl-4H-1,2,4- |
| | triazol-3-yl)methyl)oxetan-3- |
| | yl)phenyl)-4-methoxyisoindolin-1-one |
| i) | (R)-2-(3-(3-(fluoro(4-methyl-4H-1,2,4- |
| | triazol-3-yl)methyl)oxetan-3- |
| | yl)phenyl)-4-isopropoxyisoindolin-1-one |
| j) | (R)-2-(3-(3-(fluoro(4-methyl-4H-1,2,4- |
| | triazol-3-yl)methyl)oxetan-3- |
| | yl)phenyl)-4-methylisoindolin-1-one |
| k) | R)-2-(3-(3-(fluoro(4-methyl-4H-1,2,4- |
| | triazol-3-yl)methyl)oxetan-3- |
| | yl)phenyl)-4-ethylisoindolin-1-one |
| l) | (R)-2-(3-(3-(fluoro(4-methyl-4H-1,2,4- |
| | triazol-3-yl)methyl)oxetan-3- |
| | yl)phenyl)-4-phenylisoindolin-1-one |
| m) | (R)-4-ethynyl-2-(3-(3-(fluoro(4- |
| | methyl-4H-1,2,4-triazol-3- |
| | yl)methyl)oxetan-3-yl)phenyl)isoindolin-1-one |
| a) | (R)-2-(3-(3-(fluoro(4-methyl-4H-1,2,4- |
| | triazol-3-yl)methyl)oxetan-3- |
| | yl)phenyl)-6-((methylamino)methyl)- |
| | 4-(trifluoromethyl)isoindolin-1-one |
| b) | (R)-6-((ethylamino)methyl)-2-(3-(3- |
| | (fluoro(4-methyl-4H-1,2,4-triazol-3- |
| | yl)methyl)oxetan-3-yl)phenyl)-4- |
| | (trifluoromethyl)isoindolin-1-one |
| c) | (S)-2-(3-(3,3-difluoro-1-((4-methyl-4H- |
| | 1,2,4-triazol-3-yl)methyl)cyclobutyl)phenyl)- |
| | 6-((2,4-dimethylpiperazin-1-yl)methyl)-4- |
| | (trifluoromethyl)isoindolin-1-one |
| d) | (S)-2-(3-(3,3-difluoro-1-((4- |
| | methyl-4H-1,2,4-triazol-3- |
| | yl)methyl)cyclobutyl)phenyl)-6-((2- |
| | isopropyl-4-methylpiperazin-1- |
| | yl)methyl)-4-(trifluoromethyl)isoindolin-1-one |
| e) | 2-(3-(1-((R)-fluoro(4-methyl-4H-1,2,4-triazol-3- |
| | yl)methyl)cyclobutyl)phenyl)-6-(((S)- |
| | 2-isopropylpiperazin-1-yl)methyl)-4- |
| | (trifluoromethyl)isoindolin-1-one |
| f) | 2-(3-(1-((S)-fluoro(4-methyl-4H-1,2,4-triazol-3- |
| | yl)methyl)cyclobutyl)phenyl)-6-(((S)-2- |
| | isopropylpiperazin-1-yl)methyl)-4- |
| | (trifluoromethyl)isoindolin-1-one |
| g) | 2-(3-(3-((R)-fluoro(4-methyl-4H-1,2,4- |
| | triazol-3-yl)methyl)oxetan-3- |
| | yl)phenyl)-6-(((R)-3-isopropyl-4- |
| | methylpiperazin-1-yl)methyl)-4- |
| | (trifluoromethyl)isoindolin-1-one |
| h) | (S)-2-(3-(3,3-difluoro-1-((4- |
| | methyl-4H-1,2,4-triazol-3- |
| | yl)methyl)cyclobutyl)phenyl)-6-((2- |
| | isopropylpiperazin-1-yl)methyl)-4- |
| | (trifluoromethyl)isoindolin-1-one |
| i) | (R)-2-(3-(3,3-difluoro-1- |
| | ((4-methyl-4H-1,2,4-triazol-3- |
| | yl)methyl)cyclobutyl)phenyl)-6-((3,4- |
| | dimethylpiperazin-1-yl)methyl)-4- |
| | (trifluoromethyl)isoindolin-1-one |
| j) | (S)-2-(3-(1-(Difluoro(4-methyl-4H-1,2,4- |
| | triazol-3-yl)methyl)cyclopropyl)phenyl)-6-((2- |
| | isopropyl-4-methylpiperazin-1- |
| | yl)methyl)-4-(trifluoromethyl)isoindolin-1-one |
| k) | (S)-6-((2-Isopropyl-4-methylpiperazin- |
| | 1-yl)methyl)-2-(3-(3-((5-methyl-1H-1,2,3- |
| | triazol-1-yl)methyl)oxetan-3-yl)phenyl)-4- |
| | (trifluoromethyl)isoindolin-1-one |
| a) | (R)-2-(3-(3-(fluoro(4-methyl-4H-1,2,4- |
| | triazol-3-yl)methyl)oxetan-3- |
| | yl)phenyl)-6-((isopropylamino)methyl)- |
| | 4-(trifluoromethyl)isoindolin-1- |
| | one formate |
| b) | (R)-6-((Cyclobutylamino)methyl)-2-(3- |
| | (3-(fluoro(4-methyl-4H-1,2,4- |
| | triazol-3-yl)methyl)oxetan-3-yl)phenyl)- |
| | 4-(trifluoromethyl)isoindolin-1- |
| | one formate |
| c) | (R)-2-(3-(3-(fluoro(4-methyl-4H-1,2,4- |
| | triazol-3-yl)methyl)oxetan-3- |
| | yl)phenyl)-6-((propylamino)methyl)- |
| | 4-(trifluoromethyl)isoindolin-1-one |
| | formate |
| d) | (R)-2-(3-(3-(fluoro(4-methyl-4H-1,2,4- |
| | triazol-3-yl)methyl)oxetan-3-yl)phenyl)- |
| | 6-(((1-methylcyclopropyl)amino)methyl)- |
| | 4-(trifluoromethyl)isoindolin-1-one |
| e) | (R)-2-(3-(3-(fluoro(4-methyl-4H-1,2,4- |
| | triazol-3-yl)methyl)oxetan-3-yl)phenyl)- |
| | 6-(((1-methylcyclobutyl)amino)methyl)-4- |
| | (trifluoromethyl)isoindolin-1-one |
| f) | (R)-2-(3-(3-(fluoro(4-methyl-4H-1,2,4- |
| | triazol-3-yl)methyl)oxetan-3- |
| | yl)phenyl)-6-(((3-fluoropropyl)amino)methyl)-4- |
| | (trifluoromethyl)isoindolin-1-one |
| g) | (2-(3-(3,3-difluoro-1-((4-methyl-4H- |
| | 1,2,4-triazol-3-yl)methyl)-cyclobutyl)phenyl)- |
| | 6-(((1-methylcyclobutyl)amino)methyl)-4- |
| | (trifluoromethyl)isoindolin-1-one) |
| h) | 2-(4-(1-((R)-fluoro(4-methyl-4H-1,2,4- |
| | triazol-3-yl)methyl)cyclobutyl)-6- |
| | ((2-hydroxyethyl)amino)pyridin-2-yl)- |
| | 6-(((S)-2-isopropyl-4-methyl-piperazin-1- |
| | yl)methyl)-4-(trifluoromethyl)isoindolin-1-one |
| i) | 2-(4-(1-((S)-fluoro(4-methyl-4H-1,2,4- |
| | triazol-3-yl)methyl)cyclobutyl)-6- |
| | ((2-hydroxyethyl)amino)pyridin- |
| | 2-yl)-6-(((S)-2-isopropyl-4- |
| | methylpiperazin-1-yl)methyl)-4- |
| | (trifluoromethyl)isoindolin-1-one |
| j) | 2-(3-(1-(difluoro(4-methyl-4H-1,2,4- |
| | triazol-3-yl)methyl)cyclobutyl)-phenyl)- |
| | 6-(((1-methylcyclobutyl)amino)methyl)-4- |
| | (trifluoromethyl)isoindolin-1-one |
| a) | (S)-2-(4-(3,3-difluoro-1-((4-methyl- |
| | 4H-1,2,4-triazol-3-yl)methyl)-cyclobutyl)- |
| | 6-(ethylamino)pyridin-2-yl)-6-((2-isopropyl-4- |
| | methylpiperazin-1-yl)methyl)-4- |
| | (trifluoromethyl)isoindolin-1-one formate |
| b) | 2-(3-(3-(fluoro(4-methyl-1H-pyrazol- |
| | 5-yl)methyl)oxetan-3-yl)phenyl)-6-(((S)-2- |
| | isopropyl-4-methylpiperazin-1-yl)methyl)-4- |
| | (trifluoromethyl)isoindolin-1-one |
| c) | 2-(6-((cyclopropylmethyl)amino)-4- |
| | (1-((4-methyl-4H-1,2,4-triazol-3- |
| | yl)methyl)cyclobutyl)pyridin-2-yl)-6- |
| | (((1-methylcyclobutyl)amino)methyl)- |
| | 4-(trifluoromethyl)isoindolin-1-one |
| d) | (S)-N-(6-(6-((2-isopropyl-4- |
| | methylpiperazin-1-yl)methyl)-1-oxo-4- |
| | (trifluoromethyl)isoindolin-2-yl)-4- |
| | (1-((4-methyl-4H-1,2,4-triazol-3- |
| | yl)methyl)cyclobutyl)pyridin-2-yl)acrylamide |
| e) | (S)-2-(3-(3,3-difluoro-1-((4-methyl- |
| | 4H-1,2,4-triazol-3-yl)methyl)- |
| | cyclobutyl)phenyl)-6-((2-isopropyl-4- |
| | methylpiperazin-1-yl)methyl)-4- |
| | (trifluoromethyl)isoindolin-1-one |
| f) | 2-(6-benzyl-4-(1-((4-methyl-4H-1,2,4- |
| | triazol-3-yl)methyl)cyclobutyl)-pyridin- |
| | 2-yl)-6-(((1-methylcyclobutyl)amino)methyl)-4- |
| | (trifluoromethyl)isoindolin-1-one |
| g) | 2-(3-(3-((4-methyl-4H-1,2,4-triazol-3- |
| | yl)methyl)-1,1-dioxidothietan-3-yl)phenyl)-6- |
| | (((1-methylcyclobutyl)amino)methyl)- |
| | 4-(trifluoromethyl)-isoindolin-1-one |
| h) | 2-(3-(3-((5-methyl-1H-1,2,3-triazol-1- |
| | yl)methyl)oxetan-3-yl)phenyl)-6- |
| | (((1-methylcyclobutyl)amino)methyl)-4- |
| | (trifluoromethyl)isoindolin-1-one |
| i) | 2-(3-(3,3-difluoro-1-((4-methyl-4H-1,2,4- |
| | triazol-3-yl)methyl)cyclobutyl)-phenyl)-5- |
| | fluoro-6-(((1-methylcyclobutyl)amino)methyl)-4- |
| | (trifluoromethyl)isoindolin-1-one |
| j) | (R)-2-(3-(3,3-difluoro-1-((4-methyl-4H-1,2,4- |
| | triazol-3-yl)methyl)cyclobutyl)phenyl)-6-(1- |
| | ((1-methylcyclobutyl)amino)ethyl)-4- |
| | (trifluoromethyl)isoindolin-1-one) |
| a) | ((S)-2-(3-(3,3-difluoro-1-((4-methyl-4H-1,2,4- |
| | triazol-3-yl)methyl)cyclobutyl)phenyl)-6-(1- |
| | ((1-methylcyclobutyl)amino)ethyl)-4- |
| | (trifluoromethyl)isoindolin-1-one |
| b) | 2-(3-(3-((R)-fluoro(4-methyl-4H-1,2,4- |
| | triazol-3-yl)methyl)oxetan-3-yl)phenyl)- |
| | 6-((R)-1-((1-methylcyclobutyl)amino)ethyl)-4- |
| | (trifluoromethyl)isoindolin-1-one |
| c) | (2-(3-(3-((R)-fluoro(4-methyl-4H-1,2,4- |
| | triazol-3-yl)methyl)oxetan-3-yl)phenyl)-6- |
| | ((S)-1-((1-methylcyclobutyl)amino)ethyl)-4- |
| | (trifluoromethyl)isoindolin-1-one |
| d) | 2-(3-(3-((R)-fluoro(4-methyl-4H-1,2,4- |
| | triazol-3-yl)methyl)oxetan-3-yl)phenyl)-6- |
| | ((R)-1-((1-methylcyclobutyl)amino)ethyl)-4- |
| | (trifluoromethyl)isoindolin-1-one) |
| e) | (2-(3-(3-((R)-fluoro(4-methyl-4H-1,2,4- |
| | triazol-3-yl)methyl)oxetan-3-yl)phenyl)-6- |
| | ((S)-1-((1-methylcyclobutyl)amino)ethyl)-4- |
| | (trifluoromethyl)isoindolin-1-one |
| f) | (R)-4-(difluoromethyl)-2-(3-(3- |
| | (fluoro(4-methyl-4H-1,2,4-triazol-3- |
| | yl)methyl)oxetan-3-yl)phenyl)-6-(((1- |
| | methylcyclobutyl)amino)methyl)isoindolin- |
| | 1-one formate |
| g) | 2-(3-(3,3-difluoro-1-((4-methyl-4H-1,2,4- |
| | triazol-3-yl)methyl)-^{,}cyclo-^{,}butyl)phenyl)- |
| | 4-(difluoromethyl)-6-(((1- |
| | methylcyclobutyl)amino)methyl)isoindolin-1-one |
| h) | 2-(4-(1-((4-methyl-4H-1,2,4-triazol- |
| | 3-yl)methyl)cyclobutyl)-6- |
| | (methylthio)pyridin-2-yl)-6-(((1- |
| | methylcyclobutyl)amino)methyl)-4- |
| | (trifluoromethyl)isoindolin-1-one |
| i) | 4-chloro-2-(3-(3,3-difluoro-1-((4- |
| | methyl-4H-1,2,4-triazol-3- |
| | yl)-methyl)¬cyclobutyl)¬phenyl)-6-(((1- |
| | methylcyclobutyl)amino)methyl)isoindolin- |
| | 1-one formate |
| j) | 2-(3-(1-(difluoro(4-methyl-4H-1,2,4- |
| | triazol-3-yl)methyl)-3,3- |
| | difluoro-Oyclobutyl)phenyl)-4- |
| | (trifluoromethyl)isoindolin-1-one |
| a) | (S)-6-((2-isopropyl-4-methylpiperazin- |
| | 1-yl)methyl)-2-(3-(3-((5-methyl-1H-1,2,4- |
| | triazol-1-yl)methyl)oxetan-3-yl)phenyl)-4- |
| | (trifluoromethyl)isoindolin-1-one |
| b) | (S)-6-((2-isopropyl-4-methylpiperazin- |
| | 1-yl)methyl)-2-(3-(3-((3-methyl-1H-1,2,4- |
| | triazol-1-yl)methyl)oxetan-3-yl)phenyl)-4- |
| | (trifluoromethyl)isoindolin-1-one |
| c) | (S)-6-((2-isopropyl-4-methylpiperazin- |
| | 1-yl)methyl)-2-(3-(3-((4-methyl-2H-1,2,3- |
| | triazol-2-yl)methyl)oxetan-3-yl)phenyl)-4- |
| | (trifluoromethyl)isoindolin-1-one) |
| d) | ((S)-6-((2-isopropyl-4-methylpiperazin- |
| | 1-yl)methyl)-2-(3-(3-((4-methyl-1H-1,2,3- |
| | triazol-1-yl)methyl)oxetan-3-yl)phenyl)-4- |
| | (trifluoromethyl)isoindolin-1-one) |
| e) | ((S)-6-((2-isopropyl-4-methylpiperazin- |
| | 1-yl)methyl)-2-(3-(3-((5-methyl-1H-1,2,3- |
| | triazol-1-yl)methyl)-Oxetan-3-yl)phenyl)-4- |
| | (trifluoromethyl)isoindolin-1-one |
| f) | 2-(3-(3-((R)-fluoro(4-phenyl-4H-1,2,4- |
| | triazol-3-yl)methyl)oxetan-3- |
| | yl)phenyl)-6-(((S)-2-isopropyl-4- |
| | methylpiperazin-1-yl)methyl)-4- |
| | (trifluoromethyl)isoindolin-1-one) |
| g) | (2-(3-(3-((S)-fluoro(4-phenyl-4H-1,2,4- |
| | triazol-3-yl)methyl)oxetan-3-yl)- |
| | phenyl)-6-(((S)-2-isopropyl-4- |
| | methylpiperazin-1-yl)methyl)-4- |
| | (trifluoromethyl)isoindolin-1-one |
| h) | 2-(3-((S)-2-((S)-fluoro(4-methyl-4H- |
| | 1,2,4-triazol-3-yl)methyl)oxetan-2- |
| | yl)phenyl)-6-(((S)-2-isopropyl-4- |
| | methylpiperazin-1-yl)methyl)-4- |
| | (trifluoromethyl)isoindolin-1-one |
| i) | 2-(3-((S)-2-((R)-fluoro(4-methyl-4H- |
| | 1,2,4-triazol-3-yl)methyl)oxetan-2- |
| | yl)phenyl)-6-(((S)-2-isopropyl-4- |
| | methylpiperazin-1-yl)methyl)-4- |
| | (trifluoromethyl)isoindolin-1-one |
| j) | (S)-6-((2-isopropyl-4-methylpiperazin- |
| | 1-yl)methyl)-2-(3-(3-((5-methyl- |
| | 1H-pyrazol-1-yl)methyl)oxetan-3-yl)phenyl)- |
| | 4-(trifluoromethyl)isoindolin-1-one |
| a) | (S)-6-((2-isopropyl-4-methylpiperazin- |
| | 1-yl)methyl)-2-(3-(3-((3-methyl- |
| | 1H-pyrazol-1-yl)methyl)oxetan-3- |
| | yl)phenyl)-4-(trifluoromethyl)isoindolin- |
| | 1-one |
| b) | 2-(6-cyclobutyl-4-(3-((4-methyl-4H-1,2,4- |
| | triazol-3-yl)methyl)oxetan-3-yl)pyridin- |
| | 2-yl)-4-(trifluoromethyl)isoindolin-1-one |
| c) | (S)-2-(3-(3-((4-(difluoromethyl)-4H- |
| | 1,2,4-triazol-3-yl)methyl)oxetan-3- |
| | yl)phenyl)-6-((2-isopropyl-4- |
| | methylpiperazin-1-yl)methyl)-4- |
| | (trifluoromethyl)isoindolin-1-one |
| d) | 2-(3-(3-((R)-(4-(difluoromethyl)-4H-1,2,4- |
| | triazol-3-yl)fluoromethyl)oxetan-3- |
| | yl)phenyl)-6-(((S)-2-isopropyl-4- |
| | methylpiperazin-1-yl)methyl)-4- |
| | (trifluoromethyl)isoindolin-1-one) |
| e) | 2-(3-(3-((S)-(4-(difluoromethyl)-4H-1,2,4- |
| | triazol-3-yl)fluoromethyl)oxetan-3- |
| | yl)phenyl)-6-(((S)-2-isopropyl-4- |
| | methylpiperazin-1-yl)methyl)-4- |
| | (trifluoromethyl)isoindolin-1-one |
| f) | 2-(3-(3-((R)-fluoro(4-fluoro-1-methyl- |
| | 1H-imidazol-2-yl)methyl)oxetan-3- |
| | yl)phenyl)-6-(((S)-2-isopropyl-4- |
| | methylpiperazin-1-yl)methyl)-4- |
| | (trifluoromethyl)isoindolin-1-one) |
| g) | 2-(3-(3-((S)-fluoro(4-fluoro-1-methyl- |
| | 1H-imidazol-2-yl)methyl)oxetan-3- |
| | yl)phenyl)-6-(((S)-2-isopropyl-4- |
| | methylpiperazin-1-yl)methyl)-4- |
| | (trifluoromethyl)isoindolin-1-one |
| h) | (S)-2-(3-(3-((4-ethyl-4H-1,2,4-triazol- |
| | 3-yl)fluoromethyl)oxetan-3- |
| | yl)phenyl)-4-(trifluoromethyl)isoindolin-1-one) |
| i) | (R)-2-(3-(3-((4-ethyl-4H-1,2,4- |
| | triazol-3-yl)fluoromethyl)oxetan-3- |
| | yl)phenyl)-4-(trifluoromethyl)isoindolin-1-one |
| j) | 2-(3-(3,3-difluoro-1-((4-methyl-4H- |
| | 1,2,4-triazol-3-yl)methyl)- |
| | cyclobutyl)phenyl)-6-((2S,6S)-6-ethylpiperidin- |
| | 2-yl)-4-(trifluoromethyl)isoindolin-1-one) |
| k) | 2-(3-(3,3-difluoro-1-((4-methyl-4H-1,2,4-triazol-3- |
| | yl)methyl)cyclobutyl)phenyl)-6- |
| | ((2R,6R)-6-ethylpiperidin-2-yl)-4- |
| | (trifluoromethyl)isoindolin-1-one |
| a) | (S)-2-(6-(allylamino)-4-(1-((4- |
| | methyl-4H-1,2,4-triazol-3- |
| | yl)methyl)cyclobutyl)pyridin-2-yl)-6- |
| | ((2-isopropyl-4-methylpiperazin-1- |
| | yl)methyl)-4-(trifluoromethyl)isoindolin-1-one |
| b) | 2-(3-(3,3-difluoro-1-((4-methyl-4H-1,2,4-triazol-3- |
| | yl)methyl)cyclobutyl)phenyl)-5-fluoro-6-(((1- |
| | methylcyclobutyl)amino)methyl)-4- |
| | (trifluoromethyl)isoindolin-1-one |
| c) | 2-(6-(cyclopentylamino)-4-(3-((4- |
| | methyl-4H-1,2,4-triazol-3- |
| | yl)methyl)oxetan-3-yl)pyridin-2-yl)- |
| | 6-((methylamino)methyl)-4- |
| | (trifluoromethyl)isoindolin-1-one |
| d) | 2-(6-((cyclopropylmethyl)amino)-4- |
| | (1-((R)-fluoro(4-methyl-4H-1,2,4- |
| | triazol-3-yl)methyl)cyclobutyl)pyridin- |
| | 2-yl)-6-(((S)-2-isopropyl-4- |
| | methylpiperazin-1-yl)methyl)-4- |
| | (trifluoromethyl)isoindolin-1-one |
| e) | 2-(6-((cyclopropylmethyl)amino)- |
| | 4-(1-((S)-fluoro(4-methyl-4H-1,2,4- |
| | triazol-3-yl)methyl)cyclobutyl)pyridin- |
| | 2-yl)-6-(((S)-2-isopropyl-4- |
| | methylpiperazin-1-yl)methyl)-4- |
| | (trifluoromethyl)isoindolin-1-one |
| f) | (S)-2-(4-(3,3-difluoro-1-((4-methyl-4H-1,2,4-triazol- |
| | 3-yl)methyl)cyclobutyl)-6-(dimethylamino)pyridin- |
| | 2-yl)-6-((2-isopropyl-4-methylpiperazin-1- |
| | yl)methyl)-4-(trifluoromethyl)isoindolin-1-one |
| g) | (S)-4-chloro-2-(3-(3,3-difluoro-1- |
| | ((4-methyl-4H-1,2,4-triazol-3- |
| | yl)methyl)cyclobutyl)phenyl)-6-((2- |
| | isopropyl-4-methylpiperazin-1- |
| | yl)methyl)isoindolin-1-one |
| h) | 4-chloro-2-(3-(3,3-difluoro-1-((4- |
| | methyl-4H-1,2,4-triazol-3- |
| | yl)methyl)cyclobutyl)phenyl)-6-(((1- |
| | methylcyclobutyl)amino)methyl)isoindolin- |
| | 1-one formate |
| i) | 6-((tert-butylamino)methyl)-2-(3-(3,3- |
| | difluoro-1-((4-methyl-4H-1,2,4- |
| | triazol-3-yl)methyl)cyclobutyl)phenyl)- |
| | 4-(trifluoromethyl)isoindolin-1-one |
| j) | 2-(6-cyclopropyl-4-(1-((4-methyl-4H-1,2,4-triazol- |
| | 3-yl)methyl)cyclobutyl)pyridin-2-yl)-6-(((1- |
| | methylcyclobutyl)amino)methyl)- |
| | 4-(trifluoromethyl)isoindolin-1-one |
| a) | 2-(6-ethoxy-4-(1-((4-methyl-4H-1,2,4-triazol-3- |
| | yl)methyl)cyclobutyl)pyridin-2-yl)-6-(((1- |
| | methylcyclobutyl)amino)methyl)- |
| | 4-(trifluoromethyl)isoindolin-1-one |
| b) | 2-(6-(ethylthio)-4-(1-((4-methyl-4H- |
| | 1,2,4-triazol-3-yl)methyl)- |
| | cyclobutyl)pyridin-2-yl)-6-(((1- |
| | methylcyclobutyl)amino)methyl)-4- |
| | (trifluoromethyl)isoindolin-1-one |
| c) | 2-(3-(3-(difluoro(4-methyl-4H-1,2,4- |
| | triazol-3-yl)methyl)oxetan-3- |
| | yl)phenyl)-6-(((1-methylcyclobutyl)amino)methyl)- |
| | 4-(trifluoromethyl)isoindolin-1-one |
| d) | 2-(3-(3,3-difluoro-1-((4-methyl-4H-1,2,4-triazol-3- |
| | yl)methyl)cyclobutyl)phenyl)-4-(difluoromethyl)-6- |
| | (((1-methylcyclobutyl)amino)methyl)isoindolin- |
| | 1-one |
| e) | (R)-4-(difluoromethyl)-2-(3-(3- |
| | (fluoro(4-methyl-4H-1,2,4-triazol-3- |
| | yl)methyl)oxetan-3-yl)phenyl)-6-(((1- |
| | methylcyclobutyl)amino)methyl)isoindolin- |
| | 1-one formate |
| f) | 2-(6-(ethylamino)-4-(3-((4-methyl-4H- |
| | 1,2,4-triazol-3-yl)methyl)oxetan-3-yl)pyridin- |
| | 2-yl)-6-(((1-methylcyclobutyl)amino)methyl)-4- |
| | (trifluoromethyl)isoindolin-1-one |
| g) | (S)-2-(6-(ethylamino)-4-(3-((4- |
| | methyl-4H-1,2,4-triazol-3- |
| | yl)methyl)oxetan-3-yl)pyridin-2-yl)-6- |
| | ((2-isopropyl-4-methylpiperazin-1- |
| | yl)methyl)-4-(trifluoromethyl)isoindolin-1-one |
| h) | 2-(6-(cyclopentylmethyl)-4-(3- |
| | ((4-methyl-4H-1,2,4-triazol-3- |
| | yl)methyl)oxetan-3-yl)pyridin-2-yl)- |
| | 4-(trifluoromethyl)isoindolin-1-one |
| i) | (S)-2-(6-(cyclopentyloxy)-4-(3- |
| | ((4-methyl-4H-1,2,4-triazol-3- |
| | yl)methyl)oxetan-3-yl)pyridin-2-yl)-6- |
| | ((2-isopropyl-4-methylpiperazin-1- |
| | yl)methyl)-4-(trifluoromethyl)isoindolin-1-one |
| j) | 2-(6-(cyclopentyloxy)-4-(3-((4- |
| | methyl-4H-1,2,4-triazol-3- |
| | yl)methyl)oxetan-3-yl)pyridin-2-yl)-6-(((1- |
| | methylcyclobutyl)amino)methyl)- |
| | 4-(trifluoromethyl)isoindolin-1-one |
| a) | 2-(6-(cyclopentylamino)-4-(1- |
| | ((4-methyl-4H-1,2,4-triazol-3- |
| | yl)methyl)cyclobutyl)pyridin-2-yl)-6-(((1- |
| | methylcyclobutyl)amino)methyl)- |
| | 4-(trifluoromethyl)isoindolin-1-one |
| b) | 2-(6-(cyclopentylamino)-4-(1- |
| | ((4-methyl-4H-1,2,4-triazol-3- |
| | yl)methyl)cyclobutyl)pyridin-2-yl)-4- |
| | (trifluoromethyl)isoindolin-1-one |
| c) | (S)-2-(6-(cyclopentylamino)-4-(3- |
| | ((4-methyl-4H-1,2,4-triazol-3- |
| | yl)methyl)oxetan-3-yl)pyridin-2-yl)- |
| | 6-((2-isopropyl-4-methylpiperazin-1-yl)methyl)- |
| | 4-(trifluoromethyl)isoindolin-1-one formate |
| d) | 2-(6-(cyclopentylamino)-4-(3- |
| | ((4-methyl-4H-1,2,4-triazol-3- |
| | yl)methyl)oxetan-3-yl)pyridin-2-yl)-6-(((1- |
| | methylcyclobutyl)amino)methyl)-4- |
| | (trifluoromethyl)isoindolin-1-one |
| e) | (S)-6-(6-((2-isopropyl-4-methyl |
| | piperazin-1-yl)methyl)-1-oxo-4- |
| | (trifluoromethyl)isoindolin-2-yl)-4- |
| | (1-((4-methyl-4H-1,2,4-triazol-3- |
| | yl)methyl)cyclobutyl)picolinonitrile |
| f) | 2-(6-cyclopropyl-4-(3-((4-methyl-4H- |
| | 1,2,4-triazol-3-yl)methyl)oxetan-3-yl)pyridin- |
| | 2-yl)-6-(((1-methylcyclobutyl)amino)methyl)-4- |
| | (trifluoromethyl)isoindolin-1-one |
| g) | 2-(3-(3-((4-methyl-4H-1,2,4-triazol-3- |
| | yl)methyl)oxetan-3-yl)phenyl)-6- |
| | (((1-methylcyclobutyl)amino)methyl)- |
| | 4-(trifluoromethyl)isoindolin-1-one |
| h) | 2-(6-((1-(2-methoxyethyl)piperidin-4- |
| | yl)amino)-4-(1-((4-methyl-4H-1,2,4- |
| | triazol-3-yl)methyl)cyclobutyl)pyridin-2-yl)-6-(((1- |
| | methylcyclobutyl)amino)methyl)-4- |
| | (trifluoromethyl)isoindolin-1-one |
| i) | 2-(4-(1-((4-methyl-4H-1,2,4-triazol- |
| | 3-yl)methyl)cyclobutyl)-6-((3- |
| | morpholinopropyl)amino)pyridin-2-yl)-6-(((1- |
| | methylcyclobutyl)amino)methyl)-4- |
| | (trifluoromethyl)isoindolin-1-one |
| j) | 2-(6-ethoxy-4-(3-((4-methyl-4H-1,2,4- |
| | triazol-3-yl)methyl)oxetan-3-yl)pyridin- |
| | 2-yl)-6-(((1-methylcyclobutyl)amino)methyl)-4- |
| | (trifluoromethyl)isoindolin-1-one |
| k) | 4-(difluoromethyl)-2-(3-(3- |
| | ((5-methyl-1H-1,2,3-triazol-1- |
| | yl)methyl)oxetan-3-yl)phenyl)-6-(((1- |
| | methylcyclobutyl)amino)methyl)isoindolin-1-one |
| a) | 2-(6-(((1S,4S)-4-aminocyclohexyl)amino)- |
| | 4-(1-((4-methyl-4H-1,2,4- |
| | triazol-3-yl)methyl)cyclobutyl)pyridin-2-yl)-6-(((1- |
| | methylcyclobutyl)amino)methyl)-4- |
| | (trifluoromethyl)isoindolin-1-one |
| b) | N-ethyl-2-((4-(1-((4-methyl-4H-1,2,4- |
| | triazol-3-yl)methyl)cyclobutyl)-6-(6- |
| | (((1-methylcyclobutyl)amino)methyl)- |
| | 1-oxo-4-(trifluoromethyl)isoindolin- |
| | 2-yl)pyridin-2-yl)amino)acetamide formate |
| c) | 3-((4-(1-((4-methyl-4H-1,2,4-triazol-3- |
| | yl)methyl)cyclobutyl)-6-(6-(((1- |
| | methylcyclobutyl)amino)methyl)-1- |
| | oxo-4-(trifluoromethyl)isoindolin-2- |
| | yl)pyridin-2-yl)amino)propanenitrile |
| d) | 2-(4-(1-((4-methyl-4H-1,2,4-triazol- |
| | 3-yl)methyl)cyclobutyl)-6-((2-(4- |
| | methylpiperidin-1-yl)ethyl)amino)pyridin-2-yl)- |
| | 6-(((1-methylcyclobutyl)amino)methyl)-4- |
| | (trifluoromethyl)isoindolin-1-one |
| | formate |
| e) | 2-(4-(1-((4-methyl-4H-1,2,4-triazol- |
| | 3-yl)methyl)cyclobutyl)-6- |
| | (methylamino)pyridin-2-yl)-6-(((1- |
| | methylcyclobutyl)amino)methyl)-4- |
| | (trifluoromethyl)isoindolin-1-one formate |
| f) | N,N-dimethyl-2-((4-(1-((4-methyl-4H-1,2,4- |
| | triazol-3-yl)methyl)cyclobutyl)-6-(6-(((1- |
| | methylcyclobutyl)amino)methyl)-1-oxo-4- |
| | (trifluoromethyl)isoindolin-2-yl)pyridin-2- |
| | yl)amino)acetamide formate |
| g) | N-methyl-2-((4-(1-((4-methyl-4H-1,2,4- |
| | triazol-3-yl)methyl)cyclobutyl)-6- |
| | (6-(((1-methylcyclobutyl)amino)methyl)-1-oxo-4- |
| | (trifluoromethyl)isoindolin-2-yl)pyridin- |
| | 2-yl)amino)ethanesulfonamide |
| h) | 2-(6-(((1-methyl-1H-pyrazol-4- |
| | yl)methyl)amino)-4-(1-((4-methyl-4H-1,2,4- |
| | triazol-3-yl)methyl)cyclobutyl)pyridin-2-yl)-6-(((1- |
| | methylcyclobutyl)amino)methyl)-4- |
| | (trifluoromethyl)isoindolin-1-one |
| i) | N-(4-((4-(1-((4-methyl-4H-1,2,4-triazol- |
| | 3-yl)methyl)cyclobutyl)-6-(6-(((1- |
| | methylcyclobutyl)amino)methyl)-1- |
| | oxo-4-(trifluoromethyl)isoindolin-2- |
| | yl)pyridin-2-yl)amino)butyl)acetamide formate |
| j) | 2-(6-(((1,1-dioxidotetrahydro-2H-thiopyran- |
| | 4-yl)methyl)amino)-4-(1-((4- |
| | methyl-4H-1,2,4-triazol-3-yl)methyl)cyclo- |
| | butyl)pyridin-2-yl)-6-(((1- |
| | methylcyclobutyl)amino)methyl)-4- |
| | (trifluoromethyl)isoindolin-1-one |
| a) | 2-(6-((2-(1H-indol-3-yl)ethyl)amino)- |
| | 4-(1-((4-methyl-4H-1,2,4-triazol-3- |
| | yl)methyl)cyclobutyl)pyridin-2-yl)-6- |
| | (((1-methylcyclobutyl)amino)methyl)- |
| | 4-(trifluoromethyl)isoindolin-1-one |
| b) | 2-(6-((2,2-difluoro-3-hydroxypropyl)amino)- |
| | 4-(1-((4-methyl-4H-1,2,4- |
| | triazol-3-yl)methyl)cyclobutyl)pyridin-2-yl)-6-(((1- |
| | methylcyclobutyl)amino)methyl)- |
| | 4-(trifluoromethyl)isoindolin-1-one |
| c) | 2-(6-((3-((dimethylamino)methyl)ben- |
| | zyl)amino)-4-(1-((4-methyl-4H- |
| | 1,2,4-triazol-3-yl)methyl)cyclo- |
| | butyl)pyridin-2-yl)-6-(((1- |
| | methylcyclobutyl)amino)methyl)-4- |
| | (trifluoromethyl)isoindolin-1-one |
| d) | 6-(((1-cyclopropylethyl)amino)methyl)- |
| | 2-(3-(3,3-difluoro-1-((4-methyl- |
| | 4H-1,2,4-triazol-3-yl)methyl)cyclobutyl)phenyl)-4- |
| | (trifluoromethyl)isoindolin-1-one formate |
| e) | 2-(6-(((1-methyl-1H-imidazol-4- |
| | yl)methyl)amino)-4-(1-((4-methyl-4H- |
| | 1,2,4-triazol-3-yl)methyl)cyclo- |
| | butyl)pyridin-2-yl)-6-(((1- |
| | methylcyclobutyl)amino)methyl)-4- |
| | (trifluoromethyl)isoindolin-1-one |
| f) | 2-(4-(1-((4-methyl-4H-1,2,4-triazol-3- |
| | yl)methyl)cyclobutyl)-6-((2-(pyridin- |
| | 4-yl)ethyl)amino)pyridin-2-yl)-6-(((1- |
| | methylcyclobutyl)amino)methyl)-4- |
| | (trifluoromethyl)isoindolin-1-one |
| g) | 2-(6-(3,3-dimethylbutyl)-4-(1- |
| | ((4-methyl-4H-1,2,4-triazol-3- |
| | yl)methyl)cyclobutyl)pyridin-2-yl)-6- |
| | (((1-methylcyclobutyl)amino)methyl)- |
| | 4-(trifluoromethyl)isoindolin-1-one |
| h) | 2-(4-(1-((4-methyl-4H-1,2,4-triazol- |
| | 3-yl)methyl)cyclobutyl)-6- |
| | (methylthio)pyridin-2-yl)-6-(((1-methyl- |
| | cyclobutyl)amino)methyl)-4- |
| | (trifluoromethyl)isoindolin-1-one |
| i) | 2-(4-(1-((4-methyl-4H-1,2,4-triazol- |
| | 3-yl)methyl)cyclobutyl)-6- |
| | phenylpyridin-2-yl)-6-(((1-methyl- |
| | cyclobutyl)amino)methyl)-4- |
| | (trifluoromethyl)isoindolin-1-one |
| j) | 2-(4-(1-((4-methyl-4H-1,2,4-triazol- |
| | 3-yl)methyl)cyclobutyl)-6- |
| | phenethylpyridin-2-yl)-6-(((1-methyl- |
| | cyclobutyl)amino)methyl)-4- |
| | (trifluoromethyl)isoindolin-1-one |
| a) | 2-(4-(1-((4-methyl-4H-1,2,4-triazol-3- |
| | yl)methyl)cyclobutyl)pyridin-2-yl)-6- |
| | (((1-methylcyclobutyl)amino)methyl)- |
| | 4-(trifluoromethyl)isoindolin-1-one |
| b) | 2-(6-(1-methyl-1H-pyrazol-4-yl)-4-(1- |
| | ((4-m ethyl-4 H-1,2,4-triazol-3- |
| | yl)methyl)cyclobutyl)pyridin-2-yl)-6- |
| | (((1-methylcyclobutyl)amino)methyl)- |
| | 4-(trifluoromethyl)isoindolin-1-one |
| c) | 2-(3-(1-(difluoro(4-methyl-4H-1,2,4-triazol-3- |
| | yl)methyl)cyclobutyl)phenyl)-6-(((1- |
| | methylcyclobutyl)amino)methyl)-4- |
| | (trifluoromethyl)isoindolin-1-one |
| d) | 2-(6-(((1-acetylpiperidin-4-yl)methyl)amino)- |
| | 4-(1-((4-methyl-4H-1,2,4-triazol- |
| | 3-yl)methyl)cyclobutyl)pyridin-2-yl)-6-(((1- |
| | methylcyclobutyl)amino)methyl)-4- |
| | (trifluoromethyl)isoindolin-1-one |
| | hydrochloride |
| e) | (S)-N-(6-(6-((2-isopropyl-4-methyl- |
| | piperazin-1-yl)methyl)-1-oxo-4- |
| | (trifluoromethyl)isoindolin-2-yl)-4-(1- |
| | ((4-methyl-4H-1,2,4-triazol-3- |
| | yl)methyl)cyclobutyl)pyridin-2-yl)acetamide |
| f) | 2-(3-(3-((4-methyl-4H-1,2,4-triazol-3- |
| | yl)methyl)oxetan-3-yl)phenyl)-6- |
| | (((1-methylcyclobutyl)amino)methyl)-4- |
| | (trifluoromethyl)isoindolin-1-one |
| g) | 2-(3-(3,3-difluoro-1-((4-methyl-4H-1,2,4-triazol-3- |
| | yl)methyl)cyclobutyl)phenyl)-6-(((3- |
| | methyloxetan-3-yl)amino)methyl)-4- |
| | (trifluoromethyl)isoindolin-1-one |
| h) | (S)-2-chloro-N-(6-(6-((2-isopropyl-4- |
| | methylpiperazin-1-yl)methyl)-1-oxo- |
| | 4-(trifluoromethyl)isoindolin-2-yl)- |
| | 4-(1-((4-methyl-4H-1,2,4-triazol-3- |
| | yl)methyl)cyclobutyl)pyridin-2-yl)acetamide |
| i) | 6-(((cyclopropylmethyl)amino)methyl)- |
| | 2-(3-(3,3-difluoro-1-((4-methyl- |
| | 4H-1,2,4-triazol-3-yl)methyl)cyclobutyl)phenyl)-4- |
| | (trifluoromethyl)isoindolin-1-one |
| j) | 2-(4-(1-((4-methyl-4H-1,2,4-triazol- |
| | 3-yl)methyl)cyclobutyl)-6-(((tetrahydro-2H- |
| | pyran-4-yl)methyl)amino)pyridin-2-yl)-6-(((1- |
| | methylcyclobutyl)amino)methyl)-4- |
| | (trifluoromethyl)isoindolin-1-one |
| a) | 2-(6-((2-(2-methoxyethoxy)ethyl)amino)- |
| | 4-(1-((4-methyl-4H-1,2,4- |
| | triazol-3-yl)methyl)cyclobutyl)pyridin-2-yl)-6-(((1- |
| | methylcyclobutyl)amino)methyl)-4- |
| | (trifluoromethyl)isoindolin-1-one |
| b) | 2-(6-((2-(dimethylamino)ethyl)amino)- |
| | 4-(1-((4-methyl-4H-1,2,4-triazol-3- |
| | yl)methyl)cyclobutyl)pyridin-2-yl)-6- |
| | (((1-methylcyclobutyl)amino)methyl)- |
| | 4-(trifluoromethyl)isoindolin-1-one |
| c) | 2-(4-(1-((4-methyl-4H-1,2,4-triazol- |
| | 3-yl)methyl)cyclobutyl)-6-((2-(2- |
| | oxopyrrolidin-1-yl)ethyl)amino)pyridin-2-yl)- |
| | 6-(((1-methylcyclobutyl)amino)methyl)-4- |
| | (trifluoromethyl)isoindolin-1-one |
| d) | 2-(4-(1-((4-methyl-4H-1,2,4-triazol-3- |
| | yl)methyl)cyclobutyl)-6-((piperidin- |
| | 4-ylmethyl)amino)pyridin-2-yl)-6-(((1- |
| | methylcyclobutyl)amino)methyl)-4- |
| | (trifluoromethyl)isoindolin-1-one |
| e) | 2-(4-(1-((4-methyl-4H-1,2,4-triazol- |
| | 3-yl)methyl)cyclobutyl)-6-((2- |
| | morpholinoethyl)amino)pyridin-2-yl)-6-(((1- |
| | methylcyclobutyl)amino)methyl)-4- |
| | (trifluoromethyl)isoindolin-1-one |
| f) | 2-(3-(3,3-difluoro-1-((4-methyl-4H-1,2,4-triazol-3- |
| | yl)methyl)cyclobutyl)phenyl)-6-(((3-fluoro-1- |
| | methylcyclobutyl)amino)methyl)-4- |
| | (trifluoromethyl)isoindolin-1-one |
| g) | 2-(6-isobutyl-4-(1-((4-methyl-4H-1,2,4-triazol-3- |
| | yl)methyl)cyclobutyl)pyridin-2-yl)-6- |
| | (((1-methylcyclobutyl)amino)methyl)- |
| | 4-(trifluoromethyl)isoindolin-1-one formate |
| h) | 2-(4-(1-((4-methyl-4H-1,2,4- |
| | triazol-3-yl)methyl)cyclobutyl)-6- |
| | propylpyridin-2-yl)-6-(((1- |
| | methylcyclobutyl)amino)methyl)-4- |
| | (trifluoromethyl)isoindolin-1-one |
| i) | 2-(3-(3,3-difluoro-1-((4-methyl-4H-1,2,4- |
| | triazol-3-yl)methyl)cyclobutyl)phenyl)-6-(((1- |
| | methylcyclopentyl)amino)methyl)-4- |
| | (trifluoromethyl)isoindolin-1-one formate |
| j) | 2-(6-benzyl-4-(1-((4-methyl-4H-1,2,4-triazol-3- |
| | yl)methyl)cyclobutyl)pyridin-2-yl)-6- |
| | (((1-methylcyclobutyl)amino)methyl)- |
| | 4-(trifluoromethyl)isoindolin-1-one |
| a) | 2-(6-butyl-4-(1-((4-methyl-4H-1,2,4-triazol-3- |
| | yl)methyl)cyclobutyl)pyridin-2-yl)-6- |
| | (((1-methylcyclobutyl)amino)methyl)- |
| | 4-(trifluoromethyl)isoindolin-1-one |
| b) | 2-(3-(3-(difluoro(4-methyl-4H-1,2,4- |
| | triazol-3-yl)methyl)oxetan-3-yl)phenyl)- |
| | 6-(((1-methylcyclobutyl)amino)methyl)-4- |
| | (trifluoromethyl)isoindolin-1-one |
| c) | 2-(6-(isobutylamino)-4-(1- |
| | ((4-methyl-4H-1,2,4-triazol-3- |
| | yl)methyl)cyclobutyl)pyridin-2-yl)-6- |
| | (((1-methylcyclobutyl)amino)methyl)- |
| | 4-(trifluoromethyl)isoindolin-1-one |
| d) | 2-(6-(ethylthio)-4-(1-((4-methyl-4H-1,2,4- |
| | triazol-3-yl)methyl)cyclobutyl)pyridin-2-yl)-6- |
| | (((1-methylcyclobutyl)amino)methyl)- |
| | 4-(trifluoromethyl)isoindolin-1-one |
| e) | 2-(4-(1-((4-methyl-4H-1,2,4-triazol- |
| | 3-yl)methyl)cyclobutyl)-6- |
| | (naphthalen-2-yl)pyridin-2-yl)-6-(((1- |
| | methylcyclobutyl)amino)methyl)-4- |
| | (trifluoromethyl)isoindolin-1-one |
| f) | 2-(6-chloro-4-(1-((4-methyl-4H-1,2,4-triazol-3- |
| | yl)methyl)cyclobutyl)pyridin-2-yl)-6-(((1- |
| | methylcyclobutyl)amino)methyl)- |
| | 4-(trifluoromethyl)isoindolin-1-one formate |
| g) | 2-(3-(3,3-difluoro-1-((4-methyl-4H-1,2,4-triazol-3- |
| | yl)methyl)cyclobutyl)phenyl)-6-((((1- |
| | methylcyclopropyl)methyl)amino)methyl)- |
| | 4-(trifluoromethyl)isoindolin-1- |
| | one formate |
| h) | 2-(3-(3,3-difluoro-1-((4-methyl-4H-1,2,4-triazol-3- |
| | yl)methyl)cyclobutyl)phenyl)-6-(((1,3- |
| | dimethylcyclobutyl)amino)methyl)- |
| | 4-(trifluoromethyl)isoindolin-1-one formate |
| i) | 2-(3-(3,3-difluoro-1-((4-methyl-4H-1,2,4-triazol-3- |
| | yl)methyl)cyclobutyl)phenyl)-6- |
| | (((3-methylbicyclo[1.1.1]pentan-1- |
| | yl)amino)methyl)-4-(trifluoromethyl)isoindolin |
| | 1-one formate |
| a) | 2-(3-(3,3-difluoro-1-((4- |
| | methyl-4H-1,2,4-triazol-3- |
| | yl)methyl)cyclobutyl)phenyl)-4- |
| | (trifluoromethyl)-6-(((1,2,2- |
| | trimethylcyclobutyl)amino)methyl)isoindolin- |
| | 1-one formate |
| b) | 6-((bicyclo[1.1.1]pentan-1- |
| | ylamino)methyl)-2-(3-(3,3-difluoro-1-((4-methyl- |
| | 4H-1,2,4-triazol-3-yl)methyl)cyclobutyl)phenyl)-4- |
| | (trifluoromethyl)isoindolin-1-one |
| c) | 2-(3-(3,3-difluoro-1-((4-methyl-4H-1,2,4-triazol-3- |
| | yl)methyl)cyclobutyl)phenyl)-6-((((2,2- |
| | dimethyl-1,3-dioxolan-4- |
| | yl)methyl)amino)methyl)-4- |
| | (trifluoromethyl)isoindolin-1-one formate |
| d) | 2-(3-(3,3-difluoro-1-((4-methyl-4H-1,2,4- |
| | triazol-3-yl)methyl)cyclobutyl)phenyl)- |
| | 6-((((tetrahydrofuran-3- |
| | yl)methyl)amino)methyl)-4- |
| | (trifluoromethyl)isoindolin-1-one formate |
| e) | 2-(6-ethoxy-4-(1-((4-methyl-4H-1,2,4-triazol-3- |
| | yl)methyl)cyclobutyl)pyridin-2-yl)-6- |
| | (((1-methylcyclobutyl)amino)methyl)- |
| | 4-(trifluoromethyl)isoindolin-1-one |
| f) | 2-(6-(benzyloxy)-4-(1-((4-methyl-4H-1,2,4- |
| | triazol-3-yl)methyl)cyclobutyl)pyridin-2-yl)-6- |
| | (((1-methylcyclobutyl)amino)methyl)- |
| | 4-(trifluoromethyl)isoindolin-1-one |
| g) | 2-(6-ethyl-4-(1-((4-methyl-4H-1,2,4-triazol-3- |
| | yl)methyl)cyclobutyl)pyridin-2-yl)-6- |
| | (((1-methylcyclobutyl)amino)methyl)- |
| | 4-(trifluoromethyl)isoindolin-1-one |
| h) | 2-(6-cyclopropyl-4-(1-((4-methyl-4H-1,2,4- |
| | triazol-3-yl)methyl)cyclobutyl)pyridin-2-yl)-6- |
| | (((1-methylcyclobutyl)amino)methyl)- |
| | 4-(trifluoromethyl)isoindolin-1-one |
| i) | 2-(3-(3,3-difluoro-1-((4-methyl-4H-1,2,4- |
| | triazol-3-yl)methyl)cyclobutyl)phenyl)- |
| | 6-((neopentylamino)methyl)-4- |
| | (trifluoromethyl)isoindolin-1-one |
| j) | 6-((cyclopentylamino)methyl)-2-(3- |
| | (3,3-difluoro-1-((4-methyl-4H-1,2,4- |
| | triazol-3-yl)methyl)cyclobutyl)phenyl)- |
| | 4-(trifluoromethyl)isoindolin-1-one |
| a) | (S)-6-((2-isopropyl-4-methylpiperazin- |
| | 1-yl)methyl)-2-(6-methoxy-4-(1- |
| | ((4-methyl-4H-1,2,4-triazol-3- |
| | yl)methyl)cyclobutyl)pyridin-2-yl)-4- |
| | (trifluoromethyl)isoindolin-1-one |
| b) | 6-((2-oxaspiro[3.3]heptan-6- |
| | ylamino)methyl)-2-(3-(3,3-difluoro-1-((4-methyl- |
| | 4H-1,2,4-triazol-3-yl)methyl)cyclobutyl)phenyl)-4- |
| | (trifluoromethyl)isoindolin-1-one |
| c) | 2-(3-(3,3-difluoro-1-((4-methyl-4H-1,2,4-triazol-3- |
| | yl)methyl)cyclobutyl)phenyl)-6-(((1- |
| | (methoxymethyl)cyclopropyl)amino)methyl)-4- |
| | (trifluoromethyl)isoindolin-1-one |
| d) | 2-(3-(3,3-difluoro-1-((4-methyl-4H-1,2,4-triazol-3- |
| | yl)methyl)cyclobutyl)phenyl)-6- |
| | (((oxetan-3-ylmethyl)amino)methyl)-4- |
| | (trifluoromethyl)isoindolin-1-one |
| e) | 2-(3-(3,3-difluoro-1-((4- |
| | methyl-4H-1,2,4-triazol-3- |
| | yl)methyl)cyclobutyl)phenyl)-6-(((1- |
| | ethylcyclobutyl)amino)methyl)-4- |
| | (trifluoromethyl)isoindolin-1-one formate |
| f) | (R)-6-((sec-butylamino)methyl)-2-(3- |
| | (3,3-difluoro-1-((4-methyl-4H-1,2,4- |
| | triazol-3-yl)methyl)cyclobutyl)phenyl)- |
| | 4-(trifluoromethyl)isoindolin-1-one |
| g) | (S)-6-((sec-butylamino)methyl)-2-(3- |
| | (3,3-difluoro-1-((4-methyl-4H-1,2,4- |
| | triazol-3-yl)methyl)cyclobutyl)phenyl)- |
| | 4-(trifluoromethyl)isoindolin-1-one |
| h) | 2-(3-(3,3-difluoro-1-((4-methyl-4H-1,2,4-triazol-3- |
| | yl)methyl)cyclobutyl)phenyl)-6- |
| | (((1-fluoro-2-methylpropan-2-yl)amino)methyl)- |
| | 4-(trifluoromethyl)isoindolin-1-one |
| i) | 2-(3-(3,3-difluoro-1-((4-methyl-4H-1,2,4-triazol-3- |
| | yl)methyl)cyclobutyl)phenyl)-6-(((2-methoxy-2- |
| | methylpropyl)amino)methyl)-4- |
| | (trifluoromethyl)isoindolin-1-one |
| j) | 2-(3-(3,3-difluoro-1-((4-methyl-4H-1,2,4-triazol-3- |
| | yl)methyl)cyclobutyl)phenyl)-6-((4- |
| | methyl-2-(trifluoromethyl)piperazin-1- |
| | yl)methyl)-4-(trifluoromethyl)isoindolin-1-one |
| a) | (R)-6-((4,4-difluoro-3-methylpiperidin- |
| | 1-yl)methyl)-2-(6-ethoxy-4-(3-((4-methyl-4H-1,2,4- |
| | triazol-3-yl)methyl)oxetan-3-yl)pyridin-2-yl)-4- |
| | (trifluoromethyl)isoindolin-1-one |
| b) | 2-(3-(3-((S)-fluoro(4-(trifluoromethyl)- |
| | 1H-pyrazol-3-yl)methyl)oxetan-3- |
| | yl)phenyl)-6-(((S)-2-isopropyl- |
| | 4-methylpiperazin-1-yl)methyl)-4- |
| | (trifluoromethyl)isoindolin-1-one |
| c) | 2-(3-(3-((R)-fluoro(4-(trifluoromethyl)- |
| | 1H-pyrazol-3-yl)methyl)oxetan-3- |
| | yl)phenyl)-6-(((S)-2-isopropyl-4- |
| | methylpiperazin-1-yl)methyl)-4- |
| | (trifluoromethyl)isoindolin-1-one |
| d) | 2-(6-((cyclopropylmethyl)amino)- |
| | 4-(1-((4-methyl-4H-1,2,4-triazol-3- |
| | yl)methyl)cyclobutyl)pyridin-2-yl)-6- |
| | (((1-methylcyclobutyl)amino)methyl)- |
| | 4-(trifluoromethyl)isoindolin-1-one |
| e) | (S)-N-(6-(6-((2-isopropyl-4- |
| | methylpiperazin-1-yl)methyl)-1-oxo-4- |
| | (trifluoromethyl)isoindolin-2-yl)-4- |
| | (1-((4-methyl-4H-1,2,4-triazol-3- |
| | yl)methyl)cyclobutyl)pyridin-2-yl)acrylamide |
| f) | 2-(4-(1-((4-methyl-4H-1,2,4-triazol-3- |
| | yl)methyl)cyclobutyl)-6-((pyridin-4- |
| | ylmethyl)amino)pyridin-2-yl)-6-(((1- |
| | methylcyclobutyl)amino)methyl)-4- |
| | (trifluoromethyl)isoindolin-1-one hydrochloride |
| g) | 2-(6-(cyclopentyloxy)-4-(3- |
| | ((4-methyl-4H-1,2,4-triazol-3- |
| | yl)methyl)oxetan-3-yl)pyridin-2-yl)-4- |
| | (trifluoromethyl)isoindolin-1-one |
| h) | 2-(6-(bicyclo[2.1.1]hexan-5-ylamino)- |
| | 4-(3-((4-methyl-4H-1,2,4-triazol-3- |
| | yl)methyl)oxetan-3-yl)pyridin-2-yl)- |
| | 4-(trifluoromethyl)isoindolin-1-one |
| i) | 2-(3-(1-(difluoro(4-methyl-4H- |
| | 1,2,4-triazol-3-yl)methyl)-3,3- |
| | difluorocyclobutyl)phenyl)-6-((3-hydroxy- |
| | 3-methylazetidin-1-yl)methyl)- |
| | 4-(trifluoromethyl)isoindolin-1-one |
| j) | (S)-6-(1-aminoethyl)-2-(3-(3,3-difluoro-1- |
| | ((4-methyl-4H-1,2,4-triazol-3- |
| | yl)methyl)cyclobutyl)phenyl)-4- |
| | (trifluoromethyl)isoindolin-1-one |
| k) | (R)-6-(1-aminoethyl)-2-(3-(3,3-difluoro- |
| | 1-((4-methyl-4H-1,2,4-triazol-3- |
| | yl)methyl)cyclobutyl)phenyl)-4- |
| | (trifluoromethyl)isoindolin-1-one |
| l) | (R)-6-(aminomethyl)-2-(3-(1- |
| | (fluoro(4-methyl-4H-1,2,4-triazol-3- |
| | yl)methyl)cyclobutyl)phenyl)-4- |
| | (trifluoromethyl)isoindolin-1-one |
| a) | (S)-6-(aminomethyl)-2-(3-(1- |
| | (fluoro(4-methyl-4H-1,2,4-triazol-3- |
| | yl)methyl)cyclobutyl)phenyl)-4- |
| | (trifluoromethyl)isoindolin-1-one |
| b) | 2-(3-(3,3-difluoro-1-((R)-fluoro(4- |
| | methyl-4H-1,2,4-triazol-3- |
| | yl)methyl)cyclobutyl)phenyl)-6-(1-((S)- |
| | 2-isopropyl-4-methylpiperazin-1- |
| | yl)ethyl)-4-(trifluoromethyl)isoindolin-1-one |
| c) | 6-((S)-1-amino-2-cyclopropylethyl)- |
| | 2-(3-(3-((R)-fluoro(4-methyl-4H- |
| | 1,2,4-triazol-3-yl)methyl)oxetan-3-yl)phenyl)-4- |
| | (trifluoromethyl)isoindolin-1-one |
| d) | 6-((R)-1-amino-2-cyclopropylethyl)- |
| | 2-(3-(3-((R)-fluoro(4-methyl-4H- |
| | 1,2,4-triazol-3-yl)methyl)oxetan-3-yl)phenyl)-4- |
| | (trifluoromethyl)isoindolin-1-one |
| e) | 6-((S)-amino(cyclobutyl)methyl)-2-(3-(3- |
| | ((R)-fluoro(4-methyl-4H-1,2,4- |
| | triazol-3-yl)methyl)oxetan-3-yl)phenyl)- |
| | 4-(trifluoromethyl)isoindolin-1- |
| | one |
| f) | 6-((R)-amino(cyclobutyl)methyl)-2-(3- |
| | (3-((R)-fluoro(4-methyl-4H-1,2,4- |
| | triazol-3-yl)methyl)oxetan-3-yl)phenyl)- |
| | 4-(trifluoromethyl)isoindolin-1- |
| | one |
| g) | (R)-6-(aminomethyl)-2-(3-(3,3-difluoro- |
| | 1-(fluoro(4-methyl-4H-1,2,4- |
| | triazol-3-yl)methyl)cyclobutyl)phenyl)- |
| | 4-(trifluoromethyl)isoindolin-1-one |
| | formate |
| h) | (S)-6-(aminomethyl)-2-(3-(3,3-difluoro- |
| | 1-(fluoro(4-methyl-4H-1,2,4- |
| | triazol-3-yl)methyl)cyclobutyl)phenyl)- |
| | 4-(trifluoromethyl)isoindolin-1-one |
| | formate |
| i) | (R)-2-(3-(3,3-difluoro-1-(fluoro(4- |
| | methyl-4H-1,2,4-triazol-3- |
| | yl)methyl)cyclobutyl)phenyl)- |
| | 6-(pyrrolidin-1-ylmethyl)-4- |
| | (trifluoromethyl)isoindolin-1-one |
| j) | (S)-2-(3-(3,3-difluoro-1-(fluoro(4- |
| | methyl-4H-1,2,4-triazol-3- |
| | yl)methyl)cyclobutyl)phenyl)- |
| | 6-(pyrrolidin-1-ylmethyl)-4- |
| | (trifluoromethyl)isoindolin-1-one |
| a) | (R)-6-(azetidin-1-ylmethyl)-2-(3- |
| | (3,3-difluoro-1-(fluoro(4-methyl-4H- |
| | 1,2,4-triazol-3-yl)methyl)cyclobutyl)phenyl)- |
| | 4-(trifluoromethyl)isoindolin- |
| | 1-one |
| b) | (S)-6-(azetidin-1-ylmethyl)-2-(3- |
| | (3,3-difluoro-1-(fluoro(4-methyl-4H- |
| | 1,2,4-triazol-3-yl)methyl)cyclobutyl)phenyl)- |
| | 4-(trifluoromethyl)isoindolin- |
| | 1-one |
| c) | 6-(aminomethyl)-2-(3-(1-(difluoro(4-methyl- |
| | 4H-1,2,4-triazol-3-yl)methyl)- |
| | 3,3-difluorocyclobutyl)phenyl)-4- |
| | (trifluoromethyl)isoindolin-1-one |
| d) | 6-(aminomethyl)-2-(3-(3,3-difluoro- |
| | 1-((4-methyl-4H-1,2,4-triazol-3- |
| | yl)methyl)cyclobutyl)phenyl)-4- |
| | (trifluoromethyl)isoindolin-1-one |
| e) | (S)-2-(3-(3,3-difluoro-1- |
| | ((4-methyl-4H-1,2,4-triazol-3- |
| | yl)methyl)cyclobutyl)phenyl)-6-((2-ethyl- |
| | 4-methylpiperazin-1-yl)methyl)- |
| | 4-(trifluoromethyl)isoindolin-1-one |
| f) | (S)-6-((2-cyclopropyl-4-methylpiperazin- |
| | 1-yl)methyl)-2-(3-(3,3-difluoro- |
| | 1-((4-methyl-4H-1,2,4-triazol-3- |
| | yl)methyl)cyclobutyl)phenyl)-4- |
| | (trifluoromethyl)isoindolin-1-one |
| g) | 6-((3-cyclopropyl-4-methylpiperazin-1- |
| | yl)methyl)-2-(3-(3,3-difluoro-1- |
| | ((4-methyl-4H-1,2,4-triazol-3- |
| | yl)methyl)cyclobutyl)phenyl)-4- |
| | (trifluoromethyl)isoindolin-1-one |
| h) | (S)-2-(6-ethoxy-4-(1-((4-methyl-4H-1,2,4- |
| | triazol-3-yl)methyl)cyclobutyl)pyridin-2-yl)-6- |
| | ((2-isopropyl-4-methylpiperazin-1- |
| | yl)methyl)-4-(trifluoromethyl)isoindolin-1-one |
| i) | 6-(((S)-4-cyclopropyl-2-isopro- |
| | pylpiperazin-1-yl)methyl)-2-(3-(3-((R)- |
| | fluoro(4-methyl-4H-1,2,4-triazol-3- |
| | yl)methyl)oxetan-3-yl)phenyl)-4- |
| | (trifluoromethyl)isoindolin-1-one |
| j) | 2-(3-(3-((R)-fluoro(4-methyl-4H- |
| | 1,2,4-triazol-3-yl)methyl)oxetan-3- |
| | yl)phenyl)-6-(((R)-3-isopropyl-4- |
| | methylpiperazin-1-yl)methyl)-4- |
| | (trifluoromethyl)isoindolin-1-one |
| a) | (S)-6-((2-isopropyl-4-methylpiperazin- |
| | 1-yl)methyl)-2-(3-(3-((4-methyl- |
| | 4H-1,2,4-triazol-3-yl)methyl)oxetan-3-yl)phenyl)- |
| | 4-(trifluoromethyl)isoindolin-1-one |
| b) | (S)-6-((2-isopropyl-4-methylpiperazin-1-yl)methyl)- |
| | 2-(3-(isopropylamino)-5-(1-((4-methyl-4H- |
| | 1,2,4-triazol-3-yl)methyl)cyclobutyl)phenyl)-4- |
| | (trifluoromethyl)isoindolin-1-one |
| c) | (S)-6-((2-isopropyl-4-methylpiperazin-1-yl)methyl)- |
| | 2-(6-(isopropylamino)-4-(1-((4-methyl-4H- |
| | 1,2,4-triazol-3-yl)methyl)cyclobutyl)pyridin-2-yl)- |
| | 4-(trifluoromethyl)isoindolin-1-one |
| d) | (S)-6-((2-isopropyl-4-methylpiperazin- |
| | 1-yl)methyl)-2-(3-(1-((4-methyl- |
| | 4H-1,2,4-triazol-3-yl)methyl)cyclobutyl)phenyl)-4- |
| | (trifluoromethyl)isoindolin-1-one |
| e) | 2-(3-(3,3-difluoro-1-((4-methyl-4H-1,2,4-triazol-3- |
| | yl)methyl)cyclobutyl)phenyl)-6-((4- |
| | methylpiperazin-1-yl)methyl)-4- |
| | (trifluoromethyl)isoindolin-1-one |
| f) | 2-(3-(3,3-difluoro-1-((4-methyl-4H-1,2,4-triazol-3- |
| | yl)methyl)cyclobutyl)phenyl)-6-((3-methyl-3,6- |
| | diazabicyclo[3.1.1]heptan-6-yl)methyl)- |
| | 4-(trifluoromethyl)isoindolin-1- |
| | one |
| g) | 2-(3-(3,3-difluoro-1-((4-methyl-4H-1,2,4-triazol-3- |
| | yl)methyl)cyclobutyl)phenyl)-6-((3-methyl-3,6- |
| | diazabicyclo[3.1.1]heptan-6-yl)methyl)- |
| | 4-(trifluoromethyl)isoindolin-1- |
| | one formate |
| h) | 2-(3-(3,3-difluoro-1-((4-methyl-4H-1,2,4-triazol-3- |
| | yl)methyl)cyclobutyl)phenyl)-6-(((1S,4S)-5- |
| | methyl-2,5-diazabicyclo[2.2.1]heptan-2-yl)methyl)- |
| | 4-(trifluoromethyl)isoindolin-1- |
| | one |
| i) | 2-(3-(3,3-difluoro-1-((4-methyl-4H-1,2,4-triazol-3- |
| | yl)methyl)cyclobutyl)phenyl)- |
| | 6-((isopropylamino)methyl)-4- |
| | (trifluoromethyl)isoindolin-1-one |
| a) | 2-(3-(3,3-difluoro-1-((4-methyl-4H-1,2,4-triazol-3- |
| | yl)methyl)cyclobutyl)phenyl)-6- |
| | ((isopropyl(methyl)amino)methyl)-4- |
| | (trifluoromethyl)isoindolin-1-one |
| b) | (R)-2-(3-(3,3-difluoro-1-(fluoro(4- |
| | methyl-4H-1,2,4-triazol-3- |
| | yl)methyl)cyclobutyl)phenyl)-6- |
| | ((isopropylamino)methyl)-4- |
| | (trifluoromethyl)isoindolin-1-one |
| c) | (S)-2-(3-(3,3-difluoro-1-(fluoro(4- |
| | methyl-4H-1,2,4-triazol-3- |
| | yl)methyl)cyclobutyl)phenyl)- |
| | 6-((isopropylamino)methyl)-4- |
| | (trifluoromethyl)isoindolin-1-one |
| d) | (R)-2-(3-(3,3-difluoro-1-(fluoro(4- |
| | methyl-4H-1,2,4-triazol-3- |
| | yl)methyl)cyclobutyl)phenyl)-6- |
| | ((isopropyl(methyl)amino)methyl)-4- |
| | (trifluoromethyl)isoindolin-1-one |
| e) | (S)-2-(3-(3,3-difluoro-1-(fluoro(4- |
| | methyl-4H-1,2,4-triazol-3- |
| | yl)methyl)cyclobutyl)phenyl)-6- |
| | ((isopropyl(methyl)amino)methyl)-4- |
| | (trifluoromethyl)isoindolin-1-one |
| f) | (S)-2-(3-(3,3-difluoro-1-((4-methyl-4H-1,2,4- |
| | triazol-3-yl)methyl)cyclobutyl)phenyl)-6-((3-ethyl- |
| | 4-methylpiperazin-1-yl)methyl)- |
| | 4-(trifluoromethyl)isoindolin-1-one |
| g) | 6-((1H-imidazol-4-yl)methoxy)-2-(3-(3,3- |
| | difluoro-1-((4-methyl-4H-1,2,4- |
| | triazol-3-yl)methyl)cyclobutyl)phenyl)- |
| | 4-(trifluoromethyl)isoindolin-1-one |
| h) | (S)-6-cyclopropyl-N-(3-(3,3-difluoro-1- |
| | (fluoro(4-methyl-4H-1,2,4-triazol- |
| | 3-yl)methyl)cyclobutyl)phenyl)-4-((3- |
| | (hydroxymethyl)-3-methylazetidin- |
| | 1-yl)methyl)picolinamide |
| i) | (R)-2-(3-(3,3-difluoro-1-((4-methyl-4H-1,2,4- |
| | triazol-3-yl)methyl)cyclobutyl)phenyl)-6- |
| | ((3-methylpiperazin-1-yl)methyl)-4- |
| | (trifluoromethyl)isoindolin-1-one |
| j) | (S)-2-(3-(3,3-difluoro-1-((4-methyl-4H-1,2,4- |
| | triazol-3-yl)methyl)cyclobutyl)phenyl)-6- |
| | ((3-methylpiperazin-1-yl)methyl)-4- |
| | (trifluoromethyl)isoindolin-1-one |
| a) | (R)-2-(3-(3,3-difluoro-1-((4-methyl-4H-1,2,4- |
| | triazol-3-yl)methyl)cyclobutyl)phenyl)-6- |
| | ((3-ethylpiperazin-1-yl)methyl)-4- |
| | (trifluoromethyl)isoindolin-1-one |
| b) | (S)-2-(3-(3,3-difluoro-1-((4-methyl-4H-1,2,4- |
| | triazol-3-yl)methyl)cyclobutyl)phenyl)-6-((3- |
| | ethylpiperazin-1-yl)methyl)-4- |
| | (trifluoromethyl)isoindolin-1-one |
| c) | (R)-2-(3-(3,3-difluoro-1-((4-methyl-4H-1,2,4- |
| | triazol-3-yl)methyl)cyclobutyl)phenyl)-6-((3- |
| | isopropylpiperazin-1-yl)methyl)-4- |
| | (trifluoromethyl)isoindolin-1-one |
| d) | (S)-2-(3-(3,3-difluoro-1-((4-methyl-4H-1,2,4- |
| | triazol-3-yl)methyl)cyclobutyl)phenyl)-6-((3- |
| | isopropylpiperazin-1-yl)methyl)-4- |
| | (trifluoromethyl)isoindolin-1-one |
| e) | (R)-6-((3-cyclopropylpiperazin-1- |
| | yl)methyl)-2-(3-(3,3-difluoro-1-((4-methyl-4H- |
| | 1,2,4-triazol-3-yl)methyl)cyclobutyl)phenyl)-4- |
| | (trifluoromethyl)isoindolin-1-one |
| f) | (S)-6-((3-cyclopropylpiperazin-1- |
| | yl)methyl)-2-(3-(3,3-difluoro-1-((4-methyl-4H- |
| | 1,2,4-triazol-3-yl)methyl)cyclobutyl)phenyl)-4- |
| | (trifluoromethyl)isoindolin-1-one |
| g) | (S)-2-(3-(1-(difluoro(4-methyl-4H- |
| | 1,2,4-triazol-3-yl)methyl)-3,3- |
| | difluorocyclobutyl)phenyl)-6-((2- |
| | isopropyl-4-methylpiperazin-1- |
| | yl)methyl)-4-(trifluoromethyl)isoindolin-1-one |
| h) | (R)-6-((2-cyclopropyl-4-methylpiperazin- |
| | 1-yl)methyl)-2-(3-(3,3-difluoro- |
| | 1-((4-methyl-4H-1,2,4-triazol-3- |
| | yl)methyl)cyclobutyl)phenyl)-4- |
| | (trifluoromethyl)isoindolin-1-one |
| i) | (S)-2-(3-(3,3-difluoro-1-((4-methyl-4H-1,2,4- |
| | triazol-3-yl)methyl)cyclobutyl)phenyl)-6-((2-ethyl- |
| | 4-methylpiperazin-1-yl)methyl)- |
| | 4-(trifluoromethyl)isoindolin-1-one |
| j) | (R)-6-(amino(cyclopropyl)methyl)-2-(3- |
| | (3,3-difluoro-1-((4-methyl-4H- |
| | 1,2,4-triazol-3-yl)methyl)cyclobutyl)phenyl)- |
| | 4-(trifluoromethyl)isoindolin- |
| | 1-one |
| a) | (R)-6-((2-cyclopropylpiperazin-1- |
| | yl)methyl)-2-(3-(3,3-difluoro-1-((4-methyl-4H- |
| | 1,2,4-triazol-3-yl)methyl)cyclobutyl)phenyl)-4- |
| | (trifluoromethyl)isoindolin-1-one |
| b) | 2-(3-(3,3-difluoro-1-((4-methyl-4H-1,2,4-triazol-3- |
| | yl)methyl)cyclobutyl)phenyl)-6-(piperazin-1- |
| | ylmethyl)-4-(trifluoromethyl)isoindolin-1-one |
| c) | (R)-2-(3-(3,3-difluoro-1-((4-methyl-4H-1,2,4- |
| | triazol-3-yl)methyl)cyclobutyl)phenyl)-6-((3- |
| | isopropyl-4-methylpiperazin-1- |
| | yl)methyl)-4-(trifluoromethyl)isoindolin-1-one |
| d) | (S)-2-(3-(3,3-difluoro-1-((4-methyl-4H-1,2,4- |
| | triazol-3-yl)methyl)cyclobutyl)phenyl)-6-((3- |
| | isopropyl-4-methylpiperazin-1- |
| | yl)methyl)-4-(trifluoromethyl)isoindolin-1-one |
| e) | (S)-2-(3-(3,3-difluoro-1-((4-methyl-4H-1,2,4- |
| | triazol-3-yl)methyl)cyclobutyl)phenyl)-6-((3- |
| | hydroxy-3-methylpiperidin-1- |
| | yl)methyl)-4-(trifluoromethyl)isoindolin-1-one |
| f) | (R)-2-(3-(3,3-difluoro-1-((4-methyl-4H-1,2,4- |
| | triazol-3-yl)methyl)cyclobutyl)phenyl)-6- |
| | ((3-hydroxy-3-methylpiperidin-1- |
| | yl)methyl)-4-(trifluoromethyl)isoindolin-1-one |
| g) | 2-(3-(3,3-difluoro-1-((4-methyl-4H-1,2,4- |
| | triazol-3-yl)methyl)cyclobutyl)phenyl)-6-((1- |
| | methylazetidin-3-yl)methyl)-4- |
| | (trifluoromethyl)isoindolin-1-one formate |
| h) | (S)-2-(3-(3,3-difluoro-1-((4-methyl-4H-1,2,4- |
| | triazol-3-yl)methyl)cyclobutyl)phenyl)-6-((3- |
| | methoxypiperidin-1-yl)methyl)-4- |
| | (trifluoromethyl)isoindolin-1-one |
| i) | 2-(3-(3,3-difluoro-1-((4-methyl-4H-1,2,4-triazol- |
| | 3-yl)methyl)cyclobutyl)phenyl)-6-((4-methyl- |
| | 2-oxopiperazin-1-yl)methyl)-4- |
| | (trifluoromethyl)isoindolin-1-one |
| j) | (R)-2-(3-(3,3-difluoro-1-(fluoro(4- |
| | methyl-4H-1,2,4-triazol-3- |
| | yl)methyl)cyclobutyl)phenyl)- |
| | 6-((propylamino)methyl)-4- |
| | (trifluoromethyl)isoindolin-1-one |
| a) | (S)-2-(3-(3,3-difluoro-1-(fluoro(4- |
| | methyl-4H-1,2,4-triazol-3- |
| | yl)methyl)cyclobutyl)phenyl)- |
| | 6-((propylamino)methyl)-4- |
| | (trifluoromethyl)isoindolin-1-one |
| b) | (R)-2-(3-(3,3-difluoro-1-(fluoro(4- |
| | methyl-4H-1,2,4-triazol-3- |
| | yl)methyl)cyclobutyl)phenyl)-6-(((3- |
| | fluoropropyl)amino)methyl)-4- |
| | (trifluoromethyl)isoindolin-1-one |
| c) | (S)-2-(3-(3,3-difluoro-1-(fluoro(4- |
| | methyl-4H-1,2,4-triazol-3- |
| | yl)methyl)cyclobutyl)phenyl)-6-(((3- |
| | fluoropropyl)amino)methyl)-4- |
| | (trifluoromethyl)isoindolin-1-one |
| d) | (R)-6-(((cyclopropylmethyl)amino)methyl)- |
| | 2-(3-(3,3-difluoro-1-(fluoro(4- |
| | methyl-4H-1,2,4-triazol-3- |
| | yl)methyl)cyclobutyl)phenyl)-4- |
| | (trifluoromethyl)isoindolin-1-one |
| e) | (S)-6-(((cyclopropylmethyl)amino)methyl)- |
| | 2-(3-(3,3-difluoro-1-(fluoro(4- |
| | methyl-4H-1,2,4-triazol-3- |
| | yl)methyl)cyclobutyl)phenyl)-4- |
| | (trifluoromethyl)isoindolin-1-one |
| f) | (R)-2-(3-(3,3-difluoro-1-(fluoro(4- |
| | methyl-4H-1,2,4-triazol-3- |
| | yl)methyl)cyclobutyl)phenyl)-6- |
| | ((isobutylamino)methyl)-4- |
| | (trifluoromethyl)isoindolin-1-one |
| g) | (S)-2-(3-(3,3-difluoro-1-(fluoro(4- |
| | methyl-4H-1,2,4-triazol-3- |
| | yl)methyl)cyclobutyl)phenyl)-6- |
| | ((isobutylamino)methyl)-4- |
| | (trifluoromethyl)isoindolin-1-one |
| h) | (S)-6-(amino(cyclopropyl)methyl)-2- |
| | (3-(3,3-difluoro-1-((4-methyl-4H- |
| | 1,2,4-triazol-3-yl)methyl)cyclobutyl)phenyl)- |
| | 4-(trifluoromethyl)isoindolin- |
| | 1-one |
| i) | 2-(3-(3-((R)-(4-cyclohexyl-4H-1,2,4- |
| | triazol-3-yl)fluoromethyl)oxetan-3- |
| | yl)phenyl)-6-(((S)-2-isopropyl-4- |
| | methylpiperazin-1-yl)methyl)-4- |
| | (trifluoromethyl)isoindolin-1-one |
| j) | 2-(3-(3-((S)-(4-cyclohexyl-4H-1,2,4- |
| | triazol-3-yl)fluoromethyl)oxetan-3- |
| | yl)phenyl)-6-(((S)-2-isopropyl-4- |
| | methylpiperazin-1-yl)methyl)-4- |
| | (trifluoromethyl)isoindolin-1-one |
| a) | 2-(3-(isopropylamino)-5-(1-((4- |
| | methyl-4H-1,2,4-triazol-3- |
| | yl)methyl)cyclobutyl)phenyl)- |
| | 6-((propylamino)methyl)-4- |
| | (trifluoromethyl)isoindolin-1-one |
| b) | 6-(((cyclopropylmethyl)amino)methyl)- |
| | 2-(3-(ethylamino)-5-(1-((4-methyl- |
| | 4H-1,2,4-triazol-3-yl)methyl)cyclobutyl)phenyl)-4- |
| | (trifluoromethyl)isoindolin-1-one |
| c) | 2-(3-(ethylamino)-5-(1-((4-methyl-4H-1,2,4- |
| | triazol-3-yl)methyl)cyclobutyl)phenyl)- |
| | 6-((propylamino)methyl)- |
| | 4-(trifluoromethyl)isoindolin-1-one |
| d) | 6-(((cyclopropylmethyl)amino)methyl)- |
| | 2-(3-(isopropylamino)-5-(1-((4-methyl- |
| | 4H-1,2,4-triazol-3-yl)methyl)cyclobutyl)phenyl)- |
| | 4-(trifluoromethyl)isoindolin-1-one |
| e) | 2-(3-(3,3-difluoro-1-((R)-fluoro(4- |
| | methyl-4H-1,2,4-triazol-3- |
| | yl)methyl)cyclobutyl)phenyl)-6-(((S)- |
| | 4-ethyl-2-isopropylpiperazin-1- |
| | yl)methyl)-4-(trifluoromethyl)isoindolin-1-one |
| f) | 2-(3-(3,3-difluoro-1-((S)-fluoro(4- |
| | methyl-4H-1,2,4-triazol-3- |
| | yl)methyl)cyclobutyl)phenyl)-6- |
| | (((S)-4-ethyl-2-isopropylpiperazin-1- |
| | yl)methyl)-4-(trifluoromethyl)isoindolin-1-one |
| g) | 2-(3-(isopropylamino)-5-(1-((4- |
| | methyl-4H-1,2,4-triazol-3- |
| | yl)methyl)cyclobutyl)phenyl)-6-(((1- |
| | methylcyclobutyl)amino)methyl)-4- |
| | (trifluoromethyl)isoindolin-1-one |
| h) | 2-(3-(ethylamino)-5-(1-((4-methyl-4H-1,2,4- |
| | triazol-3-yl)methyl)cyclobutyl)phenyl)-6-(((1- |
| | methylcyclobutyl)amino)methyl)-4- |
| | (trifluoromethyl)isoindolin-1-one |
| i) | (R)-2-(3-(3,3-difluoro-1-(fluoro(4- |
| | methyl-4H-1,2,4-triazol-3- |
| | yl)methyl)cyclobutyl)phenyl)-6-(((1- |
| | methylcyclobutyl)amino)methyl)-4- |
| | (trifluoromethyl)isoindolin-1-one |
| j) | (S)-2-(3-(3,3-difluoro-1-(fluoro(4- |
| | methyl-4H-1,2,4-triazol-3- |
| | yl)methyl)cyclobutyl)phenyl)-6-(((1- |
| | methylcyclobutyl)amino)methyl)-4- |
| | (trifluoromethyl)isoindolin-1-one |
| a) | 2-(3-(3,3-difluoro-1-((4-methyl-4H-1,2,4- |
| | triazol-3-yl)methyl)cyclobutyl)- |
| | 5-(ethylamino)phenyl)-6-(((1- |
| | methylcyclobutyl)amino)methyl)-4- |
| | (trifluoromethyl)isoindolin-1-one |
| b) | (1R,3R)-3-((4-methyl-4H-1,2,4- |
| | triazol-3-yl)methyl)-3-(3-(6-(((1- |
| | methylcyclobutyl)amino)methyl)-1- |
| | oxo-4-(trifluoromethyl)isoindolin-2- |
| | yl)phenyl)cyclobutanecarbonitrile |
| c) | (1S,3S)-3-((4-methyl-4H-1,2,4- |
| | triazol-3-yl)methyl)-3-(3-(6-(((1- |
| | methylcyclobutyl)amino)methyl)-1- |
| | oxo-4-(trifluoromethyl)isoindolin-2- |
| | yl)phenyl)cyclobutanecarbonitrile |
| d) | 2-(3-(3,3-difluoro-1-((4-methyl-4H-1,2,4-triazol-3- |
| | yl)methyl)cyclobutyl)phenyl)-6- |
| | ((2R,5S)-5-ethylpyrrolidin-2-yl)-4- |
| | (trifluoromethyl)isoindolin-1-one |
| e) | (R)-4-chloro-2-(3-(3-(fluoro(4-methyl-4H-1,2,4- |
| | triazol-3-yl)methyl)oxetan-3-yl)phenyl)-6-(((1- |
| | methylcyclobutyl)amino)methyl)isoindolin-1-one |
| f) | (R)-4-bromo-2-(3-(3-(fluoro(4-methyl-4H-1,2,4- |
| | triazol-3-yl)methyl)oxetan-3-yl)phenyl)-6-(((1- |
| | methylcyclobutyl)amino)methyl)isoindolin-1-one |
| g) | 2-(3-cyclopropyl-5-(3-((4-methyl- |
| | 4H-1,2,4-triazol-3-yl)methyl)oxetan-3-yl)phenyl)- |
| | 6-(((1-methylcyclobutyl)amino)methyl)-4- |
| | (trifluoromethyl)isoindolin-1-one |
| h) | 2-(3-ethoxy-5-(3-((4-methyl-4H- |
| | 1,2,4-triazol-3-yl)methyl)oxetan-3-yl)phenyl)- |
| | 6-(((1-methylcyclobutyl)amino)methyl)-4- |
| | (trifluoromethyl)isoindolin-1-one |
| i) | 2-(3-(ethylamino)-5-(3-((4-methyl-4H- |
| | 1,2,4-triazol-3-yl)methyl)oxetan-3-yl)phenyl)- |
| | 6-(((1-methylcyclobutyl)amino)methyl)-4- |
| | (trifluoromethyl)isoindolin-1-one |
| j) | 2-(3-(3,3-difluoro-1-((4-methyl-4H-1,2,4- |
| | triazol-3-yl)methyl)cyclobutyl)phenyl)-6- |
| | ((2S,5S)-5-ethylpyrrolidin-2-yl)-4- |
| | (trifluoromethyl)isoindolin-1-one |
| a) | 2-(3-(3,3-difluoro-1-((4-methyl-4H-1,2,4- |
| | triazol-3-yl)methyl)cyclobutyl)phenyl)-6- |
| | ((2R,5R)-5-ethylpyrrolidin-2-yl)-4- |
| | (trifluoromethyl)isoindolin-1-one |
| b) | 2-(3-(cyclopentylamino)-5-(3,3-difluoro- |
| | 1-((4-methyl-4H-1,2,4-triazol-3- |
| | yl)methyl)cyclobutyl)phenyl)-6-(((1- |
| | methylcyclobutyl)amino)methyl)-4- |
| | (trifluoromethyl)isoindolin-1-one |
| c) | 2-(3-(cyclopentyloxy)-5-(3,3-difluoro- |
| | 1-((4-methyl-4H-1,2,4-triazol-3- |
| | yl)methyl)cyclobutyl)phenyl)-6-(((1- |
| | methylcyclobutyl)amino)methyl)-4- |
| | (trifluoromethyl)isoindolin-1-one |
| d) | 2-(3-(cyclopentyloxy)-5-(3- |
| | ((4-methyl-4H-1,2,4-triazol-3- |
| | yl)methyl)oxetan-3-yl)phenyl)-6-(((1- |
| | methylcyclobutyl)amino)methyl)-4- |
| | (trifluoromethyl)isoindolin-1-one |
| e) | 2-(3-(cyclopentylamino)-5-(3- |
| | ((4-methyl-4H-1,2,4-triazol-3- |
| | yl)methyl)oxetan-3-yl)phenyl)-6-(((1- |
| | methylcyclobutyl)amino)methyl)-4- |
| | (trifluoromethyl)isoindolin-1-one |
| f) | (S)-2-(6-(sec-butylamino)-4-(3- |
| | ((4-methyl-4H-1,2,4-triazol-3- |
| | yl)methyl)oxetan-3-yl)pyridin-2-yl)-4- |
| | (trifluoromethyl)isoindolin-1-one |
| g) | (R)-2-(6-(sec-butylamino)-4-(3- |
| | ((4-methyl-4H-1,2,4-triazol-3- |
| | yl)methyl)oxetan-3-yl)pyridin-2-yl)- |
| | 4-(trifluoromethyl)isoindolin-1-one |
| h) | 2-(6-((3,3-difluorocyclopentyl)amino)-4- |
| | (3-((4-methyl-4H-1,2,4-triazol-3- |
| | yl)methyl)oxetan-3-yl)pyridin-2-yl)-4- |
| | (trifluoromethyl)isoindolin-1-one |
| i) | 2-(6-(cyclopent-3-en-1-ylamino)-4- |
| | (3-((4-methyl-4H-1,2,4-triazol-3- |
| | yl)methyl)oxetan-3-yl)pyridin-2-yl)-4- |
| | (trifluoromethyl)isoindolin-1-one |
| j) | 2-(3-(1-((4-ethyl-4H-1,2,4-triazol- |
| | 3-yl)fluoromethyl)-3,3- |
| | difluorocyclobutyl)phenyl)-6-(((S)- |
| | 2-isopropyl-4-methylpiperazin-1- |
| | yl)methyl)-4-(trifluoromethyl)isoindolin-1-one |
| k) | 2-(3-(3-((R)-(4-cyclopropyl-4H-1,2,4- |
| | triazol-3-yl)fluoromethyl)oxetan-3- |
| | yl)phenyl)-6-(((S)-2-isopropyl-4- |
| | methylpiperazin-1-yl)methyl)-4- |
| | (trifluoromethyl)isoindolin-1-one |
| a) | 2-(3-(3-((S)-(4-cyclopropyl-4H-1,2,4- |
| | triazol-3-yl)fluoromethyl)oxetan-3- |
| | yl)phenyl)-6-(((S)-2-isopropyl-4- |
| | methylpiperazin-1-yl)methyl)-4- |
| | (trifluoromethyl)isoindolin-1-one |
| b) | 2-(4-(3-((4-methyl-4H-1,2,4-triazol- |
| | 3-yl)methyl)oxetan-3-yl)-6-((3,3,3- |
| | trifluoropropyl)amino)pyridin-2-yl)-4- |
| | (trifluoromethyl)isoindolin-1-one |
| c) | 2-(6-((2-hydroxycyclopentyl)amino)- |
| | 4-(3-((4-methyl-4H-1,2,4-triazol-3- |
| | yl)methyl)oxetan-3-yl)pyridin-2-yl)- |
| | 4-(trifluoromethyl)isoindolin-1-one |
| d) | (R)-2-(4-(3-((4-methyl-4H-1,2,4- |
| | triazol-3-yl)methyl)oxetan-3-yl)-6-(3- |
| | methylmorpholino)pyridin-2-yl)-4- |
| | (trifluoromethyl)isoindolin-1-one |
| e) | 2-((4-(3-((4-methyl-4H-1,2,4-triazol-3- |
| | yl)methyl)oxetan-3-yl)-6-(1-oxo-4- |
| | (trifluoromethyl)isoindolin-2- |
| | yl)pyridin-2-yl)amino)acetamide |
| f) | 2-(3-(1-((4-cyclopropyl-4H-1,2,4- |
| | triazol-3-yl)methyl)-3,3- |
| | difluorocyclobutyl)phenyl)-6-(((1- |
| | methylcyclobutyl)amino)methyl)-4- |
| | (trifluoromethyl)isoindolin-1-one |
| g) | 2-(6-(bicyclo[2.1.1]hexan-1-ylamino)- |
| | 4-(3-((4-methyl-4H-1,2,4-triazol-3- |
| | yl)methyl)oxetan-3-yl)pyridin-2-yl)-4- |
| | (trifluoromethyl)isoindolin-1-one |
| h) | (R)-2-(3-(3-(fluoro(4-methyl-4H- |
| | 1,2,4-triazol-3-yl)methyl)oxetan-3-yl)phenyl)- |
| | 6-(((1-methylcyclobutyl)amino)methyl)- |
| | 4-(trifluoromethyl)-isoindolin-1-one |

The disclosure further comprises a method of treating a cancer, comprising administering to a subject in need thereof, a therapeutically effective amount of a compound of formula (I-A), (I-B), (I-C), (I-D), (I-E), (I-f), or (I-G), or a pharmaceutically acceptable salt or solvate thereof.

### Definitions

It is to be noted that the term “a” or “an” object may refer to one or more of that object. As such, the terms “a” (or “an”), “one or more,” and “at least one” can be used interchangeably herein.

All technical and scientific terms used herein have the same meaning as those understood respectively to those skilled in the art, unless otherwise specifically defined herein. Efforts have been made to ensure accuracy with respect to numbers used (e.g. amounts, temperature, etc.) but some experimental errors and deviations should be accounted for when reading this disclosure.

Throughout the specification and the claims, the words “comprise,” “comprises,” “comprising” are used in a non-exclusive sense, except where the context requires otherwise. It is to be understood that embodiments described herein include “consisting of” and/or “consisting essentially of” embodiments refer to some aspect as being exclusively defined.

As used herein, the term “about,” when referring to a value is meant to encompass variations of, in some embodiments ±50%, in some embodiments ±20%, in some embodiments ±10%, in some embodiments ±5%, in some embodiments 1%, in some embodiments ±0.5%, and in some embodiments ±0.1% from the specified amount, as such variations are appropriate to perform the disclosed methods or employ the disclosed compositions.

Where a range of values is provided and unless the context clearly dictates otherwise, it is understood that each intervening value, to the tenth of the unit of the lower limit, between the upper and lower limit of the range, is also disclosed, and that any other stated or intervening value in that stated range, is encompassed within the invention. The upper and lower limits of these small ranges which may independently be included in the smaller rangers is also encompassed within the invention, subject to any specifically excluded limit in the stated range. Where the stated range includes one or both of the limits, ranges excluding either or both of those included limits are also included in the invention.

Chemical structures are depicted herein according to customary display principles of organic chemistry: specifically, carbon atoms are shown only with bonds to non-hydrogen atoms; hydrogen atoms bonded to carbon atoms are not shown (except where necessary to specify stereochemistry). Correspondingly, when considering alternative substituents or groups occupying specific positions in a formula, it is to be assumed that a sufficient number hydrogen atoms (if otherwise unspecified) are present so that ordinary valences are fulfilled. An ordinary valence of carbon is 4.

“Geminal” refers to the relationship between two moieties that are attached to the same atom. For example, in the moiety —CH₂—CR^{x}R^{y}—, R^{x} and R^{y} are geminal to one another, and R^{x} may be referred to as a geminal R group to R^{y}.

“Vicinal” refers to the relationship between two moieties that are attached to adjacent atoms. For example, in the residue —CHR^{x}—CHR^{y}—, R^{x} and R^{y} are vicinal and R^{x} may be referred to as a vicinal R group to R^{y}.

“Optionally substituted” unless otherwise specified means that a group may be unsubstituted, or it may be substituted by one or more (e.g., 1, 2, 3, 4 or 5) non-hydrogen atoms or monovalent groups, such that the substituents may be the same or different from one another. In one embodiment, a group that is optionally substituted has one substituent. In another embodiment, a group that is optionally substituted has two substituents. In another embodiment, a group that is optionally substituted group has three substituents. In another embodiment, a group that is optionally substituted group has four substituents. In some embodiments, a group that is optionally substituted group has 1 to 2, 1 to 3, 1 to 4 or 1 to 5 substituents. When a group includes an atom (e.g., a ring carbon atom, or a terminal methyl group) that can itself accept more than one substituent, then “optionally substituted” as it applies to that group includes groups in which one atom is substituted with two or more substituents as applicable.

Heteroatom refers to any atom other than carbon or hydrogen. Typical heteroatoms found in small organic molecules are selected from: nitrogen, oxygen, fluorine, phosphorous, sulfur, chlorine, and bromine. It is to be understood that, where a heteroatom is specified as a possible member of a ring (or in another bivalent context), then monovalent atoms such as the halogens are excluded in that instance.

“Alkyl” as used herein refers to a saturated linear (i.e., unbranched) or branched univalent hydrocarbon functional group derived by the removal of one hydrogen atom from one carbon atom of a parent alkane. An alkyl group having n carbon atoms, as a radical, has formula CₙH₂ₙ₊₁. Alkyl groups having a given number of carbon atoms can be designated as follows: Cₙ-alkyl to denote any alkyl radical having n carbon atoms, or Cₙ₁₋ₙ₂-alkyl to denote any alkyl radical having from n1 to n2 carbon atoms. Thus C₁₋₁₀ means any alkyl radical having from one to ten carbon atoms. Particular alkyl groups of interest herein are those having 1 to 20 carbon atoms (a “C₁₋₂₀-alkyl”), those having 1 to 12 carbon atoms (a “C₁₋₁₂-alkyl”), those having 1 to 6 carbon atoms (a “C₁₋₆-alkyl”), having 2 to 6 carbon atoms (a “C₂₋₆-alkyl”), or having 1 to 4 carbon atoms (a “C₁₋₄-alkyl”). Examples of alkyl groups include, but are not limited to: methyl, ethyl, n-propyl, isopropyl, n-butyl, t-butyl, isobutyl, sec-butyl. It is to be understood that an alkyl group can bond to another group or moiety at any carbon atom in its structure: thus, for example, butan-1-yl (n-butyl) and butan-2-yl (sec-butyl) are contemplated by the definition herein.

Alkylamino refers to an amino group that has at least one alkyl substituent. Dialkylamino is a special case of alkylamino.

“Alkenyl” as used herein refers to an unsaturated linear (i.e., unbranched) or branched univalent hydrocarbon functional group having at least one site of olefinic unsaturation, i.e., having at least one instance of a carbon-carbon double bond (represented by the formula C═C), and having the number of carbon atoms designated. An alkenyl group having n carbon atoms and a single double-bond, as a radical, has formula CₙH₂ₙ₋₁ and is derived by the removal of one hydrogen atom from one carbon atom of a parent alkene. Alkenyl groups having a given number of carbon atoms can be designated as follows: Cₙ-alkenyl to denote any alkenyl radical having n carbon atoms, or Cₙ₁₋ₙ₂-alkenyl to denote any alkenyl radical having from n1 to n2 carbon atoms. Thus, C₂₋₁₀-alkenyl means an alkenyl group having from two to ten carbon atoms. An alkenyl group may contain constituent carbon atoms that are in “cis” or “trans” configurations, or “E” or “Z” configurations, with respect to a given double bond. Particular alkenyl groups are those having 2 to 20 carbon atoms (a “C₂₋₂₀-alkenyl”), having 2 to 8 carbon atoms (a “C₂₋₈-alkenyl”), having 2 to 6 carbon atoms (a “C₂₋₆-alkenyl”), or having 2 to 4 carbon atoms (a “C₂₋₄-alkenyl”). Preferred alkenyl groups have one double bond. Other alkenyl groups may have two double bonds (and may be referred to as dienyl). In alkenyl groups having more than one double bond, a pair of double bonds may be separated by one carbon-carbon single bond, in which case the arrangement is referred to as “conjugated”, or they may be separated by more than one carbon-carbon single bond. Examples of alkenyl group include, but are not limited to, groups such as ethenyl (or vinyl), prop-1-enyl, prop-2-enyl (or allyl), 2-methylprop-1-enyl, but-1-enyl, but-2-enyl, but-3-enyl, buta-1,3-dienyl, 2-methylbuta-1,3-dienyl, homologs and isomers thereof, and the like.

“Alkynyl” as used herein refers to an unsaturated linear (i.e., unbranched) or branched univalent hydrocarbon having at least one site of acetylenic unsaturation, i.e., having at least one instance of a carbon-carbon triple bond (represented by the formula formula C≡C) and having the number of carbon atoms designated. An alkynyl group having n carbon atoms and a single triple-bond, as a radical, has formula CₙH₂ₙ₋₃, and is derived by the removal of one hydrogen atom from one carbon atom of a parent alkyne. Alkynyl groups having a given number of carbon atoms can be designated as follows: Cₙ-alkynyl to denote any alkynyl radical having n carbon atoms, or Cₙ₁₋ₙ₂-alkynyl to denote any alkynyl radical having from n1 to n2 carbon atoms. Particular alkynyl groups are those having 2 to 20 carbon atoms (a “C₂₋₂₀-alkynyl”), having 2 to 8 carbon atoms (a “C₂₋₈-alkynyl”), having 2 to 6 carbon atoms (a “C₂₋₆-alkynyl”), or having 2 to 4 carbon atoms (a “C₂₋₄-alkynyl”). Examples of alkynyl groups include, but are not limited to, groups such as ethynyl (or acetylenyl), prop-1-ynyl, prop-2-ynyl (or propargyl), but-1-ynyl, but-2-ynyl, but-3-ynyl, homologs and isomers thereof, and the like.

“Alkylenyl” as used herein refers to a saturated linear (i.e., unbranched) or branched bivalent hydrocarbon group having the number of carbon atoms designated. An alkylene group having n carbon atoms, as a radical, has formula —CₙH₂ₙ—. Particular alkylene groups are those having 1 to 6 carbon atoms (a “C₁₋₆-alkylene”), 1 to 5 carbon atoms (a “C₁₋₅-alkylene”), having 1 to 4 carbon atoms (a “C₁₋₄-alkylene”), or 2 to 3 carbon atoms (a “C₂₋₃-alkylene”). Examples of alkylene radicals include, but are not limited to, methylene (—CH₂—), ethylene (—CH₂—CH₂—), propylene (—CH₂—CH₂—CH₂—), butylene (—CH₂—CH₂—CH₂—CH₂—), sec-butylene (—CH(CH₃)—CH₂—CH₂—) and the like.

Cyclic (ring-containing) moieties comprise atoms bonded together in a ring, and have one or more substituents other than hydrogen atoms bonded to one or more ring atoms. Each atom in the ring defines a vertex of a polygon. A cyclic radical, denoted cyclyl, is derived by the removal of one hydrogen atom from one ring atom.

Cyclic moieties may be carbocyclic or heterocyclic. Cyclic moieties include monocyclic, fused ring systems, spiro-ring systems, and bridged ring systems.

Two ring atoms are adjacent to one another in a ring if they are bonded to one another in that same ring. In rings having 4 or more ring atoms, adjacent atoms are bonded to one another but to no other atom in the same ring. In a three-membered ring, each atom is necessarily bonded to each other atom in the ring. Two adjacent ring atoms define one “edge” of the ring.

Two or more cyclic moieties may join to one another in one of several ways to form ring systems that comprise more than one ring. Bicyclic ring systems are those that contain two or more rings that are joined together.

Two rings are fused to one another if two ring atoms are adjacent to one another in both rings and are common to both rings. Such rings are said to share an “edge”.

Spirocyclic ring systems comprise a pair of rings that share a single vertex. Such systems contain a ring junction at which the two rings share a single ring atom. Spirocyclic ring systems may contain one or more heteroatoms as ring atoms.

Bridged ring systems contain at least a pair of rings in which two or more non-adjacent ring atoms are shared by two or more rings. The two non-adjacent ring atoms in question are referred to as “bridgehead” atoms and the pair of bridgehead atoms are members of three different rings, even though the simplest such ring systems are typically referred to as “bridged bicyclic rings”. Examples of carbocyclic radicals containing bridged bicyclic rings are norbornyl and adamantyl. Bridged bicyclic ring systems may contain one or more heteroatoms as ring atoms.

Chained ring systems contain two rings that are joined to one another but do not share any ring atom in common: one ring is a substituent of the other, and vice versa. Biphenyl is an example of a chained ring system.

Ring systems may contain pairs of rings that are fused or chained to one another, spiro-joined, or bridged, or in the case of three or more rings, joined in combinations of ways thereof.

“Carbocycle” as used herein refers to aromatic, saturated or unsaturated cyclic univalent hydrocarbon groups having the number of annular (i.e., ring) carbon atoms designated (i.e., C₃₋₁₀ means three to ten annular carbon atoms). Carbocyclic groups have a single ring (“monocycles”) or more than one ring (“bicycles”, “tricycles”, or polycycles, more generally). Two or more carbocyclic rings may be joined to one another by fused, spiro, bridged, or chained connections as further described elsewhere herein.

It is intended herein that the term carbocycle encompasses radicals having one or more adjacent pairs of ring atoms between which are double bonds, and that, where more than one such double bond is present, the double bonds may or may not form a conjugated system within the ring. Thus carbocycles may be more specifically designated according to whether they are fully saturated (“cycloalkyl”), unsaturated at least in part (“cycloalkenyl”), or fully conjugated, (“aromatic” or “aryl”). Cycloalkyl groups are fully saturated radicals and are derived by the removal of one hydrogen atom from one carbon atom of a parent cycloalkane. Particular cycloalkyl groups are those having from 3 to 12 annular carbon atoms (C₃₋₁₂-cycloalkyl). A preferred cycloalkyl is a monocyclic hydrocarbon having from 3 to 8 annular carbon atoms (a “C₃₋₈-cycloalkyl”), or having 3 to 6 carbon atoms (a “C₃₋₆-cycloalkyl”). Single ring cycloalkyl radicals have formula CₙH₂ₙ₋₁. Examples of cycloalkyl groups include, but are not limited to, cyclopropyl, cyclobutyl, cyclopentyl, cyclohexyl, and cycloheptyl. Cycloalkenyl groups have one or more double bonds between adjacent ring carbon atoms. Examples of cycloalkenyl groups include 1-cyclohex-1-enyl, and 1-cyclohex-3-enyl.

“Aryl” as used herein refers to a carbocyclic group having a aromatic single ring (e.g., phenyl) or multiple aromatic rings fused to one another (e.g., naphthyl). Preferably, an aryl group comprises from 6 to 20 carbon atoms, more preferably between 6 to 12 carbon atoms. Particularly preferred aryl groups are those having from 6 to 14 annular carbon atoms (a “C₆₋₁₄-aryl”). The term aromatic is used herein as it is typically used in organic chemistry, meaning, with a few understood exceptions, rings and ring systems in which the annular atoms contribute a total of (4n+2) pi electrons to a set of delocalized molecular orbitals, where n is a non-zero positive integer.

Typical aryl groups include, but are not limited to, groups derived from fused ring systems that comprise one or more aromatic rings, or conjugated ring systems, such as but not limited to aceanthrylene, acenaphthylene, acephenanthrylene, anthracene, azulene, benzene, chrysene, coronene, fluoranthene, heptaphene, hexacene, hexaphene, as-indacene, s-indacene, indene, naphthalene (hexalene), octacene, octaphene, octalene, ovalene, pentacene, pentalene, pentaphene, perylene, phenalene, phenanthrene, picene, pleiadene, pyrene, pyranthrene, rubicene, tetraphenylene, triphenylene, and trinaphthalene.

A “heterocyclic”, or “heterocyclyl”, group as used herein refers to a saturated or an unsaturated but non-aromatic, cyclic group having one or more rings that comprises at least one carbon atom and one or more heteroatoms. Typically such a ring has from 1 to 14 ring carbon atoms and from 1 to 6 ring heteroatoms that can be same or different from each other. Such a group is typically derived by the removal of one hydrogen atom from one ring atom of a parent heterocycle. Therefore a heterocyclyl group can bond to a position on a scaffold through either a ring carbon atom or a ring heteroatom such as a nitrogen atom. It is intended herein that the term heterocyclyl encompasses radicals having one or more double bonds between adjacent ring atoms, and that where more than one such double bond is present, the double bonds do not form a conjugated system within the ring.

Particularly preferred heterocyclyl groups are: 3- to 14-membered rings having 1 to 13 annular carbon atoms and 1 to 6 annular heteroatoms independently selected from nitrogen, phosphorus, oxygen and sulfur; 3- to 12-membered rings having 1 to 11 annular carbon atoms and 1 to 6 annular heteroatoms independently selected from nitrogen, phosphorus, oxygen and sulfur; 3- to 10-membered rings having 1 to 9 annular carbon atoms and 1 to 4 annular heteroatoms independently selected from nitrogen, phosphorus, oxygen and sulfur; 3- to 8-membered rings having 1 to 7 annular carbon atoms and 1 to 4 annular heteroatoms independently selected from nitrogen, phosphorus, oxygen and sulfur; and 3- to 6-membered rings having 1 to 5 annular carbon atoms and 1 to 4 annular heteroatoms independently selected from nitrogen, phosphorus, oxygen and sulfur. In one variation, heterocyclyl include monocyclic 3-, 4-, 5-, 6- or 7-membered rings having from 1 to 2, 1 to 3, 1 to 4, 1 to 5 or 1 to 6 annular carbon atoms and 1 to 2, 1 to 3 or 1 to 4 annular heteroatoms independently selected from from nitrogen, phosphorus, oxygen and sulfur. In another variation, heterocyclyl includes polycyclic non-aromatic rings having from 1 to 12 annular carbon atoms and 1 to 6 annular heteroatoms independently selected from nitrogen, phosphorus, oxygen and sulfur. Exemplary heterocyclic rings include: aziridine, azetidine, pyrrolidine, piperazine, piperidine, oxetane, tetrahydrofuran, and morpholine.

A heterocyclic ring may make fused, spiro, or bridged, connections or make any combination of such connections to one or more other rings.

“Heteroaryl” or “heteroaromatic”, as used herein, refers to an aromatic cyclic group having from 1 to 14 ring carbon atoms and at least one ring heteroatom, including but not limited to heteroatoms such as nitrogen, phosphorus, oxygen and sulfur. The term refers to a monovalent heteroaromatic radical derived by the removal of one hydrogen atom from a single ring atom of a parent heteroaromatic ring system. A heteroaryl group may have a single ring (e.g., pyridyl, furyl) or multiple fused rings (e.g., indolizinyl, benzothienyl). Particular heteroaryl groups are 5- to 14-membered rings having 1 to 12 annular (i.e., ring) carbon atoms and 1 to 6 annular (i.e., ring) heteroatoms independently selected from nitrogen, phosphorus, oxygen and sulfur; 5- to 10-membered rings having 1 to 8 annular carbon atoms and 1 to 4 annular heteroatoms independently selected from nitrogen, phosphorus, oxygen and sulfur; and 5-, 6- or 7-membered rings having 1 to 5 annular carbon atoms and 1 to 4 annular heteroatoms independently selected from nitrogen, oxygen and sulfur In one variation, heteroaryl include monocyclic aromatic 5-, 6- or 7-membered rings having from 1 to 6 annular carbon atoms and 1 to 4 annular heteroatoms independently selected from nitrogen, oxygen and sulfur. In another variation, heteroaryl includes polycyclic aromatic rings having from 1 to 12 annular carbon atoms and 1 to 6 annular heteroatoms independently selected from nitrogen, phosphorus, oxygen and sulfur.

Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. Preferred heteroaryl groups are those derived from thiophene, pyrrole, benzothiophene, benzofuran, indole, pyridine, quinoline, imidazole, oxazole and pyrazine.

Rings of different categories may be connected to one another, such as by fused, spiro, or bridged, connections, or by combinations thereof. Such a ring system can be referred to as a “mixed” ring system.

For example, at least one ring of a multiple ring system can be aromatic on its own, though one or more of the remaining fused rings may be not aromatic. Examples of fused ring systems that contain at least one aromatic ring and at least one partially saturated ring include fluorene, indane, and biphenylene.

A mixed ring system having more than one ring where at least one ring is aromatic and at least one ring is non-aromatic may be connected to another structure by bonding to either an aromatic ring atom or a non-aromatic ring atom.

A heteroaryl group having more than one ring where at least one ring is non-aromatic may be connected to another structure at either an aromatic ring position or at a non-aromatic ring position.

Similarly, carbocyclic and heterocyclic groups may join to one another in one of several ways to form ring systems that comprise more than one ring.

“Halogen” refers to an atom selected from fluorine, chlorine, bromine and iodine. The terms “halide” or “halo” refer to halogens as substituents, in which each is individually referred to as fluoro, chloro, bromo, and/or iodo, or as “fluoride”, “chloride”, “bromide”, or “iodide”. An alkyl group in which one or more hydrogen atoms is each replaced by a halogen atom is referred to as a “haloalkyl”. For example, “C₁₋₆-haloalkyl” refers to an alkyl group having from 1-6 carbon atoms in which at least one hydrogen atom is replaced by a halogen atom. Where a moiety is substituted with more than one instance of a given halogen atom, it may be referred to by using a prefix corresponding to the number of halogen moieties attached, e.g., dihaloaryl, trihaloaryl, refer to aryl groups substituted with two (“di”) or three (“tri”) halo groups respectively. It is to be understood that, where more than one halo groups are present they are not necessarily the same as one another. Thus 4-chloro-3-fluorophenyl is within the scope of dihaloaryl. An alkyl group in which every hydrogen is replaced with a halogen atom is referred to as a “perhaloalkyl.” A preferred perhaloalkyl group is trifluoromethyl (—CF₃). Similarly, “perhaloalkoxy” refers to an alkoxy group in which a halogen takes the place of each H in the hydrocarbon making up the alkyl moiety of the alkoxy group. An example of a perhaloalkoxy group is trifluoromethoxy (—OCF₃).

“Carbonyl” refers to the group C═O.

“Thiocarbonyl” refers to the group C═S.

“Oxo” refers to the moiety=O, i.e., an oxygen atom double-bonded to a second atom other than oxygen. A carbon atom in a chain or a ring that is bonded to an oxo moiety is also referred to as a carbonyl group. A sulfur atom in a chain or a ring can accept two oxo substituents.

“Prodrug” refers to a pharmacologically inactive derivative of a drug molecule that requires a transformation within the body, usually a metabolic transformation, to release the active drug.

“Promoiety” refers to a form of protecting group that, when used to mask a functional group within a drug molecule converts the drug into a prodrug. Typically, the promoiety will be attached to the drug via bond(s) that are cleaved by enzymatic or non-enzymatic means in vivo. Ideally, the promoiety is rapidly cleared from the body upon cleavage from the prodrug.

“Protecting group” refers to a grouping of atoms that when attached to a reactive group in a molecule masks, reduces or prevents that reactivity. Examples of protecting groups can be found in Green et al., “Protective Groups in Organic Chemistry”, (Wiley, 2nd ed. 1991) and Harrison et al., “Compendium of Synthetic Organic Methods”, Vols. 1-8 (John Wiley and Sons, 1971-1996). Representative amino protecting groups include, but are not limited to, formyl, acetyl, trifluoroacetyl, benzyl, benzyloxycarbonyl“(“CBZ”), tert-butoxycarbonyl (“Boc”), trimethylsilyl (“TMS”), 2-trimethylsilyl-ethanesulfonyl (“SES”), trityl and substituted trityl groups, allyloxycarbonyl, 9-fluorenylmethyloxycarbonyl (“FMOC”), nitro-veratryloxycarbonyl (“NVOC”) and the like. Representative hydroxy protecting groups include, but are not limited to, those where the hydroxy group is either acylated or alkylated such as benzyl, and trityl ethers as well as alkyl ethers, tetrahydropyranyl ethers, trialkylsilyl ethers and allyl ethers.

Compounds of Formula (I-A), (I-B), (I-C), (I-D), (I-E), (I-F), and (I-G), collectively “Formulae (I-A)-(I-G)”, described herein, or a salt or solvate thereof may exist in stereoisomeric forms (e.g., such a compound contains one or more asymmetric carbon atoms). The individual stereoisomers (such as purified enantiomers and diastereomers) and mixtures of these or enantiomerically/diastereomerically enriched mixtures are included within the scope of the subject matter disclosed herein.

It is further understood that a compound or salt of Formulae (I-A)-(I-G) may exist in tautomeric forms other than that shown in the formula and these are also included within the scope of the subject matter disclosed herein.

The subject matter disclosed herein also includes isotopically-labelled forms of the compounds described herein, i.e., compounds that have the formulae shown herein but for the fact that one or more constituent atoms are replaced by an atom having an atomic mass or mass number different from the atomic mass or mass number usually found in nature and/or at an abundance not normally found in nature. Examples of isotopes that can be incorporated into compounds described herein and pharmaceutically acceptable salts thereof, at levels that differ from the natural distribution of such isotopes, include isotopes of hydrogen, carbon, nitrogen, oxygen, phosphorous, sulphur, fluorine, iodine, and chlorine, such as ²H, ³H, ¹¹C, ¹³C, ¹⁴C, ¹⁵N, ¹⁷O, ¹⁸O, ³¹P, ³²P, ³⁵S, ¹⁸F, ³⁶Cl, ¹²³I and ¹²⁵I.

The subject matter disclosed herein further includes prodrugs, metabolites, and pharmaceutically acceptable salts of compounds of Formulae (I-A)-(I-G). Metabolites of the compounds of Formulae (I-A)-(I-G) include compounds produced by a process comprising contacting a compound of Formulae (I-A)-(I-G) with a mammal for a period of time sufficient to yield a metabolic product thereof.

In some embodiments, the salts of the compounds of the invention are pharmaceutically acceptable salts. “Pharmaceutically acceptable salts” are those salts that retain at least some of the biological activity of the free (non-salt) compound and that can be administered as drugs or pharmaceuticals to a subject. Such salts, for example, include: (1) acid addition salts; (2) salts formed when an acidic proton is replaced by a metal ion; or (3) an acidic proton coordinates with an organic base. Pharmaceutically acceptable salts can be prepared in situ in the manufacturing process, or by separately reacting a purified compound of the invention in its free acid or base form with a suitable organic or inorganic base or acid respectively, and isolating the salt thus formed during subsequent purification.

If the compound of Formulae (I-A)-(I-G) is a base, the desired pharmaceutically acceptable salt may be prepared as an acid addition salt by any suitable method available in the art, for example, treatment of the free base with an inorganic acid, such as hydrochloric acid, hydrobromic acid, sulfuric acid, nitric acid, methanesulfonic acid, phosphoric acid and others of like property, or with an organic acid, such as acetic acid, maleic acid, succinic acid, mandelic acid, fumaric acid, propionic acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, and others of like property.

If the compound of Formulae (I-A)-(I-G) is an acid, one or more acidic protons present may be replaced by a metal ion, e.g., an alkali metal ion, an alkaline earth ion, or an aluminum ion. The desired pharmaceutically acceptable salt may then be prepared by any suitable method, for example, treatment of the free acid with an inorganic or organic base, such as an amine (primary, secondary or tertiary), an alkali metal hydroxide or alkaline earth metal hydroxide, or the like. Examples of suitable salts include, but are not limited to, organic salts derived from amino acids, such as glycine and arginine, ammonia, and cyclic amines, such as piperidine, morpholine and piperazine, alcoholamines such as ethanolamine, diethanolamine, and triethanolamine, and inorganic salts derived from sodium, calcium, potassium, magnesium, manganese, iron, copper, zinc, aluminum and lithium. Acceptable inorganic bases include aluminum hydroxide, calcium hydroxide, potassium hydroxide, sodium carbonate, sodium hydroxide, and the like.

The compounds herein may also be present as solvates, such as crystallized with a corresponding quantity of a solvent molecule, in a ratio that may or may not be stoichiometric. In preferred embodiments the solvent is water, in which case the solvate is a hydrate. In preferred embodiments, one or more solvent molecules are present in stoichiometric ratios relative to molecules of the compound.

A compound of Formulae (I-A)-(I-G) can also be in the form of a “prodrug,” which includes compounds with moieties that can be metabolized in vivo. Generally, prodrugs are metabolized in vivo by esterases or by other mechanisms to form active drugs inside the patient's body. Examples of prodrugs and their uses are well known in the art (see, e.g., Berge et al. (1977) “Pharmaceutical Salts”, J. Pharm. Sci., 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Prodrugs which are converted to active forms through other mechanisms in vivo are also included. In aspects, the compounds of the invention are prodrugs of any of the formulae herein.

It is also to be understood that the subject matter disclosed herein includes combinations and subsets of the particular categories (e.g., salt forms, tautomers, stereoisomeric forms) described herein.

The terms “treat” and “treatment” refer to therapeutic treatment, wherein an object is to slow down, diminish, or attenuate, an undesired physiological change or disorder, such as the development or spread of arthritis or cancer. For purposes of this disclosure, beneficial or desired clinical results include, but are not limited to: alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to a patient's expected survival if not receiving treatment. Those in need of treatment include those with the condition or disorder, and further include those who are only experiencing an early stage of the disorder or disease, in which one or more typical symptoms may yet to manifest.

The phrase “therapeutically effective amount” means an amount of a compound of the present invention, or a salt thereof, that is sufficient to produce a desired therapeutic outcome. Such an amount is sufficient to (i) treat the particular disease, condition, or disorder, (ii) attenuate, ameliorate, or eliminate one or more symptoms (such as biochemical, histologic and/or behavioral) of the particular disease, condition, or disorder, (iii) prevent or delay the onset of one or more symptoms of the particular disease, condition, or disorder described herein, or (iv) reduce the severity or duration of, stabilize the severity of, or eliminate one or more symptoms of the disease, condition or disorder. Other beneficial or desired results of a therapeutic use include, e.g., decreasing one or more symptoms resulting from the disease, including its complications and intermediate pathological phenotypes presenting during development of the disease, increasing the quality of life of those suffering from the disease, decreasing the dose of other medications required to treat the disease, enhancing effect of another medication, delaying the progression of the disease, and/or prolonging survival of patients. In the case of a cancer, the therapeutically effective amount of the drug may reduce the number of cancer cells; reduce a tumor size or check its rate of growth; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, for example, by assessing the time to disease progression (TTP) and/or determining the response rate (RR). In various embodiments, the amount is sufficient to ameliorate, palliate, lessen, and/or delay one or more of symptoms of cancer.

The term “cancer” refers to or describe the physiological condition in mammals that is typically characterized by unregulated cell growth and an invasive nature, wherein the cancerous cells are capable of local invasion and/or metastasis to noncontiguous sites. As used herein, “cancer cells,” “cancerous cells,” or “tumor cells” refer to the cells that are characterized by this unregulated cell growth and invasive property. A “tumor” comprises more than one cancerous cells. Cancers can further be divided into liquid or solid types. The term “cancer” as used herein generally encompasses all types of cancers, subject to specific context. Examples of cancers include, but are not limited to, carcinoma, melanoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, esophageal cancer, endometrial or uterine carcinoma, salivary gland carcinoma, kidney or renal cancer, prostate cancer, vulval cancer, thyroid cancer, hepatic carcinoma, anal carcinoma, penile carcinoma, Ewing's sarcoma, medulloblastomer, as well as head and neck cancer.

A “chemotherapeutic agent” is a chemical compound or biologic useful in the treatment of cancer. A chemotherapeutic agent can be an immunotherapeutic agent. As used herein, an “immunotherapeutic agent” is a compound that enhances the immune system to help fight cancer, specifically or non-specifically. Immunotherapeutics include monoclonal antibodies and non-specific immunotherapies that boost the immune system,

As used herein, a “combination therapy” is a therapy that includes two or more different compounds, administered simultaneously or contemporaneously. Typically, each of the two or more different compounds has a different mechanism of action. Thus, in one aspect, a combination therapy comprising a compound detailed herein and another compound is provided. In some variations, the combination therapy optionally includes one or more pharmaceutically acceptable carriers or excipients, non-pharmaceutically active compounds, and/or inert substances. Combination therapies can comprise two more compounds in a single delivery vehicle such as a tablet, or can comprise doses in separate formulations, such as different tablets, or a tablet and an injectable solution.

As used herein, the term “effective amount” means such an amount of a compound of the invention that, in combination with its parameters of efficacy and toxicity, should be effective in a given administered form. As is understood in the art, an effective amount may be in one or more doses, i.e., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial results may be or is achieved. Suitable doses of any of the co-administered compounds may optionally be lowered due to the combined action (e.g., additive or synergistic effects) of the compounds.

A “prophylactically effective amount” refers to an amount of a compound, or pharmaceutically acceptable salt thereof, sufficient to prevent or reduce the severity of one or more future symptoms of a disease or disorder when administered to a subject who is susceptible and/or who may develop the disease or disorder. For prophylactic use, beneficial or desired results include, e.g., results such as eliminating or reducing the risk, lessening the severity of future disease, or delaying the onset of the disease (e.g., delaying biochemical, histologic and/or behavioral symptoms of the disease, its complications, and intermediate pathological phenotype presenting during future development of the disease).

It is understood that an effective amount of a compound as disclosed herein, or pharmaceutically acceptable salt thereof, including a prophylactically effective amount, may be given to a subject in the adjuvant setting, which refers to a clinical setting in which a subject has had a history of the disease or disorder, and generally (but not necessarily) has been responsive to therapy, which includes, but is not limited to, surgery (e.g., surgical resection), radiotherapy, and chemotherapy. However, because of their or their family's history of the disease or disorder, these subjects are considered at risk of developing it. Treatment or administration in the “adjuvant setting” refers to a subsequent mode of treatment.

As used herein, “unit dosage form” refers to physically discrete units, suitable as unit dosages, each unit containing a predetermined quantity of active ingredient calculated to produce a desired therapeutic effect, in association with the required pharmaceutical carrier or excipient. Unit dosage forms may contain a single compound or a combination therapy.

As used herein, the term “controlled release” refers to a formulation or fraction thereof containing an active pharmaceutical ingredient in which release of the pharmaceutical is not immediate. Thus, with a “controlled release” formulation, administration to a subject does not result in immediate release of the drug into the subject's circulation. The term encompasses depot formulations designed to gradually release the drug compound over an extended period of time. Controlled release formulations can include a wide variety of drug delivery systems, generally involving mixing the drug compound with carriers, polymers or other compounds having the desired release characteristics (e.g., pH-dependent or non-pH-dependent solubility, different degrees of water solubility, and the like), and formulating the mixture according to the desired route of delivery, (e.g., coated capsules, implantable reservoirs, injectable solutions containing biodegradable capsules, and the like).

As used herein, by “pharmaceutically acceptable” or “pharmacologically acceptable” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared and updated by the U.S. Food and Drug Administration.

The term “excipient” as used herein means an inert or inactive substance that may be used in the production of a drug or pharmaceutical, such as a tablet containing a compound of the invention as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, or xanthan gum. Coatings include, e.g., cellulose acetate phthalate, ethylcellulose, gellan gum, maltodextrin, enteric coatings. Compression/encapsulation aids include, e.g., calcium carbonate, dextrose, fructose dc (dc means “directly compressible”), honey dc, lactose (anhydrate or monohydrate; optionally in combination with aspartame, cellulose, or microcrystalline cellulose), starch dc, sucrose. Disintegrants include, e.g., croscarmellose sodium, gellan gum, sodium starch glycolate. Creams or lotions include, e.g., maltodextrin, carrageenans. Lubricants include, e.g., magnesium stearate, stearic acid, sodium stearyl fumarate. Materials for chewable tablets include, e.g. dextrose, fructose dc, lactose (monohydrate, optionally in combination with aspartame or cellulose). Suspending/gelling agents include, e.g., carrageenan, sodium starch glycolate, xanthan gum. Sweeteners include, e.g., aspartame, dextrose, fructose dc, sorbitol, sucrose dc, etc.; and wet granulation agents include, e.g., calcium carbonate, maltodextrin, microcrystalline cellulose, etc. In some cases, the terms “excipient” and “carrier” are used interchangeably.

The term “subject” or “patient” refers humans, whether adult, juvenile or infant, but may also encompass other higher animals such as mammals, in which case the term may include, but is not limited to, primates, cows, sheep, goats, horses, dogs, cats, rabbits, rats, mice.

Representative lactam (isoindolin-1-one) compounds according to Formulae (I-A)-(I-G) are shown in Table 1. Additional compounds falling within Formulae I-A-I-G are provided in the Examples. It is understood that individual enantiomers and diastereomers are included in the table below, as applicable. It is further to be understood that inclusion of a single particular enantiomer or diastereomer of a particular molecule does not preclude its partner enantiomer, or another stereoisomer thereof, from being encompassed by the current disclosure. The numbering of compounds herein (such as in the first column of Table 1), including both the use or omission of particular numbers or sequences of numbers, is arbitrary.

TABLE 1
Lactams Comprising Isoindolin-1-one and Other Scaffolds

| Compound # | Structure | Name |
|---|---|---|
| 9 | 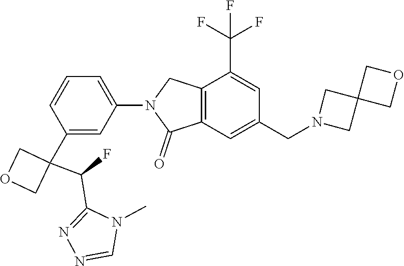 [SMILES: Cn1cnnc1[C@H](F)C1(c2cccc(N3Cc4c(cc(CN5CC6(COC6)C5)cc4C(F)(F)F)C3=O)c2)COC1] | (R)-6-(2-oxa-6-azaspiro[3.3]heptan-6- ylmethyl)-2-(3-(3-(fluoro(4-methyl-4H- 1,2,4-triazol-3-yl)methyl)oxetan-3- yl)phenyl)-4-(trifluoromethyl)isoindolin- 1-one |
| 10 | 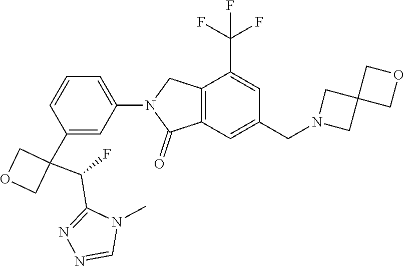 [SMILES: Cn1cnnc1[C@@H](F)C1(c2cccc(N3Cc4c(cc(CN5CC6(COC6)C5)cc4C(F)(F)F)C3=O)c2)COC1] | (S)-6-(2-oxa-6-azaspiro[3.3]heptan-6- ylmethyl)-2-(3-(3-(fluoro(4-methyl-4H- 1,2,4-triazol-3-yl)methyl)oxetan-3- yl)phenyl)-4-(trifluoromethyl)isoindolin- 1-one |
| 11 | 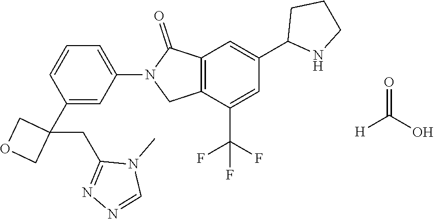 [SMILES: Cn1cnnc1CC1(c2cccc(N3Cc4c(cc(C5CCCN5)cc4C(F)(F)F)C3=O)c2)COC1 ; O=CO] | (±)-2-(3-(3-((4-methyl-4H-1,2,4-triazol- 3-yl)methyl)oxetan-3-yl)phenyl)-6- (pyrrolidin-2-yl)-4- (trifluoromethyl)isoindolin-1-one formate |
| 12 | 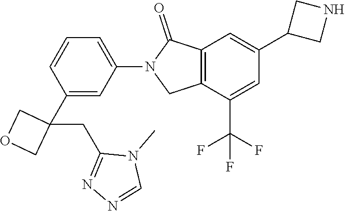 [SMILES: Cn1cnnc1CC1(c2cccc(N3Cc4c(cc(C5CNC5)cc4C(F)(F)F)C3=O)c2)COC1] | 6-(azetidin-3-yl)-2-(3-(3-((4-methyl-4H- 1,2,4-triazol-3-yl)methyl)oxetan-3- yl)phenyl)-4-(trifluoromethyl)isoindolin- 1-one |
| 13 | 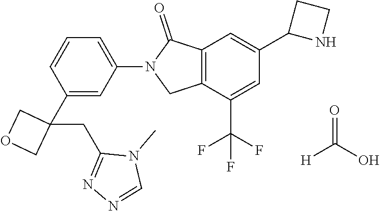 [SMILES: Cn1cnnc1CC1(c2cccc(N3Cc4c(cc(C5CCN5)cc4C(F)(F)F)C3=O)c2)COC1 ; O=CO] | (±)-6-(azetidin-2-yl)-2-(3-(3-((4-methyl- 4H-1,2,4-triazol-3-yl)methyl)oxetan-3- yl)phenyl)-4-(trifluoromethyl)isoindolin- 1-one formate |
| 14 | 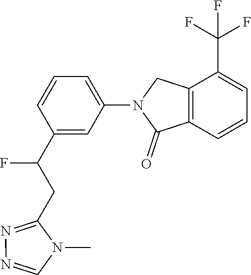 [SMILES: Cn1cnnc1CC(F)c1cccc(N2Cc3c(cccc3C(F)(F)F)C2=O)c1] | (±)-2-(3-(1-fluoro-2-(4-methyl-4H-1,2,4- triazol-3-yl)ethyl)phenyl)-4- (trifluoromethyl)isoindolin-1-one |
| 15 | 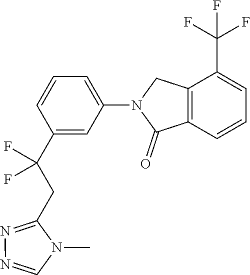 [SMILES: Cn1cnnc1CC(F)(F)c1cccc(N2Cc3c(cccc3C(F)(F)F)C2=O)c1] | 2-(3-(1,1-difluoro-2-(4-methyl-4H-1,2,4- triazol-3-yl)ethyl)phenyl)-4- (trifluoromethyl)isoindolin-1-one |
| 17 | 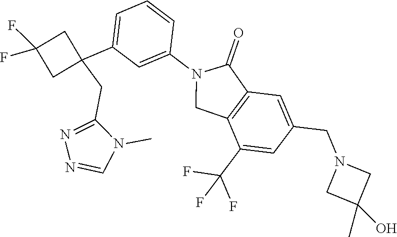 [SMILES: Cn1cnnc1CC1(c2cccc(N3Cc4c(cc(CN5CC(C)(O)C5)cc4C(F)(F)F)C3=O)c2)CC(F)(F)C1] | 2-(3-(3,3-difluoro-1-((4-methyl-4H- 1,2,4-triazol-3- yl)methyl)cyclobutyl)phenyl)-6-((3- hydroxy-3-methylazetidin-1-yl)methyl)- 4-(trifluoromethyl)isoindolin-1-one |
| 27 | 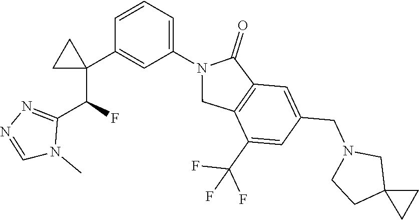 [SMILES: Cn1cnnc1[C@H](F)C1(c2cccc(N3Cc4c(cc(CN5CCC6(CC6)C5)cc4C(F)(F)F)C3=O)c2)CC1] | (R)-6-(5-azaspiro[2.4]heptan-5- ylmethyl)-2-(3-(1-(fluoro(4-methyl-4H- 1,2,4-triazol-3- yl)methyl)cyclopropyl)phenyl)-4- (trifluoromethyl)isoindolin-1-one |
| 28 | 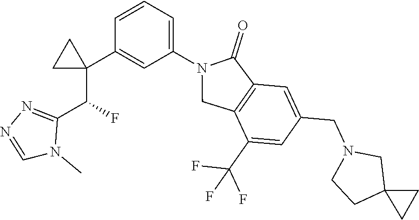 [SMILES: Cn1cnnc1[C@@H](F)C1(c2cccc(N3Cc4c(cc(CN5CCC6(CC6)C5)cc4C(F)(F)F)C3=O)c2)CC1] | (S)-6-(5-azaspiro[2.4]heptan-5- ylmethyl)-2-(3-(1-(fluoro(4-methyl-4H- 1,2,4-triazol-3- yl)methyl)cyclopropyl)phenyl)-4- (trifluoromethyl)isoindolin-1-one |
| 29 | 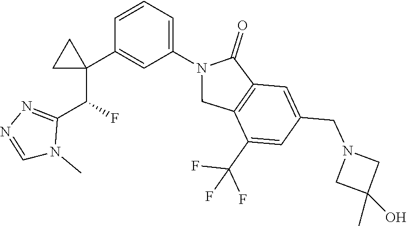 [SMILES: Cn1cnnc1[C@@H](F)C1(c2cccc(N3Cc4c(cc(CN5CC(C)(O)C5)cc4C(F)(F)F)C3=O)c2)CC1] | (S)-2-(3-(1-(fluoro(4-methyl-4H-1,2,4- triazol-3-yl)methyl)cyclopropyl)phenyl)- 6-((3-hydroxy-3-methylazetidin-1- yl)methyl)-4-(trifluoromethyl)isoindolin- 1-one |
| 30 | 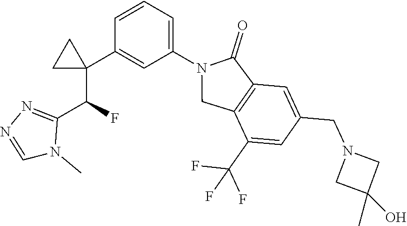 [SMILES: Cn1cnnc1[C@H](F)C1(c2cccc(N3Cc4c(cc(CN5CC(C)(O)C5)cc4C(F)(F)F)C3=O)c2)CC1] | (R)-2-(3-(1-(fluoro(4-methyl-4H-1,2,4- triazol-3-yl)methyl)cyclopropyl)phenyl)- 6-((3-hydroxy-3-methylazetidin-1- yl)methyl)-4-(trifluoromethyl)isoindolin- 1-one |
| 31 | 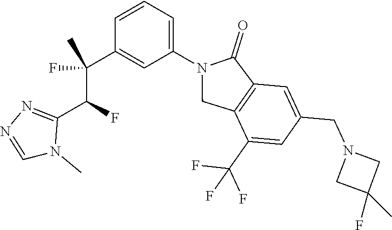 [SMILES: Cn1cnnc1[C@H](F)[C@](C)(F)c1cccc(N2Cc3c(cc(CN4CC(C)(F)C4)cc3C(F)(F)F)C2=O)c1] | 2-(3-((1S,2R)-1,2-difluoro-1-(4-methyl- 4H-1,2,4-triazol-3-yl)propan-2- yl)phenyl)-6-((3-fluoro-3- methylazetidin-1-yl)methyl)-4- (trifluoromethyl)isoindolin-1-one |
| 32 | 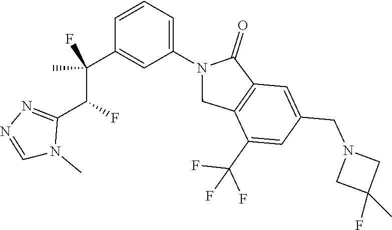 [SMILES: Cn1cnnc1[C@@H](F)[C@@](C)(F)c1cccc(N2Cc3c(cc(CN4CC(C)(F)C4)cc3C(F)(F)F)C2=O)c1] | 2-(3-((1R,2S)-1,2-difluoro-1-(4-methyl- 4H-1,2,4-triazol-3-yl)propan-2- yl)phenyl)-6-((3-fluoro-3- methylazetidin-1-yl)methyl)-4- (trifluoromethyl)isoindolin-1-one |
| 33 | 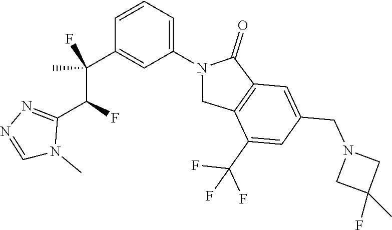 [SMILES: Cn1cnnc1[C@H](F)[C@@](C)(F)c1cccc(N2Cc3c(cc(CN4CC(C)(F)C4)cc3C(F)(F)F)C2=O)c1] | 2-(3-((1S,2S)-1,2-difluoro-1-(4-methyl- 4H-1,2,4-triazol-3-yl)propan-2- yl)phenyl)-6-((3-fluoro-3- methylazetidin-1-yl)methyl)-4- (trifluoromethyl)isoindolin-1-one |
| 34 | 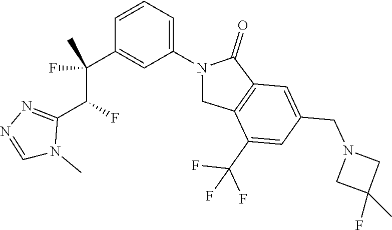 [SMILES: Cn1cnnc1[C@@H](F)[C@](C)(F)c1cccc(N2Cc3c(cc(CN4CC(C)(F)C4)cc3C(F)(F)F)C2=O)c1] | 2-(3-((1R,2R)-1,2-difluoro-1-(4-methyl- 4H-1,2,4-triazol-3-yl)propan-2- yl)phenyl)-6-((3-fluoro-3- methylazetidin-1-yl)methyl)-4- (trifluoromethyl)isoindolin-1-one |
| 47 | 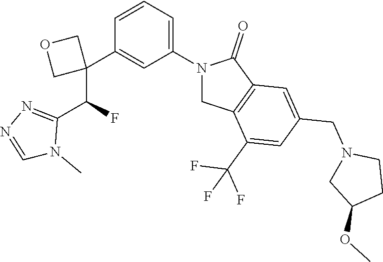 [SMILES: CO[C@@H]1CCN(Cc2cc3c(c(C(F)(F)F)c2)CN(c2cccc(C4([C@@H](F)c5nncn5C)COC4)c2)C3=O)C1] | 2-(3-(3-((R)-fluoro(4-methyl-4H-1,2,4- triazol-3-yl)methyl)oxetan-3-yl)phenyl)- 6-(((R)-3-methoxypyrrolidin-1- yl)methyl)-4-(trifluoromethyl)isoindolin- 1-one |
| 48 | 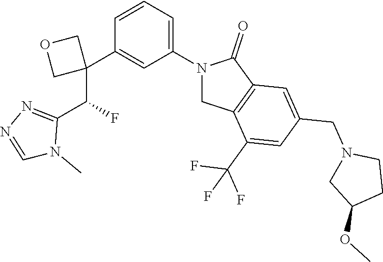 [SMILES: CO[C@@H]1CCN(Cc2cc3c(c(C(F)(F)F)c2)CN(c2cccc(C4([C@H](F)c5nncn5C)COC4)c2)C3=O)C1] | 2-(3-(3-((S)-fluoro(4-methyl-4H-1,2,4- triazol-3-yl)methyl)oxetan-3-yl)phenyl)- 6-(((R)-3-methoxypyrrolidin-1- yl)methyl)-4-(trifluoromethyl)isoindolin- 1-one |
| 49 | 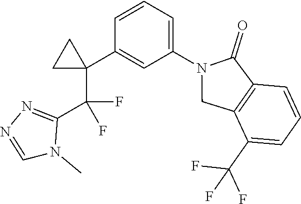 [SMILES: Cn1cnnc1C(F)(F)C1(c2cccc(N3Cc4c(cccc4C(F)(F)F)C3=O)c2)CC1] | 2-(3-(1-(difluoro(4-methyl-4H-1,2,4- triazol-3-yl)methyl)cyclopropyl)phenyl)- 4-(trifluoromethyl)isoindolin-1-one |
| 50 | 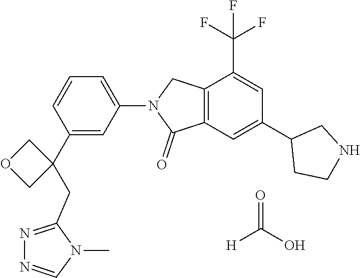 | (±)-2-(3-(3-((4-methyl-4H-1,2,4-triazol- 3-yl)methyl)oxetan-3-yl)phenyl)-6- (pyrrolidin-3-yl)-4- (trifluoromethyl)isoindolin-1-one formate |
| 51 | 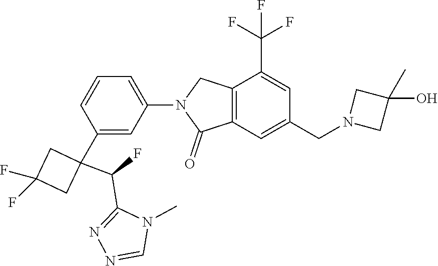 [SMILES: Cn1cnnc1[C@H](F)C1(c2cccc(N3Cc4c(cc(CN5CC(C)(O)C5)cc4C(F)(F)F)C3=O)c2)CC(F)(F)C1] | (R)-2-(3-(3,3-difluoro-1-(fluoro(4- methyl-4H-1,2,4-triazol-3- yl)methyl)cyclobutyl)phenyl)-6-((3- hydroxy-3-methylazetidin-1-yl)methyl)- 4-(trifluoromethyl)isoindolin-1-one |
| 52 | 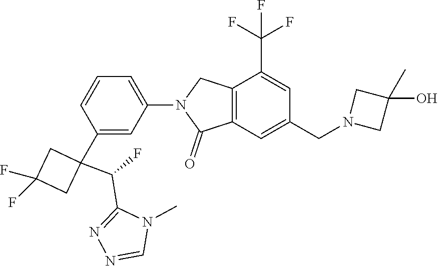 [SMILES: Cn1cnnc1[C@@H](F)C1(c2cccc(N3Cc4c(cc(CN5CC(C)(O)C5)cc4C(F)(F)F)C3=O)c2)CC(F)(F)C1] | (S)-2-(3-(3,3-difluoro-1-(fluoro(4- methyl-4H-1,2,4-triazol-3- yl)methyl)cyclobutyl)phenyl)-6-((3- hydroxy-3-methylazetidin-1-yl)methyl)- 4-(trifluoromethyl)isoindolin-1-one |
| 53 | 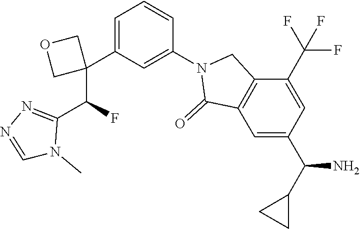 [SMILES: Cn1cnnc1[C@H](F)C1(c2cccc(N3Cc4c(cc([C@@H](N)C5CC5)cc4C(F)(F)F)C3=O)c2)COC1] | 6-((S)-amino(cyclopropyl)methyl)-2-(3- (3-((R)-fluoro(4-methyl-4H-1,2,4-triazol- 3-yl)methyl)oxetan-3-yl)phenyl)-4- (trifluoromethyl)isoindolin-1-one |
| 54 | 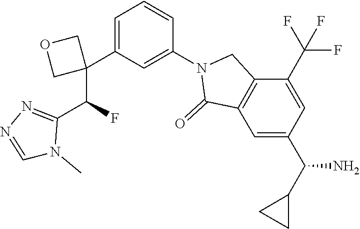 [SMILES: Cn1cnnc1[C@H](F)C1(c2cccc(N3Cc4c(cc([C@H](N)C5CC5)cc4C(F)(F)F)C3=O)c2)COC1] | 6-((R)-amino(cyclopropyl)methyl)-2-(3- (3-((R)-fluoro(4-methyl-4H-1,2,4-triazol- 3-yl)methyl)oxetan-3-yl)phenyl)-4- (trifluoromethyl)isoindolin-1-one |
| 55 | 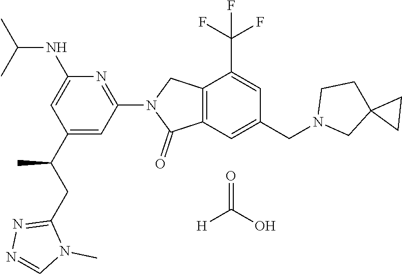 | (R)-6-(5-azaspiro[2.4]heptan-5- ylmethyl)-2-(6-(isopropylamino)-4-(1- (4-methyl-4H-1,2,4-triazol-3-yl)propan- 2-yl)pyridin-2-yl)-4- (trifluoromethyl)isoindolin-1-one formate |
| 56 | 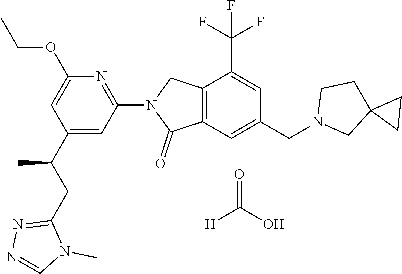 | (R)-6-(5-azaspiro[2.4]heptan-5- ylmethyl)-2-(6-ethoxy-4-(1-(4-methyl- 4H-1,2,4-triazol-3-yl)propan-2- yl)pyridin-2-yl)-4- (trifluoromethyl)isoindolin-1-one formate |
| 57 | 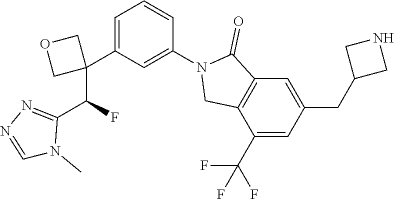 [SMILES: Cn1cnnc1[C@H](F)C1(c2cccc(N3Cc4c(cc(CC5CNC5)cc4C(F)(F)F)C3=O)c2)COC1] | ((R)-6-(azetidin-3-ylmethyl)-2-(3-(3- (fluoro(4-methyl-4H-1,2,4-triazol-3- yl)methyl)oxetan-3-yl)phenyl)-4- (trifluoromethyl)isoindolin-1-one |
| 58 | 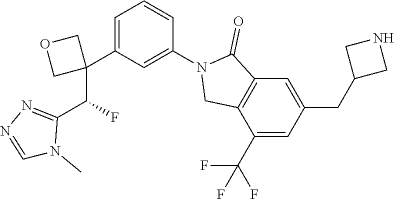 [SMILES: Cn1cnnc1[C@@H](F)C1(c2cccc(N3Cc4c(cc(CC5CNC5)cc4C(F)(F)F)C3=O)c2)COC1] | (S)-6-(azetidin-3-ylmethyl)-2-(3-(3- (fluoro(4-methyl-4H-1,2,4-triazol-3- yl)methyl)oxetan-3-yl)phenyl)-4- (trifluoromethyl)isoindolin-1-one |
| 59 | 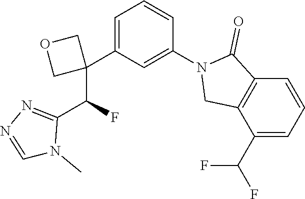 [SMILES: Cn1cnnc1[C@H](F)C1(c2cccc(N3Cc4c(cccc4C(F)F)C3=O)c2)COC1] | (R)-4-(difluoromethyl)-2-(3-(3-(fluoro(4- methyl-4H-1,2,4-triazol-3- yl)methyl)oxetan-3-yl)phenyl)isoindolin- 1-one |
| 66 | 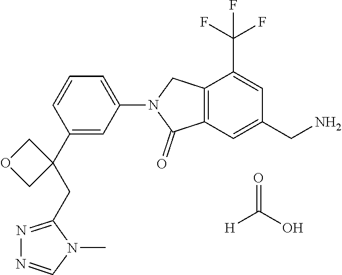 [SMILES: Cn1cnnc1CC1(c2cccc(N3Cc4c(cc(CN)cc4C(F)(F)F)C3=O)c2)COC1 ; O=CO] | 6-(aminomethyl)-2-(3-(3-((4-methyl-4H- 1,2,4-triazol-3-yl)methyl)oxetan-3- yl)phenyl)-4-(trifluoromethyl)isoindolin- 1-one formate |
| 68 | 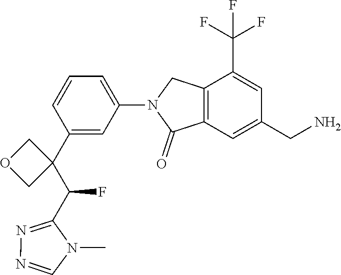 [SMILES: Cn1cnnc1[C@H](F)C1(c2cccc(N3Cc4c(cc(CN)cc4C(F)(F)F)C3=O)c2)COC1] | (R)-6-(aminomethyl)-2-(3-(3-(fluoro(4- methyl-4H-1,2,4-triazol-3- yl)methyl)oxetan-3-yl)phenyl)-4- (trifluoromethyl)isoindolin-1-one |
| 69 | 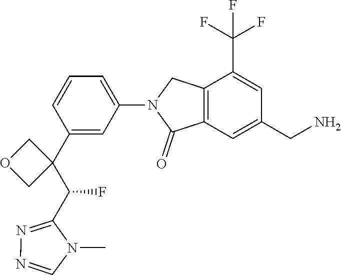 [SMILES: Cn1cnnc1[C@@H](F)C1(c2cccc(N3Cc4c(cc(CN)cc4C(F)(F)F)C3=O)c2)COC1] | (S)-6-(aminomethyl)-2-(3-(3-(fluoro(4- methyl-4H-1,2,4-triazol-3- yl)methyl)oxetan-3-yl)phenyl)-4- (trifluoromethyl)isoindolin-1-one |
| 70 | 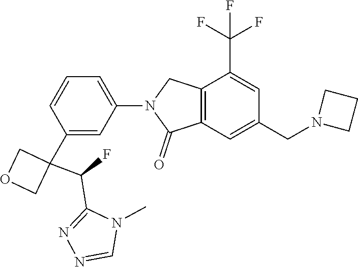 [SMILES: Cn1cnnc1[C@H](F)C1(c2cccc(N3Cc4c(cc(CN5CCC5)cc4C(F)(F)F)C3=O)c2)COC1] | (R)-6-(azetidin-1-ylmethyl)-2-(3-(3- (fluoro(4-methyl-4H-1,2,4-triazol-3- yl)methyl)oxetan-3-yl)phenyl)-4- (trifluoromethyl)isoindolin-1-one |
| 71 | 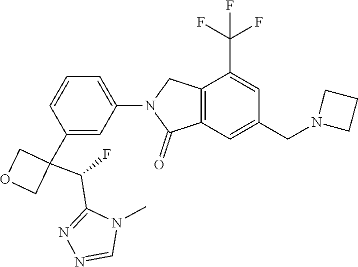 [SMILES: Cn1cnnc1[C@@H](F)C1(c2cccc(N3Cc4c(cc(CN5CCC5)cc4C(F)(F)F)C3=O)c2)COC1] | (S)-6-(azetidin-1-ylmethyl)-2-(3-(3- (fluoro(4-methyl-4H-1,2,4-triazol-3- yl)methyl)oxetan-3-yl)phenyl)-4- (trifluoromethyl)isoindolin-1-one |
| 72 | 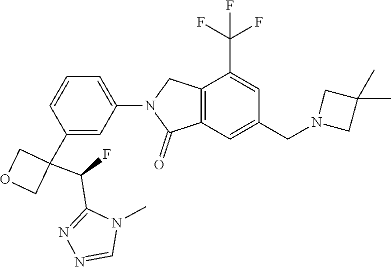 [SMILES: Cn1cnnc1[C@H](F)C1(c2cccc(N3Cc4c(cc(CN5CC(C)(C)C5)cc4C(F)(F)F)C3=O)c2)COC1] | (R)-6-((3,3-dimethylazetidin-1- yl)methyl)-2-(3-(3-(fluoro(4-methyl-4H- 1,2,4-triazol-3-yl)methyl)oxetan-3- yl)phenyl)-4-(trifluoromethyl)isoindolin- 1-one |
| 73 | 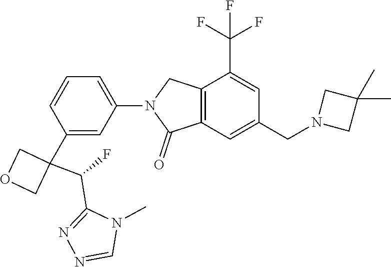 [SMILES: Cn1cnnc1[C@@H](F)C1(c2cccc(N3Cc4c(cc(CN5CC(C)(C)C5)cc4C(F)(F)F)C3=O)c2)COC1] | (S)-6-((3,3-dimethylazetidin-1- yl)methyl)-2-(3-(3-(fluoro(4-methyl-4H- 1,2,4-triazol-3-yl)methyl)oxetan-3- yl)phenyl)-4-(trifluoromethyl)isoindolin- 1-one |
| 74 | 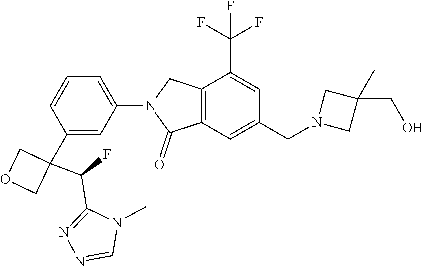 [SMILES: Cn1cnnc1[C@H](F)C1(c2cccc(N3Cc4c(cc(CN5CC(C)(CO)C5)cc4C(F)(F)F)C3=O)c2)COC1] | (R)-2-(3-(3-(fluoro(4-methyl-4H-1,2,4- triazol-3-yl)methyl)oxetan-3-yl)phenyl)- 6((3-(hydroxymethyl)-3- methylazetidin-1-yl)methyl)-4- (trifluoromethyl)isoindolin-1-one |
| 75 | 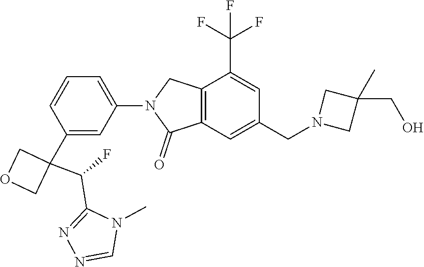 [SMILES: Cn1cnnc1[C@@H](F)C1(c2cccc(N3Cc4c(cc(CN5CC(C)(CO)C5)cc4C(F)(F)F)C3=O)c2)COC1] | (S)-2-(3-(3-(fluoro(4-methyl-4H-1,2,4- triazol-3-yl)methyl)oxetan-3-yl)phenyl)- 6((3-(hydroxymethyl)-3- methylazetidin-1-yl)methyl)-4- (trifluoromethyl)isoindolin-1-one |
| 76 | 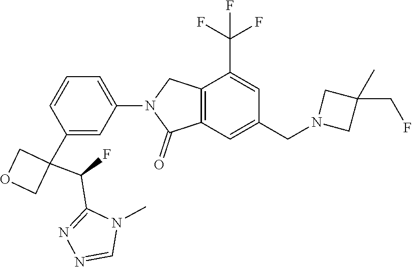 [SMILES: Cn1cnnc1[C@H](F)C1(c2cccc(N3Cc4c(cc(CN5CC(C)(CF)C5)cc4C(F)(F)F)C3=O)c2)COC1] | (R)-2-(3-(3-(fluoro(4-methyl-4H-1,2,4- triazol-3-yl)methyl)oxetan-3-yl)phenyl)- 6-((3-(fluoromethyl)-3-methylazetidin-1- yl)methyl)-4-(trifluoromethyl)isoindolin- 1-one |
| 77 | 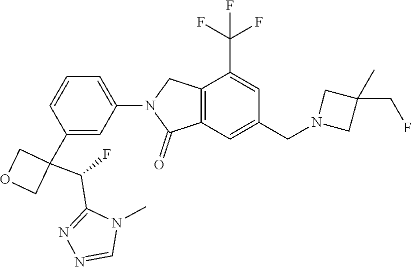 [SMILES: Cn1cnnc1[C@@H](F)C1(c2cccc(N3Cc4c(cc(CN5CC(C)(CF)C5)cc4C(F)(F)F)C3=O)c2)COC1] | (S)-2-(3-(3-(fluoro(4-methyl-4H-1,2,4- triazol-3-yl)methyl)oxetan-3-yl)phenyl)- 6-((3-(fluoromethyl)-3-methylazetidin-1- yl)methyl)-4-(trifluoromethyl)isoindolin- 1-one |
| 78 | 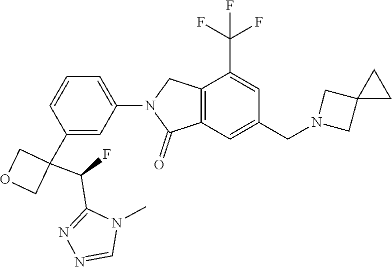 [SMILES: Cn1cnnc1[C@H](F)C1(c2cccc(N3Cc4c(cc(CN5CC6(CC6)C5)cc4C(F)(F)F)C3=O)c2)COC1] | (R)-6-((5-azaspiro[2.3]hexan-5- yl)methyl)-2-(3-(3-(fluoro(4-methyl-4H- 1,2,4-triazol-3-yl)methyl)oxetan-3- yl)phenyl)-4-(trifluoromethyl)isoindolin- 1-one |
| 79 | 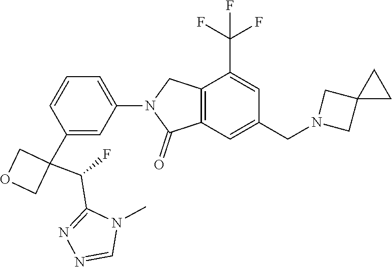 [SMILES: Cn1cnnc1[C@@H](F)C1(c2cccc(N3Cc4c(cc(CN5CC6(CC6)C5)cc4C(F)(F)F)C3=O)c2)COC1] | (S)-6-((5-azaspiro[2.3]hexan-5- yl)methyl)-2-(3-(3-(fluoro(4-methyl-4H- 1,2,4-triazol-3-yl)methypoxetan-3- yl)phenyl)-4-(trifluoromethyl)isoindolin- 1-one |
| 80 | 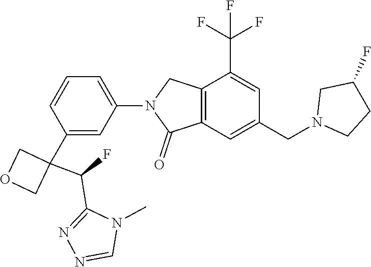 [SMILES: Cn1cnnc1[C@H](F)C1(c2cccc(N3Cc4c(cc(CN5CC[C@@H](F)C5)cc4C(F)(F)F)C3=O)c2)COC1] | 2-(3-(3-((R)-fluoro(4-methyl-4H-1,2,4- triazol-3-yl)methyl)oxetan-3-yl)phenyl)- 6-(((R)-3-fluoropyrrolidin-1-yl)methyl)- 4-(trifluoromethyl)isoindolin-1-one |
| 81 | 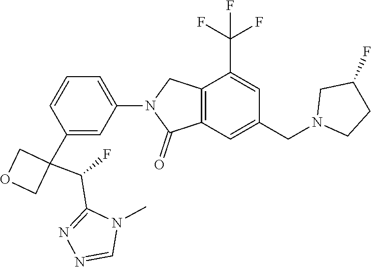 [SMILES: Cn1cnnc1[C@@H](F)C1(c2cccc(N3Cc4c(cc(CN5CC[C@@H](F)C5)cc4C(F)(F)F)C3=O)c2)COC1] | 2-(3-(3-((S)-fluoro(4-methyl-4H-1,2,4- triazol-3-yl)methyl)oxetan-3-yl)phenyl)- 6-(((R)-3-fluoropyrrolidin-1-yl)methyl)- 4-(trifluoromethyl)isoindolin-1-one |
| 82 | 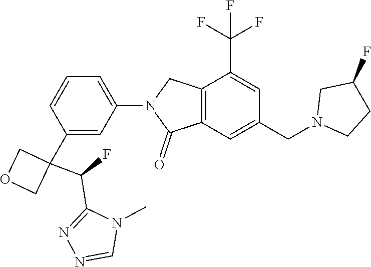 [SMILES: Cn1cnnc1[C@H](F)C1(c2cccc(N3Cc4c(cc(CN5CC[C@H](F)C5)cc4C(F)(F)F)C3=O)c2)COC1] | 2-(3-(3-((R)-fluoro(4-methyl-4H-1,2,4- triazol-3-yl)methyl)oxetan-3-yl)phenyl)- 6-(((S)-3-fluoropyrrolidin-1-yl)methyl)-4- (trifluoromethyl)isoindolin-1-one |
| 83 | 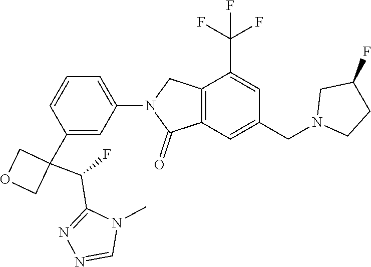 [SMILES: Cn1cnnc1[C@@H](F)C1(c2cccc(N3Cc4c(cc(CN5CC[C@H](F)C5)cc4C(F)(F)F)C3=O)c2)COC1] | 2-(3-(3-((S)-fluoro(4-methyl-4H-1,2,4- triazol-3-yl)methyl)oxetan-3-yl)phenyl)- 6-(((S)-3-fluoropyrrolidin-1-yl)methyl)-4- (trifluoromethyl)isoindolin-1-one |
| 84 | 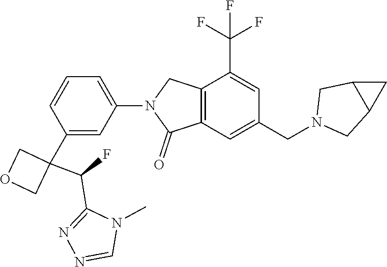 [SMILES: Cn1cnnc1[C@H](F)C1(c2cccc(N3Cc4c(cc(CN5CC6CC6C5)cc4C(F)(F)F)C3=O)c2)COC1] | 6-((3-azabicyclo[3.1.0]hexan-3- yl)methyl)-2-(3-(3-((R)-fluoro(4-methyl- 4H-1,2,4-triazol-3-yl)methyl)oxetan-3- yl)phenyl)-4-(trifluoromethyl)isoindolin- 1-one |
| 85 | 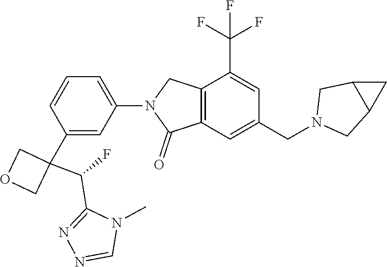 [SMILES: Cn1cnnc1[C@@H](F)C1(c2cccc(N3Cc4c(cc(CN5CC6CC6C5)cc4C(F)(F)F)C3=O)c2)COC1] | 6-((3-azabicyclo[3.1.0]hexan-3 yl)methyl)-2-(3-(3-((S)-fluoro(4-methyl- 4H-1,2,4-triazol-3-yl)methyl)oxetan-3- yl)phenyl)-4-(trifluoromethyl)isoindolin- 1-one |
| 86 | 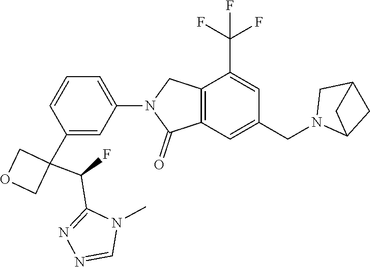 [SMILES: Cn1cnnc1[C@H](F)C1(c2cccc(N3Cc4c(cc(CN5CC6CC5C6)cc4C(F)(F)F)C3=O)c2)COC1] | (R)-6-((2-azabicyclo[2.1.1]hexan-2- yl)methyl)-2-(3-(3-(fluoro(4-methyl-4H- 1,2,4-triazol-3-yl)methyl)oxetan-3- yl)phenyl)-4-(trifluoromethyl)isoindolin- 1-one |
| 87 | 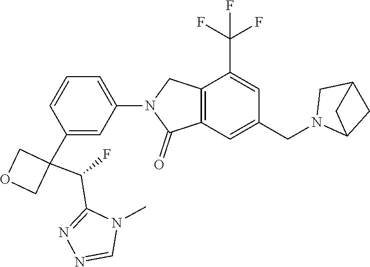 [SMILES: Cn1cnnc1[C@@H](F)C1(c2cccc(N3Cc4c(cc(CN5CC6CC5C6)cc4C(F)(F)F)C3=O)c2)COC1] | (S)-6-((2-azabicyclo[2.1.1]hexan-2- yl)methyl)-2-(3-(3-(fluoro(4-methyl-4H- 1,2,4-triazol-3-yl)methyl)oxetan-3- yl)phenyl)-4-(trifluoromethyl)isoindolin- 1-one |
| 88 | 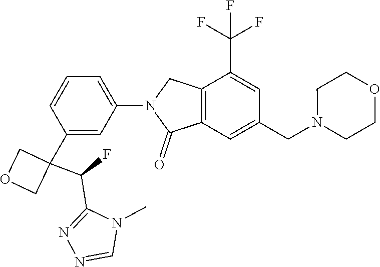 [SMILES: Cn1cnnc1[C@H](F)C1(c2cccc(N3Cc4c(cc(CN5CCOCC5)cc4C(F)(F)F)C3=O)c2)COC1] | (R)-2-(3-(3-(fluoro(4-methyl-4H-1,2,4- triazol-3-yl)methyl)oxetan-3-yl)phenyl)- 6-(morpholinomethyl)-4- (trifluoromethyl)isoindolin-1-one |
| 89 | 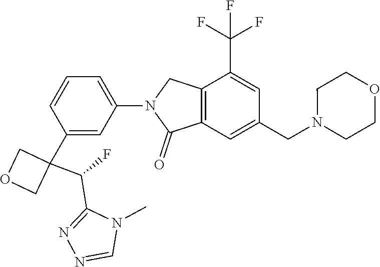 [SMILES: Cn1cnnc1[C@@H](F)C1(c2cccc(N3Cc4c(cc(CN5CCOCC5)cc4C(F)(F)F)C3=O)c2)COC1] | (S)-2-(3-(3-(fluoro(4-methyl-4H-1,2,4- triazol-3-yl)methyl)oxetan-3-yl)phenyl)- 6-(morpholinomethyl)-4- (trifluoromethyl)isoindolin-1-one |
| 90 | 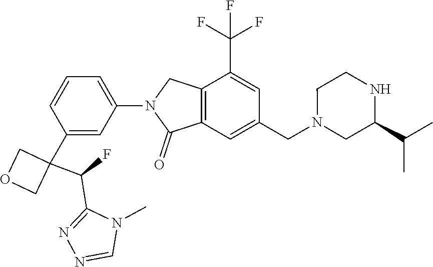 [SMILES: CC(C)[C@H]1CN(Cc2cc3c(c(C(F)(F)F)c2)CN(c2cccc(C4([C@@H](F)c5nncn5C)COC4)c2)C3=O)CCN1] | 2-(3-(3((R)-fluoro(4-methyl-4H-1,2,4- triazol-3-yl)methyl)oxetan-3-yl)phenyl)- 6-(((S)-3-isopropylpiperazin-1- yl)methyl)-4-(trifluoromethyl)isoindolin- 1-one |
| 91 | 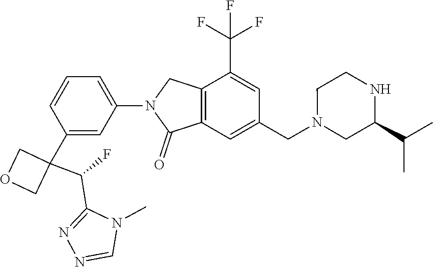 [SMILES: CC(C)[C@H]1CN(Cc2cc3c(c(C(F)(F)F)c2)CN(c2cccc(C4([C@H](F)c5nncn5C)COC4)c2)C3=O)CCN1] | 2-(3-(3-((S)-fluoro(4-methyl-4H-1,2,4- triazol-3-yl)methyl)oxetan-3-yl)phenyl)- 6-(((S)-3-isopropylpiperazin-1- yl)methyl-4-(trifluoromethyl)isoindolin- 1-one |
| 92 | 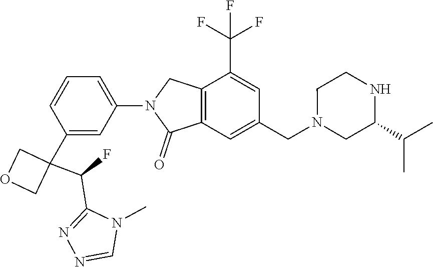 [SMILES: CC(C)[C@@H]1CN(Cc2cc3c(c(C(F)(F)F)c2)CN(c2cccc(C4([C@@H](F)c5nncn5C)COC4)c2)C3=O)CCN1] | 2-(3-(34(R)-((R)-methyl-4H-1,2,4- triazol-3-yl)methyl)oxetan-3-yl)phenyl)- 6-(((R)-3-isopropylpiperazin-1- yl)methyl)-4-(trifluoromethyl)isoindolin- 1-one |
| 93 | 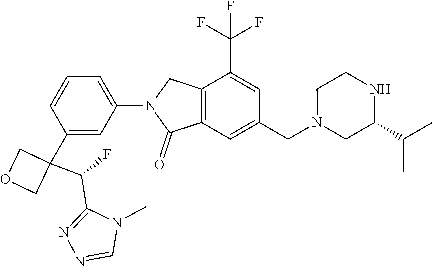 [SMILES: CC(C)[C@@H]1CN(Cc2cc3c(c(C(F)(F)F)c2)CN(c2cccc(C4([C@H](F)c5nncn5C)COC4)c2)C3=O)CCN1] | 2-(3-(3-((S)-fluoro(4-methyl-4H-1,2,4- triazol-3-yl)methyl)oxetan-3-yl)phenyl)- 6-(((R)-3-isopropylpiperazin-1- yl)methyl)-4-(trifluoromethyl)isoindolin- 1-one |
| 94 | 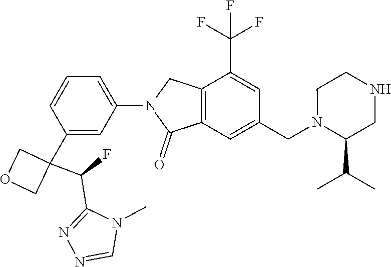 [SMILES: CC(C)[C@@H]1CNCCN1Cc1cc2c(c(C(F)(F)F)c1)CN(c1cccc(C3([C@@H](F)c4nncn4C)COC3)c1)C2=O] | 2-(3-(3((R)-fluoro(4-methyl-4H-1,2,4- triazol-3-yl)methyl)oxetan-3-yl)phenyl)- 6-(((R)-2-isopropylpiperazin-1- yl)methyl)-4-(trifluoromethyl)isoindolin- 1-one |
| 95 | 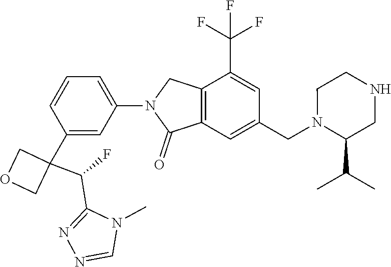 [SMILES: CC(C)[C@@H]1CNCCN1Cc1cc2c(c(C(F)(F)F)c1)CN(c1cccc(C3([C@H](F)c4nncn4C)COC3)c1)C2=O] | 2-(3-(3-((S)-fluoro(4-methyl-4H-1,2,4- triazol-3-yl)methyl)oxetan-3-yl)phenyl)- 6-(((R)-2-isopropylpiperazin-1- yl)methyl)-4-(trifluoromethyl)isoindolin- 1-one |
| 96 | 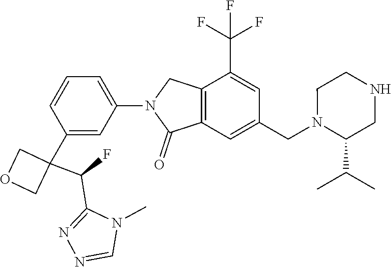 [SMILES: CC(C)[C@H]1CNCCN1Cc1cc2c(c(C(F)(F)F)c1)CN(c1cccc(C3([C@@H](F)c4nncn4C)COC3)c1)C2=O] | 2-(3-(3((R)-fluoro(4-methyl-4H-1,2,4- triazol-3-yl)methyl)oxetan-3-yl)phenyl)- 6-(((S)-2-isopropylpiperazin-1- yl)methyl)-4-(trifluoromethyl)isoindolin- 1-one |
| 97 | 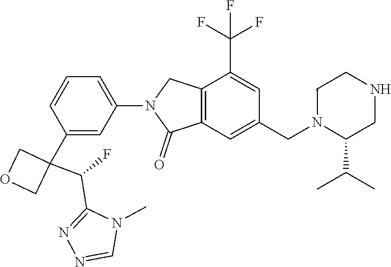 [SMILES: CC(C)[C@H]1CNCCN1Cc1cc2c(c(C(F)(F)F)c1)CN(c1cccc(C3([C@H](F)c4nncn4C)COC3)c1)C2=O] | 2-(3-(3-((S)-fluoro(4-methyl-4H-1,2,4- triazol-3-yl)methyl)oxetan-3-yl)phenyl)- 6-(((S)-2-isopropylpiperazin-1- yl)methyl)-4-(trifluoromethyl)isoindolin- 1-one |
| 99 | 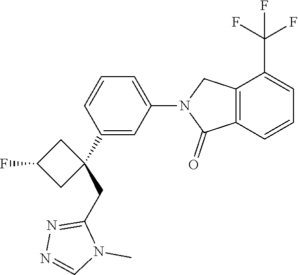 [SMILES: Cn1cnnc1C[C@]1(c2cccc(N3Cc4c(cccc4C(F)(F)F)C3=O)c2)C[C@@H](F)C1] | 2-(3-((1s,3r)-3-fluoro-1-((4-methyl-4H- 1,2,4-triazol-3- yl)methyl)cyclobutyl)phenyl)-4- (trifluoromethyl)isoindolin-1-one |
| 100 | 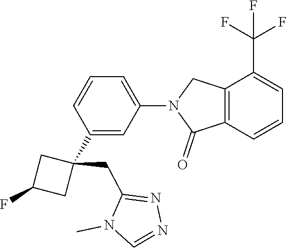 [SMILES: Cn1cnnc1C[C@]1(c2cccc(N3Cc4c(cccc4C(F)(F)F)C3=O)c2)C[C@H](F)C1] | 2-(3-((1r,3s)-3-fluoro-1-((4-methyl-4H- 1,2,4-triazol-3- yl)methyl)cyclobutyl)phenyl)-4- (trifluoromethyl)isoindolin-1-one |
| 101 | 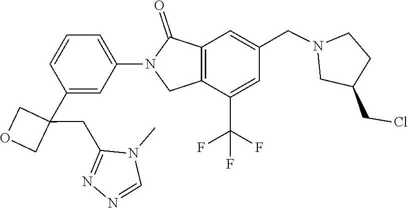 [SMILES: Cn1cnnc1CC1(c2cccc(N3Cc4c(cc(CN5CC[C@@H](CCl)C5)cc4C(F)(F)F)C3=O)c2)COC1] | (R)-6-((3-(chloromethyl)pyrrolidin-1- yl)methyl)-2-(3-(3-((4-methyl-4H-1,2,4- triazol-3-yl)methyl)oxetan-3-yl)phenyl)- 4-(trifluoromethyl)isoindolin-1-one |
| 102 | 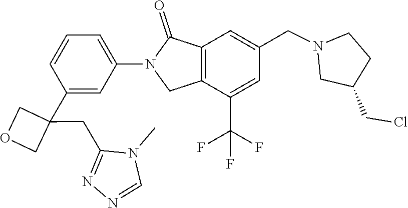 [SMILES: Cn1cnnc1CC1(c2cccc(N3Cc4c(cc(CN5CC[C@H](CCl)C5)cc4C(F)(F)F)C3=O)c2)COC1] | (S)-6-((3-(chloromethyl)pyrrolidin-1- yl)methyl)-2-(3-(3-((4-methyl-4H-1,2,4- triazol-3-yl)methyl)oxetan-3-yl)phenyl)- 4-(trifluoromethyl)isoindolin-1-one |
| 103 | 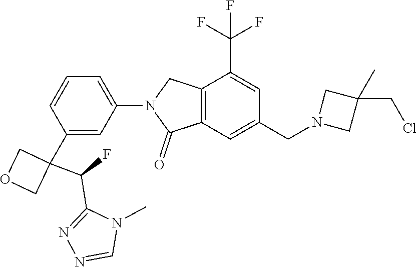 [SMILES: Cn1cnnc1[C@H](F)C1(c2cccc(N3Cc4c(cc(CN5CC(C)(CCl)C5)cc4C(F)(F)F)C3=O)c2)COC1] | (R)-6-((3-(chloromethyl)-3- methylazetidin-1-yl)methyl)-2-(3-(3- (fluoro(4-methyl-4H-1,2,4-triazol-3- yl)methyl)oxetan-3-yl)phenyl)-4- (trifluoromethyl)isoindolin-1-one |
| 104 | 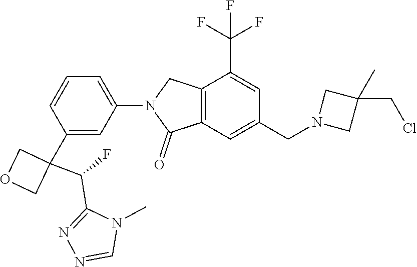 [SMILES: Cn1cnnc1[C@@H](F)C1(c2cccc(N3Cc4c(cc(CN5CC(C)(CCl)C5)cc4C(F)(F)F)C3=O)c2)COC1] | (S)-6-((3-(chloromethyl)-3- methylazetidin-1-yl)methyl)-2-(3-(3- (fluoro(4-methyl-4H-1,2,4-triazol-3- yl)methyl)oxetan-3-yl)phenyl)-4- (trifluoromethyl)isoindolin-1-one |
| 105 | 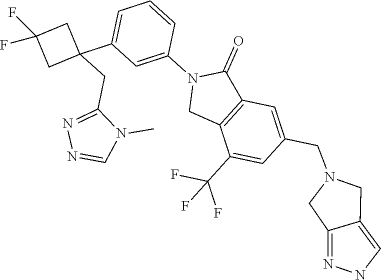 [SMILES: Cn1cnnc1CC1(c2cccc(N3Cc4c(cc(CN5Cc6c[nH]nc6C5)cc4C(F)(F)F)C3=O)c2)CC(F)(F)C1] | 2-(3-(3,3-difluoro-1-((4-methyl-4H- 1,2,4-triazol-3- yl)methyl)cyclobutyl)phenyl)-6-((2,6- dihydropyrrolo[3,4-c]pyrazol-5(4H)- yl)methyl)-4-(trifluoromethyl)isoindolin- 1-one |
| 106 | 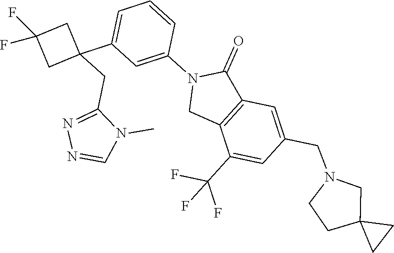 [SMILES: Cn1cnnc1CC1(c2cccc(N3Cc4c(cc(CN5CCC6(CC6)C5)cc4C(F)(F)F)C3=O)c2)CC(F)(F)C1] | 6((5-azaspiro[2.4]heptan-5-yl)methyl)- 2-(3-(3,3-difluoro-1-((4-methyl-4H- 1,2,4-triazol-3- yl)methyl)cyclobutyl)phenyl)-4- (trifluoromethyl)isoindolin-1-one |
| 107 | 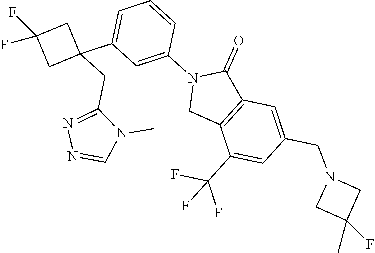 [SMILES: Cn1cnnc1CC1(c2cccc(N3Cc4c(cc(CN5CC(C)(F)C5)cc4C(F)(F)F)C3=O)c2)CC(F)(F)C1] | 2-(3-(3,3-difluoro-1-((4-methyl-4H- 1,2,4-triazol-3- yl)methyl)cyclobutyl)phenyl)-6-((3- fluoro-3-methylazetidin-1-yl)methyl)-4- (trifluoromethyl)isoindolin-1-one |
| 108 | 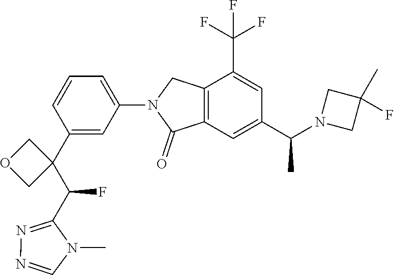 [SMILES: C[C@@H](c1cc2c(c(C(F)(F)F)c1)CN(c1cccc(C3([C@@H](F)c4nncn4C)COC3)c1)C2=O)N1CC(C)(F)C1] | 2-(3-(3((R)-fluoro(4-methyl-4H-1,2,4- triazol-3-yl)methyl)oxetan-3-yl)phenyl)- 6-((S)-1-(3-fluoro-3-methylazetidin-1- yl)ethyl)-4-(trifluoromethyl)isoindolin-1- one |
| 109 | 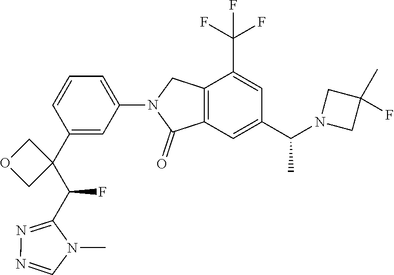 [SMILES: C[C@H](c1cc2c(c(C(F)(F)F)c1)CN(c1cccc(C3([C@@H](F)c4nncn4C)COC3)c1)C2=O)N1CC(C)(F)C1] | 2-(3-(3((R)-fluoro(4-methyl-4H-1,2,4- triazol-3-yl)methyl)oxetan-3-yl)phenyl)- 6-((R)-1-(3-fluoro-3-methylazetidin-1- yl)ethyl)-4-(trifluoromethyl)isoindolin-1- one |
| 110 | 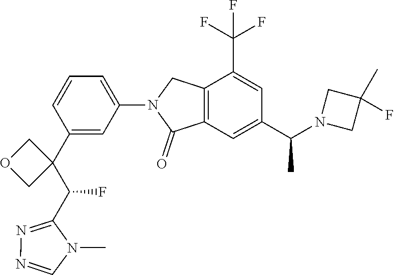 [SMILES: C[C@@H](c1cc2c(c(C(F)(F)F)c1)CN(c1cccc(C3([C@H](F)c4nncn4C)COC3)c1)C2=O)N1CC(C)(F)C1] | 2-(3-(3-((S)-fluoro(4-methyl-4H-1,2,4- triazol-3-yl)methyl)oxetan-3-yl)phenyl)- 6-((S)-1-(3-fluoro-3-methylazetidin-1- yl)ethyl)-4-(trifluoromethyl)isoindolin-1- one |
| 111 | 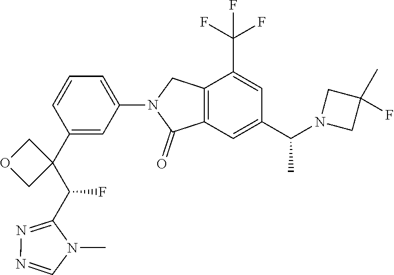 [SMILES: C[C@H](c1cc2c(c(C(F)(F)F)c1)CN(c1cccc(C3([C@H](F)c4nncn4C)COC3)c1)C2=O)N1CC(C)(F)C1] | 2-(3-(3-((S)-fluoro(4-methyl-4H-1,2,4- triazol-3-yl)methyl)oxetan-3-yl)phenyl)- 6-((R)-1-(3-fluoro-3-methylazetidin-1- yl)ethyl)-4-(trifluoromethyl)isoindolin-1- one |
| 116 | 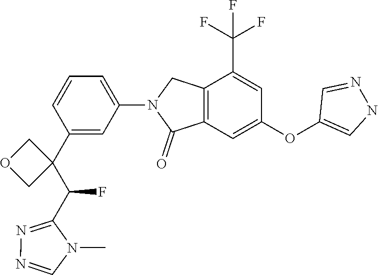 [SMILES: Cn1cnnc1[C@H](F)C1(c2cccc(N3Cc4c(cc(Oc5cn[nH]c5)cc4C(F)(F)F)C3=O)c2)COC1] | (R)-6-((1H-pyrazol-4-yl)oxy)-2-(3-(3- (fluoro(4-methyl-4H-1,2,4-triazol-3- yl)methyl)oxetan-3-yl)phenyl)-4- (trifluoromethyl)isoindolin-1-one |
| 117 | 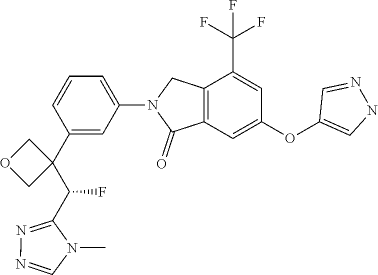 [SMILES: Cn1cnnc1[C@@H](F)C1(c2cccc(N3Cc4c(cc(Oc5cn[nH]c5)cc4C(F)(F)F)C3=O)c2)COC1] | (S)-6-((1H-pyrazol-4-yl)oxy)-2-(3-(3- (fluoro(4-methyl-4H-1,2,4-triazol-3- yl)methyl)oxetan-3-yl)phenyl)-4- (trifluoromethyl)isoindolin-1-one |
| 118 | 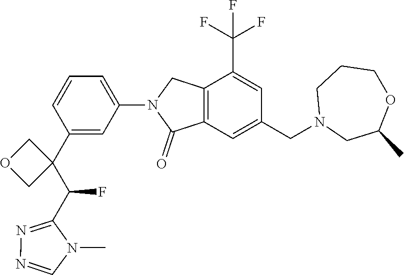 [SMILES: C[C@H]1CN(Cc2cc3c(c(C(F)(F)F)c2)CN(c2cccc(C4([C@@H](F)c5nncn5C)COC4)c2)C3=O)CCCO1] | 2-(3-(3-((R)-fluoro(4-methyl-4H-1,2,4- triazol-3-yl)methyl)oxetan-3-yl)phenyl)- 6-(((S)-2-methyl-1,4-oxazepan-4- yl)methyl)-4-(trifluoromethyl)isoindolin- 1-one |
| 119 | 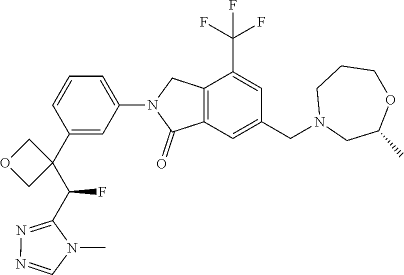 [SMILES: C[C@@H]1CN(Cc2cc3c(c(C(F)(F)F)c2)CN(c2cccc(C4([C@@H](F)c5nncn5C)COC4)c2)C3=O)CCCO1] | 2-(3-(3((R)-fluoro(4-methyl-4H-1,2,4- triazol-3-yl)methyl)oxetan-3-yl)phenyl)- 6-(((R)-2-methyl-1,4-oxazepan-4- yl)methyl)-4-(trifluoromethyl)isoindolin- 1-one |
| 120 | 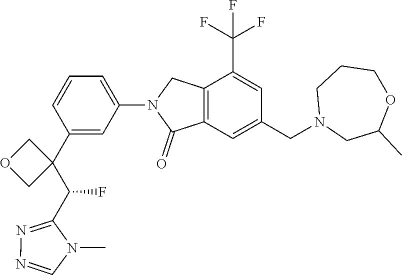 [SMILES: CC1CN(Cc2cc3c(c(C(F)(F)F)c2)CN(c2cccc(C4([C@H](F)c5nncn5C)COC4)c2)C3=O)CCCO1] | 2-(3-(3-((S)-fluoro(4-methyl-4H-1,2,4- triazol-3-yl)methyl)oxetan-3-yl)phenyl)- 6-((2-methyl-1,4-oxazepan-4- yl)methyl)-4-(trifluoromethyl)isoindolin- 1-one |
| 125 | 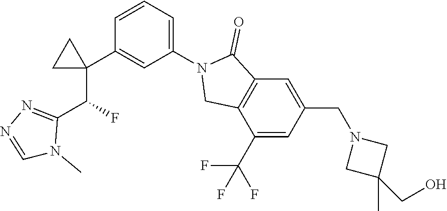 [SMILES: Cn1cnnc1[C@@H](F)C1(c2cccc(N3Cc4c(cc(CN5CC(C)(CO)C5)cc4C(F)(F)F)C3=O)c2)CC1] | (S)-2-(3-(1-(fluoro(4-methyl-4H-1,2,4- triazol-3-yl)methyl)cyclopropyl)phenyl)- 6((3-(hydroxymethyl)-3- methylazetidin-1-yl)methyl)-4- (trifluoromethyl)isoindolin-1-one |
| 126 | 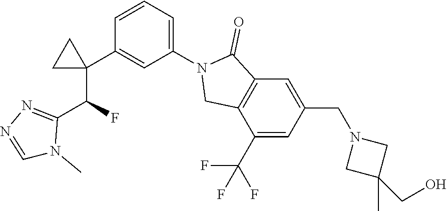 [SMILES: Cn1cnnc1[C@H](F)C1(c2cccc(N3Cc4c(cc(CN5CC(C)(CO)C5)cc4C(F)(F)F)C3=O)c2)CC1] | (R)-2-(3-(1-(fluoro(4-methyl-4H-1,2,4- triazol-3-yl)methyl)cyclopropyl)phenyl)- 6((3-(hydroxymethyl)-3- methylazetidin-1-yl)methyl)-4- (trifluoromethyl)isoindolin-1-one |
| 127 | 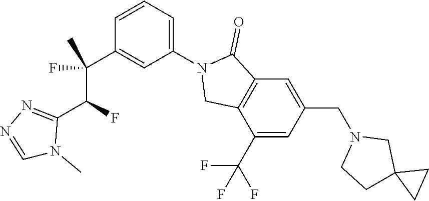 [SMILES: Cn1cnnc1[C@H](F)[C@](C)(F)c1cccc(N2Cc3c(cc(CN4CCC5(CC5)C4)cc3C(F)(F)F)C2=O)c1] | 6-(5-azaspiro[2.4]heptan-5-ylmethyl)-2- (3-((1S,2R)-1,2-difluoro-1-(4-methyl- 4H-1,2,4-triazol-3-yl)propan-2- yl)phenyl)-4-(trifluoromethyl)isoindolin- 1-one |
| 128 | 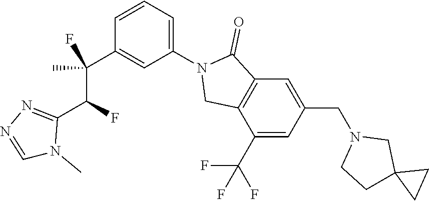 [SMILES: Cn1cnnc1[C@H](F)[C@@](C)(F)c1cccc(N2Cc3c(cc(CN4CCC5(CC5)C4)cc3C(F)(F)F)C2=O)c1] | 6-(5-azaspiro[2.4]heptan-5-ylmethyl)-2- (3-((1S,2S)-1,2-difluoro-1-(4-methyl- 4H-1,2,4-triazol-3-yl)propan-2- yl)phenyl)-4-(trifluoromethyl)isoindolin- 1-one |
| 129 | 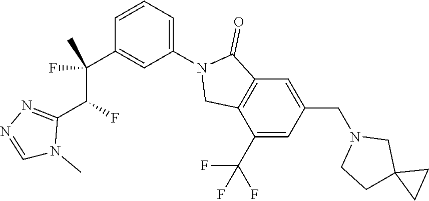 [SMILES: Cn1cnnc1[C@@H](F)[C@](C)(F)c1cccc(N2Cc3c(cc(CN4CCC5(CC5)C4)cc3C(F)(F)F)C2=O)c1] | 6-(5-azaspiro[2.4]heptan-5-ylmethyl)-2- (3-((1R,2R)-1,2-difluoro-1-(4-methyl- 4H-1,2,4-triazol-3-yl)propan-2- yl)phenyl)-4-(trifluoromethyl)isoindolin- 1-one |
| 130 | 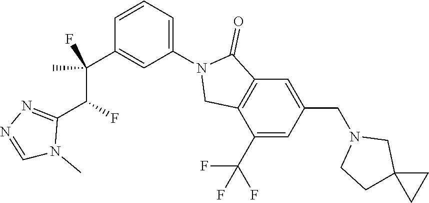 [SMILES: Cn1cnnc1[C@@H](F)[C@@](C)(F)c1cccc(N2Cc3c(cc(CN4CCC5(CC5)C4)cc3C(F)(F)F)C2=O)c1] | 6-(5-azaspiro[2.4]heptan-5-ylmethyl)-2- (3-((1R,2S)-1,2-difluoro-1-(4-methyl- 4H-1,2,4-triazol-3-yl)propan-2- yl)phenyl)-4-(trifluoromethyl)isoindolin- 1-one |
| 131 | 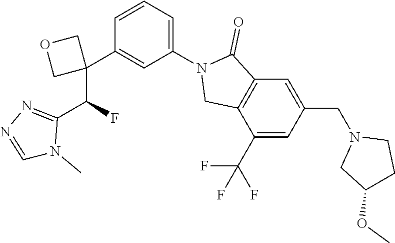 [SMILES: CO[C@H]1CCN(Cc2cc3c(c(C(F)(F)F)c2)CN(c2cccc(C4([C@@H](F)c5nncn5C)COC4)c2)C3=O)C1] | 2-(3-(3-((R)-fluoro(4-methyl-4H-1,2,4- triazol-3-yl)methyl)oxetan-3-yl)phenyl)- 6-(((S)-3-methoxypyrrolidin-1- yl)methyl)-4-(trifluoromethyl)isoindolin- 1-one |
| 132 | 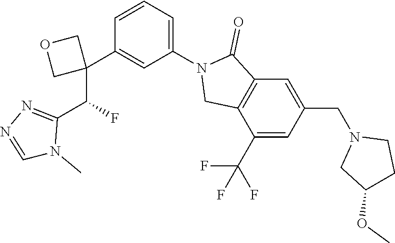 [SMILES: CO[C@H]1CCN(Cc2cc3c(c(C(F)(F)F)c2)CN(c2cccc(C4([C@H](F)c5nncn5C)COC4)c2)C3=O)C1] | 2-(3-(3-((S)-fluoro(4-methyl-4H-1,2,4- triazol-3-yl)methyl)oxetan-3-yl)phenyl)- 6-(((S)-3-methoxypyrrolidin-1- yl)methyl)-4-(trifluoromethyl)isoindolin- 1-one |
| 133 | 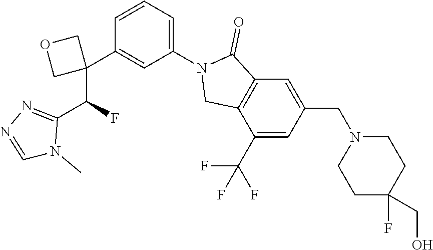 [SMILES: Cn1cnnc1[C@H](F)C1(c2cccc(N3Cc4c(cc(CN5CCC(F)(CO)CC5)cc4C(F)(F)F)C3=O)c2)COC1] | (R)-2-(3-(3-(fluoro(4-methyl-4H-1,2,4- triazol-3-yl)methyl)oxetan-3-yl)phenyl)- 6-((4-fluoro-4-(hydroxymethyl)piperidin- 1-yl)methyl)-4- (trifluoromethyl)isoindolin-1-one |
| 134 | 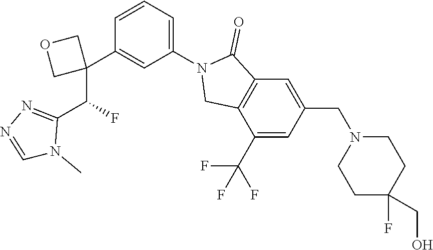 [SMILES: Cn1cnnc1[C@@H](F)C1(c2cccc(N3Cc4c(cc(CN5CCC(F)(CO)CC5)cc4C(F)(F)F)C3=O)c2)COC1] | (S)-2-(3-(3-(fluoro(4-methyl-4H-1,2,4- triazol-3-yl)methyl)oxetan-3-yl)phenyl)- 6-((4-fluoro-4-(hydroxymethyl)piperidin- 1-yl)methyl)-4- (trifluoromethyl)isoindolin-1-one |
| 135 | 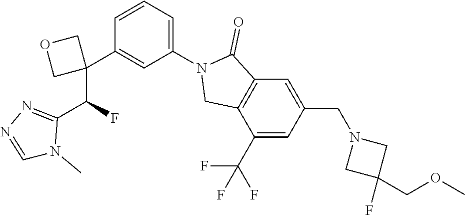 [SMILES: COCC1(F)CN(Cc2cc3c(c(C(F)(F)F)c2)CN(c2cccc(C4([C@@H](F)c5nncn5C)COC4)c2)C3=O)C1] | (R)-2-(3-(3-(fluoro(4-methyl-4H-1,2,4- triazol-3-yl)methyl)oxetan-3-yl)phenyl)- 6-((3-fluoro-3-(methoxymethyl)azetidin- 1-yl)methyl)-4- (trifluoromethyl)isoindolin-1-one |
| 136 | 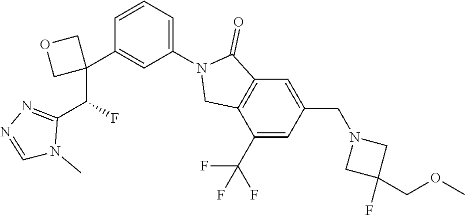 [SMILES: COCC1(F)CN(Cc2cc3c(c(C(F)(F)F)c2)CN(c2cccc(C4([C@H](F)c5nncn5C)COC4)c2)C3=O)C1] | (S)-2-(3-(3-(fluoro(4-methyl-4H-1,2,4- triazol-3-yl)methyl)oxetan-3-yl)phenyl)- 6-((3-fluoro-3-(methoxymethyl)azetidin- 1-yl)methyl)-4- (trifluoromethyl)isoindolin-1-one |
| 137 | 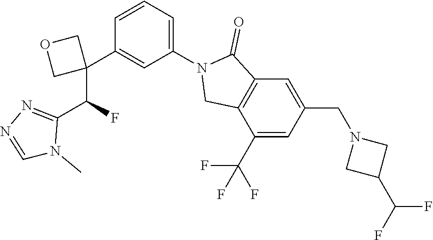 [SMILES: Cn1cnnc1[C@H](F)C1(c2cccc(N3Cc4c(cc(CN5CC(C(F)F)C5)cc4C(F)(F)F)C3=O)c2)COC1] | (R)-6-((3-(difluoromethyl)azetidin-1- yl)methyl)-2-(3-(3-(fluoro(4-methyl-4H- 1,2,4-triazol-3-yl)methyl)oxetan-3- yl)phenyl)-4-(trifluoromethyl)isoindolin- 1-one |
| 138 | 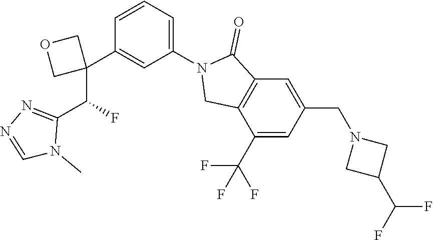 [SMILES: Cn1cnnc1[C@@H](F)C1(c2cccc(N3Cc4c(cc(CN5CC(C(F)F)C5)cc4C(F)(F)F)C3=O)c2)COC1] | (S)-6-((3-(difluoromethyl)azetidin-1- yl)methyl)-2-(3-(3-(fluoro(4-methyl-4H- 1,2,4-triazol-3-yl)methyl)oxetan-3- yl)phenyl)-4-(trifluoromethyl)isoindolin- 1-one |
| 139 | 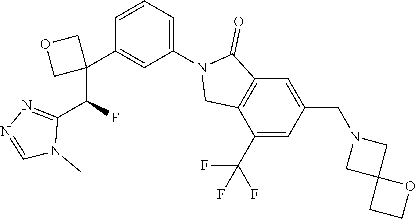 [SMILES: Cn1cnnc1[C@H](F)C1(c2cccc(N3Cc4c(cc(CN5CC6(CCO6)C5)cc4C(F)(F)F)C3=O)c2)COC1] | (R)-6-(1-oxa-6-azaspiro[3.3]heptan-6- ylmethyl)-2-(3-(3-(fluoro(4-methyl-4H- 1,2,4-triazol-3-yl)methyl)oxetan-3- yl)phenyl)-4-(trifluoromethyl)isoindolin- 1-one |
| 140 | 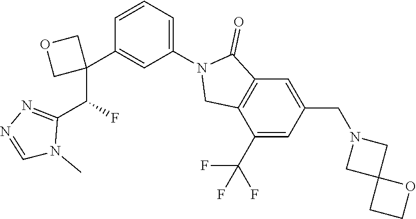 [SMILES: Cn1cnnc1[C@@H](F)C1(c2cccc(N3Cc4c(cc(CN5CC6(CCO6)C5)cc4C(F)(F)F)C3=O)c2)COC1] | (S)-6-(1-oxa-6-azaspiro[3.3]heptan-6- ylmethyl)-2-(3-(3-(fluoro(4-methyl-4H- 1,2,4-triazol-3-yl)methyl)oxetan-3- yl)phenyl)-4-(trifluoromethyl)isoindolin- 1-one |
| 141 | 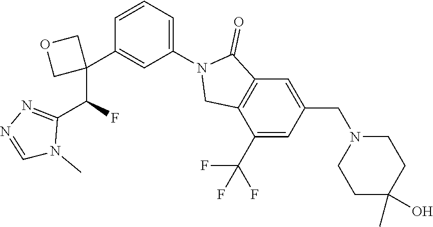 [SMILES: Cn1cnnc1[C@H](F)C1(c2cccc(N3Cc4c(cc(CN5CCC(C)(O)CC5)cc4C(F)(F)F)C3=O)c2)COC1] | (R)-2-(3-(3-(fluoro(4-methyl-4H-1,2,4- triazol-3-yl)methyl)oxetan-3-yl)phenyl)- 6-((4-hydroxy-4-methylpiperidin-1- yl)methyl)-4-(trifluoromethyl)isoindolin- 1-one |
| 142 | 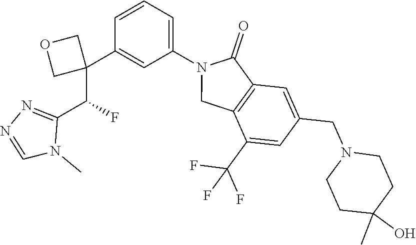 [SMILES: Cn1cnnc1[C@@H](F)C1(c2cccc(N3Cc4c(cc(CN5CCC(C)(O)CC5)cc4C(F)(F)F)C3=O)c2)COC1] | (S)-2-(3-(3-(fluoro(4-methyl-4H-1,2,4- triazol-3-yl)methyl)oxetan-3-yl)phenyl)- 6-((4-hydroxy-4-methylpiperidin-1- yl)methyl)-4-(trifluoromethyl)isoindolin- 1-one |
| 143 | 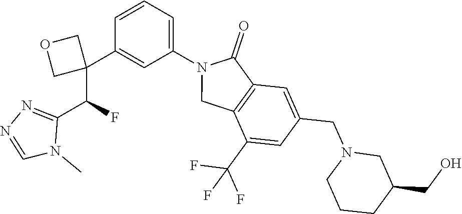 [SMILES: Cn1cnnc1[C@H](F)C1(c2cccc(N3Cc4c(cc(CN5CCC[C@H](CO)C5)cc4C(F)(F)F)C3=O)c2)COC1] | 2-(3-(3-((R)-fluoro(4-methyl-4H-1,2,4- triazol-3-yl)methyl)oxetan-3-yl)phenyl)- 6-(((S)-3-(hydroxymethyl)piperidin-1- yl)methyl)-4-(trifluoromethyl)isoindolin- 1-one |
| 144 | 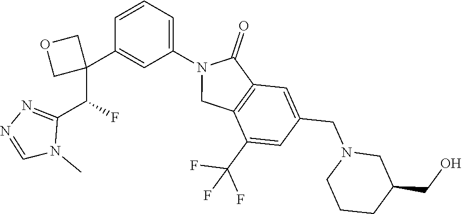 [SMILES: Cn1cnnc1[C@@H](F)C1(c2cccc(N3Cc4c(cc(CN5CCC[C@H](CO)C5)cc4C(F)(F)F)C3=O)c2)COC1] | 2-(3-(3-((S)-fluoro(4-methyl-4H-1,2,4- triazol-3-yl)methyl)oxetan-3-yl)phenyl)- 6-(((S)-3-(hydroxymethyl)piperidin-1- yl)methyl)-4-(trifluoromethyl)isoindolin- 1-one |
| 145 | 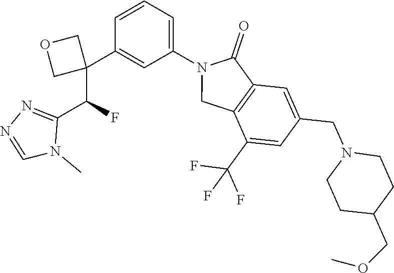 [SMILES: COCC1CCN(Cc2cc3c(c(C(F)(F)F)c2)CN(c2cccc(C4([C@@H](F)c5nncn5C)COC4)c2)C3=O)CC1] | (R)-2-(3-(3-(fluoro(4-methyl-4H-1,2,4- triazol-3-yl)methyl)oxetan-3-yl)phenyl)- 6-((4-(methoxymethyl)piperidin-1- yl)methyl)-4-(trifluoromethyl)isoindolin- 1-one |
| 146 | 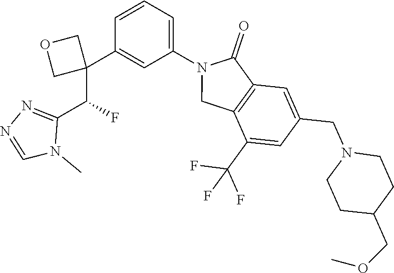 [SMILES: COCC1CCN(Cc2cc3c(c(C(F)(F)F)c2)CN(c2cccc(C4([C@H](F)c5nncn5C)COC4)c2)C3=O)CC1] | (S)-2-(3-(3-(fluoro(4-methyl-4H-1,2,4- triazol-3-yl)methyl)oxetan-3-yl)phenyl)- 6-((4-(methoxymethyl)piperidin-1- yl)methyl)-4-(trifluoromethyl)isoindolin- 1-one |
| 147 | 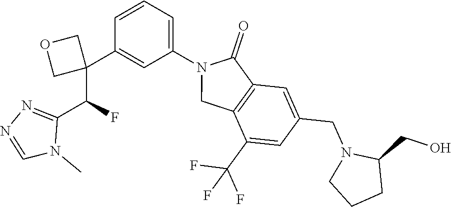 [SMILES: Cn1cnnc1[C@H](F)C1(c2cccc(N3Cc4c(cc(CN5CCC[C@@H]5CO)cc4C(F)(F)F)C3=O)c2)COC1] | 2-(3-(3-((R)-fluoro(4-methyl-4H-1,2,4- triazol-3-yl)methyl)oxetan-3-yl)phenyl)- 6-(((R)-2-(hydroxymethyl)pyrrolidin-1- yl)methyl)-4-(trifluoromethyl)isoindolin- 1-one |
| 148 | 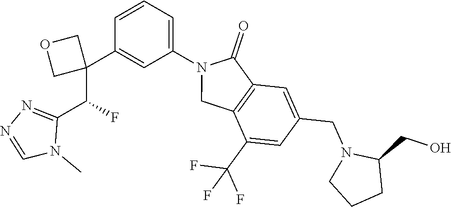 [SMILES: Cn1cnnc1[C@@H](F)C1(c2cccc(N3Cc4c(cc(CN5CCC[C@@H]5CO)cc4C(F)(F)F)C3=O)c2)COC1] | 2-(3-(3-((S)-fluoro(4-methyl-4H-1,2,4- triazol-3-yl)methyl)oxetan-3-yl)phenyl)- 6-(((R)-2-(hydroxymethyl)pyrrolidin-1- yl)methyl)-4-(trifluoromethyl)isoindolin- 1-one |
| 149 | 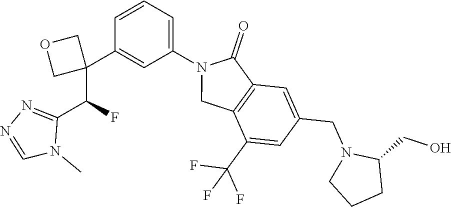 [SMILES: Cn1cnnc1[C@H](F)C1(c2cccc(N3Cc4c(cc(CN5CCC[C@H]5CO)cc4C(F)(F)F)C3=O)c2)COC1] | 2-(3-(3-((R)-fluoro(4-methyl-4H-1,2,4- triazol-3-yl)methyl)oxetan-3-yl)phenyl)- 6-(((S)-2-(hydroxymethyl)pyrrolidin-1- yl)methyl)-4-(trifluoromethyl)isoindolin- 1-one |
| 150 | 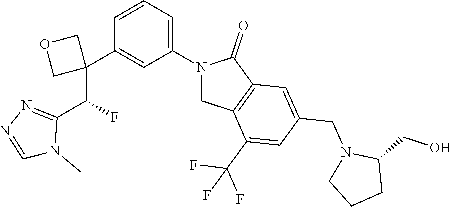 [SMILES: Cn1cnnc1[C@@H](F)C1(c2cccc(N3Cc4c(cc(CN5CCC[C@H]5CO)cc4C(F)(F)F)C3=O)c2)COC1] | 2-(3-(3-((S)-fluoro(4-methyl-4H-1,2,4- triazol-3-yl)methyl)oxetan-3-yl)phenyl)- 6-(((S)-2-(hydroxymethyl)pyrrolidin-1- yl)methyl)-4-(trifluoromethyl)isoindolin- 1-one |
| 151 | 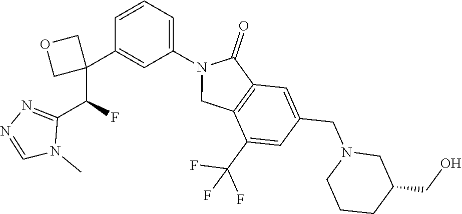 [SMILES: Cn1cnnc1[C@H](F)C1(c2cccc(N3Cc4c(cc(CN5CCC[C@@H](CO)C5)cc4C(F)(F)F)C3=O)c2)COC1] | 2-(3-(3-((R)-fluoro(4-methyl-4H-1,2,4- triazol-3-yl)methyl)oxetan-3-yl)phenyl)- 6-(((R)-3-(hydroxymethyl)piperidin-1- yl)methyl)-4-(trifluoromethyl)isoindolin- 1-one |
| 152 | 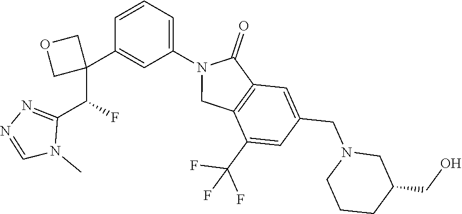 [SMILES: Cn1cnnc1[C@@H](F)C1(c2cccc(N3Cc4c(cc(CN5CCC[C@@H](CO)C5)cc4C(F)(F)F)C3=O)c2)COC1] | 2-(3-(3-((S)-fluoro(4-methyl-4R-1,2,4- triazol-3-yl)methyl)oxetan-3-yl)phenyl)- 6-(((R)-3-(hydroxymethyl)piperidin-1- yl)methyl)-4-(trifluoromethyl)isoindolin- 1-one |
| 153 | 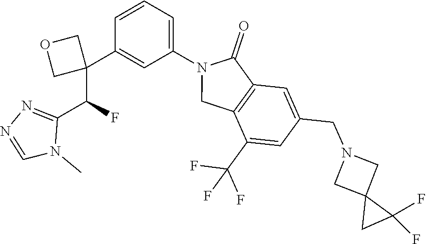 [SMILES: Cn1cnnc1[C@H](F)C1(c2cccc(N3Cc4c(cc(CN5CC6(C5)CC6(F)F)cc4C(F)(F)F)C3=O)c2)COC1] | (R)-6-((1,1-difluoro-5- azaspiro[2.3]hexan-5-yl)methyl)-2-(3- (3-(fluoro(4-methyl-4H-1,2,4-triazol-3- yl)methyl)oxetan-3-yl)phenyl)-4- (trifluoromethyl)isoindolin-1-one |
| 154 | 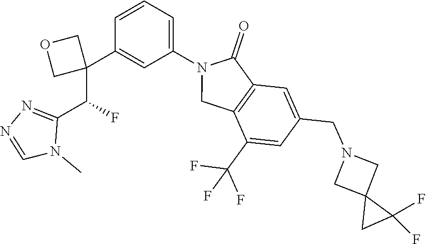 [SMILES: Cn1cnnc1[C@@H](F)C1(c2cccc(N3Cc4c(cc(CN5CC6(C5)CC6(F)F)cc4C(F)(F)F)C3=O)c2)COC1] | (S)-6-((1,1-difluoro-5- azaspiro[2.3]hexan-5-yl)methyl)-2-(3- (3-(fluoro(4-methyl-4H-1,2,4-triazol-3- yl)methyl)oxetan-3-yl)phenyl)-4- (trifluoromethyl)isoindolin-1-one |
| 155 | 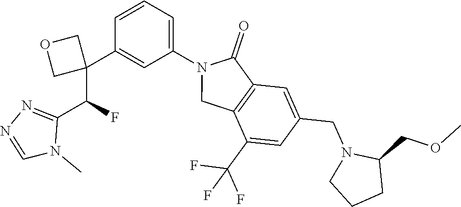 [SMILES: COC[C@H]1CCCN1Cc1cc2c(c(C(F)(F)F)c1)CN(c1cccc(C3([C@@H](F)c4nncn4C)COC3)c1)C2=O] | 2-(3-(3-((R)-fluoro(4-methyl-4H-1,2,4- triazol-3-yl)methyl)oxetan-3-yl)phenyl)- 6-(((R)-2-(methoxymethyl)pyrrolidin-1- yl)methyl)-4-(trifluoromethyl)isoindolin- 1-one |
| 156 | 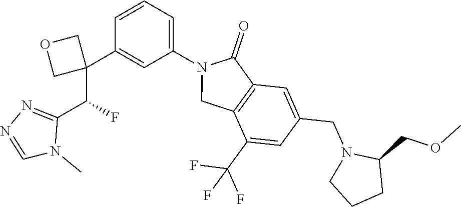 [SMILES: COC[C@H]1CCCN1Cc1cc2c(c(C(F)(F)F)c1)CN(c1cccc(C3([C@H](F)c4nncn4C)COC3)c1)C2=O] | 2-(3-(3-((S)-fluoro(4-methyl-4H-1,2,4- triazol-3-yl)methyl)oxetan-3-yl)phenyl)- 6-(((R)-2-(methoxymethyl)pyrrolidin-1- yl)methyl)-4-(trifluoromethyl)isoindolin- 1-one |
| 157 | 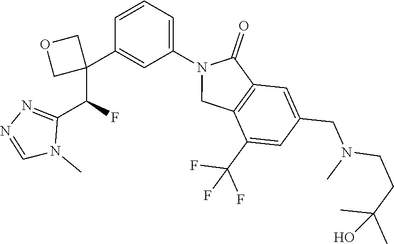 [SMILES: CN(CCC(C)(C)O)Cc1cc2c(c(C(F)(F)F)c1)CN(c1cccc(C3([C@@H](F)c4nncn4C)COC3)c1)C2=O] | (R)-2-(3-(3-(fluoro(4-methyl-4H-1,2,4- triazol-3-yl)methyl)oxetan-3-yl)phenyl)- 6-(((3-hydroxy-3- methylbutyl)(methyl)amino)methyl)-4- (trifluoromethyl)isoindolin-1-one |
| 158 | 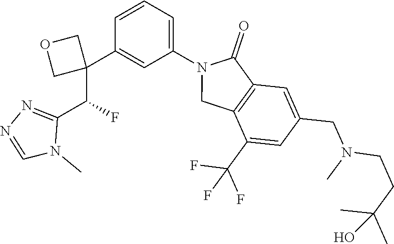 [SMILES: CN(CCC(C)(C)O)Cc1cc2c(c(C(F)(F)F)c1)CN(c1cccc(C3([C@H](F)c4nncn4C)COC3)c1)C2=O] | (S)-2-(3-(3-(fluoro(4-methyl-4H-1,2,4- triazol-3-yl)methyl)oxetan-3-yl)phenyl)- 6-(((3-hydroxy-3- methylbutyl)(methyl)amino)methyl)-4- (trifluoromethyl)isoindolin-1-one |
| 159 | 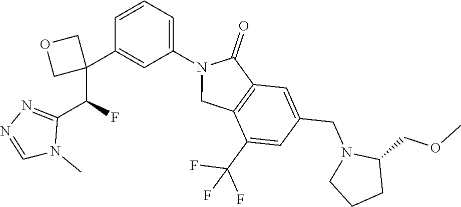 [SMILES: COC[C@@H]1CCCN1Cc1cc2c(c(C(F)(F)F)c1)CN(c1cccc(C3([C@@H](F)c4nncn4C)COC3)c1)C2=O] | 2-(3-(3-((R)-fluoro(4-methyl-4H-1,2,4- triazol-3-yl)methyl)oxetan-3-yl)phenyl)- 6-(((S)-2-(methoxymethyl)pyrrolidin-1- yl)methyl)-4-(trifluoromethyl)isoindolin- 1-one |
| 160 | 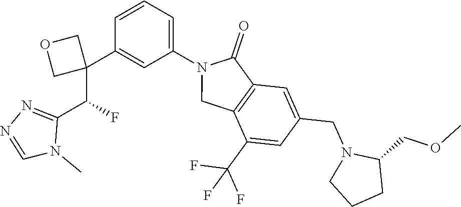 [SMILES: COC[C@@H]1CCCN1Cc1cc2c(c(C(F)(F)F)c1)CN(c1cccc(C3([C@H](F)c4nncn4C)COC3)c1)C2=O] | 2-(3-(3-((S)-fluoro(4-methyl-4H-1,2,4- triazol-3-yl)methyl)oxetan-3-yl)phenyl)- 6-(((S)-2-(methoxymethyl)pyrrolidin-1- yl)methyl)-4-(trifluoromethyl)isoindolin- 1-one |
| 161 | 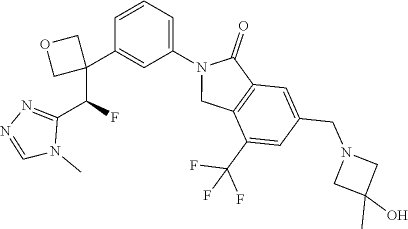 [SMILES: Cn1cnnc1[C@H](F)C1(c2cccc(N3Cc4c(cc(CN5CC(C)(O)C5)cc4C(F)(F)F)C3=O)c2)COC1] | (R)-2-(3-(3-(fluoro(4-methyl-4H-1,2,4- triazol-3-yl)methyl)oxetan-3-yl)phenyl)- 6-((3-hydroxy-3-methylazetidin-1- yl)methyl)-4-(trifluoromethyl)isoindolin- 1-one |
| 162 | 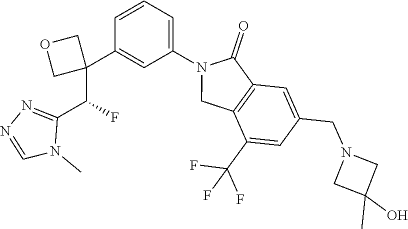 [SMILES: Cn1cnnc1[C@@H](F)C1(c2cccc(N3Cc4c(cc(CN5CC(C)(O)C5)cc4C(F)(F)F)C3=O)c2)COC1] | (S)-2-(3-(3-(fluoro(4-methyl-4H-1,2,4- triazol-3-yl)methyl)oxetan-3-yl)phenyl)- 6-((3-hydroxy-3-methylazetidin-1- yl)methyl)-4-(trifluoromethyl)isoindolin- 1-one |
| 163 | 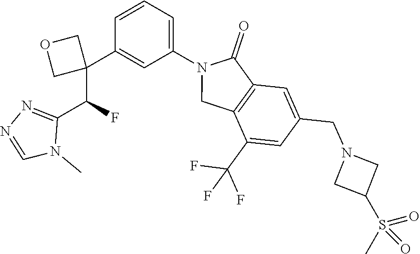 [SMILES: Cn1cnnc1[C@H](F)C1(c2cccc(N3Cc4c(cc(CN5CC(S(C)(=O)=O)C5)cc4C(F)(F)F)C3=O)c2)COC1] | (R)-2-(3-(3-(fluoro(4-methyl-4H-1,2,4- triazol-3-yl)methyl)oxetan-3-yl)phenyl)- 6-((3-(methylsulfonyl)azetidin-1- yl)methyl)-4-(trifluoromethyl)isoindolin- 1-one |
| 164 | 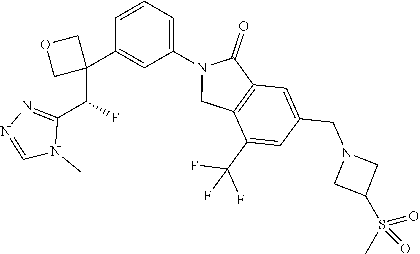 [SMILES: Cn1cnnc1[C@@H](F)C1(c2cccc(N3Cc4c(cc(CN5CC(S(C)(=O)=O)C5)cc4C(F)(F)F)C3=O)c2)COC1] | (S)-2-(3-(3-(fluoro(4-methyl-4H-1,2,4- triazol-3-yl)methyl)oxetan-3-yl)phenyl)- 6-((3-(methylsulfonyl)azetidin-1- yl)methyl)-4-(trifluoromethyl)isoindolin- 1-one |
| 165 | 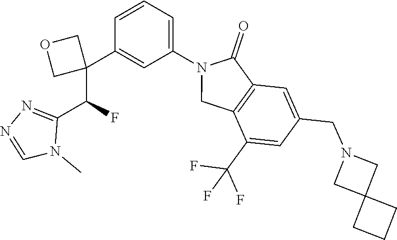 [SMILES: Cn1cnnc1[C@H](F)C1(c2cccc(N3Cc4c(cc(CN5CC6(CCC6)C5)cc4C(F)(F)F)C3=O)c2)COC1] | (R)-6-(2-azaspiro[3.3]heptan-2- ylmethyl)-2-(3-(3-(fluoro(4-methyl-4H- 1,2,4-triazol-3-yl)methyl)oxetan-3- yl)phenyl)-4-(trifluoromethyl)isoindolin- 1-one |
| 166 | 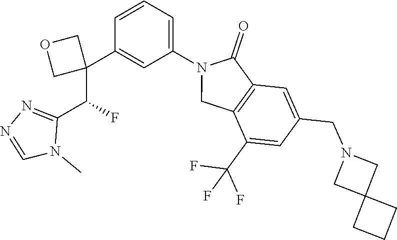 [SMILES: Cn1cnnc1[C@@H](F)C1(c2cccc(N3Cc4c(cc(CN5CC6(CCC6)C5)cc4C(F)(F)F)C3=O)c2)COC1] | (S)-6-(2-azaspiro[3.3]heptan-2- ylmethyl)-2-(3-(3-(fluoro(4-methyl-4H- 1,2,4-triazol-3-yl)methyl)oxetan-3- yl)phenyl)-4-(trifluoromethyl)isoindolin- 1-one |
| 167 | 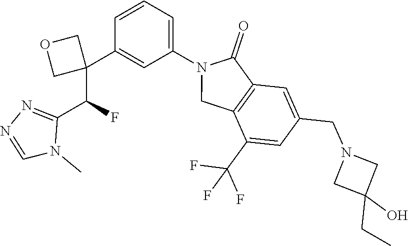 [SMILES: CCC1(O)CN(Cc2cc3c(c(C(F)(F)F)c2)CN(c2cccc(C4([C@@H](F)c5nncn5C)COC4)c2)C3=O)C1] | (R)-6-((3-ethyl-3-hydroxyazetidin-1- yl)methyl)-2-(3-(3-(fluoro(4-methyl-4H- 1,2,4-triazol-3-yl)methyl)oxetan-3- yl)phenyl)-4-(trifluoromethyl)isoindolin- 1-one |
| 168 | 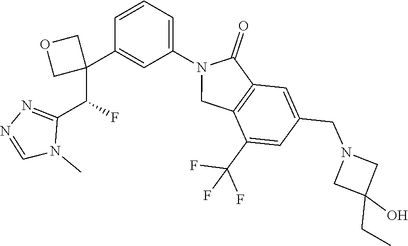 [SMILES: CCC1(O)CN(Cc2cc3c(c(C(F)(F)F)c2)CN(c2cccc(C4([C@H](F)c5nncn5C)COC4)c2)C3=O)C1] | (S)-6-((3-ethyl-3-hydroxyazetidin-1- yl)methyl)-2-(3-(3-(fluoro(4-methyl-4H- 1,2,4-triazol-3-yl)methyl)oxetan-3- yl)phenyl)-4-(trifluoromethyl)isoindolin- 1-one |
| 169 | 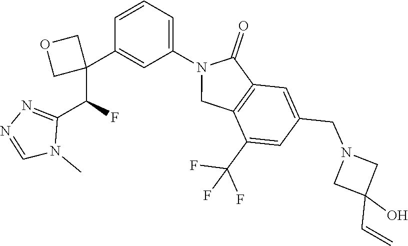 [SMILES: C=CC1(O)CN(Cc2cc3c(c(C(F)(F)F)c2)CN(c2cccc(C4([C@@H](F)c5nncn5C)COC4)c2)C3=O)C1] | (R)-2-(3-(3-(fluoro(4-methyl-4H-1,2,4- triazol-3-yl)methyl)oxetan-3-yl)phenyl)- 6-((3-hydroxy-3-vinylazetidin-1- yl)methyl)-4-(trifluoromethyl)isoindolin- 1-one |
| 170 | 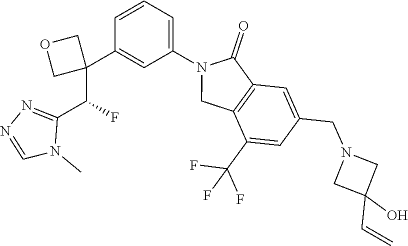 [SMILES: C=CC1(O)CN(Cc2cc3c(c(C(F)(F)F)c2)CN(c2cccc(C4([C@H](F)c5nncn5C)COC4)c2)C3=O)C1] | (S)-2-(3-(3-(fluoro(4-methyl-4H-1,2,4- triazol-3-yl)methyl)oxetan-3-yl)phenyl)- 6-((3-hydroxy-3-vinylazetidin-1- yl)methyl)-4-(trifluoromethyl)isoindolin- 1-one |
| 171 | 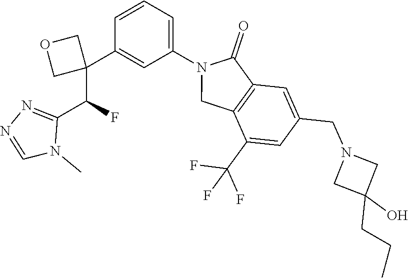 [SMILES: CCCC1(O)CN(Cc2cc3c(c(C(F)(F)F)c2)CN(c2cccc(C4([C@@H](F)c5nncn5C)COC4)c2)C3=O)C1] | (R)-2-(3-(3-(fluoro(4-methyl-4H-1,2,4- triazol-3-yl)methyl)oxetan-3-yl)phenyl)- 6-((3-hydroxy-3-propylazetidin-1- yl)methyl)-4-(trifluoromethyl)isoindolin- 1-one |
| 172 | 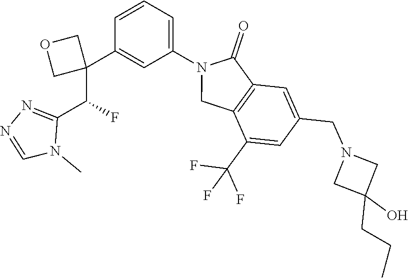 [SMILES: CCCC1(O)CN(Cc2cc3c(c(C(F)(F)F)c2)CN(c2cccc(C4([C@H](F)c5nncn5C)COC4)c2)C3=O)C1] | (S)-2-(3-(3-(fluoro(4-methyl-4H-1,2,4- triazol-3-yl)methyl)oxetan-3-yl)phenyl)- 6-((3-hydroxy-3-propylazetidin-1- yl)methyl)-4-(trifluoromethyl)isoindolin- 1-one |
| 173 | 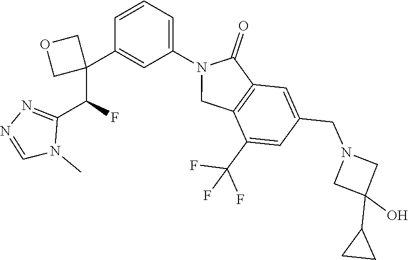 [SMILES: Cn1cnnc1[C@H](F)C1(c2cccc(N3Cc4c(cc(CN5CC(O)(C6CC6)C5)cc4C(F)(F)F)C3=O)c2)COC1] | (R)-6-((3-cyclopropyl-3- hydroxyazetidin-1-yl)methyl)-2-(3-(3- (fluoro(4-methyl-4H-1,2,4-triazol-3- yl)methyl)oxetan-3-yl)phenyl)-4- (trifluoromethyl)isoindolin-1-one |
| 174 | 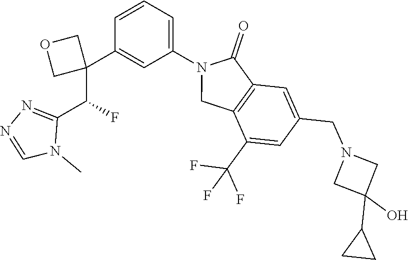 [SMILES: Cn1cnnc1[C@@H](F)C1(c2cccc(N3Cc4c(cc(CN5CC(O)(C6CC6)C5)cc4C(F)(F)F)C3=O)c2)COC1] | (S)-6-((3-cyclopropyl-3- hydroxyazetidin-1-yl)methyl)-2-(3-(3- (fluoro(4-methyl-4H-1,2,4-triazol-3- yl)methyl)oxetan-3-yl)phenyl)-4- (trifluoromethyl)isoindolin-1-one |
| 175 | 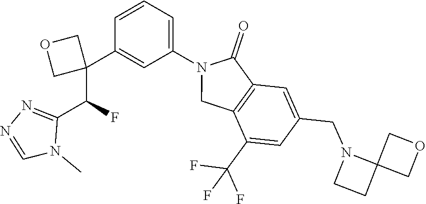 [SMILES: Cn1cnnc1[C@H](F)C1(c2cccc(N3Cc4c(cc(CN5CCC56COC6)cc4C(F)(F)F)C3=O)c2)COC1] | (R)-6-(6-oxa-1-azaspiro[3.3]heptan-1- ylmethyl)-2-(3-(3-(fluoro(4-methyl-4H- 1,2,4-triazol-3-yl)methyl)oxetan-3- yl)phenyl)-4-(trifluoromethyl)isoindolin- 1-one |
| 176 | 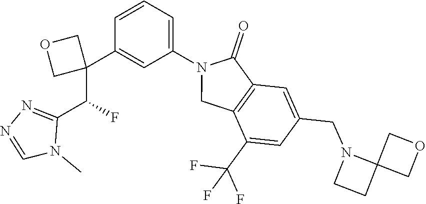 [SMILES: Cn1cnnc1[C@@H](F)C1(c2cccc(N3Cc4c(cc(CN5CCC56COC6)cc4C(F)(F)F)C3=O)c2)COC1] | (S)-6-(6-oxa-1-azaspiro[3.3]heptan-1- ylmethyl)-2-(3-(3-(fluoro(4-methyl-4H- 1,2,4-triazol-3-yl)methyl)oxetan-3- yl)phenyl)-4-(trifluoromethyl)isoindolin- 1-one |
| 177 | 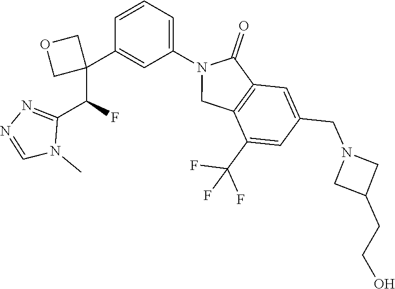 [SMILES: Cn1cnnc1[C@H](F)C1(c2cccc(N3Cc4c(cc(CN5CC(CCO)C5)cc4C(F)(F)F)C3=O)c2)COC1] | (R)-2-(3-(3-(fluoro(4-methyl-4H-1,2,4- triazol-3-yl)methyl)oxetan-3-yl)phenyl)- 6-((3-(2-hydroxyethyl)azetidin-1- yl)methyl)-4-(trifluoromethyl)isoindolin- 1-one |
| 178 | 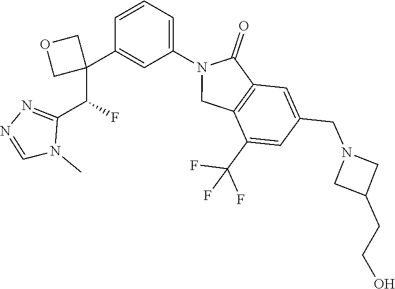 [SMILES: Cn1cnnc1[C@@H](F)C1(c2cccc(N3Cc4c(cc(CN5CC(CCO)C5)cc4C(F)(F)F)C3=O)c2)COC1] | (S)-2-(3-(3-(fluoro(4-methyl-4H-1,2,4- triazol-3-yl)methyl)oxetan-3-yl)phenyl)- 6-((3-(2-hydroxyethyl)azetidin-1- yl)methyl)-4-(trifluoromethyl)isoindolin- 1-one |
| 179 | 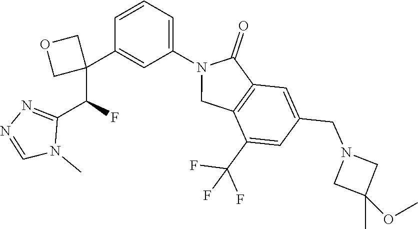 [SMILES: COC1(C)CN(Cc2cc3c(c(C(F)(F)F)c2)CN(c2cccc(C4([C@@H](F)c5nncn5C)COC4)c2)C3=O)C1] | (R)-2-(3-(3-(fluoro(4-methyl-4H-1,2,4- triazol-3-yl)methyl)oxetan-3-yl)phenyl)- 6-((3-methoxy-3-methylazetidin-1- yl)methyl)-4-(trifluoromethyl)isoindolin- 1-one |
| 180 | 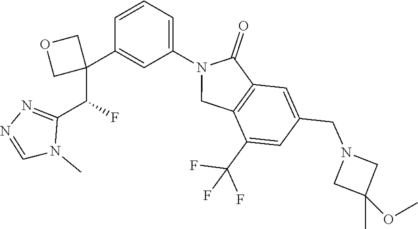 [SMILES: COC1(C)CN(Cc2cc3c(c(C(F)(F)F)c2)CN(c2cccc(C4([C@H](F)c5nncn5C)COC4)c2)C3=O)C1] | (S)-2-(3-(3-(fluoro(4-methyl-4H-1,2,4- triazol-3-yl)methyl)oxetan-3-yl)phenyl)- 6-((3-methoxy-3-methylazetidin-1- yl)methyl)-4-(trifluoromethyl)isoindolin- 1-one |
| 181 | 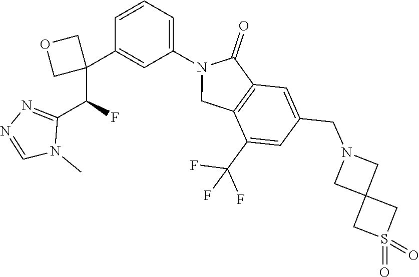 [SMILES: Cn1cnnc1[C@H](F)C1(c2cccc(N3Cc4c(cc(CN5CC6(C5)CS(=O)(=O)C6)cc4C(F)(F)F)C3=O)c2)COC1] | (R)-6-((2,2-dioxido-2-thia-6- azaspiro[3.3]heptan-6-yl)methyl)-2-(3- (3-(fluoro(4-methyl-4H-1,2,4-triazol-3- yl)methyl)oxetan-3-yl)phenyl)-4- (trifluoromethyl)isoindolin-1-one |
| 182 | 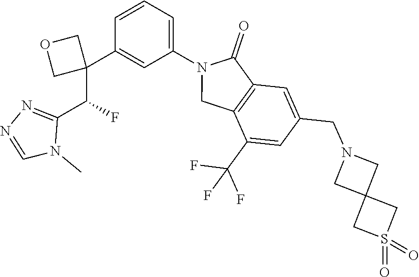 [SMILES: Cn1cnnc1[C@@H](F)C1(c2cccc(N3Cc4c(cc(CN5CC6(C5)CS(=O)(=O)C6)cc4C(F)(F)F)C3=O)c2)COC1] | (S)-6-((2,2-dioxido-2-thia-6- azaspiro[3.3]heptan-6-yl)methyl)-2-(3- (3-(fluoro(4-methyl-4H-1,2,4-triazol-3- yl)methyl)oxetan-3-yl)phenyl)-4- (trifluoromethyl)isoindolin-1-one |
| 183 | 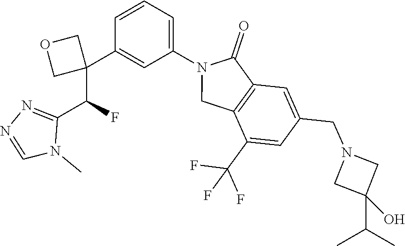 [SMILES: CC(C)C1(O)CN(Cc2cc3c(c(C(F)(F)F)c2)CN(c2cccc(C4([C@@H](F)c5nncn5C)COC4)c2)C3=O)C1] | (R)-2-(3-(3-(fluoro(4-methyl-4H-1,2,4- triazol-3-yl)methyl)oxetan-3-yl)phenyl)- 6-((3-hydroxy-3-isopropylazetidin-1- yl)methyl)-4-(trifluoromethyl)isoindolin- 1-one |
| 184 | 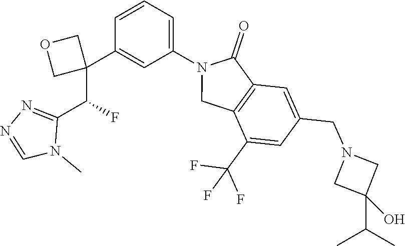 [SMILES: CC(C)C1(O)CN(Cc2cc3c(c(C(F)(F)F)c2)CN(c2cccc(C4([C@H](F)c5nncn5C)COC4)c2)C3=O)C1] | (S)-2-(3-(3-(fluoro(4-methyl-4H-1,2,4- triazol-3-yl)methyl)oxetan-3-yl)phenyl)- 6-((3-hydroxy-3-isopropylazetidin-1- yl)methyl)-4-(trifluoromethyl)isoindolin- 1-one |
| 185 | 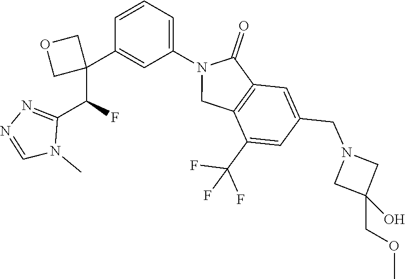 [SMILES: COCC1(O)CN(Cc2cc3c(c(C(F)(F)F)c2)CN(c2cccc(C4([C@@H](F)c5nncn5C)COC4)c2)C3=O)C1] | (R)-2-(3-(3-(fluoro(4-methyl-4H-1,2,4- triazol-3-yl)methyl)oxetan-3-yl)phenyl)- 6-((3-hydroxy-3- (methoxymethyl)azetidin-1-yl)methyl)- 4-(trifluoromethyl)isoindolin-1-one |
| 186 | 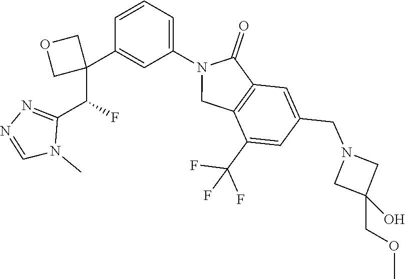 [SMILES: COCC1(O)CN(Cc2cc3c(c(C(F)(F)F)c2)CN(c2cccc(C4([C@H](F)c5nncn5C)COC4)c2)C3=O)C1] | (S)-2-(3-(3-(fluoro(4-methyl-4H-1,2,4- triazol-3-yl)methyl)oxetan-3-yl)phenyl)- 6-((3-hydroxy-3- (methoxymethyl)azetidin-1-yl)methyl)- 4-(trifluoromethyl)isoindolin-1-one |
| 187 | 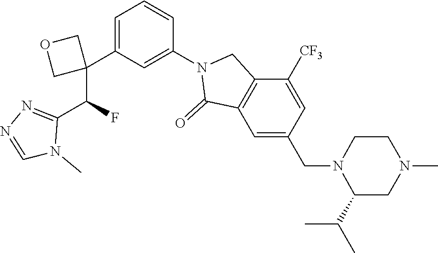 [SMILES: CC(C)[C@H]1CN(C)CCN1Cc1cc2c(c(C(F)(F)F)c1)CN(c1cccc(C3([C@@H](F)c4nncn4C)COC3)c1)C2=O] | 2-(3-(3-((R)-fluoro(4-methyl-4H-1,2,4- triazol-3-yl)methyl)oxetan-3-yl)phenyl)- 6-(((S)-2-isopropyl-4-methylpiperazin- 1-yl)methyl)-4- (trifluoromethyl)isoindolin-1-one |
| 188 | 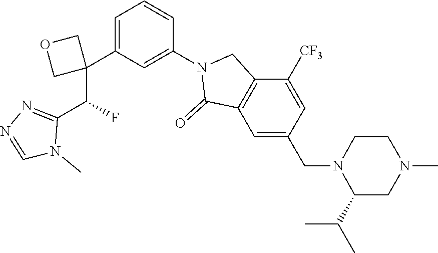 [SMILES: CC(C)[C@H]1CN(C)CCN1Cc1cc2c(c(C(F)(F)F)c1)CN(c1cccc(C3([C@H](F)c4nncn4C)COC3)c1)C2=O] | 2-(3-(3-((S)-fluoro(4-methyl-4H-1,2,4- triazol-3-yl)methyl)oxetan-3-yl)phenyl)- 6-(((S)-2-isopropyl-4-methylpiperazin- 1-yl)methyl)-4- (trifluoromethyl)isoindolin-1-one |
| 189 | 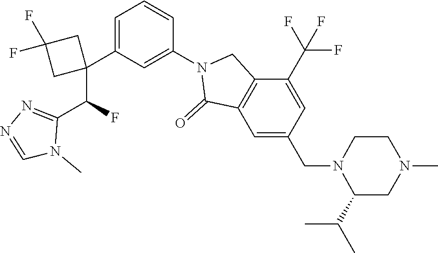 [SMILES: CC(C)[C@H]1CN(C)CCN1Cc1cc2c(c(C(F)(F)F)c1)CN(c1cccc(C3([C@@H](F)c4nncn4C)CC(F)(F)C3)c1)C2=O] | 2-(3-(3,3-difluoro-1-((R)-fluoro(4- methyl-4H-1,2,4-triazol-3- yl)methyl)cyclobutyl)phenyl)-6-(((S)-2- isopropyl-4-methylpiperazin-1- yl)methyl)-4-(trifluoromethyl)isoindolin- 1-one |
| 190 | 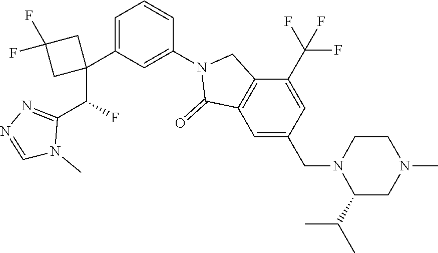 [SMILES: CC(C)[C@H]1CN(C)CCN1Cc1cc2c(c(C(F)(F)F)c1)CN(c1cccc(C3([C@H](F)c4nncn4C)CC(F)(F)C3)c1)C2=O] | 2-(3-(3,3-difluoro-1-((S)-fluoro(4- methyl-4H-1,2,4-triazol-3- yl)methyl)cyclobutyl)phenyl)-6-(((S)-2- isopropyl-4-methylpiperazin-1- yl)methyl)-4-(trifluoromethyl)isoindolin- 1-one |
| 191 | 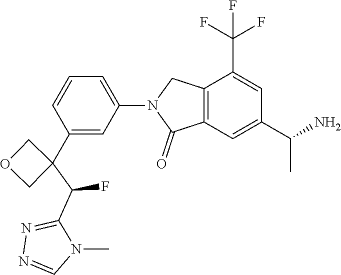 [SMILES: C[C@@H](N)c1cc2c(c(C(F)(F)F)c1)CN(c1cccc(C3([C@@H](F)c4nncn4C)COC3)c1)C2=O] | 6-((R)-1-aminoethyl)-2-(3-(3-((R)- fluoro(4-methyl-4H-1,2,4-triazol-3- yl)methyl)oxetan-3-yl)phenyl)-4- (trifluoromethyl)isoindolin-1-one |
| 192 | 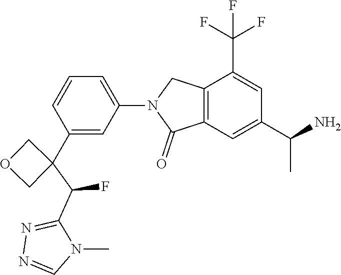 [SMILES: C[C@H](N)c1cc2c(c(C(F)(F)F)c1)CN(c1cccc(C3([C@@H](F)c4nncn4C)COC3)c1)C2=O] | 6-((S)-1-aminoethyl)-2-(3-(3-((R)- fluoro(4-methyl-4H-1,2,4-triazol-3- yl)methyl)oxetan-3-yl)phenyl)-4- (trifluoromethyl)isoindolin-1-one |
| 193 | 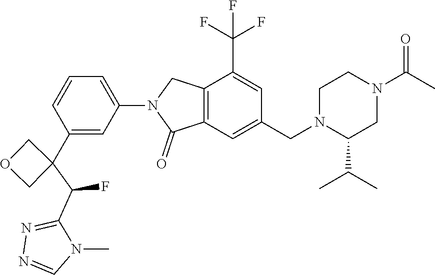 [SMILES: CC(=O)N1CCN(Cc2cc3c(c(C(F)(F)F)c2)CN(c2cccc(C4([C@@H](F)c5nncn5C)COC4)c2)C3=O)[C@@H](C(C)C)C1] | 6-(((S)-4-acetyl-2-isopropylpiperazin-1- yl)methyl)-2-(3-(3-((R)-fluoro(4-methyl- 4H-1,2,4-triazol-3-yl)methyl)oxetan-3- yl)phenyl)-4-(trifluoromethyl)isoindolin- 1-one |
| 194 | 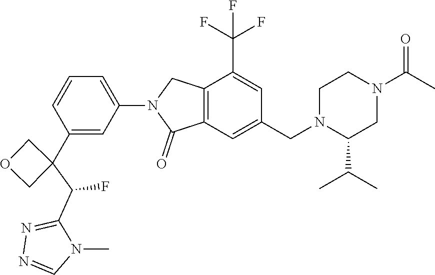 [SMILES: CC(=O)N1CCN(Cc2cc3c(c(C(F)(F)F)c2)CN(c2cccc(C4([C@H](F)c5nncn5C)COC4)c2)C3=O)[C@@H](C(C)C)C1] | 6-(((S)-4-acetyl-2-isopropylpiperazin-1- yl)methyl)-2-(3-(3-((S)-fluoro(4-methyl- 4H-1,2,4-triazol-3-yl)methyl)oxetan-3- yl)phenyl)-4-(trifluoromethyl)isoindolin- 1-one |
| 195 | 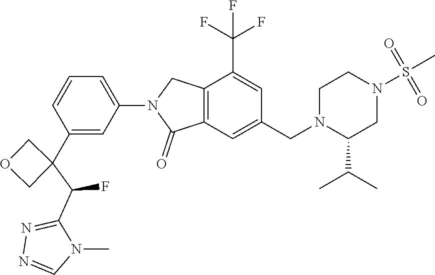 [SMILES: CC(C)[C@H]1CN(S(C)(=O)=O)CCN1Cc1cc2c(c(C(F)(F)F)c1)CN(c1cccc(C3([C@@H](F)c4nncn4C)COC3)c1)C2=O] | 2-(3-(3-((R)-fluoro(4-methyl-4H-1,2,4- triazol-3-yl)methyl)oxetan-3-yl)phenyl)- 6-(((S)-2-isopropyl-4- (methylsulfonyl)piperazin-1-yl)methyl)- 4-(trifluoromethyl)isoindolin-1-one |
| 196 | 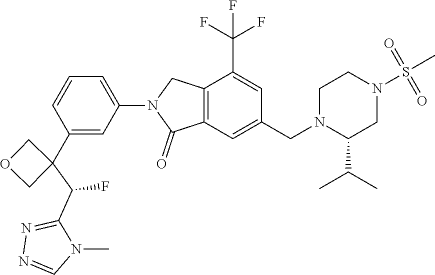 [SMILES: CC(C)[C@H]1CN(S(C)(=O)=O)CCN1Cc1cc2c(c(C(F)(F)F)c1)CN(c1cccc(C3([C@H](F)c4nncn4C)COC3)c1)C2=O] | 2-(3-(3-((S)-fluoro(4-methyl-4H-1,2,4- triazol-3-yl)methyl)oxetan-3-yl)phenyl)- 6-(((S)-2-isopropyl-4- (methylsulfonyl)piperazin-1-yl)methyl)- 4-(trifluoromethyl)isoindolin-1-one |
| 197 | 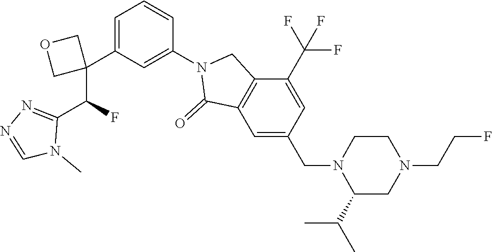 [SMILES: CC(C)[C@H]1CN(CCF)CCN1Cc1cc2c(c(C(F)(F)F)c1)CN(c1cccc(C3([C@@H](F)c4nncn4C)COC3)c1)C2=O] | 2-(3-(3-((R)-fluoro(4-methyl-4H-1,2,4- triazol-3-yl)methyl)oxetan-3-yl)phenyl)- 6-(((S)-4-(2-fluoroethyl)-2- isopropylpiperazin-1-yl)methyl)-4- (trifluoromethyl)isoindolin-1-one |
| 198 | 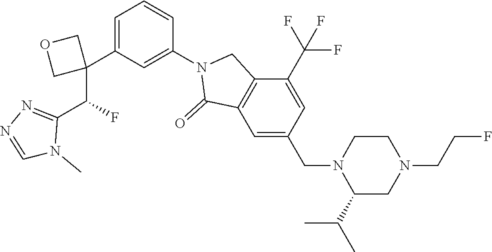 [SMILES: CC(C)[C@H]1CN(CCF)CCN1Cc1cc2c(c(C(F)(F)F)c1)CN(c1cccc(C3([C@H](F)c4nncn4C)COC3)c1)C2=O] | 2-(3-(3-((S)-fluoro(4-methyl-4H-1,2,4- triazol-3-yl)methyl)oxetan-3-yl)phenyl)- 6-(((S)-4-(2-fluoroethyl)-2- isopropylpiperazin-1-yl)methyl)-4- (trifluoromethyl)isoindolin-1-one |
| 199 | 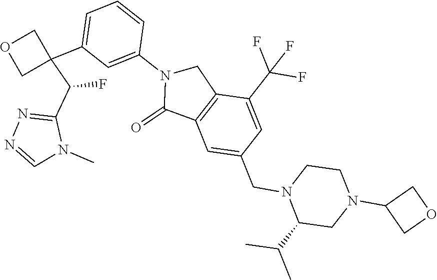 [SMILES: CC(C)[C@H]1CN(C2COC2)CCN1Cc1cc2c(c(C(F)(F)F)c1)CN(c1cccc(C3([C@H](F)c4nncn4C)COC3)c1)C2=O] | 2-(3-(3-((S)-fluoro(4-methyl-4H-1,2,4- triazol-3-yl)methyl)oxetan-3-yl)phenyl)- 6-(((S)-2-isopropyl-4-(oxetan-3- yl)piperazin-1-yl)methyl)-4- (trifluoromethyl)isoindolin-1-one |
| 200 | 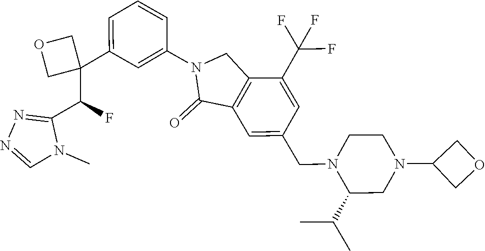 [SMILES: CC(C)[C@H]1CN(C2COC2)CCN1Cc1cc2c(c(C(F)(F)F)c1)CN(c1cccc(C3([C@@H](F)c4nncn4C)COC3)c1)C2=O] | 2-(3-(3-((R)-fluoro(4-methyl-4H-1,2,4- triazol-3-yl)methyl)oxetan-3-yl)phenyl)- 6-(((S)-2-isopropyl-4-(oxetan-3- yl)piperazin-1-yl)methyl)-4- (trifluoromethyl)isoindolin-1-one |
| 201 | 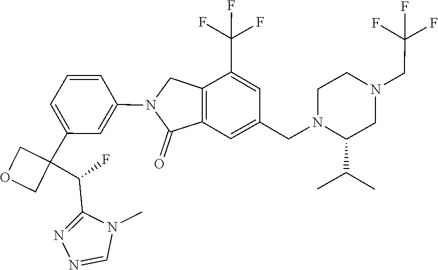 [SMILES: CC(C)[C@H]1CN(CC(F)(F)F)CCN1Cc1cc2c(c(C(F)(F)F)c1)CN(c1cccc(C3([C@H](F)c4nncn4C)COC3)c1)C2=O] | 2-(3-(3-((S)-fluoro(4-methyl-4H-1,2,4- triazol-3-yl)methyl)oxetan-3-yl)phenyl)- 6-(((S)-2-isopropyl-4-(2,2,2- trifluoroethyl)piperazin-1-yl)methyl)-4- (trifluoromethyl)isoindolin-1-one |
| 202 | 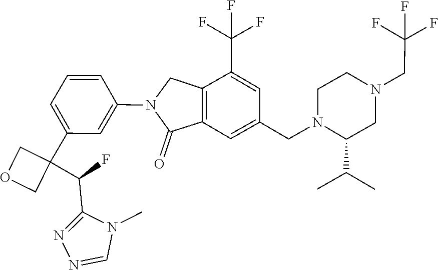 [SMILES: CC(C)[C@H]1CN(CC(F)(F)F)CCN1Cc1cc2c(c(C(F)(F)F)c1)CN(c1cccc(C3([C@@H](F)c4nncn4C)COC3)c1)C2=O] | 2-(3-(3-((R)-fluoro(4-methyl-4H-1,2,4- triazol-3-yl)methyl)oxetan-3-yl)phenyl)- 6-(((S)-2-isopropyl-4-(2,2,2- trifluoroethyl)piperazin-1-yl)methyl)-4- (trifluoromethyl)isoindolin-1-one |
| 203 | 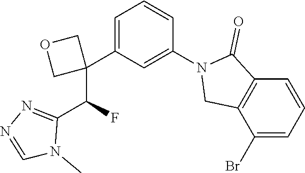 [SMILES: Cn1cnnc1[C@H](F)C1(c2cccc(N3Cc4c(Br)cccc4C3=O)c2)COC1] | (R)-4-bromo-2-(3-(3-(fluoro(4-methyl- 4H-1,2,4-triazol-3-yl)methyl)oxetan-3- yl)phenyl)isoindolin-1-one |
| 204 | 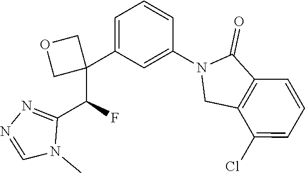 [SMILES: Cn1cnnc1[C@H](F)C1(c2cccc(N3Cc4c(Cl)cccc4C3=O)c2)COC1] | (R)-4-chloro-2-(3-(3-(fluoro(4-methyl- 4H-1,2,4-triazol-3-yl)methyl)oxetan-3- yl)phenyl)isoindolin-1-one |
| 205 | 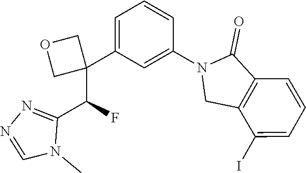 [SMILES: Cn1cnnc1[C@H](F)C1(c2cccc(N3Cc4c(I)cccc4C3=O)c2)COC1] | (R)-2-(3-(3-(fluoro(4-methyl-4H-1,2,4- triazol-3-yl)methyl)oxetan-3-yl)phenyl)- 4-iodoisoindolin-1-one |
| 206 | 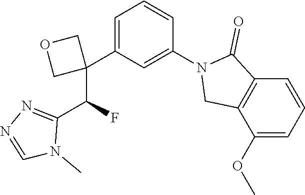 [SMILES: COc1cccc2c1CN(c1cccc(C3([C@@H](F)c4nncn4C)COC3)c1)C2=O] | (R)-2-(3-(3-(fluoro(4-methyl-4H-1,2,4- triazol-3-yl)methyl)oxetan-3-yl)phenyl)- 4-methoxyisoindolin-1-one |
| 207 | 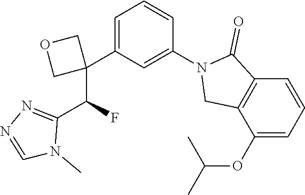 [SMILES: CC(C)Oc1cccc2c1CN(c1cccc(C3([C@@H](F)c4nncn4C)COC3)c1)C2=O] | (R)-2-(3-(3-(fluoro(4-methyl-4H-1,2,4- triazol-3-yl)methyl)oxetan-3-yl)phenyl)- 4-isopropoxyisoindolin-1-one |
| 208 | 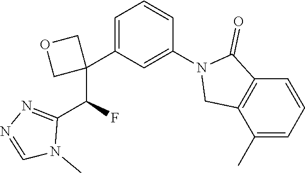 [SMILES: Cc1cccc2c1CN(c1cccc(C3([C@@H](F)c4nncn4C)COC3)c1)C2=O] | (R)-2-(3-(3-(fluoro(4-methyl-4H-1,2,4- triazol-3-yl)methyl)oxetan-3-yl)phenyl)- 4-methylisoindolin-1-one |
| 209 | 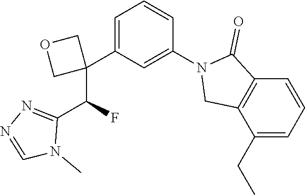 [SMILES: CCc1cccc2c1CN(c1cccc(C3([C@@H](F)c4nncn4C)COC3)c1)C2=O] | R)-2-(3-(3-(fluoro(4-methyl-4H-1,2,4- triazol-3-yl)methyl)oxetan-3-yl)phenyl)- 4-ethylisoindolin-1-one |
| 210 | 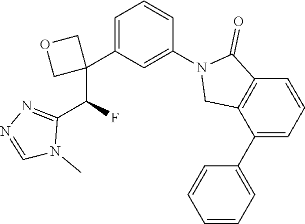 [SMILES: Cn1cnnc1[C@H](F)C1(c2cccc(N3Cc4c(cccc4-c4ccccc4)C3=O)c2)COC1] | (R)-2-(3-(3-(fluoro(4-methyl-4H-1,2,4- triazol-3-yl)methyl)oxetan-3-yl)phenyl)- 4-phenylisoindolin-1-one |
| 211 | 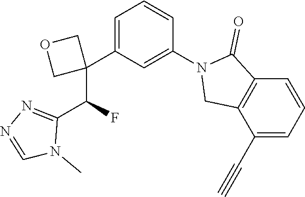 [SMILES: C#Cc1cccc2c1CN(c1cccc(C3([C@@H](F)c4nncn4C)COC3)c1)C2=O] | (R)-4-ethynyl-2-(3-(3-(fluoro(4-methyl- 4H-1,2,4-triazol-3-yl)methyl)oxetan-3- yl)phenyl)isoindolin-1-one |
| 212 | 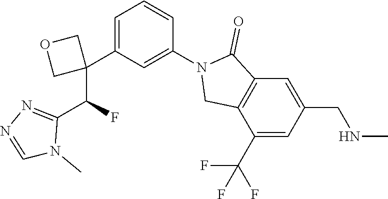 [SMILES: CNCc1cc2c(c(C(F)(F)F)c1)CN(c1cccc(C3([C@@H](F)c4nncn4C)COC3)c1)C2=O] | (R)-2-(3-(3-(fluoro(4-methyl-4H-1,2,4- triazol-3-yl)methyl)oxetan-3-yl)phenyl)- 6-((methylamino)methyl)-4- (trifluoromethyl)isoindolin-1-one |
| 213 | 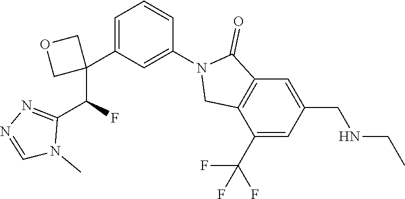 [SMILES: CCNCc1cc2c(c(C(F)(F)F)c1)CN(c1cccc(C3([C@@H](F)c4nncn4C)COC3)c1)C2=O] | (R)-6-((ethylamino)methyl)-2-(3-(3- (fluoro(4-methyl-4H-1,2,4-triazol-3- yl)methyl)oxetan-3-yl)phenyl)-4- (trifluoromethyl)isoindolin-1-one |
| 214 | 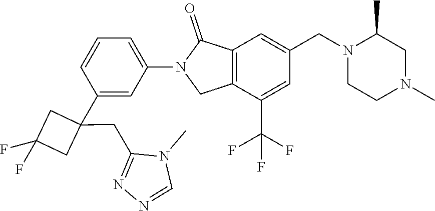 [SMILES: C[C@H]1CN(C)CCN1Cc1cc2c(c(C(F)(F)F)c1)CN(c1cccc(C3(Cc4nncn4C)CC(F)(F)C3)c1)C2=O] | (S)-2-(3-(3,3-difluoro-1-((4-methyl-4H- 1,2,4-triazol-3- yl)methyl)cyclobutyl)phenyl)-6-((2,4- dimethylpiperazin-1-yl)methyl)-4- (trifluoromethyl)isoindolin-1-one |
| 215 | 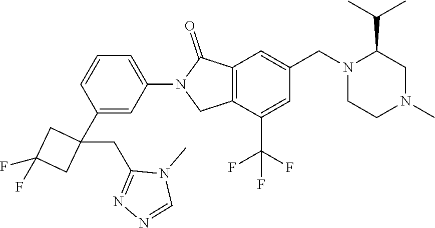 [SMILES: CC(C)[C@H]1CN(C)CCN1Cc1cc2c(c(C(F)(F)F)c1)CN(c1cccc(C3(Cc4nncn4C)CC(F)(F)C3)c1)C2=O] | (S)-2-(3-(3,3-difluoro-1-((4-methyl-4H- 1,2,4-triazol-3- yl)methyl)cyclobutyl)phenyl)-6-((2- isopropyl-4-methylpiperazin-1- yl)methyl)-4-(trifluoromethyl)isoindolin- 1-one |
| 216 | 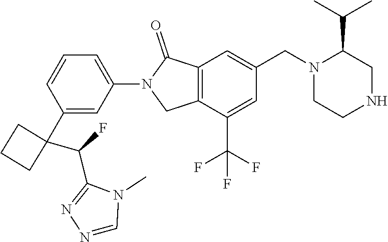 | 2-(3-(1-((R)-fluoro(4-methyl-4H-1,2,4- triazol-3-yl)methyl)cyclobutyl)phenyl)-6- (((S)-2-isopropylpiperazin-1-yl)methyl)- 4-(trifluoromethyl)isoindolin-1-one |
| 217 | 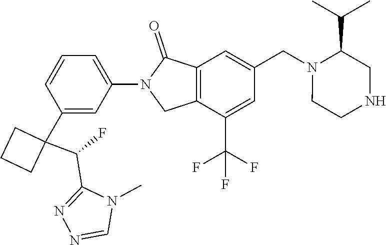 [SMILES: CC(C)[C@H]1CNCCN1Cc1cc2c(c(C(F)(F)F)c1)CN(c1cccc(C3([C@H](F)c4nncn4C)CCC3)c1)C2=O] | 2-(3-(1-((S)-fluoro(4-methyl-4H-1,2,4- triazol-3-yl)methyl)cyclobutyl)phenyl)-6- (((S)-2-isopropylpiperazin-1-yl)methyl)- 4-(trifluoromethyl)isoindolin-1-one |
| 218 | 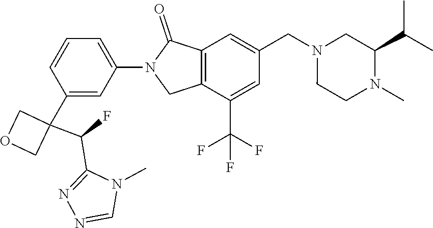 [SMILES: CC(C)[C@@H]1CN(Cc2cc3c(c(C(F)(F)F)c2)CN(c2cccc(C4([C@@H](F)c5nncn5C)COC4)c2)C3=O)CCN1C] | 2-(3-(3-((R)-fluoro(4-methyl-4H-1,2,4- triazol-3-yl)methyl)oxetan-3-yl)phenyl)- 6-(((R)-3-isopropyl-4-methylpiperazin- 1-yl)methyl)-4- (trifluoromethyl)isoindolin-1-one |
| 219 | 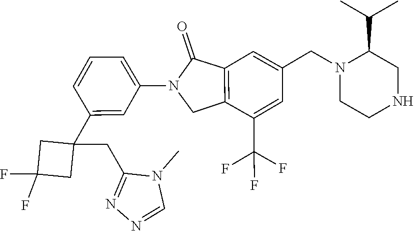 [SMILES: CC(C)[C@H]1CNCCN1Cc1cc2c(c(C(F)(F)F)c1)CN(c1cccc(C3(Cc4nncn4C)CC(F)(F)C3)c1)C2=O] | (S)-2-(3-(3,3-difluoro-1-((4-methyl-4H- 1,2,4-triazol-3- yl)methyl)cyclobutyl)phenyl)-6-((2- isopropylpiperazin-1-yl)methyl)-4- (trifluoromethyl)isoindolin-1-one |
| 220 | 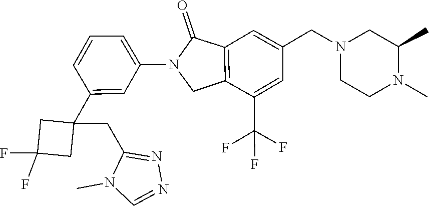 [SMILES: C[C@@H]1CN(Cc2cc3c(c(C(F)(F)F)c2)CN(c2cccc(C4(Cc5nncn5C)CC(F)(F)C4)c2)C3=O)CCN1C] | (R)-2-(3-(3,3-difluoro-1-((4-methyl-4H- 1,2,4-triazol-3- yl)methyl)cyclobutyl)phenyl)-6-((3,4- dimethylpiperazin-1-yl)methyl)-4- (trifluoromethyl)isoindolin-1-one |
| 221 | 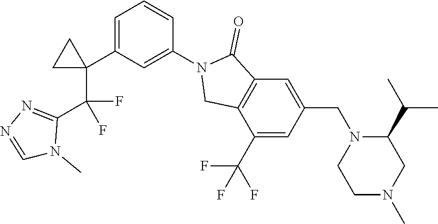 [SMILES: CC(C)[C@H]1CN(C)CCN1Cc1cc2c(c(C(F)(F)F)c1)CN(c1cccc(C3(C(F)(F)c4nncn4C)CC3)c1)C2=O] | (S)-2-(3-(1-(Difluoro(4-methyl-4H-1,2,4- triazol-3-yl)methyl)cyclopropyl)phenyl)- 6-((2-isopropyl-4-methylpiperazin-1- yl)methyl)-4-(trifluoromethyl)isoindolin- 1-one |
| 222 | 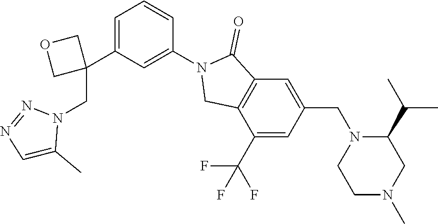 [SMILES: Cc1cnnn1CC1(c2cccc(N3Cc4c(cc(CN5CCN(C)C[C@@H]5C(C)C)cc4C(F)(F)F)C3=O)c2)COC1] | (S)-6-((2-Isopropyl-4-methylpiperazin- 1-yl)methyl)-2-(3-(3-((5-methyl-1H- 1,2,3-triazol-1-yl)methyl)oxetan-3- yl)phenyl)-4-(trifluoromethyl)isoindolin- 1-one |
| 226 | 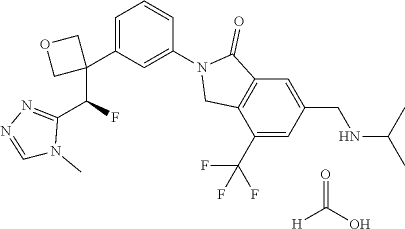 [SMILES: CC(C)NCc1cc2c(c(C(F)(F)F)c1)CN(c1cccc(C3([C@@H](F)c4nncn4C)COC3)c1)C2=O ; O=CO] | (R)-2-(3-(3-(fluoro(4-methyl-4H-1,2,4- triazol-3-yl)methyl)oxetan-3-yl)phenyl)- 6-((isopropylamino)methyl)-4- (trifluoromethyl)isoindolin-1-one formate |
| 227 | 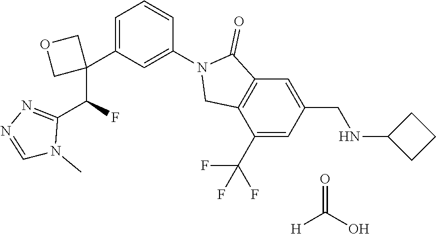 [SMILES: Cn1cnnc1[C@H](F)C1(c2cccc(N3Cc4c(cc(CNC5CCC5)cc4C(F)(F)F)C3=O)c2)COC1 ; O=CO] | (R)-6-((Cyclobutylamino)methyl)-2-(3- (3-(fluoro(4-methyl-4H-1,2,4-triazol-3- yl)methyl)oxetan-3-yl)phenyl)-4- (trifluoromethyl)isoindolin-1-one formate |
| 228 | 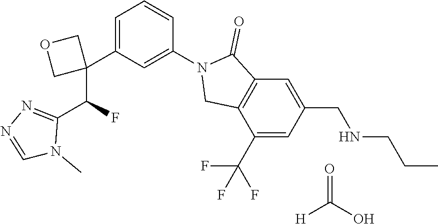 | (R)-2-(3-(3-(fluoro(4-methyl-4H-1,2,4- triazol-3-yl)methyl)oxetan-3-yl)phenyl)- 6-((propylamino)methyl)-4- (trifluoromethyl)isoindolin-1-one formate |
| 229 | 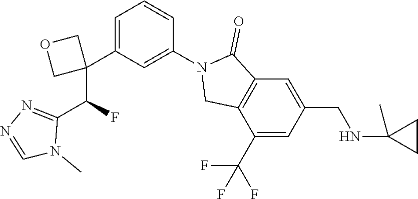 [SMILES: Cn1cnnc1[C@H](F)C1(c2cccc(N3Cc4c(cc(CNC5(C)CC5)cc4C(F)(F)F)C3=O)c2)COC1] | (R)-2-(3-(3-(fluoro(4-methyl-4H-1,2,4- triazol-3-yl)methyl)oxetan-3-yl)phenyl)- 6-(((1- methylcyclopropyl)amino)methyl)-4- (trifluoromethyl)isoindolin-1-one |
| 230 | 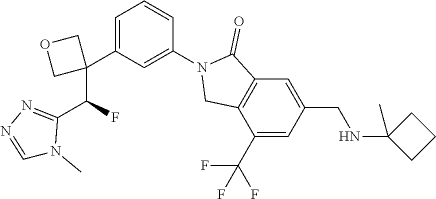 [SMILES: Cn1cnnc1[C@H](F)C1(c2cccc(N3Cc4c(cc(CNC5(C)CCC5)cc4C(F)(F)F)C3=O)c2)COC1] | (R)-2-(3-(3-(fluoro(4-methyl-4H-1,2,4- triazol-3-yl)methyl)oxetan-3-yl)phenyl)- 6-(((1-methylcyclobutyl)amino)methyl)- 4-(trifluoromethyl)isoindolin-1-one |
| 231 | 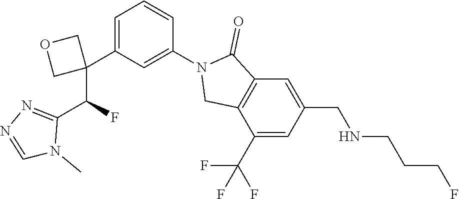 [SMILES: Cn1cnnc1[C@H](F)C1(c2cccc(N3Cc4c(cc(CNCCCF)cc4C(F)(F)F)C3=O)c2)COC1] | (R)-2-(3-(3-(fluoro(4-methyl-4H-1,2,4- triazol-3-yl)methyl)oxetan-3-yl)phenyl)- 6-(((3-fluoropropyl)amino)methyl)-4- (trifluoromethyl)isoindolin-1-one |
| 275 | 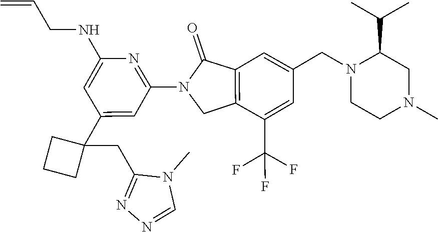 [SMILES: C=CCNc1cc(C2(Cc3nncn3C)CCC2)cc(N2Cc3c(cc(CN4CCN(C)C[C@@H]4C(C)C)cc3C(F)(F)F)C2=O)n1] | (S)-2-(6-(allylamino)-4-(1-((4-methyl- 4H-1,2,4-triazol-3- yl)methyl)cyclobutyl)pyridin-2-yl)-6-((2- isopropyl-4-methylpiperazin-1- yl)methyl)-4-(trifluoromethyl)isoindolin- 1-one |
| 276 | 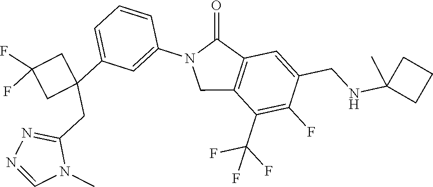 [SMILES: Cn1cnnc1CC1(c2cccc(N3Cc4c(cc(CNC5(C)CCC5)c(F)c4C(F)(F)F)C3=O)c2)CC(F)(F)C1] | 2-(3-(3,3-difluoro-1-((4-methyl-4H- 1,2,4-triazol-3- yl)methyl)cyclobutyl)phenyl)-5-fluoro-6- (((1-methylcyclobutyl)amino)methyl)-4- (trifluoromethyl)isoindolin-1-one |
| 277 | 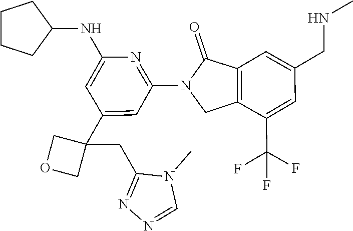 [SMILES: CNCc1cc2c(c(C(F)(F)F)c1)CN(c1cc(C3(Cc4nncn4C)COC3)cc(NC3CCCC3)n1)C2=O] | 2-(6-(cyclopentylamino)-4-(3-((4- methyl-4H-1,2,4-triazol-3- yl)methyl)oxetan-3-yl)pyridin-2-yl)-6- ((methylamino)methyl)-4- (trifluoromethyl)isoindolin-1-one |
| 278 | 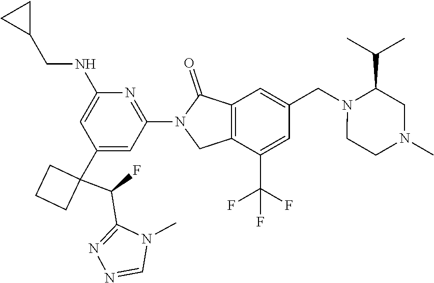 [SMILES: CC(C)[C@H]1CN(C)CCN1Cc1cc2c(c(C(F)(F)F)c1)CN(c1cc(C3([C@@H](F)c4nncn4C)CCC3)cc(NCC3CC3)n1)C2=O] | 2-(6-((cyclopropylmethyl)amino)-4-(1- ((R)-fluoro(4-methy-4H-1,2,4-triazol-3- yl)methyl)cyclobutyl)pyridin-2-yl)-6- (((S)-2-isopropyl-4-methylpiperazin-1- yl)methyl)-4-(trifluoromethyl)isoindolin- 1-one |
| 279 | 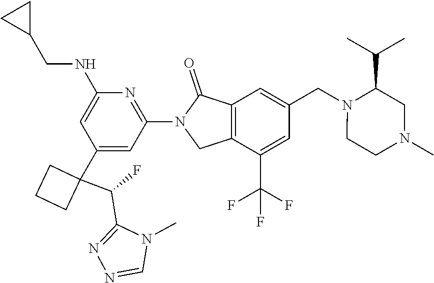 [SMILES: CC(C)[C@H]1CN(C)CCN1Cc1cc2c(c(C(F)(F)F)c1)CN(c1cc(C3([C@H](F)c4nncn4C)CCC3)cc(NCC3CC3)n1)C2=O] | 2-(6-((cyclopropylmethyl)amino)-4-(1- ((S)-fluoro(4-methyl-4H-1,2,4-triazol-3- yl)methyl)cyclobutyl)pyridin-2-yl)-6- (((S)-2-isopropyl-4-methylpiperazin-1- yl)methyl)-4-(trifluoromethyl)isoindolin- 1-one |
| 280 | 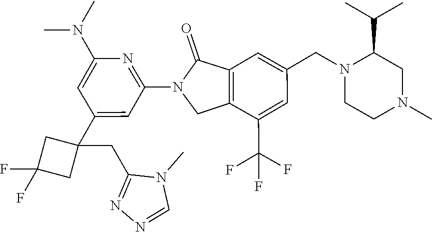 [SMILES: CC(C)[C@H]1CN(C)CCN1Cc1cc2c(c(C(F)(F)F)c1)CN(c1cc(C3(Cc4nncn4C)CC(F)(F)C3)cc(N(C)C)n1)C2=O] | (S)-2-(4-(3,3-difluoro-1-((4-methyl-4H- 1,2,4-triazol-3-yl)methyl)cyclobutyl)-6- (dimethylamino)pyridin-2-yl)-6-((2- isopropyl-4-methylpiperazin-1- yl)methyl)-4-(trifluoromethyl)isoindolin- 1-one |
| 281 | 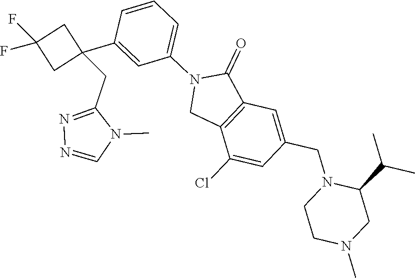 [SMILES: CC(C)[C@H]1CN(C)CCN1Cc1cc(Cl)c2c(c1)C(=O)N(c1cccc(C3(Cc4nncn4C)CC(F)(F)C3)c1)C2] | (S)-4-chloro-2-(3-(3,3-difluoro-1-((4- methyl-4H-1,2,4-triazol-3- yl)methyl)cyclobutyl)phenyl)-6-((2- isopropyl-4-methylpiperazin-1- yl)methyl)isoindolin-1-one |
| 282 | 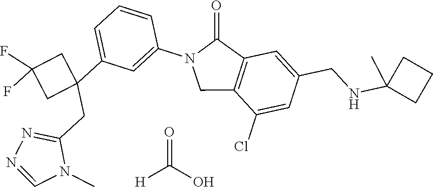 | 4-chloro-2-(3-(3,3-difluoro-1-((4-methyl- 4H-1,2,4-triazol-3- yl)methyl)cyclobutyl)phenyl)-6-(((1- methylcyclobutyl)amino)methyl) isoindolin-1-one formate |
| 283 | 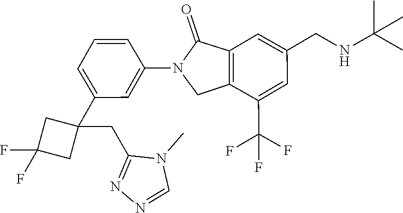 [SMILES: Cn1cnnc1CC1(c2cccc(N3Cc4c(cc(CNC(C)(C)C)cc4C(F)(F)F)C3=O)c2)CC(F)(F)C1] | 6-((tert-butylamino)methyl)-2-(3-(3,3- difluoro-1-((4-methyl-4H-1,2,4-triazol-3- yl)methyl)cyclobutyl)phenyl)-4- (trifluoromethyl)isoindolin-1-one |
| 284 | 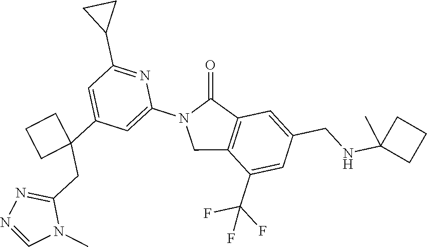 [SMILES: Cn1cnnc1CC1(c2cc(C3CC3)nc(N3Cc4c(cc(CNC5(C)CCC5)cc4C(F)(F)F)C3=O)c2)CCC1] | 2-(6-cyclopropyl-4-(1-((4-methyl-4H- 1,2,4-triazol-3- yl)methyl)cyclobutyl)pyridin-2-yl)-6-(((1- methylcyclobutyl)amino)methyl)-4- (trifluoromethyl)isoindolin-1-one |
| 285 | 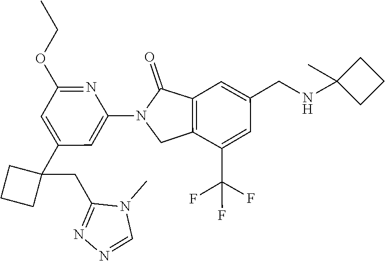 [SMILES: CCOc1cc(C2(Cc3nncn3C)CCC2)cc(N2Cc3c(cc(CNC4(C)CCC4)cc3C(F)(F)F)C2=O)n1] | 2-(6-ethoxy-4-(1-((4-methyl-4H-1,2,4- triazol-3-yl)methyl)cyclobutyl)pyridin-2- yl)-6-(((1- methylcyclobutyl)amino)methyl)-4- (trifluoromethyl)isoindolin-1-one |
| 286 | 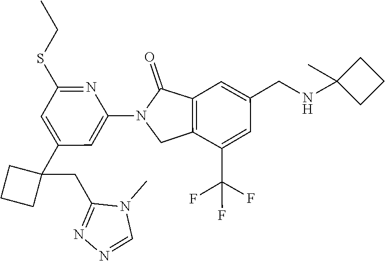 [SMILES: CCSc1cc(C2(Cc3nncn3C)CCC2)cc(N2Cc3c(cc(CNC4(C)CCC4)cc3C(F)(F)F)C2=O)n1] | 2-(6-(ethylthio)-4-(1-((4-methyl-4H- 1,2,4-triazol-3- yl)methyl)cyclobutyl)pyridin-2-yl)-6-(((1- methylcyclobutyl)amino)methyl)-4- (trifluoromethyl)isoindolin-1-one |
| 287 | 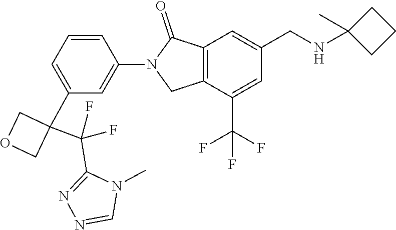 [SMILES: Cn1cnnc1C(F)(F)C1(c2cccc(N3Cc4c(cc(CNC5(C)CCC5)cc4C(F)(F)F)C3=O)c2)COC1] | 2-(3-(3-(difluoro(4-methyl-4H-1,2,4- triazol-3-yl)methyl)oxetan-3-yl)phenyl)- 6-(((1-methylcyclobutyl)amino)methyl)- 4-(trifluoromethyl)isoindolin-1-one |
| 288 | 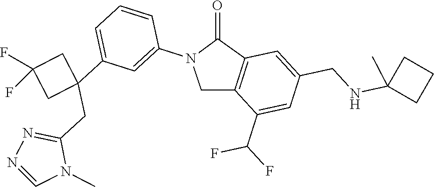 [SMILES: Cn1cnnc1CC1(c2cccc(N3Cc4c(cc(CNC5(C)CCC5)cc4C(F)F)C3=O)c2)CC(F)(F)C1] | 2-(3-(3,3-difluoro-1-((4-methyl-4H- 1,2,4-triazol-3- yl)methyl)cyclobutyl)phenyl)-4- (difluoromethyl)-6-(((1- methylcyclobutyl)amino)methyl) isoindolin-1-one |
| 289 | 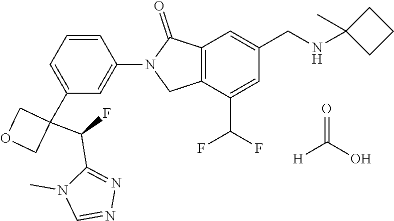 | (R)-4-(difluoromethyl)-2-(3-(3-(fluoro(4- methyl-4H-1,2,4-triazol-3- yl)methyl)oxetan-3-yl)phenyl)-6-(((1- methylcyclobutyl)amino)methyl)? isoindolin-1-one formate |
| 290 | 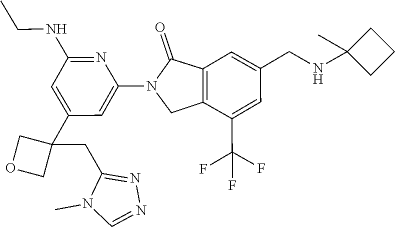 [SMILES: CCNc1cc(C2(Cc3nncn3C)COC2)cc(N2Cc3c(cc(CNC4(C)CCC4)cc3C(F)(F)F)C2=O)n1] | 2-(6-(ethylamino)-4-(3-((4-methyl-4H- 1,2,4-triazol-3-yl)methyl)oxetan-3- yl)pyridin-2-yl)-6-(((1- methylcyclobutyl)amino)methyl)-4- (trifluoromethyl)isoindolin-1-one |
| 291 | 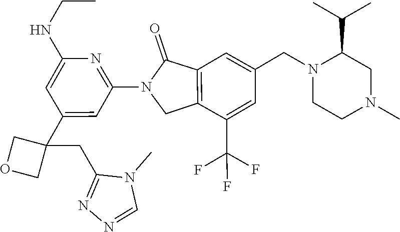 [SMILES: CCNc1cc(C2(Cc3nncn3C)COC2)cc(N2Cc3c(cc(CN4CCN(C)C[C@@H]4C(C)C)cc3C(F)(F)F)C2=O)n1] | (S)-2-(6-(ethylamino)-4-(3-((4-methyl- 4H-1,2,4-triazol-3-yl)methyl)oxetan-3- yl)pyridin-2-yl)-6-((2-isopropyl-4- methylpiperazin-1-yl)methyl)-4- (trifluoromethyl)isoindolin-1-one |
| 292 | 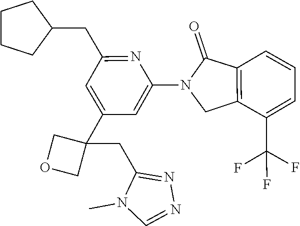 [SMILES: Cn1cnnc1CC1(c2cc(CC3CCCC3)nc(N3Cc4c(cccc4C(F)(F)F)C3=O)c2)COC1] | 2-(6-(cyclopentylmethyl)-4-(3-((4- methyl-4H-1,2,4-triazol-3- yl)methyl)oxetan-3-yl)pyridin-2-yl)-4- (trifluoromethyl)isoindolin-1-one |
| 293 | 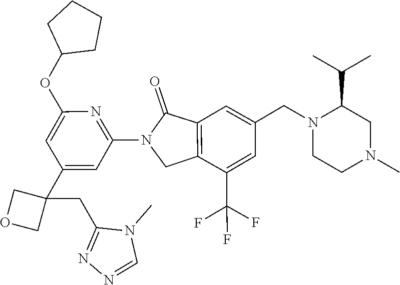 [SMILES: CC(C)[C@H]1CN(C)CCN1Cc1cc2c(c(C(F)(F)F)c1)CN(c1cc(C3(Cc4nncn4C)COC3)cc(OC3CCCC3)n1)C2=O] | (S)-2-(6-(cyclopentyloxy)-4-(3-((4- methyl-4H-1,2,4-triazol-3- yl)methyl)oxetan-3-yl)pyridin-2-yl)-6- ((2-isopropyl-4-methylpiperazin-1- yl)methyl)-4-(trifluoromethyl)isoindolin- 1-one |
| 294 | 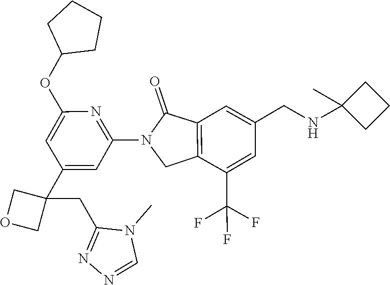 [SMILES: Cn1cnnc1CC1(c2cc(OC3CCCC3)nc(N3Cc4c(cc(CNC5(C)CCC5)cc4C(F)(F)F)C3=O)c2)COC1] | 2-(6-(cyclopentyloxy)-4-(3-((4-methyl- 4H-1,2,4-triazol-3-yl)methyl)oxetan-3- yl)pyridin-2-yl)-6-(((1- methylcyclobutyl)amino)methyl)-4- (trifluoromethyl)isoindolin-1-one |
| 295 | 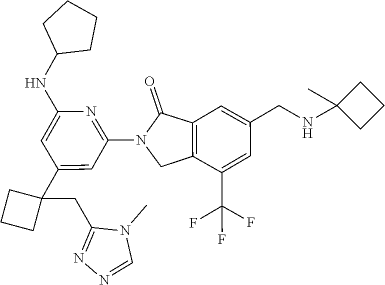 [SMILES: Cn1cnnc1CC1(c2cc(NC3CCCC3)nc(N3Cc4c(cc(CNC5(C)CCC5)cc4C(F)(F)F)C3=O)c2)CCC1] | 2-(6-(cyclopentylamino)-4-(1-((4- methyl-4H-1,2,4-triazol-3- yl)methyl)cyclobutyl)pyridin-2-yl)-6-(((1- methylcyclobutyl)amino)methyl)-4- (trifluoromethyl)isoindolin-1-one |
| 296 | 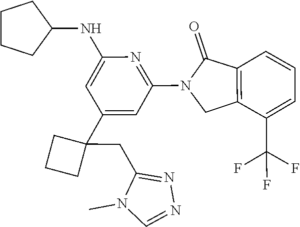 [SMILES: Cn1cnnc1CC1(c2cc(NC3CCCC3)nc(N3Cc4c(cccc4C(F)(F)F)C3=O)c2)CCC1] | 2-(6-(cyclopentylamino)-4-(1-((4- methyl-4H-1,2,4-triazol-3- yl)methyl)cyclobutyl)pyridin-2-yl)-4- (trifluoromethyl)isoindolin-1-one |
| 297 | 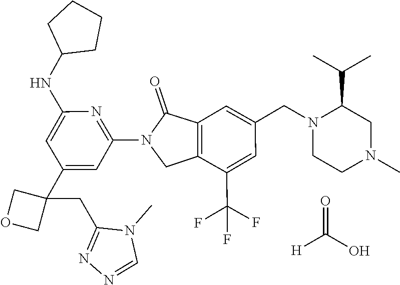 | (S)-2-(6-(cyclopentylamino)-4-(3-((4- methyl-4H-1,2,4-triazol-3- yl)methyl)oxetan-3-yl)pyridin-2-yl)-6- ((2-isopropyl-4-methylpiperazin-1- yl)methyl)-4-(trifluoromethyl)isoindolin- 1-one formate |
| 298 | 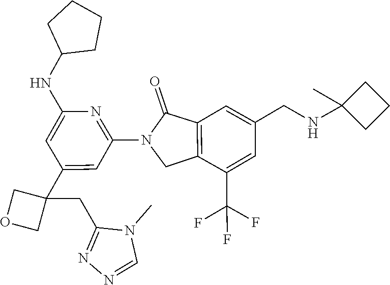 [SMILES: Cn1cnnc1CC1(c2cc(NC3CCCC3)nc(N3Cc4c(cc(CNC5(C)CCC5)cc4C(F)(F)F)C3=O)c2)COC1] | 2-(6-(cyclopentylamino)-4-(3-((4- methyl-4H-1,2,4-triazol-3- yl)methyl)oxetan-3-yl)pyridin-2-yl)-6- (((1-methylcyclobutyl)amino)methyl)-4- (trifluoromethyl)isoindolin-1-one |
| 299 | 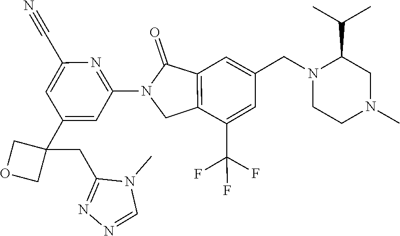 [SMILES: CC(C)[C@H]1CN(C)CCN1Cc1cc2c(c(C(F)(F)F)c1)CN(c1cc(C3(Cc4nncn4C)COC3)cc(C#N)n1)C2=O] | (S)-6-(6-((2-isopropyl-4- methylpiperazin-1-yl)methyl)-1-oxo-4- (trifluoromethyl)isoindolin-2-yl)-4-(1-((4- methyl-4H-1,2,4-triazol-3- yl)methyl)cyclobutyl)picolinonitrile |
| 300 | 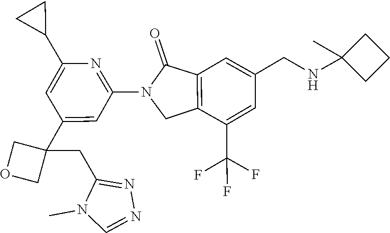 [SMILES: Cn1cnnc1CC1(c2cc(C3CC3)nc(N3Cc4c(cc(CNC5(C)CCC5)cc4C(F)(F)F)C3=O)c2)COC1] | 2-(6-cyclopropyl-4-(3-((4-methyl-4H- 1,2,4-triazol-3-yl)methyl)oxetan-3- yl)pyridin-2-yl)-6-(((1- methylcyclobutyl)amino)methyl)-4- (trifluoromethyl)isoindolin-1-one |
| 301 | 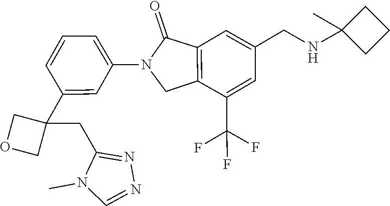 [SMILES: Cn1cnnc1CC1(c2cccc(N3Cc4c(cc(CNC5(C)CCC5)cc4C(F)(F)F)C3=O)c2)COC1] | 2-(3-(3-((4-methyl-4H-1,2,4-triazol-3- yl)methyl)oxetan-3-yl)phenyl)-6-(((1- methylcyclobutyl)amino)methyl)-4- (trifluoromethyl)isoindolin-1-one |
| 302 | 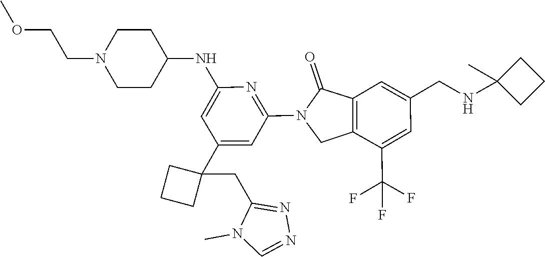 [SMILES: COCCN1CCC(Nc2cc(C3(Cc4nncn4C)CCC3)cc(N3Cc4c(cc(CNC5(C)CCC5)cc4C(F)(F)F)C3=O)n2)CC1] | 2-(6-((1-(2-methoxyethyl)piperidin-4- yl)amino)-4-(1-((4-methyl-4H-1,2,4- triazol-3-yl)methyl)cyclobutyl)pyridin-2- yl)-6-(((1- methylcyclobutyl)amino)methyl)-4- (trifluoromethyl)isoindolin-1-one |
| 303 | 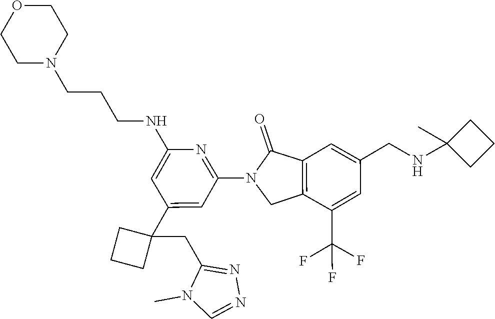 [SMILES: Cn1cnnc1CC1(c2cc(NCCCN3CCOCC3)nc(N3Cc4c(cc(CNC5(C)CCC5)cc4C(F)(F)F)C3=O)c2)CCC1] | 2-(4-(1-((4-methyl-4H-1,2,4-triazol-3- yl)methyl)cyclobutyl)-6-((3- morpholinopropyl)amino)pyridin-2-yl)- 6-(((1-methylcyclobutyl)amino)methyl)- 4-(trifluoromethyl)isoindolin-1-one |
| 304 | 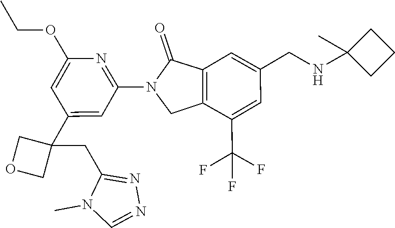 [SMILES: CCOc1cc(C2(Cc3nncn3C)COC2)cc(N2Cc3c(cc(CNC4(C)CCC4)cc3C(F)(F)F)C2=O)n1] | 2-(6-ethoxy-4-(3-((4-methyl-4H-1,2,4- triazol-3-yl)methyl)oxetan-3-yl)pyridin- 2-yl)-6-(((1- methylcyclobutyl)amino)methyl)-4- (trifluoromethyl)isoindolin-1-one |
| 305 | 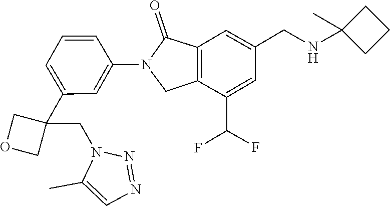 [SMILES: Cc1cnnn1CC1(c2cccc(N3Cc4c(cc(CNC5(C)CCC5)cc4C(F)F)C3=O)c2)COC1] | 4-(difluoromethyl)-2-(3-(3-((5-methyl- 1H-1,2,3-triazol-1-yl)methyl)oxetan-3- yl)phenyl)-6-(((1- methylcyclobutyl)amino)methyl) isoindolin-1-one |
| 306 | 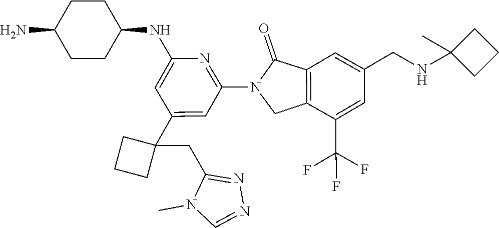 [SMILES: Cn1cnnc1CC1(c2cc(N[C@H]3CC[C@@H](N)CC3)nc(N3Cc4c(cc(CNC5(C)CCC5)cc4C(F)(F)F)C3=O)c2)CCC1] | 2-(6-(((1s,4s)-4- aminocyclohexyl)amino)-4-(1-((4- methyl-4H-1,2,4-triazol-3- yl)methyl)cyclobutyl)pyridin-2-yl)-6-(((1- methylcyclobutyl)amino)methyl)-4- (trifluoromethyl)isoindolin-1-one |
| 307 | 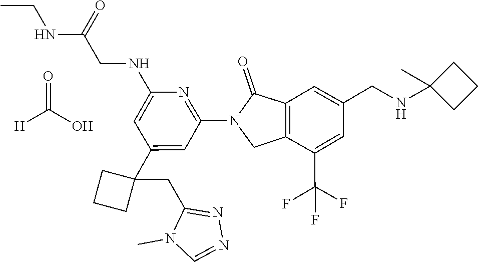 | N-ethyl-2-((4-(1-((4-methyl-4H-1,2,4- triazol-3-yl)methyl)cyclobutyl)-6-(6-(((1- methylcyclobutyl)amino)methyl)-1-oxo- 4-(trifluoromethyl)isoindolin-2- yl)pyridin-2-yl)amino)acetamide formate |
| 308 | 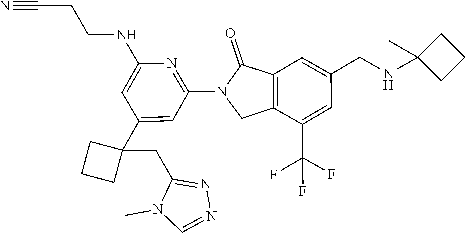 [SMILES: Cn1cnnc1CC1(c2cc(NCCC#N)nc(N3Cc4c(cc(CNC5(C)CCC5)cc4C(F)(F)F)C3=O)c2)CCC1] | 3-((4-(1-((4-methyl-4H-1,2,4-triazol-3- yl)methyl)cyclobutyl)-6-(6-(((1- methylcyclobutyl)amino)methyl)-1-oxo- 4-(trifluoromethyl)isoindolin-2- yl)pyridin-2-yl)amino)propanenitrile |
| 309 | 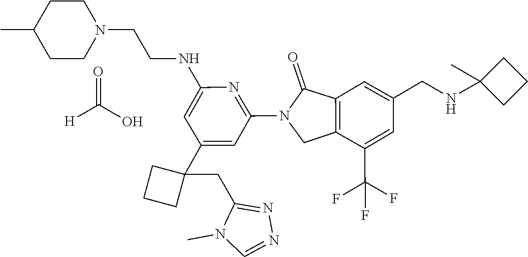 | 2-(4-(1-((4-methyl-4H-1,2,4-triazol-3- yl)methyl)cyclobutyl)-6-((2-(4- methylpiperidin-1- yl)ethyl)amino)pyridin-2-yl)-6-(((1- methylcyclobutyl)amino)methyl)-4- (trifluoromethyl)isoindolin-1-one formate |
| 310 | 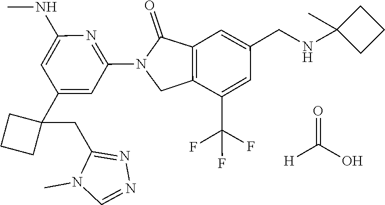 | 2-(4-(1-((4-methyl-4H-1,2,4-triazol-3- yl)methyl)cyclobutyl)-6- (methylamino)pyridin-2-yl)-6-(((1- methylcyclobutyl)amino)methyl)-4- (trifluoromethyl)isoindolin-1-one formate |
| 311 | 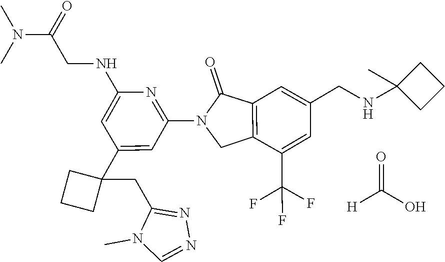 | N,N-dimethyl-2-((4-(1-((4-methyl-4H- 1,2,4-triazol-3-yl)methyl)cyclobutyl)-6- (6-(((1-methylcyclobutyl)amino)methyl)- 1-oxo-4-(trifluoromethyl)isoindolin-2- yl)pyridin-2-yl)amino)acetamide formate |
| 312 | 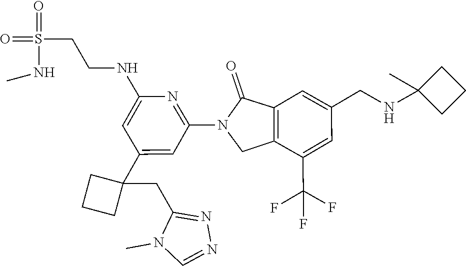 [SMILES: CNS(=O)(=O)CCNc1cc(C2(Cc3nncn3C)CCC2)cc(N2Cc3c(cc(CNC4(C)CCC4)cc3C(F)(F)F)C2=O)n1] | N-methyl-2-((4-(1-((4-methyl-4H-1,2,4- triazol-3-yl)methyl)cyclobutyl)-6-(6-(((1- methylcyclobutyl)amino)methyl)-1-oxo- 4-(trifluoromethyl)isoindolin-2- yl)pyridin-2- yl)amino)ethanesulfonamide |
| 313 | 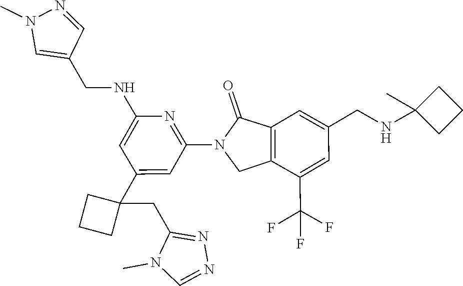 [SMILES: Cn1cc(CNc2cc(C3(Cc4nncn4C)CCC3)cc(N3Cc4c(cc(CNC5(C)CCC5)cc4C(F)(F)F)C3=O)n2)cn1] | 2-(6-(((1-methyl-1H-pyrazol-4- yl)methyl)amino)-4-(1-((4-methyl-4H- 1,2,4-triazol-3- yl)methyl)cyclobutyl)pyridin-2-yl)-6-(((1- methylcyclobutyl)amino)methyl)-4- (trifluoromethyl)isoindolin-1-one |
| 314 | 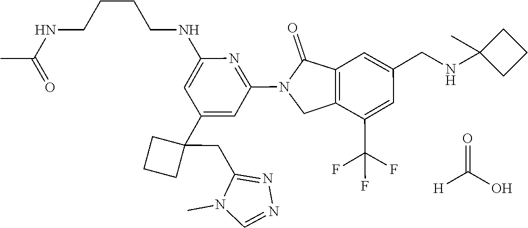 [SMILES: CC(=O)NCCCCNc1cc(C2(Cc3nncn3C)CCC2)cc(N2Cc3c(cc(CNC4(C)CCC4)cc3C(F)(F)F)C2=O)n1 ; O=CO] | N-(4-((4-(1-((4-methyl-4H-1,2,4-triazol- 3-yl)methyl)cyclobutyl)-6-(6-(((1- methylcyclobutyl)amino)methyl)-1-oxo- 4-(trifluoromethyl)isoindolin-2- yl)pyridin-2-yl)amino)butyl)acetamide formate |
| 315 | 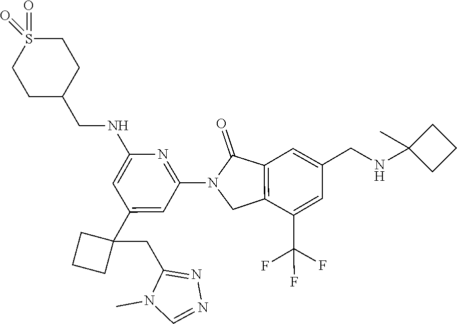 [SMILES: Cn1cnnc1CC1(c2cc(NCC3CCS(=O)(=O)CC3)nc(N3Cc4c(cc(CNC5(C)CCC5)cc4C(F)(F)F)C3=O)c2)CCC1] | 2-(6-(((1,1-dioxidotetrahydro-2H- thiopyran-4-yl)methyl)amino)-4-(1-((4- methyl-4H-1,2,4-triazol-3- yl)methyl)cyclobutyl)pyridin-2-yl)-6-(((1- methylcyclobutyl)amino)methyl)-4- (trifluoromethyl)isoindolin-1-one |
| 316 | 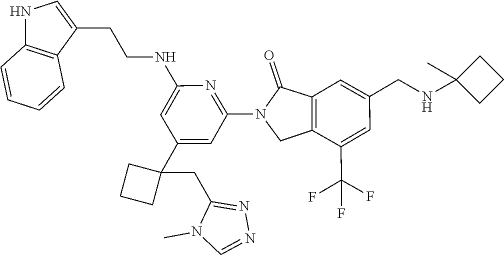 [SMILES: Cn1cnnc1CC1(c2cc(NCCc3c[nH]c4ccccc34)nc(N3Cc4c(cc(CNC5(C)CCC5)cc4C(F)(F)F)C3=O)c2)CCC1] | 2-(6-((2-(1H-indol-3-yl)ethyl)amino)-4- (1-((4-methyl-4H-1,2,4-triazol-3- yl)methyl)cyclobutyl)pyridin-2-yl)-6-(((1- methylcyclobutyl)amino)methyl)-4- (trifluoromethyl)isoindolin-1-one |
| 317 | 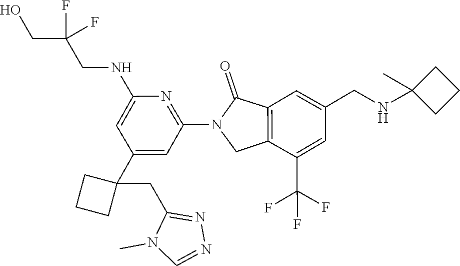 [SMILES: Cn1cnnc1CC1(c2cc(NCC(F)(F)CO)nc(N3Cc4c(cc(CNC5(C)CCC5)cc4C(F)(F)F)C3=O)c2)CCC1] | 2-(6-((2,2-difluoro-3- hydroxypropyl)amino)-4-(1-((4-methyl- 4H-1,2,4-triazol-3- yl)methyl)cyclobutyl)pyridin-2-yl)-6-(((1- methylcyclobutyl)amino)methyl)-4- (trifluoromethyl)isoindolin-1-one |
| 318 | 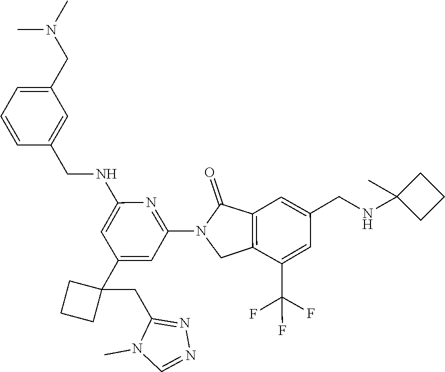 [SMILES: CN(C)Cc1cccc(CNc2cc(C3(Cc4nncn4C)CCC3)cc(N3Cc4c(cc(CNC5(C)CCC5)cc4C(F)(F)F)C3=O)n2)c1] | 2-(6-((3- ((dimethylamino)methyl)benzyl)amino)- 4-(1-((4-methyl-4H-1,2,4-triazol-3- yl)methyl)cyclobutyl)pyridin-2-yl)-6-(((1- methylcyclobutyl)amino)methyl)-4- (trifluoromethyl)isoindolin-1-one |
| 319 | 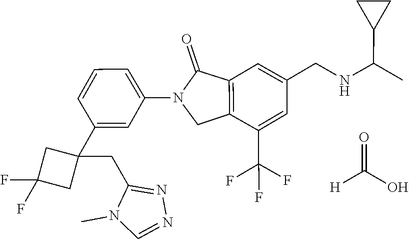 [SMILES: CC(NCc1cc2c(c(C(F)(F)F)c1)CN(c1cccc(C3(Cc4nncn4C)CC(F)(F)C3)c1)C2=O)C1CC1 ; O=CO] | 6-(((1-cyclopropylethyl)amino)methyl)- 2-(3-(3,3-difluoro-1-((4-methyl-4H- 1,2,4-triazol-3- yl)methyl)cyclobutyl)phenyl)-4- (trifluoromethyl)isoindolin-1-one formate |
| 320 | 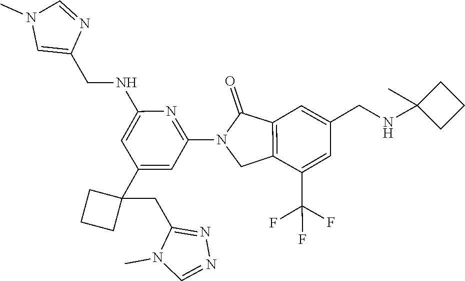 [SMILES: Cn1cnc(CNc2cc(C3(Cc4nncn4C)CCC3)cc(N3Cc4c(cc(CNC5(C)CCC5)cc4C(F)(F)F)C3=O)n2)c1] | 2-(6-(((1-methyl-1H-imidazol-4- yl)methyl)amino)-4-(1-((4-methyl-4H- 1,2,4-triazol-3- yl)methyl)cyclobutyl)pyridin-2-yl)-6-(((1- methylcyclobutyl)amino)methyl)-4- (trifluoromethyl)isoindolin-1-one |
| 321 | 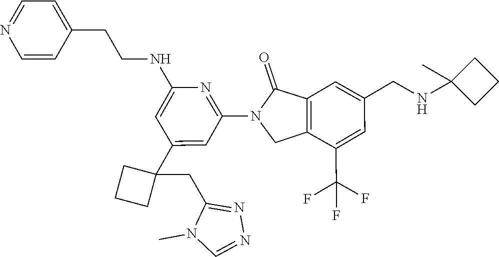 [SMILES: Cn1cnnc1CC1(c2cc(NCCc3ccncc3)nc(N3Cc4c(cc(CNC5(C)CCC5)cc4C(F)(F)F)C3=O)c2)CCC1] | 2-(4-(1-((4-methyl-4H-1,2,4-triazol-3- yl)methyl)cyclobutyl)-6-((2-(pyridin-4- yl)ethyl)amino)pyridin-2-yl)-6-(((1- methylcyclobutyl)amino)methyl)-4- (trifluoromethyl)isoindolin-1-one |
| 322 | 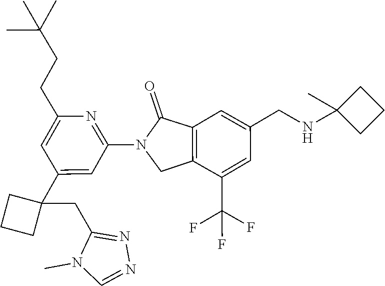 [SMILES: Cn1cnnc1CC1(c2cc(CCC(C)(C)C)nc(N3Cc4c(cc(CNC5(C)CCC5)cc4C(F)(F)F)C3=O)c2)CCC1] | 2-(6-(3,3-dimethylbutyl)-4-(1-((4- methyl-4H-1,2,4-triazol-3- yl)methyl)cyclobutyl)pyridin-2-yl)-6-(((1- methylcyclobutyl)amino)methyl)-4- (trifluoromethyl)isoindolin-1-one |
| 323 | 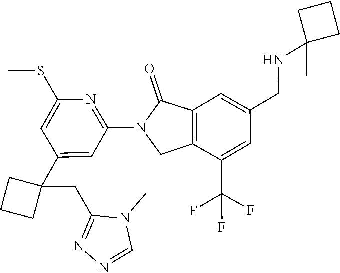 [SMILES: CSc1cc(C2(Cc3nncn3C)CCC2)cc(N2Cc3c(cc(CNC4(C)CCC4)cc3C(F)(F)F)C2=O)n1] | 2-(4-(1-((4-methyl-4H-1,2,4-triazol-3- yl)methyl)cyclobutyl)-6- (methylthio)pyridin-2-yl)-6-(((1- methylcyclobutyl)amino)methyl)-4- (trifluoromethyl)isoindolin-1-one |
| 324 | 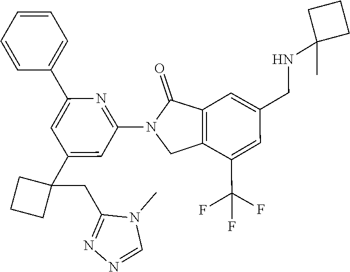 [SMILES: Cn1cnnc1CC1(c2cc(-c3ccccc3)nc(N3Cc4c(cc(CNC5(C)CCC5)cc4C(F)(F)F)C3=O)c2)CCC1] | 2-(4-(1-((4-methyl-4H-1,2,4-triazol-3- yl)methyl)cyclobutyl)-6-phenylpyridin-2- yl)-6-(((1- methylcyclobutyl)amino)methyl)-4- (trifluoromethyl)isoindolin-1-one |
| 325 | 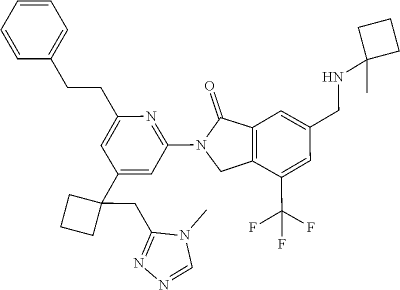 [SMILES: Cn1cnnc1CC1(c2cc(CCc3ccccc3)nc(N3Cc4c(cc(CNC5(C)CCC5)cc4C(F)(F)F)C3=O)c2)CCC1] | 2-(4-(1-((4-methyl-4H-1,2,4-triazol-3- yl)methyl)cyclobutyl)-6- phenethylpyridin-2-yl)-6-(((1- methylcyclobutyl)amino)methyl)-4- (trifluoromethyl)isoindolin-1-one |
| 326 | 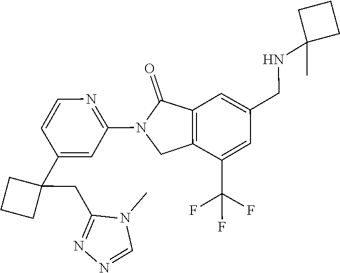 [SMILES: Cn1cnnc1CC1(c2ccnc(N3Cc4c(cc(CNC5(C)CCC5)cc4C(F)(F)F)C3=O)c2)CCC1] | 2-(4-(1-((4-methyl-4H-1,2,4-triazol-3- yl)methyl)cyclobutyl)pyridin-2-yl)-6-(((1- methylcyclobutyl)amino)methyl)-4- (trifluoromethyl)isoindolin-1-one |
| 327 | 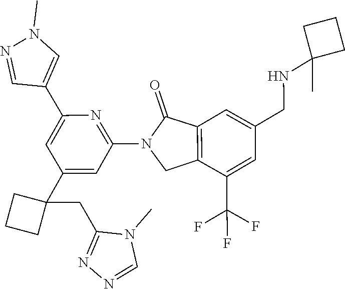 [SMILES: Cn1cc(-c2cc(C3(Cc4nncn4C)CCC3)cc(N3Cc4c(cc(CNC5(C)CCC5)cc4C(F)(F)F)C3=O)n2)cn1] | 2-(6-(1-methyl-1H-pyrazol-4-yl)-4-(1- ((4-methyl-4H-1,2,4-triazol-3- yl)methyl)cyclobutyl)pyridin-2-yl)-6-(((1- methylcyclobutyl)amino)methyl)-4- (trifluoromethyl)isoindolin-1-one |
| 328 | 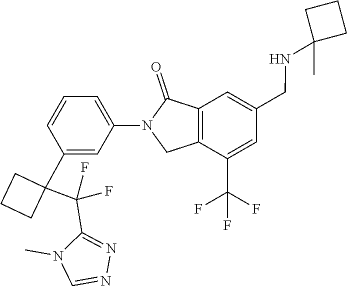 [SMILES: Cn1cnnc1C(F)(F)C1(c2cccc(N3Cc4c(cc(CNC5(C)CCC5)cc4C(F)(F)F)C3=O)c2)CCC1] | 2-(3-(1-(difluoro(4-methyl-4H-1,2,4- triazol-3-yl)methyl)cyclobutyl)phenyl)-6- (((1-methylcyclobutyl)amino)methyl)-4- (trifluoromethyl)isoindolin-1-one |
| 329 | 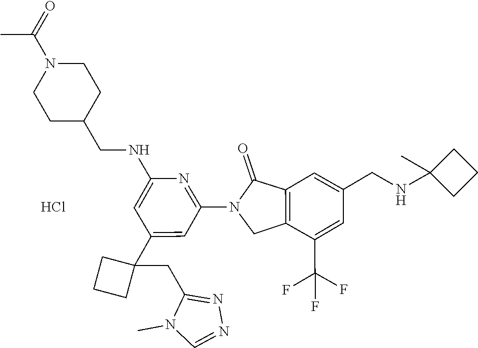 [SMILES: CC(=O)N1CCC(CNc2cc(C3(Cc4nncn4C)CCC3)cc(N3Cc4c(cc(CNC5(C)CCC5)cc4C(F)(F)F)C3=O)n2)CC1 ; Cl] | 2-(6-(((1-acetylpiperidin-4- yl)methyl)amino)-4-(1-((4-methyl-4H- 1,2,4-triazol-3- yl)methyl)cyclobutyl)pyridin-2-yl)-6-(((1- methylcyclobutyl)amino)methyl)-4- (trifluoromethyl)isoindolin-1-one hydrochloride |
| 330 | 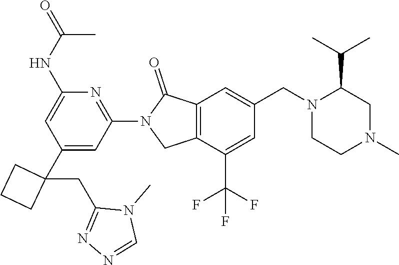 [SMILES: CC(=O)Nc1cc(C2(Cc3nncn3C)CCC2)cc(N2Cc3c(cc(CN4CCN(C)C[C@@H]4C(C)C)cc3C(F)(F)F)C2=O)n1] | (S)-N-(6-(6-((2-isopropyl-4- methylpiperazin-1-yl)methyl)-1-oxo-4- (trifluoromethyl)isoindolin-2-yl)-4-(1-((4- methyl-4H-1,2,4-triazol-3- yl)methyl)cyclobutyl)pyridin-2- yl)acetamide |
| 331 | 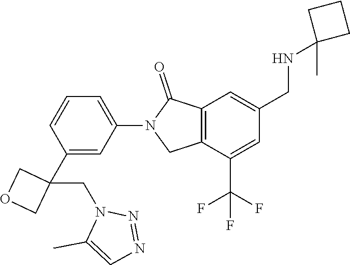 [SMILES: Cc1cnnn1CC1(c2cccc(N3Cc4c(cc(CNC5(C)CCC5)cc4C(F)(F)F)C3=O)c2)COC1] | 2-(3-(3-((4-methyl-4H-1,2,4-triazol-3- yl)methyl)oxetan-3-yl)phenyl)-6-(((1- methylcyclobutyl)amino)methyl)-4- (trifluoromethyl)isoindolin-1-one |
| 332 | 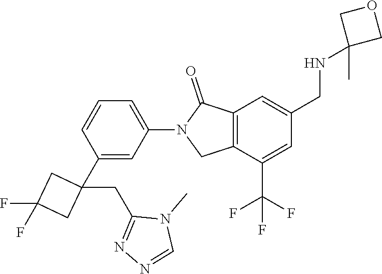 [SMILES: Cn1cnnc1CC1(c2cccc(N3Cc4c(cc(CNC5(C)COC5)cc4C(F)(F)F)C3=O)c2)CC(F)(F)C1] | 2-(3-(3,3-difluoro-1-((4-methyl-4H- 1,2,4-triazol-3- yl)methyl)cyclobutyl)phenyl)-6-(((3- methyloxetan-3-yl)amino)methyl)-4- (trifluoromethyl)isoindolin-1-one |
| 333 | 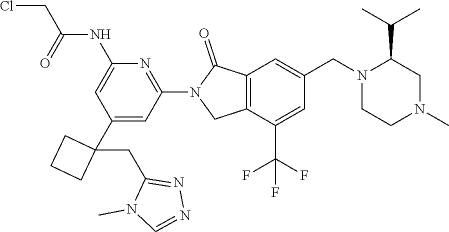 [SMILES: CC(C)[C@H]1CN(C)CCN1Cc1cc2c(c(C(F)(F)F)c1)CN(c1cc(C3(Cc4nncn4C)CCC3)cc(NC(=O)CCl)n1)C2=O] | (S)-2-chloro-N-(6-(6-((2-isopropyl-4- methylpiperazin-1-yl)methyl)-1-oxo-4- (trifluoromethyl)isoindolin-2-yl)-4-(1-((4- methyl-4H-1,2,4-triazol-3- yl)methyl)cyclobutyl)pyridin-2- yl)acetamide |
| 334 | 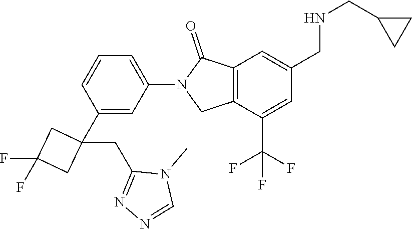 [SMILES: Cn1cnnc1CC1(c2cccc(N3Cc4c(cc(CNCC5CC5)cc4C(F)(F)F)C3=O)c2)CC(F)(F)C1] | 6-(((cyclopropylmethyl)amino)methyl)- 2-(3-(3,3-difluoro-1-((4-methyl-4H- 1,2,4-triazol-3- yl)methyl)cyclobutyl)phenyl)-4- (trifluoromethyl)isoindolin-1-one |
| 335 | 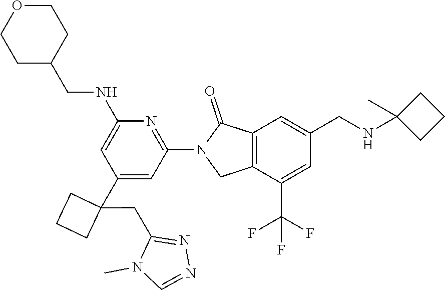 [SMILES: Cn1cnnc1CC1(c2cc(NCC3CCOCC3)nc(N3Cc4c(cc(CNC5(C)CCC5)cc4C(F)(F)F)C3=O)c2)CCC1] | 2-(4-(1-((4-methyl-4H-1,2,4-triazol-3- yl)methyl)cyclobutyl)-6-(((tetrahydro- 2H-pyran-4-yl)methyl)amino)pyridin-2- yl)-6-(((1- methylcyclobutyl)amino)methyl)-4- (trifluoromethyl)isoindolin-1-one |
| 336 | 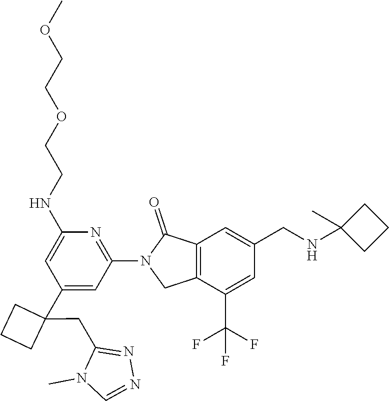 [SMILES: COCCOCCNc1cc(C2(Cc3nncn3C)CCC2)cc(N2Cc3c(cc(CNC4(C)CCC4)cc3C(F)(F)F)C2=O)n1] | 2-(6-((2-(2- methoxyethoxy)ethyl)amino)-4-(1-((4- methyl-4H-1,2,4-triazol-3- yl)methyl)cyclobutyl)pyridin-2-yl)-6-(((1- methylcyclobutyl)amino)methyl)-4- (trifluoromethyl)isoindolin-1-one |
| 337 | 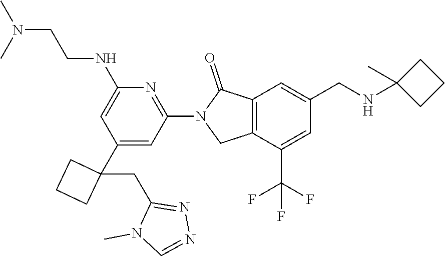 [SMILES: CN(C)CCNc1cc(C2(Cc3nncn3C)CCC2)cc(N2Cc3c(cc(CNC4(C)CCC4)cc3C(F)(F)F)C2=O)n1] | 2-(6-((2-(dimethylamino)ethyl)amino)- 4-(1-((4-methyl-4H-1,2,4-triazol-3- yl)methyl)cyclobutyl)pyridin-2-yl)-6-(((1- methylcyclobutyl)amino)methyl)-4- (trifluoromethyl)isoindolin-1-one |
| 338 | 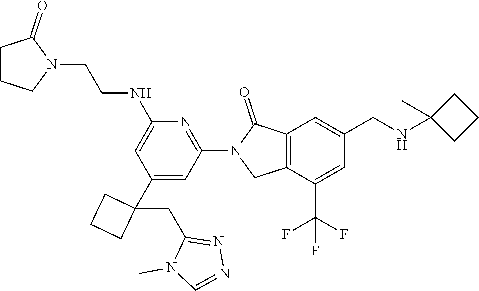 [SMILES: Cn1cnnc1CC1(c2cc(NCCN3CCCC3=O)nc(N3Cc4c(cc(CNC5(C)CCC5)cc4C(F)(F)F)C3=O)c2)CCC1] | 2-(4-(1-((4-methyl-4H-1,2,4-triazol-3- yl)methyl)cyclobutyl)-6-((2-(2- oxopyrrolidin-1-yl)ethyl)amino)pyridin- 2-yl)-6-(((1- methylcyclobutyl)amino)methyl)-4- (trifluoromethyl)isoindolin-1-one |
| 339 | 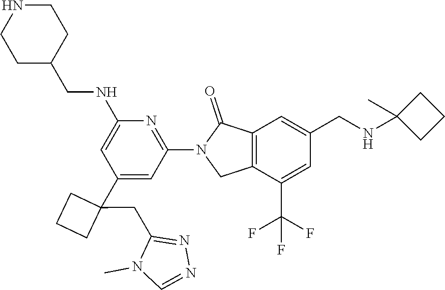 [SMILES: Cn1cnnc1CC1(c2cc(NCC3CCNCC3)nc(N3Cc4c(cc(CNC5(C)CCC5)cc4C(F)(F)F)C3=O)c2)CCC1] | 2-(4-(1-((4-methyl-4H-1,2,4-triazol-3- yl)methyl)cyclobutyl)-6-((piperidin-4- ylmethyl)amino)pyridin-2-yl)-6-(((1- methylcyclobutyl)amino)methyl)-4- (trifluoromethyl)isoindolin-1-one |
| 340 | 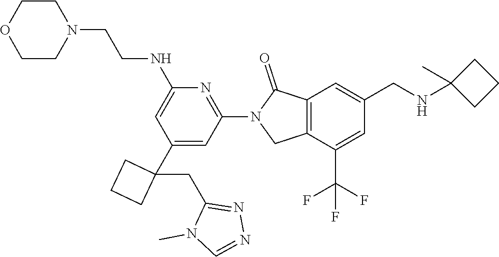 [SMILES: Cn1cnnc1CC1(c2cc(NCCN3CCOCC3)nc(N3Cc4c(cc(CNC5(C)CCC5)cc4C(F)(F)F)C3=O)c2)CCC1] | 2-(4-(1-((4-methyl-4H-1,2,4-triazol-3- yl)methyl)cyclobutyl)-6-((2- morpholinoethyl)amino)pyridin-2-yl)-6- (((1-methylcyclobutyl)amino)methyl)-4- (trifluoromethyl)isoindolin-1-one |
| 341 | 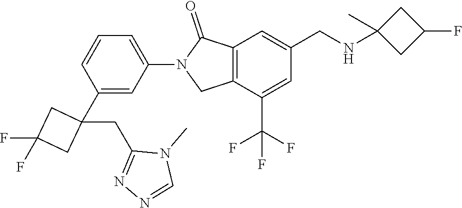 [SMILES: Cn1cnnc1CC1(c2cccc(N3Cc4c(cc(CNC5(C)CC(F)C5)cc4C(F)(F)F)C3=O)c2)CC(F)(F)C1] | 2-(3-(3,3-difluoro-1-((4-methyl-4H- 1,2,4-triazol-3- yl)methyl)cyclobutyl)phenyl)-6-(((3- fluoro-1- methylcyclobutyl)amino)methyl)-4- (trifluoromethyl)isoindolin-1-one |
| 342 | 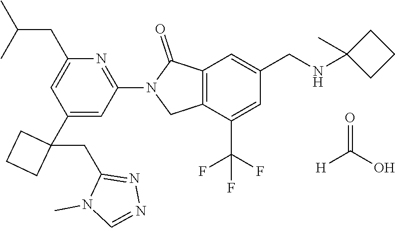 | 2-(6-isobutyl-4-(1-((4-methyl-4H-1,2,4- triazol-3-yl)methyl)cyclobutyl)pyridin-2- yl)-6-(((1- methylcyclobutyl)amino)methyl)-4- (trifluoromethyl)isoindolin-1-one formate |
| 343 | 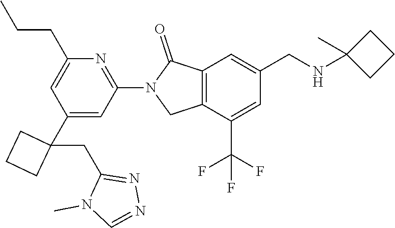 [SMILES: CCCc1cc(C2(Cc3nncn3C)CCC2)cc(N2Cc3c(cc(CNC4(C)CCC4)cc3C(F)(F)F)C2=O)n1] | 2-(4-(1-((4-methyl-4H-1,2,4-triazol-3- yl)methyl)cyclobutyl)-6-propylpyridin-2- yl)-6-(((1- methylcyclobutyl)amino)methyl)-4- (trifluoromethyl)isoindolin-1-one |
| 344 | 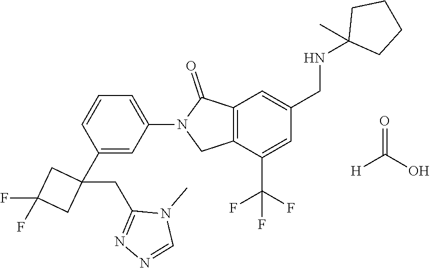 [SMILES: Cn1cnnc1CC1(c2cccc(N3Cc4c(cc(CNC5(C)CCCC5)cc4C(F)(F)F)C3=O)c2)CC(F)(F)C1 ; O=CO] | 2-(3-(3,3-difluoro-1-((4-methyl-4H- 1,2,4-triazol-3- yl)methyl)cyclobutyl)phenyl)-6-(((1- methylcyclopentyl)amino)methyl)-4- (trifluoromethyl)isoindolin-1-one formate |
| 345 | 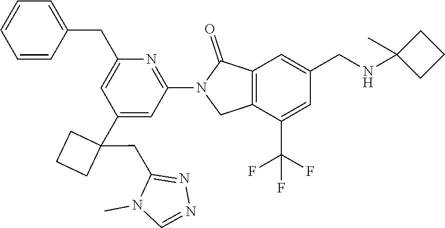 [SMILES: Cn1cnnc1CC1(c2cc(Cc3ccccc3)nc(N3Cc4c(cc(CNC5(C)CCC5)cc4C(F)(F)F)C3=O)c2)CCC1] | 2-(6-benzyl-4-(1-((4-methyl-4H-1,2,4- triazol-3-yl)methyl)cyclobutyl)pyridin-2- yl)-6-(((1- methylcyclobutyl)amino)methyl)-4- (trifluoromethyl)isoindolin-1-one |
| 346 | 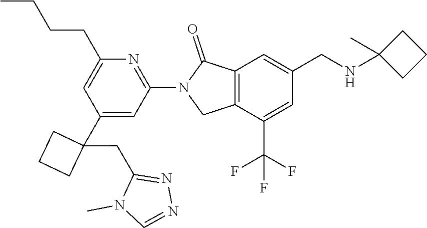 [SMILES: CCCCc1cc(C2(Cc3nncn3C)CCC2)cc(N2Cc3c(cc(CNC4(C)CCC4)cc3C(F)(F)F)C2=O)n1] | 2-(6-butyl-4-(1-((4-methyl-4H-1,2,4- triazol-3-yl)methyl)cyclobutyl)pyridin-2- yl)-6-(((1- methylcyclobutyl)amino)methyl)-4- (trifluoromethyl)isoindolin-1-one |
| 347 | 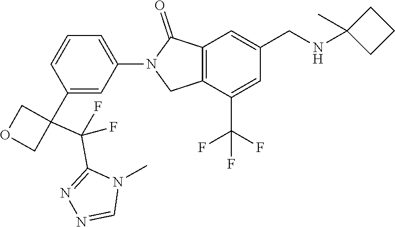 [SMILES: Cn1cnnc1C(F)(F)C1(c2cccc(N3Cc4c(cc(CNC5(C)CCC5)cc4C(F)(F)F)C3=O)c2)COC1] | 2-(3-(3-(difluoro(4-methyl-4H-1,2,4- triazol-3-yl)methyl)oxetan-3-yl)phenyl)- 6-(((1-methylcyclobutyl)amino)methyl)- 4-(trifluoromethyl)isoindolin-1-one |
| 348 | 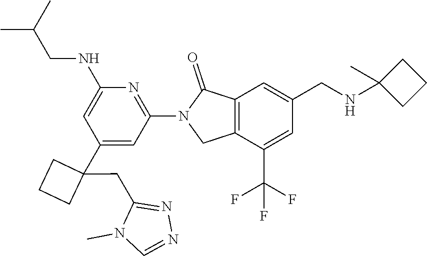 [SMILES: CC(C)CNc1cc(C2(Cc3nncn3C)CCC2)cc(N2Cc3c(cc(CNC4(C)CCC4)cc3C(F)(F)F)C2=O)n1] | 2-(6-(isobutylamino)-4-(1-((4-methyl- 4H-1,2,4-triazol-3- yl)methyl)cyclobutyl)pyridin-2-yl)-6-(((1- methylcyclobutyl)amino)methyl)-4- (trifluoromethyl)isoindolin-1-one |
| 349 | 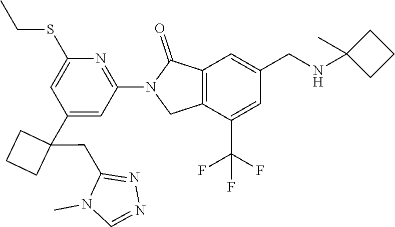 [SMILES: CCSc1cc(C2(Cc3nncn3C)CCC2)cc(N2Cc3c(cc(CNC4(C)CCC4)cc3C(F)(F)F)C2=O)n1] | 2-(6-(ethylthio)-4-(1-((4-methyl-4H- 1,2,4-triazol-3- yl)methyl)cyclobutyl)pyridin-2-yl)-6-(((1- methylcyclobutyl)amino)methyl)-4- (trifluoromethyl)isoindolin-1-one |
| 350 | 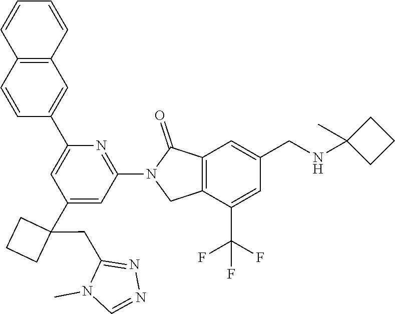 [SMILES: Cn1cnnc1CC1(c2cc(-c3ccc4ccccc4c3)nc(N3Cc4c(cc(CNC5(C)CCC5)cc4C(F)(F)F)C3=O)c2)CCC1] | 2-(4-(1-((4-methyl-4H-1,2,4-triazol-3- yl)methyl)cyclobutyl)-6-(naphthalen-2- yl)pyridin-2-yl)-6-(((1- methylcyclobutyl)amino)methyl)-4- (trifluoromethyl)isoindolin-1-one |
| 351 | 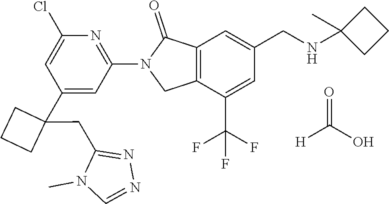 | 2-(6-chloro-4-(1-((4-methyl-4H-1,2,4- triazol-3-yl)methyl)cyclobutyl)pyridin-2- yl)-6-(((1- methylcyclobutyl)amino)methyl)-4- (trifluoromethyl)isoindolin-1-one formate |
| 352 | 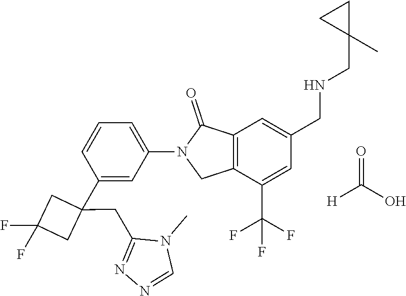 [SMILES: Cn1cnnc1CC1(c2cccc(N3Cc4c(cc(CNCC5(C)CC5)cc4C(F)(F)F)C3=O)c2)CC(F)(F)C1 ; O=CO] | 2-(3-(3,3-difluoro-1-((4-methy-4H- 1,2,4-triazol-3- yl)methyl)cyclobutyl)phenyl)-6-((((1- methylcyclopropyl)methyl)amino) methyl)-4-(trifluoromethyl)isoindolin- 1-one formate |
| 353 | 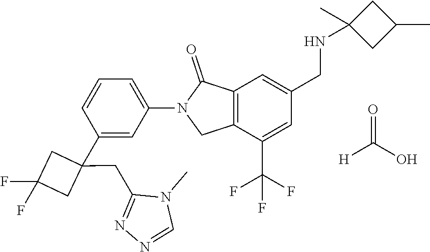 [SMILES: CC1CC(C)(NCc2cc3c(c(C(F)(F)F)c2)CN(c2cccc(C4(Cc5nncn5C)CC(F)(F)C4)c2)C3=O)C1 ; O=CO] | 2-(3-(3,3-difluoro-1-((4-methyl-4H- 1,2,4-triazol-3- yl)methyl)cyclobutyl)phenyl)-6-(((1,3- dimethylcyclobutyl)amino)methyl)-4- (trifluoromethyl)isoindolin-1-one formate |
| 354 | 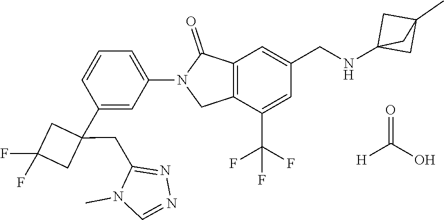 [SMILES: Cn1cnnc1CC1(c2cccc(N3Cc4c(cc(CNC56CC(C)(C5)C6)cc4C(F)(F)F)C3=O)c2)CC(F)(F)C1 ; O=CO] | 2-(3-(3,3-difluoro-1-((4-methyl-4H- 1,2,4-triazol-3- yl)methyl)cyclobutyl)phenyl)-6-(((3- methylbicyclo[1.1.1]pentan-1- yl)amino)methyl)-4- (trifluoromethyl)isoindolin-1-one formate |
| 355 | 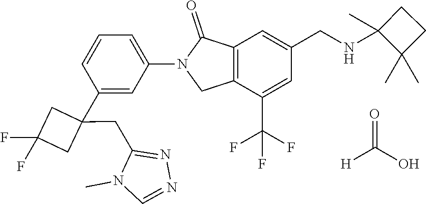 | 2-(3-(3,3-difluoro-1-((4-methyl-4H- 1,2,4-triazol-3- yl)methyl)cyclobutyl)phenyl)-4- (trifluoromethyl)-6-(((1,2,2- trimethylcyclobutyl)amino)methyl) isoindin-1-one formate |
| 356 | 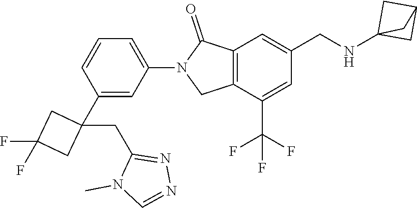 [SMILES: Cn1cnnc1CC1(c2cccc(N3Cc4c(cc(CNC56CC(C5)C6)cc4C(F)(F)F)C3=O)c2)CC(F)(F)C1] | 6-((bicyclo[1.1.1]pentan-1- ylamino)methyl)-2-(3-(3,3-difluoro-1- ((4-methyl-4H-1,2,4-triazol-3- yl)methyl)cyclobutyl)phenyl)-4- (trifluoromethyl)isoindolin-1-one |
| 357 | 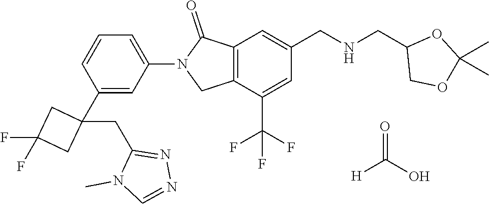 [SMILES: Cn1cnnc1CC1(c2cccc(N3Cc4c(cc(CNCC5COC(C)(C)O5)cc4C(F)(F)F)C3=O)c2)CC(F)(F)C1 ; O=CO] | 2-(3-(3,3-difluoro-1-((4-methyl-4H- 1,2,4-triazol-3- yl)methyl)cyclobutyl)phenyl)-6-((((2,2- dimethyl-1,3-dioxolan-4- yl)methyl)amino)methyl)-4- (trifluoromethyl)isoindolin-1-one formate |
| 358 | 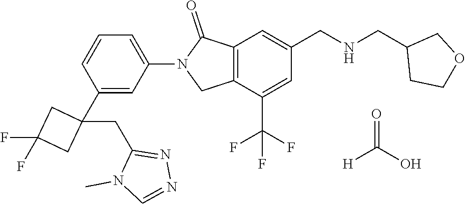 [SMILES: Cn1cnnc1CC1(c2cccc(N3Cc4c(cc(CNCC5CCOC5)cc4C(F)(F)F)C3=O)c2)CC(F)(F)C1 ; O=CO] | 2-(3-(3,3-difluoro-1-((4-methyl-4H- 1,2,4-triazol-3- yl)methyl)cyclobutyl)phenyl)-6- ((((tetrahydrofuran-3- yl)methyl)amino)methyl)-4- (trifluoromethyl)isoindolin-1-one formate |
| 359 | 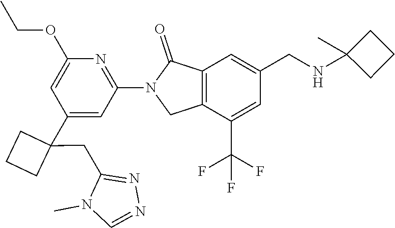 [SMILES: CCOc1cc(C2(Cc3nncn3C)CCC2)cc(N2Cc3c(cc(CNC4(C)CCC4)cc3C(F)(F)F)C2=O)n1] | 2-(6-ethoxy-4-(1-((4-methyl-4H-1,2,4- triazol-3-yl)methyl)cyclobutyl)pyridin-2- yl)-6-(((1- methylcyclobutyl)amino)methyl)-4- (trifluoromethyl)isoindolin-1-one |
| 360 | 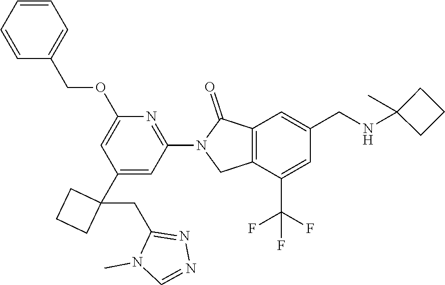 [SMILES: Cn1cnnc1CC1(c2cc(OCc3ccccc3)nc(N3Cc4c(cc(CNC5(C)CCC5)cc4C(F)(F)F)C3=O)c2)CCC1] | 2-(6-(benzyloxy)-4-(1-((4-methyl-4H- 1,2,4-triazol-3- yl)methyl)cyclobutyl)pyridin-2-yl)-6-(((1- methylcyclobutyl)amino)methyl)-4- (trifluoromethyl)isoindolin-1-one |
| 361 | 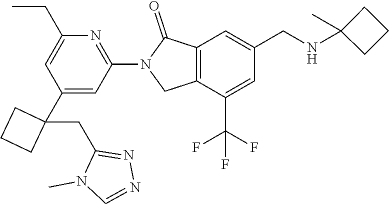 [SMILES: CCc1cc(C2(Cc3nncn3C)CCC2)cc(N2Cc3c(cc(CNC4(C)CCC4)cc3C(F)(F)F)C2=O)n1] | 2-(6-ethyl-4-(1-((4-methyl-4H-1,2,4- triazol-3-yl)methyl)cyclobutyl)pyridin-2- yl)-6-(((1- methylcyclobutyl)amino)methyl)-4- (trifluoromethyl)isoindolin-1-one |
| 362 | 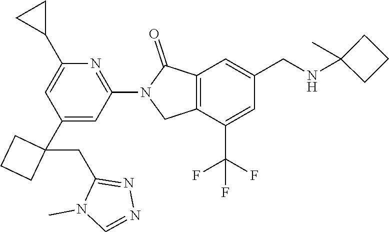 [SMILES: Cn1cnnc1CC1(c2cc(C3CC3)nc(N3Cc4c(cc(CNC5(C)CCC5)cc4C(F)(F)F)C3=O)c2)CCC1] | 2-(6-cyclopropyl-4-(1-((4-methyl-4H- 1,2,4-triazol-3- yl)methyl)cyclobutyl)pyridin-2-yl)-6-(((1- methylcyclobutyl)amino)methyl)-4- (trifluoromethyl)isoindolin-1-one |
| 363 | 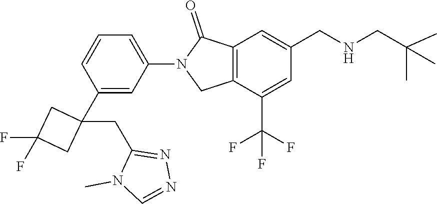 [SMILES: Cn1cnnc1CC1(c2cccc(N3Cc4c(cc(CNCC(C)(C)C)cc4C(F)(F)F)C3=O)c2)CC(F)(F)C1] | 2-(3-(3,3-difluoro-1-((4-methyl-4H- 1,2,4-triazol-3- yl)methyl)cyclobutyl)phenyl)-6- ((neopentylamino)methyl)-4- (trifluoromethyl)isoindolin-1-one |
| 364 | 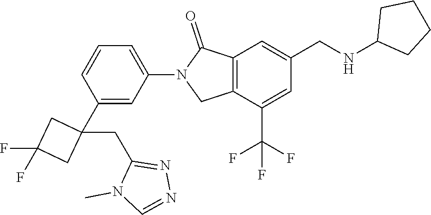 [SMILES: Cn1cnnc1CC1(c2cccc(N3Cc4c(cc(CNC5CCCC5)cc4C(F)(F)F)C3=O)c2)CC(F)(F)C1] | 6-((cyclopentylamino)methyl)-2-(3-(3,3- difluoro-1-((4-methyl-4H-1,2,4-triazol-3- yl)methyl)cyclobutyl)phenyl)-4- (trifluoromethyl)isoindolin-1-one |
| 365 | 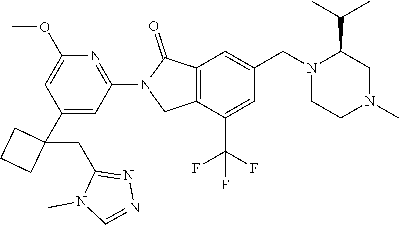 [SMILES: COc1cc(C2(Cc3nncn3C)CCC2)cc(N2Cc3c(cc(CN4CCN(C)C[C@@H]4C(C)C)cc3C(F)(F)F)C2=O)n1] | (S)-6-((2-isopropyl-4-methylpiperazin- 1-yl)methyl)-2-(6-methoxy-4-(1-((4- methyl-4H-1,2,4-triazol-3- yl)methyl)cyclobutyl)pyridin-2-yl)-4- (trifluoromethyl)isoindolin-1-one |
| 366 | 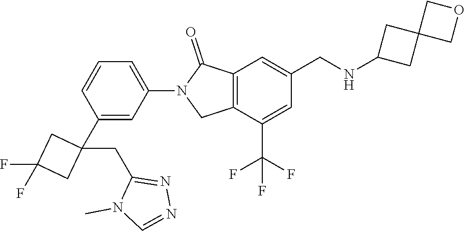 [SMILES: Cn1cnnc1CC1(c2cccc(N3Cc4c(cc(CNC5CC6(COC6)C5)cc4C(F)(F)F)C3=O)c2)CC(F)(F)C1] | 6-((2-oxaspiro[3.3]heptan-6- ylamino)methyl)-2-(3-(3,3-difluoro-1- ((4-methyl-4H-1,2,4-triazol-3- yl)methyl)cyclobutyl)phenyl)-4- (trifluoromethyl)isoindolin-1-one |
| 367 | 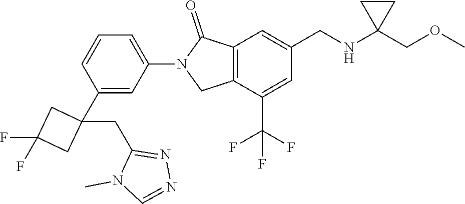 [SMILES: COCC1(NCc2cc3c(c(C(F)(F)F)c2)CN(c2cccc(C4(Cc5nncn5C)CC(F)(F)C4)c2)C3=O)CC1] | 2-(3-(3,3-difluoro-1-((4-methyl-4H- 1,2,4-triazol-3- yl)methyl)cyclobutyl)phenyl)-6-(((1- (methoxymethyl)cyclopropyl)amino) methyl)-4-(trifluoromethyl)isoindolin- 1-one |
| 368 | 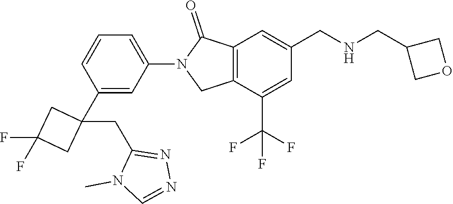 [SMILES: Cn1cnnc1CC1(c2cccc(N3Cc4c(cc(CNCC5COC5)cc4C(F)(F)F)C3=O)c2)CC(F)(F)C1] | 2-(3-(3,3-difluoro-1-((4-methyl-4H- 1,2,4-triazol-3- yl)methyl)cyclobutyl)phenyl)-6- (((oxetan-3-ylmethyl)amino)methyl)-4- (trifluoromethyl)isoindolin-1-one |
| 369 | 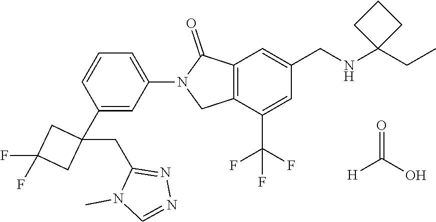 [SMILES: CCC1(NCc2cc3c(c(C(F)(F)F)c2)CN(c2cccc(C4(Cc5nncn5C)CC(F)(F)C4)c2)C3=O)CCC1 ; O=CO] | 2-(3-(3,3-difluoro-1-((4-methyl-4H- 1,2,4-triazol-3- yl)methyl)cyclobutyl)phenyl)-6-(((1- ethylcyclobutyl)amino)methyl)-4- (trifluoromethyl)isoindolin-1-one formate |
| 370 | 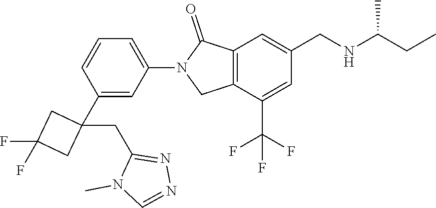 [SMILES: CC[C@@H](C)NCc1cc2c(c(C(F)(F)F)c1)CN(c1cccc(C3(Cc4nncn4C)CC(F)(F)C3)c1)C2=O] | (R)-6-((sec-butylamino)methyl)-2-(3- (3,3-difluoro-1-((4-methyl-4H-1,2,4- triazol-3-yl)methyl)cyclobutyl)phenyl)-4- (trifluoromethyl)isoindolin-1-one |
| 371 | 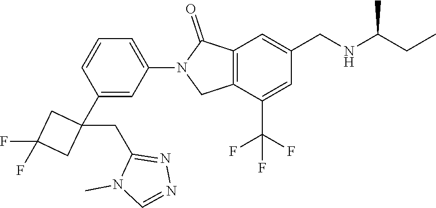 [SMILES: CC[C@H](C)NCc1cc2c(c(C(F)(F)F)c1)CN(c1cccc(C3(Cc4nncn4C)CC(F)(F)C3)c1)C2=O] | (S)-6-((sec-butylamino)methyl)-2-(3- (3,3-difluoro-1-((4-methyl-4H-1,2,4- triazol-3-yl)methyl)cyclobutyl)phenyl)-4- (trifluoromethyl)isoindolin-1-one |
| 372 | 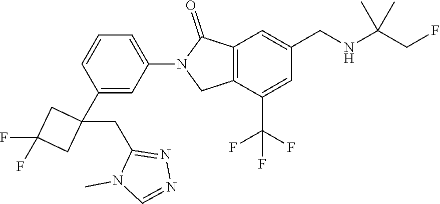 [SMILES: Cn1cnnc1CC1(c2cccc(N3Cc4c(cc(CNC(C)(C)CF)cc4C(F)(F)F)C3=O)c2)CC(F)(F)C1] | 2-(3-(3,3-difluoro-1-((4-methyl-4H- 1,2,4-triazol-3- yl)methyl)cyclobutyl)phenyl)-6-(((1- fluoro-2-methylpropan-2- yl)amino)methyl)-4- (trifluoromethyl)isoindolin-1-one |
| 373 | 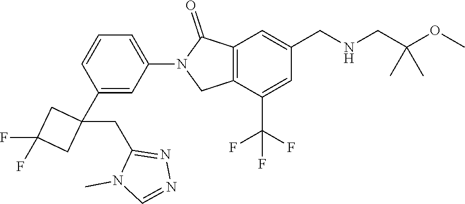 [SMILES: COC(C)(C)CNCc1cc2c(c(C(F)(F)F)c1)CN(c1cccc(C3(Cc4nncn4C)CC(F)(F)C3)c1)C2=O] | 2-(3-(3,3-difluoro-1-((4-methy-4H- 1,2,4-triazol-3- yl)methyl)cyclobutyl)phenyl)-6-(((2- methoxy-2- methylpropyl)amino)methyl)-4- (trifluoromethyl)isoindolin-1-one |
| 374 | 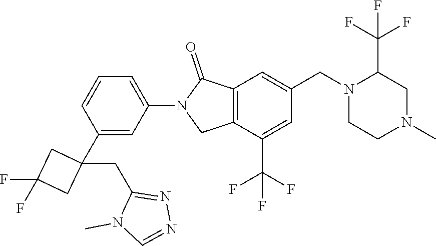 [SMILES: CN1CCN(Cc2cc3c(c(C(F)(F)F)c2)CN(c2cccc(C4(Cc5nncn5C)CC(F)(F)C4)c2)C3=O)C(C(F)(F)F)C1] | 2-(3-(3,3-difluoro-1-((4-methyl-4H- 1,2,4-triazol-3- yl)methyl)cyclobutyl)phenyl)-6-((4- methyl-2-(trifluoromethyl)piperazin-1- yl)methyl)-4-(trifluoromethyl)isoindolin- 1-one |
| 375 | 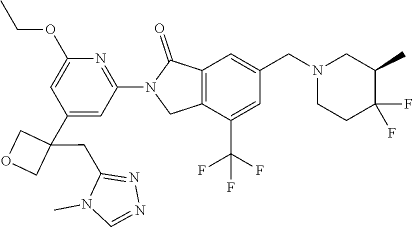 [SMILES: CCOc1cc(C2(Cc3nncn3C)COC2)cc(N2Cc3c(cc(CN4CCC(F)(F)[C@H](C)C4)cc3C(F)(F)F)C2=O)n1] | (R)-6-((4,4-difluoro-3-methylpiperidin-1- yl)methyl)-2-(6-ethoxy-4-(3-((4-methyl- 4H-1,2,4-triazol-3-yl)methyl)oxetan-3- yl)pyridin-2-yl)-4- (trifluoromethyl)isoindolin-1-one |
| 376 | 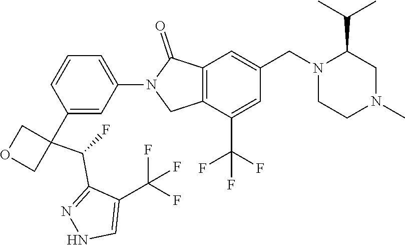 [SMILES: CC(C)[C@H]1CN(C)CCN1Cc1cc2c(c(C(F)(F)F)c1)CN(c1cccc(C3([C@H](F)c4n[nH]cc4C(F)(F)F)COC3)c1)C2=O] | 2-(3-(3-((S)-fluoro(4-(trifluoromethyl)- 1H-pyrazol-3-yl)methyl)oxetan-3- yl)phenyl)-6-(((S)-2-isopropyl-4- methylpiperazin-1-yl)methyl)-4- (trifluoromethyl)isoindolin-1-one |
| 377 | 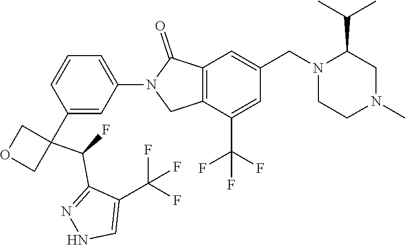 [SMILES: CC(C)[C@H]1CN(C)CCN1Cc1cc2c(c(C(F)(F)F)c1)CN(c1cccc(C3([C@@H](F)c4n[nH]cc4C(F)(F)F)COC3)c1)C2=O] | 2-(3-(3-((R)-fluoro(4-(trifluoromethyl)- 1H-pyrazol-3-yl)methyl)oxetan-3- yl)phenyl)-6-(((S)-2-isopropyl-4- methylpiperazin-1-yl)methyl)-4- (trifluoromethyl)isoindolin-1-one |
| 378 | 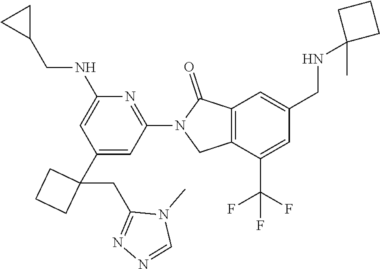 [SMILES: Cn1cnnc1CC1(c2cc(NCC3CC3)nc(N3Cc4c(cc(CNC5(C)CCC5)cc4C(F)(F)F)C3=O)c2)CCC1] | 2-(6-((cyclopropylmethyl)amino)-4-(1- ((4-methyl-4H-1,2,4-triazol-3- yl)methyl)cyclobutyl)pyridin-2-yl)-6-(((1- methylcyclobutyl)amino)methyl)-4- (trifluoromethyl)isoindolin-1-one |
| 379 | 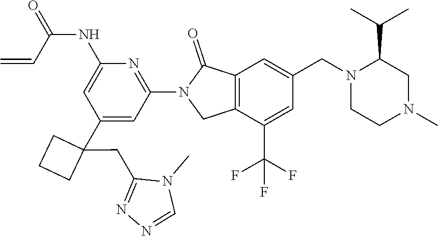 [SMILES: C=CC(=O)Nc1cc(C2(Cc3nncn3C)CCC2)cc(N2Cc3c(cc(CN4CCN(C)C[C@@H]4C(C)C)cc3C(F)(F)F)C2=O)n1] | (S)-N-(6-(6-((2-isopropyl-4- methylpiperazin-1-yl)methyl)-1-oxo-4- (trifluoromethyl)isoindolin-2-yl)-4-(1-((4- methyl-4H-1,2,4-triazol-3- yl)methyl)cyclobutyl)pyridin-2- yl)acrylamide |
| 380 | 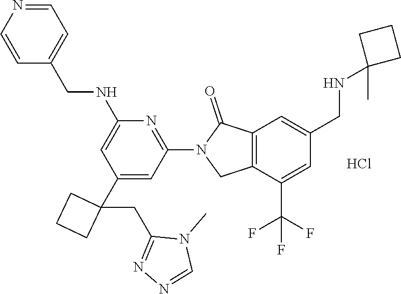 [SMILES: Cn1cnnc1CC1(c2cc(NCc3ccncc3)nc(N3Cc4c(cc(CNC5(C)CCC5)cc4C(F)(F)F)C3=O)c2)CCC1 ; Cl] | 2-(4-(1-((4-methyl-4H-1,2,4-triazol-3- yl)methyl)cyclobutyl)-6-((pyridin-4- ylmethyl)amino)pyridin-2-yl)-6-(((1- methylcyclobutyl)amino)methyl)-4- (trifluoromethyl)isoindolin-1-one hydrochloride |
| 381 | 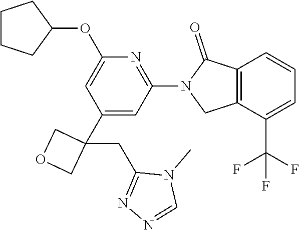 [SMILES: Cn1cnnc1CC1(c2cc(OC3CCCC3)nc(N3Cc4c(cccc4C(F)(F)F)C3=O)c2)COC1] | 2-(6-(cyclopentyloxy)-4-(3-((4-methyl- 4H-1,2,4-triazol-3-yl)methyl)oxetan-3- yl)pyridin-2-yl)-4- (trifluoromethyl)isoindolin-1-one |
| 382 | 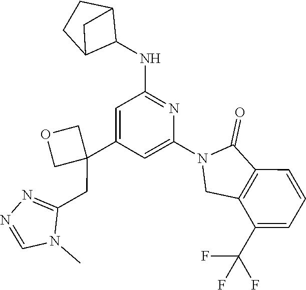 [SMILES: Cn1cnnc1CC1(c2cc(NC3C4CCC3C4)nc(N3Cc4c(cccc4C(F)(F)F)C3=O)c2)COC1] | 2-(6-(bicyclo[2.1.1]hexan-5-ylamino)-4- (3-((4-methyl-4H-1,2,4-triazol-3- yl)methyl)oxetan-3-yl)pyridin-2-yl)-4- (trifluoromethyl)isoindolin-1-one |
| 383 | 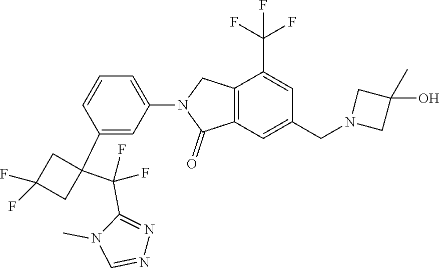 [SMILES: Cn1cnnc1C(F)(F)C1(c2cccc(N3Cc4c(cc(CN5CC(C)(O)C5)cc4C(F)(F)F)C3=O)c2)CC(F)(F)C1] | 2-(3-(1-(difluoro(4-methyl-4H-1,2,4- triazol-3-yl)methyl)-3,3- difluorocyclobutyl)phenyl)-6-((3- hydroxy-3-methylazetidin-1-yl)methyl)- 4-(trifluoromethyl)isoindolin-1-one |
| 384 | 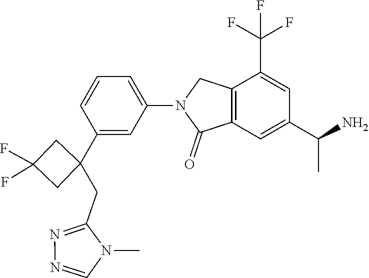 [SMILES: C[C@H](N)c1cc2c(c(C(F)(F)F)c1)CN(c1cccc(C3(Cc4nncn4C)CC(F)(F)C3)c1)C2=O] | (S)-6-(1-aminoethyl)-2-(3-(3,3-difluoro- 1-((4-methyl-4H-1,2,4-triazol-3- yl)methyl)cyclobutyl)phenyl)-4- (trifluoromethyl)isoindolin-1-one |
| 385 | 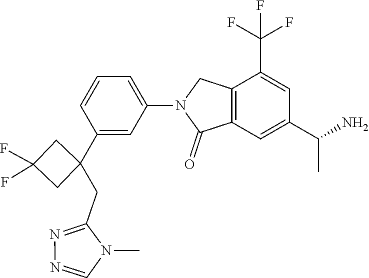 [SMILES: C[C@@H](N)c1cc2c(c(C(F)(F)F)c1)CN(c1cccc(C3(Cc4nncn4C)CC(F)(F)C3)c1)C2=O] | (R)-6-(1-aminoethyl)-2-(3-(3,3-difluoro- 1-((4-methyl-4H-1,2,4-triazol-3- yl)methyl)cyclobutyl)phenyl)-4- (trifluoromethyl)isoindolin-1-one |
| 386 | 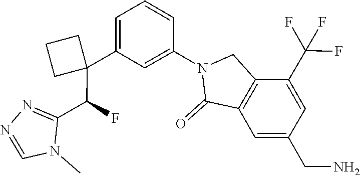 [SMILES: Cn1cnnc1[C@H](F)C1(c2cccc(N3Cc4c(cc(CN)cc4C(F)(F)F)C3=O)c2)CCC1] | (R)-6-(aminomethyl)-2-(3-(1-(fluoro(4- methyl-4H-1,2,4-triazol-3- yl)methyl)cyclobutyl)phenyl)-4- (trifluoromethyl)isoindolin-1-one |
| 387 | 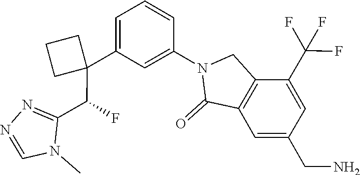 [SMILES: Cn1cnnc1[C@@H](F)C1(c2cccc(N3Cc4c(cc(CN)cc4C(F)(F)F)C3=O)c2)CCC1] | (S)-6-(aminomethyl)-2-(3-(1-(fluoro(4- methyl-4H-1,2,4-triazol-3- yl)methyl)cyclobutyl)phenyl)-4- (trifluoromethyl)isoindolin-1-one |
| 388 | 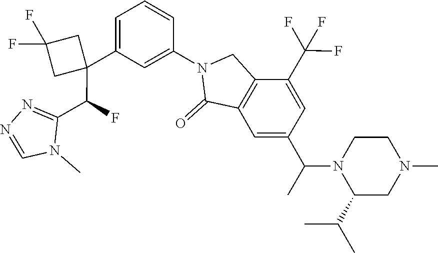 [SMILES: CC(C)[C@H]1CN(C)CCN1C(C)c1cc2c(c(C(F)(F)F)c1)CN(c1cccc(C3([C@@H](F)c4nncn4C)CC(F)(F)C3)c1)C2=O] | 2-(3-(3,3-difluoro-1-((R)-fluoro(4- methyl-4H-1,2,4-triazol-3- yl)methyl)cyclobutyl)phenyl)-6-(1-((S)- 2-isopropyl-4-methylpiperazin-1- yl)ethyl)-4-(trifluoromethyl)isoindolin-1- one |
| 389 | 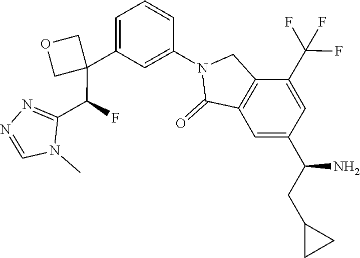 [SMILES: Cn1cnnc1[C@H](F)C1(c2cccc(N3Cc4c(cc([C@@H](N)CC5CC5)cc4C(F)(F)F)C3=O)c2)COC1] | 6-((S)-1-amino-2-cyclopropylethyl)-2- (3-(3-((R)-fluoro(4-methyl-4H-1,2,4- triazol-3-yl)methyl)oxetan-3-yl)phenyl)- 4-(trifluoromethyl)isoindolin-1-one |
| 390 | 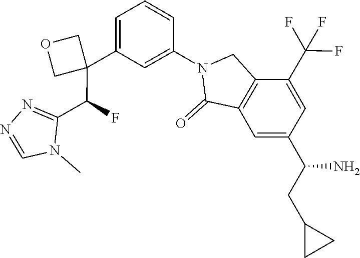 [SMILES: Cn1cnnc1[C@H](F)C1(c2cccc(N3Cc4c(cc([C@H](N)CC5CC5)cc4C(F)(F)F)C3=O)c2)COC1] | 6-((R)-1-amino-2-cyclopropylethyl)-2- (3-(3-((R)-fluoro(4-methyl-4H-1,2,4- triazol-3-yl)methyl)oxetan-3-yl)phenyl)- 4-(trifluoromethyl)isoindolin-1-one |
| 391 | 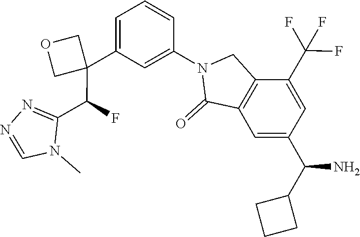 [SMILES: Cn1cnnc1[C@H](F)C1(c2cccc(N3Cc4c(cc([C@@H](N)C5CCC5)cc4C(F)(F)F)C3=O)c2)COC1] | 6-((S)-amino(cyclobutyl)methyl)-2-(3- (3-((R)-fluoro(4-methyl-4H-1,2,4-triazol- 3-yl)methyl)oxetan-3-yl)phenyl)-4- (trifluoromethyl)isoindolin-1-one |
| 392 | 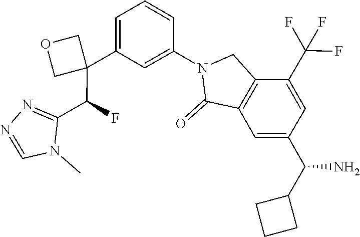 [SMILES: Cn1cnnc1[C@H](F)C1(c2cccc(N3Cc4c(cc([C@H](N)C5CCC5)cc4C(F)(F)F)C3=O)c2)COC1] | 6-((R)-amino(cyclobutyl)methyl)-2-(3- (3-((R)-fluoro(4-methyl-4H-1,2,4-triazol- 3-yl)methyl)oxetan-3-yl)phenyl)-4- (trifluoromethyl)isoindolin-1-one |
| 393 | 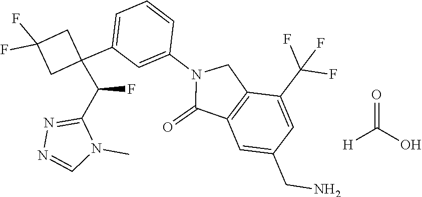 [SMILES: Cn1cnnc1[C@H](F)C1(c2cccc(N3Cc4c(cc(CN)cc4C(F)(F)F)C3=O)c2)CC(F)(F)C1 ; O=CO] | (R)-6-(aminomethyl)-2-(3-(3,3-difluoro- 1-(fluoro(4-methyl-4H-1,2,4-triazol-3- yl)methyl)cyclobutyl)phenyl)-4- (trifluoromethyl)isoindolin-1-one formate |
| 394 | 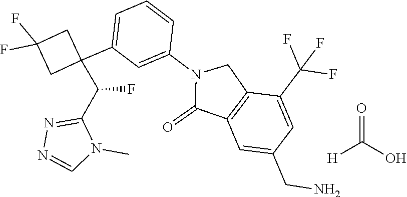 | (S)-6-(aminomethyl)-2-(3-(3,3-difluoro- 1-(fluoro(4-methyl-4H-1,2,4-triazol-3- yl)methyl)cyclobutyl)phenyl)-4- (trifluoromethyl)isoindolin-1-one formate |
| 395 | 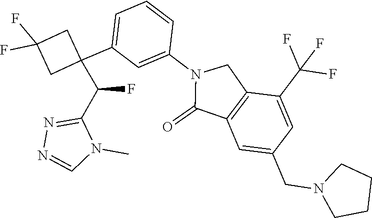 [SMILES: Cn1cnnc1[C@H](F)C1(c2cccc(N3Cc4c(cc(CN5CCCC5)cc4C(F)(F)F)C3=O)c2)CC(F)(F)C1] | (R)-2-(3-(3,3-difluoro-1-(fluoro(4- methyl-4H-1,2,4-triazol-3- yl)methyl)cyclobutyl)phenyl)-6- (pyrrolidin-1-ylmethyl)-4- (trifluoromethyl)isoindolin-1-one |
| 396 | 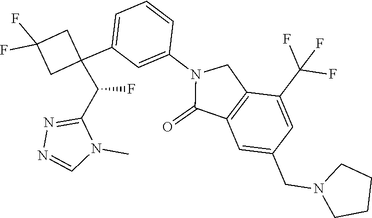 [SMILES: Cn1cnnc1[C@@H](F)C1(c2cccc(N3Cc4c(cc(CN5CCCC5)cc4C(F)(F)F)C3=O)c2)CC(F)(F)C1] | (S)-2-(3-(3,3-difluoro-1-(fluoro(4- methyl-4H-1,2,4-triazol-3- yl)methyl)cyclobutyl)phenyl)-6- (pyrrolidin-1-ylmethyl)-4- (trifluoromethyl)isoindolin-1-one |
| 397 | 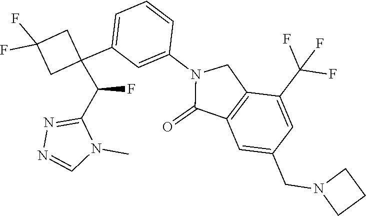 [SMILES: Cn1cnnc1[C@H](F)C1(c2cccc(N3Cc4c(cc(CN5CCC5)cc4C(F)(F)F)C3=O)c2)CC(F)(F)C1] | (R)-6-(azetidin-1-ylmethyl)-2-(3-(3,3- difluoro-1-(fluoro(4-methyl-4H-1,2,4- triazol-3-yl)methyl)cyclobutyl)phenyl)-4- (trifluoromethyl)isoindolin-1-one |
| 398 | 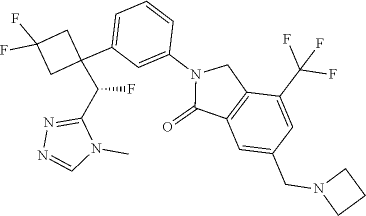 [SMILES: Cn1cnnc1[C@@H](F)C1(c2cccc(N3Cc4c(cc(CN5CCC5)cc4C(F)(F)F)C3=O)c2)CC(F)(F)C1] | (S)-6-(azetidin-1-ylmethyl)-2-(3-(3,3- difluoro-1-(fluoro(4-methyl-4H-1,2,4- triazol-3-yl)methyl)cyclobutyl)phenyl)-4- (trifluoromethyl)isoindolin-1-one |
| 399 | 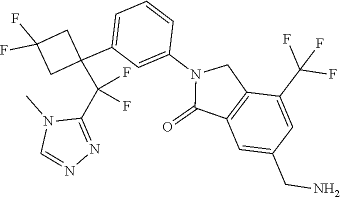 [SMILES: Cn1cnnc1C(F)(F)C1(c2cccc(N3Cc4c(cc(CN)cc4C(F)(F)F)C3=O)c2)CC(F)(F)C1] | 6-(aminomethyl)-2-(3-(1-(difluoro(4- methyl-4H-1,2,4-triazol-3-yl)methyl)- 3,3-difluorocyclobutyl)phenyl)-4- (trifluoromethyl)isoindolin-1-one |
| 400 | 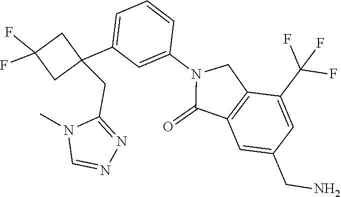 [SMILES: Cn1cnnc1CC1(c2cccc(N3Cc4c(cc(CN)cc4C(F)(F)F)C3=O)c2)CC(F)(F)C1] | 6-(aminomethyl)-2-(3-(3,3-difluoro-1- ((4-methyl-4H-1,2,4-triazol-3- yl)methyl)cyclobutyl)phenyl)-4- (trifluoromethyl)isoindolin-1-one |
| 401 | 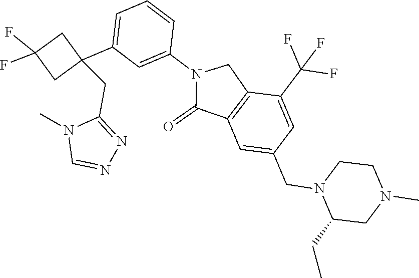 [SMILES: CC[C@H]1CN(C)CCN1Cc1cc2c(c(C(F)(F)F)c1)CN(c1cccc(C3(Cc4nncn4C)CC(F)(F)C3)c1)C2=O] | (S)-2-(3-(3,3-difluoro-1-((4-methyl-4H- 1,2,4-triazol-3- yl)methyl)cyclobutyl)phenyl)-6-((2- ethyl-4-methylpiperazin-1-yl)methyl)-4- (trifluoromethyl)isoindolin-1-one |
| 402 | 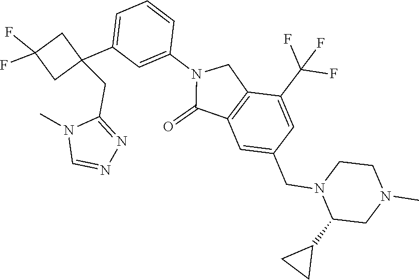 [SMILES: CN1CCN(Cc2cc3c(c(C(F)(F)F)c2)CN(c2cccc(C4(Cc5nncn5C)CC(F)(F)C4)c2)C3=O)[C@@H](C2CC2)C1] | (S)-6-((2-cyclopropyl-4- methylpiperazin-1-yl)methyl)-2-(3-(3,3- difluoro-1-((4-methyl-4H-1,2,4-triazol-3- yl)methyl)cyclobutyl)phenyl)-4- (trifluoromethyl)isoindolin-1-one |
| 403 | 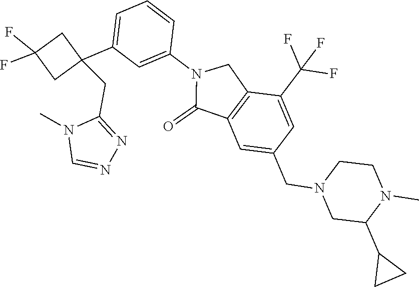 [SMILES: CN1CCN(Cc2cc3c(c(C(F)(F)F)c2)CN(c2cccc(C4(Cc5nncn5C)CC(F)(F)C4)c2)C3=O)CC1C1CC1] | 6-((3-cyclopropyl-4-methylpiperazin-1- yl)methyl)-2-(3-(3,3-difluoro-1-((4- methyl-4H-1,2,4-triazol-3- yl)methyl)cyclobutyl)phenyl)-4- (trifluoromethyl)isoindolin-1-one |
| 404 | 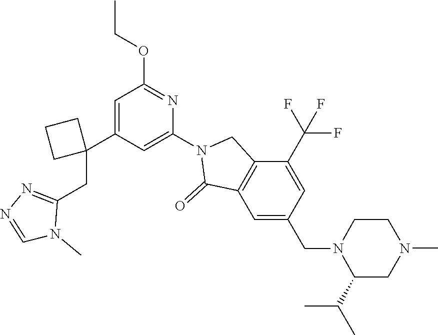 [SMILES: CCOc1cc(C2(Cc3nncn3C)CCC2)cc(N2Cc3c(cc(CN4CCN(C)C[C@@H]4C(C)C)cc3C(F)(F)F)C2=O)n1] | (S)-2-(6-ethoxy-4-(1-((4-methyl-4H- 1,2,4-triazol-3- yl)methyl)cyclobutyl)pyridin-2-yl)-6-((2- isopropyl-4-methylpiperazin-1 - yl)methyl)-4-(trifluoromethyl)isoindolin- 1-one |
| 405 | 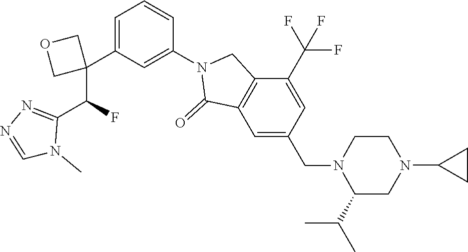 [SMILES: CC(C)[C@H]1CN(C2CC2)CCN1Cc1cc2c(c(C(F)(F)F)c1)CN(c1cccc(C3([C@@H](F)c4nncn4C)COC3)c1)C2=O] | 6-(((S)-4-cyclopropyl-2- isopropylpiperazin-1-yl)methyl)-2-(3-(3- ((R)-fluoro(4-methyl-4H-1,2,4-triazol-3- yl)methyl)oxetan-3-yl)phenyl)-4- (trifluoromethyl)isoindolin-1-one |
| 406 | 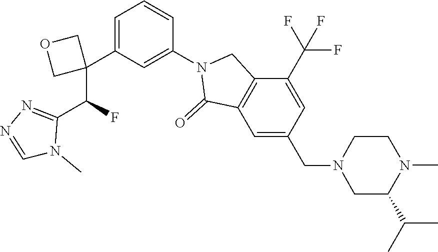 [SMILES: CC(C)[C@@H]1CN(Cc2cc3c(c(C(F)(F)F)c2)CN(c2cccc(C4([C@@H](F)c5nncn5C)COC4)c2)C3=O)CCN1C] | 2-(3-(3-((R)-fluoro(4-methyl-4H-1,2,4- triazol-3-yl)methyl)oxetan-3-yl)phenyl)- 6-(((R)-3-isopropyl-4-methylpiperazin- 1-yl)methyl)-4- (trifluoromethyl)isoindolin-1-one |
| 407 | 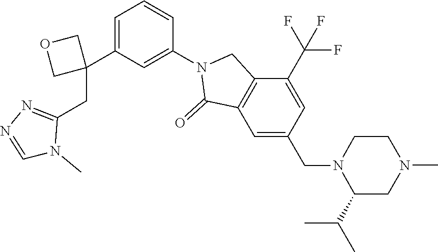 [SMILES: CC(C)[C@H]1CN(C)CCN1Cc1cc2c(c(C(F)(F)F)c1)CN(c1cccc(C3(Cc4nncn4C)COC3)c1)C2=O] | (S)-6-((2-isopropyl-4-methylpiperazin- 1-yl)methyl)-2-(3-(3-((4-methyl-4H- 1,2,4-triazol-3-yl)methyl)oxetan-3- yl)phenyl)-4-(trifluoromethyl)isoindolin- 1-one |
| 408 | 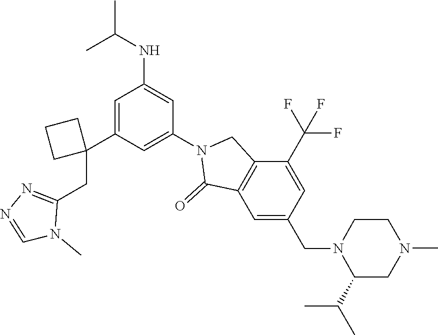 [SMILES: CC(C)Nc1cc(N2Cc3c(cc(CN4CCN(C)C[C@@H]4C(C)C)cc3C(F)(F)F)C2=O)cc(C2(Cc3nncn3C)CCC2)c1] | (S)-6-((2-isopropyl-4-methylpiperazin- 1-yl)methyl)-2-(3-(isopropylamino)-5- (1-((4-methyl-4H-1,2,4-triazol-3- yl)methyl)cyclobutyl)phenyl)-4- (trifluoromethyl)isoindolin-1-one |
| 409 | 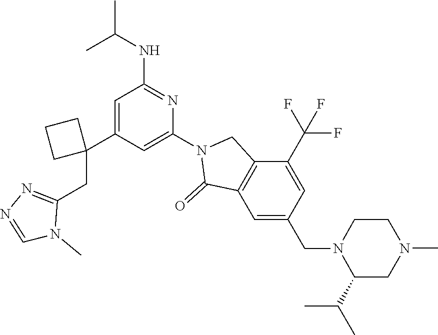 [SMILES: CC(C)Nc1cc(C2(Cc3nncn3C)CCC2)cc(N2Cc3c(cc(CN4CCN(C)C[C@@H]4C(C)C)cc3C(F)(F)F)C2=O)n1] | (S)-6-((2-isopropyl-4-methylpiperazin- 1-yl)methyl)-2-(6-(isopropylamino)-4- (1-((4-methyl-4H-1,2,4-triazol-3- yl)methyl)cyclobutyl)pyridin-2-yl)-4- (trifluoromethyl)isoindolin-1-one |
| 410 | 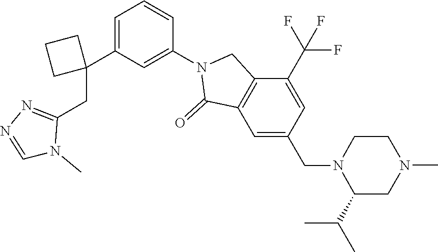 [SMILES: CC(C)[C@H]1CN(C)CCN1Cc1cc2c(c(C(F)(F)F)c1)CN(c1cccc(C3(Cc4nncn4C)CCC3)c1)C2=O] | (S)-6-((2-isopropyl-4-methylpiperazin- 1-yl)methyl)-2-(3-(1-((4-methyl-4H- 1,2,4-triazol-3- yl)methyl)cyclobutyl)phenyl)-4- (trifluoromethyl)isoindolin-1-one |
| 411 | 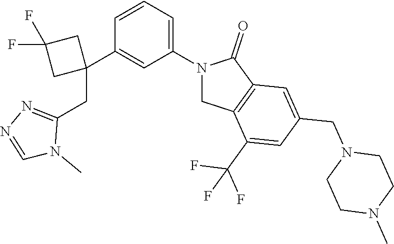 [SMILES: CN1CCN(Cc2cc3c(c(C(F)(F)F)c2)CN(c2cccc(C4(Cc5nncn5C)CC(F)(F)C4)c2)C3=O)CC1] | 2-(3-(3,3-difluoro-1-((4-methyl-4H- 1,2,4-triazol-3- yl)methyl)cyclobutyl)phenyl)-6-((4- methylpiperazin-1-yl)methyl)-4- (trifluoromethyl)isoindolin-1-one |
| 412 | 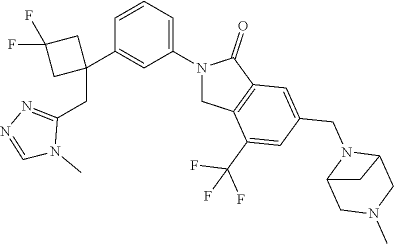 [SMILES: CN1CC2CC(C1)N2Cc1cc2c(c(C(F)(F)F)c1)CN(c1cccc(C3(Cc4nncn4C)CC(F)(F)C3)c1)C2=O] | 2-(3-(3,3-difluoro-1-((4-methyl-4H- 1,2,4-triazol-3- yl)methyl)cyclobutyl)phenyl)-6-((3- methyl-3,6-diazabicyclo[3.1.1]heptan- 6-yl)methyl)-4- (trifluoromethyl)isoindolin-1-one |
| 413 | 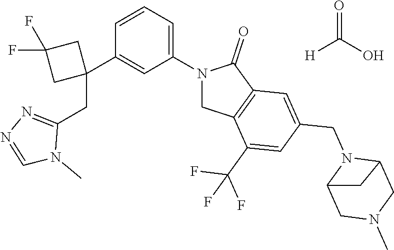 | 2-(3-(3,3-difluoro-1-((4-methyl-4H- 1,2,4-triazol-3- yl)methyl)cyclobutyl)phenyl)-6-((3- methyl-3,6-diazabicyclo[3.1.1]heptan- 6-yl)methyl)-4- (trifluoromethyl)isoindolin-1-one formate |
| 414 | 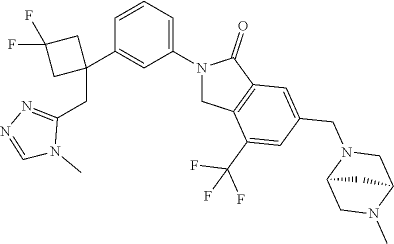 [SMILES: CN1C[C@@H]2C[C@H]1CN2Cc1cc2c(c(C(F)(F)F)c1)CN(c1cccc(C3(Cc4nncn4C)CC(F)(F)C3)c1)C2=O] | 2-(3-(3,3-difluoro-1-((4-methyl-4H- 1,2,4-triazol-3- yl)methyl)cyclobutyl)phenyl)-6- (((1S,4S)-5-methyl-2,5- diazabicyclo[2.2.1]heptan-2-yl)methyl)- 4-(trifluoromethyl)isoindolin-1-one |
| 415 | 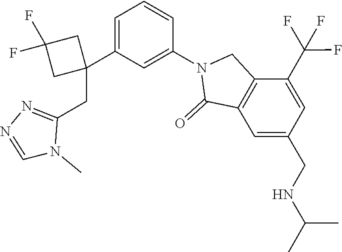 [SMILES: CC(C)NCc1cc2c(c(C(F)(F)F)c1)CN(c1cccc(C3(Cc4nncn4C)CC(F)(F)C3)c1)C2=O] | 2-(3-(3,3-difluoro-1-((4-methyl-4H- 1,2,4-triazol-3- yl)methyl)cyclobutyl)phenyl)-6- ((isopropylamino)methyl)-4- (trifluoromethyl)isoindolin-1-one |
| 416 | 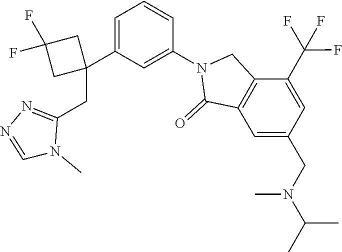 [SMILES: CC(C)N(C)Cc1cc2c(c(C(F)(F)F)c1)CN(c1cccc(C3(Cc4nncn4C)CC(F)(F)C3)c1)C2=O] | 2-(3-(3,3-difluoro-1-((4-methyl-4H- 1,2,4-triazol-3- yl)methyl)cyclobutyl)phenyl)-6- ((isopropyl(methyl)amino)methyl)-4- (trifluoromethyl)isoindolin-1-one |
| 417 | 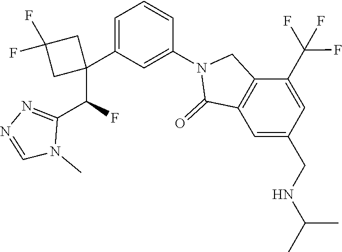 [SMILES: CC(C)NCc1cc2c(c(C(F)(F)F)c1)CN(c1cccc(C3([C@@H](F)c4nncn4C)CC(F)(F)C3)c1)C2=O] | (R)-2-(3-(3,3-difluoro-1-(fluoro(4- methyl-4H-1,2,4-triazol-3- yl)methyl)cyclobutyl)phenyl)-6- ((isopropylamino)methyl)-4- (trifluoromethyl)isoindolin-1-one |
| 418 | 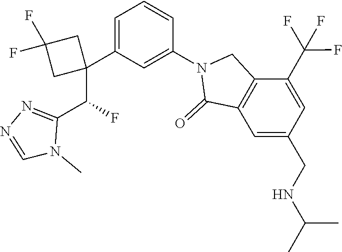 [SMILES: CC(C)NCc1cc2c(c(C(F)(F)F)c1)CN(c1cccc(C3([C@H](F)c4nncn4C)CC(F)(F)C3)c1)C2=O] | (S)-2-(3-(3,3-difluoro-1-(fluoro(4- methyl-4H-1,2,4-triazol-3- yl)methyl)cyclobutyl)phenyl)-6- ((isopropylamino)methyl)-4- (trifluoromethyl)isoindolin-1-one |
| 419 | 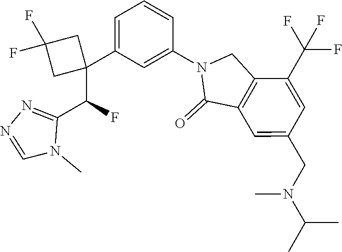 [SMILES: CC(C)N(C)Cc1cc2c(c(C(F)(F)F)c1)CN(c1cccc(C3([C@@H](F)c4nncn4C)CC(F)(F)C3)c1)C2=O] | (R)-2-(3-(3,3-difluoro-1-(fluoro(4- methyl-4H-1,2,4-triazol-3- yl)methyl)cyclobutyl)phenyl)-6- ((isopropyl(methyl)amino)methyl)-4- (trifluoromethyl)isoindolin-1-one |
| 420 | 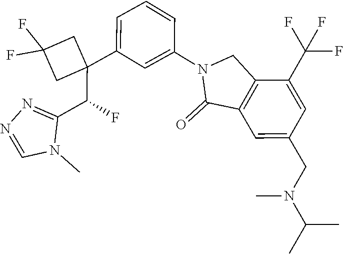 [SMILES: CC(C)N(C)Cc1cc2c(c(C(F)(F)F)c1)CN(c1cccc(C3([C@H](F)c4nncn4C)CC(F)(F)C3)c1)C2=O] | (S)-2-(3-(3,3-difluoro-1-(fluoro(4- methyl-4H-1,2,4-triazol-3- yl)methyl)cyclobutyl)phenyl)-6- ((isopropyl(methyl)amino)methyl)-4- (trifluoromethyl)isoindolin-1-one |
| 421 | 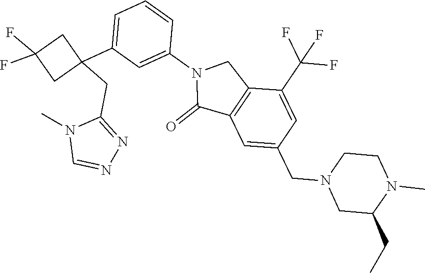 [SMILES: CC[C@H]1CN(Cc2cc3c(c(C(F)(F)F)c2)CN(c2cccc(C4(Cc5nncn5C)CC(F)(F)C4)c2)C3=O)CCN1C] | (S)-2-(3-(3,3-difluoro-1-((4-methyl-4H- 1,2,4-triazol-3- yl)methyl)cyclobutyl)phenyl)-6-((3- ethyl-4-methylpiperazin-1-yl)methyl)-4- (trifluoromethyl)isoindolin-1-one |
| 422 | 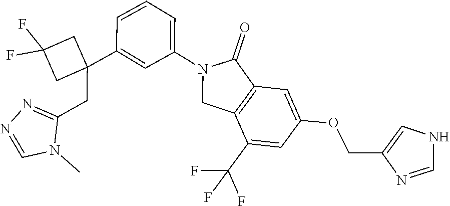 [SMILES: Cn1cnnc1CC1(c2cccc(N3Cc4c(cc(OCc5c[nH]cn5)cc4C(F)(F)F)C3=O)c2)CC(F)(F)C1] | 6-((1H-imidazol-4-yl)methoxy)-2-(3- (3,3-difluoro-1-((4-methyl-4H-1,2,4- triazol-3-yl)methyl)cyclobutyl)phenyl)-4- (trifluoromethyl)isoindolin-1-one |
| 423 | 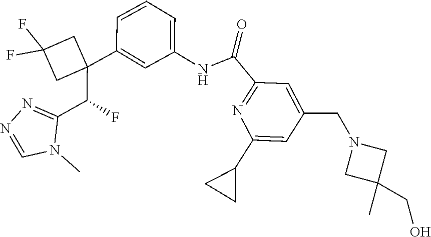 [SMILES: Cn1cnnc1[C@@H](F)C1(c2cccc(NC(=O)c3cc(CN4CC(C)(CO)C4)cc(C4CC4)n3)c2)CC(F)(F)C1] | (S)-6-cyclopropyl-N-(3-(3,3-difluoro-1- (fluoro(4-methyl-4H-1,2,4-triazol-3- yl)methyl)cyclobutyl)phenyl)-4-((3- (hydroxymethyl)-3-methylazetidin-1- yl)methyl)picolinamide |
| 424 | 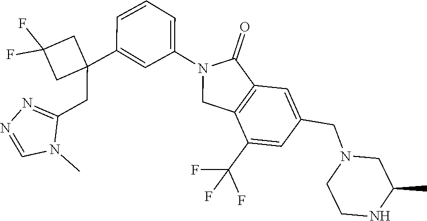 [SMILES: C[C@@H]1CN(Cc2cc3c(c(C(F)(F)F)c2)CN(c2cccc(C4(Cc5nncn5C)CC(F)(F)C4)c2)C3=O)CCN1] | (R)-2-(3-(3,3-difluoro-1-((4-methyl-4H- 1,2,4-triazol-3- yl)methyl)cyclobutyl)phenyl)-6-((3- methylpiperazin-1-yl)methyl)-4- (trifluoromethyl)isoindolin-1-one |
| 425 | 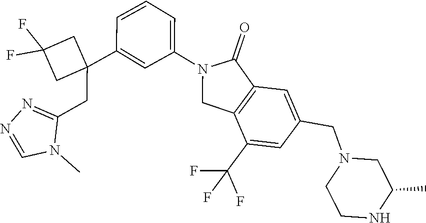 [SMILES: C[C@H]1CN(Cc2cc3c(c(C(F)(F)F)c2)CN(c2cccc(C4(Cc5nncn5C)CC(F)(F)C4)c2)C3=O)CCN1] | (S)-2-(3-(3,3-difluoro-1-((4-methyl-4H- 1,2,4-triazol-3- yl)methyl)cyclobutyl)phenyl)-6-((3- methylpiperazin-1-yl)methyl)-4- (trifluoromethyl)isoindolin-1-one |
| 426 | 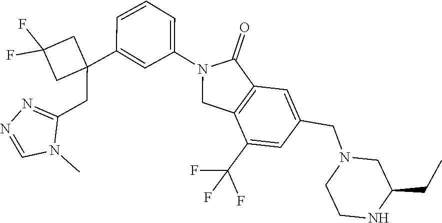 [SMILES: CC[C@@H]1CN(Cc2cc3c(c(C(F)(F)F)c2)CN(c2cccc(C4(Cc5nncn5C)CC(F)(F)C4)c2)C3=O)CCN1] | (R)-2-(3-(3,3-difluoro-1-((4-methyl-4H- 1,2,4-triazol-3- yl)methyl)cyclobutyl)phenyl)-6-((3- ethylpiperazin-1-yl)methyl)-4- (trifluoromethyl)isoindolin-1-one |
| 427 | 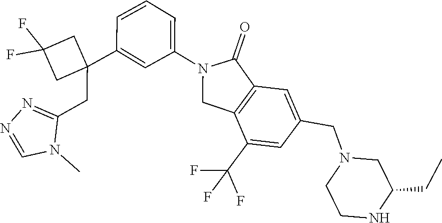 [SMILES: CC[C@H]1CN(Cc2cc3c(c(C(F)(F)F)c2)CN(c2cccc(C4(Cc5nncn5C)CC(F)(F)C4)c2)C3=O)CCN1] | (S)-2-(3-(3,3-difluoro-1-((4-methyl-4H- 1,2,4-triazol-3- yl)methyl)cyclobutyl)phenyl)-6-((3- ethylpiperazin-1-yl)methyl)-4- (trifluoromethyl)isoindolin-1-one |
| 428 | 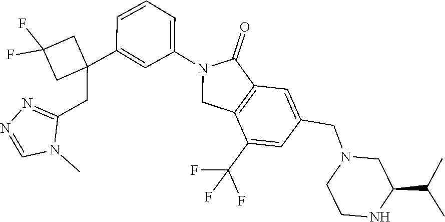 [SMILES: CC(C)[C@@H]1CN(Cc2cc3c(c(C(F)(F)F)c2)CN(c2cccc(C4(Cc5nncn5C)CC(F)(F)C4)c2)C3=O)CCN1] | (R)-2-(3-(3,3-difluoro-1-((4-methyl-4H- 1,2,4-triazol-3- yl)methyl)cyclobutyl)phenyl)-6-((3- isopropylpiperazin-1-yl)methyl)-4- (trifluoromethyl)isoindolin-1-one |
| 429 | 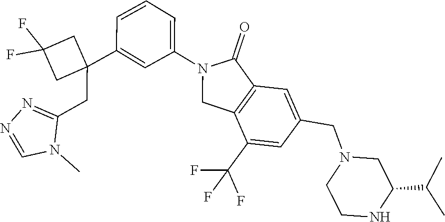 [SMILES: CC(C)[C@H]1CN(Cc2cc3c(c(C(F)(F)F)c2)CN(c2cccc(C4(Cc5nncn5C)CC(F)(F)C4)c2)C3=O)CCN1] | (S)-2-(3-(3,3-difluoro-1-((4-methyl-4H- 1,2,4-triazol-3- yl)methyl)cyclobutyl)phenyl)-6-((3- isopropylpiperazin-1-yl)methyl)-4- (trifluoromethyl)isoindolin-1-one |
| 430 | 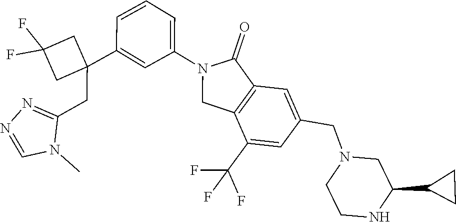 [SMILES: Cn1cnnc1CC1(c2cccc(N3Cc4c(cc(CN5CCN[C@H](C6CC6)C5)cc4C(F)(F)F)C3=O)c2)CC(F)(F)C1] | (R)-6-((3-cyclopropylpiperazin-1- yl)methyl)-2-(3-(3,3-difluoro-1-((4- methyl-4H-1,2,4-triazol-3- yl)methyl)cyclobutyl)phenyl)-4- (trifluoromethyl)isoindolin-1-one |
| 431 | 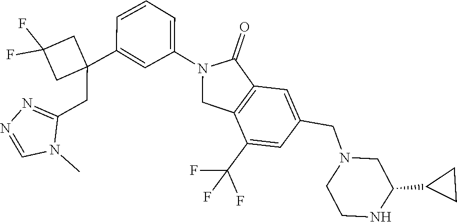 [SMILES: Cn1cnnc1CC1(c2cccc(N3Cc4c(cc(CN5CCN[C@@H](C6CC6)C5)cc4C(F)(F)F)C3=O)c2)CC(F)(F)C1] | (S)-6-((3-cyclopropylpiperazin-1- yl)methyl)-2-(3-(3,3-difluoro-1-((4- methyl-4H-1,2,4-triazol-3- yl)methyl)cyclobutyl)phenyl)-4- (trifluoromethyl)isoindolin-1-one |
| 432 | 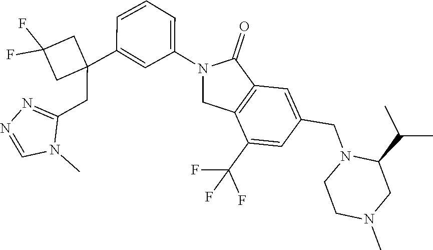 [SMILES: CC(C)[C@H]1CN(C)CCN1Cc1cc2c(c(C(F)(F)F)c1)CN(c1cccc(C3(Cc4nncn4C)CC(F)(F)C3)c1)C2=O] | (S)-2-(3-(1-(difluoro(4-methyl-4H-1,2,4- triazol-3-yl)methyl)-3,3- difluorocyclobutyl)phenyl)-6-((2- isopropyl-4-methylpiperazin-1- yl)methyl)-4-(trifluoromethyl)isoindolin- 1-one |
| 433 | 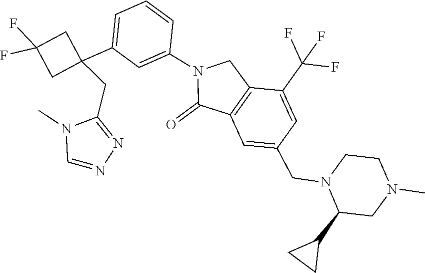 [SMILES: CN1CCN(Cc2cc3c(c(C(F)(F)F)c2)CN(c2cccc(C4(Cc5nncn5C)CC(F)(F)C4)c2)C3=O)[C@H](C2CC2)C1] | (R)-6-((2-cyclopropyl-4- methylpiperazin-1-yl)methyl)-2-(3-(3,3- difluoro-1-((4-methyl-4H-1,2,4-triazol-3- yl)methyl)cyclobutyl)phenyl)-4- (trifluoromethyl)isoindolin-1-one |
| 434 | 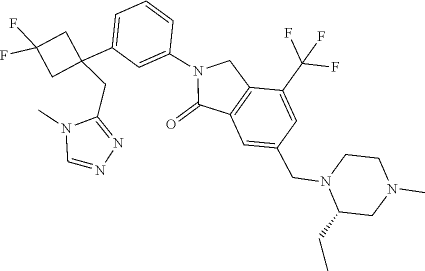 [SMILES: CC[C@H]1CN(C)CCN1Cc1cc2c(c(C(F)(F)F)c1)CN(c1cccc(C3(Cc4nncn4C)CC(F)(F)C3)c1)C2=O] | (S)-2-(3-(3,3-difluoro-1-((4-methyl-4H- 1,2,4-triazol-3- yl)methyl)cyclobutyl)phenyl)-6-((2- ethyl-4-methylpiperazin-1-yl)methyl)-4- (trifluoromethyl)isoindolin-1-one |
| 435 | 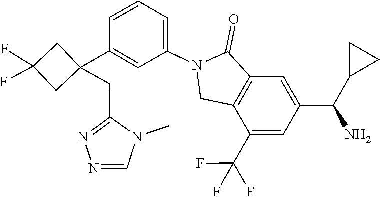 [SMILES: Cn1cnnc1CC1(c2cccc(N3Cc4c(cc([C@H](N)C5CC5)cc4C(F)(F)F)C3=O)c2)CC(F)(F)C1] | (R)-6-(amino(cyclopropyl)methyl)-2-(3- (3,3-difluoro-1-((4-methyl-4H-1,2,4- triazol-3-yl)methyl)cyclobutyl)phenyl)-4- (trifluoromethyl)isoindolin-1-one |
| 436 | 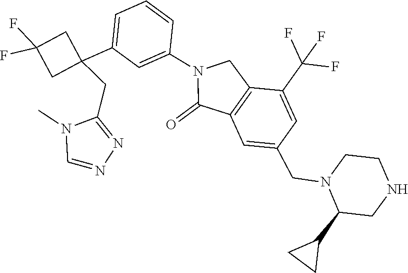 [SMILES: Cn1cnnc1CC1(c2cccc(N3Cc4c(cc(CN5CCNC[C@H]5C5CC5)cc4C(F)(F)F)C3=O)c2)CC(F)(F)C1] | (R)-6-((2-cyclopropylpiperazin-1- yl)methyl)-2-(3-(3,3-difluoro-1-((4- methyl-4H-1,2,4-triazol-3- yl)methyl)cyclobutyl)phenyl)-4- (trifluoromethyl)isoindolin-1-one |
| 437 | 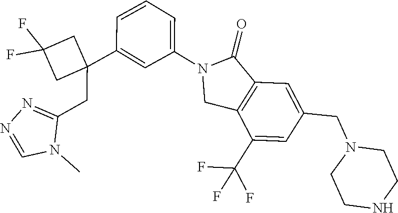 [SMILES: Cn1cnnc1CC1(c2cccc(N3Cc4c(cc(CN5CCNCC5)cc4C(F)(F)F)C3=O)c2)CC(F)(F)C1] | 2-(3-(3,3-difluoro-1-((4-methyl-4H- 1,2,4-triazol-3- yl)methyl)cyclobutyl)phenyl)-6- (piperazin-1-ylmethyl)-4- (trifluoromethyl)isoindolin-1-one |
| 438 | 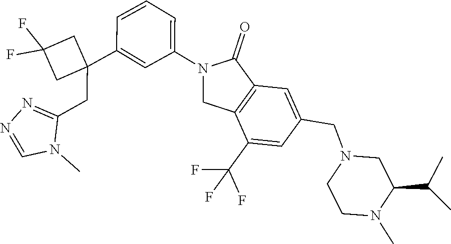 [SMILES: CC(C)[C@@H]1CN(Cc2cc3c(c(C(F)(F)F)c2)CN(c2cccc(C4(Cc5nncn5C)CC(F)(F)C4)c2)C3=O)CCN1C] | (R)-2-(3-(3,3-difluoro-1-((4-methyl-4H- 1,2,4-triazol-3- yl)methyl)cyclobutyl)phenyl)-6-((3- isopropyl-4-methylpiperazin-1- yl)methyl)-4-(trifluoromethyl)isoindolin- 1-one |
| 439 | 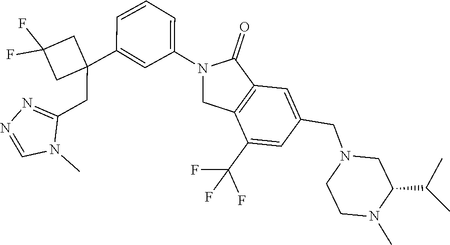 [SMILES: CC(C)[C@H]1CN(Cc2cc3c(c(C(F)(F)F)c2)CN(c2cccc(C4(Cc5nncn5C)CC(F)(F)C4)c2)C3=O)CCN1C] | (S)-2-(3-(3,3-difluoro-1-((4-methyl-4H- 1,2,4-triazol-3- yl)methyl)cyclobutyl)phenyl)-6-((3- isopropyl-4-methylpiperazin-1- yl)methyl)-4-(trifluoromethyl)isoindolin- 1-one |
| 440 | 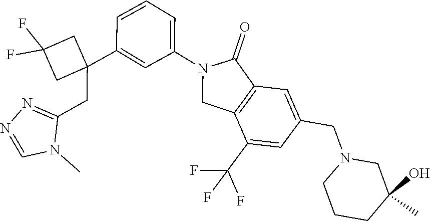 [SMILES: Cn1cnnc1CC1(c2cccc(N3Cc4c(cc(CN5CCC[C@](C)(O)C5)cc4C(F)(F)F)C3=O)c2)CC(F)(F)C1] | (S)-2-(3-(3,3-difluoro-1-((4-methyl-4H- 1,2,4-triazol-3- yl)methyl)cyclobutyl)phenyl)-6-((3- hydroxy-3-methylpiperidin-1-yl)methyl)- 4-(trifluoromethyl)isoindolin-1-one |
| 441 | 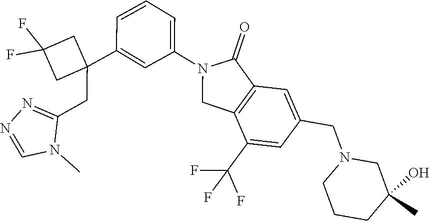 [SMILES: Cn1cnnc1CC1(c2cccc(N3Cc4c(cc(CN5CCC[C@@](C)(O)C5)cc4C(F)(F)F)C3=O)c2)CC(F)(F)C1] | (R)-2-(3-(3,3-difluoro-1-((4-methyl-4H- 1,2,4-triazol-3- yl)methyl)cyclobutyl)phenyl)-6-((3- hydroxy-3-methylpiperidin-1-yl)methyl)- 4-(trifluoromethyl)isoindolin-1-one |
| 442 | 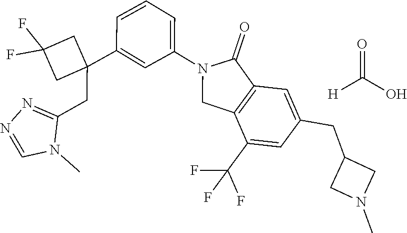 | 2-(3-(3,3-difluoro-1-((4-methyl-4H- 1,2,4-triazol-3- yl)methyl)cyclobutyl)phenyl)-6-((1- methylazetidin-3-yl)methyl)-4- (trifluoromethyl)isoindolin-1-one formate |
| 443 | 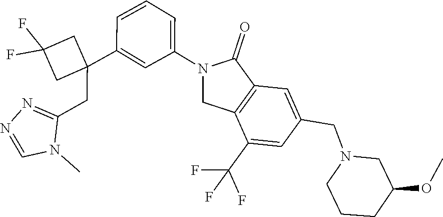 [SMILES: CO[C@H]1CCCN(Cc2cc3c(c(C(F)(F)F)c2)CN(c2cccc(C4(Cc5nncn5C)CC(F)(F)C4)c2)C3=O)C1] | (S)-2-(3-(3,3-difluoro-1-((4-methyl-4H- 1,2,4-triazol-3- yl)methyl)cyclobutyl)phenyl)-6-((3- methoxypiperidin-1-yl)methyl)-4- (trifluoromethyl)isoindolin-1-one |
| 444 | 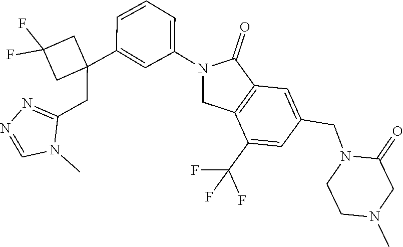 [SMILES: CN1CCN(Cc2cc3c(c(C(F)(F)F)c2)CN(c2cccc(C4(Cc5nncn5C)CC(F)(F)C4)c2)C3=O)C(=O)C1] | 2-(3-(3,3-difluoro-1-((4-methyl-4H- 1,2,4-triazol-3- yl)methyl)cyclobutyl)phenyl)-6-((4- methyl-2-oxopiperazin-1-yl)methyl)-4- (trifluoromethyl)isoindolin-1-one |
| 445 | 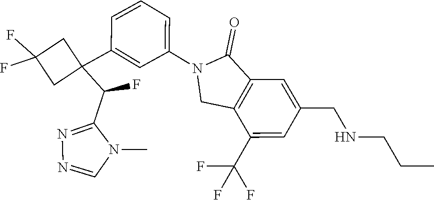 [SMILES: CCCNCc1cc2c(c(C(F)(F)F)c1)CN(c1cccc(C3([C@@H](F)c4nncn4C)CC(F)(F)C3)c1)C2=O] | (R)-2-(3-(3,3-difluoro-1-(fluoro(4- methyl-4H-1,2,4-triazol-3- yl)methyl)cyclobutyl)phenyl)-6- ((propylamino)methyl)-4- (trifluoromethyl)isoindolin-1-one |
| 446 | 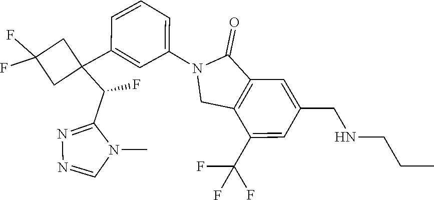 [SMILES: CCCNCc1cc2c(c(C(F)(F)F)c1)CN(c1cccc(C3([C@H](F)c4nncn4C)CC(F)(F)C3)c1)C2=O] | (S)-2-(3-(3,3-difluoro-1-(fluoro(4- methyl-4H-1,2,4-triazol-3- yl)methyl)cyclobutyl)phenyl)-6- ((propylamino)methyl)-4- (trifluoromethyl)isoindolin-1-one |
| 447 | 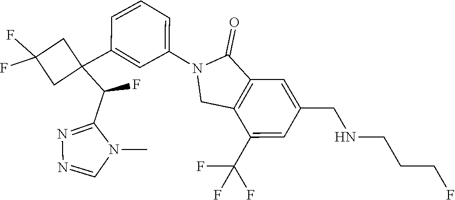 [SMILES: Cn1cnnc1[C@H](F)C1(c2cccc(N3Cc4c(cc(CNCCCF)cc4C(F)(F)F)C3=O)c2)CC(F)(F)C1] | (R)-2-(3-(3,3-difluoro-1-(fluoro(4- methyl-4H-1,2,4-triazol-3- yl)methyl)cyclobutyl)phenyl)-6-(((3- fluoropropyl)amino)methyl)-4- (trifluoromethyl)isoindolin-1-one |
| 448 | 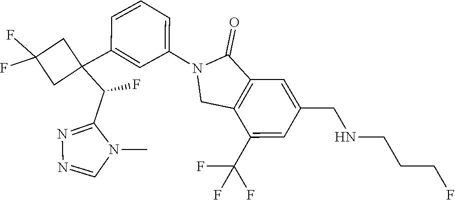 [SMILES: Cn1cnnc1[C@@H](F)C1(c2cccc(N3Cc4c(cc(CNCCCF)cc4C(F)(F)F)C3=O)c2)CC(F)(F)C1] | (S)-2-(3-(3,3-difluoro-1-(fluoro(4- methyl-4H-1,2,4-triazol-3- yl)methyl)cyclobutyl)phenyl)-6-(((3- fluoropropyl)amino)methyl)-4- (trifluoromethyl)isoindolin-1-one |
| 449 | 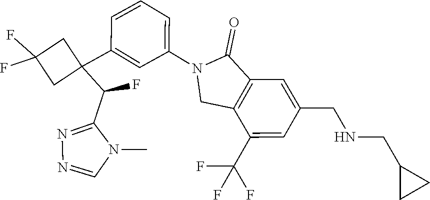 [SMILES: Cn1cnnc1[C@H](F)C1(c2cccc(N3Cc4c(cc(CNCC5CC5)cc4C(F)(F)F)C3=O)c2)CC(F)(F)C1] | (R)-6- (((cyclopropylmethyl)amino)methyl)-2- (3-(3,3-difluoro-1-(fluoro(4-methyl-4H- 1,2,4-triazol-3- yl)methyl)cyclobutyl)phenyl)-4- (trifluoromethyl)isoindolin-1-one |
| 450 | 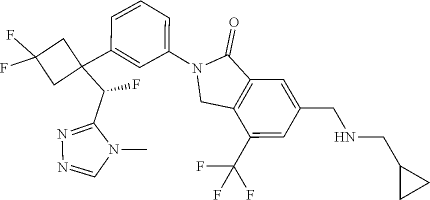 [SMILES: Cn1cnnc1[C@@H](F)C1(c2cccc(N3Cc4c(cc(CNCC5CC5)cc4C(F)(F)F)C3=O)c2)CC(F)(F)C1] | (S)-6- (((cyclopropylmethyl)amino)methyl)-2- (3-(3,3-difluoro-1-(fluoro(4-methyl-4H- 1,2,4-triazol-3- yl)methyl)cyclobutyl)phenyl)-4- (trifluoromethyl)isoindolin-1-one |
| 451 | 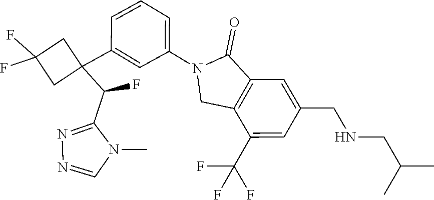 [SMILES: CC(C)CNCc1cc2c(c(C(F)(F)F)c1)CN(c1cccc(C3([C@@H](F)c4nncn4C)CC(F)(F)C3)c1)C2=O] | (R)-2-(3-(3,3-difluoro-1-(fluoro(4- methyl-4H-1,2,4-triazol-3- yl)methyl)cyclobutyl)phenyl)-6- ((isobutylamino)methyl)-4- (trifluoromethyl)isoindolin-1-one |
| 452 | 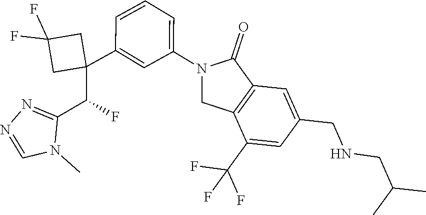 [SMILES: CC(C)CNCc1cc2c(c(C(F)(F)F)c1)CN(c1cccc(C3([C@H](F)c4nncn4C)CC(F)(F)C3)c1)C2=O] | (S)-2-(3-(3,3-difluoro-1-(fluoro(4- methyl-4H-1,2,4-triazol-3- yl)methyl)cyclobutyl)phenyl)-6- ((isobutylamino)methyl)-4- (trifluoromethyl)isoindolin-1-one |
| 453 | 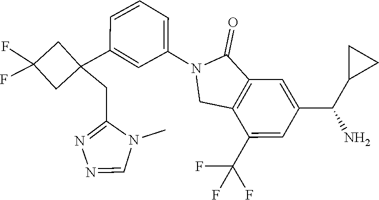 [SMILES: Cn1cnnc1CC1(c2cccc(N3Cc4c(cc([C@@H](N)C5CC5)cc4C(F)(F)F)C3=O)c2)CC(F)(F)C1] | (S)-6-(amino(cyclopropyl)methyl)-2-(3- (3,3-difluoro-1-((4-methyl-4H-1,2,4- triazol-3-yl)methyl)cyclobutyl)phenyl)-4- (trifluoromethyl)isoindolin-1-one |
| 454 | 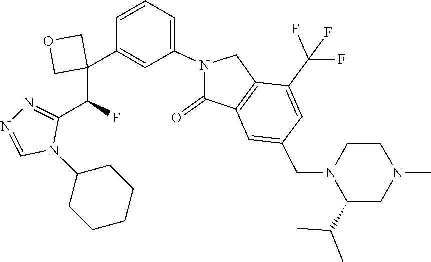 [SMILES: CC(C)[C@H]1CN(C)CCN1Cc1cc2c(c(C(F)(F)F)c1)CN(c1cccc(C3([C@@H](F)c4nncn4C4CCCCC4)COC3)c1)C2=O] | 2-(3-(3-((R)-(4-cyclohexyl-4H-1,2,4- triazol-3-yl)fluoromethyl)oxetan-3- yl)phenyl)-6-(((S)-2-isopropyl-4- methylpiperazin-1-yl)methyl)-4- (trifluoromethyl)isoindolin-1-one |
| 455 | 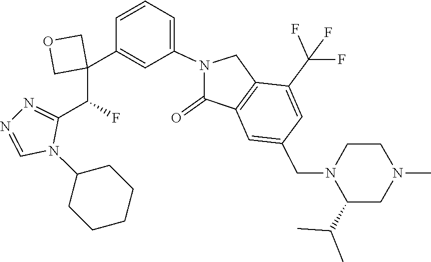 [SMILES: CC(C)[C@H]1CN(C)CCN1Cc1cc2c(c(C(F)(F)F)c1)CN(c1cccc(C3([C@H](F)c4nncn4C4CCCCC4)COC3)c1)C2=O] | 2-(3-(3-((S)-(4-cyclohexyl-4H-1,2,4- triazol-3-yl)fluoromethyl)oxetan-3- yl)phenyl)-6-(((S)-2-isopropyl-4- methylpiperazin-1-yl)methyl)-4- (trifluoromethyl)isoindolin-1-one |
| 456 | 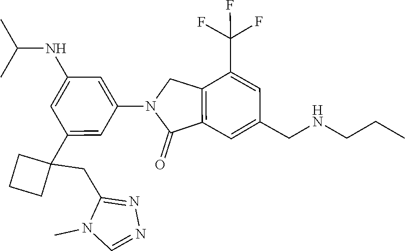 [SMILES: CCCNCc1cc2c(c(C(F)(F)F)c1)CN(c1cc(NC(C)C)cc(C3(Cc4nncn4C)CCC3)c1)C2=O] | 2-(3-(isopropylamino)-5-(1-((4-methyl- 4H-1,2,4-triazol-3- yl)methyl)cyclobutyl)phenyl)-6- ((propylamino)methyl)-4- (trifluoromethyl)isoindolin-1-one |
| 457 | 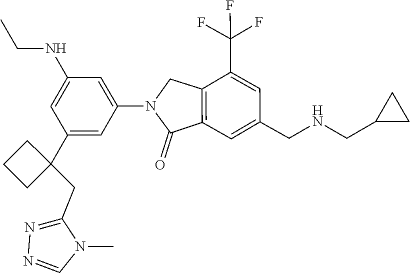 [SMILES: CCNc1cc(N2Cc3c(cc(CNCC4CC4)cc3C(F)(F)F)C2=O)cc(C2(Cc3nncn3C)CCC2)c1] | 6-(((cyclopropylmethyl)amino)methyl)- 2-(3-(ethylamino)-5-(1-((4-methyl-4H- 1,2,4-triazol-3- yl)methyl)cyclobutyl)phenyl)-4- (trifluoromethyl)isoindolin-1-one |
| 458 | 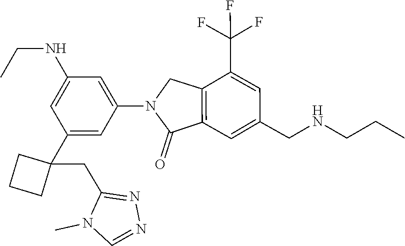 [SMILES: CCCNCc1cc2c(c(C(F)(F)F)c1)CN(c1cc(NCC)cc(C3(Cc4nncn4C)CCC3)c1)C2=O] | 2-(3-(ethylamino)-5-(1-((4-methyl-4H- 1,2,4-triazol-3- yl)methyl)cyclobutyl)phenyl)-6- ((propylamino)methyl)-4- (trifluoromethyl)isoindolin-1-one |
| 459 | 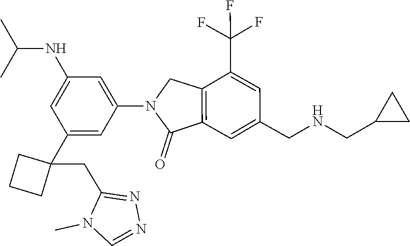 [SMILES: CC(C)Nc1cc(N2Cc3c(cc(CNCC4CC4)cc3C(F)(F)F)C2=O)cc(C2(Cc3nncn3C)CCC2)c1] | 6-(((cyclopropylmethyl)amino)methyl)- 2-(3-(isopropylamino)-5-(1-((4-methyl- 4H-1,2,4-triazol-3- yl)methyl)cyclobutyl)phenyl)-4- (trifluoromethyl)isoindolin-1-one |
| 460 | 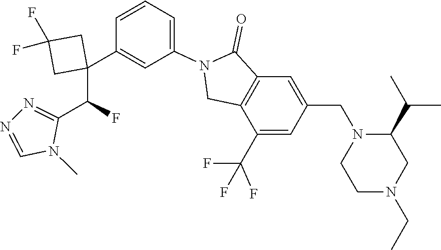 [SMILES: CCN1CCN(Cc2cc3c(c(C(F)(F)F)c2)CN(c2cccc(C4([C@@H](F)c5nncn5C)CC(F)(F)C4)c2)C3=O)[C@@H](C(C)C)C1] | 2-(3-(3,3-difluoro-1-((R)-fluoro(4- methyl-4H-1,2,4-triazol-3- yl)methyl)cyclobutyl)phenyl)-6-(((S)-4- ethyl-2-isopropylpiperazin-1-yl)methyl)- 4-(trifluoromethyl)isoindolin-1-one |
| 461 | 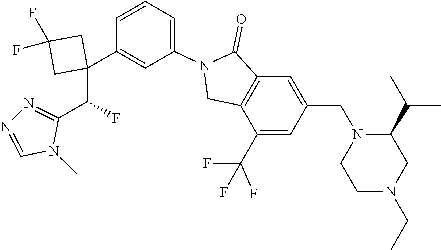 [SMILES: CCN1CCN(Cc2cc3c(c(C(F)(F)F)c2)CN(c2cccc(C4([C@H](F)c5nncn5C)CC(F)(F)C4)c2)C3=O)[C@@H](C(C)C)C1] | 2-(3-(3,3-difluoro-1-((S)-fluoro(4- methyl-4H-1,2,4-triazol-3- yl)methyl)cyclobutyl)phenyl)-6-(((S)-4- ethyl-2-isopropylpiperazin-1-yl)methyl)- 4-(trifluoromethyl)isoindolin-1-one |
| 462 | 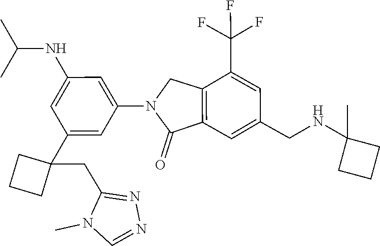 [SMILES: CC(C)Nc1cc(N2Cc3c(cc(CNC4(C)CCC4)cc3C(F)(F)F)C2=O)cc(C2(Cc3nncn3C)CCC2)c1] | 2-(3-(isopropylamino)-5-(1-((4-methyl- 4H-1,2,4-triazol-3- yl)methyl)cyclobutyl)phenyl)-6-(((1- methylcyclobutyl)amino)methyl)-4- (trifluoromethyl)isoindolin-1-one |
| 463 | 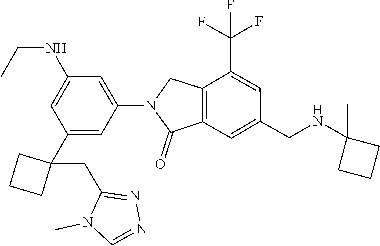 [SMILES: CCNc1cc(N2Cc3c(cc(CNC4(C)CCC4)cc3C(F)(F)F)C2=O)cc(C2(Cc3nncn3C)CCC2)c1] | 2-(3-(ethylamino)-5-(1-((4-methyl-4H- 1,2,4-triazol-3- yl)methyl)cyclobutyl)phenyl)-6-(((1- methylcyclobutyl)amino)methyl)-4- (trifluoromethyl)isoindolin-1-one |
| 464 | 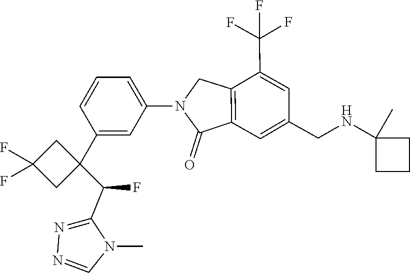 [SMILES: Cn1cnnc1[C@H](F)C1(c2cccc(N3Cc4c(cc(CNC5(C)CCC5)cc4C(F)(F)F)C3=O)c2)CC(F)(F)C1] | (R)-2-(3-(3,3-difluoro-1-(fluoro(4- methyl-4H-1,2,4-triazol-3- yl)methyl)cyclobutyl)phenyl)-6-(((1- methylcyclobutyl)amino)methyl)-4- (trifluoromethyl)isoindolin-1-one |
| 465 | 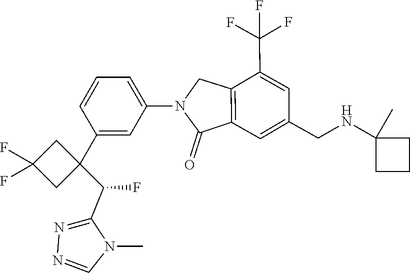 [SMILES: Cn1cnnc1[C@@H](F)C1(c2cccc(N3Cc4c(cc(CNC5(C)CCC5)cc4C(F)(F)F)C3=O)c2)CC(F)(F)C1] | (S)-2-(3-(3,3-difluoro-1-(fluoro(4- methyl-4H-1,2,4-triazol-3- yl)methyl)cyclobutyl)phenyl)-6-(((1- methylcyclobutyl)amino)methyl)-4- (trifluoromethyl)isoindolin-1-one |
| 466 | 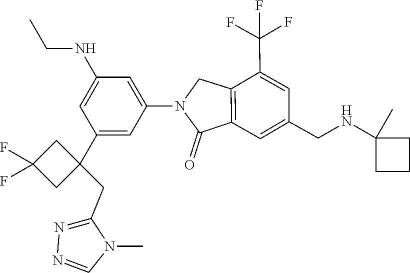 [SMILES: CCNc1cc(N2Cc3c(cc(CNC4(C)CCC4)cc3C(F)(F)F)C2=O)cc(C2(Cc3nncn3C)CC(F)(F)C2)c1] | 2-(3-(3,3-difluoro-1-((4-methyl-4H- 1,2,4-triazol-3-yl)methyl)cyclobutyl)-5- (ethylamino)phenyl)-6-(((1- methylcyclobutyl)amino)methyl)-4- (trifluoromethyl)isoindolin-1-one |
| 467 | 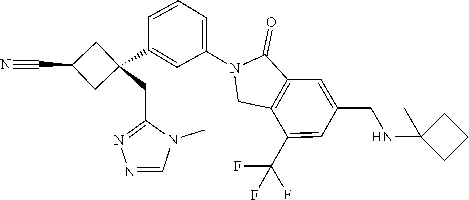 [SMILES: Cn1cnnc1C[C@]1(c2cccc(N3Cc4c(cc(CNC5(C)CCC5)cc4C(F)(F)F)C3=O)c2)C[C@H](C#N)C1] | (1r,3r)-3-((4-methyl-4H-1,2,4-triazol-3- yl)methyl)-3-(3-(6-(((1- methylcyclobutyl)amino)methyl)-1-oxo- 4-(trifluoromethyl)isoindolin-2- yl)phenyl)cyclobutanecarbonitrile |
| 468 | 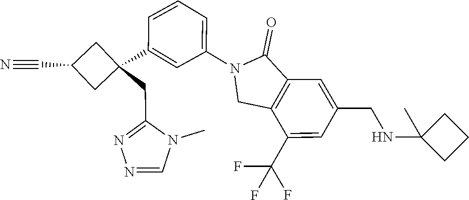 [SMILES: Cn1cnnc1C[C@]1(c2cccc(N3Cc4c(cc(CNC5(C)CCC5)cc4C(F)(F)F)C3=O)c2)C[C@@H](C#N)C1] | (1s,3s)-3-((4-methyl-4H-1,2,4-triazol-3- yl)methyl)-3-(3-(6-(((1 - methylcyclobutyl)amino)methyl)-1-oxo- 4-(trifluoromethyl)isoindolin-2- yl)phenyl)cyclobutanecarbonitrile |
| 469 | 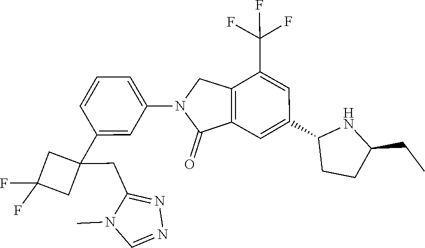 [SMILES: CC[C@H]1CC[C@H](c2cc3c(c(C(F)(F)F)c2)CN(c2cccc(C4(Cc5nncn5C)CC(F)(F)C4)c2)C3=O)N1] | 2-(3-(3,3-difluoro-1-((4-methyl-4H- 1,2,4-triazol-3- yl)methyl)cyclobutyl)phenyl)-6- ((2R,5S)-5-ethylpyrrolidin-2-yl)-4- (trifluoromethyl)isoindolin-1-one |
| 470 | 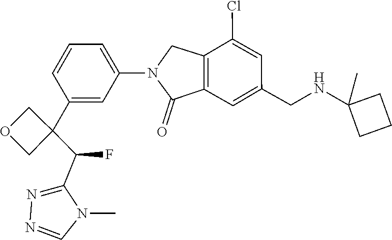 [SMILES: Cn1cnnc1[C@H](F)C1(c2cccc(N3Cc4c(Cl)cc(CNC5(C)CCC5)cc4C3=O)c2)COC1] | (R)-4-chloro-2-(3-(3-(fluoro(4-methyl- 4H-1,2,4-triazol-3-yl)methyl)oxetan-3- yl)phenyl)-6-(((1- methylcyclobutyl)amino)methyl) isoindolin-1-one |
| 471 | 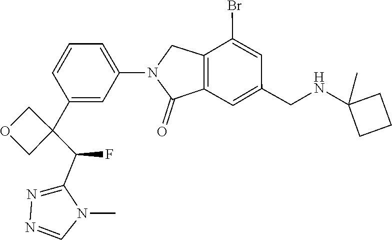 [SMILES: Cn1cnnc1[C@H](F)C1(c2cccc(N3Cc4c(Br)cc(CNC5(C)CCC5)cc4C3=O)c2)COC1] | (R)-4-bromo-2-(3-(3-(fluoro(4-methyl- 4H-1,2,4-triazol-3-yl)methyl)oxetan-3- yl)phenyl)-6-(((1- methylcyclobutyl)amino)methyl) isoindolin-1-one |
| 472 | 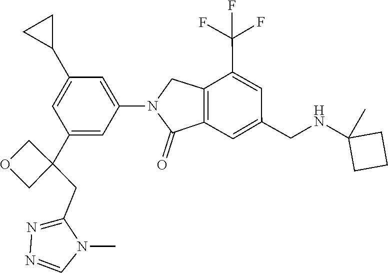 [SMILES: Cn1cnnc1CC1(c2cc(C3CC3)cc(N3Cc4c(cc(CNC5(C)CCC5)cc4C(F)(F)F)C3=O)c2)COC1] | 2-(3-cyclopropyl-5-(3-((4-methyl-4H- 1,2,4-triazol-3-yl)methyl)oxetan-3- yl)phenyl)-6-(((1- methylcyclobutyl)amino)methyl)-4- (trifluoromethyl)isoindolin-1-one |
| 473 | 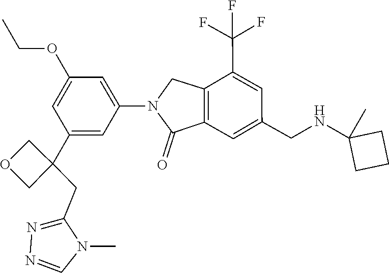 [SMILES: CCOc1cc(N2Cc3c(cc(CNC4(C)CCC4)cc3C(F)(F)F)C2=O)cc(C2(Cc3nncn3C)COC2)c1] | 2-(3-ethoxy-5-(3-((4-methyl-4H-1,2,4- triazol-3-yl)methyl)oxetan-3-yl)phenyl)- 6-(((1-methylcyclobutyl)amino)methyl)- 4-(trifluoromethyl)isoindolin-1-one |
| 474 | 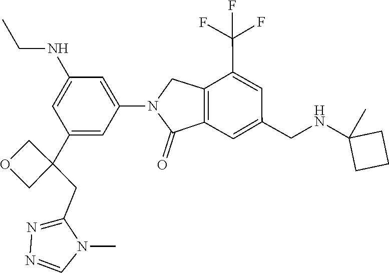 [SMILES: CCNc1cc(N2Cc3c(cc(CNC4(C)CCC4)cc3C(F)(F)F)C2=O)cc(C2(Cc3nncn3C)COC2)c1] | 2-(3-(ethylamino)-5-(3-((4-methyl-4H- 1,2,4-triazol-3-yl)methyl)oxetan-3- yl)phenyl)-6-(((1- methylcyclobutyl)amino)methyl)-4- (trifluoromethyl)isoindolin-1-one |
| 475 | 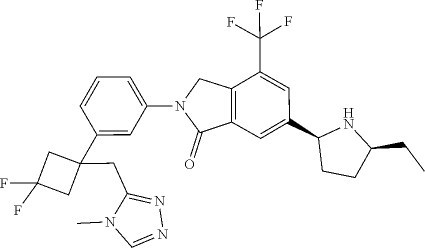 [SMILES: CC[C@H]1CC[C@@H](c2cc3c(c(C(F)(F)F)c2)CN(c2cccc(C4(Cc5nncn5C)CC(F)(F)C4)c2)C3=O)N1] | 2-(3-(3,3-difluoro-1-((4-methyl-4H- 1,2,4-triazol-3- yl)methyl)cyclobutyl)phenyl)-6- ((2S,5S)-5-ethylpyrrolidin-2-yl)-4- (trifluoromethyl)isoindolin-1-one |
| 476 | 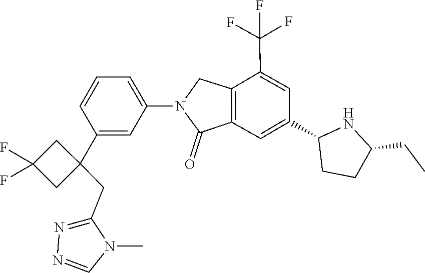 [SMILES: CC[C@@H]1CC[C@H](c2cc3c(c(C(F)(F)F)c2)CN(c2cccc(C4(Cc5nncn5C)CC(F)(F)C4)c2)C3=O)N1] | 2-(3-(3,3-difluoro-1-((4-methyl-4H- 1,2,4-triazol-3- yl)methyl)cyclobutyl)phenyl)-6- ((2R,5R)-5-ethylpyrrolidin-2-yl)-4- (trifluoromethyl)isoindolin-1-one |
| 477 | 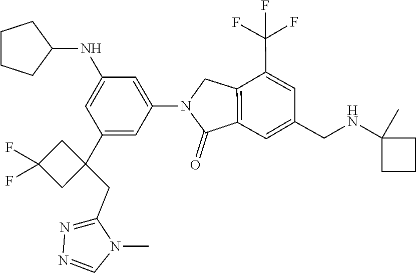 [SMILES: Cn1cnnc1CC1(c2cc(NC3CCCC3)cc(N3Cc4c(cc(CNC5(C)CCC5)cc4C(F)(F)F)C3=O)c2)CC(F)(F)C1] | 2-(3-(cyclopentylamino)-5-(3,3-difluoro- 1-((4-methyl-4H-1,2,4-triazol-3- yl)methyl)cyclobutyl)phenyl)-6-(((1- methylcyclobutyl)amino)methyl)-4- (trifluoromethyl)isoindolin-1-one |
| 478 | 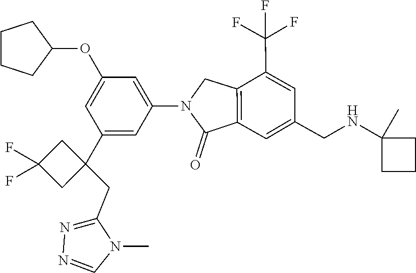 [SMILES: Cn1cnnc1CC1(c2cc(OC3CCCC3)cc(N3Cc4c(cc(CNC5(C)CCC5)cc4C(F)(F)F)C3=O)c2)CC(F)(F)C1] | 2-(3-(cyclopentyloxy)-5-(3,3-difluoro-1- ((4-methyl-4H-1,2,4-triazol-3- yl)methyl)cyclobutyl)phenyl)-6-(((1- methylcyclobutyl)amino)methyl)-4- (trifluoromethyl)isoindolin-1-one |
| 479 | 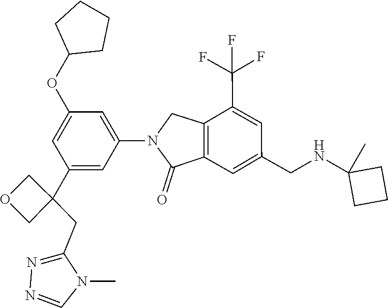 [SMILES: Cn1cnnc1CC1(c2cc(OC3CCCC3)cc(N3Cc4c(cc(CNC5(C)CCC5)cc4C(F)(F)F)C3=O)c2)COC1] | 2-(3-(cyclopentyloxy)-5-(3-((4-methyl- 4H-1,2,4-triazol-3-yl)methyl)oxetan-3- yl)phenyl)-6-(((1- methylcyclobutyl)amino)methyl)-4- (trifluoromethyl)isoindolin-1-one |
| 480 | 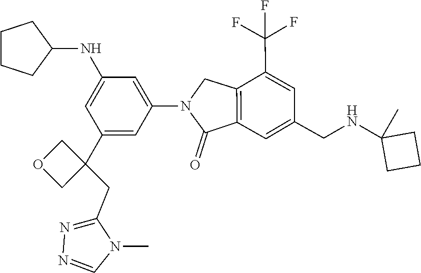 [SMILES: Cn1cnnc1CC1(c2cc(NC3CCCC3)cc(N3Cc4c(cc(CNC5(C)CCC5)cc4C(F)(F)F)C3=O)c2)COC1] | 2-(3-(cyclopentylamino)-5-(3-((4- methyl-4H-1,2,4-triazol-3- yl)methyl)oxetan-3-yl)phenyl)-6-(((1- methylcyclobutyl)amino)methyl)-4- (trifluoromethyl)isoindolin-1-one |
| 481 | 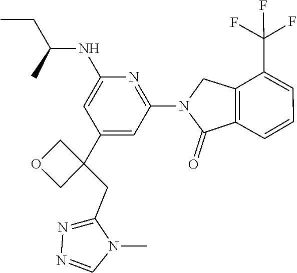 [SMILES: CC[C@H](C)Nc1cc(C2(Cc3nncn3C)COC2)cc(N2Cc3c(cccc3C(F)(F)F)C2=O)n1] | (S)-2-(6-(sec-butylamino)-4-(3-((4- methyl-4H-1,2,4-triazol-3- yl)methyl)oxetan-3-yl)pyridin-2-yl)-4- (trifluoromethyl)isoindolin-1-one |
| 482 | 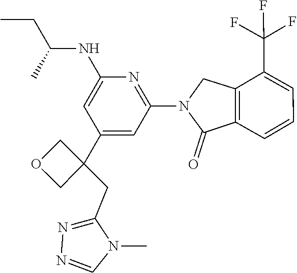 [SMILES: CC[C@@H](C)Nc1cc(C2(Cc3nncn3C)COC2)cc(N2Cc3c(cccc3C(F)(F)F)C2=O)n1] | (R)-2-(6-(sec-butylamino)-4-(3-((4- methyl-4H-1,2,4-triazol-3- yl)methyl)oxetan-3-yl)pyridin-2-yl)-4- (trifluoromethyl)isoindolin-1-one |
| 483 | 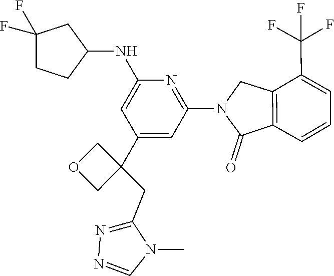 [SMILES: Cn1cnnc1CC1(c2cc(NC3CCC(F)(F)C3)nc(N3Cc4c(cccc4C(F)(F)F)C3=O)c2)COC1] | 2-(6-((3,3-difluorocyclopentyl)amino)-4- (3-((4-methyl-4H-1,2,4-triazol-3- yl)methyl)oxetan-3-yl)pyridin-2-yl)-4- (trifluoromethyl)isoindolin-1-one |
| 484 | 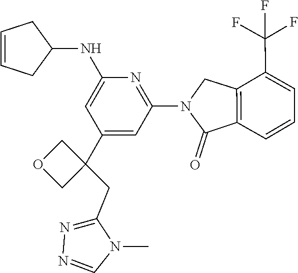 [SMILES: Cn1cnnc1CC1(c2cc(NC3CC=CC3)nc(N3Cc4c(cccc4C(F)(F)F)C3=O)c2)COC1] | 2-(6-(cyclopent-3-en-1-ylamino)-4-(3- ((4-methyl-4H-1,2,4-triazol-3- yl)methyl)oxetan-3-yl)pyridin-2-yl)-4- (trifluoromethyl)isoindolin-1-one |
| 485 | 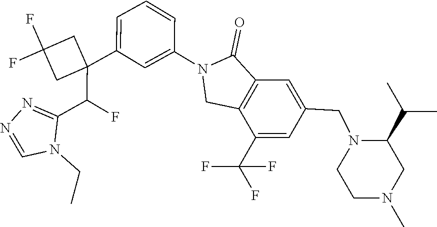 [SMILES: CCn1cnnc1C(F)C1(c2cccc(N3Cc4c(cc(CN5CCN(C)C[C@@H]5C(C)C)cc4C(F)(F)F)C3=O)c2)CC(F)(F)C1] | 2-(3-(1-((4-ethyl-4H-1,2,4-triazol-3- yl)fluoromethyl)-3,3- difluorocyclobutyl)phenyl)-6-(((S)-2- isopropyl-4-methylpiperazin-1- yl)methyl)-4-(trifluoromethyl) isoindolin-1-one |
| 486 | 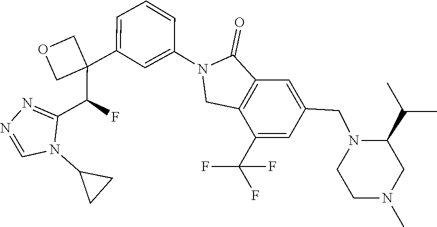 [SMILES: CC(C)[C@H]1CN(C)CCN1Cc1cc2c(c(C(F)(F)F)c1)CN(c1cccc(C3([C@@H](F)c4nncn4C4CC4)COC3)c1)C2=O] | 2-(3-(3-((R)-(4-cyclopropyl-4H-1,2,4- triazol-3-yl)fluoromethyl)oxetan-3- yl)phenyl)-6-(((S)-2-isopropyl-4- methylpiperazin-1-yl)methyl)-4- (trifluoromethyl)isoindolin-1-one |
| 487 | 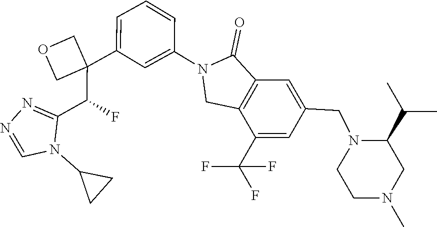 [SMILES: CC(C)[C@H]1CN(C)CCN1Cc1cc2c(c(C(F)(F)F)c1)CN(c1cccc(C3([C@H](F)c4nncn4C4CC4)COC3)c1)C2=O] | 2-(3-(3-((S)-(4-cyclopropyl-4H-1,2,4- triazol-3-yl)fluoromethyl)oxetan-3- yl)phenyl)-6-(((S)-2-isopropyl-4- methylpiperazin-1-yl)methyl)-4- (trifluoromethyl)isoindolin-1-one |
| 488 | 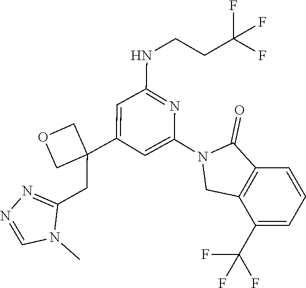 [SMILES: Cn1cnnc1CC1(c2cc(NCCC(F)(F)F)nc(N3Cc4c(cccc4C(F)(F)F)C3=O)c2)COC1] | 2-(4-(3-((4-methyl-4H-1,2,4-triazol-3- yl)methyl)oxetan-3-yl)-6-((3,3,3- trifluoropropyl)amino)pyridin-2-yl)-4- (trifluoromethyl)isoindolin-1-one |
| 489 | 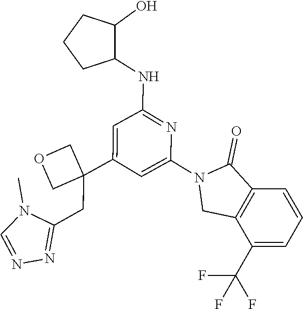 [SMILES: Cn1cnnc1CC1(c2cc(NC3CCCC3O)nc(N3Cc4c(cccc4C(F)(F)F)C3=O)c2)COC1] | 2-(6-((2-hydroxycyclopentyl)amino)-4- (3-((4-methyl-4H-1,2,4-triazol-3- yl)methyl)oxetan-3-yl)pyridin-2-yl)-4- (trifluoromethyl)isoindolin-1-one |
| 490 | 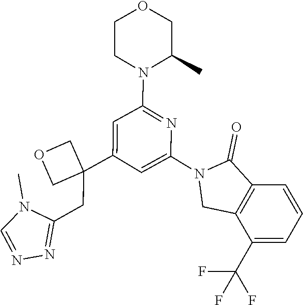 [SMILES: C[C@@H]1COCCN1c1cc(C2(Cc3nncn3C)COC2)cc(N2Cc3c(cccc3C(F)(F)F)C2=O)n1] | (R)-2-(4-(3-((4-methyl-4H-1,2,4-triazol- 3-yl)methyl)oxetan-3-yl)-6-(3- methylmorpholino)pyridin-2-yl)-4- (trifluoromethyl)isoindolin-1-one |
| 491 | 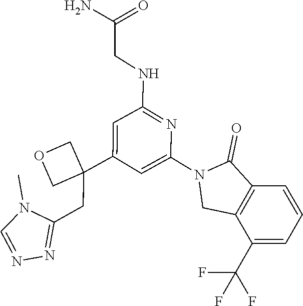 [SMILES: Cn1cnnc1CC1(c2cc(NCC(N)=O)nc(N3Cc4c(cccc4C(F)(F)F)C3=O)c2)COC1] | 2-((4-(3-((4-methyl-4H-1,2,4-triazol-3- yl)methyl)oxetan-3-yl)-6-(1-oxo-4- (trifluoromethyl)isoindolin-2-yl)pyridin- 2-yl)amino)acetamide |
| 492 | 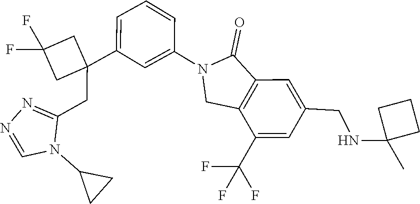 [SMILES: CC1(NCc2cc3c(c(C(F)(F)F)c2)CN(c2cccc(C4(Cc5nncn5C5CC5)CC(F)(F)C4)c2)C3=O)CCC1] | 2-(3-(1-((4-cyclopropyl-4H-1,2,4-triazol- 3-yl)methyl)-3,3- difluorocyclobutyl)phenyl)-6-(((1- methylcyclobutyl)amino)methyl)-4- (trifluoromethyl)isoindolin-1-one |
| 493 | 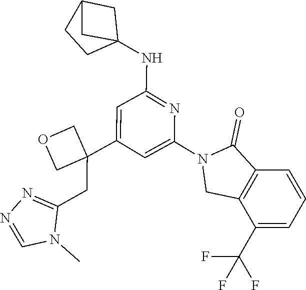 [SMILES: Cn1cnnc1CC1(c2cc(NC34CCC(C3)C4)nc(N3Cc4c(cccc4C(F)(F)F)C3=O)c2)COC1] | 2-(6-(bicyclo[2.1.1]hexan-1-ylamino)-4- (3-((4-methyl-4H-1,2,4-triazol-3- yl)methyl)oxetan-3-yl)pyridin-2-yl)-4- (trifluoromethyl)isoindolin-1-one |
| 494 | 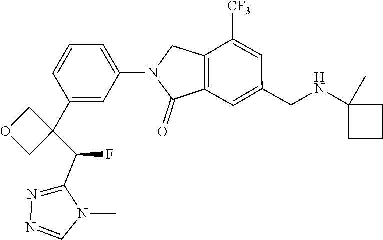 [SMILES: Cn1cnnc1[C@H](F)C1(c2cccc(N3Cc4c(cc(CNC5(C)CCC5)cc4C(F)(F)F)C3=O)c2)COC1] | (R)-2-(3-(3-(fluoro(4-methyl-4H-1,2,4- triazol-3-yl)methyl)oxetan-3-yl)phenyl)- 6-(((1-methylcyclobutyl)amino)methyl)- 4-(trifluoromethyl)isoindolin-1-one |

### Pharmaceutical Compositions and Formulations

The presently disclosed compounds can be formulated into pharmaceutical compositions along with a pharmaceutically acceptable carrier or excipient. According to this aspect, there is provided a pharmaceutical composition comprising a compound of Formulae (I-A)-(I-G) in association with a pharmaceutically acceptable excipient, diluent or carrier.

The formulations of Compounds of Formulae (I-A)-(I-G) include those suitable for the administration routes detailed herein. They may conveniently be presented in unit dosage form and can be formulated in accordance with standard pharmaceutical practice as a pharmaceutical composition. Techniques and formulations generally and suitable for use herein are found in Remington's Pharmaceutical Sciences (16^{th} edition, Osol, A. Ed. (1980); Mack Publishing Co., Easton, PA). Such methods include the step of bringing into association the active ingredient with the excipient or carrier which constitutes one or more accessory ingredients. In general the formulations are prepared by uniformly and intimately bringing into association the active ingredient with liquid excipients or carriers or finely divided solid excipients or carriers or both, and then, if necessary, shaping the product.

A typical formulation is prepared by mixing a compound of Formulae (I-A)-(I-G), and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water and the like. The particular carrier, diluent or excipient used will depend upon the means and purpose for which the compound of Formulae (I-A)-(I-G), is being applied. Solvents are generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of Formulae (I-A)-(I-G), or pharmaceutical composition thereof) or aid in the manufacturing of the pharmaceutical product (i.e., medicament).

The formulations may be prepared using conventional dissolution and mixing procedures. For example, the bulk drug substance (i.e., compound of Formulae (I-A)-(I-G), or stabilized form of the Compound of Formulae (I-A)-(I-G), (e.g., complex with a cyclodextrin derivative or other known complexation agent) is dissolved in a suitable solvent in the presence of one or more of the excipients described above. The compound of Formulae (I-A)-(I-G) is typically formulated into pharmaceutical dosage forms to provide an easily controllable dosage of the drug and to enable patient compliance with the prescribed regimen.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

Pharmaceutical formulations may be prepared for various routes and types of administration. For example, a compound of Formulae (I-A)-(I-G) having the desired degree of purity may optionally be mixed with pharmaceutically acceptable diluents, carriers, excipients or stabilizers (Remington's Pharmaceutical Sciences (1980 16^{th} edition, Osol, A. Ed.), in the form of a lyophilized formulation, milled powder, or an aqueous solution. Formulation may be conducted by mixing at ambient temperature at the appropriate pH, and at the desired degree of purity, with physiologically acceptable excipients or carriers, i.e., excipients or carriers that are non-toxic to recipients at the dosages and concentrations employed. The pH of the formulation depends mainly on the particular use and the concentration of compound, but may range from about 3 to about 8. Formulation in an acetate buffer at pH 5 is a suitable embodiment.

The compounds of Formulae (I-A)-(I-G) can be sterile. In particular, formulations to be used for in vivo administration should be sterile. Such sterilization is readily accomplished by filtration through sterile filtration membranes.

The compound of Formulae (I-A)-(I-G) ordinarily can be stored as a solid composition, a lyophilized formulation or as an aqueous solution.

The pharmaceutical compositions comprising a compound of Formulae (I-A)-(I-G) can be formulated, dosed and administered in a fashion, i.e., amounts, concentrations, schedules, course, vehicles and route of administration, consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the compound to be administered will be governed by such considerations, and is the minimum amount necessary to prevent, ameliorate, or treat the coagulation factor mediated disorder. In some embodiments, the amount is below the amount that is toxic to the host or renders the host more susceptible to bleeding.

Acceptable diluents, carriers, excipients and stabilizers are nontoxic to recipients at the dosages and concentrations employed, and include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™ PLURONICS™ or polyethylene glycol (PEG). The active pharmaceutical ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16^{th} edition, Osol, A. Ed. (1980).

Sustained-release preparations of Formulae (I-A)-(I-G) compounds may be prepared. Suitable examples of sustained-release preparations include semipermeable matrices of solid hydrophobic polymers containing a compound of Formulae (I-A)-(1-G), which matrices are in the form of shaped articles, e.g., films, or microcapsules. Examples of sustained-release matrices include polyesters, hydrogels (for example, poly(2-hydroxyethyl-methacrylate), or poly(vinyl alcohol)), polylactides, copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate) and poly-D-(−)-3-hydroxybutyric acid.

Formulations of a compound of Formulae (I-A)-(I-G) suitable for oral administration may be prepared as discrete units such as pills, capsules, cachets or tablets each containing a predetermined amount of a compound of Formulae (I-A)-(I-G).

Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

Tablets, troches, lozenges, aqueous or oil suspensions, dispersible powders or granules, emulsions, hard or soft capsules, e.g., gelatin capsules, syrups or elixirs may be prepared for oral use. Formulations of compounds of Formulae (I-A)-(I-G) intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

For treatment of the eye or other external tissues, e.g., mouth and skin, the formulations are preferably applied as a topical ointment or cream containing the active ingredient(s) in an amount of, for example, 0.075 to 20% w/w. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include a polyhydric alcohol, i.e., an alcohol having two or more hydroxyl groups such as propylene glycol, butane 1,3-diol, mannitol, sorbitol, glycerol and polyethylene glycol (including PEG 400), and mixtures thereof. The topical formulations may desirably include a compound which enhances absorption or penetration of the active ingredient through the skin or other affected areas. Examples of such dermal penetration enhancers include dimethyl sulfoxide and related analogs.

The oily phase of the emulsions may be constituted from known ingredients in a known manner. While the phase may comprise solely an emulsifier, it may also comprise a mixture of at least one emulsifier and a fat or oil, or both a fat and an oil. A hydrophilic emulsifier included together with a lipophilic emulsifier may act as a stabilizer. Together, the emulsifier(s) with or without stabilizer(s) make up the so-called emulsifying wax, and the wax together with the oil and fat make up the so-called emulsifying ointment base which forms the oily dispersed phase of the cream formulations. Emulsifiers and emulsion stabilizers suitable for use in the formulation include Tween® 60, Span® 80, cetostearyl alcohol, benzyl alcohol, myristyl alcohol, glyceryl mono-stearate and sodium lauryl sulfate.

Aqueous suspensions of Formulae (I-A)-(I-G) compounds contain the active materials in admixture with excipients suitable for the manufacture of aqueous suspensions. Such excipients include a suspending agent, such as sodium carboxymethylcellulose, croscarmellose, povidone, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxybenzoate, one or more coloring agents, one or more flavoring agents and one or more sweetening agents, such as sucrose or saccharin.

The pharmaceutical compositions of compounds of Formulae (I-A)-(I-G), may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such 1,3-butanediol. The sterile injectable preparation may also be prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the excipient or carrier material to produce a single dosage form will vary depending upon the host treated and the particular mode of administration. For example, a time-release formulation intended for oral administration to humans may contain approximately 1 to 1000 mg of active material compounded with an appropriate and convenient amount of excipient or carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

Formulations suitable for topical administration to the eye also include eye drops in which the active ingredient is dissolved or suspended in a suitable excipient or carrier, especially an aqueous solvent for the active ingredient. The active ingredient is preferably present in such formulations in a concentration of about 0.5 to 20% w/w, for example about 0.5 to 10% w/w, for example about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for intrapulmonary or nasal administration have a particle size for example in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis of disorders as described below.

Formulations suitable for vaginal administration may be presented as pessaries, tampons, creams, gels, pastes, foams or spray formulations containing in addition to the active ingredient such excipients or carriers as are known in the art to be appropriate.

The formulations may be packaged in unit-dose or multi-dose containers, for example sealed ampoules and vials, and may be stored in a freeze-dried (lyophilized) condition requiring only the addition of the sterile liquid excipient or carrier, for example water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

The subject matter further provides veterinary compositions comprising at least one active ingredient as above defined together with a veterinary excipient or carrier therefore. Veterinary excipients or carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

In particular embodiments the pharmaceutical composition comprising the presently disclosed compounds further comprise a chemotherapeutic agent. In some of these embodiments, the chemotherapeutic agent is an immunotherapeutic agent.

### Kits

Further provided are kits for carrying out the methods detailed herein, which kits comprise one or more compounds described herein or a pharmaceutical composition comprising a compound described herein. The kits may employ any of the compounds disclosed herein. In one variation, the kit employs a compound described herein or a pharmaceutically acceptable salt thereof. The kits may be used for any one or more of the uses described herein, and, accordingly, may contain instructions for use in the treatment of a disorder such as cancer. In some embodiments, the kit contains instructions for use in the treatment of a cancer.

Kits generally comprise suitable packaging. The kits may comprise one or more containers comprising any compound described herein. Each component (if there is more than one component) can be packaged in separate containers or some components can be combined in one container where cross-reactivity and shelf life permit. One or more components of a kit may be sterile and/or may be contained within sterile packaging.

The kits may be in unit dosage forms, bulk packages (e.g., multi-dose packages) or sub-unit doses. For example, kits may be provided that contain sufficient dosages of a compound as disclosed herein (e.g., a therapeutically effective amount) and/or a second pharmaceutically active compound useful for a disorder (e.g., cancer) to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and may be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present invention. The instructions included with the kit generally include information as to the components and their administration to a subject.

### Methods of Use

The presently disclosed compounds find use in inhibiting the activity of Cbl-B. Many of the compounds additionally do so with an inhibitory effect that is greater than that for C-cbl.

In one embodiment, the subject matter disclosed herein is directed to a method of inhibiting Cbl-B, the method comprising contacting one or more cells containing active Cbl-B proteins with an effective amount of a compound of Formulae (I-A)-(I-G), or a pharmaceutical composition described herein. By “contacting” is meant bringing the compound within close enough proximity to an isolated Cbl-B enzyme or a cell expressing Cbl-B (e.g., T cell, B cell, dendritic cell) such that the compound is able to bind to and inhibit the activity of Cbl-B. The compound can be contacted with Cbl-B in vitro or in vivo via administration of the compound to a subject.

In an embodiment, the subject matter disclosed herein is directed to a method for enhancing an immune response in a subject in need thereof, wherein the method comprises administering to said subject an effective amount of a compound of Formulae (I-A)-(I-G)), or a pharmaceutical composition described herein. In certain aspects of this embodiment, the T cells in the subject have at least one of enhanced priming, enhanced activation, enhanced migration, enhanced proliferation, enhanced survival, and enhanced cytolytic activity relative to prior to the administration of the compound or pharmaceutical composition. In certain aspects of this embodiment, the T cell activation is characterized by an elevated frequency of y-IFN+ CDS T cells, an elevated frequency of y-IFN+ CD4 T cells, or enhanced levels of IL-2 or granzyme B production by T cells, relative to prior to administration of the compound or pharmaceutical composition. In certain aspects of this embodiment, the number of T cells is elevated relative to prior to administration of the compound or pharmaceutical composition. In certain aspects of this embodiment, the T cell is an antigen-specific CDS T cell. In certain aspects of this embodiment, the T cell is an antigenspecific CD4 T cell. In certain aspects of this embodiment, the antigen presenting cells in the subject have enhanced maturation and activation relative prior to the administration of the compound or pharmaceutical composition. In certain aspects of this embodiment, the antigen presenting cells are dendritic cells. In certain aspects of this embodiment, the maturation of the antigen presenting cells is characterized by increased frequency of CD83+ dendritic cells. In certain aspects of this embodiment, the activation of the antigen presenting cells is characterized by elevated expression of CD80 and CD86 on dendritic cells. In some aspects, compounds of Formulae (I-A)-(I-G), or variations thereof, or a pharmaceutical composition thereof provides general priming of the immune response (i.e., vaccines) to tumors or viruses for boosting/generating anti-viral/tumor immunity.

In another embodiment, the subject matter disclosed herein is directed to a method for treating a cancer, the method comprising administering to a subject in need thereof an effective amount of a compound of Formulae (I-A)-(I-G), or a pharmaceutical composition thereof as further described herein. It is understood that the compound functions by inhibiting Cbl-B in a manner that leads to activated T cells that are able to kill cancer cells, regardless of their origin in the body. In certain aspects of this embodiment, the cancer comprises at least one cancer selected from the group consisting of colorectal cancer, melanoma, non-small cell lung cancer, ovarian cancer, breast cancer, pancreatic cancer, a hematological malignancy, and a renal cell carcinoma. In certain aspects of this embodiment, the cancer has elevated levels of T-cell infiltration. In certain aspects of this embodiment, the cancer cells in the subject selectively have elevated expression of MHC class I antigen expression relative to prior to the administration of the compound or composition.

In the methods described herein, the method can further comprise administering a therapeutic, or chemotherapeutic agent to said subject. For example, such an agent may be an inhibitor of PD-1/PD-1. In certain aspects of this embodiment, the therapeutic or chemotherapeutic agent is administered to the subject simultaneously with the compound or the composition. In certain aspects of this embodiment, the therapeutic or chemotherapeutic agent is administered to the subject prior to administration of the compound or the composition. In certain aspects of this embodiment, the therapeutic or chemotherapeutic agent is administered to the subject after administration of the compound or said composition.

As used herein, “enhancing an immune response” refers to an improvement in any immunogenic response to an antigen. Non-limiting examples of improvements in an immunogenic response to an antigen include enhanced maturation or migration of dendritic cells, enhanced activation of T cells (e.g., CD4 T cells, CDS T cells), enhanced T cell (e.g., CD4 T cell, CDS T cell) proliferation, enhanced B cell proliferation, increased survival of T cells and/or B cells, improved antigen presentation by antigen presenting cells (e.g., dendritic cells), improved antigen clearance, increase in production of cytokines by T cells (e.g., interleukin-2), increased resistance to prostaglandin E2-induced immune suppression, and enhanced priming and/or cytolytic activity of CDS T cells.

In some embodiments, the CDS T cells in the subject have enhanced priming, activation, proliferation and/or cytolytic activity relative to prior to the administration of a compound of Formulae (I-A)-(I-G), or a pharmaceutically acceptable salt, prodrug, metabolite, or derivative thereof. In some embodiments, the CDS T cell priming is characterized by elevated CD44 expression and/or enhanced cytolytic activity in CDS T cells. In some embodiments, the CDS T cell activation is characterized by an elevated frequency of y-IFN+ CDS T cells. In some embodiments, the CDS T cell is an antigen-specific T-cell.

In some embodiments, the CD4 T cells in the subject have enhanced priming, activation, proliferation and/or cytolytic activity relative to prior to the administration of the compound of Formulae (I-A)-(I-G), or a pharmaceutically acceptable salt, prodrug, metabolite, or derivative thereof. In some embodiments, the CD4 T cell priming is characterized by elevated CD44 expression and/or enhanced cytolytic activity in CD4 T cells. In some embodiments, the CD4 T cell activation is characterized by an elevated frequency of y-IFN+ CD4 T cells. In some embodiments, the CD4 T cell is an antigen-specific T-cell.

Accordingly, the presently disclosed compounds of Formulae (I-A)-(I-G), or pharmaceutically acceptable salts, prodrugs, metabolites, or derivatives thereof are useful in treating T cell dysfunctional disorders. A “T cell dysfunctional disorder” is a disorder or condition of T cells characterized by decreased responsiveness to antigenic stimulation.

Thus, the presently disclosed compounds can be used in treating conditions where enhanced immunogenicity is desired, such as increasing tumor immunogenicity for the treatment of cancer.

“Immunogenicity” refers to the ability of a particular substance to provoke an immune response. Tumors are immunogenic and enhancing tumor immunogenicity aids in the clearance of the tumor cells by the immune response. Viruses may also be immunogenic and enhancing/activating immunogenicity may aid in clearance of viral particles by the immune response.

“Tumor immunity” refers to the process in which tumors evade immune recognition and clearance. Thus, as a therapeutic concept, tumor immunity is “treated” when such evasion is attenuated, and the tumors are recognized and attacked by the immune system. Examples of tumor recognition include tumor binding, tumor shrinkage and tumor clearance.

The compounds herein may be used in conjunction with one or more chemotherapeutic agents, in the course of treating a patient. A “chemotherapeutic agent” is a chemical compound or biologic useful in the treatment of cancer. Examples of chemotherapeutic agents include alkylating agents such as thiotepa and cyclophosphamide (CYTOXAN®); alkyl sulfonates such as busulfan, improsulfan, and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; pemetrexed; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CBI-TMI); eleutherobin; pancratistatin; TLK-286; CDP323, an oral alpha-4 integrin inhibitor; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g., calicheamicin, especially calicheamicin gammall and calicheamicin omegall (see, e.g., Nicolaou et al., Angew. Chem Intl. Ed. Engl., 33: 183-186 (1994)); dynemicin, including dynemicin A; an esperamicin; as well as neocarzinostatin chromophore and related chromoprotein enediyne antibiotic chromophores), streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, and floxuridine; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albumin-engineered nanoparticle formulation of paclitaxel (ABRAXANE™), and doxetaxel (TAXOTERE®); chloranbucil; 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluorometlhylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

Additional examples of chemotherapeutic agents that can be deployed in treatment protocols that involve the Cbl-B inhibitor compounds herein, include anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LYII 7018, onapristone, and toremifene (FARESTON®); anti-progesterones; estrogen receptor down-regulators (ERDs); estrogen receptor antagonists such as fulvestrant (FASLODEX®); agents that function to suppress or shut down the ovaries, for example, leutinizing hormone-releasing hormone (LHRH) agonists such as leuprolide acetate (LUPRON® and ELIGARD®), goserelin acetate, buserelin acetate and tripterelin; anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE®), exemestane (AROMASIN®), formestanie, fadrozole, vorozole (RIVISOR®), letrozole (FEMARA®), and anastrozole (ARIMIDEX®). In addition, such definition of chemotherapeutic agents includes bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); as well as troxacitabine (a 1,3-dioxolane nucleoside cytosine analog); anti-sense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); an antiestrogen such as fulvestrant; EGFR inhibitor such as erlotinib or cetuximab; an anti-VEGF inhibitor such as bevacizumab; arinotecan; rmRH (e.g., ABARELIX®); 17AAG (geldanamycin derivative that is a heat shock protein (Hsp) 90 poison), and pharmaceutically acceptable salts, acids or derivatives of any of the above.

Also included in the definition of “chemotherapeutic agent” are: anti-hormonal agents that act to regulate or inhibit hormone action on tumors such as anti-estrogens and selective estrogen receptor modulators (SERMs), including, for example, tamoxifen (including NOL V ADEX®; tamoxifen citrate), raloxifene, droloxifene, 4-hydroxytamoxifen, trioxifene, keoxifene, LYII 7018, onapristone, and FARESTON® (toremifine citrate); aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); antiandrogens such as flutamide, nilutamide, bicalutamide, leuprolide, and goserelin; as well as troxacitabine (a 1,3-dioxolane nucleoside cytosine analog); protein kinase inhibitors; lipid kinase inhibitors; antisense oligonucleotides, particularly those which inhibit expression of genes in signaling pathways implicated in aberrant cell proliferation, such as, for example, PKC-alpha, Ralf and H-Ras; ribozymes such as VEGF expression inhibitors (e.g., ANGIOZYME®) and HER₂ expression inhibitors; vaccines such as gene therapy vaccines, for example, ALLOVECTIN®, LEUVECTIN®, and VAXID®; PROLEUKIN® rIL-2; a topoisomerase 1 inhibitor such as LURTOTECAN®; ABARELIX® rmRH; antiangiogenic agents such as bevacizumab (AV ASTIN®, Genentech); and pharmaceutically acceptable salts, acids and derivatives of any of the above.

In some embodiments, the chemotherapeutic agent is an immunotherapeutic agent. As used herein, an “immunotherapeutic agent” is a compound that enhances the immune system to help fight cancer, specifically or non-specifically. Immunotherapeutics include monoclonal antibodies and non-specific immunotherapies that boost the immune system, such as cytokines, interleukins (e.g., IL-2, IL-7, IL-12, IL-15, IL-21), interferons (e.g., IFN-α, IFN-˜, IFN-γ), GMCSF, thalidomide, (THALOMID®, Celgene), lenalidomide (REVLIMID®, Celgene), pomalidomide (POMALYST®, Celgene), imiquimod (ZYCLARA®, Valeant). Non-limiting examples of monoclonal antibodies that are useful as a chemotherapeutic agent include trastuzumab (HERCEPTIN®, Genentech), bevacizumab (AV ASTIN®, Genentech), cetuximab (ERBITUX®, Bristol-Myers Squibb), panitumumab (VECTIBIX®, Amgen), ipilimumab (YERVOY®, Bristol-Myers Squibb), rituximab (RITUXAN®, Genentech), alemtuzumab (CAMPATH®, Genzyme), ofatumumab (ARZERRA®, Genmab), gemtuzumab ozogamicin (MYLOTARG®, Wyeth), brentuximab vedotin (ADCETRIS®, Seattle Genetics), 90Y-labelled ibritumomab tiuxetan (ZEVALIN®, Biogen Idec), 1311-labelled tositumomab (BEXXAR®, GlaxoSmithKline), ado-trastuzumab emtansine (KADCYLA®, Genentech) blinatumomab (BLINCYTO®, Amgen), pertuzumab (PERJETA®, Genentech), obinutuzumab (GAZYVA®, Genentech), nivolumab (OPDIVO®,) Bristol-Myers Squibb), pembrolizumab (KEYTRUDA®, Merck), pidilizumab (CureTech), Tiragolumab (Roche/Genentech, described in WHO Drug Information (International Nonproprietary Names for Pharmaceutical Substances), Proposed INN: List 117, Vol. 31, No. 2, Jun. 9, 2017 (page 343)), MPDL₃₂₈₀A (Atezolizumab, Roche/Genentech), MDX-1105 (described in WO2007/005874), and MEDI4736 (IMFINZI®, Durvalumab, Medarex). Another useful immunotherapeutic agent is AMP-224 (described in WO2010/027827 and WO2011/066342).

In some embodiments, the compound is administered to the subject at a dose of between about 0.001 μg/kg and about 1,000 mg/kg, including but not limited to about 0.001 μg/kg, about 0.01 μg/kg, about 0.05 μg/kg, about 0.1 μg/kg, about 0.5 μg/kg, about 1 μg/kg, about 10 μg/kg, about 25 μg/kg, about 50 μg/kg, about 100 μg/kg, about 250 μg/kg, about 500 μg/kg, about 1 mg/kg, about 5 mg/kg, about 10 mg/kg, about 25 mg/kg, about 50 mg/kg, about 100 mg/kg, and about 200 mg/kg.

### Exemplary Synthetic Methods

Compounds of Formulae (I-A)-(I-G) can individually be synthesized via one or more of the general synthetic Schemes A-E. It would be understood by a skilled organic chemist that other synthetic pathways could also be devised and that the synthetic methods described herein are neither exclusive nor limiting. Instances of application of one or more of Schemes A-E to specific compounds herein can be found in the Examples.

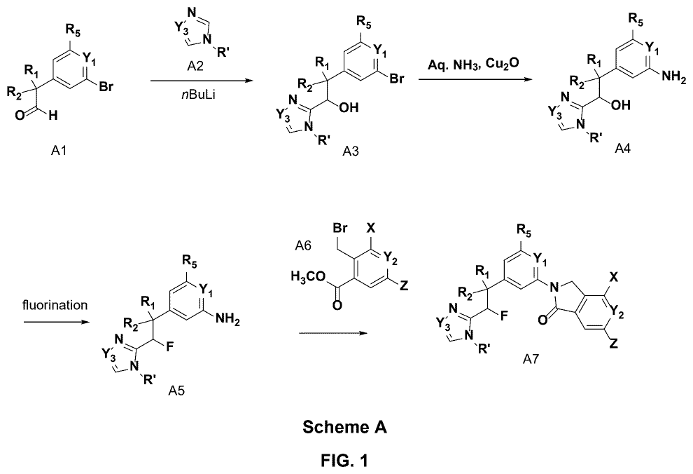

Scheme A (FIG. 1) shows a first method of synthesizing compounds of formulae I-A, I-B, I-C. In Scheme A, Y₁ and Y₂ are as elsewhere described herein; X and Z are as described elsewhere herein; R₁, R₂ and R₅ are as described elsewhere herein. R′ is H or methyl, and Y₃ is N or CH.

Reaction of aldehyde A1 with an aryl lithium, generated when A2 is treated with nBuLi, gives intermediate A3. Subsequently, A3 is converted to aniline A4. This is followed by fluorination reaction of A4, which yields intermediate A5. Reaction of A5 with a benzyl bromide A6 leads to compound A7.

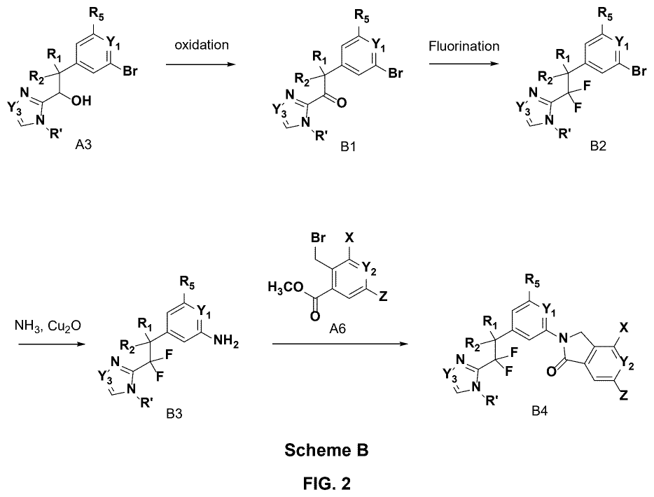

Scheme B, FIG. 2 shows a second method of synthesizing compounds falling within Formulae I-A, I-B, I-C. In Scheme B, Y₁ and Y₂ are as elsewhere described herein; X and Z, are as described elsewhere herein; R₁, R₂, and R₅ are as described elsewhere herein. R′ is H or methyl, and Y₃ is N or CH.

In Scheme B, intermediate A3 can be oxidized to give ketone B1. Treatment of B1 with a fluorinating reagent gives di-fluoro intermediate B2, which is subsequently transformed to aniline B3. Reaction of B3 with a benzyl bromide leads to B4.

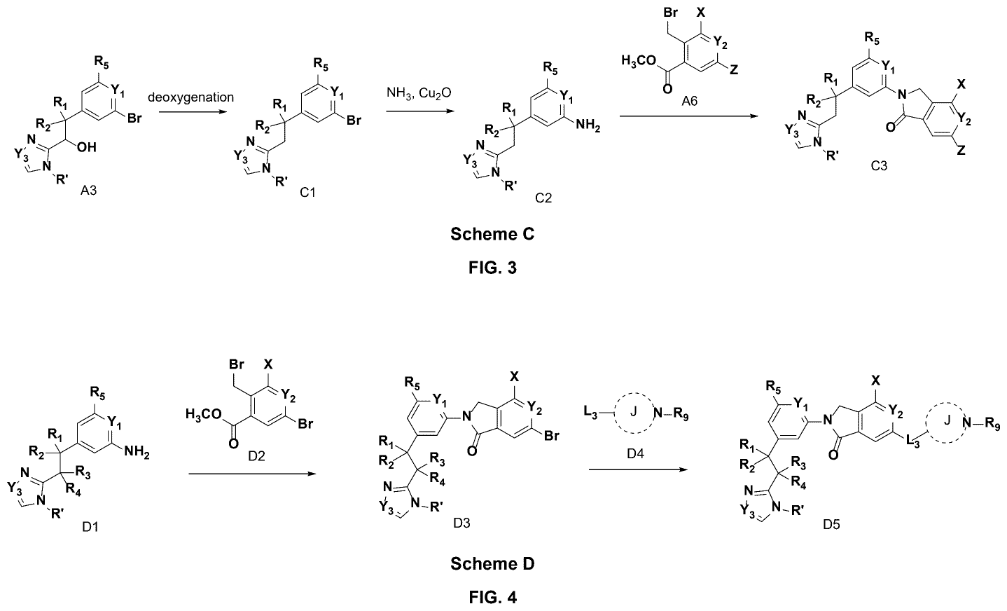

Scheme C, FIG. 3, shows a third method of synthesizing compounds of formulae I-A, I-B, I-C. In Scheme C, Y₁ and Y₂ are as elsewhere described herein; X, Z, are as described elsewhere herein; R₁, R₂ and R₅ are as described elsewhere herein. R′ is H or methyl, and Y₃ is N or CH.

In Scheme C, intermediate A3 can also be deoxygenated to give intermediate C1, which can be transformed to aniline C2. Reaction of intermediate C2 with benzyl Br A6 yields compound C3.

Scheme D, FIG. 4, shows a fourth method of synthesizing compounds of formulae I-A, I-B, I-C, I-D, and I-E. In Scheme D, Y₁ and Y₂ are as elsewhere described herein; X, X₁, Z, Z₁, and Z₂ are as described elsewhere herein; R₁, R₂ and R₅ are as described elsewhere herein. R′ is H or methyl, and Y₃ is N or CH.

In Scheme D, intermediate D1 can react with a benzyl bromide D2, to give intermediate D3. The Br intermediate D3 can subsequently be transformed to compound D5 via a transition metal-catalyzed coupling reaction or a photoredox reaction.

The following examples are offered by way of illustration and not by way of limitation.

### EXAMPLES

Synthesis of various compounds of the invention can be accomplished by utilizing one or more intermediates, as disclosed in Examples 1-15. The manner of use of such intermediates, in conjunction with one or more of Schemes A-D, will be apparent to one of skill in the art seeking to synthesize a compound disclosed herein, or encompassed by Formulae (I-A)-(I-G), even if not explicitly utilized in one of the enumerated Examples herein.

### Example 1: Intermediate A (methyl 5-bromo-2-(bromomethyl)-3-(trifluoromethyl)-benzoate)

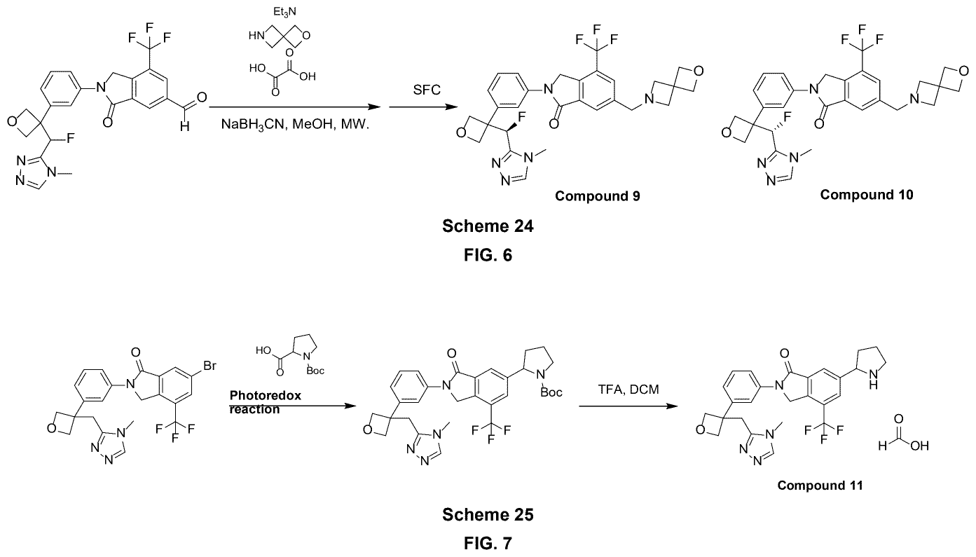

Intermediate A can be synthesized according to Scheme 1, FIG. 6A.

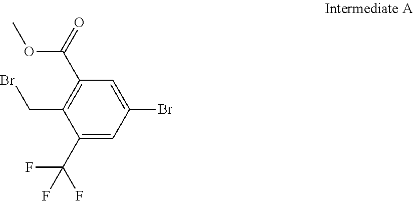 [SMILES: COC(=O)c1cc(Br)cc(C(F)(F)F)c1CBr]

Step A-1 involves synthesis of methyl 2-methyl-3-(trifluoromethyl)benzoate. To a solution of 2-methyl-3-(trifluoromethyl)benzoic acid (10.0 g, 49.0 mmol) in methanol (196 mL) was added sulfuric acid (5.0 mL, 93.1 mmol) and the resulting mixture was heated to 65° C. for 23 h. The reaction was cooled to RT, concentrated, diluted with water (250 mL)/sat. aqueous sodium bicarbonate (250 mL) and extracted with EtOAc (3×200 mL). The organics were combined, dried over sodium sulfate, filtered and evaporated to afford methyl 2-methyl-3-(trifluoromethyl)benzoate (10.3 g, 97% yield). ¹H NMR (400 MHz, CDCl₃) δ 7.91 (d, J=7.8 Hz, 1H), 7.76 (d, J=7.8 Hz, 1H), 7.33 (t, J=7.9 Hz, 1H), 3.92 (s, 3H), 2.64 (q, J=1.4 Hz, 3H).

Step A-2 involves synthesis of methyl 5-bromo-2-methyl-3-(trifluoromethyl)-benzoate. To a solution of methyl 2-methyl-3-(trifluoromethyl)benzoate (10.3 g, 47.3 mmol) in acetic acid (65 mL) were added HNO₃, 70% in water (21.1 mL, 473 mmol) and bromine (2.67 mL, 52.0 mmol) followed by dropwise addition of silver nitrate, 2,5 M in water (24.6 mL, 61.5 mmol) using an addition funnel. The mixture was then stirred at RT for 17 h. The reaction mixture was then poured on ice, diluted with 1 N NaOH (200 mL) and extracted with EtOAc (3×100 mL). The organics were combined, washed with water (3×100 mL), dried over sodium sulfate, filtered and evaporated. The crude mixture was diluted with EtOAc (200 mL) and washed with saturated NaHCO₃ (5×100 mL) and saturated Na₂CO₃ (2×100 mL) to get rid of AcOH. The organic phase was dried over sodium sulfate, filtered and evaporated to afford methyl 5-bromo-2-methyl-3-(trifluoromethyl)benzoate (14.4 g, 100% yield). The product was used without any more purification in next step. ¹H NMR (400 MHz, CDCl₃) δ 8.06 (d, J=2.0 Hz, 1H), 7.88 (d, J=1.9 Hz, 1H), 3.93 (s, 3H), 2.58 (q, J=1.5 Hz, 3H).

Step A-3 involves making methyl 5-bromo-2-(bromomethyl)-3-(trifluoromethyl)-benzoate. A mixture of methyl 5-bromo-2-methyl-3-(trifluoromethyl)benzoate (9.87 g, 33.2 mmol), N-bromosuccinimide (17.7 g, 99.7 mmol) and benzoyl peroxide (3.22 g, 13.3 mmol) in carbon tetrachloride (111 mL) was heated to 75° C. and stirred for 20 h. The mixture was cooled to RT, filtered and concentrated. The residue was purified by chromatography on silica gel (100% heptanes) to afford methyl 5-bromo-2-(bromomethyl)-3-(trifluoromethyl)-benzoate (11.29 g, 90% yield). ¹H NMR (400 MHz, CDCl₃) δ 8.18 (d, J=2.1 Hz, 1H), 7.94 (d, J=2.1 Hz, 1H), 3.99 (s, 3H).

### Example 2: Intermediate B (6-(5-Azaspiro[2.4]heptan-5-ylmethyl)-2-cyclopropyl-pyrimidine-4-carboxylic acid)

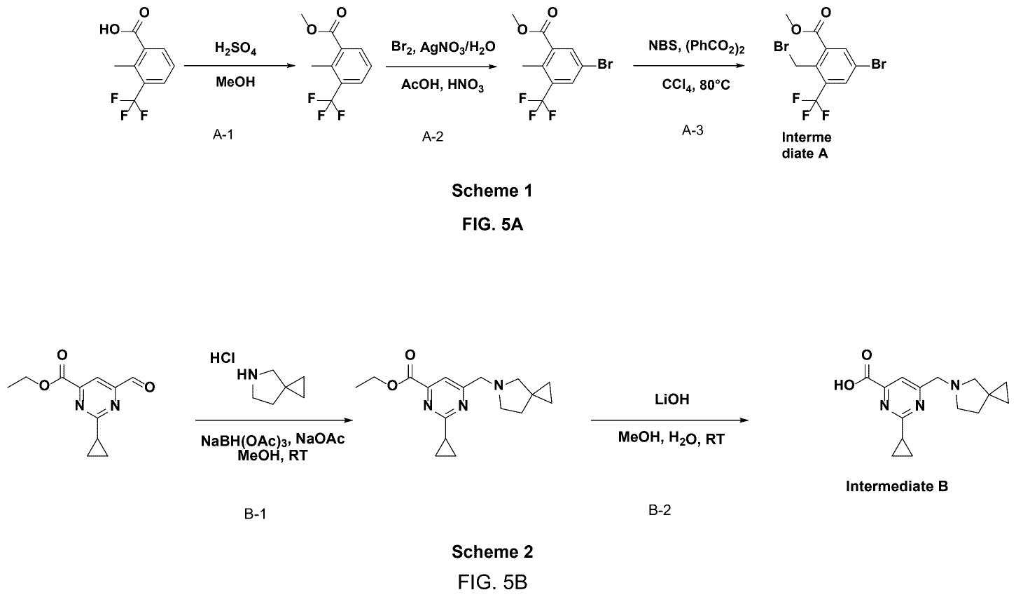

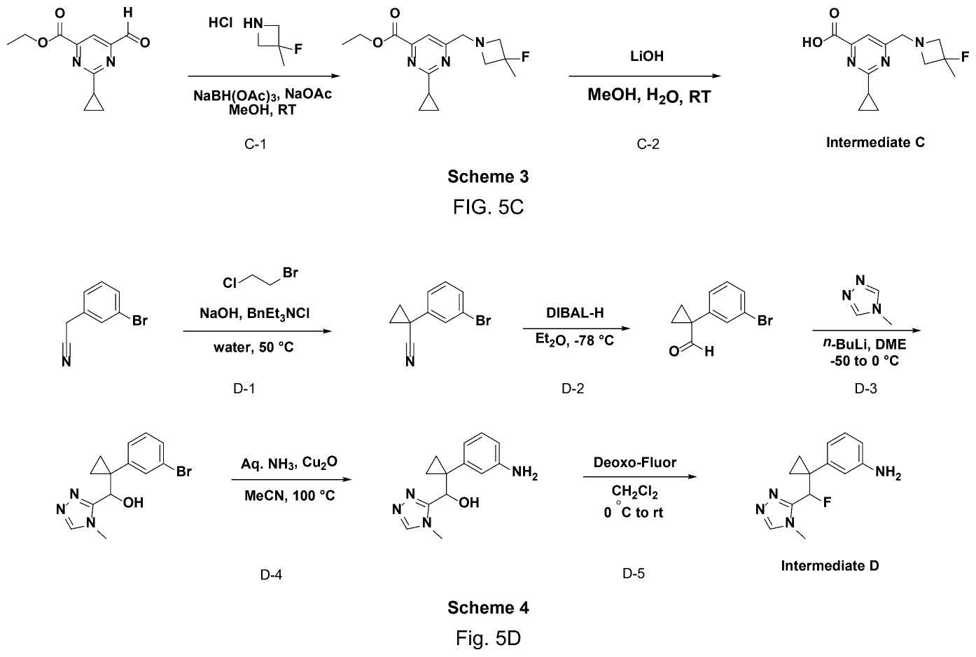

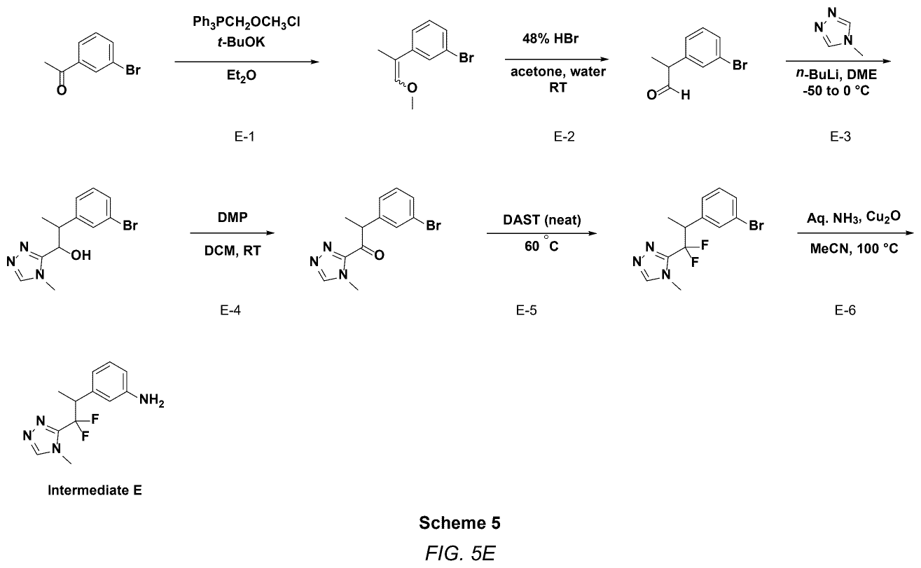

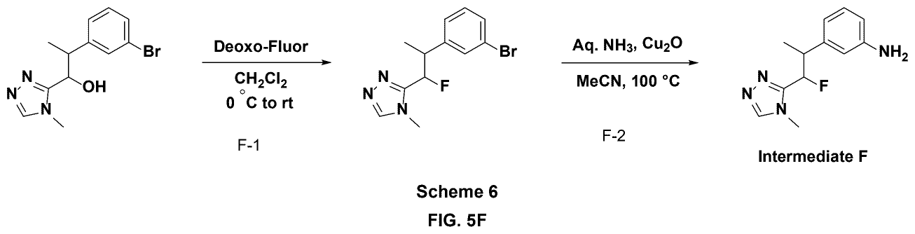

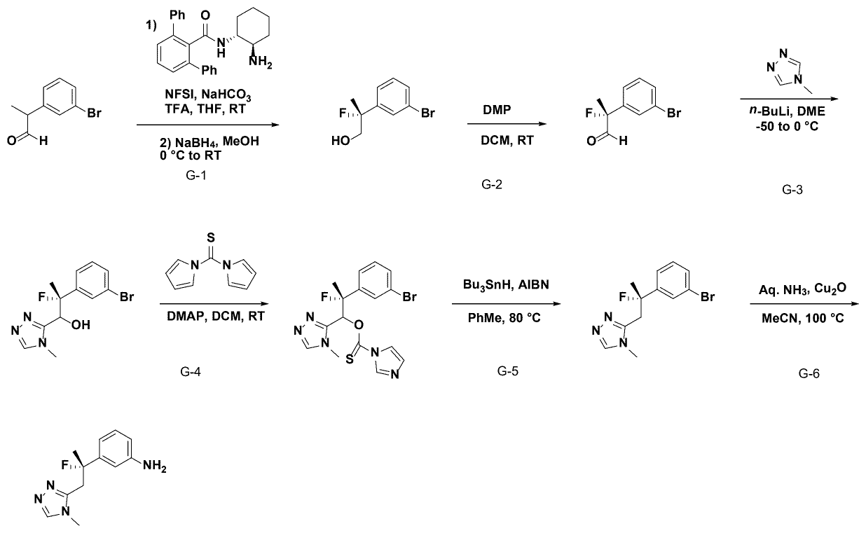

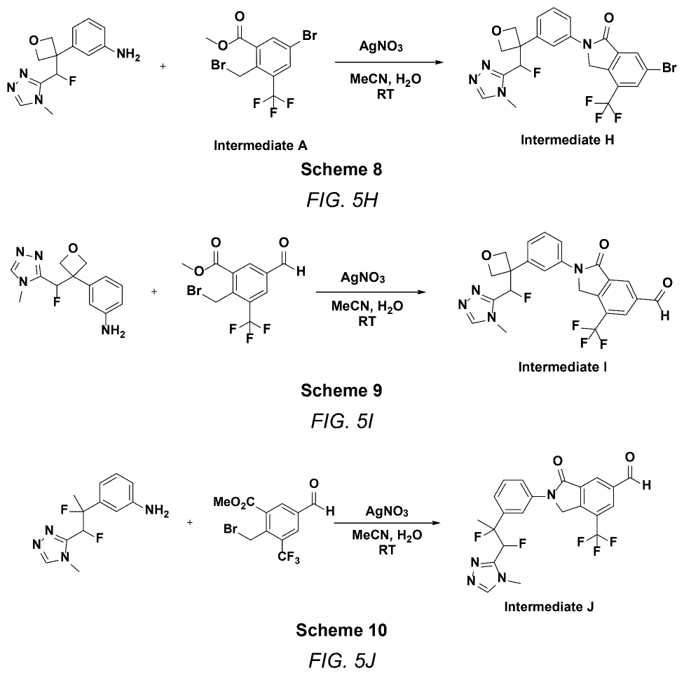

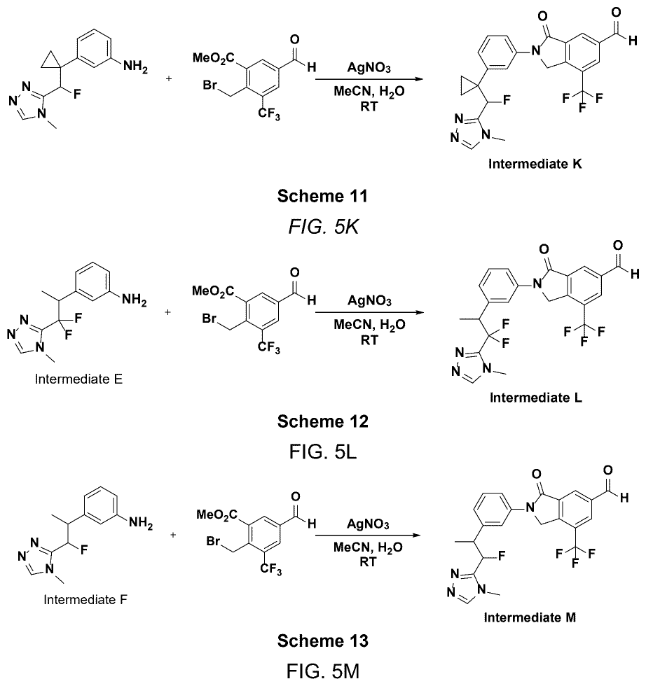

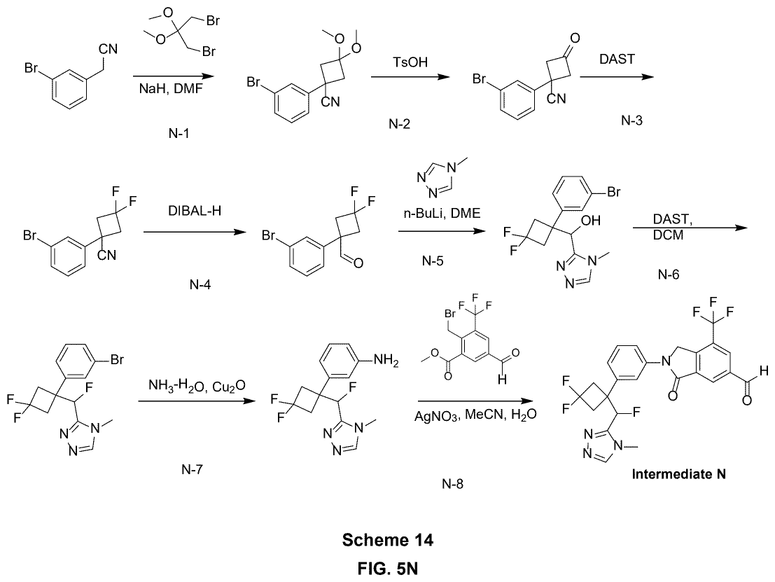

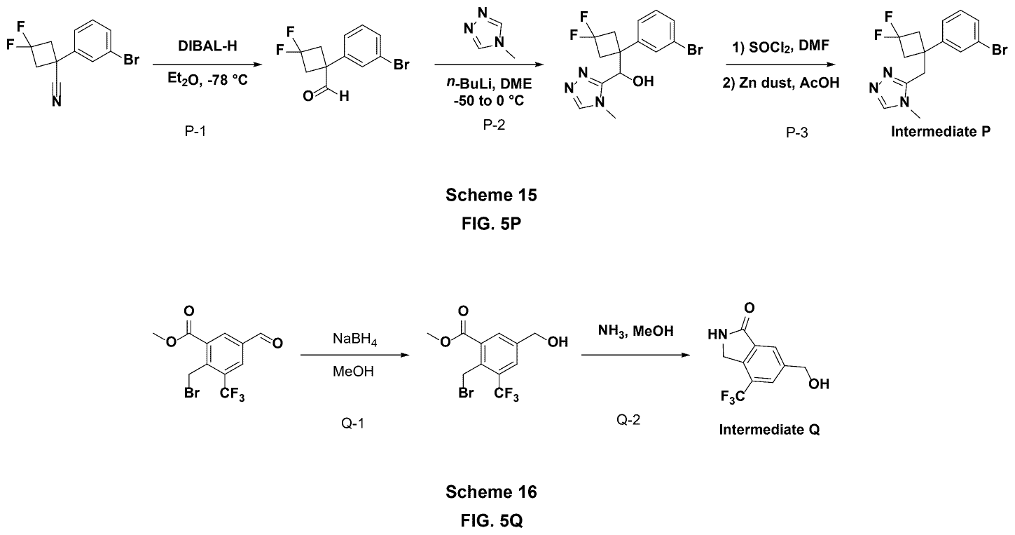

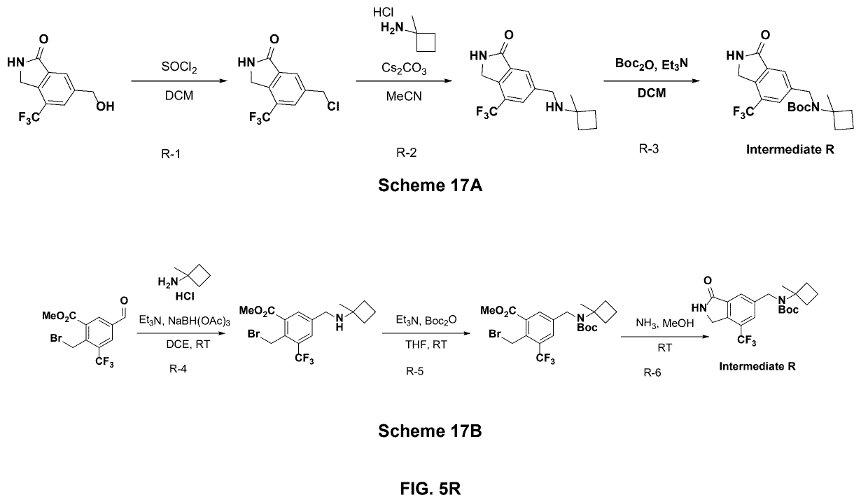

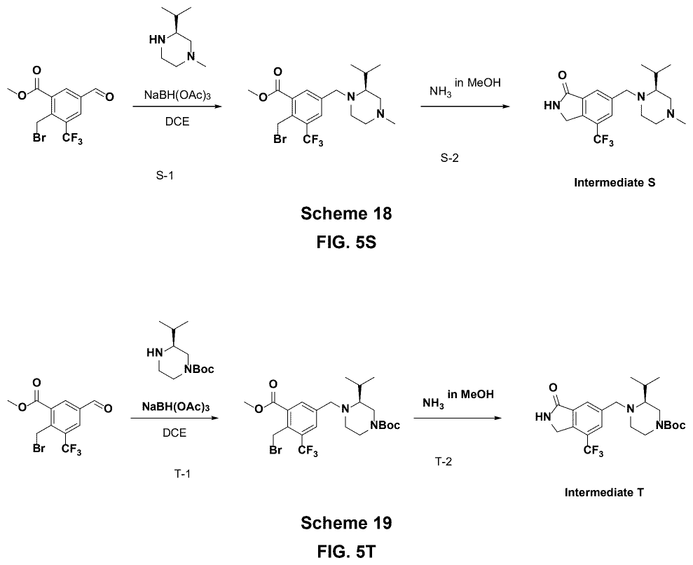

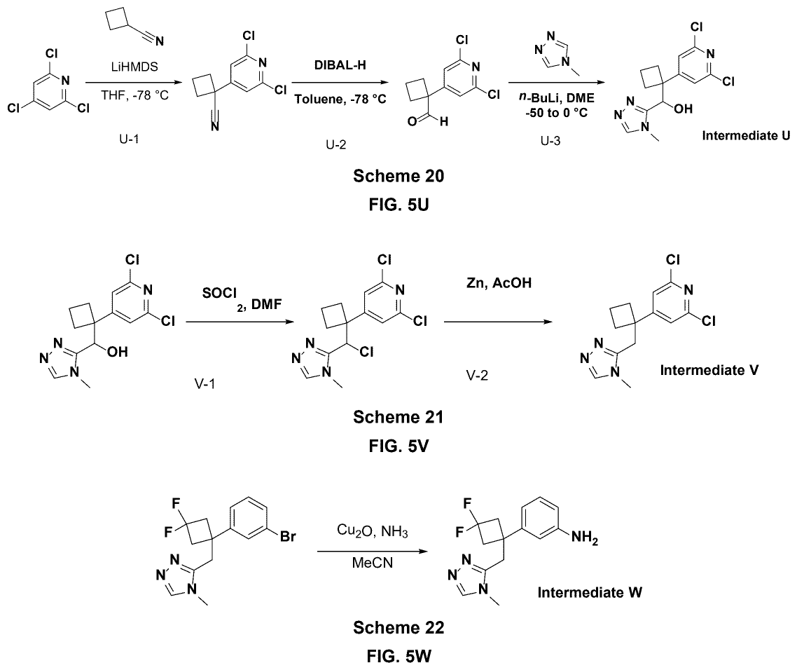

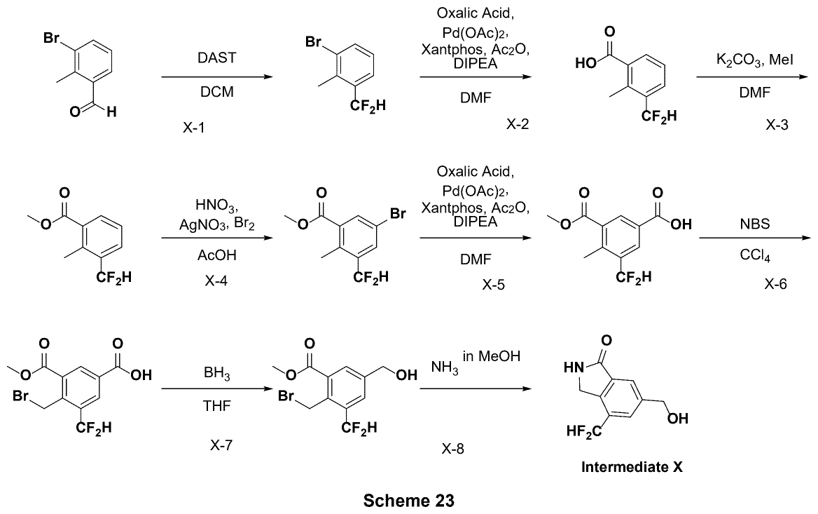

Intermediate B can be synthesized according to Scheme 2, FIG. 5B.

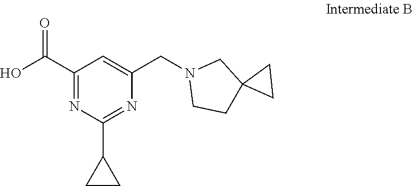 [SMILES: O=C(O)c1cc(CN2CCC3(CC3)C2)nc(C2CC2)n1]

In step B-1, ethyl 6-(5-azaspiro[2.4]heptan-5-ylmethyl)-2-cyclopropylpyrimidine-4-carboxylate is made as follows. To ethyl 2-cyclopropyl-6-formylpyrimidine-4-carboxylate (300 mg, 1.36 mmol) in methanol (6.8 mL) were added 5-azaspiro[2.4]heptane hydrochloride (310 mg, 2.32 mmol) and sodium acetate (340 mg, 4.09 mmol). The reaction mixture was stirred at RT for 15 min and then sodium triacetoxyborohydride (858 mg, 4.09 mmol) was added. The reaction was stirred at RT for 16 h. The reaction was diluted with sat. aqueous sodium bicarbonate (25 mL) and extracted with DCM (3×25 mL). The organic phases were combined, dried over sodium sulfate, filtered and concentrated under reduced pressure to afford ethyl 6-(5-azaspiro[2.4]heptan-5-ylmethyl)-2-cyclopropyl-pyrimidine-4-carboxylate which was used without further purification.

In step B-2, 6-(5-azaspiro[2.4]heptan-5-ylmethyl)-2-cyclopropylpyrimidine-4-carboxylic acid is made as follows. To ethyl 6-(5-azaspiro[2.4]heptan-5-ylmethyl)-2-cyclopropylpyrimidine-4-carboxylate previously obtained in methanol (10 mL) was added 1 M lithium hydroxide (10 mL, 10.0 mmol). The reaction mixture was stirred at RT for 50 min. The reaction was diluted with 1 M HCl until pH 7 was reached and the mixture was concentrated to about 2 mL total volume. The residue was purified by chromatography on C18 silica gel (0-60% acetonitrile in ammonium bicarbonate, pH=10) to afford 6-(5-azaspiro[2.4]heptan-5-ylmethyl)-2-cyclopropylpyrimidine-4-carboxylic acid (246 mg, 66%) as a white solid. LCMS (ESI) m/z: 274.2 [M+H]+. 1H NMR (400 MHz, CD₃OD) 7.66 (s, 1H), 3.75 (s, 2H), 2.85 (t, J=6.9 Hz, 2H), 2.61 (s, 2H), 2.37-2.18 (m, 1H), 1.85 (t, J=6.9 Hz, 2H), 1.27-1.13 (m, 2H), 1.12-0.96 (m, 2H), 0.65-0.45 (m, 4H).

### Example 3: Intermediate C (2-Cyclopropyl-6-((3-fluoro-3-methylazetidin-1-yl)methyl)pyrimidine-4-carboxylic acid)

Intermediate C can be synthesized according to Scheme 3, FIG. 5C.

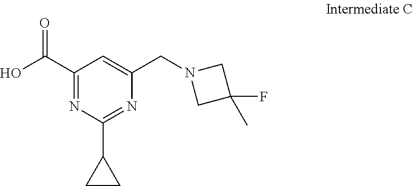 [SMILES: CC1(F)CN(Cc2cc(C(=O)O)nc(C3CC3)n2)C1]

In step C-1, ethyl 2-cyclopropyl-6-((3-fluoro-3-methylazetidin-1-yl)methyl)pyrimidine-4-carboxylate is made as follows. To ethyl 2-cyclopropyl-6-formylpyrimidine-4-carboxylate (300 mg, 1.36 mmol) in methanol (6.8 mL) were added 3-fluoro-3-methyl-azetidine hydrochloride (288 mg, 2.32 mmol) and triethylamine (0.32 mL, 2.32 mmol). The reaction mixture was stirred at 100° C. for 1 min in the microwave and was cooled to RT. Sodium cyanoborohydride (171 mg, 2.72 mmol) was added and the reaction was stirred at 100° C. for 2 h in the microwave. The reaction mixture was taken directly to the next step.

In step C-2, 2-cyclopropyl-6-((3-fluoro-3-methylazetidin-1-yl)methyl)pyrimidine-4-carboxylic acid is made as follows. The mixture containing ethyl 2-cyclopropyl-6-((3-fluoro-3-methylazetidin-1-yl)methyl)pyrimidine-4-carboxylate previously obtained was diluted with methanol (4 mL) and 1 M lithium hydroxide (12 mL, 12.0 mmol) was added. The reaction mixture was stirred at RT for 50 min. The reaction was diluted with 1 M HCl until pH 7 was concentrated to about 2 mL total volume. The residue was purified by chromatography on C18 silica gel (0-70% acetonitrile in ammonium bicarbonate, pH=10) to afford 2-cyclopropyl-6-((3-fluoro-3-methylazetidin-1-yl)methyl)pyrimidine-4-carboxylic acid (90 mg, 25%) as a yellow solid. LCMS (ESI) m/z: 266.1 [M+H]+. ¹H NMR (400 MHz, CD₃OD) 7.57 (s, 1H), 3.79 (s, 2H), 3.59-3.37 (m, 4H), 2.33-2.17 (m, 1H), 1.62 (t, J=26.3 Hz, 3H), 1.23-1.13 (m, 2H), 1.08-1.00 (m, 2H).

### Example 4: Intermediate D (3-(1-(fluoro(4-methyl-4H-1,2,4-triazol-3-yl)methyl)cyclopropyl)aniline)

Intermediate D can be synthesized according to Scheme 4, FIG. 5D.

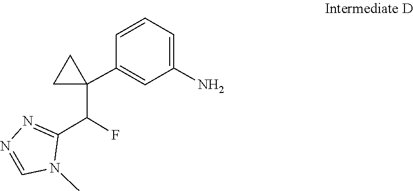 [SMILES: Cn1cnnc1C(F)C1(c2cccc(N)c2)CC1]

In step D-1, 1-(3-bromophenyl)cyclopropanecarbonitrile is made as follows. A mixture of 2-(3-bromophenyl)acetonitrile (6.00 g, 30.6 mmol), 1-bromo-2-chloroethane (3.82 mL, 45.9 mmol) and benzyltriethylammonium chloride (139 mg, 0.61 mmol) in 6 N aqueous sodium hydroxide (38.3 mL, 223 mmol) was stirred for 18 h at 50° C.. The reaction mixture was diluted with EtOAc (200 mL) and water (200 mL). The layers were separated, the aqueous phase was extracted with EtOAc (200 mL), the combined organic phases were washed with 1 N HCl (100 mL), washed with 1 N aqueous potassium carbonate (100 mL), washed with brine (100 mL), dried over magnesium sulfate, filtered and concentrated under reduced pressure to afford 1-(3-bromophenyl)cyclopropanecarbonitrile (6.57 g, 97% yield) as a dark brown solid. ¹H NMR (400 MHz, CDCl₃) 7.44-7.40 (m, 2H), 7.27-7.20 (m, 2H), 1.77-1.73 (m, 2H), 1.43-1.39 (m, 2H).

In step D-2, 1-(3-bromophenyl)cyclopropanecarbaldehyde is made as follows. DIBAL-H, 1.0 M in hexanes (51.6 mL, 51.6 mmol) was added slowly to a solution of 1-(3-bromophenyl)cyclopropanecarbonitrile (7.64 g, 34.4 mmol) in diethyl ether (115 mL) at −78° C. The resulting mixture was stirred for 2 h at the same temperature. The reaction was carefully quenched with 50 mL of 10% aqueous HCl and allowed to warm to RT. The reaction mixture was diluted with EtOAc (200 mL) and water (200 mL). The layers were separated, the aqueous phase was extracted with EtOAc (200 mL), the combined organic phases were washed with saturated sodium bicarbonate (100 mL), washed with water (100 mL), washed with brine (100 mL), dried over magnesium sulfate, filtered and concentrated under reduced pressure to afford 1-(3-bromophenyl)cyclopropanecarbaldehyde (7.62 g, 98% yield) as an orange oil. ¹H NMR (400 MHz, DMSO-d6) 13.48 (s, 1H), 9.91 (s, 1H), 8.77-8.75 (m, 1H), 8.09-8.05 (m, 2H), 7.63 (t, J=8.0 Hz, 1H), 6.54 (s, 1H).

In step D-3, (1-(3-bromophenyl)cyclopropyl)(4-methyl-4H-1,2,4-triazol-3-yl)methanol is made as follows. A solution of n-butyllithium, 2.64 M in hexanes (12.7 mL, 33.6 mmol) was added drop wise to a solution of 4-methyl-1,2,4-triazole (2.79 g, 33.6 mmol) in anhydrous DME (600 mL) at −50° C. The resulting mixture was stirred at −50° C. for 1 h before a solution of 1-(3-bromophenyl)cyclopropanecarbaldehyde (6.88 g, 30.6 mmol) in DME (25 mL) was added dropwise. The reaction was gradually allowed to warm to 0° C. over 1 h. The reaction was quenched with water (100 mL) and DME was evaporated under reduced pressure. The residue was diluted with 4:1 CHCl₃/IPA (200 mL). The layers were separated, the aqueous phase was extracted with 4:1 CHCl₃/IPA (4×200 mL), the combined organic phases were dried over magnesium sulfate, filtered and concentrated under reduced pressure. The residue was purified by chromatography on silica gel (2-12% MeOH in DCM) to afford (1-(3-bromophenyl)cyclopropyl)(4-methyl-4H-1,2,4-triazol-3-yl)methanol (4.82 g, 51% yield) as a white solid. LCMS (ESI) m/z: 308.2/310.2 [M+H]⁺ (Br pattern). ¹H NMR (400 MHz, CDCl₃) 7.81 (s, 1H), 7.37-7.33 (m, 2H), 7.11-7.04 (m, 2H), 5.83 (s, 1H), 4.70 (s, 1H), 3.07 (s, 3H), 1.22-1.16 (m, 1H), 1.15-1.10 (m, 1H), 0.93-0.87 (m, 1H), 0.84-0.78 (m, 1H).

In step D-4, (1-(3-Aminophenyl)cyclopropyl)(4-methyl-4H-1,2,4-triazol-3-yl)methanol is made as follows. Copper(I) oxide (278 mg, 1.95 mmol) was added to a mixture of (1-(3-bromophenyl)cyclopropyl)(4-methyl-4H-1,2,4-triazol-3-yl)methanol (1.00 g, 3.25 mmol) and conc. aqueous ammonia (6.0 mL) in acetonitrile (6.5 mL) under nitrogen in a microwave vial. The vial was sealed and the reaction was stirred for 18 h at 100° C. The reaction was diluted with 4:1 CHCl₃/IPA (50 mL), water (25 mL) and conc. aqueous ammonia (25 mL). The layers were separated, the aqueous phase was extracted with 4:1 CHCl₃/IPA (10×50 mL), the combined organic phases were dried over magnesium sulfate, filtered and concentrated under reduced pressure. The residue was purified by chromatography on C18 silica gel (0-50% acetonitrile in ammonium bicarbonate, pH=10) to afford (1-(3-aminophenyl)cyclopropyl)(4-methyl-4H-1,2,4-triazol-3-yl)methanol (472 mg, 60% yield) as a white solid. LCMS (ESI) m/z: 245.2 [M+H]⁺.

In step D-5, 3-(1-(fluoro(4-methyl-4H-1,2,4-triazol-3-yl)methyl)cyclopropyl)aniline is made as follows. To a solution of (1-(3-aminophenyl)cyclopropyl)(4-methyl-4H-1,2,4-triazol-3-yl)methanol (652 mg, 2.67 mmol) in DCM (27 mL) was added Deoxo-Fluor (50% w/w in toluene) (3.91 mL, 8.84 mmol) dropwise at 0° C. (monitoring internal temperature <5° C.). The reaction was allowed to warm to RT and stirred for 1 h. The reaction was cooled to 0° C. and was quenched with slow addition of water (25 mL). The reaction mixture was extracted with 4:1 CHCl₃/IPA (6×50 mL) and the combined organic layers were dried over sodium sulfate. The residue was purified by chromatography on C18 silica gel (0-50% acetonitrile in ammonium formate, pH=4) to afford 3-(1-(fluoro(4-methyl-4H-1,2,4-triazol-3-yl)methyl)cyclopropyl)aniline (393 mg, 60% yield) as a yellow solid. LCMS (ESI) m/z: 247.3 [M+H]⁺.

### Example 5: Intermediate E (3-(1,1-Difluoro-1-(4-methyl-4H-1,2,4-triazol-3-yl)propan-2-yl)aniline)

Intermediate E can be synthesized according to Scheme 5, FIG. 5E.

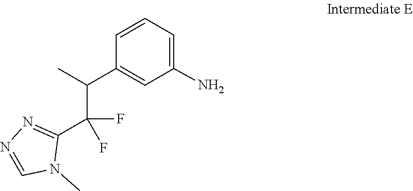 [SMILES: CC(c1cccc(N)c1)C(F)(F)c1nncn1C]

In step E-1, 1-bromo-3-(1-methoxyprop-1-en-2-yl)benzene is made as follows. To a stirred suspension of methoxymethyl triphenylphosphonium chloride (111 g, 324 mmol) in diethyl ether (1.1 L) at 0° C. was added potassium tert-butoxide (38.8 g, 346 mmol) in portions. After 30 min, a solution of 3′-bromoacetophenone (28.6 mL, 216 mmol) in diethyl ether (150 mL) was added dropwise to the reaction mixture, which was then allowed to warm up to RT and stirred for 17 h. The reaction mixture was concentrated under reduced pressure to around 600 mL and washed with sat. aqueous ammonium chloride (200 mL). The organic layer was separated and concentrated under reduced pressure to give and solid suspension. This solid suspension was diluted with heptanes (300 mL) and stirred for 30 min. The precipitate was filtered through sand and washed with heptanes. The filtrate was concentrated under reduced pressure and passed through a silica gel pad eluting with 5% ethyl acetate in heptanes to afford 1-bromo-3-(1-methoxyprop-1-en-2-yl)benzene (47.0 g, 96% yield).

In step E-2, 2-(3-bromophenyl)propanal is made as follows. HBr, 48% in water (25.8 mL, 228 mmol) was added dropwise to a solution of 1-bromo-3-(1-methoxyprop-1-en-2-yl)benzene (47.0 g, 207 mmol) in acetone (200 mL) and water (51 mL) cooled to 0° C. The reaction was then allowed to warm to RT and stirred for 3 days. The reaction was then quenched with sat. aqueous sodium bicarbonate and acetone was evaporated. The resulting aqueous mixture was extracted with DCM (3×200 mL). The organic phases were combined, dried over sodium sulfate, filtered and concentrated under reduced pressure. The residue was purified by chromatography on silica gel (0-30% DCM in heptanes) to afford 2-(3-bromophenyl)propanal (32.0 g, 73% yield). ¹H NMR (400 MHz, CDCl₃) 9.67 (d, J=1.4 Hz, 1H), 7.44 (ddd, J=8.0, 1.9, 1.0 Hz, 1H), 7.37 (t, J=1.8 Hz, 1H), 7.25 (t, J=8.0 Hz, 1H), 7.16-7.12 (m, 1H), 3.61 (qd, J=7.1, 1.1 Hz, 1H), 1.45 (t, J=8.0 Hz, 1H).

In step E-3, 2-(3-bromophenyl)-1-(4-methyl-4H-1,2,4-triazol-3-yl)propan-1-ol is made as follows. A solution of n-butyllithium, 2.5 M in hexanes (16.5 mL, 41.3 mmol) was added drop wise to a solution of 4-methyl-1,2,4-triazole (3.43 g, 41.3 mmol) in anhydrous DME (375 mL) at −50° C. The resulting mixture was stirred at −50° C. for 1 h before a solution of 2-(3-bromophenyl)propanal (8.0 g, 37.6 mmol) in DME (30 mL) was added dropwise. The reaction was gradually allowed to warm to 0° C. over 1 h. The reaction was quenched with water (100 mL) and DME was evaporated under reduced pressure. The residue was diluted with 4:1 CHCl₃/IPA (200 mL). The layers were separated, the aqueous phase was extracted with 4:1 CHCl₃/IPA (4×200 mL), the combined organic phases were dried over magnesium sulfate, filtered and concentrated under reduced pressure. The residue was purified by chromatography on silica gel (0-15% MeOH in DCM) to afford 2-(3-bromophenyl)-1-(4-methyl-4H-1,2,4-triazol-3-yl)propan-1-ol (6.20 g, 56% yield). LCMS (ESI) m/z: 296.1/298.2 [M+H]⁺ (Br pattern).

In step E-4, 2-(3-bromophenyl)-1-(4-methyl-4H-1,2,4-triazol-3-yl)propan-1-one is made as follows. DMP (17.8 g, 42 mmol) was added in one portion to a solution of 2-(3-bromophenyl)-1-(4-methyl-4H-1,2,4-triazol-3-yl)propan-1-ol (6.22 g, 21.0 mmol) in DCM (100 mL) at RT. The resulting mixture was stirred for 20 h at the same temperature. The reaction was quenched with water (100 mL) and diluted 4:1 CHCl₃/IPA (100 mL). The layers were separated, the aqueous phase was extracted with 4:1 CHCl₃/IPA (3×100 mL), the combined organic phases were dried over sodium sulfate, filtered and concentrated under reduced pressure. The residue was purified by chromatography on silica gel (0-100% EtOAc in DCM) to afford 2-(3-bromophenyl)-1-(4-methyl-4H-1,2,4-triazol-3-yl)propan-1-one (5.20 g, 84% yield). LCMS (ESI) m/z: 294.2/296.1 [M+H]⁺ (Br pattern).

In step E-5, 3-(2-(3-bromophenyl)-1,1-difluoropropyl)-4-methyl-4H-1,2,4-triazole is made as follows. A mixture of DAST (25 mL, 189 mmol) and 2-(3-bromophenyl)-1-(4-methyl-4H-1,2,4-triazol-3-yl)propan-1-one (2.70 g, 9.18 mmol) as stirred at 60° C. for 60 h. The reaction was cooled to 0° C., carefully quenched with sat. aqueous sodium bicarbonate until pH 8 was reached and diluted with DCM (100 mL). The layers were separated, the aqueous phase was extracted with DCM (2×100 mL), the combined organic phases were washed with brine (100 mL), dried over sodium sulfate, filtered and concentrated under reduced pressure. The residue was purified by chromatography on silica gel (0-100% EtOAc in DCM) to afford 3-(2-(3-bromophenyl)-1,1-difluoropropyl)-4-methyl-4H-1,2,4-triazole (900 mg, 31% yield). LCMS (ESI) m/z: 316.1/318.12 [M+H]⁺ (Br pattern).

In step E-6, 3-(1,1-Difluoro-1-(4-methyl-4H-1,2,4-triazol-3-yl)propan-2-yl)aniline is made as follows. Copper(I) oxide (462 mg, 3.23 mmol) was added to a mixture of 3-(2-(3-bromophenyl)-1,1-difluoropropyl)-4-methyl-4H-1,2,4-triazole (1.70 g, 5.38 mmol) and conc. aqueous ammonia (35 mL) in acetonitrile (15 mL) under nitrogen in a sealed tube. The tube was sealed and the reaction was stirred for 18 h at 100° C. The reaction was diluted with 4:1 CHCl₃/IPA (100 mL), water (50 mL) and conc. aqueous ammonia (50 mL). The layers were separated, the aqueous phase was extracted with 4:1 CHCl₃/IPA (4×100 mL), the combined organic phases were dried over magnesium sulfate, filtered and concentrated under reduced pressure to afford 3-(1,1-difluoro-1-(4-methyl-4H-1,2,4-triazol-3-yl)propan-2-yl)aniline (1.20 g, 88% yield). LCMS (ESI) m/z: 253.3 [M+H]⁺.

### Example 6: Intermediate F (3-(1-fluoro-1-(4-methyl-4H-1,2,4-triazol-3-yl)propan-2-yl)aniline)

Intermediate F can be synthesized according to Scheme 6, FIG. 5F.

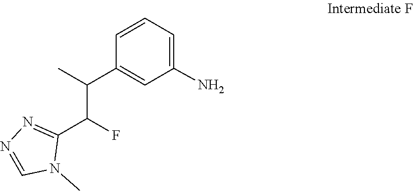 [SMILES: CC(c1cccc(N)c1)C(F)c1nncn1C]

In step F-1, 3-(2-(3-bromophenyl)-1-fluoropropyl)-4-methyl-4H-1,2,4-triazole is made as follows. To a solution of 2-(3-bromophenyl)-1-(4-methyl-4H-1,2,4-triazol-3-yl)propan-1-ol (3.13 g, 10.6 mmol) (previously prepared in synthesis of Intermediate E) in DCM (151 mL) was added Deoxo-Fluor (50% w/w in toluene) (10.2 mL, 23.3 mmol) dropwise at 0° C. (monitoring internal temperature <5° C.). The reaction was allowed to warm to RT and stirred for 1 h. The reaction was cooled to 0° C. and was quenched with slow addition of water (100 mL). The reaction mixture was extracted with DCM (3×100 mL) and the combined organic layers were dried over sodium sulfate. The residue was purified by chromatography on silica gel (0-5% MeOH in DCM) to afford 3-(2-(3-bromophenyl)-1-fluoropropyl)-4-methyl-4H-1,2,4-triazole (2.09 g, 66% yield). LCMS (ESI) m/z: 298.2/300.1 [M+H]⁺ (Br pattern).

In step F-2, 3-(1-fluoro-1-(4-methyl-4H-1,2,4-triazol-3-yl)propan-2-yl)aniline, is made as follows. Copper(I) oxide (602 mg, 4.21 mmol) was added to a mixture of 3-(2-(3-bromophenyl)-1-fluoropropyl)-4-methyl-4H-1,2,4-triazole (2.09 g, 7.00 mmol) and conc. aqueous ammonia (35 mL) in acetonitrile (14 mL) under nitrogen in a sealed tube. The tube was sealed and the reaction was stirred for 18 h at 100° C. The reaction was diluted with 4:1 CHCl₃/IPA (100 mL), water (50 mL) and conc. aqueous ammonia (50 mL). The layers were separated, the aqueous phase was extracted with 4:1 CHCl₃/IPA (5×100 mL), the combined organic phases were dried over sodium sulfate, filtered and concentrated under reduced pressure to afford 3-(1-fluoro-1-(4-methyl-4H-1,2,4-triazol-3-yl)propan-2-yl)aniline (1.82 g, 100% yield). LCMS (ESI) m/z: 235.3 [M+H]⁺.

### Example 7: Intermediate G ((S)-3-(2-fluoro-1-(4-methyl-4H-1,2,4-triazol-3-yl)propan-2-yl)aniline)

Intermediate G can be synthesized according to Scheme 7, FIG. 5G.

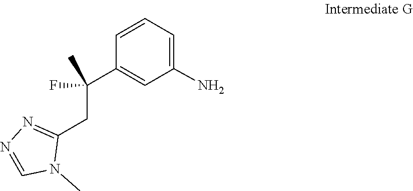 [SMILES: Cn1cnnc1C[C@](C)(F)c1cccc(N)c1]

In step G-1, (R)-2-(3-bromophenyl)-2-fluoropropan-1-ol is made as follows. Sodium bicarbonate (840 mg, 10.0 mmol) and N-fluorobenzenesulfonimide (3.15 g, 10.0 mmol) were added sequentially to a mixture of 2-(3-bromophenyl)propanal (2.13 g, 10.0 mmol), N-[(1R,2R)-2-aminocyclohexyl]-2,6-diphenyl-benzamide (741 mg, 2.00 mmol) and trifluoroacetic acid (154 uL, 2.00 mmol) in THF (30 mL) at RT. The resulting mixture was stirred at RT for 4 h, diluted with MeOH (100 mL) and cooled to O ° C. Sodium borohydride (3.78 g, 100 mmol) was added in 3 portions over the course of 10 min, the reaction was allowed to warm to RT and stirred for 90 min. The reaction was quenched with sat. aqueous ammonium chloride (50 mL), diluted with ethyl acetate (250 mL) and the resulting mixture was stirred for 30 min. The layers were separated and the aqueous phase was extracted with EtOAc (4×100 mL). The combined organic layers were dried over sodium sulfate, filtered, and concentrated under reduced pressure. The residue was purified by chromatography on silica gel (0-60% EtOAc in heptanes) to afford (R)-2-(3-bromophenyl)-2-fluoropropan-1-ol (1.89 g, 81% yield) as a colorless oil. ¹H NMR (400 MHz, CDCl₃) 7.58-7.52 (m, 1H), 7.48-7.42 (m, 1H), 7.31-7.23 (m, 2H), 3.86-3.68 (m, 2H), 1.67 (d, J=22.6 Hz, 3H). % ee was determined to be 80% (Analytical Column: ChiralPak IA, 250 mm×4.6 mm ID, 5 μm; Mobile Phase: 5:95 EtOH:Hexane (0.1% DEA); Isocratic Flow: 1 mL/min, (pressure was 54.4 bars); Column Temp.: ˜26° C.; Run Time: 18 min.; Wavelength: 220 nm; RT peak #1=RT1=8.3 min. (width at mid height=W1=0.1469 min.); RT peak #2=RT_{2=14.6} min. (width at mid height=W2=0.2974 min.)).

In step G-2, (R)-2-(3-bromophenyl)-2-fluoropropanal is made as follows. DMP (3.78 g, 8.92 mmol) was added in one portion to a solution of (R)-2-(3-bromophenyl)-2-fluoropropan-1-ol (1.89 g, 8.11 mmol) in DCM (25 mL) at RT. The resulting mixture was stirred for 1 h at the same temperature. The reaction was filtered through a pad of silica gel (eluting with 100 mL of DCM) and the filtrate was concentrated under reduced pressure to afford (R)-2-(3-bromophenyl)-2-fluoropropanal (1.72 g, 92% yield). ¹H NMR (400 MHz, CDCl₃) 9.70 (d, J=4.7 Hz, 1H), 7.58 (t, J=1.8 Hz, 1H), 7.50 (dt, J=7.3, 1.5 Hz, 1H), 7.31 (ddd, J=14.2, 9.6, 4.8 Hz, 2H), 1.77 (d, J=22.7 Hz, 3H).

In step G-3, (2R)-2-(3-bromophenyl)-2-fluoro-1-(4-methyl-4H-1,2,4-triazol-3-yl)propan-1-ol is made as follows. A solution of n-butyl-lithium, 2.5 M in hexanes (1.90 mL, 4.76 mmol) was added drop wise to a solution of 4-methyl-1,2,4-triazole (396 mg, 4.76 mmol) in anhydrous DME (60 mL) at −50° C. The resulting mixture was stirred at −50° C. for 1 h before a solution of (R)-2-(3-bromophenyl)-2-fluoropropanal (1.00 g, 4.33 mmol) in DME (5 mL) was added dropwise. The reaction was gradually allowed to warm to 0° C. over 1 h. The reaction was quenched with water (25 mL) and DME was evaporated under reduced pressure. The residue was diluted with 4:1 CHCl₃/IPA (50 mL). The layers were separated, the aqueous phase was extracted with 4:1 CHCl₃/IPA (4×50 mL), the combined organic phases were dried over magnesium sulfate, filtered and concentrated under reduced pressure. The residue was purified by chromatography on silica gel (0-15% MeOH in DCM) to afford (2R)-2-(3-bromophenyl)-2-fluoro-1-(4-methyl-4H-1,2,4-triazol-3-yl)propan-1-ol (675 mg, 50% yield). LCMS (ESI) m/z: 314.0/316.0 [M+H]⁺ (Br pattern).

In step G-4, 0-((2R)-2-(3-bromophenyl)-2-fluoro-1-(4-methyl-4H-1,2,4-triazol-3-yl)propyl) 1H-imidazole-1-carbothioate is made as follows. 1,1′-Thiocarbonyldiimidazole (82 uL, 0.61 mmol) and was added slowly to a solution of (2R)-2-(3-bromophenyl)-2-fluoro-1-(4-methyl-4H-1,2,4-triazol-3-yl)propan-1-ol (160 mg, 0.51 mmol) and DMAP (3.1 mg, 0.030 mmol) in anhydrous DCM (3 mL) at 20° C. The resulting mixture was stirred at 20° C. for 1 h. The reaction was directly purified by chromatography on silica gel (0-10% MeOH in DCM) to afford O-((2R)-2-(3-bromophenyl)-2-fluoro-1-(4-methyl-4H-1,2,4-triazol-3-yl)propyl) 1H-imidazole-1-carbothioate (200 mg, 93% yield). LCMS (ESI) m/z: 424.0/426.0 (Br pattern).

In step G-5, (S)-3-(2-(3-bromophenyl)-2-fluoropropyl)-4-methyl-4H-1,2,4-triazole is made as follows. A mixture of O-((2R)-2-(3-bromophenyl)-2-fluoro-1-(4-methyl-4H-1,2,4-triazol-3-yl)propyl) 1H-imidazole-1-carbothioate (260 mg, 0.61 mmol), tributyltin hydride (1.65 mL, 6.13 mmol and AIBN (15.1 mg, 0.090 mmol) in anhydrous toluene (10 mL) was stirred at 80° C. for 1 h. To the reaction mixture was added potassium fluoride (2 g) and silica gel (20 g) and the resulting mixture was concentrated under reduced pressure. The residue was directly purified by chromatography on silica gel (0-20% MeOH in DCM) to afford (S)-3-(2-(3-bromophenyl)-2-fluoropropyl)-4-methyl-4H-1,2,4-triazole (98 mg, 54% yield). LCMS (ESI) m/z: 298.0/300.0 (Br pattern).

In step G-6, (S)-3-(2-fluoro-1-(4-methyl-4H-1,2,4-triazol-3-yl)propan-2-yl)aniline is made as follows. Copper(I) oxide (28.22 mg, 0.2000 mmol) was added to a solution of 4-methyl-3-[(2S)-2-(3-bromophenyl)-2-fluoro-propyl]-1,2,4-triazole (98 mg, 0.3300 mmol) in conc. aqueous NH₃ (5 mL)/MeCN (2 mL) under nitrogen in a pressure flask. The flask was sealed and the reaction was stirred for 5 h at 110° C. LCMS showed complete conversion with the presence of elimination byproduct. The reaction was diluted with EtOAc/water (20 mL/5 mL). The organic layer were separated, the aqueous phase was extracted with EtOAc (4×20 mL). The combined organic phases were dried with Na₂SO₄, filtered and concentrated under reduced pressure to give the crude product. The crude was purified by flash column chromatography (silica, pretreated with TEA, from 0% MeOH in DCM (0.5% TEA) to 15% MeOH in DCM (0.5% TEA)) to give 4-methyl-3-[(2S)-2-(3-bromophenyl)-2-fluoro-propyl]-1,2,4-triazole (98 mg, 99% yield). LCMS (ESI) [M+H]⁺=235.2.

### Example 8: Intermediate H (6-bromo-2-(3-(3-(fluoro(4-methyl-4H-1,2,4-triazol-3-yl)methyl)oxetan-3-yl)phenyl)-4-(trifluoromethyl)isoindolin-1-one)

Intermediate H can be synthesized according to Scheme 8, FIG. 5H.

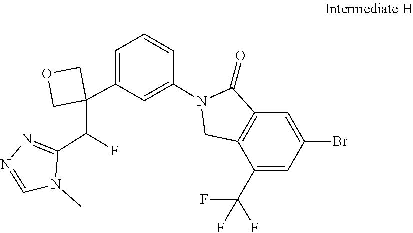 [SMILES: Cn1cnnc1C(F)C1(c2cccc(N3Cc4c(cc(Br)cc4C(F)(F)F)C3=O)c2)COC1]

In a single step, Intermediate H is made as follows. A mixture of 3-[3-[fluoro-(4-methyl-1,2,4-triazol-3-yl)methyl]oxetan-3-yl]aniline (2.00 g, 7.63 mmol) and methyl 5-bromo-2-(bromomethyl)-3-(trifluoromethyl)benzoate (Intermediate A; 2.87 g, 7.63 mmol) in acetonitrile (63.5 mL) and water (31.8 mL) were cooled to 0° C., then a solution of silver nitrate (1.68 g, 9.91 mmol) in water (32 mL) was added drop-wise. The reaction was stirred for 18 h at RT. A saturated solution of sodium bicarbonate was added until the pH reached pH-8. The mixture was then diluted with acetonitrile (5.0 mL), filtered through a short pad of Celite, the cake washed with a 9:1 mixture of CH₂Cl₂/MeOH (500 mL). The cake was washed with more CH₂Cl₂ (700 mL), then pure MeOH until TLC indicates no more product eluted from the Celite cake (˜500 mL). The volatiles were evaporated and the aqueous layer was extracted with CH₂Cl₂ (3×100 mL). The organic phases were combined, washed with brine, dried with sodium sulfate, filtered and concentrated under reduced pressure. The residue was purified by chromatography on silica gel (0-7% methanol in CH₂Cl₂) to afford 6-bromo-2-[3-[3-[fluoro-(4-methyl-1,2,4-triazol-3-yl)methyl]oxetan-3-yl]phenyl]-4-(trifluoromethyl)isoindolin-1-one (1.89 g, 47% yield). LCMS (ESI) m/z: 524.9/526.9 (Br patterns) [M+H]⁺. ¹H NMR (400 MHz, CDCl3) δ 8.20 (d, J=1.2 Hz, 1H), 8.08 (br s, 1H), 7.98 (dd, J=1.6, 0.7 Hz, 1H), 7.61 (d, J=7.5 Hz, 1H), 7.43-7.36 (m, 2H), 6.83 (d, J=7.8 Hz, 1H), 6.46 (d, J=46.0 Hz, 1H), 5.33-5.18 (m, 5H), 5.02-4.80 (m, 3H), 3.08 (d, J=1.5 Hz, 3H).

### Example 9: Intermediate I (2-(3-(3-(fluoro(4-methyl-4H-1,2,4-triazol-3-yl)methyl)oxetan-3-yl)phenyl)-3-oxo-7-(trifluoromethyl)isoindoline-5-carbaldehyde)

Intermediate I can be synthesized according to Scheme 9, FIG. 5I.

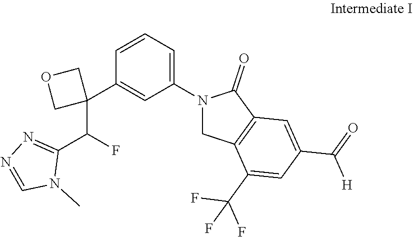 [SMILES: Cn1cnnc1C(F)C1(c2cccc(N3Cc4c(cc(C=O)cc4C(F)(F)F)C3=O)c2)COC1]

In a single step, Intermediate I is made as follows. Following the procedure described for Intermediate H, 2-(3-(3-(fluoro(4-methyl-4H-1,2,4-triazol-3-yl)methyl)oxetan-3-yl)phenyl)-3-oxo-7-(trifluoromethyl)isoindoline-5-carbaldehyde was obtained as an orange solid. LCMS (ESI) m/z: 475.2 [M+H]+. The aldehyde reagent can be made, as described in PCT publication WO2019/14005.

### Example 10: Intermediate J (2-(3-(1,2-Difluoro-1-(4-methyl-4H-1,2,4-triazol-3-yl)-propan-2-yl)phenyl)-3-oxo-7-(trifluoromethyl)isoindoline-5-carbaldehyde)

Intermediate J can be synthesized according to Scheme 10, FIG. 5J.

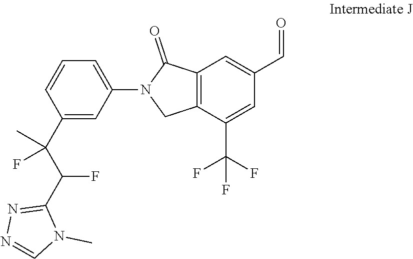 [SMILES: Cn1cnnc1C(F)C(C)(F)c1cccc(N2Cc3c(cc(C=O)cc3C(F)(F)F)C2=O)c1]

In a single step, Intermediate J is made as follows. Following the procedure described for Intermediate H, 2-(3-(1,2-difluoro-1-(4-methyl-4H-1,2,4-triazol-3-yl)propan-2-yl)phenyl)-3-oxo-7-(trifluoromethyl)isoindoline-5-carbaldehyde was obtained as an off-white solid. LCMS (ESI) m/z: 465.0 [M+H]⁺.

### Example 11: Intermediate K (2-(3-(1-(fluoro(4-methyl-4H-1,2,4-triazol-3-yl)methyl)-cyclopropyl)phenyl)-3-oxo-7-(trifluoromethyl)isoindoline-5-carbaldehyde)

Intermediate K can be synthesized according to Scheme 11, FIG. 5K.

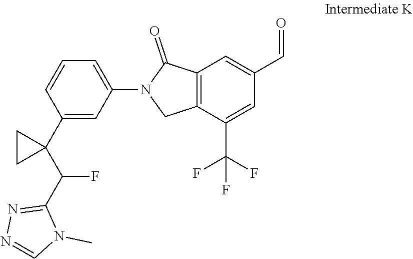 [SMILES: Cn1cnnc1C(F)C1(c2cccc(N3Cc4c(cc(C=O)cc4C(F)(F)F)C3=O)c2)CC1]

In a single step, Intermediate K is made as follows. Following the procedure described for Intermediate H, 2-(3-(1-(fluoro(4-methyl-4H-1,2,4-triazol-3-yl)methyl)cyclo-propyl)phenyl)-3-oxo-7-(trifluoromethyl)isoindoline-5-carbaldehyde was obtained as a yellow solid. LCMS (ESI) m/z: 459.1 [M+H]+.

### Example 12: Intermediate L (2-(3-(1,1-Difluoro-1-(4-methyl-4H-1,2,4-triazol-3-yl)-propan-2-yl)phenyl)-3-oxo-7-(trifluoromethyl)isoindoline-5-carbaldehyde)

Intermediate L can be synthesized according to Scheme 12, FIG. 5L.

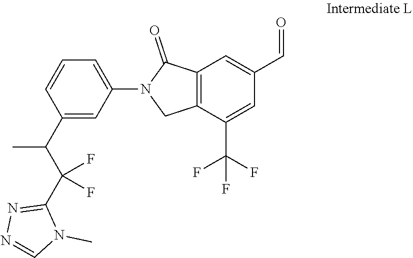 [SMILES: CC(c1cccc(N2Cc3c(cc(C=O)cc3C(F)(F)F)C2=O)c1)C(F)(F)c1nncn1C]

In a single step, Intermediate L is made as follows. Following the procedure described for Intermediate H, 2-(3-(1,1-difluoro-1-(4-methyl-4H-1,2,4-triazol-3-yl)propan-2-yl)phenyl)-3-oxo-7-(trifluoromethyl)isoindoline-5-carbaldehyde was obtained. LCMS (ESI) m/z: 465.1 [M+H]+.

### Example 13: Intermediate M (2-(3-(1-fluoro-1-(4-methyl-4H-1,2,4-triazol-3-yl)propan-2-yl)phenyl)-3-oxo-7-(trifluoromethyl)isoindoline-5-carbaldehyde)

Intermediate M can be synthesized according to Scheme 13, FIG. 5M.

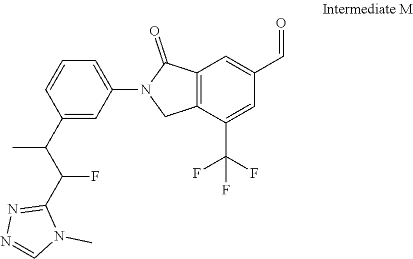 [SMILES: CC(c1cccc(N2Cc3c(cc(C=O)cc3C(F)(F)F)C2=O)c1)C(F)c1nncn1C]

In a single step 1, Intermediate M is made as follows. Following the procedure described for Intermediate H, 2-(3-(1-fluoro-1-(4-methyl-4H-1,2,4-triazol-3-yl)propan-2-yl)phenyl)-3-oxo-7-(trifluoromethyl)isoindoline-5-carbaldehyde. LCMS (ESI) m/z: 447.1 [M+H]+.

### Example 14: Intermediate N ((±)-2-(3-(3,3-difluoro-1-(fluoro(4-methyl-4H-1,2,4-triazol-3-yl)methyl)cyclobutyl)phenyl)-3-oxo-7-(trifluoromethyl)isoindoline-5-carbaldehyde)

Intermediate N can be synthesized according to Scheme 14, FIG. 5N.

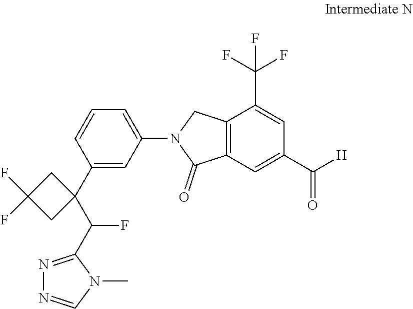 [SMILES: Cn1cnnc1C(F)C1(c2cccc(N3Cc4c(cc(C=O)cc4C(F)(F)F)C3=O)c2)CC(F)(F)C1]

In step N-1, 1-(3-bromophenyl)-3,3-dimethoxycyclobutanecarbonitrile is made as follows. To a mixture of sodium hydride (60%, 5.1 g, 127.5 mmol) in N,N-dimethylformamide (100 mL) was added 2-(3-bromophenyl)acetonitrile (9.9 mL, 51.0 mmol; CAS No.: 539-82-2) at 0° C., the mixture was stirred at 0° C. for 30 minutes. Then 1,3-dibromo-2,2-dimethoxypropane (10.7 g, 40.8 mmol; CAS No.: 22094-18-4) was added at 0° C. The mixture was stirred at 60° C. for 48 h and cooled to room temperature, then the mixture was poured into water (100 mL) and extracted with ethyl acetate (2×150 mL). The combined phases were washed with water (2×100 mL), dried over with anhydrous sodium sulfate and concentrated under vacuum. The residue was purified by silica gel chromatography (mobile phase: ethyl acetate/petroleum ether, gradient 0% to 15%) to afford 1-(3-bromophenyl)-3,3-dimethoxycyclobutanecarbonitrile (7.9 g, 65.4% yield) as a yellow oil. ¹H NMR (400 MHz, CDCl₃): δ 7.63 (t, J=2.0 Hz, 1H), 7.49-7.47 (m, 1H), 7.44-7.41 (m, 1H), 7.31-7.28 (m, 1H), 3.29 (s, 3H), 3.20 (s, 3H), 3.14-3.09 (m, 2H), 2.74-2.69 (m, 2H).

In step N-2, 1-(3-bromophenyl)-3-oxocyclobutanecarbonitrile is made as follows. To a mixture of the 1-(3-bromophenyl)-3,3-dimethoxycyclobutanecarbonitrile (7.9 g, 26.7 mmol) in acetone (40 mL) was added tosylic acid solution (4.0 M, 40.0 mL, 160.0 mmol). The mixture was stirred at 25° C. for 48 h and diluted with water (20 mL). The solution was extracted with ethyl acetate (3×80 mL). The combined organic layers were dried over with anhydrous sodium sulfate and concentrated. The residue was purified by silica gel chromatography (mobile phase: ethyl acetate/petroleum ether, gradient 0% to 15%) to afford 1-(3-bromophenyl)-3-oxocyclobutanecarbonitrile (6.5 g, 97.4% yield) as a light yellow solid. ¹H NMR (400 MHz, CDCl₃): δ 7.65 (t, J=2.0 Hz, 1H), 7.55 (d, J=8.0 Hz, 1H), 7.45-7.43 (m, 1H), 7.36-7.33 (m, 1H), 4.09-4.03 (m, 2H), 3.75-3.68 (m, 2H).

In step N-3, 1-(3-bromophenyl)-3,3-difluorocyclobutanecarbonitrile is made as follows. To a mixture of 1-(3-bromophenyl)-3-oxocyclobutanecarbonitrile (6.5 g, 26.0 mmol) in dichloromethane (150 mL) was added N,N-diethylaminosulfur trifluoride (DAST) (13.9 mL, 104.0 mmol) at 0° C. After addition, the mixture was stirred for 48 h at 25° C. and quenched by addition of saturated aqueous NaHCO₃ solution (30 mL). The mixture was extracted with dichloromethane (3×50 mL). The combined organic layers were dried over with anhydrous sodium sulfate and concentrated. The residue was purified by silica gel chromatography (mobile phase: ethyl acetate/petroleum ether, gradient 0% to 20%) to afford 1-(3-bromophenyl)-3,3-difluorocyclobutanecarbonitrile (6.1 g, 86.3% yield) as a yellow oil. ¹H NMR (400 MHz, CDCl₃): δ 7.62 (s, 1H), 7.55 (d, J=7.6 Hz, 1H), 7.42-7.40 (m, 1H), 7.36-7.32 (m, 1H), 3.56-3.48 (m, 2H), 3.26-3.16 (m, 2H).

In step N-4, 1-(3-bromophenyl)-3,3-difluorocyclobutanecarbaldehyde is made as follows. Diisobutylaluminium hydride (1.0 M in toluene, 26.9 mL, 26.9 mmol) was added to a stirred solution of 1-(3-bromophenyl)-3,3-difluorocyclobutanecarbonitrile (6.1 g, 22.4 mmol) in dichloromethane (60 mL) over 5 minutes at −78° C. under nitrogen protection. The mixture was stirred for 2 h at −78° C. and allowed to warm to 0° C. The mixture was poured into a 100 mL beaker containing ice (20 g) and precooled 1 M HCl aqueous solution (20 mL). The resulting mixture was vigorously stirred for 1 h and then extracted with dichloromethane (3×20 mL). The combined organic layers were dried over with anhydrous sodium sulfate and concentrated under reduced pressure. The residue was purified by silica gel chromatography (mobile phase: ethyl acetate/petroleum ether, gradient 0% to 10%) to afford 1-(3-bromophenyl)-3,3-difluorocyclobutanecarbaldehyde (5 g, 81.1% yield) as a colorless oil. ¹H NMR (400 MHz, CDCl₃): δ 9.54 (s, 1H), 7.50 (d, J=8.0 Hz, 1H), 7.35 (t, J=2.0 Hz, 1H), 7.31 (t, J=8.0 Hz, 1H), 7.13 (d, J=8.0 Hz, 1H), 3.42-3.32 (m, 2H), 3.00-2.90 (m, 2H).

In step N-5, (1-(3-bromophenyl)-3,3-difluorocyclobutyl)(4-methyl-4H-1,2,4-triazol-3-yl)methanol is made as follows. Under nitrogen, to a solution of 4-methyl-1,2,4-triazole (1.96 g, 23.6 mmol; CAS No.: 10570-40-8) in anhydrous 1,2-dimethoxyethane (50 mL) was added n-butyllithium, (2.5 M in hexane, 9.5 mL, 23.6 mmol) at −50° C. over 5 minutes. The resulting mixture was stirred for 1 h at −50° C. and then a solution of 1-(3-bromophenyl)-3,3-difluorocyclo-butanecarbaldehyde (5 g, 18.2 mmol) in 1,2-dimethoxyethane (10 mL) was added dropwise. The reaction mixture was allowed to warm to 0° C. over 1 h and then quenched with water (30 mL). The resulting solution was extracted with dichloromethane (3×50 mL). The combined organic layers were dried over anhydrous sodium sulfate and concentrated under reduced pressure. The residue was purified by silica gel chromatography (mobile phase: methanol/dichloromethane, gradient 0% to 10%) to afford (1-(3-bromophenyl)-3,3-difluorocyclobutyl)(4-methyl-4H-1,2,4-triazol-3-yl)methanol (3.2 g, 98.3% yield) as a white solid. ¹H NMR (400 MHz, methanol-d₄): δ 8.15 (s, 1H), 7.43 (d, J=8.0 Hz, 1H), 7.18 (t, J=8.0 Hz, 1H), 7.10 (s, 1H), 6.91 (d, J=8.0 Hz, 1H), 5.17 (s, 1H), 3.44-3.32 (m, 2H), 3.00-2.73 (m, 5H). LCMS [M+H]+=359.8.

In step N-6, 3-((1-(3-bromophenyl)-3,3-difluorocyclobutyl)fluoromethyl)-4-methyl-4H-1,2,4-triazole is made as follows. Under nitrogen, to a solution of (1-(3-bromophenyl)-3,3-difluorocyclobutyl)(4-methyl-4H-1,2,4-triazol-3-yl)methanol (1 g, 2.8 mmol) in dichloromethane (30 mL) was added N,N-diethylaminosulfur trifluoride (DAST) (1.1 mL, 8.4 mmol) at 0° C. The mixture was stirred for 5 h at 0° C. and then quenched by addition of saturated aqueous NaHCO₃ (10 mL). The resulting mixture was extracted with dichloromethane (3×20 mL). The combined organic layers were dried over anhydrous sodium sulfate and concentrated under reduced pressure to afford a crude product 3-((1-(3-bromophenyl)-3,3-difluorocyclobutyl)fluoromethyl)-4-methyl-4H-1,2,4-triazole (1 g, 99.4% yield) as a yellow solid which was used directly for next step. LCMS [M+H]⁺=361.8.

In step N-7, 3-(3,3-difluoro-1-(fluoro(4-methyl-4H-1,2,4-triazol-3-yl)methyl)cyclobutyl)aniline is made as follows. To a sealed tube was added 3-((1-(3-bromophenyl)-3,3-difluorocyclobutyl)fluoromethyl)-4-methyl-4H-1,2,4-triazole (0.5 g, 1.4 mmol), copper(I) oxide (99.3 mg, 0.7 mmol), ammonia hydroxide (10 mL, 275.3 mmol) and acetonitrile (15 mL). The mixture was stirred at 100° C. for 16 h. The reaction mixture was filtered and concentrated to give crude 3-(3,3-difluoro-1-(fluoro(4-methyl-4H-1,2,4-triazol-3-yl)methyl)cyclobutyl)aniline (400 mg, 97.2% yield) as a light blue solid which was used directly for next step. LCMS: [M+H]⁺=296.9.

In step N-8, 2-(3-(3,3-difluoro-1-(fluoro(4-methyl-4H-1,2,4-triazol-3-yl)methyl)cyclobutyl)phenyl)-3-oxo-7-(trifluoromethyl)isoindoline-5-carbaldehyde is made as follows. To a mixture of methyl 2-(bromomethyl)-5-formyl-3-(trifluoromethyl)benzoate (219.4 mg, 0.7 mmol) and 3-(3,3-difluoro-1-(fluoro(4-methyl-4H-1,2,4-triazol-3-yl)methyl)cyclobutyl)aniline (200.0 mg, 0.7 mmol) in acetonitrile (6 mL) was added a solution of silver nitrate (126.1 mg, 0.7 mmol) in water (2 mL) at 0° C. The mixture was stirred at 25° C. for 16 h and concentrated under vacuum. The residue was dissolved with methanol (10 mL), filtered through a pad of Celite, and rinsed with 10% methanol in dichloromethane (10 mL). The filtrate was concentrated under vacuum. The residue was purified by silica gel chromatography (mobile phase: methanol/dichloromethane, gradient 0% to 6%) to afford 2-2-(3-(3,3-difluoro-1-(fluoro(4-methyl-4H-1,2,4-triazol-3-yl)methyl)cyclobutyl)phenyl)-3-oxo-7-(trifluoromethyl)isoindoline-5-carbaldehyde (200 mg, 58.3% yield) as a yellow solid.

### Example 15: Intermediate P (3-((1-(3-bromophenyl)-3,3-difluorocyclobutyl)methyl)-4-methyl-4H-1,2,4-triazole)

Intermediate P can be synthesized according to Scheme 15, FIG. 5P.

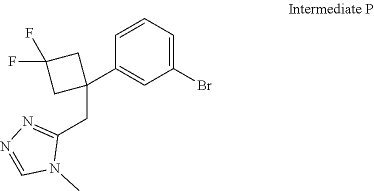 [SMILES: Cn1cnnc1CC1(c2cccc(Br)c2)CC(F)(F)C1]

In Step P-1, 1-(3-bromophenyl)-3,3-difluorocyclobutanecarbaldehyde is made as follows, utilizing starting materials that can be made as shown for the steps to Intermediate N. DIBAL-H, 1.0 M in hexanes (27.6 mL, 27.56 mmol) was added slowly to a solution of 1-(3-bromophenyl)-3,3-difluoro-cyclobutanecarbonitrile (5 g, 18.38 mmol) in diethyl ether (61.3 mL) at −78° C. The resulting mixture was stirred for 2 h at the same temperature. The reaction was carefully quenched with 28 mL of 10% aqueous HCl and allowed to warm to rt. The reaction mixture was diluted with EtOAc and water. The layers were separated, the aqueous phase was extracted with EtOAc (1×), the combined organic phases were washed with saturated aqueous NaHCO₃ solution, water then with brine, dried over magnesium sulfate, filtered and concentrated to afford 1-(3-bromophenyl)-3,3-difluorocyclobutanecarbaldehyde (4.7 g, 93% yield) as an orange oil. ¹H NMR (400 MHz, CDCl₃): δ 9.53 (s, 1H), 7.49 (ddd, J=8.0, 1.8, 1.0 Hz, 1H), 7.34 (t, J=1.9 Hz, 1H), 7.29 (d, J=7.9 Hz, 1H), 7.12 (ddd, J=7.8, 1.7, 1.0 Hz, 1H), 3.42-3.31 (m, 2H), 3.00-2.88 (m, 2H)

In Step P-2, (1-(3-bromophenyl)-3,3-difluorocyclobutyl)(4-methyl-4H-1,2,4-triazol-3-yl)methanol is made as follows. A solution of 2.51 M of n-BuLi M in hexanes (7.49 mL, 18.79 mmol) (titrated) was added dropwise to a solution of 4-methyl-1,2,4-triazole (1.561 g, 18.79 mmol) in anhydrous DME (250 mL) at −50° C. The resulting mixture was stirred at −50° C. for 1 h before a solution of 1-(3-bromophenyl)-3,3-difluorocyclobutanecarbaldehyde (4.7 g, 17.09 mmol) in DME (20 mL+5 mL to wash) was added dropwise. The reaction was gradually allowed to warm to 0° C. over 1 h and quenched with water, diluted with CHCl₃/IPA 4:1 mixture. The layers were separated and the aqueous phase was extracted with CHCl₃/IPA 4:1 mixture (4×). The combined organic phases were concentrated. The residue was triturated into 40 mL of 1:1 DCM/heptanes and the solid was collected by filtration to afford (1-(3-bromophenyl)-3,3-difluorocyclobutyl)(4-methyl-4H-1,2,4-triazol-3-yl)methanol (2.79 g, 46% yield) as a white solid. LCMS (ESI) m/z: 358.0, 359.9 [M+H]⁺

In Step P-3, 3-((1-(3-bromophenyl)-3,3-difluorocyclobutyl)methyl)-4-methyl-4H-1,2,4-triazole is made as follows. A mixture of (1-(3-bromophenyl)-3,3-difluorocyclobutyl)(4-methyl-4H-1,2,4-triazol-3-yl)methanol (2.64 g, 7.37 mmol), thionyl chloride (26.7 mL, 368.53 mmol) and DMF (285.3 μL, 3.69 mmol) was stirred at 60° C. for 30 minutes. The reaction was concentrated under reduced pressure and the residue was co-evaporated with 1:1 DCE/toluene (2×) to afford 3-((1-(3-bromophenyl)-3,3-difluorocyclobutyl)chloromethyl)-4-methyl-4H-1,2,4-triazole as intermediate. The chloro intermediate was treated with zinc dust (72.9 mg, 1.12 mmol) in acetic acid (1.4 mL) and the resulting mixture was stirred at 60° C. for 60 h. The reaction was filtered over Celite, rinsed with 4:1 mixture of CHCl₃/IPA and the filtrate was concentrated. The residue was diluted with saturated aqueous K₂CO₃ solution and 4:1 CHCl₃/IPA. The layers were separated, the aqueous phase was extracted with 4:1 CHCl₃/IPA (2×), the combined organic phases were dried over magnesium sulfate, filtered and concentrated. The crude product was purified by chromatography on silica gel (1-10% of MeOH in DCM) to afford 3-((1-(3-bromophenyl)-3,3-difluorocyclobutyl)methyl)-4-methyl-4H-1,2,4-triazole (2.07 g, 82% yield) as a light yellow solid. LCMS (ESI) m/z: 342.0, 344.0 [M+H]⁺

### Example 16: Intermediate Q (6-(hydroxymethyl)-4-(trifluoromethyl)isoindolin-1-one)

Intermediate Q can be synthesized according to Scheme 16, FIG. 5Q.

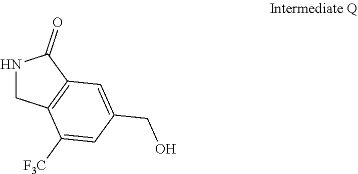 [SMILES: O=C1NCc2c1cc(CO)cc2C(F)(F)F]

In Step Q-1, methyl 2-(bromomethyl)-5-(hydroxymethyl)-3-(trifluoromethyl)-benzoate is made as follows. To a solution of methyl 2-(bromomethyl)-5-formyl-3-(trifluoromethyl)benzoate (5.00 g, 15.38 mmol) in methanol (61.5 mL) at 0° C. was added portion wise and slowly sodium borohydride (640.1 mg, 16.92 mmol). The reaction was stirred at 0° C. for 15 minutes before saturated aqueous NaHCO₃ solution was added. The reaction was allowed to warm to rt and stirred for 10 minutes. The reaction was extracted with EtOAc, dried over sodium sulfate, filtered and concentrated to afford methyl 2-(bromo-methyl)-5-(hydroxymethyl)-3-(trifluoromethyl)benzoate (5.00 g, 99% yield). The crude product will be used like that in next step and yield was assumed quantitative.

In Step Q-2, 6-(hydroxymethyl)-4-(trifluoromethyl)isoindolin-1-one is made as follows. To methyl 2-(bromomethyl)-5-(hydroxymethyl)-3-(trifluoromethyl)benzoate (5.00 g, 15.29 mmol) was added 7 M ammonia solution in MeOH (76.4 mL, 535.02 mmol). The reaction was stirred at rt for 16 h. The reaction mixture was concentrated to get crude 6-(hydroxymethyl)-4-(trifluoromethyl)isoindolin-1-one (3.50 g, 99% yield). Used in the next step without further purification. LCMS (ESI) m/z: 232.3 [M+H]⁺

### Example 17: Intermediate R (tert-butyl (1-methylcyclobutyl)((3-oxo-7-(trifluoro-methyl)isoindolin-5-yl)methyl)carbamate)

Intermediate R can be synthesized according to Schemes 17A and 17B, FIG. 5R.

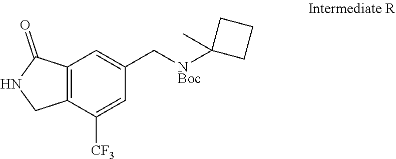 [SMILES: CC(C)(C)OC(=O)N(Cc1cc2c(c(C(F)(F)F)c1)CNC2=O)C1(C)CCC1]

### Scheme 17A

In Step R-1, 6-(chloromethyl)-4-(trifluoromethyl)isoindolin-1-one is made as follows. To a solution of 6-(hydroxymethyl)-4-(trifluoromethyl)isoindolin-1-one (3.50 g, 15.14 mmol) in DCM (75.7 mL) was added thionyl chloride (2.75 mL, 37.85 mmol). The reaction was stirred at rt for 2 days. The reaction was cooled to 0° C. and a few drops of MeOH was added. After 10 minutes the reaction was diluted with saturated aqueous NaHCO₃ solution, extracted with DCM, dried over sodium sulfate, filtered and evaporated to afford 6-(chloromethyl)-4-(trifluoromethyl)isoindolin-1-one (3.705 g, 98% yield). Product was used crude as such in next step. LCMS (ESI) m/z: 250.4 [M+H]⁺

In Step R-2, 6-(((1-methylcyclobutyl)amino)methyl)-4-(trifluoromethyl)isoindolin-1-one is made as follows. To a solution of 1-methylcyclobutanamine; hydrochloride (2.253 g, 18.53 mmol) in MeCN (37.1 mL) was added cesium carbonate (9.719 g, 29.65 mmol). The reaction was stirred at 60° C. for 5 minutes before 6-(chloromethyl)-4-(trifluoromethyl)-isoindolin-1-one (1.850 g, 7.41 mmol) was added. The reaction was stirred at 60° C. for 16 h. The reaction was cooled down to rt and water was added, extracted with EtOAc, combined organic phases were dried over sodium sulfate, filtered and evaporated. The residue was purified by chromatography on silica gel (0-10% MeOH in DCM) to give 6-(((1-methylcyclobutyl)amino)methyl)-4-(trifluoromethyl)isoindolin-1-one (1.230 g, 56% yield). LCMS (ESI) m/z: 299.4 [M+H]⁺.

In Step R-3, tert-butyl (1-methylcyclobutyl)((3-oxo-7-(trifluoromethyl)isoindolin-5-yl)methyl)carbamate is made as follows. To a solution of 6-(((1-methylcyclobutyl)amino)-methyl)-4-(trifluoromethyl)isoindolin-1-one (0.64 g, 2.15 mmol) and di-tert-butyl dicarbonate (0.54 mL, 2.36 mmol) in DCM (7.2 mL) was added triethylamine (0.45 mL, 3.22 mmol). The reaction was stirred at rt for 16 h. The reaction was diluted with saturated aqueous NaHCO₃ solution, extracted with EtOAc, dried over sodium sulfate, filtered and evaporated. The residue was purified by chromatography on silica gel (0-5% MeOH in DCM) to give tert-butyl (1-methylcyclobutyl)((3-oxo-7-(trifluoromethyl)isoindolin-5-yl)methyl)carbamate (745 mg, 87% yield). LCMS (ESI) m/z: 397.4 [M+H]⁺

### Example 18: Intermediate R (Tert-butyl (1-methylcyclobutyl)((3-oxo-7-(trifluoro-methyl)isoindolin-5-yl)methyl)carbamate; route 2)

Intermediate R can be synthesized according to a second route, shown in Scheme 17B, FIG. 5R.

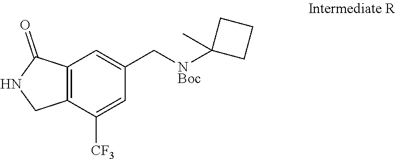 [SMILES: CC(C)(C)OC(=O)N(Cc1cc2c(c(C(F)(F)F)c1)CNC2=O)C1(C)CCC1]

In Step R-4, methyl 2-(bromomethyl)-5-(((1-methylcyclobutyl)amino)methyl)-3-(trifluoromethyl)benzoate is made as follows. To a stirred solution of methyl 2-(bromo-methyl)-5-formyl-3-(trifluoromethyl)benzoate (Intermediate A; 12.0 g, 36.9 mmol) and (1-methylcyclobutyl)ammonium; chloride (4.49 g, 36.9 mmol) in DCE (100 mL) was added triethylamine (5.15 mL, 36.9 mmol) followed by sodium triacetoxyborohydride (23.47 g, 110.74 mmol) portion wise over 20 minutes. The suspension was stirred at rt for 16 h then quenched with addition of saturated aqueous NaHCO₃ solution and extracted with DCM (3×150 mL). The organics were combined, dried over sodium sulfate, filtered and concentrated to afford methyl 2-(bromomethyl)-5-(((1-methylcyclobutyl)amino)methyl)-3-(trifluoromethyl)benzoate (16.2 g, 111% yield) as an amber oil and used in the next step without further purification.

In Step R-5, methyl 2-(bromomethyl)-5-(((tert-butoxycarbonyl)(1-methylcyclo-butyl)amino)methyl)-3-(trifluoromethyl)benzoate is made as follows. To a solution of methyl 2-(bromomethyl)-5-(((1-methylcyclobutyl)amino)methyl)-3-(trifluoromethyl)benzoate (16.5 g, 31.4 mmol) in THF (150 mL) was added triethylamine (13.1 mL, 94.2 mmol) followed by di-tert-butyl dicarbonate (10.8 mL, 47.09 mmol). The reaction was stirred at rt for 16 h. Saturated aqueous NaHCO₃ solution (150 mL) was added and the product was extracted with EtOAc (3×250 mL). The combined organic layers were dried over sodium sulfate, filtered and concentrated to afford methyl 2-(bromomethyl)-5-(((tert-butoxycarbonyl)(1-methylcyclobutyl)amino)methyl)-3-(trifluoromethyl)benzoate (18.2 g, 117% yield). Used in the next step without further purification.

In Step R-6, tert-butyl (1-methylcyclobutyl)((3-oxo-7-(trifluoromethyl)isoindolin-5-yl)methyl)carbamate is made as follows. A solution of methyl 2-(bromomethyl)-5-(((tert-butoxycarbonyl)(1-methylcyclobutyl)amino)methyl)-3-(trifluoromethyl)benzoate (18 g, 16.4 mmol) in 7 M ammonia solution in MeOH (100 mL, 700 mmol) was stirred at rt for 16 h. The reaction was concentrated and the residue was purified by chromatography on silica gel (0-100% EtOAc in heptanes). The product obtained was not clean enough, a second purification was made by chromatography on C18 silica gel (40-100% acetonitrile in ammonium formate buffer, pH=3.7). Appropriate fractions were concentrated, frozen and lyophilized to provide tert-butyl (1-methylcyclobutyl)((3-oxo-7-(trifluoromethyl)isoindolin-5-yl)methyl)carbamate (2.55 g, 39% yield). LCMS (ESI) m/z: 299.2 [M+H]⁺.

### Example 19: Intermediate S ((S)-6-((2-isopropyl-4-methylpiperazin-1-yl)methyl)-4-(trifluoromethyl)isoindolin-1-one)

Intermediate S can be synthesized according to Scheme 18, FIG. 5S.

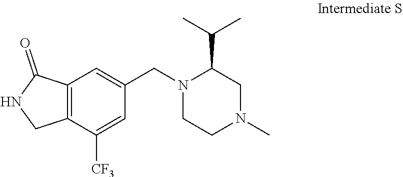 [SMILES: CC(C)[C@H]1CN(C)CCN1Cc1cc2c(c(C(F)(F)F)c1)CNC2=O]

In Step S-1, (S)-methyl 2-(bromomethyl)-5-((2-isopropyl-4-methylpiperazin-1-yl)methyl)-3-(trifluoromethyl)benzoate is made as follows. To a stirred solution of methyl 2-(bromomethyl)-5-formyl-3-(trifluoromethyl)benzoate (550 mg, 1.61 mmol), (3S)-3-isopropyl-1-methyl-piperazine (229.3 mg, 1.61 mmol) in DCE (15 mL) was added portion wise sodium triacetoxyborohydride (1.025 g, 4.84 mmol) over 10 minutes, then the suspension was stirred at rt for 20 h. The reaction mixture was quenched with addition of saturated aqueous NaHCO₃ solution and extracted with CHCl₃: IPA (9:1) (3×). Combined organic layers were dried over sodium sulfate, filtered and concentrated to provide (S)-methyl 2-(bromomethyl)-5-((2-isopropyl-4-methylpiperazin-1-yl)methyl)-3-(trifluoromethyl)benzoate (728 mg, 100% yield) which was used in next step without any purification. LCMS (ESI) m/z: 451.0, 452.9 [M+H]⁺

In Step S-2, (S)-6-((2-isopropyl-4-methylpiperazin-1-yl)methyl)-4-(trifluoromethyl)-isoindolin-1-one is made as follows. A solution of (S)-methyl 2-(bromomethyl)-5-((2-isopropyl-4-methylpiperazin-1-yl)methyl)-3-(trifluoromethyl)benzoate (728 mg, 1.61 mmol), in 7 M ammonia in MeOH (10 mL, 70 mmol) was stirred at rt for 3 h. The reaction was concentrated and the crude residue was purified by chromatography on C18 silica gel (0-100% acetonitrile in ammonium formate buffer, pH=3.8). Appropriate fractions were concentrated, frozen and lyophilized to afford (S)-6-((2-isopropyl-4-methylpiperazin-1-yl)methyl)-4-(trifluoromethyl)isoindolin-1-one (120 mg, 21% yield) as a white solid. LCMS (ESI) m/z: 356.2 [M+H]⁺

### Example 20: Intermediate T ((S)-tert-butyl 3-isopropyl-4-((3-oxo-7-(trifluoromethyl)-isoindolin-5-yl)methyl)piperazine-1-carboxylate)

Intermediate T can be synthesized according to Scheme 19, FIG. 5T.

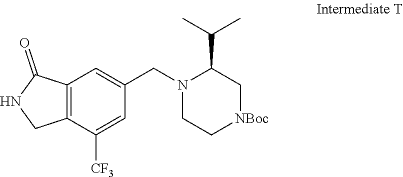 [SMILES: CC(C)[C@H]1CN(C(=O)OC(C)(C)C)CCN1Cc1cc2c(c(C(F)(F)F)c1)CNC2=O]

In Step T-1, (S)-tert-butyl 4-(4-(bromomethyl)-3-(methoxycarbonyl)-5-(trifluoromethyl)benzyl)-3-isopropylpiperazine-1-carboxylate is made as follows. To a stirred solution of methyl 2-(bromomethyl)-5-formyl-3-(trifluoromethyl)benzoate (4.2 g, 12.92 mmol) and tert-butyl (3S)-3-isopropylpiperazine-1-carboxylate (2.95 g, 12.92 mmol) in DCE (64.6 mL) was added sodium triacetoxyborohydride (8.21 g, 38.76 mmol) portion wise over 20 minutes, then the suspension was stirred at rt for 16 h. The reaction was quenched with addition of saturated aqueous NaHCO₃ solution and extracted with DCM (3×). The combined organics were dried over sodium sulfate, filtered and evaporated to afford (S)-tert-butyl 4-(4-(bromomethyl)-3-(methoxycarbonyl)-5-(trifluoromethyl)benzyl)-3-isopropylpiperazine-1-carboxylate, assumed quantitative and used as is in the next step. LCMS (ESI) m/z: 536.9, 538.9 [M+H]⁺

In Step T-2, (S)-tert-butyl 3-isopropyl-4-((3-oxo-7-(trifluoromethyl)isoindolin-5-yl)methyl)piperazine-1-carboxylate is made as follows. A solution of (S)-tert-butyl 4-(4-(bromomethyl)-3-(methoxycarbonyl)-5-(trifluoromethyl)benzyl)-3-isopropylpiperazine-1-carboxylate (6.94 g, 12.92 mmol), in 7 M ammonia solution in MeOH (36.9 mL, 258.4 mmol) was stirred at rt for 16 h. The reaction was concentrated and the residue was purified by chromatography on C18 silica gel (0-100% acetonitrile in ammonium formate buffer, pH=3.8). Appropriate fractions were concentrated, frozen and lyophilized to give (S)-tert-butyl 3-isopropyl-4-((3-oxo-7-(trifluoromethyl)isoindolin-5-yl)methyl)piperazine-1-carboxylate (1.66 g, 29% yield over two steps) as a white solid. LCMS (ESI) m/z: 442.0 [M+H]⁺

### Example 21: Intermediate U ((1-(2,6-dichloropyridin-4-yl)cyclobutyl)(4-methyl-4H-1,2,4-triazol-3-yl)methanol)

Intermediate U can be synthesized according to Scheme 20, FIG. 5U.

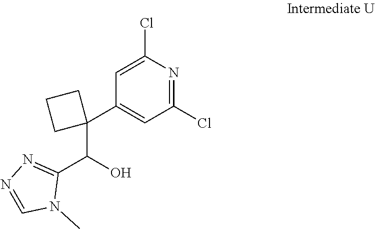 [SMILES: Cn1cnnc1C(O)C1(c2cc(Cl)nc(Cl)c2)CCC1]

In Step U-1, 1-(2,6-dichloropyridin-4-yl)cyclobutanecarbonitrile is made as follows. 2,4,6-trichloropyridine (5 g, 27.41 mmol) and cyclobutanecarbonitrile (2.54 mL, 28.78 mmol) was dissolved in THF (54.8 mL) and cooled to −78° C. 1 M LiHMDS solution in THF (30.2 mL, 30.15 mmol) was added dropwise over 10 minutes. Cooling bath was removed and reaction mixture was allowed to warm to rt and stirred for 2 h. The reaction was quenched by addition of saturated aqueous NH₄Cl solution, extracted with DCM (3×), combined organic layers were washed with brine and dried over sodium sulfate, filtered and concentrated. The crude product was purified by chromatography on silica gel (0-50% of EtOAc in heptanes) to afford 1-(2,6-dichloropyridin-4-yl)cyclobutanecarbonitrile (4.5 g, 72% yield). LCMS (ESI) m/z: 227.3, 229.1 [M+H]⁺ ¹H NMR (400 MHz, CDCl₃) δ 7.33 (s, 2H), 2.94-2.77 (m, 2H), 2.67-2.40 (m, 3H), 2.24-2.05 (m, 1H)

In Step U-2, 1-(2,6-dichloropyridin-4-yl)cyclobutanecarbaldehyde is made as follows. 1 M DIBAL-H solution in toluene (21.8 mL, 21.8 mmol) was added slowly to a solution of 1-(2,6-dichloropyridin-4-yl)cyclobutanecarbonitrile (4.5 g, 19.82 mmol) in toluene (116.7 mL) at −78° C. The resulting mixture was stirred for 1 h at −78° C. The reaction was quenched by the addition of saturated aqueous NH₄Cl solution, extracted with DCM (3×). The combined organic layers were washed with brine, dried over sodium sulfate, filtered and concentrated. The crude product was purified by chromatography on silica gel (0-50% of EtOAc in heptanes) to afford 1-(2,6-dichloropyridin-4-yl)cyclobutanecarbaldehyde (2.5 g, 55% yield). LCMS (ESI) m/z: 230.1, 232.1 [M+H]⁺ ¹H NMR (400 MHz, CDCl₃) δ 9.61 (s, 1H), 7.04 (s, 2H), 2.76 (m, 2H), 2.43 (m, 2H), 2.21-1.91 (m, 2H).

In Step U-3, (1-(2,6-dichloropyridin-4-yl)cyclobutyl)(4-methyl-4H-1,2,4-triazol-3-yl)methanol is made as follows. At −50° C., 2.5 M n-BuLi solution in hexanes (5.0 mL, 12.52 mmol) was added to a solution of 4-methyl-1,2,4-triazole (1.04 g, 12.52 mmol) in DME (120 ml) and the reaction was stirred at −50° C. for 1 h. 1-(2,6-dichloropyridin-4-yl)cyclobutane-carbaldehyde (2.4 g, 10.43 mmol) in DME (30 mL) was added dropwise and the reaction was allowed to slowly warm to rt. After 1 h at rt the reaction was quenched with saturated aqueous NH₄Cl solution and extracted with 4:1 mixture of CHCl₃/IPA (3×500 mL). The combined organic layers were dried over sodium sulfate, filtered and evaporated. The crude product was purified by chromatography on silica gel (0-15% of MeOH in DCM) to provide (1-(2,6-dichloropyridin-4-yl)cyclobutyl)(4-methyl-4H-1,2,4-triazol-3-yl)methanol (0.820 g, 25% yield). LCMS (ESI) m/z: 313.1, 315.2 [M+H]⁺

### Example 22: Intermediate V (2,6-dichloro-4-(1-((4-methyl-4H-1,2,4-triazol-3-yl)methyl)-cyclobutyl)pyridine)

Intermediate V can be synthesized according to Scheme 21, FIG. 5V.

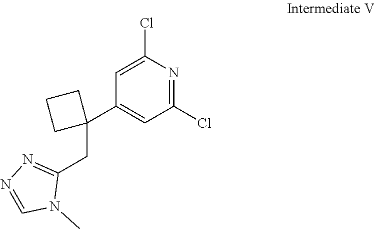 [SMILES: Cn1cnnc1CC1(c2cc(Cl)nc(Cl)c2)CCC1]

In Step V-1, 2,6-dichloro-4-(1-(chloro(4-methyl-4H-1,2,4-triazol-3-yl)methyl)-cyclobutyl)pyridine is made as follows. (1-(2,6-dichloropyridin-4-yl)cyclobutyl)(4-methyl-4H-1,2,4-triazol-3-yl)methanol (9.58 g, 30.59 mmol) was suspended in DCM (305.9 mL), then thionyl chloride (4.4 mL, 61.18 mmol) and a catalytic amount of DMF (˜5 drops) were added. The solution was stirred at rt for 3.5 h, then was quenched with water and extracted with a 4:1 mixture of CHCl₃: IPA (4×). The combined organic phases were dried over sodium sulfate, filtered and evaporated. The crude mixture was purified by trituration with EtOAc and the solid was collected by filtration, washed with a small volume of EtOAc, then with heptanes and dried under high vacuum to afford 2,6-dichloro-4-(1-(chloro(4-methyl-4H-1,2,4-triazol-3-yl)methyl)cyclobutyl)pyridine (7.16 g, 71% yield) as a white solid. LCMS (ESI) m/z: 331.0, 333.0, 335.0 [M+H]⁺.

In Step V-2, 2,6-dichloro-4-(1-((4-methyl-4H-1,2,4-triazol-3-yl)methyl)cyclobutyl)-pyridine is made as follows. 2,6-dichloro-4-(1-(chloro(4-methyl-4H-1,2,4-triazol-3-yl)methyl)-cyclobutyl)pyridine (7.16 g, 21.59 mmol) was suspended in acetic acid (72 mL) and zinc (2.12 g, 32.39 mmol) was added. The mixture was stirred vigorously at 55° C. for 1 h then cooled to rt and most of the acetic acid was evaporated. The excess zinc was quenched with addition of 1 N HCl solution, then stirred and sonicated for a few minutes. The solution was basified with 5 N NaOH solution and diluted with a 4:1 mixture of CHCl₃: IPA, the layers were separated and the organics were washed with 1 N NaOH solution (2×). All the aqueous layers were combined and extracted with CHCl₃: IPA (5×). The organics were combined, dried over sodium sulfate, filtered and evaporated to yield 2,6-dichloro-4-(1-((4-methyl-4H-1,2,4-triazol-3-yl)methyl)cyclobutyl)pyridine (6.51 g, 101% yield) as a white solid which was used in next step without further purification. LCMS (ESI) m/z: 297.0, 299.0 [M+H]⁺ ¹H NMR (400 MHz, DMSO-d6) δ 8.26 (s, 1H), 7.26 (s, 2H), 3.30 (s, 2H), 3.27 (s, 3H), 2.45-2.32 (m, 4H), 2.15-2.02 (m, 1H), 1.86-1.75 (m, 1H).

### Example 23: Intermediate W (3-(3,3-difluoro-1-((4-methyl-4H-1,2,4-triazol-3-yl)methyl)-cyclobutyl)aniline)

Intermediate W can be synthesized according to Scheme 22, FIG. 5W.

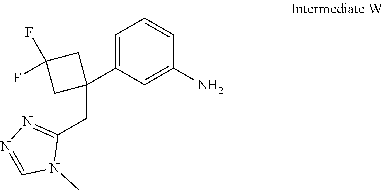 [SMILES: Cn1cnnc1CC1(c2cccc(N)c2)CC(F)(F)C1]

In a microwave vial, copper(I) oxide (250.9 mg, 1.75 mmol) was added to a mixture of 3-((1-(3-bromophenyl)-3,3-difluorocyclobutyl)methyl)-4-methyl-4H-1,2,4-triazole (1 g, 2.92 mmol) and concentrated aqueous NH₃ solution (6.mL) in MeCN (5.8 mL) under nitrogen. The vial was sealed and the reaction was stirred for 16 h at 100° C. The reaction was cooled to rt and filtered through a pad of Celite and washed with MeOH and water. The filtrate was diluted with 4:1 CHCl₃/IPA and brine. The layers were separated, the aqueous phase was extracted with 4:1 CHCl₃/IPA (3×50 mL), and the combined organic phases were dried over sodium sulfate, filtered and concentrated under reduced pressure. The crude residue was purified by chromatography on C18 silica gel (0-50% acetonitrile in ammonium formate buffer, pH=3.8). Appropriate fractions were concentrated, frozen and lyophilized to afford 3-(3,3-difluoro-1-((4-methyl-4H-1,2,4-triazol-3-yl)methyl)cyclobutyl)-aniline (653 mg, 80% yield) as a green solid. LCMS (ESI) m/z: 279.3 [M+H]⁺.

### Example 24: Intermediate X (4-(difluoromethyl)-6-(hydroxymethyl)isoindolin-1-one)

Intermediate X can be synthesized according to Scheme 23, FIG. 5X.

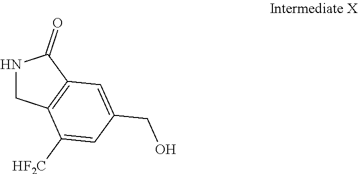

In Step X-1, 1-bromo-3-(difluoromethyl)-2-methylbenzene is synthesized as follows. To a solution of 3-bromo-2-methyl-benzaldehyde (3.500 g, 17.58 mmol) in DCM (5 mL) at 0° C. was added DAST (9.3 mL, 70.34 mmol). The reaction was stirred at for 2 days. The reaction was diluted with water and extracted with DCM. The combined organics layers were washed with saturated aqueous NaHCO₃ solution, dried over sodium sulfate, filtered and evaporated. The combined organic layers were dried over sodium sulfate, filtered and concentrated. The crude was purified by chromatography on silica gel (0-20% EtOAc in heptane) to obtain 1-bromo-3-(difluoromethyl)-2-methyl-benzene (3.36 g, 86% yield).

In Step X-2, 3-(difluoromethyl)-2-methylbenzoic acid is synthesized as follows. A flask was charged with 1-bromo-3-(difluoromethyl)-2-methyl-benzene (3.54 mg, 16.02 mmol), palladium acetate (719.1 mg, 3.2 mmol), XantPhos (926.7 mg, 1.6 mmol) and oxalic acid (2.18 mL, 24.02 mmol). After purging with nitrogen, degassed DMF (53.4 mL) containing DIPEA (4.2 mL, 24.02 mmol) and acetic anhydride (2.3 mL, 24.02 mmol) was added and the reaction heated to 100° C. for 16 h. The reaction mixture was extracted with 1 M NaOH solution (2×). The aqueous phases were washed with EtOAc (2×) and acidified with HCl concentrated. The aqueous phases was extracted with EtOAc, washed with brine, dried over sodium sulfate, filtered and evaporated to afford crude 3-(difluoromethyl)-2-methyl-benzoic acid (1.2 g, 40% yield) which was used as such in next step. LCMS (ESI) m/z: 185.0 [M−H]⁻.

In Step X-3, methyl 3-(difluoromethyl)-2-methylbenzoate is synthesized as follows. To a solution of 3-(difluoromethyl)-2-methyl-benzoic acid (1.2 g, 6.45 mmol) in DMF (12.9 mL) was added potassium carbonate (2.672 g, 19.34 mmol) at 0° C. After 10 minutes iodomethane (802.6 μL, 12.89 mmol) was added and the reaction was stirred at rt for 16 h. The reaction was diluted with saturated aqueous NaHCO₃, extracted with EtOAc, washed with brine, dried over sodium sulfate, filtered and evaporated to afford methyl 3-(difluoromethyl)-2-methyl-benzoate (1.28 g, 99% yield). The residue was used crude as such in next step.

In Step X-4, methyl 5-bromo-3-(difluoromethyl)-2-methylbenzoate is synthesized as follows. To a solution of methyl 3-(difluoromethyl)-2-methyl-benzoate (1.28 g, 6.39 mmol) in acetic acid (8.9 mL) were added 70% HNO₃ solution in water, (2.9 mL, 63.94 mmol) and bromine (360.3 μL, 7.03 mmol) followed by dropwise addition of 2.5 M AgNO₃ solution in water (3.33 mL, 8.31 mmol) using an addition funnel. The mixture was then stirred at rt for 16 h. The reaction mixture was then poured on ice, diluted with saturated aqueous Na₂CO₃ solution and extracted with EtOAc (3×). The organics were combined, washed with water (3×), dried over sodium sulfate, filtered and evaporated. The crude was purified by chromatography on silica gel (0-20% EtOAc in heptane) to obtain methyl 5-bromo-3-(difluoromethyl)-2-methyl-benzoate (1.435 g, 80% yield).

In Step X-5, 3-(difluoromethyl)-5-(methoxycarbonyl)-4-methylbenzoic acid is synthesized as follows. A flask was charged with methyl 5-bromo-3-(difluoromethyl)-2-methyl-benzoate (1.430 g, 5.12 mmol), palladium acetate (230.1 g, 1.02 mmol), XantPhos (296.5 mg, 0.51 mmol) and oxalic acid (699 μL, 7.69 mmol). After purging with nitrogen, degassed DMF (25.6 mL) containing DIPEA (1.34 mL, 7.69 mmol) and acetic anhydride (725.2 μL, 7.69 mmol) was added and the reaction heated to 100° C. for 16 h. The reaction mixture was extracted with saturated aqueous Na₂CO₃ solution (2×) and washed with EtOAc (2×). The aqueous phases were acidified with HCl concentrated, extracted with EtOAc, washed with brine, dried over sodium sulfate, filtered and evaporated. The crude residue was purified by chromatography on C18 silica gel (15-45% acetonitrile in ammonium formate buffer, pH=3.8) Only the cleanest fraction were collected frozen and lyophilized to afford 3-(difluoromethyl)-5-methoxycarbonyl-4-methyl-benzoic acid (833 mg, 67% yield). LCMS (ESI) m/z: 243.0 [M−H]⁻.

In Step X-6, 4-(bromomethyl)-3-(difluoromethyl)-5-(methoxycarbonyl)benzoic acid is synthesized as follows. To a solution of 3-(difluoromethyl)-5-methoxycarbonyl-4-methyl-benzoic acid (830 mg, 3.4 mmol) in carbon tetrachloride (22.7 mL) was added N-bromosuccinimide (907.5 mg, 5.1 mmol) and benzoyl peroxide (247 mg, 1.02 mmol). The reaction was stirred at 80° C. for 16 h. The reaction was extracted with saturated aqueous Na₂CO₃ solution and the combined aqueous washed with DCM. The aqueous was acidified with 3 N HCl solution, extracted with EtOAc, dried over sodium sulfate, filtered and evaporated to afford 4-(bromomethyl)-3-(difluoromethyl)-5-(methoxycarbonyl)benzoic acid (1.029 g, 94% yield). The residue was used like that in next step. LCMS (ESI) m/z: 321.0 [M+H]⁺.

In Step X-7, methyl 2-(bromomethyl)-3-(difluoromethyl)-5-(hydroxymethyl)-benzoate is synthesized as follows. To a solution of 4-(bromomethyl)-3-(difluoromethyl)-5-methoxycarbonyl-benzoic acid (300 mg, 0.93 mmol) in THF (3.7 mL) was added 1 M borane solution in THF (3.3 mL, 3.25 mmol) at 0° C. The reaction was stirred at rt for 16 h. The reaction was diluted with saturated aqueous NaHCO₃ solution, extracted with EtOAc, dried over sodium sulfate, filtered and evaporated. The crude was purified by chromatography on silica gel (0-30% EtOAc in heptanes) to obtain methyl 2-(bromomethyl)-3-(difluoromethyl)-5-(hydroxymethyl)benzoate (211.1 mg, 74% yield) ¹H NMR (400 MHz, CDCl₃) δ 8.05 (s, 1H), 7.80 (s, 1H), 7.06 (t, J=54.8 Hz, 1H), 5.06 (s, 2H), 4.79 (s, 2H), 3.97 (s, J=0.8 Hz, 3H).

In Step X-8, 4-(difluoromethyl)-6-(hydroxymethyl)isoindolin-1-one is synthesized as follows. To methyl 2-(bromomethyl)-3-(difluoromethyl)-5-(hydroxymethyl)benzoate (485 mg, 1.57 mmol) was added 7 M ammonia solution in MeOH (10.1 mL, 70.61 mmol). The reaction was stirred at rt for 16 h. After completion, the reaction mixture was concentrated. A minimum of water was added and the aqueous was extracted several time with Me-THF, dried over sodium sulfate, filtered and evaporated to give the crude product used like that in next step. LCMS (ESI) m/z: 214.2 [M+H]⁺.

Examples 30 to 80 describe synthesis of various exemplary isoindolin-1-one compounds according to the invention. In some instances in which a mixture of diastereomeric or enantiomeric compounds were made, the stereochemistry has only been arbitrarily assigned to one or other of the compounds.

### Example 25: Compounds 9 and 10

Compounds 9 and 10, stereoisomers of one another, can be synthesized according to Scheme 24, FIG. 6.

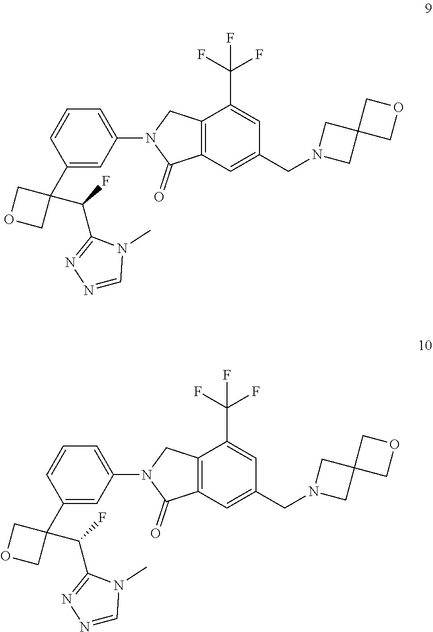 [SMILES: Cn1cnnc1[C@H](F)C1(c2cccc(N3Cc4c(cc(CN5CC6(COC6)C5)cc4C(F)(F)F)C3=O)c2)COC1 ; Cn1cnnc1[C@@H](F)C1(c2cccc(N3Cc4c(cc(CN5CC6(COC6)C5)cc4C(F)(F)F)C3=O)c2)COC1]

To a solution of 2-(3-(3-(fluoro(4-methyl-4H-1,2,4-triazol-3-yl)methyl)oxetan-3-yl)phenyl)-3-oxo-7-(trifluoromethyl)isoindoline-5-carbaldehyde (Intermediate I, Scheme 9, FIG. 6I. 300.0 mg, 0.65 mmol) in methanol (9 mL) was added 2-oxa-6-azaspiro[3.3]heptane oxalate (370.8 mg, 1.96 mmol) and triethylamine (0.25 mL, 1.83 mmol). The mixture was heated under micromave irradiation at 100° C. for 1 minute and cooled down to room temperature. The mixture was then added sodium cyanoborohydride (43.7 mg, 0.70 mmol) and heated at 80° C. under microwave irradiation for another 45 minutes. The reaction was quenched with 1 M HCl solution and the result solution was adjusted to pH=7 by addition of saturated NaHCO₃ solution. The resulting solution was extracted with dichloromethane (3×20 mL). The combined organic phases were dried over anhydrous sodium sulphate and concentrated under reduced pressure. The residue was purified by preparative TLC (solvent gradient: 10% methanol in dichloromethane) to afford 6-((2-oxa-6-azaspiro[3.3]heptan-6-yl)methyl)-2-(3-(3-(fluoro(4-methyl-4H-1,2,4-triazol-3-yl)methyl)oxetan-3-yl)phenyl)-4-(trifluoromethyl)isoindolin-1-one (60.0 mg, 17% yield) as a colourless oil. LCMS [M+H]⁺=558.1.

The above racemate (60.0 mg, 0.11 mmol) was further purified by chiral SFC (Column=Daicel Chiralcel OD-H; Column dimensions=250 mm×30 mm×5 μm; Detection wavelength=220 nM; Flow rate=60 mL/min; Run time=4.5 min; Column temperature=25° C.) with 0.1% ammonium hydroxide-45% ethanol-carbon dioxide) to afford Compounds 9 and 10, characterized as follows.

(R)-6-(2-oxa-6-azaspiro[3.3]heptan-6-ylmethyl)-2-(3-(3-(fluoro(4-methyl-4H-1,2,4-triazol-3-yl)methyl)oxetan-3-yl)phenyl)-4-(trifluoromethyl)isoindolin-1-one (Peak 1, retention time=2.122 min) (2.6 mg, 4% yield) as a white solid (9). ¹H NMR (400 MHz, methanol-d₄): δ 8.28 (s, 1H), 8.01 (s, 1H), 7.91 (s, 1H), 7.80 (dd, J=1.6, 8.0 Hz, 1H), 7.52 (t, J=2.0 Hz, 1H), 7.43 (t, J=8.0 Hz, 1H), 6.93 (d, J=8.0 Hz, 1H), 6.31 (d, J=45.6 Hz, 1H), 5.48 (d, J=6.8 Hz, 1H), 5.31 (d, J=6.8 Hz, 1H), 5.22 (dd, J=1.6, 6.4 Hz, 1H), 5.10 (br. s, 2H), 5.02 (dd, J=4.4, 6.4 Hz, 1H), 4.76 (s, 4H), 3.84 (s, 2H), 3.56 (s, 4H), 3.12 (s, 3H).

(S)-6-(2-oxa-6-azaspiro[3.3]heptan-6-ylmethyl)-2-(3-(3-(fluoro(4-methyl-4H-1,2,4-triazol-3-yl)methyl)oxetan-3-yl)phenyl)-4-(trifluoromethyl)isoindolin-1-one (Peak 2, retention time=2.926 min) (3.5 mg, 6% yield) as a white solid (10). ¹H NMR (400 MHz, methanol-d₄): δ 8.28 (s, 1H), 8.01 (s, 1H), 7.91 (s, 1H), 7.80 (dd, J=1.6, 8.0 Hz, 1H), 7.52 (t, J=2.0 Hz, 1H), 7.43 (t, J=8.0 Hz, 1H), 6.93 (d, J=8.0 Hz, 1H), 6.31 (d, J=45.6 Hz, 1H), 5.48 (d, J=6.8 Hz, 1H), 5.31 (d, J=6.0 Hz, 1H), 5.22 (dd, J=1.2, 6.4 Hz, 1H), 5.10 (br. s, 2H), 5.02 (dd, J=4.0, 6.4 Hz, 1H), 4.76 (s, 4H), 3.83 (s, 2H), 3.55 (s, 4H), 3.12 (s, 3H). LCMS [M+H]⁺ or [M−H]⁻: 558.2.

### Example 26: Compound 11

Compound 11 can be synthesized according to Scheme 25, FIG. 7.

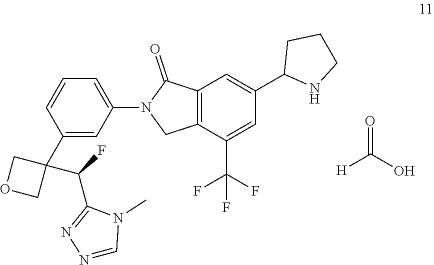 [SMILES: Cn1cnnc1[C@H](F)C1(c2cccc(N3Cc4c(cc(C5CCCN5)cc4C(F)(F)F)C3=O)c2)COC1 ; O=CO]

An intermediate, tert-butyl 2-(2-(3-(3-((4-methyl-4H-1,2,4-triazol-3-yl)methyl)oxetan-3-yl)phenyl)-3-oxo-7-(trifluoromethyl)isoindolin-5-yl)pyrrolidine-1-carboxylate, is made in the following step. A mixture of 6-bromo-2-(3-(3-((4-methyl-4H-1,2,4-triazol-3-yl)methyl)oxetan-3-yl)phenyl)-4-(trifluoromethyl)isoindolin-1-one (synthesized as described in WO2019148005, 100.0 mg, 0.200 mmol), nickel (II) chloride ethylene glycol dimethyl ether complex (4.3 mg, 0.02 mmol), 1-tert-butoxycarbonylpyrrolidine-2-carboxylic acid (127.3 mg, 0.59 mmol) in N,N-dimethylformamide (2 mL) was added bis[3,5-difluoro-2-[5-(trifluoromethyl)-2-pyridyl]phenyl]iridium-4-tert-butyl-2-(4-tert-butyl-2-pyridyl)pyridine hexafluorophosphate (6.6 mg, 0.01 mmol), cesium carbonate (192.7 mg, 0.59 mmol) and 4,4′-di-tert-butyl-2,2′-dipyridyl (7.9 mg, 0.03 mmol) in glove box at 20° C. The reaction mixture was stirred under a Lumidox Screen Kit for 20 h at 20° C. and diluted with water (5 mL). The mixture was extracted with ethyl acetate (3×20 mL). The combined organic phases were dried over sodium sulfate and concentrated under reduced pressure. The residue was purified by preparative TLC (mobile phase: 12% methanol in ethyl acetate) to give tert-butyl 2-(2-(3-(3-((4-methyl-4H-1,2,4-triazol-3-yl)methyl)oxetan-3-yl)phenyl)-3-oxo-7-(trifluoromethyl)isoindolin-5-yl)pyrrolidine-1-carboxylate (50.0 mg, 21% yield) as a yellow solid. LCMS [M+H]⁺=598.2.

To a mixture of tert-butyl 2-[2-[3-[3-[(4-methyl-1,2,4-triazol-3-yl)methyl]oxetan-3-yl]phenyl]-3-oxo-7-(trifluoromethyl)isoindolin-5-yl]pyrrolidine-1-carboxylate (50.0 mg, 0.08 mmol) in dichloromethane (4 mL) was added trifluoroacetic acid (0.2 mL, 0.08 mmol). The mixture was stirred at 18° C. for 3 h and concentrated under reduced pressure. The resulting residue was purified by RP-HPLC (10% to 40% ACN/(0.2% formic acid in water)) to afford 2-(3-(3-((4-methyl-4H-1,2,4-triazol-3-yl)methyl)oxetan-3-yl)phenyl)-6-(pyrrolidin-2-yl)-4-(trifluoromethyl)isoindolin-1-one formate (11) (6.7 mg, 14% yield) as a yellow solid. ¹H NMR (400 MHz, methanol-d₄): δ 8.32-8.13 (m, 3H), 8.08 (s, 1H), 7.74 (d, J=8.0 Hz, 1H), 7.43-7.33 (m, 2H), 6.77 (d, J=7.6 Hz, 1H), 5.13 (s, 2H), 5.07-5.01 (m, 4H), 3.63 (s, 2H), 3.57-3.43 (m, 2H), 2.88 (s, 3H), 2.64-2.53 (m, 1H), 2.37-2.17 (m, 3H), 1.57-1.32 (m, 1H). LCMS [M+H]⁺ or [M−H]⁻: 498.2.

### Example 27: Compound 12

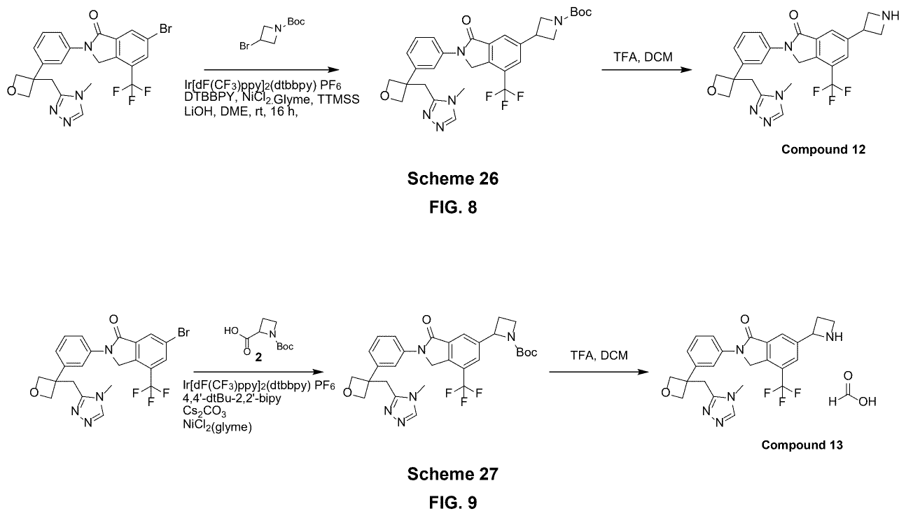

Compound 12 can be synthesized according to Scheme 26, FIG. 8.

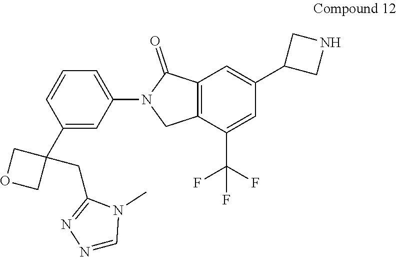 [SMILES: Cn1cnnc1CC1(c2cccc(N3Cc4c(cc(C5CNC5)cc4C(F)(F)F)C3=O)c2)COC1]

An intermediate, tert-butyl 3-(2-(3-(3-((4-methyl-4H-1,2,4-triazol-3-yl)methyl)oxetan-3-yl)phenyl)-3-oxo-7-(trifluoromethyl)isoindolin-5-yl)azetidine-1-carboxylate, can be synthesized as follows. To a mixture of tert-butyl 3-bromoazetidine-1-carboxylate (139.6 mg, 0.59 mmol), 6-bromo-2-(3-(3-((4-methyl-4H-1,2,4-triazol-3-yl)methyl)oxetan-3-yl)phenyl)-4-(trifluoromethyl)isoindolin-1-one (synthesized as described in WO2019148005, 100.0 mg, 0.20 mmol), in 1,2-dimethoxyethane (2.5 mL) was added hydroxylithium (11.8 mg, 0.49 mmol), Ir[dF(CF₃)ppy]₂(dtbbpy)PF₆ (2.21 mg, 0.005 mmol), tris(trimethylsilyl)silane (58.8 mg, 0.24 mmol). Then a solution of 4,4′-di-tert-butyl-2,2′-dipyridyl (7.9 mg, 0.03 mmol) and nickel(II) chloride ethylene glycol dimethyl ether complex (4.33 mg, 0.02 mmol) in 1,2-dimethoxyethane (1.5 mL) was added to the mixture solution in glove box at 20° C. The reaction mixture was stirred under a Lumidox Screen Kit for 4 h at 20° C. The reaction mixture was diluted with water (10 mL) and the result mixture was extracted with ethyl acetate (3×20 mL). The combined organic layers were dried over sodium sulfate and concentrated. The residue was purified by preparative TLC (mobile phase: 12% methanol in ethyl acetate) to give tert-butyl 3-[2-[3-[3-[(4-methyl-1,2,4-triazol-3-yl)methyl]oxetan-3-yl]phenyl]-3-oxo-7-(trifluoromethyl)isoindolin-5-yl]azetidine-1-carboxylate (100 mg, 34.8% yield) as a yellow solid. LCMS [M+H]⁺=584.1.

To a mixture of the intermediate, tert-butyl 3-[2-[3-[3-[(4-methyl-1,2,4-triazol-3-yl)methyl]oxetan-3-yl]phenyl]-3-oxo-7-(trifluoromethyl)isoindolin-5-yl]azetidine-1-carboxylate, (140.0 mg, 0.24 mmol) in dichloromethane (5 mL) was added trifluoroacetic acid (0.3 mL, 0.24 mmol). The mixture was stirred at 18° C. for 3 h and concentrated to dryness under reduced pressure. The residue was purified by RP-HPLC (water (0.2% formic acid)-ACN 8% to 38%) to afford 6-(azetidin-3-yl)-2-(3-(3-((4-methyl-4H-1,2,4-triazol-3-yl)methyl)oxetan-3-yl)phenyl)-4-(trifluoromethyl)isoindolin-1-one (compound 12), (7.2 mg, 5.5% yield) as a yellow solid. ¹H NMR (400 MHz, methanol-d₄): δ 8.19-8.14 (m, 2H), 8.00 (s, 1H), 7.75 (d, J=8.0 Hz, 1H), 7.43-7.38 (m, 2H), 6.79 (d, J=7.6 Hz, 1H), 5.10-5.04 (m, 6H), 4.48 (s, 3H), 4.34 (s, 2H), 3.66 (s, 2H), 2.91 (s, 3H). LCMS [M+H]⁺ or [M−H]⁻: 484.1.

### Example 28: Compound 13

Compound 13 can be synthesized according to Scheme 27, FIG. 9.

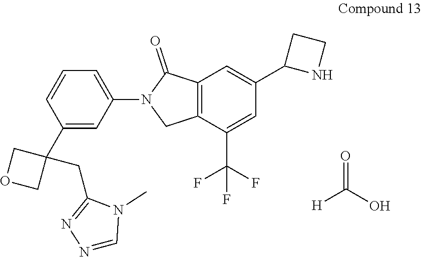 [SMILES: Cn1cnnc1CC1(c2cccc(N3Cc4c(cc(C5CCN5)cc4C(F)(F)F)C3=O)c2)COC1 ; O=CO]

An intermediate, tert-butyl 2-(2-(3-(3-((4-methyl-4H-1,2,4-triazol-3-yl)methyl)oxetan-3-yl)phenyl)-3-oxo-7-(trifluoromethyl)isoindolin-5-yl)azetidine-1-carboxylate, was made as follows. A mixture of 6-bromo-2-(3-(3-((4-methyl-4H-1,2,4-triazol-3-yl)methyl)oxetan-3-yl)phenyl)-4-(trifluoromethyl)isoindolin-1-one (synthesized as in WO2019/148005, 80.0 mg, 0.16 mmol), nickel (II) chloride ethylene glycol dimethyl ether complex (3.5 mg, 0.02 mmol), 1-tert-butoxycarbonylazetidine-2-carboxylic acid (47.6 mg, 0.24 mmol) in N,N-dimethylformamide (3 mL) was added Ir[dF(CF₃)ppy]₂(dtbbpy) PF₆ (1.6 mg, 0.005 mmol), caesium carbonate (154.1 mg, 0.47 mmol) and 4,4′-di-tert-butyl-2,2′-bipyridine (6.35 mg, 0.02 mmol) in glove box at 20° C. The reaction mixture was stirred under a Lumidox Screen Kit for 5 h at 20° C. The reaction mixture was dilute with water (20 mL) and extracted it with ethyl acetate (3×20 mL). The organic layers were combined, dried over sodium sulfate and concentrated to dryness. The residue was purified by preparative TLC (mobile phase: 12% methanol in ethyl acetate) to give tert-butyl 2-(2-(3-(3-((4-methyl-4H-1,2,4-triazol-3-yl)methyl)oxetan-3-yl)phenyl)-3-oxo-7-(trifluoromethyl)isoindolin-5-yl)azetidine-1-carboxylate (60.0 mg, 65.2% yield) as a yellow oil. LCMS [M+H]⁺=584.1.

To a mixture of tert-butyl 3-(2-(3-(3-((4-methyl-4H-1,2,4-triazol-3-yl)methyl)oxetan-3-yl)phenyl)-3-oxo-7-(trifluoromethyl)isoindolin-5-yl)azetidine-1-carboxylate (48.8 mg, 0.08 mmol) in dichloromethane (3 mL) was added trifluoroacetic acid (0.15 mL, 0.08 mmol), the mixture was stirred at 18° C. for 3 h and concentrated to dryness under reduced pressure. The residue was purified by RP-HPLC (water (0.2% formic acid)-ACN 6% to 36%) to afford 6-(azetidin-2-yl)-2-(3-(3-((4-methyl-4H-1,2,4-triazol-3-yl)methyl)oxetan-3-yl)phenyl)-4-(trifluoromethyl)isoindolin-1-one formate (compound 13), (3.04 mg, 6.6% yield) as a yellow solid. ¹H NMR (400 MHz, methanol-d₄): δ 8.46 (br s, 1H), 8.25 (s, 1H), 8.20 (s, 1H), 8.15 (s, 1H), 7.80-7.76 (m, 1H), 7.47-7.38 (m, 2H) 6.82 (d, J=8.0 Hz, 1H), 5.81 (t, J=8.8 Hz, 1H), 5.17 (s, 2H), 5.14-5.07 (m, 4H), 4.31-4.23 (m, 1H), 3.99-3.92 (m, 1H), 3.68 (s, 2H), 3.11-3.05 (m, 1H), 2.94-2.87 (m, 4H). LCMS [M+H]⁺ or [M−H]⁻: 484.1.

### Example 29: Compound 14

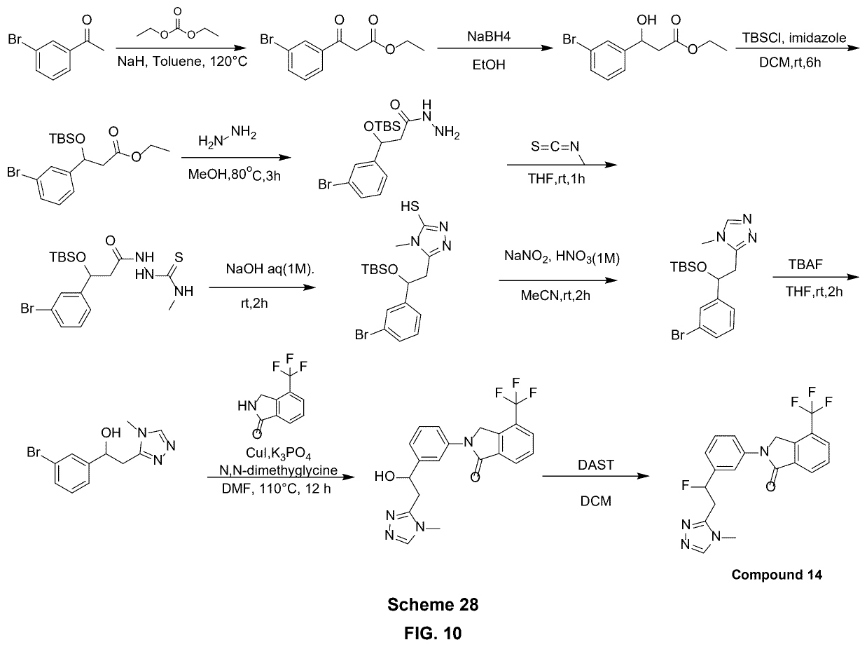

Compound 14 can be synthesized according to Scheme 28, FIG. 10.

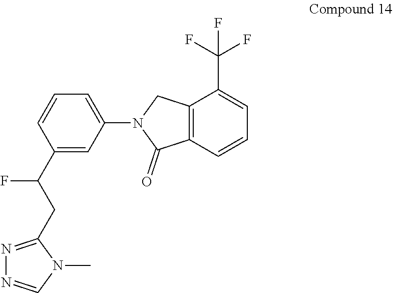 [SMILES: Cn1cnnc1CC(F)c1cccc(N2Cc3c(cccc3C(F)(F)F)C2=O)c1]

A first intermediate, ethyl 3-(3-bromophenyl)-3-oxopropanoate was made as follows. To a solution of 3-bromoacetophenone (15.0 g, 75.36 mmol) in toluene (150 mL) was added sodium hydride (60%, 12.0 g, 301.45 mmol) at 0° C. The mixture was stirred at 25° C. for 2 h and diethyl carbonate (45.7 mL, 376.81 mmol) was added. The reaction mixture was heated at 120° C. for 20 h and cooled. The mixture was quenched with water and adjusted to pH=7 by addition of 1 M HCl solution. The resulting solution was extracted with ethyl acetate (3×500 mL). The combined organic layers were dried over sodium sulfate and concentrated under reduced pressure. The residue was purified by silica gel chromatography (mobile phase: ethyl acetate/petroleum ether, gradient 1% to 2%) to give ethyl 3-(3-bromophenyl)-3-oxo-propanoate (12.0 g, 59% yield) as colorless oil.

A second intermediate, ethyl 3-(3-bromophenyl)-3-hydroxypropanoate, was made as follows. To a solution of 3-(3-bromo-phenyl)-3-oxo-propionic acid ethyl ester (8.50 g, 31.35 mmol) in ethanol (100 mL) was added sodium borohydride (3.60 g, 94.06 mmol) at 25° C. The mixture was stirred at 25° C. for 2 h and quenched by addition of water (200 mL). The solution was extracted with ethyl acetate (3×200 mL). The combined organic layers were washed with brine (100 mL), dried and concentrated under reduced pressure. The residue was purified by silica gel chromatography (mobile phase: ethyl acetate/petroleum ether, gradient 0% to 3%) to afford ethyl 3-(3-bromophenyl)-3-hydroxy-propanoate (4.00 g, 47% yield) as a colorless oil.

A third intermediate, ethyl 3-(3-bromophenyl)-3-((tert-butyldimethylsilyl)oxy)-propanoate, was made as follows. To a solution of [ethyl 3-(3-bromophenyl)-3-hydroxy-propanoate (4.00 g, 14.65 mmol) in dichloromethane (70 mL) was added imidazole (1.50 g, 21.97 mmol) and tert-butyldimethylchlorosilane (2.90 g, 19.04 mmol). The resulting solution was stirred at 20° C. for 2 h and diluted with water (200 mL). The mixture was then extracted with dichloromethane (3×200 mL). The combined organic layers were dried over anhydrous sodium sulfate and concentrated under reduced pressure. The residue was purified by silica gel chromatography (mobile phase: ethyl acetate/petroleum ether, gradient 0% to 2.5%) to afford ethyl 3-(3-bromophenyl)-3-[tert-butyl(dimethyl)silyl]oxy-propanoate (4.20 g, 74% yield)] as a colorless oil.

A fourth intermediate, 3-(3-bromophenyl)-3-((tert-butyldimethylsilyl)oxy)propane-hydrazide, was made as follows. To a solution of ethyl 3-(3-bromophenyl)-3-[tert-butyl(dimethyl)silyl]oxy-propanoate (4.20 g, 10.84 mmol) in methanol (50 mL) was added hydrazine monohydrate (9.9 mL, 171.88 mmol). The mixture was heated at 80° C. for 12 h and concentrated under reduced pressure to afford crude 3-(3-bromophenyl)-3-[tert-butyl-(dimethyl)silyl]oxy-propanehydrazide (4.00 g, 98% yield) as a white solid. LCMS [M+H]⁺=375.0.

A fifth intermediate, 2-(3-(3-bromophenyl)-3-((tert-butyldimethylsilyl)oxy)propan-oyl)-N-methylhydrazinecarbothioamide was made as follows. To a mixture of 3-(3-bromophenyl)-3-[tert-butyl(dimethyl)silyl]oxy-propanehydrazide (4.00 g, 10.71 mmol) in tetrahydrofuran (75 mL) was added methyl isothiocyanate (1.60 g, 21.43 mmol) at 25° C. The mixture was stirred at 25° C. for 4 h and concentrated under reduced pressure to afford crude 1-[[3-(3-bromophenyl)-3-[tert-butyl(dimethyl)silyl]oxy-propanoyl]amino]-3-methyl-thiourea (4.00 g, 84% yield) as white solid. LCMS [M+H]+=448.0.

A sixth intermediate, 5-(2-(3-bromophenyl)-2-((tert-butyldimethylsilyl)oxy)ethyl)-4-methyl-4H-1,2,4-triazole-3-thiol, was made as follows. A mixture of 1-[[3-(3-bromophenyl)-3-[tert-butyl(dimethyl)silyl]oxy-propanoyl]amino]-3-methyl-thiourea (4.0 g, 8.96 mmol) in 1 M sodium hydroxide (42.0 mL, 42.0 mmol) was stirred at 25° C. for 1 h and adjusted to pH=5 by addition of 1 M HCl. The formed solid was collected by filtration and dried to afford crude 5-[2-(3-bromophenyl)-2-[tert-butyl(dimethyl)silyl]oxy-ethyl]-4-methyl-4H-1,2,4-triazole-3-thiol (3.6 g, 95% yield) as a yellow solid. LCMS [M+H]⁺=428.0.

A seventh intermediate, 3-(2-(3-bromophenyl)-2-((tert-butyldimethylsilyl)oxy)-ethyl)-4-methyl-4H-1,2,4-triazole, was made as follows. To a solution of 5-[2-(3-bromophenyl)-2-[tert-butyl(dimethyl)silyl]oxy-ethyl]-4-methyl-1,2,4-triazole-3-thiol (3.60 g, 8.52 mmol) in water (9 mL) and acetonitrile (9 mL) was added sodium nitrite (5.90 g, 85.19 mmol), followed by addition of 1 M nitric acid (41.0 mL, 41.00 mmol) dropwise at 0° C. After addition, the mixture was stirred for another 1 h at 20° C. and quenched by addition of saturated aqueous NaHCO₃ (100 mL). The resulting mixture was extracted with ethyl acetate (3×100 mL). The combined organic layers were washed with brine (100 mL), dried and concentrated under reduced pressure. The residue was purified by silica gel chromatography (mobile phase: methanol/dichloromethane, gradient 0% to 5%) to afford 3-(2-(3-bromophenyl)-2-((tert-butyldimethylsilyl)oxy)ethyl)-4-methyl-4H-1,2,4-triazole (3.3 g, 99% yield) as yellow solid. LCMS [M+H]⁺=398.1.

An eighth intermediate, 1-(3-bromophenyl)-2-(4-methyl-4H-1,2,4-triazol-3-yl)ethanol, was made as follows. To a mixture of [1-(3-bromophenyl)-2-(4-methyl-1,2,4-triazol-3-yl)ethoxy]-tert-butyl-dimethyl-silane (3.3 g, 8.45 mmol) in tetrahydrofuran (80 mL) was added tetrabutylammonium fluoride (1 M in tetrahydrofuran, 12.68 mL, 12.68 mmol) at 20° C. The mixture was stirred for 1 h and diluted with water (100 mL). The resulting mixture was extracted with ethyl acetate (3×100 mL). The combined organic layers were washed with brine (2×55 mL), dried over sodium sulfate and concentrated under reduced pressure. The residue was purified by silica gel chromatography (mobile phase: ethyl acetate/petroleum ether, gradient 0% to 100%) to afford 1-(3-bromophenyl)-2-(4-methyl-4H-1,2,4-triazol-3-yl)ethanol (2.30 g, 96% yield) as a yellow solid. LCMS [M+H]⁺=282.1.

A ninth intermediate, 2-(3-(1-hydroxy-2-(4-methyl-4H-1,2,4-triazol-3-yl)ethyl)-phenyl)-4-(trifluoromethyl)isoindolin-1-one, was made as follows. A mixture of cesium carbonate (346.5 mg, 1.06 mmol), 1-(3-bromophenyl)-2-(4-methyl-1,2,4-triazol-3-yl)ethanol (100.0 mg, 0.35 mmol), copper(I) iodide (13.5 mg, 0.07 mmol), 4-(trifluoromethyl)isoindolin-1-one (89.1 mg, 0.44 mmol) and N¹,N²-dimethylethane-1,2-diamine (6.3 mg, 0.07 mmol) in 1,4-dioxane (5 mL) was heated at 110° C. for 12 h under nitrogen protection. After cooled, the reaction was diluted with water (15 mL) and extracted with ethyl acetate (3×15 mL). The combined organic layers were washed with brine (2×15 mL), dried over sodium sulfate and concentrated under reduced pressure. The residue was purified by preparative TLC (solvent gradient: 10% methanol in dichloromethane) to afford 2-[3-[1-hydroxy-2-(4-methyl-4H-1,2,4-triazol-3-yl)ethyl]phenyl]-4-(trifluoromethyl)isoindolin-1-one (28.0 mg, 20% yield) as a brown solid. LCMS [M+H]⁺=403.1.

To a mixture of the ninth intermediate, 2-[3-[1-hydroxy-2-(4-methyl-1,2,4-triazol-3-yl)ethyl]phenyl]-4-(trifluoromethyl)isoindolin-1-one, (27.0 mg, 0.07 mmol) in dichloromethane (1 mL) was added DAST (16.2 mg, 0.10 mmol) at 18° C. The mixture was stirred for 1 h and quenched by addition of saturated aqueous NaHCO₃ (5 mL). The solution was extracted with dichloromethane (3×5 mL). The combined organic layers were dried over sodium sulfate and concentrated under reduced pressure. The residue was purified by RP-HPLC (water (0.05% NH₃HCO₃+10 mM NH₄HCO₃)-ACN 27% to 57%) to afford 2-[3-[1-fluoro-2-(4-methyl-4H-1,2,4-triazol-3-yl)ethyl]phenyl]-4-(trifluoromethyl)isoindolin-1-one (Compound 14) (2.8 mg, 10% yield) as a light yellow solid. ¹H NMR (400 MHz, methanol-d₄): δ 8.34 (s, 1H), 8.11 (d, J=8.0 Hz, 1H), 8.00-7.90 (m, 3H), 7.80-7.78 (m, 1H), 7.53-7.49 (m, 1H), 7.28-7.26 (m, 1H), 6.03-5.88 (m, 1H), 5.20 (s, 2H), 3.63 (s, 3H), 3.58-3.44 (m, 2H). LCMS [M+H]⁺ or [M−H]⁻: 405.1.

### Example 30: Compound 15

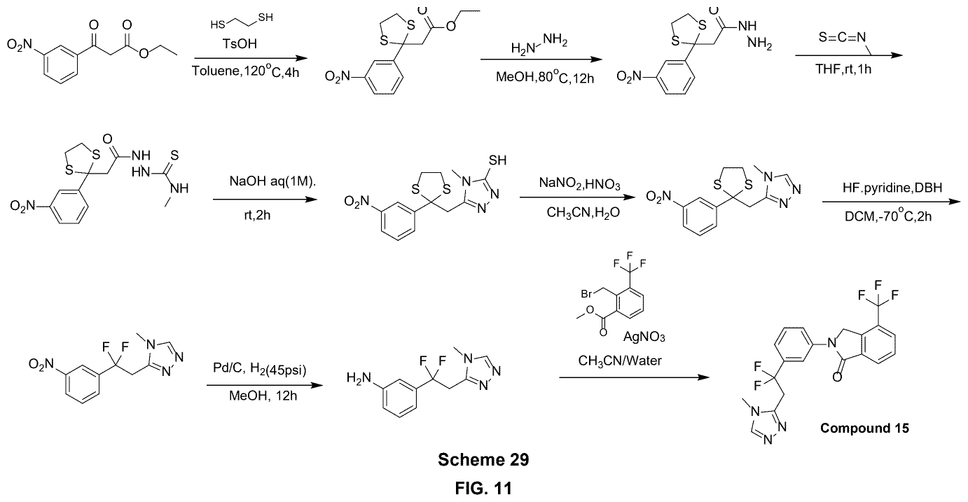

Compound 15 can be synthesized according to Scheme 29, FIG. 11.

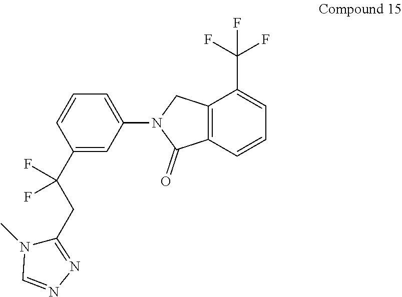 [SMILES: Cn1cnnc1CC(F)(F)c1cccc(N2Cc3c(cccc3C(F)(F)F)C2=O)c1]

A first intermediate, ethyl 2-(2-(3-nitrophenyl)-1,3-dithiolan-2-yl)acetate, was made as follows. A solution of ethyl 3-(3-nitrophenyl)-3-oxopropanoate (2.0 g, 8.43 mmol), ethane-1,2-dithiol (2.8 g, 30.15 mmol) and p-toluenesulfonic acid (290.4 mg, 1.69 mmol) in toluene (40.0 mL) was stirred at 120° C. for 3 hours. The mixture was concentrated and the residue was purified by silica gel chromatography (mobile phase: ethyl acetate/petroleum ether, gradient 0% to 2.5%) to afford ethyl 2-[2-(3-nitrophenyl)-1,3-dithiolan-2-yl]acetate (1.0 g, 37.8% yield) as light yellow oil. ¹H NMR (400 MHz, CDCl₃): δ 8.64 (s, 1H), 8.11-8.08 (m, 2H), 7.49 (t, J=8 Hz, 1H), 4.02-4.00 (m, 2H), 3.55 (s, 2H), 3.45-3.32 (m, 4H), 1.14 (t, J=7.2 Hz, 3H).

A second intermediate, 2-(2-(3-nitrophenyl)-1,3-dithiolan-2-yl)acetohydrazide, was made as follows. To a solution of ethyl 2-[2-(3-nitrophenyl)-1,3-dithiolan-2-yl]acetate (1.0 g, 3.19 mmol) in methanol (20.0 mL) was added hydrazine monohydrate (3.0 mL, 52.0 mmol), then the reaction mixture was heated at 80° C. for 12 h. The mixture was concentrated to give crude 2-[2-(3-nitrophenyl)-1,3-dithiolan-2-yl]acetohydrazide (900.0 mg, 94.2% yield) as a yellow solid. LCMS [M+H]⁺=300.1.

A third intermediate, N-methyl-2-(2-(2-(3-nitrophenyl)-1,3-dithiolan-2-yl)acetyl)-hydrazine-1-carbothioamide, was made as follows. To a mixture of 2-[2-(3-nitrophenyl)-1,3-dithiolan-2-yl]acetohydrazide (900.0 mg, 3.01 mmol) in tetrahydrofuran (25.0 mL) was added methyl isothiocyanate (439.6 mg, 6.01 mmol) at 25° C. and stirred for 4 hours. The mixture was concentrated to afford crude N-methyl-2-(2-(2-(3-nitrophenyl)-1,3-dithiolan-2-yl)acetyl)hydrazinecarbothioamide (1.0 g, 89.3% yield) as yellow solid. LCMS [M+H]⁺=373.0.

A fourth intermediate, 4-methyl-5-((2-(3-nitrophenyl)-1,3-dithiolan-2-yl)methyl)-4H-1,2,4-triazole-3-thiol, was made as follows. A mixture of N-methyl-2-(2-(2-(3-nitrophenyl)-1,3-dithiolan-2-yl)acetyl)hydrazinecarbothioamide (1.0 g, 2.68 mmol) in 1 M sodium hydroxide (12.0 mL, 12 mmol) was stirred at 25° C. for 1 h and diluted with water (40.0 mL) then adjusted to pH=5 by addition of 1 M HCl solution. The formed solid was collected by filtration and dried to afford crude 4-methyl-5-((2-(3-nitrophenyl)-1,3-dithiolan-2-yl)methyl)-4H-1,2,4-triazole-3-thiol (760.0 mg, 79.9% yield) as a yellow solid. LCMS [M+H]⁺=355.0.

A fifth intermediate, 4-methyl-3-((2-(3-nitrophenyl)-1,3-dithiolan-2-yl)methyl)-4H-1,2,4-triazole, was made as follows. To a solution of 4-methyl-5-((2-(3-nitrophenyl)-1,3-dithiolan-2-yl)methyl)-4H-1,2,4-triazole-3-thiol (740.0 mg, 2.09 mmol) in water (2.0 mL) and acetonitrile (4.0 mL) was added sodium nitrite (1.40 g, 20.88 mmol), followed by addition of 1 M nitric acid (20.9 mL, 20.90 mmol) dropwise at 0° C. After addition, the mixture was stirred for another 1 h at 20° C. and quenched by addition of saturated aqueous NaHCO₃ (20 mL). The resulting mixture was extracted with ethyl acetate (3×80 mL). The combined organic layers were washed with brine (50 mL), dried and concentrated under reduced pressure. The residue was purified by silica gel chromatography (mobile phase: methanol/dichloromethane, gradient 0% to 5%) to afford 4-methyl-3-((2-(3-nitrophenyl)-1,3-dithiolan-2-yl)methyl)-4H-1,2,4-triazole (430.0 mg, 63.9% yield) as yellow solid. ¹H NMR (400 MHz, CDCl₃): δ 8.63-8.61 (m, 1H), 8.14-8.11 (m, 1H), 8.04-7.99 (m, 1H), 7.48 (t, J=8.0 Hz, 1H), 3.83 (s, 2H), 3.49 (s, 3H), 3.41-3.38 (m, 2H), 3.32-3.29 (m, 2H).

A sixth intermediate, 3-(2,2-difluoro-2-(3-nitrophenyl)ethyl)-4-methyl-4H-1,2,4-triazole, was made as follows. A solution of 1,3-dibromo-5,5-dimethyl-2,4-imidazolidinedione (532.1 mg, 1.86 mmol) in anhydrous dichloromethane (5.0 mL) was cooled to −70° C. Pyridine hydrofluoride (0.48 mL, 3.72 mmol, 70% HF) was added dropwise at a temperature below −65° C., and the mixture stirred at −70° C. for 30 min. A solution of 4-methyl-3-((2-(3-nitrophenyl)-1,3-dithiolan-2-yl)methyl)-4H-1,2,4-triazole (150.0 mg, 0.47 mmol) in dichloromethane (1.0 mL) was added dropwise and the mixture stirred at −70° C. for 1 h. The mixture was poured into 2M sodium hydroxide (10.0 mL) containing 39% NaHSO3 (10.0 mL) solution. The aqueous layer was extracted with dichloromethane (2×10 mL) Combined the organic phase and washed with brine (10 mL), dried over sodium sulfate, filtered and the filtrate was concentrated under reduced pressure. The residue was purified by chromatography on silica (mobile phase: methanol/dichloromethane, gradient 0% to 5%) to give 3-(2,2-difluoro-2-(3-nitrophenyl)ethyl)-4-methyl-4H-1,2,4-triazole (70 mg, 56.1% yield) as yellow solid. LCMS [M+H]⁺=269.1.

A seventh intermediate, 3-(1,1-difluoro-2-(4-methyl-4H-1,2,4-triazol-3-yl)ethyl)aniline, was made as follows. To a mixture of 3-(2,2-difluoro-2-(3-nitrophenyl)ethyl)-4-methyl-4H-1,2,4-triazole (70.0 mg, 0.26 mmol) in methanol (3.0 mL) was added and Pd/C (27.8 mg, 0.03 mmol)(10% on carbon), the mixture was stirred at 20° C. for 12 hours under H2 (45 psi). The mixture was filtered and the filtrate was concentrated under reduced pressure to give 3-(1,1-difluoro-2-(4-methyl-4H-1,2,4-triazol-3-yl)ethyl)aniline (50.0 mg, 80.4% yield) as yellow solid. LCMS [M+H]⁺=239.1.

Finally, to a mixture of methyl 2-(bromomethyl)-3-(trifluoromethyl)benzoate (WO 2019148005, 62.4 mg, 0.21 mmol) and 3-(1,1-difluoro-2-(4-methyl-4H-1,2,4-triazol-3-yl)ethyl)aniline (50.0 mg, 0.21 mmol) in acetonitrile (2.2 mL) and water (0.7 mL) was added a solution of silver nitrate (53.5 mg, 0.31 mmol) at 0° C., the mixture was stirred at 25° C. for 16 h and filtered. The filtrate was concentrated under reduced pressure. The residue was purified by RP-HPLC (water (0.05% NH₃H₂O+10 mM NH₄HCO₃)-ACN 40% to 70%) to afford 2-(3-(1,1-difluoro-2-(4-methyl-4H-1,2,4-triazol-3-yl)ethyl)phenyl)-4-(trifluoromethyl)isoindolin-1-one (Compound 15) (15.68 mg, 17.5% yield) as a yellow oil. ¹H NMR (400 MHz, methanol-d₄): δ 8.43 (s, 1H), 8.13-8.09 (m, 2H), 7.99 (d, J=7.6 Hz, 2H), 7.79 (d, J=7.6 Hz, 1H), 7.56 (t, J=8 Hz, 1H), 7.34 (d, J=8.0 Hz, 1H), 5.21 (s, 2H) 3.89 (t, J=15.2 Hz, 2H), 3.64 (s, 3H). LCMS [M+H]⁺ or [M−H]⁻: 423.1.

### Example 31: Compound 17

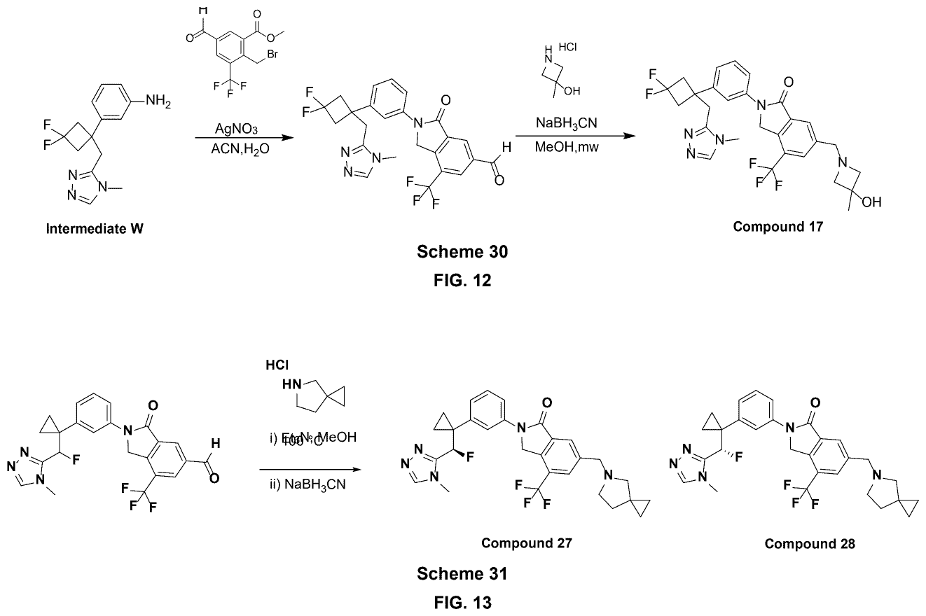

Compound 17, 2-(3-(3,3-difluoro-1-((4-methyl-4H-1,2,4-triazol-3-yl)methyl)cyclo-butyl)phenyl)-6-((3-hydroxy-3-methylazetidin-1-yl)methyl)-4-(trifluoromethyl)isoindolin-1-one, can be synthesized according to Scheme 30, FIG. 12.

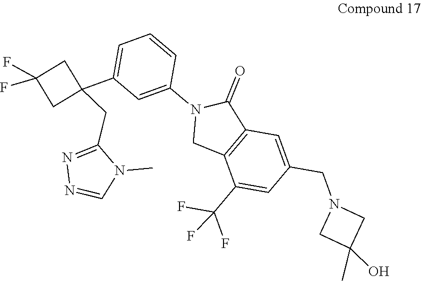 [SMILES: Cn1cnnc1CC1(c2cccc(N3Cc4c(cc(CN5CC(C)(O)C5)cc4C(F)(F)F)C3=O)c2)CC(F)(F)C1]

An intermediate, 2-(3-(3,3-difluoro-1-((4-methyl-4H-1,2,4-triazol-3-yl)methyl)cyclobutyl)phenyl)-3-oxo-7-(trifluoromethyl)isoindoline-5-carbaldehyde, was made as follows. To a mixture of methyl 2-(bromomethyl)-5-formyl-3-(trifluoromethyl)benzoate (synthesized as in WO 2019/148005; 385.5 mg, 1.19 mmol) and 3-(3,3-difluoro-1-((4-methyl-4H-1,2,4-triazol-3-yl)methyl)cyclobutyl)aniline (330.0 mg, 1.19 mmol) in acetonitrile (6 mL) and water (3 mL) was added a solution of silver nitrate (241.7 mg, 1.42 mmol) in water (0.2 mL) at 0° C., the mixture was stirred at 25° C. for 16 h and filtered. The filtrate was concentrated under reduced pressure. The residue was purified by silica gel chromatography (mobile phase: methanol/dichloromethane, gradient 0% to 10%) to afford 2-(3-(3,3-difluoro-1-((4-methyl-4H-1,2,4-triazol-3-yl)methyl)cyclobutyl)phenyl)-3-oxo-7-(trifluoromethyl)isoindoline-5-carbaldehyde (500.0 mg, 86% yield) as a yellow oil. LCMS [M+H]⁺=491.2.

To a solution of 3-methylazetidin-3-ol hydrochloride (50.4 mg, 0.41 mmol) and triethylamine (41.3 mg, 0.41 mmol) in methanol (5 mL) was added the purified intermediate, 2-(3-(3,3-difluoro-1-((4-methyl-4H-1,2,4-triazol-3-yl)methyl)cyclobutyl)phenyl)-3-oxo-7-(trifluoromethyl)isoindoline-5-carbaldehyde (50.0 mg, 0.10 mmol). The mixture was heated under microwaves irrigation at 100° C. for 1 minute and cooled down to room temperature. The mixture was then added sodium cyanoborohydride (11.5 mg, 0.18 mmol) and heated at 80° C. under microwave irrigation for another 45 minutes. The reaction was quenched with 1 M HCl solution and the result solution was adjusted to pH=7 by addition of saturated NaHCO₃ solution. The mixture was extracted with dichloromethane (3×10 mL). The combined organic phases were dried over anhydrous sodium sulphate and concentrated under reduced pressure. The residue was purified by RP-HPLC (acetonitrile 22-52%/0.05% NH₄OH in water) to afford 2-(3-(3,3-difluoro-1-((4-methyl-4H-1,2,4-triazol-3-yl)methyl)cyclo-butyl)phenyl)-6-((3-hydroxy-3-methylazetidin-1-yl)methyl)-4-(trifluoromethyl)isoindolin-1-one (Compound 17) (13.2 mg, 22% yield) as a white solid, characterized as follows. ¹H NMR (400 MHz, methanol-d₄): δ 8.18 (s, 1H), 8.03 (s, 1H), 7.92 (s, 1H), 7.76-7.73 (m, 1H), 7.48 (s, 1H), 7.40 (t, J=8.0 Hz, 1H), 6.84 (d, J=7.6 Hz, 1H), 5.09 (s, 2H), 3.87 (s, 2H), 3.37-3.34 (m, 4H), 3.31-3.24 (m, 2H), 3.15 (d, J=8.4 Hz, 2H), 3.06-3.02 (m, 2H), 2.78 (s, 3H), 1.49 (s, 3H). LCMS [M+H]⁺ or [M−H]⁻: 562.1.

### Example 32: Compound 27 and Compound 28

Compounds 27 and 28 can be synthesized according to Scheme 31, FIG. 13.

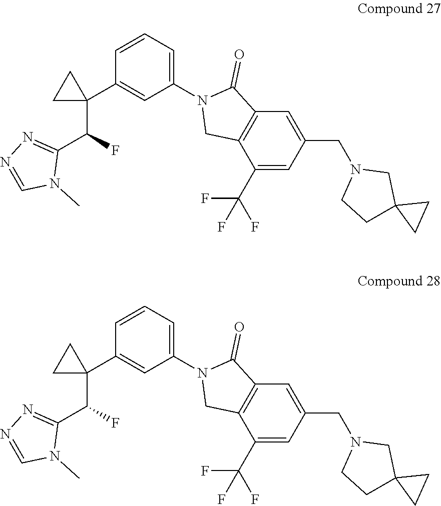 [SMILES: Cn1cnnc1[C@H](F)C1(c2cccc(N3Cc4c(cc(CN5CCC6(CC6)C5)cc4C(F)(F)F)C3=O)c2)CC1 ; Cn1cnnc1[C@@H](F)C1(c2cccc(N3Cc4c(cc(CN5CCC6(CC6)C5)cc4C(F)(F)F)C3=O)c2)CC1]

A microwave vial was charged with 2-(3-(1-(fluoro(4-methyl-4H-1,2,4-triazol-3-yl)methyl)cyclopropyl)phenyl)-3-oxo-7-(trifluoromethyl)isoindoline-5-carbaldehyde (Intermediate K, Scheme 11, 80 mg, 0.175 mmol) and 5-azaspiro[2.4]heptane hydrochloride (70 mg, 0.524 mmol) in methanol (1.75 mL). Triethylamine (71 μL, 0.506 mmol) was added and the reaction was heated in a microwave oven for 1 min at 100° C. The reaction was cooled to RT and placed under nitrogen before sodium cyanoborohydride (45 mg, 0.716 mmol) was added. The reaction was heated in a microwave oven at 100° C. for 30 minutes. A few drops of HCl 1 N was added and the reaction mixture was basified with saturated sodium bicarbonate (2 mL). The product was extracted with 40% iPrOH in CHCl₃ (3×2 mL). The organic layers were combined, dried over sodium sulfate, and concentrated. The residue was purified by chromatography on C18 silica gel (0-100% acetonitrile in ammonium formate, pH=4). After chiral separation (SFC: Column: Lux Cel-4, 10×250 mm 5 um, Mode: Isocratic, Mobile phase: 60% IPA, 40% supercritical CO₂, Flow rate: 10 mL/min, Backpressure: 150 bar, Column Temperature: 40° C., Run time (min): 10), desired products were obtained and characterized as follows.

(R)-6-(5-azaspiro[2.4]heptan-5-ylmethyl)-2-(3-(1-(fluoro(4-methyl-4H-1,2,4-triazol-3-yl)methyl)cyclopropyl)phenyl)-4-(trifluoromethyl)isoindolin-1-one (12 mg, 58% yield) (27). ¹H NMR (400 MHz, DMSO-d₆) δ 8.36 (s, 1H), 7.96 (d, J=19.0 Hz, 2H), 7.87-7.70 (m, 2H), 7.35 (t, J=7.9 Hz, 1H), 7.10 (d, J=7.8 Hz, 1H), 5.78 (d, J=44.5 Hz, 1H), 5.11 (q, J=17.2 Hz, 2H), 3.80 (s, 2H), 3.22 (s, 3H), 2.70 (t, J=6.8 Hz, 2H), 2.45 (s, 2H), 1.77 (t, J=6.8 Hz, 2H), 1.28-1.16 (m, 2H), 1.12-0.96 (m, 2H), 0.50 (d, J=3.8 Hz, 4H). LCMS [M+H]⁺ or [M−H]⁻: 540.3.

(S)-6-(5-azaspiro[2.4]heptan-5-ylmethyl)-2-(3-(1-(fluoro(4-methyl-4H-1,2,4-triazol-3-yl)methyl)cyclopropyl)phenyl)-4-(trifluoromethyl)isoindolin-1-one (12 mg, 58% yield). (28) 1H NMR (400 MHz, DMSO-d₆) δ 8.36 (s, 1H), 7.97 (d, J=19.3 Hz, 2H), 7.84 (d, J=9.6 Hz, 1H), 7.75 (s, 1H), 7.35 (t, J=7.9 Hz, 1H), 7.10 (d, J=7.7 Hz, 1H), 5.79 (d, J=44.5 Hz, 1H), 5.22-5.02 (m, 2H), 3.80 (s, 2H), 3.22 (s, 1H), 2.70 (t, J=6.8 Hz, 2H), 2.45 (s, 2H), 1.77 (t, J=6.9 Hz, 2H), 1.23 (s, 2H), 1.10-0.94 (m, 2H), 0.50 (d, J=3.8 Hz, 4H). LCMS [M+H]⁺ or [M−H]⁻: 540.3.

### Example 33: Compound 29 and Compound 30

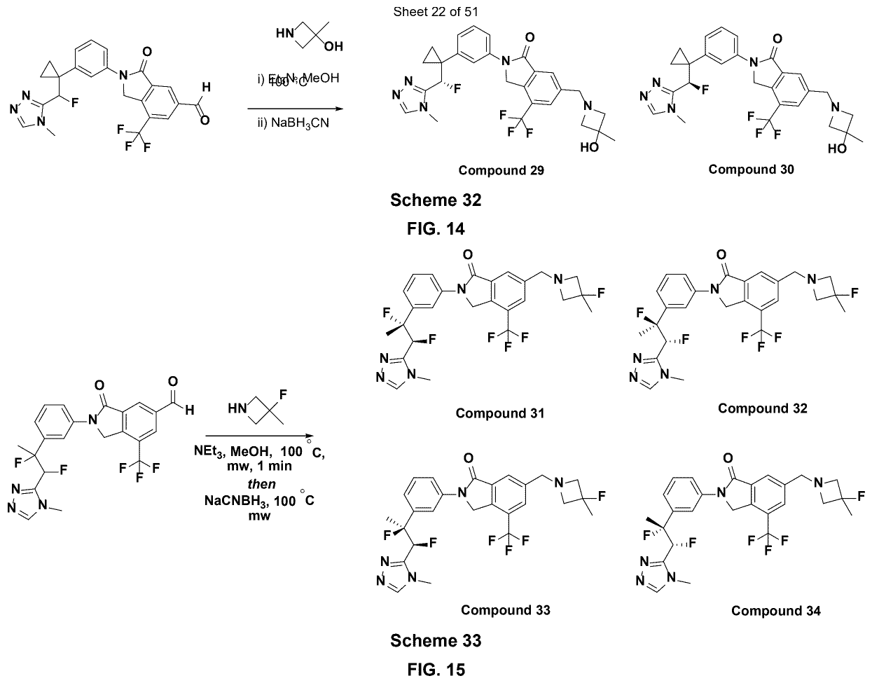

Compounds 29 and 30 can be synthesized according to Scheme 32, FIG. 14.

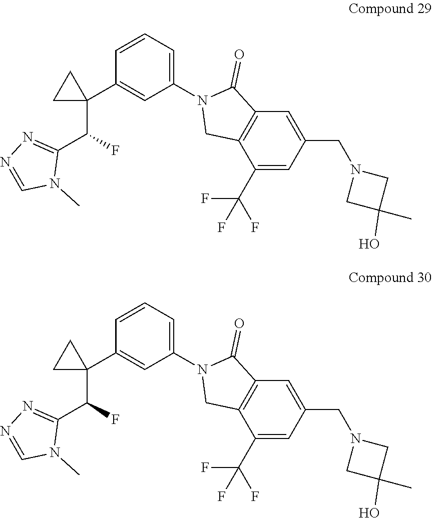 [SMILES: Cn1cnnc1[C@@H](F)C1(c2cccc(N3Cc4c(cc(CN5CC(C)(O)C5)cc4C(F)(F)F)C3=O)c2)CC1 ; Cn1cnnc1[C@H](F)C1(c2cccc(N3Cc4c(cc(CN5CC(C)(O)C5)cc4C(F)(F)F)C3=O)c2)CC1]

Similar chemistry to that of Example 37 was used to prepare Compounds 29 and 30. After chiral separation (SFC: Column: Lux Cel-4, 10×250 mm 5 um. Mode: Isocratic.Mobile phase: 60% MeOH-0.1% NH₄OH, 40% supercritical CO₂. Flow rate: 10 mL/min. Backpressure: 150 bar. Column Temperature: 40° C. Run time: 7 min, RT1=3.81 min, RT_{2=5.66} min), desired products were obtained and characterized as follows.

(S)-2-(3-(1-(fluoro(4-methyl-4H-1,2,4-triazol-3-yl)methyl)cyclopropyl)phenyl)-6-((3-hydroxy-3-methylazetidin-1-yl)methyl)-4-(trifluoromethyl)isoindolin-1-one (29) (14 mg, 12% yield). ¹H NMR (400 MHz, DMSO-d₆) δ 8.36 (s, 1H), 7.98-7.71 (m, 4H), 7.34 (t, J=8.0 Hz, 1H), 7.09 (d, J=7.8 Hz, 1H), 5.78 (d, J=44.5 Hz, 1H), 5.19-5.03 (m, 2H), 3.79 (s, 2H), 3.23-3.17 (m, 5H), 2.96 (d, J=7.5 Hz, 2H), 1.38 (s, 3H), 1.27-1.18 (m, 2H), 1.12-0.99 (m, 2H). LCMS [M+H]⁺ or [M−H]⁻: 530.3.

(R)-2-(3-(1-(fluoro(4-methyl-4H-1,2,4-triazol-3-yl)methyl)cyclopropyl)phenyl)-6-((3-hydroxy-3-methylazetidin-1-yl)methyl)-4-(trifluoromethyl)isoindolin-1-one (22 mg, 19% yield). (30)¹H NMR (400 MHz, DMSO-d₆) δ 8.36 (s, 1H), 7.97-7.68 (m, 4H), 7.34 (t, J=7.9 Hz, 1H), 7.09 (d, J=7.7 Hz, 1H), 5.78 (d, J=44.5 Hz, 1H), 5.18-5.00 (m, 2H), 3.79 (s, 2H), 3.24-3.17 (m, 5H), 2.96 (d, J=7.5 Hz, 2H), 1.38 (s, 3H), 1.28-1.10 (m, 2H), 1.10-1.01 (m, 2H). LCMS [M+H]⁺ or [M−H]⁻: 530.3.

### Example 34: Compound 31, Compound 32, Compound 33 and Compound 34

Compounds 31, 32, 33, and 34, stereoisomers, can be synthesized according to Scheme 33, FIG. 15.

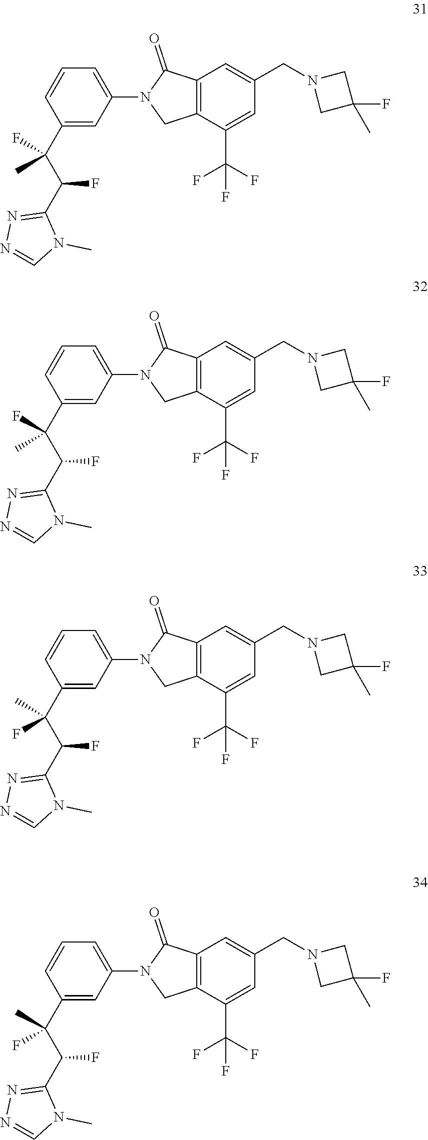 [SMILES: Cn1cnnc1[C@H](F)[C@](C)(F)c1cccc(N2Cc3c(cc(CN4CC(C)(F)C4)cc3C(F)(F)F)C2=O)c1 ; Cn1cnnc1[C@@H](F)[C@@](C)(F)c1cccc(N2Cc3c(cc(CN4CC(C)(F)C4)cc3C(F)(F)F)C2=O)c1 ; Cn1cnnc1[C@H](F)[C@@](C)(F)c1cccc(N2Cc3c(cc(CN4CC(C)(F)C4)cc3C(F)(F)F)C2=O)c1 ; Cn1cnnc1[C@@H](F)[C@](C)(F)c1cccc(N2Cc3c(cc(CN4CC(C)(F)C4)cc3C(F)(F)F)C2=O)c1]

A microwave vial was charged with 2-[3-[1,2-difluoro-1-methyl-2-(4-methyl-1,2,4-triazol-3-yl)ethyl]phenyl]-3-oxo-7-(trifluoromethyl)isoindoline-5-carbaldehyde (Intermediate J, Scheme 10, FIG. 6J, 67.0 mg, 0.14 mmol) and 3-fluoro-3-methyl-azetidine hydrochloride (91.7 mg, 0.74 mmol) in methanol (1.44 mL). Triethylamine (0.10 mL, 0.71 mmol) was added and the reaction was heated in a microwave oven for 1 min at 100° C. The reaction was cooled to rt and placed under nitrogen before sodium cyano borohydride (16.3 mg, 0.26 mmol) was added. The reaction was heated in a microwave oven at 100° C. for 30 minutes. A few drops of HCl 1 N was added and the reaction mixture was basified with saturated NaHCO₃ (2 mL). The product was extracted with 40% iPrOH in CHCl₃ (3×2 mL). The organic layers were combined, dried over sodium sulfate, and concentrated. The residue was purified by chromatography on C18 silica gel (0-100% acetonitrile in ammonium formate, pH=4). After chiral separation [First pass: Anal. Column: ChiralPak IB, 250 mm×4.6 mm ID, 5 μm. Mobile Phase: 10:10:80 MeOH:EtOH:Hexane. Isocratic Flow: 0.8 mL/min, (pressure was 44.5 bars). Column Temp.: ˜26° C. Run Time: 20 min.] Second pass: [Anal. Column: ChiralPak IA, 250 mm×4.6 mm ID, 5 μm. Mobile Phase: 15:15:70 MeOH:EtOH:Hexane. Isocratic Flow: 0.8 mL/min, (pressure was 57 bars). Column Temp.: ˜26° C. Run Time: 25 min.], desired products were obtained.

2-(3-((1 S,2R)-1,2-difluoro-1-(4-methyl-4H-1,2,4-triazol-3-yl)propan-2-yl)phenyl)-6-((3-fluoro-3-methylazetidin-1-yl)methyl)-4-(trifluoromethyl)isoindolin-1-one (4.9 mg, 6.3% yield). (31)¹H NMR (400 MHz, DMSO-d₆) δ 8.43 (s, 1H), 8.06-7.77 (m, 4H), 7.43 (t, J=8.0 Hz, 1H), 7.16 (d, J=7.7 Hz, 1H), 6.33 (dd, J=42.5, 22.7 Hz, 1H), 5.21-5.08 (m, 2H), 3.86 (s, 2H), 3.53 (s, 3H), 1.95 (d, J=23.5 Hz, 3H), 1.55 (d, J=22.4 Hz, 3H). LCMS [M+H]⁺ or [M−H]⁻: 538.0.

2-(3-((1 R,2S)-1,2-difluoro-1-(4-methyl-4H-1,2,4-triazol-3-yl)propan-2-yl)phenyl)-6-((3-fluoro-3-methylazetidin-1-yl)methyl)-4-(trifluoromethyl)isoindolin-1-one (6.9 mg, 8.9% yield). (32)¹H NMR (400 MHz, DMSO-d₆) δ 8.43 (s, 1H), 8.02-7.82 (m, 4H), 7.43 (t, J=8.0 Hz, 1H), 7.16 (d, J=8.3 Hz, 1H), 6.33 (dd, J=42.4, 22.7 Hz, 1H), 5.20-5.04 (m, 2H), 3.86 (s, 2H), 3.53 (s, 3H), 3.38-3.34 (m, 1H), 3.27-3.23 (m, 1H), 1.95 (dd, J=23.5, 2.0 Hz, 3H), 1.55 (d, J=22.4 Hz, 3H). LCMS [M+H]⁺ or [M−H]⁻: 538.0.

2-(3-((1 S,2S)-1,2-difluoro-1-(4-methyl-4H-1,2,4-triazol-3-yl)propan-2-yl)phenyl)-6-((3-fluoro-3-methylazetidin-1-yl)methyl)-4-(trifluoromethyl)isoindolin-1-one (4.3 mg, 5.5% yield). (33)¹H NMR (400 MHz, DMSO-d₆) δ 8.51 (s, 1H), 8.06-7.85 (m, 4H), 7.48 (t, J=8.3 Hz, 1H), 7.27 (d, J=7.9 Hz, 1H), 6.41 (dd, J=43.5, 21.6 Hz, 1H), 5.35-5.10 (m, 2H), 3.87 (s, 2H), 3.50 (s, 3H), 1.82 (d, J=22.9 Hz, 3H), 1.55 (d, J=22.4 Hz, 3H). LCMS [M+H]⁺ or [M−H]⁻: 538.0.

(2-(3-((1R,2R)-1,2-difluoro-1-(4-methyl-4H-1,2,4-triazol-3-yl)propan-2-yl)phenyl)-6-((3-fluoro-3-methylazetidin-1-yl)methyl)-4-(trifluoromethyl)isoindolin-1-one (8.4 mg, 11% yield). (34)¹H NMR (400 MHz, DMSO-d₆) δ 8.51 (s, 1H), 8.06-7.89 (m, 4H), 7.49 (d, J=8.0 Hz, 1H), 7.27 (d, J=7.9 Hz, 1H), 6.41 (dd, J=43.5, 21.6 Hz, 1H), 5.29-5.11 (m, 2H), 3.87 (s, 2H), 3.50 (s, 3H), 1.82 (dd, J=23.5, 1.4 Hz, 3H), 1.62-1.50 (m, 3H). LCMS [M+H]⁺ or [M−H]⁻: 538.0.

### Example 35: Compound 47 and Compound 48

Compounds 47 and 48 can be synthesized as follows.

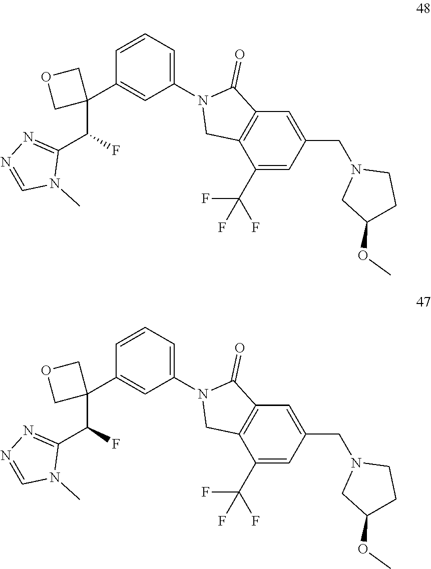 [SMILES: CO[C@@H]1CCN(Cc2cc3c(c(C(F)(F)F)c2)CN(c2cccc(C4([C@H](F)c5nncn5C)COC4)c2)C3=O)C1 ; CO[C@@H]1CCN(Cc2cc3c(c(C(F)(F)F)c2)CN(c2cccc(C4([C@@H](F)c5nncn5C)COC4)c2)C3=O)C1]

To 2-[3-[3-[fluoro-(4-methyl-1,2,4-triazol-3-yl)methyl]oxetan-3-yl]phenyl]-3-oxo-7-(trifluoromethyl)isoindoline-5-carbaldehyde (Intermediate I, Scheme 9, FIG. 5I, 110 mg, 0.23 mmol) in methanol (2.32 mL) were added (3R)-3-methoxypyrrolidine hydrochloride (63.8 mg, 0.46 mmol) and sodium acetate (77.0 mg, 0.93 mmol). The reaction mixture was stirred at rt for 15 min and then sodium triacetoxyborohydride (97.4 mg, 0.46 mmol) was added. The reaction was stirred at rt for 16 h. The reaction was diluted with saturated NaHCO₃ then concentrated almost to dryness. The residue was purified by chromatography on C18 silica gel (10-70% acetonitrile in ammonium formate, pH=4). Only the cleanest fraction were collected and lyophilized. After chiral separation (HPLC: Column.: ChiralPak IB, 250 mm×4.6 mm ID, 5 μm. mobile Phase: 8:12:80 8:12:80 MeOH:DCM:Hexane. Flow isocratic: 0.8 mL/min, (pressure 39 bars). Temp. Col.: ˜26° C. run time: 20 min.), desired products were obtained.

2-(3-(3-((R)-fluoro(4-methyl-4H-1,2,4-triazol-3-yl)methyl)oxetan-3-yl)phenyl)-6-(((R)-3-methoxypyrrolidin-1-yl)methyl)-4-(trifluoromethyl)isoindolin-1-one (20 mg, 15% yield), (47). ¹H NMR (400 MHz, DMSO-d₆) δ 8.35 (s, 1H), 7.98 (s, 1H), 7.96-7.90 (m, 2H), 7.56 (s, 1H), 7.38 (t, J=8.0 Hz, 1H), 6.97 (d, J=7.7 Hz, 1H), 6.28 (d, J=45.8 Hz, 1H), 5.37 (d, J=6.7 Hz, 1H), 5.22 (d, J=6.1 Hz, 1H), 5.18-5.03 (m, 3H), 4.83 (dd, J=6.0, 4.0 Hz, 1H), 3.96-3.85 (m, 1H), 3.83-3.70 (m, 2H), 3.18 (s, 3H), 3.16 (s, 3H), 2.69 (dd, J=10.0, 6.2 Hz, 1H), 2.61 (dd, J=14.9, 7.8 Hz, 1H), 2.46-2.39 (m, 1H), 2.07-1.95 (m, 1H), 1.74-1.62 (m, 1H). LCMS [M+H]⁺ or [M−H]⁻: 560.3.

2-(3-(3-((S)-fluoro(4-methyl-4H-1,2,4-triazol-3-yl)methyl)oxetan-3-yl)phenyl)-6-(((R)-3-methoxypyrrolidin-1-yl)methyl)-4-(trifluoromethyl)isoindolin-1-one (24 mg, 18% yield), (48). ¹H NMR (400 MHz, DMSO-d6) δ 8.35 (s, 1H), 7.98 (s, 1H), 7.96-7.90 (m, 2H), 7.55 (s, 1H), 7.38 (t, J=8.0 Hz, 1H), 6.97 (d, J=7.8 Hz, 1H), 6.28 (d, J=45.8 Hz, 1H), 5.37 (d, J=6.6 Hz, 1H), 5.21 (d, J=6.1 Hz, 1H), 5.18-5.03 (m, 3H), 4.83 (dd, J=6.0, 4.1 Hz, 1H), 3.99-3.84 (m, 1H), 3.85-3.72 (m, 2H), 3.18 (s, 3H), 3.16 (s, 3H), 2.69 (dd, J=10.0, 6.2 Hz, 1H), 2.61 (dd, J=15.0, 7.8 Hz, 2H), 2.46-2.37 (m, 1H), 2.12-1.93 (m, 1H), 1.76-1.62 (m, 1H). LCMS [M+H]⁺ or [M−H]⁻: 560.3.

### Example 36: Compound 49

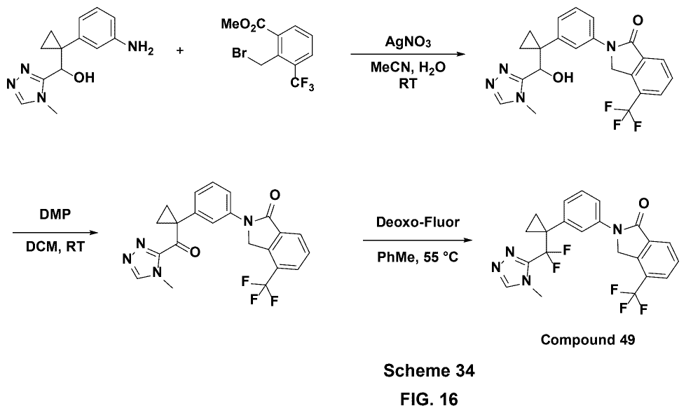

Compound 49 can be synthesized according to Scheme 34, FIG. 16.

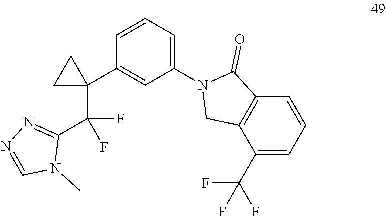 [SMILES: Cn1cnnc1C(F)(F)C1(c2cccc(N3Cc4c(cccc4C(F)(F)F)C3=O)c2)CC1]

A first intermediate, 2-(3-(1-(hydroxy(4-methyl-4H-1,2,4-triazol-3-yl)methyl)-cyclopropyl)phenyl)-4-(trifluoromethyl)isoindolin-1-one, was synthesized as follows. A mixture of (1-(3-aminophenyl)cyclopropyl)(4-methyl-4H-1,2,4-triazol-3-yl)methanol (an intermediate of scheme 4, FIG. 6D; 460 mg, 1.88 mmol) and methyl 2-(bromomethyl)-3-(trifluoromethyl)benzoate (559 mg, 1.88 mmol) in acetonitrile (15 mL) and water (7.5 mL) was cooled to 0° C., then a solution of silver nitrate (416 mg, 2.45 mmol) in water (7.5 mL) was added drop wise. The reaction was stirred for 18 h at RT. A saturated solution of sodium carbonate was added until the pH reached ˜9. The mixture was diluted with methanol (25 mL), filtered over celite (washed with 300 mL MeOH) and the filtrate was concentrated to remove the methanol. The aqueous residue was diluted with 4:1 CHCl₃/MeOH (50 mL) and the layers were separated. The organic phases were extracted with 4:1 CHCl₃/MeOH (4×50 mL). The organic phases were combined, dried with magnesium sulfate, filtered and evaporated under reduced pressure. The residue was purified by chromatography on silica gel (2-10% methanol in CH₂Cl₂) to afford 2-(3-(1-(hydroxy(4-methyl-4H-1,2,4-triazol-3-yl)methyl)cyclopropyl)phenyl)-4-(trifluoromethyl)iso-indolin-1-one (402 mg, 50%). LCMS (ESI) m/z: 429.1 [M+H]⁺.

A second intermediate, 2-(3-(1-(4-methyl-4H-1,2,4-triazole-3-carbonyl)cyclo-propyl)phenyl)-4-(trifluoromethyl)isoindolin-1-one, was made as follows. DMP (796 mg, 1.88 mmol) was added in one portion to a solution of 2-(3-(1-(hydroxy(4-methyl-4H-1,2,4-triazol-3-yl)methyl)cyclopropyl)phenyl)-4-(trifluoromethyl)isoindolin-1-one (402 mg, 0.938 mmol) in DCM (19 mL) at RT. The resulting mixture was stirred for 20 h at the same temperature. The reaction was quenched with sat. aqueous sodium bicarbonate (5 mL) and sat. aqueous sodium thiosulfate (5 mL) and stirred for 5 min. The reaction was diluted 4:1 CHCl₃/IPA (20 mL). The layers were separated, the aqueous phase was extracted with 4:1 CHCl₃/IPA (3×20 mL), the combined organic phases were dried over magnesium sulfate, filtered and concentrated under reduced pressure to afford 2-(3-(1-(4-methyl-4H-1,2,4-triazole-3-carbonyl)cyclopropyl)phenyl)-4-(trifluoromethyl)isoindolin-1-one (412 mg, 100% yield). LCMS (ESI) m/z: 427.2 [M+H]⁺.

In a final step, Compound 49 was obtained as follows. (bis-(2-methoxyethyl)-amino)sulfur trifluoride 50% in PhMe (1.56 mL, 3.52 mmol) was added to 2-(3-(1-(4-methyl-4H-1,2,4-triazole-3-carbonyl)cyclopropyl)phenyl)-4-(trifluoromethyl)isoindolin-1-one (100 mg, 0.230 mmol) at 0° C. The reaction was allowed to warm to RT and stirred for 24 h at 55° C. The reaction was carefully quenched at 0° C. with sat. aqueous potassium carbonate until pH 8 was reached. The mixture was diluted with 4:1 CHCl₃/IPA (25 mL). The layers were separated, the aqueous phase was extracted with 4:1 CHCl₃/IPA (2×25 mL), the combined organic phases were dried over magnesium sulfate, filtered and concentrated under reduced pressure. The residue was purified by Preparative HPLC to afford a product not sufficiently pure.

The product was further purified by flash column chromatography (0-4% gradient of MeOH in DCM) to afford 2-(3-(1-(difluoro(4-methyl-4H-1,2,4-triazol-3-yl)methyl)-cyclopropyl)phenyl)-4-(trifluoromethyl)isoindolin-1-one (21 mg, 20%), (49). ¹H NMR (400 MHz, DMSO-d₆) δ 8.50 (s, 1H), 8.08 (d, J=7.6 Hz, 1H), 8.05 (d, J=7.7 Hz, 1H), 7.90 (s, 1H), 7.85-7.78 (m, 2H), 7.37 (t, J=7.9 Hz, 1H), 7.17 (d, J=7.6 Hz, 1H), 5.15 (s, 2H), 3.38 (s, 3H), 1.43 (dd, J=5.1, 6.8 Hz, 2H), 1.18-1.13 (m, 2H). LCMS [M+H]⁺ or [M−H]⁻=449.2.

### Example 37: Compound 50

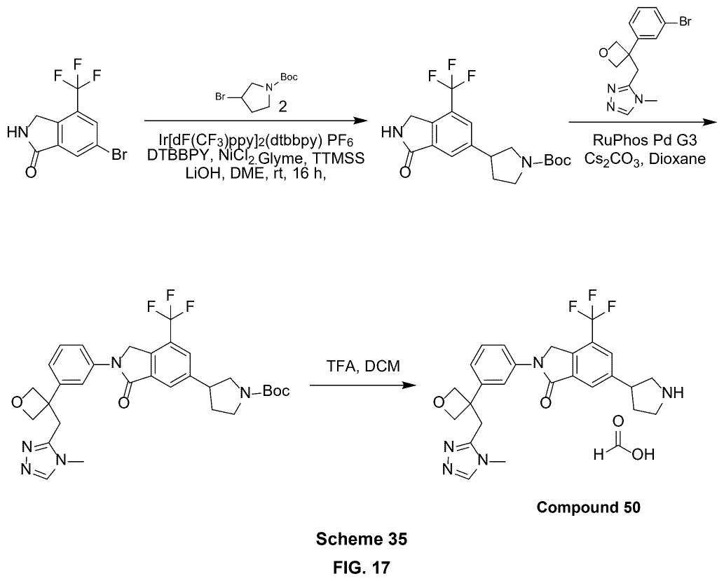

Compound 50, (±)-2-(3-(3-((4-methyl-4H-1,2,4-triazol-3-yl)methyl)oxetan-3-yl)phenyl)-6-(pyrrolidin-3-yl)-4-(trifluoromethyl)isoindolin-1-one formate, can be synthesized according to Scheme 35, FIG. 17.

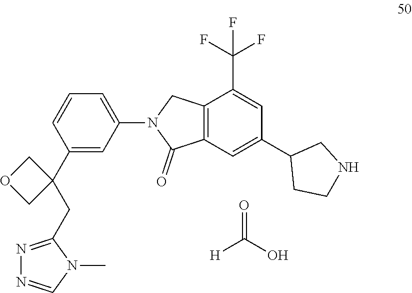 [SMILES: Cn1cnnc1CC1(c2cccc(N3Cc4c(cc(C5CCNC5)cc4C(F)(F)F)C3=O)c2)COC1 ; O=CO]

A first intermediate, tert-butyl 3-(3-oxo-7-(trifluoromethyl)isoindolin-5-yl)pyrrolidine-1-carboxylate, was made as follows. To a mixture of tert-butyl-bromo-1-pyrrolidinecarboxylate (268.0 mg, 1.07 mmol), 6-bromo-4-(trifluoromethyl)isoindolin-1-one (200.0 mg, 0.71 mmol) in 1,2-dimethoxyethane (20 mL) was added lithium hydroxide (42.7 mg, 1.79 mmol), Ir[dF(CF₃)ppy]2(dtbbpy)PF₆ (8.0 mg, 0.01 mmol), tris(trimethylsilyl)silane (213.1 mg, 0.86 mmol). Then a solution of nickel(II) chloride ethylene glycol dimethyl ether complex (15.7 mg, 0.07 mmol) and 4,4′-di-tert-butyl-2,2′-dipyridyl (28.7 mg, 0.11 mmol) in 1,2-dimethoxyethane (2 mL) was added to the mixture solution in a glove box at 20° C. The reaction mixture was stirred under a Lumidox Screen Kit for 16 h at 20° C. The reaction mixture was diluted with water (10 mL) and extracted with ethyl acetate (3×20 mL). The combined organic layers were dried over sodium sulfate and concentrated. The residue was purified by RP-HPLC (water (0.2% formic acid)-ACN 43% to 73%) to give tert-butyl 3-(3-oxo-7-(trifluoromethyl)isoindolin-5-yl)pyrrolidine-1-carboxylate (82 mg, 31% yield) as a white solid. LCMS [M+H-56]⁺=315.0.

A second intermediate, tert-butyl 3-(2-(3-(3-((4-methyl-4H-1,2,4-triazol-3-yl)-methyl)oxetan-3-yl)phenyl)-3-oxo-7-(trifluoromethyl)isoindolin-5-yl) pyrrolidine-1-carboxylate, was made as follows. Under nitrogen, a mixture of 3-[[3-(3-bromophenyl)oxetan-3-yl]methyl]-4-methyl-1,2,4-triazole (synthesized as shown in WO2019148005; 61.0 mg, 0.20 mmol), tert-butyl 3-[3-oxo-7-(trifluoromethyl)isoindolin-5-yl]pyrrolidine-1-carboxylate (80.6 mg, 0.22 mmol), cesium carbonate (193.48 mg, 0.59 mmol), (2-dicyclohexylphosphino-2′,6′-diisopropoxy-1,1′-biphenyl)[2-(2′-amino-1,1′-biphenyl)]palladium(ii) methanesulfonate (49.7 mg, 0.06 mmol) in 1,4-dioxane (3 mL) was stirred for 16 h at 110° C. After cooling, the reaction mixture was filtered. The filtrate was concentrated under reduced pressure. The residue was purified by preparative TLC (mobile phase: 12% methanol in ethyl acetate) to afford tert-butyl 3-(2-(3-(3-((4-methyl-4H-1,2,4-triazol-3-yl)methyl)oxetan-3-yl)phenyl)-3-oxo-7-(trifluoromethyl)isoindolin-5-yl) pyrrolidine-1-carboxylate (72 mg, 61% yield) as a light-yellow solid. LCMS [M+H]+=598.0.

Compound 50 is then obtained as follows. To a mixture of tert-butyl 3-[2-[3-[3-[(4-methyl-1,2,4-triazol-3-yl)methyl]oxetan-3-yl]phenyl]-3-oxo-7-(trifluoromethyl)isoindolin-5-yl]pyrrolidine-1-carboxylate (72 mg, 0.12 mmol) in dichloromethane (5 mL) was added trifluoroacetic acid (0.5 mL, 0.50 mmol), the mixture was stirred at 25° C. for 1 h. The mixture was quench by NH₄OH (1 ML) and concentrated to dryness under reduced pressure. The residue was purified by RP-HPLC (water (0.2% formic acid)-ACN 7% to 37%) to afford 2-(3-(3-((4-methyl-4H-1,2,4-triazol-3-yl)methyl)oxetan-3-yl)phenyl)-6-(pyrrolidin-3-yl)-4-(trifluoromethyl)isoindolin-1-one formate (18 mg, 27.5% yield) as a white solid, (50), a racemate. ¹H NMR (400 MHz, methanol-d₄): δ 8.39 (br s, 1H), 8.16 (s, 1H), 8.05 (s, 1H), 7.95 (s, 1H), 7.74 (dd, J=1.2, 7.6 Hz, 1H), 7.40 (t, J=7.6 Hz, 1H), 7.35 (t, J=1.6 Hz, 1H), 6.78 (d, J=7.2 Hz, 1H), 5.08-5.03 (m, 6H), 4.60 (br s, 1H), 3.83-3.74 (m, 2H), 3.64-3.58 (m, 3H), 3.45-3.37 (m, 1H), 2.88 (s, 3H), 2.59-2.54 (m, 1H), 2.23-2.12 (m, 1H). LCMS [M+H]⁺ or [M−H]⁻=498.1.

### Example 38: Compounds 51 and 52

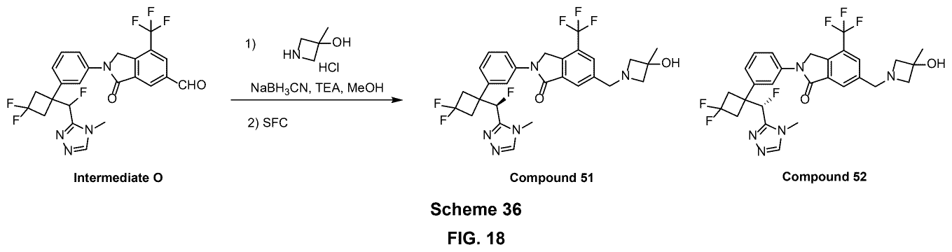

Compounds 51 and 52 can be synthesized according to Scheme 36, FIG. 18.

To a solution of 2-(3-(3,3-difluoro-1-(fluoro(4-methyl-4H-1,2,4-triazol-3-yl)methyl)cyclobutyl)phenyl)-3-oxo-7-(trifluoromethyl)isoindoline-5-carbaldehyde (Intermediate O, Scheme 15, FIG. 60; 100.0 mg, 0.2 mmol) and 3-methylazetidin-3-ol hydrochloride (48.6 mg, 0.4 mmol) in methanol (3 mL) was added triethylamine (32.9 μL, 0.2 mmol). The mixture was stirred at 100° C. for 1 minute under microwave irridation. The mixture was then added sodium cyanoborohydride (24.7 mg, 0.4 mmol) and heated at 80° C. under microwave irridation for another 45 minutes. The reaction mixture was quenched by 1 M HCl aqueous (2 mL) and adjusted to pH=6˜7 with saturated aqueous NaHCO₃. The reaction mixture was concentrated under vacuum. The residue was purified by RP-HPLC (35% to 65% ACN/(0.05% NH₃H₂O+10 mM NH₄HCO₃ in water)) to afford 2-(3-(3,3-difluoro-1-(fluoro(4-methyl-4H-1,2,4-triazol-3-yl)methyl)cyclobutyl)phenyl)-6-((3-hydroxy-3-methylazetidin-1-yl)methyl)-4-(trifluoromethyl)isoindolin-1-one (24 mg, 21.1% yield) as a white solid.

The above racemate (24 mg, 0.04 mmol) was further purified by chiral SFC (Column=Daicel Chiralcel OD; Column dimensions=250 mm×30 mm×10 μm; Detection wavelength=220 nM; Flow rate=70 mL/min; Run time=4.0 min; Column temperature=25° C.) with 0.1% ammonium hydroxide—30% ethanol—carbon dioxide) to afford Compounds 51 and 52, characterized as follows.

(R)-2-(3-(3,3-difluoro-1-(fluoro(4-methyl-4H-1,2,4-triazol-3-yl)methyl)cyclobutyl)-phenyl)-6-((3-hydroxy-3-methylazetidin-1-yl)methyl)-4-(trifluoromethyl)isoindolin-1-one (Peak 1, retention time=1.391 min) (8.92 mg, 44.2% yield) as a white solid, (51). ¹H NMR (400 MHz, methanol-d₄) δ 8.22 (s, 1H), 8.00 (s, 1H), 7.90 (s, 1H), 7.75 (dd, J=1.6, 8.4 Hz, 1H), 7.57 (br s, 1H), 7.40 (t, J=8.0 Hz, 1H), 6.96 (d, J=8.0 Hz, 1H), 6.04 (d, J=44.8 Hz, 1H), 5.07 (s, 2H), 3.86 (s, 2H), 3.62-3.44 (m, 2H), 3.34 (d, J=8.8 Hz, 2H), 3.20-3.05 (m, 4H), 2.90 (s, 3H), 1.45 (s, 3H). LCMS [M+H]⁺ or [M−H]⁻=580.1.

(S)-2-(3-(3,3-difluoro-1-(fluoro(4-methyl-4H-1,2,4-triazol-3-yl)methyl)cyclobutyl)-phenyl)-6-((3-hydroxy-3-methylazetidin-1-yl)methyl)-4-(trifluoromethyl)isoindolin-1-one (Peak 2, retention time=1.579 min) (3 mg, 15% yield) as a white solid, (52). ¹H NMR (400 MHz, methanol-d₄) δ 8.23 (s, 1H), 8.00 (s, 1H), 7.90 (s, 1H), 7.75 (dd, J=1.2, 8.4 Hz, 1H), 7.57 (br s, 1H), 7.40 (t, J=8.0 Hz, 1H), 6.96 (d, J=7.6 Hz, 1H), 6.04 (d, J=44 Hz, 1H), 5.07 (s, 2H), 3.85 (s, 2H), 3.63-3.47 (m, 2H), 3.34 (d, J=8.0 Hz, 2H), 3.17-3.05 (m, 4H), 2.91 (s, 3H), 1.46 (s, 3H). LCMS [M+H]⁺ or [M−H]⁻=580.1.

### Example 39: Compounds 53 and 54

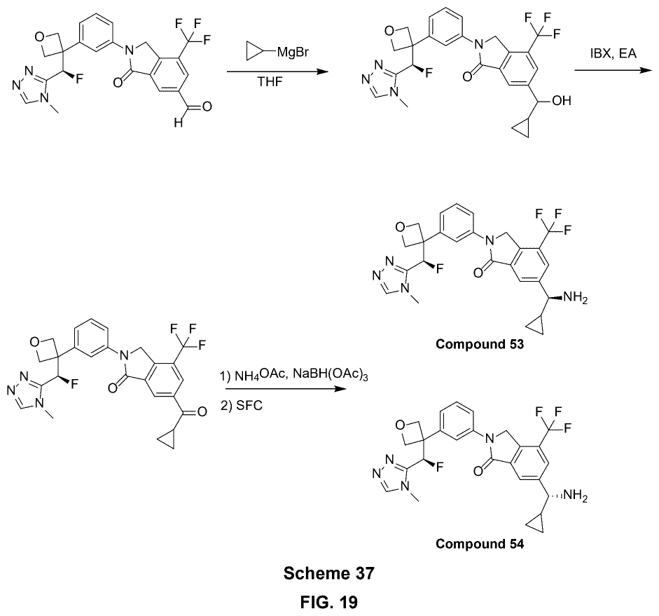

Compounds 53 and 54 can be synthesized according to Scheme 37, FIG. 19.

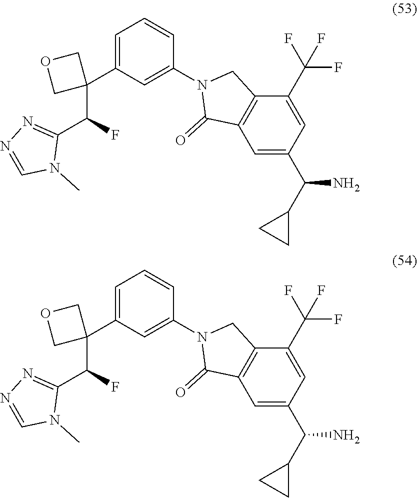

Intermediate 2, 6-(cyclopropyl(hydroxy)methyl)-2-(3-(3-((R)-fluoro(4-methyl-4H-1,2,4-triazol-3-yl)methyl)oxetan-3-yl)phenyl)-4-(trifluoromethyl)isoindolin-1-one, was made as follows. To a mixture of (R)-2-(3-(3-(fluoro(4-methyl-4H-1,2,4-triazol-3-yl)methyl)oxetan-3-yl)phenyl)-3-oxo-7-(trifluoromethyl)isoindoline-5-carbaldehyde (Intermediate I, Scheme 9, FIG. 61; 200.0 mg, 0.42 mmol) in tetrahydrofuran (3.5 mL) was added cyclopropyl magnesiumbromide (0.5M in tetrahydrofuran, 1.69 mL, 0.84 mmol) at −78° C. After addition, the resulting mixture was stirred at −78° C. for 2 h and quenched by addition of saturated aqueous NH₄Cl (10 mL). The reaction mixture was diluted with dichloromethane (20 mL), washed with brine (30 mL), dried over with sodium sulfate and concentrated to dryness. The residue was purified by silica gel chromatography (mobile phase: methanol/dichloro-methane, gradient 0% to 25%) to afford 6-(cyclopropyl(hydroxy)methyl)-2-(3-(3-((R)-fluoro(4-methyl-4H-1,2,4-triazol-3-yl)methyl)oxetan-3-yl)phenyl)-4-(trifluoromethyl)isoindolin-1-one (200.0 mg, 91.9% yield) as a yellow oil. LCMS: [M+H]⁺=517.0.

A second intermediate, (R)-6-(cyclopropanecarbonyl)-2-(3-(3-(fluoro(4-methyl-4H-1,2,4-triazol-3-yl)methyl)oxetan-3-yl)phenyl)-4-(trifluoromethyl)isoindolin-1-one, was obtained as follows. To a stirred solution of 2-iodoxybenzoic acid (542.2 mg, 1.94 mmol) in ethyl acetate (3 mL) was added 6-(cyclopropyl(hydroxy)methyl)-2-(3-(3-((R)-fluoro(4-methyl-4H-1,2,4-triazol-3-yl)methyl)oxetan-3-yl)phenyl)-4-(trifluoromethyl)isoindolin-1-one (200.0 mg, 0.39 mmol). The reaction was stirred at 75° C. for 5 h and filtered. The filtrate was concentrated to give crude (R)-6-(cyclopropanecarbonyl)-2-(3-(3-(fluoro(4-methyl-4H-1,2,4-triazol-3-yl)methyl)oxetan-3-yl)phenyl)-4-(trifluoromethyl)isoindolin-1-one (120 mg, 60.2% yield) as white solid, which was used directly for next step. LCMS: [M+H]⁺=515.0.

To a solution of (R)-6-(cyclopropanecarbonyl)-2-(3-(3-(fluoro(4-methyl-4H-1,2,4-triazol-3-yl)methyl)oxetan-3-yl)phenyl)-4-(trifluoromethyl)isoindolin-1-one (70.0 mg, 0.14 mmol) and ammonium acetate (104.9 mg, 1.36 mmol) in methanol (2 mL) was added sodium cyanoborohydride (21.4 mg, 0.34 mmol). The mixture was heated to 70° C. and stirred for 12 h. The reaction was quenched with 1 M HCl solution and adjusted to pH=7 by addition of saturated NaHCO₃ solution. The resulting solution was extracted with dichloromethane (3×20 mL). The combined organic phases were dried over anhydrous sodium sulfate and concentrated under reduced pressure. The resulting residue was purified by RP-HPLC (35% to 60% ACN/(0.05% NH₃H₂O+10 mM NH₄HCO₃ in water)) to afford 6-(amino(cyclopropyl)methyl)-2-(3-(3-((R)-fluoro(4-methyl-4H-1,2,4-triazol-3-yl)methyl)oxetan-3-yl)phenyl)-4-(trifluoro-methyl)isoindolin-1-one (21 mg, 29% yield) as a yellow solid. LCMS [M+H]⁺=516.3.

The above racemate was further purified by chiral SFC (Column=(SS)Whelk-01; Column dimensions=250 mm×30 mm×5 μm; Detection wavelength=220 nm, Flow rate=80 mL/min; Run time=7 min; Column temperature=40° C.) with 0.1% ammonium hydroxide-35% ethanol-carbon dioxide) to afford Compounds 53 and 54.

6-((S)-amino(cyclopropyl)methyl)-2-(3-(3-((R)-fluoro(4-methyl-4H-1,2,4-triazol-3-yl)methyl)oxetan-3-yl)phenyl)-4-(trifluoromethyl)isoindolin-1-one (Peak 1, retention time=3.445 min) (3.46 mg, 15.6% yield) as a white solid, (53). ¹H NMR (400 MHz, CDCl₃) δ 8.15 (br s, 1H), 8.01 (s, 1H), 7.91 (s, 1H), 7.64 (d, J=8.0 Hz, 1H), 7.47 (br s, 1H), 7.38 (t, J=8.0 Hz, 1H), 6.78 (d, J=7.6 Hz, 1H), 6.47 (d, J=46.0 Hz, 1H), 5.34-5.28 (m, 2H), 5.25-5.23 (m, 1H), 5.02-4.91 (m, 3H), 3.42-3.39 (m, 1H), 3.03 (d, J=1.6 Hz, 3H), 1.13-1.09 (m, 1H), 0.72-0.68 (m, 1H), 0.58-0.55 (m, 1H), 0.46-0.42 (m, 1H), 0.42-0.34 (m, 1H). LCMS [M+H]⁺ or [M−H]⁻=516.1.

6-((R)-amino(cyclopropyl)methyl)-2-(3-(3-((R)-fluoro(4-methyl-4H-1,2,4-triazol-3-yl)methyl)oxetan-3-yl)phenyl)-4-(trifluoromethyl)isoindolin-1-one (Peak 2, retention time=4.513 min) (4.46 mg, 19.1% yield) as a white solid, (54). ¹H NMR (400 MHz, CDCl₃) δ 8.19 (br s, 1H), 8.01 (s, 1H), 7.93 (s, 1H), 7.63 (d, J=7.6 Hz, 1H), 7.47 (br s, 1H), 7.38 (t, J=8.0 Hz, 1H), 6.79 (d, J=7.6 Hz, 1H), 6.47 (d, J=46 Hz, 1H), 5.34-5.28 (m, 2H), 5.25-5.23 (m, 1H), 5.02-4.90 (m, 3H), 3.47-3.42 (m, 1H), 3.03 (d, J=1.6 Hz, 3H), 1.19-1.17 (m, 1H), 0.75-0.69 (m, 1H), 0.61-0.55 (m, 1H), 0.50-0.47 (m, 1H), 0.41-0.35 (m, 1H)). LCMS [M+H]⁺ or [M−H]⁻=516.0.

### Example 40: Compound 55

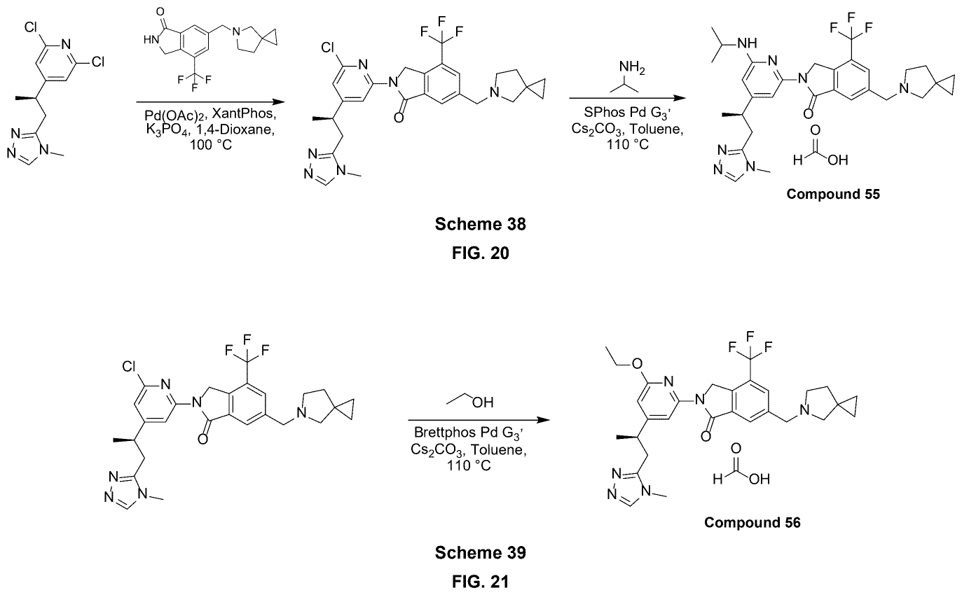

Compound 55 can be synthesized according to Scheme 38, FIG. 20.

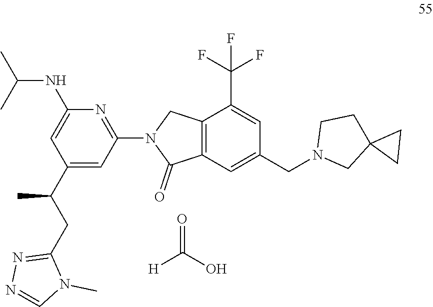

A first intermediate, (R)-6-((5-azaspiro[2.4]heptan-5-yl)methyl)-2-(6-chloro-4-(1-(4-methyl-4H-1,2,4-triazol-3-yl)propan-2-yl)pyridin-2-yl)-4-(trifluoromethyl)isoindolin-1-one, was made as follows. To a mixture of (R)-2,6-dichloro-4-(1-(4-methyl-4H-1,2,4-triazol-3-yl)propan-2-yl)pyridine (317.2 mg, 1.17 mmol) and 6-((5-azaspiro[2.4]heptan-5-yl)methyl)-4-(trifluoromethyl)isoindolin-1-one (synthesized as shown in WO2019148005, at Example AB, p. 344; 330.0 mg, 1.06 mmol) in 1,4-dioxane (15 mL) was added palladium(II) acetate (47.7 mg, 0.21 mmol), 4,5-bis(diphenylphosphino)-9,9-dimethylxanthene (123.1 mg, 0.21 mmol) and potassium phosphate (451.5 mg, 2.13 mmol). The mixture was stirred under nitrogen for 16 h at 100° C. After cooling to room temperature, the reaction was concentrated under reduced pressure. The residue was purified by silica gel chromatography (mobile phase: methanol/dichloromethane, gradient 0% to 10%) to afford (R)-6-((5-azaspiro[2.4]heptan-5-yl)methyl)-2-(6-chloro-4-(1-(4-methyl-4H-1,2,4-triazol-3-yl)propan-2-yl)pyridin-2-yl)-4-(trifluoromethyl)isoindolin-1-one (350 mg, 60.4% yield) as a light yellow oil. LCMS: [M+H]⁺=545.1.

A mixture of (R)-6-((5-azaspiro[2.4]heptan-5-yl)methyl)-2-(6-chloro-4-(1-(4-methyl-4H-1,2,4-triazol-3-yl)propan-2-yl)pyridin-2-yl)-4-(trifluoromethyl)isoindolin-1-one (130.0 mg, 0.24 mmol), isopropylamine (0.04 mL, 0.48 mmol), (2-dicyclohexylphosphino-2′,6′-dimethoxybiphenyl) [2-(2′-amino-1,1′-biphenyl)]palladium(II) methanesulfonate (27.9 mg, 0.04 mmol), cesium carbonate (233.16 mg, 0.72 mmol) in toluene (3 mL) was stirred under nitrogen for 16 h at 110° C. and concentrated to dryness under reduced pressure to yield Compound 55.

The residue was purified by RP-HPLC (29% to 59% ACN/0.2% formic acid in water) to afford (R)-6-((5-azaspiro[2.4]heptan-5-yl)methyl)-2-(6-(isopropylamino)-4-(1-(4-methyl-4H-1,2,4-triazol-3-yl)propan-2-yl)pyridin-2-yl)-4-(trifluoromethyl)isoindolin-1-one formate (13.8 mg, 9.2% yield) as a yellow solid, (55), characterized as follows. LCMS: [M+H]⁺=568.1. ¹H NMR (400 MHz, CDCl₃) δ 8.35 (s, 1H), 8.06 (s, 1H), 7.99 (s, 1H), 7.98 (s, 1H), 7.79 (s, 1H), 5.96 (s, 1H), 5.25-5.07 (m, 2H), 3.98 (s, 2H), 3.89-3.83 (m, 1H), 3.48 (s, 3H), 3.37-3.32 (m, 1H), 3.07-2.97 (m, 4H), 2.72 (s, 2H), 1.94-1.90 (m, 2H), 1.41 (d, J=6.4 Hz, 3H), 1.25-1.22 (m, 6H), 0.62 (s, 4H). LCMS [M+H]⁺ or [M−H]⁻=568.1.

### Example 41: Compound 56

Compound 56 can be synthesized according to Scheme 39, FIG. 21.

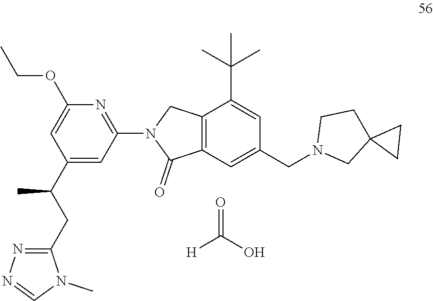

To a mixture of (R)-6-((5-azaspiro[2.4]heptan-5-yl)methyl)-2-(6-chloro-4-(1-(4-methyl-4H-1,2,4-triazol-3-yl)propan-2-yl)pyridin-2-yl)-4-(trifluoromethyl)isoindolin-1-one, the first intermediate in Example 40, (100.0 mg, 0.18 mmol), ethanol (0.12 mL, 1.83 mmol), methanesulfonato(2-dicyclohexylphosphino-3,6-dimethoxy-2′,4′,6′-tri-i-propyl-1,1′-biphenyl)(2′-amino-1,1′-biphenyl-2-yl)palladium(II) (27.9 mg, 0.04 mmol), cesium carbonate (179.3 mg, 0.55 mmol) in toluene (1 mL) was stirred for 1 h at 110° C. under microwave irradiation, after cooled to room temperature, the reaction mixture was concentrated to dryness under reduced pressure.

The residue was purified by RP-HPLC (22% to 52% ACN/0.2% formic acid in water) to afford (R)-6-((5-azaspiro[2.4]heptan-5-yl)methyl)-2-(6-ethoxy-4-(1-(4-methyl-4H-1,2,4-triazol-3-yl)propan-2-yl)pyridin-2-yl)-4-(trifluoromethyl)isoindolin-1-one formate (17.78 mg, 16.6% yield) as a yellow solid, (56). LCMS: [M+H]⁺=555.2. ¹H NMR (400 MHz, CDCl₃) δ 8.26 (s, 1H), 8.09 (s, 1H), 8.06 (s, 1H), 8.02 (s, 2H), 6.41 (s, 1H), 5.27-5.15 (m, 2H), 4.35 (q, J=7.2 Hz, 2H), 4.08 (s, 2H), 3.54 (s, 3H), 3.48-3.40 (m, 1H), 3.12-3.05 (m, 4H), 2.81 (s, 2H), 1.97-1.93 (m, 2H), 1.46-1.40 (m, 6H), 0.64 (s, 4H). LCMS [M+H]⁺ or [M−H]⁻=555.2.

### Example 42: Compounds 57 and 58

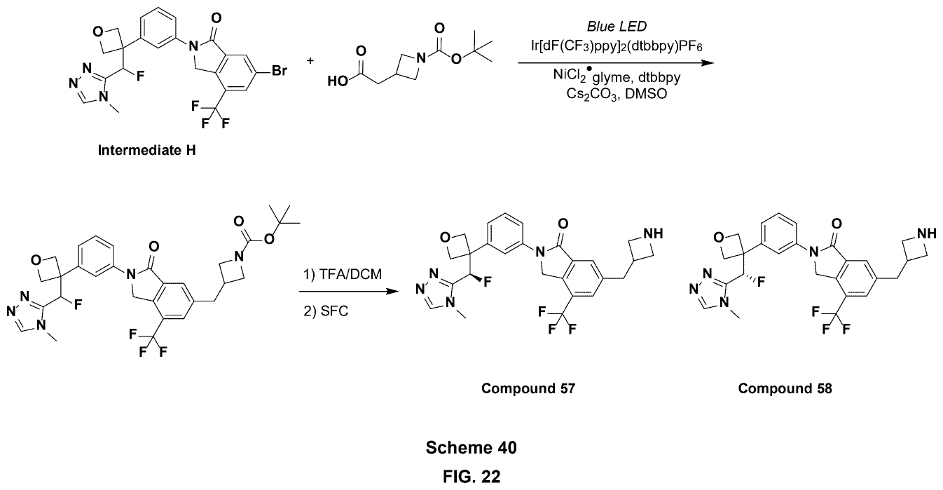

Compounds 57 and 58 can be synthesized according to Scheme 40, FIG. 22.

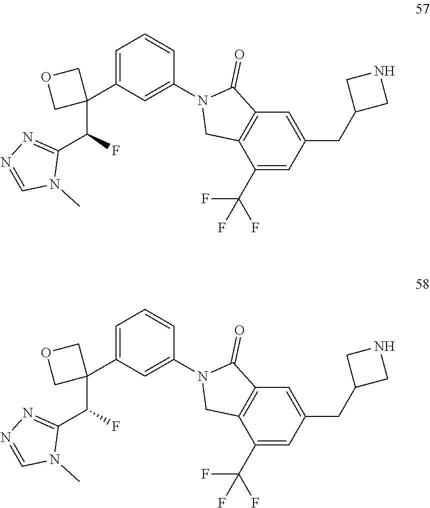 [SMILES: Cn1cnnc1[C@H](F)C1(c2cccc(N3Cc4c(cc(CC5CNC5)cc4C(F)(F)F)C3=O)c2)COC1 ; Cn1cnnc1[C@@H](F)C1(c2cccc(N3Cc4c(cc(CC5CNC5)cc4C(F)(F)F)C3=O)c2)COC1]

A first intermediate, tert-Butyl 3-((2-(3-(3-(fluoro(4-methyl-4H-1,2,4-triazol-3-yl)methyl)oxetan-3-yl)phenyl)-3-oxo-7-(trifluoromethyl)isoindolin-5-yl)methyl)azetidine-1-carboxylate, was made as follows. A vial was charged with 6-bromo-2-(3-(3-(fluoro(4-methyl-4H-1,2,4-triazol-3-yl)methyl)oxetan-3-yl)phenyl)-4-(trifluoromethyl)isoindolin-1-one (Intermediate H, 100 mg, 0.190 mmol), 2-(1-(tert-butoxycarbonyl)azetidin-3-yl)acetic acid (123 mg, 0.571 mmol), Ir[dF(CF₃)ppy]₂(dtbbpy)PF₆ (10.7 mg, 0.0095 mmol), nickel(II) chloride ethylene glycol dimethyl ether complex (2.1 mg, 0.0095 mmol), 4,4′-di-tert-butyl-2,2′-dipyridyl (2.6 mg, 0.0095 mmol) and cesium carbonate (125 mg, 0.381 mmol). The vial was purged with nitrogen before degassed DMSO (10 mL) was added. Nitrogen was bubbled into the reaction mixture for 10 min and the vial was sealed. The reaction was stirred under a blue LED light irradiation (100% intensity) for 2 h. The reaction mixture was diluted with EtOAc (50 mL) and water (50 mL) and the layers were separated. The aqueous phase extracted with EtOAc (50 mL), the combined organic phases were washed with water, washed with brine, dried with magnesium sulfate, filtered and evaporated under reduced pressure. The residue was purified by chromatography on silica gel (0-10% methanol in CH₂Cl₂) to afford tert-butyl 3-((2-(3-(3-(fluoro(4-methyl-4H-1,2,4-triazol-3-yl)methyl)oxetan-3-yl)phenyl)-3-oxo-7-(trifluoromethyl)isoindolin-5-yl)methyl)azetidine-1-carboxylate (22 mg, 19%). LCMS (ESI) m/z: 616.3 [M+H]⁺.

Trifluoroacetic acid (0.40 mL) was added to a solution of tert-butyl 3-((2-(3-(3-(fluoro(4-methyl-4H-1,2,4-triazol-3-yl)methyl)oxetan-3-yl)phenyl)-3-oxo-7-(trifluoromethyl)-isoindolin-5-yl)methyl)azetidine-1-carboxylate (31 mg, 0.0097 mmol) in DCM (4.0 mL) at rt. The resulting mixture was stirred for 1 h before triethylamine (1 mL) was added. The resulting mixture was concentrated under reduced. The residue was purified by chromatography on C18 silica gel (10-60% acetonitrile in ammonium formate, pH=3.8). After chiral separation (SFC: Column: Lux Cel-4, 10×250 mm 5 um, Mode: Isocratic, Mobile phase: 60% MeOH+0.1% NH4OH, 40% supercritical CO₂, Flow rate: 10 mL/min, Backpressure: 150 bar, Column Temperature: 40° C., Run time (min): 14), desired products were obtained and characterized as follows.

(R)-6-(azetidin-3-ylmethyl)-2-(3-(3-(fluoro(4-methyl-4H-1,2,4-triazol-3-yl)methyl)oxetan-3-yl)phenyl)-4-(trifluoromethyl)isoindolin-1-one (5 mg, 26% yield), (57). ¹H NMR (400 MHz, DMSO-d6) 8.32 (s, 1H), 7.95-7.82 (m, 3H), 7.52 (s, 1H), 7.35 (t, J=8.0 Hz, 1H), 6.94 (d, J=7.8 Hz, 1H), 6.25 (d, J=45.8 Hz, 1H), 5.34 (d, J=6.8 Hz, 1H), 5.19 (d, J=6.2 Hz, 1H), 5.07 (dd, J=15.7, 8.5 Hz, 3H), 4.84-4.76 (m, 1H), 3.43-3.38 (m, 2H), 3.15 (s, 3H), 3.04 (d, J=7.8 Hz, 2H). LCMS [M+H]⁺ or [M−H]⁻=516.4.

(S)-6-(azetidin-3-ylmethyl)-2-(3-(3-(fluoro(4-methyl-4H-1,2,4-triazol-3-yl)methyl)oxetan-3-yl)phenyl)-4-(trifluoromethyl)isoindolin-1-one (5 mg, 26% yield), (58). ¹H NMR (400 MHz, DMSO-d6) 8.32 (s, 1H), 7.95-7.82 (m, 3H), 7.52 (s, 1H), 7.35 (t, J=8.0 Hz, 1H), 6.94 (d, J=7.8 Hz, 1H), 6.25 (d, J=45.8 Hz, 1H), 5.34 (d, J=6.8 Hz, 1H), 5.19 (d, J=6.2 Hz, 1H), 5.07 (dd, J=15.7, 8.5 Hz, 3H), 4.84-4.76 (m, 1H), 3.43-3.38 (m, 2H), 3.15 (s, 3H), 3.04 (d, J=7.8 Hz, 2H). LCMS [M+H]⁺ or [M−H]⁻=516.4.

### Example 43: Compound 59

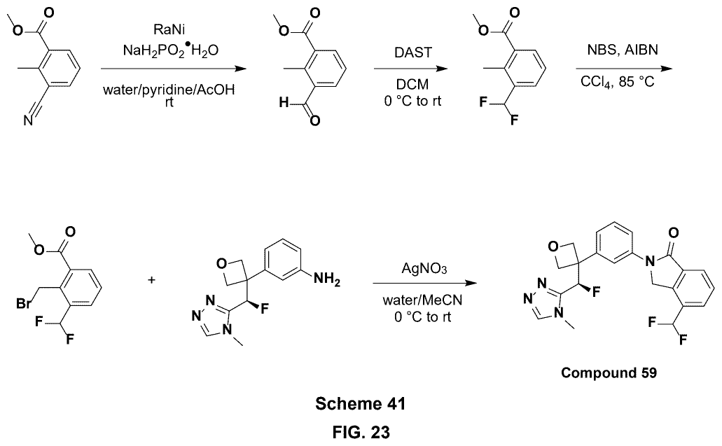

Compound 59 can be synthesized according to Scheme 41, FIG. 23.

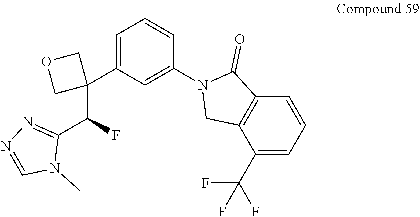 [SMILES: Cn1cnnc1[C@H](F)C1(c2cccc(N3Cc4c(cccc4C(F)(F)F)C3=O)c2)COC1]

A first intermediate, methyl 3-formyl-2-methylbenzoate, was made as follows. To a mixture of methyl 3-cyano-2-methylbenzoate (500 mg, 2.85 mmol), water (3 mL), pyridine (6 mL) and acetic acid (3 mL) was added sodium hypophosphite monohydrate (2.37 g, 22.8 mmol) at rt, followed by Raney®-Nickel (147 mg, 1.71 mmol). The resulting mixture was stirred at rt for 16 h. The reaction mixture was diluted with EtOAc (50 mL) and water (50 mL) and the layers were separated. The aqueous phase extracted with EtOAc (50 mL), the combined organic phases were washed with 1 N HCl, washed with brine, dried with magnesium sulfate, filtered and evaporated under reduced pressure to afford methyl 3-formyl-2-methylbenzoate (500 mg, 98%).

A second intermediate, methyl 3-(difluoromethyl)-2-methylbenzoate, was made as follows. To a solution of methyl 3-formyl-2-methyl-benzoate (250 mg, 1.40 mmol) in DCM (5.6 mL) at 0° C. was added DAST (0.93 mL, 7.02 mmol). The reaction was stirred at for 2 days at rt. The reaction mixture was diluted with DCM (10 mL) and water (10 mL) and the layers were separated. The aqueous phase extracted with DCM (10 mL), the combined organic phases were washed with sat. aqueous NaHCO₃, washed with brine, dried with magnesium sulfate, filtered and evaporated under reduced pressure. The residue was purified by chromatography on silica gel (0-40% EtOAc in heptanes) to afford methyl 3-(difluoromethyl)-2-methylbenzoate (83 mg, 30%).

A third intermediate, methyl 2-(bromomethyl)-3-(difluoromethyl)benzoate, was made as follows. To a solution of methyl 3-(difluoromethyl)-2-methyl-benzoate (80 mg, 0.400 mmol) in carbon tetrachloride (2.0 mL) was added N-bromosuccinimide (78 mg, 0.440 mmol). The reaction was heated to 85° C. for 5 minutes before AIBN (20 mg, 0.120 mmol) was added. The reaction was stirred at 85° C. for 4 h and was concentrated under reduced pressure. The residue was purified by chromatography on silica gel (0-5% EtOAc in heptanes) to afford methyl 2-(bromomethyl)-3-(difluoromethyl)benzoate (97 mg, 87%).

Finally, to form Compound 59, a mixture of 3-[3-[(R)-fluoro-(4-methyl-1,2,4-triazol-3-yl)methyl]oxetan-3-yl]aniline (synthesized as in WO2019/148005, 90 mg, 0.343 mmol) and methyl 2-(bromomethyl)-3-(difluoromethyl)benzoate (96 mg, 0.343 mmol) in acetonitrile (2.3 mL) and water (1.1 mL) was cooled to 0° C., then a solution of silver nitrate (76 mg, 0.446 mmol) in water (0.5 mL) was added drop-wise. The reaction was stirred for 48 h at rt. A saturated solution of sodium bicarbonate was added until the pH reached −8 and the reaction was diluted with 4:1 CHCl₃/IPA mixture (10 mL). The layers were separated, the aqueous phase extracted with 4:1 CHCl₃/IPA mixture (3×10 mL), and the combined organic phases were dried with magnesium sulfate, filtered and evaporated under reduced pressure. The residue was purified by chromatography on C18 silica gel (15-50% acetonitrile in 10 mM ammonium bicarbonate, pH=10) to afford (R)-4-(difluoromethyl)-2-(3-(3-(fluoro(4-methyl-4H-1,2,4-triazol-3-yl)methyl)oxetan-3-yl)phenyl)isoindolin-1-one (30 mg, 20%), (59). ¹H NMR (400 MHz, DMSO-d6) δ 8.35 (s, 1H), 7.93 (t, J=8.4 Hz, 2H), 7.87 (d, J=7.4 Hz, 1H), 7.71 (t, J=7.6 Hz, 1H), 7.58 (s, 1H), 7.39-7.34 (m, 1H), 7.29 (t, J=55.0 Hz, 1H), 6.95 (d, J=7.7 Hz, 1H), 6.28 (d, J=45.9 Hz, 1H), 5.37 (d, J=6.6 Hz, 1H), 5.21 (d, J=6.0 Hz, 1H), 5.16-5.01 (m, 3H), 4.83 (dd, J=5.7, 4.2 Hz, 1H), 3.19 (s, 3H). LCMS [M+H]⁺ or [M−H]⁻=429.2.

### Example 44: Compound 187

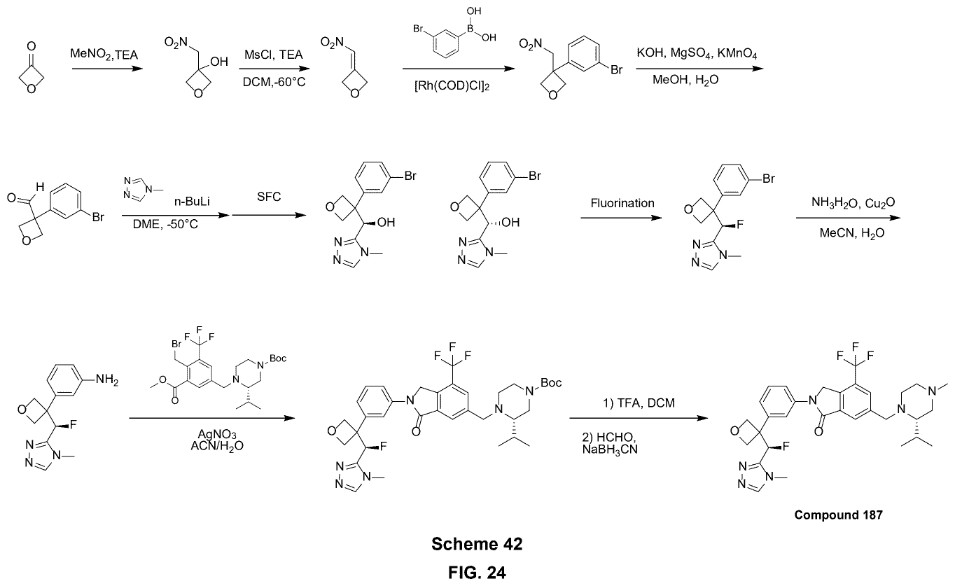

Compound 187 can be synthesized according to Scheme 42, FIG. 24.

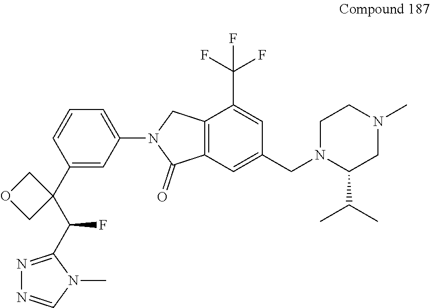 [SMILES: CC(C)[C@H]1CN(C)CCN1Cc1cc2c(c(C(F)(F)F)c1)CN(c1cccc(C3([C@@H](F)c4nncn4C)COC3)c1)C2=O]

A first intermediate, 3-(nitromethyl)oxetan-3-ol, is synthesized as follows. To a mixture of oxetan-3-one (100.0 g, 1.39 mol) in nitromethane (105.2 mL, 1.94 mol) was added triethylamine (38.6 mL, 277.5 mmol) at 0° C., the mixture was stirred at 25° C. for 16 h. The mixture was concentrated and the residue was purified by silica gel chromatography (mobile phase: ethyl acetate/petroleum ether, gradient 0% to 25%) to afford 3-(nitromethyl)oxetan-3-ol (130 g, 70% yield) as a yellow oil. ¹H NMR (400 MHz, CDCl₃): δ 4.81 (s, 2H), 4.71 (d, J=7.6 Hz, 2H), 4.65 (d, J=7.6 Hz, 2H).

A second intermediate, 3-(nitromethylene)oxetane was synthesized as follows. To a mixture of the first intermediate, 3-(nitromethyl)oxetan-3-ol (30.0 g, 225.4 mmol), in dichloromethane (500 mL) was added methanesulfonyl chloride (42.5 mL, 548.4 mmol) at −60° C., followed by triethylamine (125.7 mL, 901.6 mmol), the mixture was stirred for 4 h. The mixture was poured into saturated NH₄Cl aqueous (300 mL) and extracted with chloromethane (3×200 mL). The organic layer was combined and concentrated under reduced pressure. The residue was purified by silica gel chromatography (mobile phase: ethyl acetate/petroleum ether, gradient 0% to 15%) to afford 3-(nitromethylene)oxetane (22 g, 85% yield) as a yellow solid.

A third intermediate, 3-(3-bromophenyl)-3-(nitromethyl)oxetane, was synthesized as follows. Under N₂ atmosphere, potassium hydroxide aqueous solution (1.5 M, 28.7 mL, 43.4 mmol) was added to chloro(1,5-cyclooctadiene)rhodium(I)dimer (1.07 g, 2.17 mmol; CAS 12092-47-6) in flask dropwise. The mixture was stirred at 25° C. for 30 min. Then a solution of 3-bromophenylboronic acid (13.09 g, 65.1 mmol, 1.5 eq.) in 1,4-dioxane (150 ml) was added at 0-10° C. over 10 min, followed by the addition of 3-(nitromethylene)oxetane (5 g, 43.4 mmol) in 1,4-dioxane (10 ml) dropwise. After stirring for 30 min, another portion of 3-bromophenylboronic acid (13.09 g, 65.1 mmol, 1.5 eq.) was added. The reaction mixture was stirred at 25° C. for 16 h. The reaction was diluted with water (150 mL) and extracted with ethyl acetate (3×100 mL). The organic layers were combined, dried over anhydrous sodium sulfate and concentrated under vacuum. The residue was purified by silica gel chromatography (mobile phase: ethyl acetate/petroleum ether, gradient 0% to 25%) to afford 3-(3-bromophenyl)-3-(nitromethyl)oxetane (10.6 g, 89.7% yield) as yellow solid. ¹H NMR (400 MHz, CDCl₃): δ 7.44-7.38 (m, 1H), 7.26-7.23 (m, 2H), 7.04-7.02 (m, 1H), 5.05 (d, J=7.2 Hz, 2H), 5.01 (s, 2H), 4.89 (d, J=6.8 Hz, 2H).

A fourth intermediate, 3-(3-bromophenyl)oxetane-3-carbaldehyde, was synthesized as follows. 1 M Potassium hydroxide in methanol (4.04 mL, 4.04 mmol) was added dropwise over 15 min to 3-(3-bromophenyl)-3-(nitromethyl)oxetane (1.0 g, 3.68 mmol) in methanol (15 mL) at 0° C. The resulting mixture was stirred for 15 min, then a solution of potassium permanganate (638.9 mg, 4.04 mmol) and magnesium sulfate (398.1 mg, 3.31 mmol) in water (3 mL) was added dropwise over 15 min. The resulting mixture was warmed to ambient temperature and stirred for 1h. 2-methoxy-2-methylpropane (15 mL) was added, the solution was filtered through a pad of Celite. The filtrate was concentrated to remove most of the solvent, then 20 mL water was added. The solution was extracted with ethyl acetate (3×25 mL). The combined organic phases were dried over anhydrous sodium sulfate and concentrated. The residue was purified by silica gel chromatography (mobile phase: ethyl acetate/petroleum ether, gradient 0% to 30%) to afford 3-(3-bromophenyl)oxetane-3-carbaldehyde (700 mg, 79% yield) as a yellow solid. ¹H NMR (400 MHz, CDCl₃): δ 9.76 (s, 1H), 7.49-7.47 (m, 1H), 7.31-7.29 (m, 1H), 7.25-7.23 (m, 1H), 7.01-6.99 (m, 1H), 5.13 (d, J=6.4 Hz, 2H), 4.99 (d, J=6.4 Hz, 2H).

A fifth intermediate, (3-(3-bromophenyl)oxetan-3-yl)(4-methyl-4H-1,2,4-triazol-3-yl)methanol, was synthesized as follows. Under nitrogen protection, to a solution of 4-methyl-4H-1,2,4-triazole (155.1 mg, 1.9 mmol) in 1,2-dimethoxyethane (10 mL) was added n-butyllithium (0.75 mL, 1.9 mmol, 2.5M in hexane) at −50° C. The resulting mixture was stirred for 1 h at −50° C. Then a solution of 3-(3-bromophenyl)oxetane-3-carbaldehyde (300.0 mg, 1.24 mmol) in 1,2-dimethoxyethane (1 mL) was added dropwise. The reaction mixture was warmed to 0° C. slowly and stirred for another 1 h. The mixture was quenched with water (30 mL) and extracted with dichloromethane (3×10 mL). The combined organic layers were dried over anhydrous sodium sulfate and concentrated. The residue was purified by silica gel chromatography (mobile phase: methanol/dichloromethane, gradient 0% to 10%) to afford (3-(3-bromophenyl)oxetan-3-yl)(4-methyl-4H-1,2,4-triazol-3-yl)-methanol (330 mg, 81.8% yield) as a white solid. ¹H NMR (400 MHz, DMSO-d₆): δ 8.23 (s, 1H), 7.43-7.41 (m, 1H), 7.24-7.20 (m, 1H), 7.14 (d, J=1.6 Hz, 1H), 6.96 (d, J=7.6 Hz, 1H), 5.31 (s. 1H), 5.14-5.11 (m, 1H), 5.09-5.08 (m, 1H), 4.88 (d, J=6.4 Hz, 1H), 4.68-4.65 (m, 1H), 3.04 (s, 3H).

A pair of sixth intermediates, enantiomers (R)-(3-(3-bromophenyl)oxetan-3-yl)(4-methyl-4H-1,2,4-triazol-3-yl)methanol and (S)-(3-(3-bromophenyl)oxetan-3-yl)(4-methyl-4H-1,2,4-triazol-3-yl)methanol, were obtained as follows. (3-(3-bromophenyl)oxetan-3-yl)(4-methyl-4H-1,2,4-triazol-3-yl)methanol (60 g, 203.6 mmol) was further purified by chiral SFC (Column=Daicel Chiralpak AD; column dimensions=250 mm×50 mm×10 μm; detection wavelength=220 nm, flow rate=200 mL/min; run time=6 min; column temperature=40° C.) with 0.1% ammonium hydroxide-45% ethanol-carbon dioxide) to afford: (R)-(3-(3-bromophenyl)oxetan-3-yl)(4-methyl-4H-1,2,4-triazol-3-yl)methanol (Peak 1, retention time=3.546 min) (27 g, 41% yield) as a white solid and (S)-(3-(3-bromophenyl)oxetan-3-yl)(4-methyl-4H-1,2,4-triazol-3-yl)methanol (Peak 2, retention time=4.351 min) (27.9 g, 42.4% yield) as a white solid.

A seventh intermediate, (R)-3-((3-(3-bromophenyl)oxetan-3-yl)fluoromethyl)-4-methyl-4H-1,2,4-triazole, was synthesized according to two possible methods. In a first method (“A”), to a solution of (R)-(3-(3-bromophenyl)oxetan-3-yl)(4-methyl-4H-1,2,4-triazol-3-yl)methanol (27 g, 83.3 mmol) in dichloromethane (750 mL) was added diethylaminosulfur trifluoride (16.51 mL, 124.9 mmol) at −78° C. The reaction mixture was stirred for 16 h. The reaction was quenched with saturated ammonium chloride solution (500 mL) and diluted with dichloromethane (1000 mL). The separated organic phase was washed with brine (2×300 mL), dried over anhydrous sodium sulfate and concentrated to dryness. The crude was purified by silica gel chromatography (mobile phase: methanol/dichloromethane, gradient 0% to 6%) to give (R)-3-((3-(3-bromophenyl)oxetan-3-yl)fluoromethyl)-4-methyl-4H-1,2,4-triazole (23 g, 84.7% yield) as a white solid. LCMS [M+H]⁺=325.9 and 327.6.

In a second method (“B”), to a solution of (S)-[3-(3-bromophenyl)oxetan-3-yl]-(4-methyl-1,2,4-triazol-3-yl)methanol (11.0 g, 33.9 mmol) in toluene (300 mL) and acetonitrile (100 mL) was added pyridine-2-sulfonyl fluoride (6.02 g, 37.3 mmol) at −10° C., followed by 1,8-diazabicyclo[5.4.0]undec-7-ene (10.2 mL, 67.9 mmol). The result mixture was stirred at 15° C. for 16 h. The reaction mixture was diluted with water (500 mL) and extracted with ethyl acetate (3×150 mL). The combined organic layers were washed with brine (100 mL), dried over anhydrous sodium sulfate and concentrated under reduced pressure. The residue was purified by silica gel chromatography (mobile phase: methanol/dichloromethane, gradient 0% to 10%) afford (R)-3-((3-(3-bromophenyl)oxetan-3-yl)fluoromethyl)-4-methyl-4H-1,2,4-triazole (9 g, 81.3% yield) as a yellow solid. LCMS: [M+H]⁺=326.1 and 328.1.

¹H NMR (400 MHz, methanol-d₄): δ 8.32 (s, 1H), 7.47-7.45 (m, 1H), 7.26-7.22 (s, 2H), 7.04 (d, J=8.0 Hz, 1H), 6.26 (d, J=45.6 Hz, 1H), 5.41 (d, J=6.8 Hz, 1H), 5.26 (d, J=6.4 Hz, 1H), 5.15-5.13 (m, 1H), 4.95-4.92 (m, 1H), 3.19 (s, 3H).

An eighth intermediate, (R)-3-(3-(fluoro(4-methyl-4H-1,2,4-triazol-3-yl)methyl)oxetan-3-yl)aniline, to a sealed tube was added (R)-3-((3-(3-bromophenyl)oxetan-3-yl)fluoromethyl)-4-methyl-4H-1,2,4-triazole (15 g, 46.0 mmol), copper(I) oxide (3.29 g, 23.0 mmol), ammonia hydroxide (98.3 mL, 919.8 mmol) and acetonitrile (78 mL). The mixture was stirred at 100° C. for 16 h. The reaction mixture was filtered and concentrated reduced pressure. The residue was diluted with water (100 mL), then extracted with dichloromethane (5×100 mL). The organic layer dried over anhydrous sodium sulfate, filtered and concentrated under reduced pressure to afford (R)-3-(3-(fluoro(4-methyl-4H-1,2,4-triazol-3-yl)methyl)oxetan-3-yl)aniline (10.0 g, 83% yield) as a yellow solid which was used directly for next step. LCMS: [M+H]⁺=263.1.

A ninth intermediate, tert-butyl (S)-4-(4-(bromomethyl)-3-(methoxycarbonyl)-5-(trifluoromethyl)benzyl)-3-isopropylpiperazine-1-carboxylate, was prepared as follows. To a solution of tert-butyl (S)-3-isopropylpiperazine-1-carboxylate (13.1 g, 57.5 mmol) in 1,2-dichloroethane (250 mL) was added acetic acid (6.0 mL, 104.6 mmol) and stirred for 10 mins. Then methyl 2-(bromomethyl)-5-formyl-3-(trifluoromethyl)benzoate (17.0 g, 52.3 mmol) and sodium triacetoxyborohydride (33.3 g, 156.9 mmol) was added. The mixture was stirred at 25° C. for 16 hours. The reaction mixture was diluted with water (100 mL) and extracted with dichloromethane (3×100 mL). The combined organic layers were dried over anhydrous sodium sulfate and concentrated under reduced pressure. The residue was purified by silica gel chromatography (mobile phase: ethyl acetate/petroleum ether, gradient 0% to 20%) afford tert-butyl (S)-4-(4-(bromomethyl)-3-(methoxycarbonyl)-5-(trifluoromethyl)benzyl)-3-isopropylpiperazine-1-carboxylate (16 g, 56.9% yield) as yellow oil. LCMS: [M+H]⁺=537.2.

A tenth intermediate 10, tert-butyl (S)-4-((2-(3-(3-((R)-fluoro(4-methyl-4H-1,2,4-triazol-3-yl)methyl)oxetan-3-yl)phenyl)-3-oxo-7-(trifluoromethyl)isoindolin-5-yl)methyl)-3-isopropylpiperazine-1-carboxylate, was prepared as follows. To a mixture of (R)-3-(3-(fluoro(4-methyl-4H-1,2,4-triazol-3-yl)methyl)oxetan-3-yl)aniline (7.0 g, 26.7 mmol) and tert-butyl (S)-4-(4-(bromomethyl)-3-(methoxycarbonyl)-5-(trifluoromethyl)benzyl)-3-isopropylpiperazine-1-carboxylate (15.8 g, 29.4 mmol) in acetonitrile (300 mL) was added a solution of silver nitrate (5.9 g, 34.7 mmol) in water (100 mL) at 0° C. The mixture was stirred at 15° C. for 16 h then filtered and the filtrate was concentrated to remove most of solvent. The aqueous residue was extracted with dichloromethane (3×100 mL). The combined organic layers were dried over sodium sulfate and concentrated under reduced pressure. The residue was purified by silica gel chromatography (mobile phase: methanol/dichloromethane, gradient 0% to 5%) afford (S)-tert-butyl 4-((2-(3-(3-((R)-fluoro(4-methyl-4H-1,2,4-triazol-3-yl)methyl)oxetan-3-yl)phenyl)-3-oxo-7-(trifluoromethyl)isoindolin-5-yl)methyl)-3-isopropylpiperazine-1-carboxylate (9.8 g, 53.5% yield) as a yellow solid.

Finally, Compound 187, 2-(3-(3-((R)-fluoro(4-methyl-4H-1,2,4-triazol-3-yl)methyl)oxetan-3-yl)phenyl)-6-(((S)-2-isopropyl-4-methylpiperazin-1-yl)methyl)-4-(trifluoromethyl)isoindolin-1-one, was obtained as follows. To a solution of tert-butyl (S)-4-((2-(3-(3-((R)-fluoro(4-methyl-4H-1,2,4-triazol-3-yl)methyl)oxetan-3-yl)phenyl)-3-oxo-7-(trifluoromethyl)isoindolin-5-yl)methyl)-3-isopropylpiperazine-1-carboxylate (1 g, 1.5 mmol) in dichloromethane (30 mL) was added trifluoroacetic acid (1.5 mL, 19.1 mmol) at 25° C. The mixture was stirred at 15° C. for 3 h, then the mixture was concentrated under reduced pressure to afford 2-(3-(3-((R)-fluoro(4-methyl-4H-1,2,4-triazol-3-yl)methyl)oxetan-3-yl)phenyl)-6-(((S)-2-isopropylpiperazin-1-yl)methyl)-4(trifluoromethyl)isoindolin-1-one as crude product which was used directly. The above product was dissolved with methanol (20 mL), then 30% aqueous formaldehyde (1.7 mL, 22.4 mmol) and sodium cyanoborohydride (276.2 mg, 4.4 mmol) were added. The reaction mixture was stirred at 15° C. for 3 hours. The mixture was concentrated under reduced pressure. The residue purified by silica gel chromatography (mobile phase: methanol/dichloromethane, gradient 0% to 10%) afford 2-(3-(3-((R)-fluoro(4-methyl-4H-1,2,4-triazol-3-yl)methyl)oxetan-3-yl)phenyl)-6-(((S)-2-isopropyl-4-methylpiperazin-1-yl)methyl)-4-(trifluoromethyl)isoindolin-1-one (800 mg, 93.7% yield) as a white solid. ¹H NMR (400 MHz, CDCl₃): δ 8.08 (s, 1H), 7.90 (s, 1H), 7.84 (s, 1H), 7.64 (d, J=8.0 Hz, 1H), 7.49 (s, 1H), 7.38 (t, J=8.0 Hz, 1H), 6.77 (d, J=7.6 Hz, 1H), 6.47 (d, J=46 Hz, 1H), 5.37-5.20 (m, 3H), 5.04-4.84 (m, 3H), 4.27 (d, J=14.0 Hz, 1H), 3.24 (d, J=13.6 Hz, 1H), 3.03 (s, 3H), 2.78-2.65 (m, 2H), 2.61 (d, J=11.2 Hz, 1H), 2.39-2.21 (m, 6H), 2.17-1.93 (m, 1H), 1.00 (d, J=6.8 Hz, 3H), 0.97 (d, J=6.8 Hz, 3H).

### Example 45: Compound 232

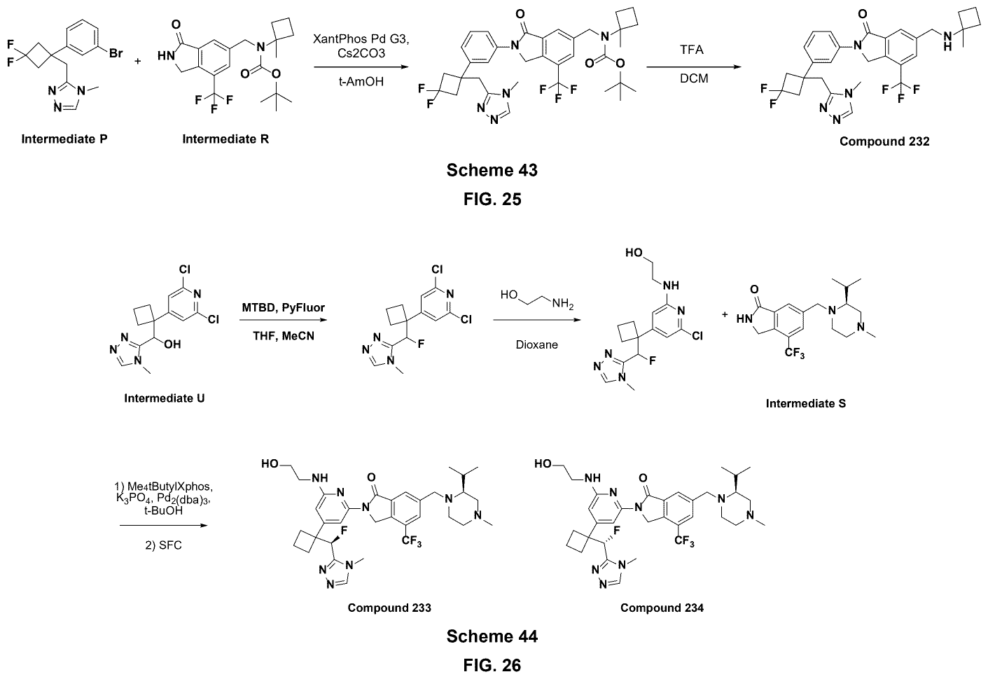

Compound 232 ((2-(3-(3,3-difluoro-1-((4-methyl-4H-1,2,4-triazol-3-yl)methyl)-cyclobutyl)phenyl)-6-(((1-methylcyclobutyl)amino)methyl)-4-(trifluoromethyl)isoindolin-1-one)) can be synthesized according to Scheme 43, FIG. 25.

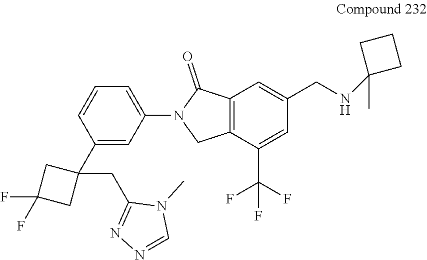 [SMILES: Cn1cnnc1CC1(c2cccc(N3Cc4c(cc(CNC5(C)CCC5)cc4C(F)(F)F)C3=O)c2)CC(F)(F)C1]

A pressure flask equipped with a stir bar was charged with 3-((1-(3-bromophenyl)-3,3-difluorocyclobutyl)methyl)-4-methyl-4H-1,2,4-triazole (Intermediate P; 585.5 mg, 1.71 mmol), tert-butyl (1-methylcyclobutyl)((3-oxo-7-(trifluoromethyl)isoindolin-5-yl)methyl)-carbamate (Intermediate R; 50 mg, 1.88 mmol), XantPhos Pd G3 (81.2 mg, 0.09 mmol) and cesium carbonate (1.672 g, 5.13 mmol). The flask was flushed with nitrogen for several minutes and degassed tert-amyl alcohol (17.1 mL) was added via syringe. The flask was sealed and the reaction mixture was stirred at 120° C. for 15 h. The reaction was cooled down to rt, silica gel was added and the solvent was evaporated. The crude product was purified by chromatography on silica gel (0-8% of MeOH in DCM) to afford a first intermediate, tert-butyl ((2-(3-(3,3-difluoro-1-((4-methyl-4H-1,2,4-triazol-3-yl)methyl)cyclobutyl)phenyl)-3-oxo-7-(trifluoromethyl)isoindolin-5-yl)methyl)(1-methylcyclobutyl)carbamate (1.118 g, 99% yield). LCMS (ESI) m/z: 660.6 [M+H]⁺

Trifluoroacetic acid (8.5 mL) was added to a solution of the first intermediate (1.118 g, 1.69 mmol) in DCM (8.5 mL) and the solution was stirred at rt for 30 minutes. The reaction mixture was diluted with toluene and concentrated to dryness. The crude residue was purified by chromatography on C18 silica gel (15-40% acetonitrile in formic acid (0.5% aqueous). The cleanest fractions were combined and concentrated, then basified with 1 N NaOH solution and extracted with CHCl₃: IPA (5×). The organics were combined, dried over sodium sulfate, filtered and evaporated to dryness, then dissolved in ACN/water and lyophilized to obtain 2-(3-(3,3-difluoro-1-((4-methyl-4H-1,2,4-triazol-3-yl)methyl)cyclobutyl)-phenyl)-6-(((1-methylcyclobutyl)amino)methyl)-4-(trifluoromethyl)isoindolin-1-one (634.2 mg, 67% yield). LCMS (ESI) m/z: 560.3 [M+H]⁺¹H NMR (400 MHz, DMSO-d6) δ 8.17 (s, 1H), 8.05 (s, 1H), 8.01 (s, 1H), 7.92 (dd, J=8.2, 1.3 Hz, 1H), 7.39 (t, J=1.7 Hz, 1H), 7.34 (t, J=8.0 Hz, 1H), 6.78 (d, J=8.2 Hz, 1H), 5.09 (s, 2H), 3.82 (s, 2H), 3.31-3.20 (m, 4H), 3.02 (q, J=14.1 Hz, 2H), 2.72 (s, 3H), 2.03-1.91 (m, 2H), 1.77-1.61 (m, 4H), 1.23 (s, 3H).

### Example 46: Compounds 233 and Compound 234

Compounds 233 and 234 (2-(4-(1-((R)-fluoro(4-methyl-4H-1,2,4-triazol-3-yl)methyl)cyclobutyl)-6-((2-hydroxyethyl)amino)pyridin-2-yl)-6-(((S)-2-isopropyl-4-methyl-piperazin-1-yl)methyl)-4-(trifluoromethyl)isoindolin-1-one and 2-(4-(1-((S)-fluoro(4-methyl-4H-1,2,4-triazol-3-yl)methyl)cyclobutyl)-6-((2-hydroxyethyl)amino)pyridin-2-yl)-6-(((S)-2-isopropyl-4-methylpiperazin-1-yl)methyl)-4-(trifluoromethyl)isoindolin-1-one) can be synthesized according to Scheme 44, FIG. 26.

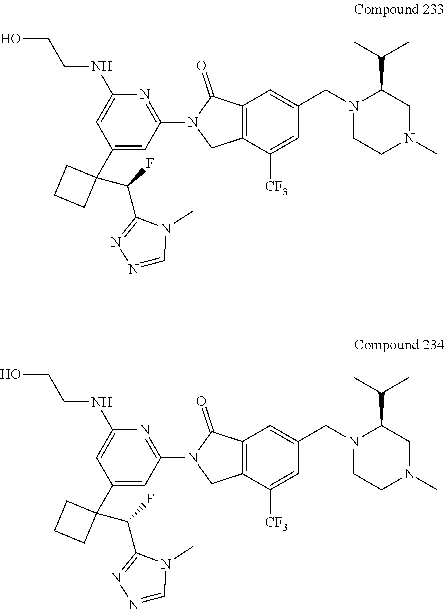 [SMILES: CC(C)[C@H]1CN(C)CCN1Cc1cc2c(c(C(F)(F)F)c1)CN(c1cc(C3([C@@H](F)c4nncn4C)CCC3)cc(NCCO)n1)C2=O ; CC(C)[C@H]1CN(C)CCN1Cc1cc2c(c(C(F)(F)F)c1)CN(c1cc(C3([C@H](F)c4nncn4C)CCC3)cc(NCCO)n1)C2=O]

A first intermediate, 2,6-dichloro-4-(1-(fluoro(4-methyl-4H-1,2,4-triazol-3-yl)methyl)cyclobutyl)pyridine, can be obtained from Intermediate U, as follows. To a stirred solution (1-(2,6-dichloropyridin-4-yl)cyclobutyl)(4-methyl-4H-1,2,4-triazol-3-yl)methanol (800 mg, 2.55 mmol) in THF (7 mL) and MeCN (7 mL) was added 1-methyl-2,3,4,6,7,8-hexa-hydropyrimido[1,2-a]pyrimidine (0.73 mL, 5.11 mmol) followed by 2-pyridinesulfonyl fluoride (0.43 mL, 2.81 mmol) at rt. The resulting reaction mixture was stirred at 25° C. for 20 h. The reaction was diluted with water (10 ml) extracted with DCM (3×50 ml). Combined organic layers were dried over sodium sulfate, filtered and concentrated. The crude product was purified by chromatography on silica gel (0-5% of MeOH in DCM) to get 2,6-dichloro-4-(1-(fluoro(4-methyl-4H-1,2,4-triazol-3-yl)methyl)cyclobutyl)pyridine (605 mg, 75% yield) as a white solid. LCMS (ESI) m/z: 315.1, 317.0 [M+H]⁺

A second intermediate, 2-((6-chloro-4-(1-(fluoro(4-methyl-4H-1,2,4-triazol-3-yl)methyl)cyclobutyl)pyridin-2-yl)amino)ethanol, can be obtained as follows. In a sealed tube and to a stirred solution 2,6-dichloro-4-(1-(fluoro(4-methyl-4H-1,2,4-triazol-3-yl)methyl)cyclobutyl)pyridine (64 mg, 0.2 mmol) in 1,4-dioxane (0.41 mL) was added ethanolamine (0.25 mL, 4.06 mmol). The resulting reaction mixture was stirred at 80° C. for 72 h. The reaction was concentrated and the crude product was purified by chromatography on silica gel (0-5% of MeOH in DCM) to afford 2-((6-chloro-4-(1-(fluoro(4-methyl-4H-1,2,4-triazol-3-yl)methyl)cyclobutyl)pyridin-2-yl)amino)ethanol (60 mg, 87% yield) as a white solid. LCMS (ESI) m/z: 340.2, 342.2 [M+H]⁺

A microwave vial was charged with 2-((6-chloro-4-(1-(fluoro(4-methyl-4H-1,2,4-triazol-3-yl)methyl)cyclobutyl)pyridin-2-yl)amino)ethanol (60 mg, 0.18 mmol), (S)-6-((2-isopropyl-4-methylpiperazin-1-yl)methyl)-4-(trifluoromethyl)isoindolin-1-one (Intermediate S; 69 mg, 0.19 mmol), Me₄tButylXphos (8.5 mg, 0.02 mmol), Pd₂(dba)₃ (8.1 mg, 0.01 mmol) and K₃PO₄ (112.4 mg, 0.53 mmol). The vial was purged with nitrogen before degassed anhydrous tert-butanol (0.88 mL) was added and the vial was sealed. The reaction mixture was stirred at 110° C. for 15 h. The volatiles were evaporated and the residue was purified by chromatography on C18 silica gel (0-100% acetonitrile in ammonium formate buffer, pH=3.8). Appropriate fractions were concentrated, frozen and lyophilized to give the racemic mixture (50 mg, 43% yield) as a white solid.

The above racemate was further purified by chiral SFC (Column=i-Amylose-1; Column dimensions=250 mm×10 mm×5 μm; Detection wavelength=310 nm; Flow rate=10 mL/min; Run time=15 min; Column temperature=40° C.) with 0.1% ammonium hydroxide −40% IPA/carbon dioxide) to afford Example 46: Compounds 33 and Compound 234).

2-(4-(1-((R)-fluoro(4-methyl-4H-1,2,4-triazol-3-yl)methyl)cyclobutyl)-6-((2-hydroxyethyl)amino)pyridin-2-yl)-6-(((S)-2-isopropyl-4-methylpiperazin-1-yl)methyl)-4-(trifluoromethyl)isoindolin-1-one one (Peak 1) (18.4 mg, 16% yield). LCMS (ESI) m/z: 659.2 [M+H]⁺¹H NMR (400 MHz, DMSO-d6) δ 8.30 (s, 1H), 7.95 (s, 1H), 7.89 (s, 1H), 7.41 (s, 1H), 6.52 (t, J=5.5 Hz, 1H), 6.04 (d, J=44.8 Hz, 1H), 5.97 (s, 1H), 5.10 (s, 2H), 4.61 (s, 1H), 4.18 (d, J=14.5 Hz, 1H), 3.51 (s, 2H), 3.35-3.25 (m, 4H), 3.15 (s, 3H), 2.83-2.54 (m, 4H), 2.45-2.37 (m, 1H), 2.32-2.15 (m, 4H), 2.12 (s, 3H), 2.08-1.77 (m, 4H), 0.91 (d, J=6.7 Hz, 3H), 0.86 (d, J=6.6 Hz, 3H).

2-(4-(1-((S)-fluoro(4-methyl-4H-1,2,4-triazol-3-yl)methyl)cyclobutyl)-6-((2-hydroxyethyl)amino)pyridin-2-yl)-6-(((S)-2-isopropyl-4-methylpiperazin-1-yl)methyl)-4-(trifluoromethyl)isoindolin-1-one (peak 2) (18.1 mg, 16% yield). LCMS (ESI) m/z: 659.2 [M+H]⁺¹H NMR (400 MHz, DMSO-d6) δ 8.27 (s, 1H), 7.95 (s, 1H), 7.89 (s, 1H), 7.41 (s, 1H), 6.52 (t, J=5.5 Hz, 1H), 6.04 (d, J=44.8 Hz, 1H), 5.97 (s, 1H), 5.08 (s, 2H), 4.62 (s, 1H), 4.18 (d, J=14.4 Hz, 1H), 3.51 (t, J=6.0 Hz, 2H), 3.35-3.20 (m, 2H), 3.15 (s, 3H), 2.83-2.54 (m, 4H), 2.45-2.37 (m, 1H), 2.32-2.15 (m, 4H), 2.12 (s, 3H), 2.09-1.76 (m, 4H), 0.91 (d, J=6.7 Hz, 3H), 0.86 (d, J=6.6 Hz, 3H).

### Example 47: Compound 235

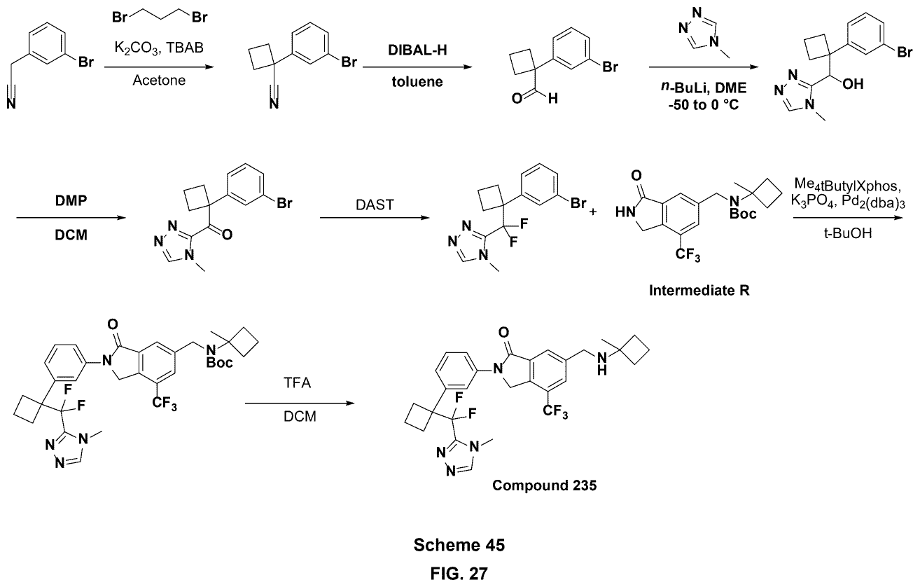

Compound 235 (2-(3-(1-(difluoro(4-methyl-4H-1,2,4-triazol-3-yl)methyl)-cyclobutyl)phenyl)-6-(((1-methylcyclobutyl)amino)methyl)-4-(trifluoromethyl)isoindolin-1-one) can be synthesized according to Scheme 45, FIG. 27.

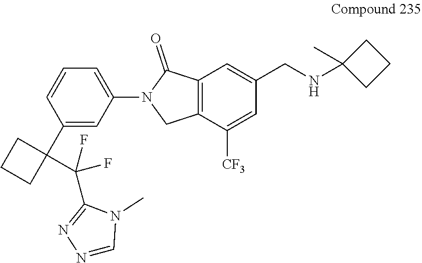 [SMILES: Cn1cnnc1C(F)(F)C1(c2cccc(N3Cc4c(cc(CNC5(C)CCC5)cc4C(F)(F)F)C3=O)c2)CCC1]

A first intermediate, 1-(3-bromophenyl)cyclobutanecarbonitrile, can be made as follows. To a solution of 2-(3-bromophenyl)acetonitrile (1 g, 5.1 mmol) and 1,3-dibromopropane (0.62 mL, 6.12 mmol) in acetone (11.3 mL) were added potassium carbonate (1.76 g, 12.75 mmol) and tetrabutylammonium bromide (164.4 mg, 0.510 mmol). The resulting suspension was heated at 55° C. for 5 days. The reaction mixture was cooled to rt and filtered through a pad of Celite. The pad was rinsed with acetone and the filtrate was concentrated to dryness. The crude product was purified by chromatography on silica gel (0-35% of EtAOc in heptanes) to afford 1-(3-bromophenyl)cyclobutanecarbonitrile (530 mg, 44% yield). ¹H NMR (400 MHz, CDCl₃) δ 7.55 (t, J=1.9 Hz, 1H), 7.46 (ddt, J=7.7, 1.8, 0.9 Hz, 1H), 7.35 (ddt, J=7.9, 2.0, 1.0 Hz, 1H), 7.27 (t, J=7.8 Hz, 1H), 2.87-2.78 (m, 2H), 2.66-2.55 (m, 2H), 2.51-2.38 (m, 1H), 2.14-2.03 (m, 1H).

A second intermediate, 1-(3-bromophenyl)cyclobutanecarbaldehyde, can be made as follows. A 1 M DIBAL-H solution in toluene (2.5 mL, 2.47 mmol) was added slowly to a solution of 1-(3-bromophenyl)cyclobutanecarbonitrile (530 mg, 2.24 mmol) in toluene (13.2 mL) at −78° C. and the resulting mixture was stirred at that temperature for 1.5 h. The reaction was quenched by the addition of 1 N HCl solution, then warmed to rt and extracted with DCM (3×). The organics were combined, washed with 1 N HCl solution, washed with brine, dried over sodium sulfate, filtered and evaporated. The crude product was purified by chromatography on silica gel (0-50% of Et₂O in heptanes) to afford 1-(3-bromophenyl)cyclobutanecarbaldehyde (98.4 mg, 18% yield). ¹H NMR (400 MHz, CDCl₃) δ 9.53 (s, 1H), 7.41 (ddt, J=7.9, 1.8, 0.9 Hz, 1H), 7.30 (t, J=1.5 Hz, 1H), 7.24 (t, J=7.6 Hz, 1H), 7.07 (ddt, J=7.8, 1.8, 0.9 Hz, 1H), 2.77-2.68 (m, 2H), 2.39 (ddd, J=19.0, 9.4, 2.4 Hz, 2H), 2.09-1.88 (m, 2H).

A third intermediate, (1-(3-bromophenyl)cyclobutyl)(4-methyl-4H-1,2,4-triazol-3-yl)methanol, can be made as follows. 2.5 M n-BuLi solution in hexanes (0.2 mL, 0.49 mmol) was added to a solution of 4-methyl-1,2,4-triazole (41.03 mg, 0.49 mmol) in DME (4.1 mL) cooled to −50° C. and stirred at that temperature for 1h. 1-(3-bromophenyl)cyclobutane-carbaldehyde (98.4 mg, 0.41 mmol) was then added dropwise as a solution in DME (2 mL, then vial washed with 1 mL) and the reaction was allowed to slowly warm to rt. After 1 h, the reaction was quenched with saturated aqueous NH₄Cl solution, then most of the DME was evaporated. The resulting aqueous layer was extracted with a 4:1 mixture of CHCl₃: IPA (3×). The organics were combined, dried over sodium sulfate, filtered and evaporated. The crude mixture was purified by chromatography on silica gel (0-12% of MeOH in DCM) to afford (1-(3-bromophenyl)cyclobutyl)(4-methyl-4H-1,2,4-triazol-3-yl)methanol (49.7 mg, 37% yield). LCMS (ESI) m/z: 322.1, 324.1 [M+H]⁺

A fourth intermediate, (1-(3-bromophenyl)cyclobutyl)(4-methyl-4H-1,2,4-triazol-3-yl)methanone, can be made as follows. To a stirred solution of (1-(3-bromophenyl)cyclo-butyl)(4-methyl-4H-1,2,4-triazol-3-yl)methanol (49.7 mg, 0.15 mmol) in DCM (0.77 mL) was added Dess-Martin periodinane (130.9 mg, 0.31 mmol). The resulting reaction mixture was stirred at rt for 16 h. The reaction was then quenched with 10% aqueous Na₂S₂O₃ solution and saturated aqueous NaHCO₃ solution, stirred for 30 minutes and extracted with 4:1 mixture of CHCl₃: IPA (5×). The organics were combined, dried over sodium sulfate, filtered and evaporated to afford the crude (1-(3-bromophenyl)cyclobutyl)(4-methyl-4H-1,2,4-triazol-3-yl)methanone (53.9 mg, 109% yield), used as such in the next step. LCMS (ESI) m/z: 320.1, 322.0 [M+H]⁺.

A fifth intermediate, 3-((1-(3-bromophenyl)cyclobutyl)difluoromethyl)-4-methyl-4H-1,2,4-triazole, can be made as follows. A neat mixture of crude (1-(3-bromophenyl)cyclo-butyl)(4-methyl-4H-1,2,4-triazol-3-yl)methanone (33.9 mg, 0.11 mmol) and DAST (0.35 mL, 2.65 mmol) was stirred at 50° C. for 16 h. The reaction was not complete more DAST (0.35 mL, 2.65 mmol) was added and heated for another 17 h: The mixture was quenched by very slow addition of saturated aqueous NaHCO₃ solution, extracted with a 4:1 mixture of CHCl₃: IPA (3×). The organics were combined, dried over sodium sulfate, filtered and evaporated. The crude mixture was purified by chromatography on silica gel (50-100% of EtOAc in DCM) to afford 3-((1-(3-bromophenyl)cyclobutyl)difluoromethyl)-4-methyl-4H-1,2,4-triazole (17.8 mg, 49% yield). LCMS (ESI) m/z: 342.0, 344.0 [M+H]⁺.

A sixth intermediate, tert-butyl ((2-(3-(1-(difluoro(4-methyl-4H-1,2,4-triazol-3-yl)-methyl)cyclobutyl)phenyl)-3-oxo-7-(trifluoromethyl)isoindolin-5-yl)methyl)(1-methyl-cyclobutyl)carbamate, can be made as follows. A microwave vial equipped with a stir bar was charged with K₃PO₄ (22.1 mg, 0.1 mmol), fifth intermediate, 3-((1-(3-bromophenyl)-cyclobutyl)difluoromethyl)-4-methyl-4H-1,2,4-triazole (17.8 mg, 0.05 mmol), and tert-butyl (1-methylcyclobutyl)((3-oxo-7-(trifluoromethyl)isoindolin-5-yl)methyl)carbamate (Intermediate R; 22.8 mg, 0.06 mmol), Me₄tButylXphos (2.5 mg, 0.001 mmol) and tris(dibenzylideneacetone)dipalladium (0) (1.9 mg, 0.002 mmol). The vial was then flushed with nitrogen for several minutes and degassed tert-butanol (0.26 mL) was added via syringe. The vial was capped and the reaction mixture was stirred at 110° C. for 16 h then cooled down to rt. The reaction mixture was directly purified by chromatography on C18 silica gel (50-95% gradient of acetonitrile in ammonium formate buffer, pH=3.8). Appropriate fractions were concentrated, frozen and lyophilized to provide tert-butyl ((2-(3-(1-(difluoro(4-methyl-4H-1,2,4-triazol-3-yl)methyl)cyclobutyl)phenyl)-3-oxo-7-(trifluoromethyl)isoindolin-5-yl)methyl)(1-methylcyclobutyl)carbamate (10.2 mg, 30% yield). LCMS (ESI) m/z: 660.2 [M+H]⁺.

Trifluoroacetic acid (80 μL) was added to a solution of tert-butyl ((2-(3-(1-(difluoro(4-methyl-4H-1,2,4-triazol-3-yl)methyl)cyclobutyl)phenyl)-3-oxo-7-(trifluoromethyl)-isoindolin-5-yl)methyl)(1-methylcyclobutyl)carbamate (10.2 mg, 0.02 mmol) in DCM (80 μL) and the solution was stirred at rt for 2 h. Excess TFA was quenched by addition of saturated aqueous NaHCO₃ solution and the aqueous layer was extracted with a 4:1 mixture of CHCl₃: IPA (5×). The organics were combined, dried over sodium sulfate, filtered and evaporated. The crude mixture was purified by chromatography on C18 silica gel (20-55% gradient of acetonitrile in ammonium formate buffer, pH=3.8). Appropriate fractions were concentrated, frozen and lyophilized to provide 2-(3-(1-(difluoro(4-methyl-4H-1,2,4-triazol-3-yl)methyl)cyclobutyl)phenyl)-6-(((1-methylcyclobutyl)amino)methyl)-4-(trifluoro-methyl)isoindolin-1-one (Compound 235; 2.8 mg, 32% yield). LCMS (ESI) m/z: 560.1 [M+H]⁺¹H NMR (400 MHz, DMSO-d6) δ 8.37 (s, 1H), 8.30 (br s, 1H), 8.03 (s, 1H), 7.99 (s, 1H), 7.81 (d, J=8.0 Hz, 1H), 7.46 (s, 1H), 7.41-7.30 (m, 1H), 6.83 (d, J=7.2 Hz, 1H), 5.04 (s, 2H), 3.81 (s, 2H), 3.06-2.93 (m, 2H), 2.81 (s, 3H), 2.13-2.01 (m, 1H), 2.01-1.91 (m, 2H), 1.91-1.80 (m, 1H), 1.77-1.57 (m, 4H), 1.21 (s, 3H).

### Example 48: Compound 236

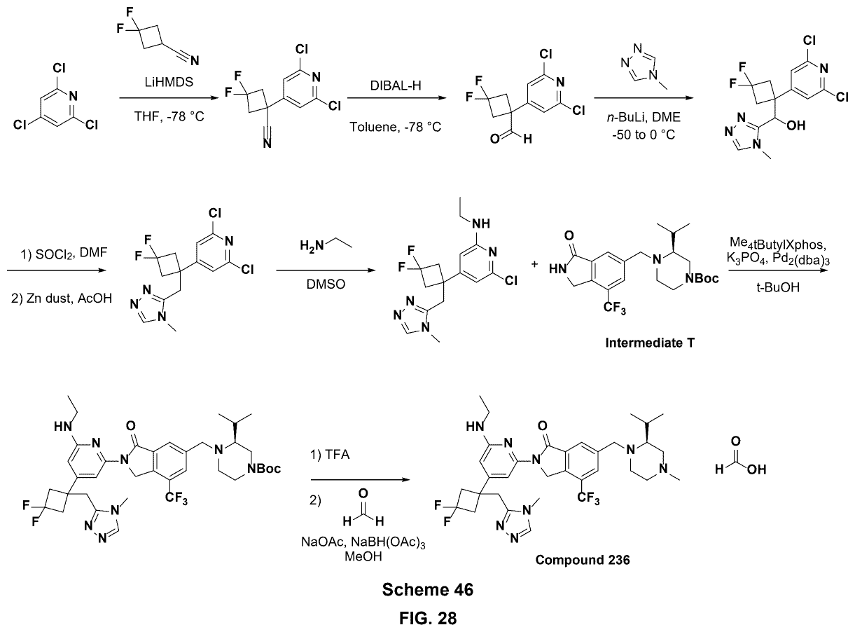

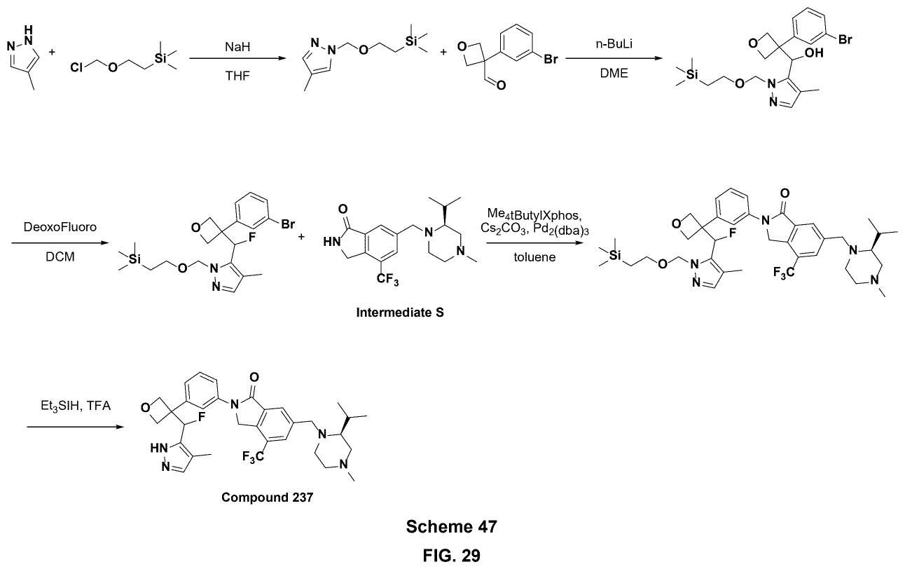

Compound 236 ((S)-2-(4-(3,3-difluoro-1-((4-methyl-4H-1,2,4-triazol-3-yl)methyl)-cyclobutyl)-6-(ethylamino)pyridin-2-yl)-6-((2-isopropyl-4-methylpiperazin-1-yl)methyl)-4-(trifluoromethyl)isoindolin-1-one formate) can be synthesized according to Scheme 46, FIG. 28.

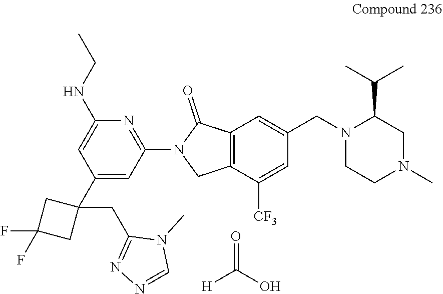

A first intermediate, 1-(2,6-dichloropyridin-4-yl)-3,3-difluorocyclobutanecarbonitrile, can be made as follows. 3,3-difluorocyclobutanecarbonitrile (673.9 mg, 5.76 mmol) and 2,4,6-trichlopyridine (1 g, 5.48 mmol) were dissolved in THF (20 mL) and cooled to −78° C. 1 M LiHMDS solution in THF (6.0 mL, 6.03 mmol) was added dropwise over 10 minutes. After stirring at −78 C for 10 minutes cooling bath was removed and reaction mixture was allowed to warm to rt and stirred for 2 h. The reaction was quenched by addition of saturated aqueous NH₄Cl solution and extracted with EtOAc (3×). Combined organic layers were washed with brine, dried over sodium sulfate, filtered and concentrated. The crude mixture was purified by chromatography on silica gel (0-50% of EtOAc in heptanes) to afford 1-(2,6-dichloropyridin-4-yl)-3,3-difluorocyclobutanecarbonitrile (900 mg, 62% yield). LCMS (ESI) m/z: 263.3 [M+H]⁺¹H NMR (400 MHz, CDCl₃) δ 7.40 s, 2H), 3.61-3.52 (m, 2H), 3.25-3.16 (m, 2H).

A second intermediate, 1-(2,6-dichloropyridin-4-yl)-3,3-difluorocyclobutanecarbaldehyde, was made as follows. 1 M DIBAL-H solution in heptane (6.8 mL, 6.84 mmol) was added slowly to a solution of 1-(2,6-dichloropyridin-4-yl)-3,3-difluorocyclobutanecarbonitrile (1.20 g, 4.56 mmol) in diethyl ether (18 mL) at −78° C. The resulting mixture was stirred for 2 h at −78° C. To the reaction mixture was added 5 g of Na₂SO₄ decahydrate and 50 mL of diethyl ether. The mixture was stirred at rt for 10 minutes and 1 M HCl solution (20 mL) was added. The mixture was stirred at rt for 10 minutes and organic layer was separated, dried over sodium sulfate, filtered and concentrated to give 1-(2,6-dichloropyridin-4-yl)-3,3-difluorocyclobutanecarbaldehyde (1.10 g, 91% yield). This crude was used without future purification.

A third intermediate, (1-(2,6-dichloropyridin-4-yl)-3,3-difluorocyclobutyl)(4-methyl-4H-1,2,4-triazol-3-yl)methanol, was made as follows. At −50° C., 2.5 M n-BuLi solution in hexanes (0.87 mL, 2.17 mmol) was added to a solution of 4-methyl-1,2,4-triazole (180 mg, 2.17 mmol) in DME (26 mL) and the reaction was stirred at that temperature for 1 h. Crude 1-(2,6-dichloropyridin-4-yl)-3,3-difluorocyclobutanecarbaldehyde (749.3 mg, 2.82 mmol) was then added dropwise as a solution in DME (10 mL) and the reaction was allowed to slowly warm to 0° C. (1 h). Once the reaction temperature reached to 0° C., it was stirred for additional 30 minutes after that it was quenched with saturated aqueous NH₄Cl solution and extracted with EtOAc (2×70 mL). The crude product was purified by chromatography on silica gel (0-20% of MeOH in DCM) to afford (1-(2,6-dichloropyridin-4-yl)-3,3-difluorocyclo-butyl)(4-methyl-4H-1,2,4-triazol-3-yl)methanol (145 mg, 19% yield).

A fourth intermediate, 2,6-dichloro-4-(3,3-difluoro-1-((4-methyl-4H-1,2,4-triazol-3-yl)methyl)cyclobutyl)pyridine, was made as follows. (1-(2,6-dichloropyridin-4-yl)-3,3-difluorocyclobutyl)(4-methyl-4H-1,2,4-triazol-3-yl)methanol (400 mg, 1.15 mmol) was dissolved in thionyl chloride (4.2 mL, 57.28 mmol) and to the solution was added DMF (10 μL). The solution was stirred at 55° C. for 20 minutes. The crude product was purified by chromatography on silica gel (0-20% of MeOH in DCM) to afford the chloro-intermediate, which was dissolved in acetic acid (5 mL). To the solution was added zinc (374.5 mg, 5.73 mmol) and stirred at 55° C. for 2 h. The reaction was filtered and concentrated. The crude product was purified by chromatography on silica gel (5-20% of MeOH in DCM) to afford 2,6-dichloro-4-(3,3-difluoro-1-((4-methyl-4H-1,2,4-triazol-3-yl)methyl)cyclobutyl)pyridine (321 mg, 84% yield). LCMS (ESI) m/z: 333.0, 335.0, 337.0 [M+H]⁺.

A fifth intermediate, 6-chloro-4-(3,3-difluoro-1-((4-methyl-4H-1,2,4-triazol-3-yl)methyl)cyclobutyl)-N-ethylpyridin-2-amine, was made as follows. In a microwave vial, 2,6-dichloro-4-(3,3-difluoro-1-((4-methyl-4H-1,2,4-triazol-3-yl)methyl)cyclobutyl)pyridine (100 mg, 0.3 mmol) was dissolved in DMSO (3 mL) and to the solution was added ethylamine (67% in water) (0.6 mL, 0.3 mmol). The resulting reaction mixture was stirred at 110° C. for 3 h then cooled to rt. The reaction was diluted with water (10 mL) and EtOAc (20 mL), phases were separated and the organic layer was washed with water (2×5 mL) and brine. The organic layer was dried over sodium sulfate, filtered and concentrated to give 6-chloro-4-(3,3-difluoro-1-((4-methyl-4H-1,2,4-triazol-3-yl)methyl)cyclobutyl)-N-ethylpyridin-2-amine (53 mg, 52% yield), which was used in the next step without purification. LCMS (ESI) m/z: 342.0, 344.0 [M+H]⁺.

A sixth intermediate, ((S)-tert-butyl 4-((2-(4-(3,3-difluoro-1-((4-methyl-4H-1,2,4-triazol-3-yl)methyl)cyclobutyl)-6-(ethylamino)pyridin-2-yl)-3-oxo-7-(trifluoromethyl)isoindolin-5-yl)methyl)-3-isopropylpiperazine-1-carboxylate, was made as follows. A microwave vial was charged with 6-chloro-4-(3,3-difluoro-1-((4-methyl-4H-1,2,4-triazol-3-yl)methyl)cyclo-butyl)-N-ethylpyridin-2-amine (54 mg, 0.16 mmol), (S)-tert-butyl 3-isopropyl-4-((3-oxo-7-(trifluoromethyl)isoindolin-5-yl)methyl)piperazine-1-carboxylate (Intermediate T; 76.7 mg, 0.17 mmol), Me₄tButylXphos (7.6 mg, 0.02 mmol), Pd₂(dba)₃ (7.2 mg, 0.01 mmol) and K₃PO₄ (100.6 mg, 0.47 mmol). The vial was purged with nitrogen before degassed anhydrous tert-butanol (0.93 mL) was added and the vial was sealed. The reaction mixture was stirred at 110° C. for 15 h. The reaction was concentrated and the crude product was purified by chromatography on silica gel (0-20% of MeOH in DCM (containing (0.3% of triethylamine)) to afford ((S)-tert-butyl 4-((2-(4-(3,3-difluoro-1-((4-methyl-4H-1,2,4-triazol-3-yl)methyl)cyclobutyl)-6-(ethylamino)pyridin-2-yl)-3-oxo-7-(trifluoromethyl)isoindolin-5-yl)methyl)-3-isopropylpiperazine-1-carboxylate as a complex with trimethylamine. LCMS (ESI) m/z: 747.4 [M+H]⁺.

To the crude ((S)-tert-butyl 4-((2-(4-(3,3-difluoro-1-((4-methyl-4H-1,2,4-triazol-3-yl)methyl)cyclobutyl)-6-(ethylamino)pyridin-2-yl)-3-oxo-7-(trifluoromethyl)isoindolin-5-yl)methyl)-3-isopropylpiperazine-1-carboxylate in DCM (3 mL) was added trifluoroacetic acid (1 mL). The reaction was stirred at rt for 1 h then was diluted with toluene (2 mL) and concentrated. To the crude in methanol (5 mL) was added sodium acetate (1.779 g). To the stirring mixture was added formaldehyde (37% in water) (347.4 μL) and the reaction was stirred for 10 minutes at rt then was added sodium triacetoxyborohydride (449.8 mg) and stirred for another 20 minutes. The mixture was concentrated with DMSO (2 mL) and the crude mixture was purified by chromatography on C18 silica gel (0-60% gradient of acetonitrile in ammonium formate buffer, pH=3.8). Appropriate fractions were concentrated, frozen and lyophilized to provide Compound 236, (S)-2-(4-(3,3-difluoro-1-((4-methyl-4H-1,2,4-triazol-3-yl)methyl)cyclobutyl)-6-(ethylamino)pyridin-2-yl)-6-((2-isopropyl-4-methylpiperazin-1-yl)methyl)-4-(trifluoromethyl)isoindolin-1-one formate (16.5 mg). LCMS (ESI) m/z: 661.5 [M+H]⁺¹H NMR (400 MHz, DMSO-d6) δ 8.19 (d, J=8.5 Hz, 1H), 8.17 (s, 1H), 7.95 (s, 1H), 7.89 (s, 1H), 7.42 (d, J=0.9 Hz, 1H), 0.6.98 (d, J=4.0 Hz, 1H), 6.58 (t, J=5.5 Hz, 1H), 5.80 (s, 1H), 5.12 (s, 2H), 4.18 (d, J=14.3 Hz, 1H), 3.33 (dd, J=28.6, 23.8 Hz, 7H), 3.19-3.07 (m, 3H), 3.01 (s, 3H), 2.87 (q, J=13.8 Hz, 2H), 2.59 (t, J=10.5 Hz, 2H), 2.26-2.17 (m, 3H), 2.13 (s, 3H), 1.96-1.88 (m, 2H), 1.09 (t, J=7.1 Hz, 3H), 0.91 (d, J=6.6 Hz, 3H), 0.86 (d, J=6.6 Hz, 3H).

### Example 49: Compound 237

Compound 237 (2-(3-(3-(fluoro(4-methyl-1H-pyrazol-5-yl)methyl)oxetan-3-yl)-phenyl)-6-(((S)-2-isopropyl-4-methylpiperazin-1-yl)methyl)-4-(trifluoromethyl)isoindolin-1-one) can be synthesized according to Scheme 47, FIG. 29.

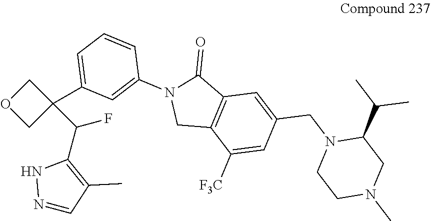 [SMILES: Cc1cn[nH]c1C(F)C1(c2cccc(N3Cc4c(cc(CN5CCN(C)C[C@@H]5C(C)C)cc4C(F)(F)F)C3=O)c2)COC1]

A first intermediate, 4-methyl-1-((2-(trimethylsilyl)ethoxy)methyl)-1H-pyrazole, can be made as follows. To a stirred solution of 4-methylpyrazole (200 mg, 2.44 mmol) in THF (8.1 mL) at 0° C. was added sodium hydride (60% in mineral oil) (102.3 mg, 2.56 mmol). After stirring for 30 minutes at 0° C., 2-(trimethylsilyl)ethoxymethyl chloride (0.43 mL, 2.44 mmol) was added. The reaction was stirred for 16 h at rt. The reaction mixture was treated with water (15 mL) and EtOAc (20 mL). The phases were separated and the aqueous layer was extracted with EtOAc (25 mL×2). The combined organic layers were dried over sodium sulfate, filtered and concentrated. The crude product was purified by chromatography on silica gel (0-100% of EtOAc in heptanes) to afford 4-methyl-1-((2-(trimethylsilyl)-ethoxy)methyl)-1H-pyrazole (320 mg, 62% yield) as a clear colourless oil. ¹H NMR (400 MHz, CDCl₃) δ 7.35 (s, 1H), 7.33 (s, 1H), 5.36 (s, 2H), 3.58-3.43 (m, 2H), 2.09 (s, 3H), 0.95-0.81 (m, 2H), −0.03 (s, 9H).

A second intermediate, (3-(3-bromophenyl)oxetan-3-yl)(4-methyl-1-((2-(trimethylsilyl)ethoxy)methyl)-1H-pyrazol-5-yl)methanol, can be made as follows. A solution of 2.5 M n-BuLi in hexane (0.43 mL, 1.08 mmol) was added dropwise to a solution of 4-methyl-1-((2-(trimethylsilyl)ethoxy)methyl)-1H-pyrazole (229.0 mg, 1.08 mmol) in anhydrous DME (10.7 mL) at −50° C. The resulting mixture was stirred at −50° C. for 1 h before a solution of 3-(3-bromophenyl)oxetane-3-carbaldehyde (130 mg, 0.54 mmol) in DME (3 mL) was added dropwise. The reaction was gradually allowed to warm to 0° C. over 1 h. The reaction was quenched with water and diluted with CHCl₃/IPA 3:1 mixture. The layers were separated, the aqueous phase was extracted with CHCl₃/IPA 3:1 mixture (3×). The combined organic phases were dried over magnesium sulfate, filtered and concentrated. The crude product was purified by chromatography on silica gel (0-100% of EtOAc in heptanes) to afford (3-(3-bromophenyl)oxetan-3-yl)(4-methyl-1-((2-(trimethylsilyl)ethoxy)-methyl)-1H-pyrazol-5-yl)methanol (73 mg, 30% yield) as a clear colourless oil. LCMS (ESI) m/z: 452.9 [M+H]⁺.

A third intermediate, 5-((3-(3-bromophenyl)oxetan-3-yl)fluoromethyl)-4-methyl-1-((2-(trimethylsilyl)ethoxy)methyl)-1H-pyrazole, can be made as follows. To a solution of (3-(3-bromophenyl)oxetan-3-yl)(4-methyl-1-((2-(trimethylsilyl)ethoxy)methyl)-1H-pyrazol-5-yl)methanol (73 mg, 0.16 mmol) in DCM (2.3 mL) was added deoxofluor (50% w/w in toluene) (0.24 mL, 0.53 mmol) dropwise at 0° C. (internal monitoring kept temperature below 5° C.). After 2 h, the reaction was cooled back to 0° C. and was quenched with slow addition of water (5 mL). The product was extracted with 30% IPA in CHCl₃ (3×5 mL) and the combined organic layers were dried over sodium sulfate, filtered and concentrated. The crude product was purified by chromatography on silica gel (0-100% of EtOAc in heptanes) to afford 5-((3-(3-bromophenyl)oxetan-3-yl)fluoromethyl)-4-methyl-1-((2-(trimethylsilyl)-ethoxy)methyl)-1H-pyrazole (45 mg, 61% yield) as a clear colourless oil. LCMS (ESI) m/z: 454.9 [M+H]⁺.

A fourth intermediate, 2-(3-(3-(fluoro(4-methyl-1-((2-(trimethylsilyl)ethoxy)methyl)-1H-pyrazol-5-yl)methyl)oxetan-3-yl)phenyl)-6-(((S)-2-isopropyl-4-methylpiperazin-1-yl)methyl)-4-(trifluoromethyl)isoindolin-1-one, can be made as follows. A vial was charged with (S)-6-((2-isopropyl-4-methylpiperazin-1-yl)methyl)-4-(trifluoromethyl)isoindolin-1-one (Intermediate S; 36.9 mg, 0.10 mmol), 5-((3-(3-bromophenyl)oxetan-3-yl)fluoromethyl)-4-methyl-1-((2-(trimethylsilyl)ethoxy)methyl)-1H-pyrazole (45 mg, 0.10 mmol), Me₄tButylXphos (9.5 mg, 0.02 mmol), Tris(dibenzylideneacetone)dipalladium(0) (9.1 mg, 0.01 mmol) and cesium carbonate (64.8 mg, 0.20 mmol). The vial was purged with nitrogen before degassed toluene (0.99 mL) was added and the vial was sealed. The reaction mixture was stirred at 110° C. for 16 h and cooled down to rt. 3:1 CHCl₃/IPA was added and the reaction was filtered through a 0.45 μm PTFE filter. Water (10 mL) was added to the residue and the product was extracted with 3:1 CHCl₃/IPA (3×10 mL). The organic layers were combined, dried over sodium sulfate, filtered, and concentrated. The crude product was purified by chromatography on silica gel (0-10% of MeOH in DCM) to afford 2-(3-(3-(fluoro(4-methyl-1-((2-(trimethylsilyl)ethoxy)methyl)-1H-pyrazol-5-yl)methyl)oxetan-3-yl)phenyl)-6-(((S)-2-isopropyl-4-methylpiperazin-1-yl)methyl)-4-(trifluoromethyl)isoindolin-1-one (17 mg, 24% yield) as yellow solid. LCMS (ESI) m/z: 730.3 [M+H]⁺

To 2-(3-(3-(fluoro(4-methyl-1-((2-(trimethylsilyl)ethoxy)methyl)-1H-pyrazol-5-yl)methyl) oxetan-3-yl)phenyl)-6-(((S)-2-isopropyl-4-methylpiperazin-1-yl)methyl)-4-(trifluoro-methyl) isoindolin-1-one (17 mg, 0.02 mmol) was added triethylsilane (50 μL, 0.31 mmol) followed by trifluoroacetic acid (0.10 mL, 1.3 mmol). The reaction was stirred at rt for 3 h. Excess TFA was removed and the reaction was diluted with DCM. 1 M NaOH solution was added to reach pH 12. The product was extracted with 30% IPA in CHCl₃ (3×). The organics were combined, dried over sodium sulfate, filtered and concentrated. The crude product was purified by semi-prep. LCMS (25-45% gradient of acetonitrile in ammonium formate buffer, pH=3.8). Appropriate fractions were concentrated, frozen and lyophilized to provide Compound 237, 2-(3-(3-(fluoro(4-methyl-1H-pyrazol-5-yl)methyl)oxetan-3-yl)phenyl)-6-(((S)-2-isopropyl-4-methylpiperazin-1-yl)methyl)-4-(trifluoromethyl)isoindolin-1-one (3.8 mg, 27% yield). LCMS (ESI) m/z: 600.4 [M+H]⁺¹H NMR (400 MHz, DMSO-d6) δ 10.58-10.51 (m, 1H), 7.96 (s, 1H), 7.89 (s, 1H), 7.80 (d, J=8.6 Hz, 1H), 7.49 (s, 1H), 7.35-7.19 (m, 2H), 6.86 (d, J=7.4 Hz, 1H), 5.89 (d, J=46.2 Hz, 1H), 5.33 (d, J=6.4 Hz, 1H), 5.16 (d, J=5.8 Hz, 1H), 5.09-4.93 (m, 3H), 4.77-4.69 (m, 1H), 4.19 (d, J=14.4 Hz, 1H), 3.35 (d, J=14.3 Hz, 2H), 2.68-2.53 (m, 2H), 2.34-2.14 (m, 4H), 2.12 (s, 3H), 2.00-1.73 (m, 2H), 1.47 (ap s, 2H), 0.90 (d, J=6.7 Hz, 3H), 0.86 (d, J=6.6 Hz, 3H).

### Example 50: Compound 238

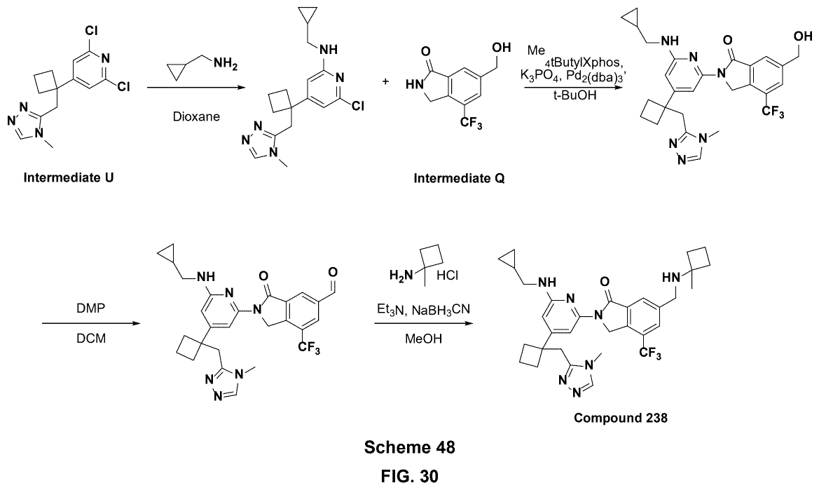

Compound 238 (2-(6-((cyclopropylmethyl)amino)-4-(1-((4-methyl-4H-1,2,4-triazol-3-yl)methyl)cyclobutyl)pyridin-2-yl)-6-(((1-methylcyclobutyl)amino)methyl)-4-(trifluoromethyl)-isoindolin-1-one) can be synthesized according to Scheme 48, FIG. 30.

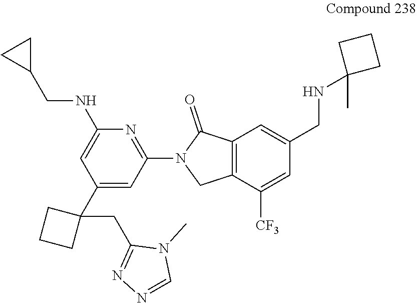 [SMILES: Cn1cnnc1CC1(c2cc(NCC3CC3)nc(N3Cc4c(cc(CNC5(C)CCC5)cc4C(F)(F)F)C3=O)c2)CCC1]

A first intermediate, (6-chloro-N-(cyclopropylmethyl)-4-(1-((4-methyl-4H-1,2,4-triazol-3-yl)methyl)-cyclobutyl)pyridin-2-amine) can be synthesized as follows. In a sealed tube and to a stirred solution of 2,6-dichloro-4-(1-((4-methyl-4H-1,2,4-triazol-3-yl)methyl)-cyclobutyl)pyridine (Intermediate U; 120 mg, 0.40 mmol) in 1,4-dioxane (0.41 mL) was added cyclopropanemethylamine (0.7 mL, 8.08 mmol). The resulting reaction mixture was stirred at 80° C. for 72 h. The reaction was cooled down to rt and concentrated. The crude residue was dissolved in DCM, washed with water and brine, dried over sodium sulfate, filtered and concentrated to obtain 6-chloro-N-(cyclopropylmethyl)-4-(1-((4-methyl-4H-1,2,4-triazol-3-yl)methyl)cyclobutyl) pyridin-2-amine (125 mg, 93% yield) which was used in the next step without further purification. LCMS (ESI) m/z: 332.2, 334.1 [M+H]⁺.

A second intermediate, 2-(6-((cyclopropylmethyl)amino)-4-(1-((4-methyl-4H-1,2,4-triazol-3-yl)methyl)cyclobutyl)pyridin-2-yl)-6-(hydroxymethyl)-4-(trifluoromethyl)isoindolin-1-one, can be prepared as follows. A microwave vial was charged with 6-chloro-N-(cyclo-propylmethyl)-4-(1-((4-methyl-4H-1,2,4-triazol-3-yl)methyl)cyclobutyl)pyridin-2-amine (125 mg, 0.38 mmol), 6-(hydroxymethyl)-4-(trifluoromethyl)isoindolin-1-one (Intermediate Q; 95.8 mg, 0.41 mmol), Me₄tButylXphos (18.1 mg, 0.04 mmol), Pd₂(dba)₃ (17.3 mg, 0.02 mmol) and K₃PO₄ (239.9 mg, 1.13 mmol). The vial was purged with nitrogen before degassed anhydrous tert-butanol (2 mL) was added and the vial was sealed. The reaction mixture was stirred at 110° C. for 15 h. The reaction was cooled down to rt and concentrated. The crude product was purified by chromatography on silica gel (0-30% of MeOH in DCM) to afford 2-(6-((cyclopropylmethyl)amino)-4-(1-((4-methyl-4H-1,2,4-triazol-3-yl)methyl)cyclobutyl)pyridin-2-yl)-6-(hydroxymethyl)-4-(trifluoromethyl)isoindolin-1-one (139 mg, 70% yield). LCMS (ESI) m/z: 527.1 [M+H]⁺.

A third intermediate, 2-(6-((cyclopropylmethyl)amino)-4-(1-((4-methyl-4H-1,2,4-triazol-3-yl)methyl)cyclobutyl)pyridin-2-yl)-3-oxo-7-(trifluoromethyl)isoindoline-5-carbaldehyde, can be prepared as follows. Dess-Martin periodinane (“DMP”; 167.9 mg, 0.40 mmol) was added to a suspension of 2-(6-((cyclopropylmethyl)amino)-4-(1-((4-methyl-4H-1,2,4-triazol-3-yl)methyl)cyclobutyl)pyridin-2-yl)-6-(hydroxymethyl)-4-(trifluoromethyl)-isoindolin-1-one (139 mg, 0.26 mmol) in DCM (1.5 mL) at 0° C. The resulting mixture was allowed to warm to rt and stirred for 3 h. The reaction was quenched with saturated aqueous Na₂S₂O₃ solution and saturated aqueous NaHCO₃ solution and the resulting mixture was stirred vigorously for 30 minutes. The layers were separated and the aqueous phase was extracted with 4:1 CHCl₃/IPA (3×). The combined organic phases were dried over magnesium sulfate, filtered and concentrated. The crude product was purified by chromatography on silica gel (1-10% of MeOH in DCM) to afford 2-(6-((cyclopropylmethyl)-amino)-4-(1-((4-methyl-4H-1,2,4-triazol-3-yl)methyl)cyclobutyl)pyridin-2-yl)-3-oxo-7-(trifluoromethyl)isoindoline-5-carbaldehyde (70 mg, 51% yield). LCMS (ESI) m/z: 525.0 [M+H]⁺

To form Compound 238, in a microwave vial 2-(6-((cyclopropylmethyl)amino)-4-(1-((4-methyl-4H-1,2,4-triazol-3-yl)methyl)cyclobutyl)pyridin-2-yl)-3-oxo-7-(trifluoromethyl)isoindoline-5-carbaldehyde (52.5 mg, 0.10 mmol), triethylamine (83.6 μL, 0.60 mmol) and 1-methylcyclobutanamine hydrochloride (73 mg, 0.60 mmol) were dissolved in methanol (1 mL). The resulting reaction mixture was heated in a microwave oven for 5 minutes at 100° C. At rt, sodium cyanoborohydride (18.9 mg, 0.30 mmol) was added. The reaction was once again heated in a microwave oven at 100° C. for 1 h. The reaction was concentrated and the crude residue was purified by chromatography on C18 silica gel (0-100% acetonitrile in ammonium formate buffer, pH=3.8). Appropriate fractions were concentrated, frozen and lyophilized to afford Compound 238, 2-(6-((cyclopropylmethyl)-amino)-4-(1-((4-methyl-4H-1,2,4-triazol-3-yl)methyl)cyclobutyl)pyridin-2-yl)-6-(((1-methyl-cyclobutyl)amino)methyl)-4-(trifluoromethyl)isoindolin-1-one (14 mg, 24% yield). LCMS (ESI) m/z: 594.1 [M+H]⁺¹H NMR (400 MHz, DMSO-d6) δ 8.17 (s, 1H), 8.00 (d, J=9.8 Hz, 2H), 7.37 (s, 1H), 6.60 (t, J=5.6 Hz, 1H), 5.78 (s, 1H), 5.12 (s, 2H), 3.82 (s, 2H), 3.14 (s, 2H), 3.05 (t, J=6.2 Hz, 2H), 3.02 (s, 3H), 2.43 (m, 2H), 2.25 (m, 2H), 2.13-1.90 (m, 3H), 1.85-1.61 (m, 5H), 1.23 (s, 3H), 1.08-0.92 (m, 1H), 0.47-0.32 (m, 2H), 0.24-0.12 (m, 2H).

### Example 51: Compound 239

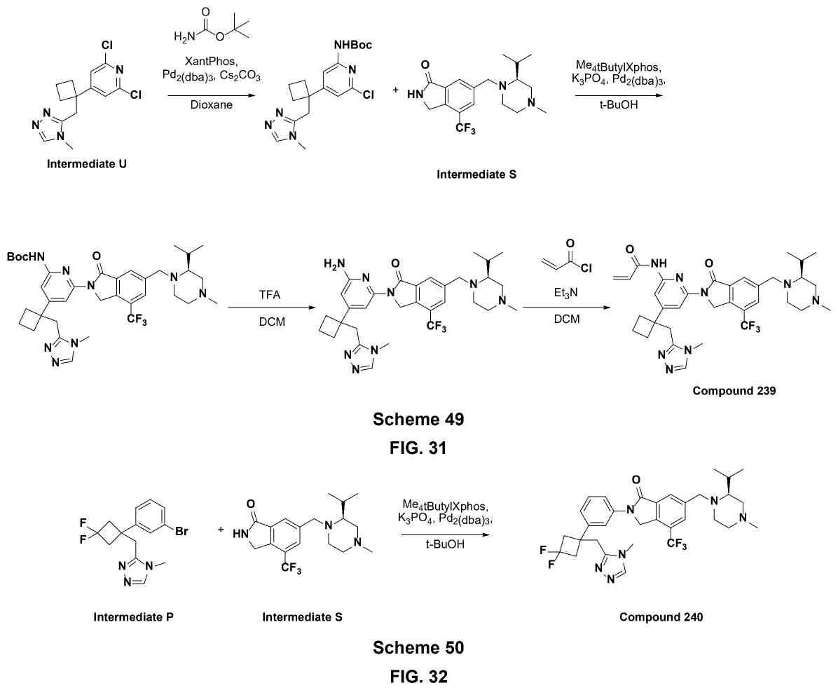

Compound 239 ((S)—N-(6-(6-((2-isopropyl-4-methylpiperazin-1-yl)methyl)-1-oxo-4-(trifluoromethyl)isoindolin-2-yl)-4-(1-((4-methyl-4H-1,2,4-triazol-3-yl)methyl)cyclobutyl)-pyridin-2-yl)acrylamide) can be synthesized according to Scheme 49, FIG. 31.

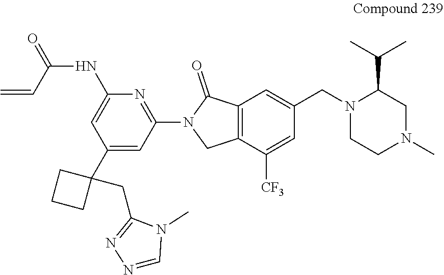 [SMILES: C=CC(=O)Nc1cc(C2(Cc3nncn3C)CCC2)cc(N2Cc3c(cc(CN4CCN(C)C[C@@H]4C(C)C)cc3C(F)(F)F)C2=O)n1]

A first intermediate, tert-butyl (6-chloro-4-(1-((4-methyl-4H-1,2,4-triazol-3-yl)methyl)cyclobutyl)pyridin-2-yl)carbamate, can be formed as follows. A microwave vial was charged with 2,6-dichloro-4-(1-((4-methyl-4H-1,2,4-triazol-3-yl)methyl)cyclobutyl)-pyridine (Intermediate U; 300 mg, 1.01 mmol), Pd₂(dba)₃.CHCl₃ (52.3 mg, 0.05 mmol), cesium carbonate (661.9 mg, 2.02 mmol), Xantphos (58.4 mg, 0.10 mmol) and tert-butyl carbamate (130.1 mg, 1.11 mmol). The vial was purged with nitrogen before degassed 1,4-dioxane (10.1 mL) was added and the vial was sealed. The reaction mixture was stirred at 90° C. for 15 h. The vial was cooled down to rt and concentrated. The crude product was purified by chromatography on silica gel (0-10% of MeOH in DCM) to afford tert-butyl (6-chloro-4-(1-((4-methyl-4H-1,2,4-triazol-3-yl)methyl)cyclobutyl)pyridin-2-yl)carbamate (348 mg, 91% yield) as a white solid. LCMS (ESI) m/z: 378.0, 379.5 [M+H]⁺

A second intermediate, (S)-tert-butyl (6-(6-((2-isopropyl-4-methylpiperazin-1-yl)methyl)-1-oxo-4-(trifluoromethyl)isoindolin-2-yl)-4-(1-((4-methyl-4H-1,2,4-triazol-3-yl)methyl)cyclobutyl)pyridin-2-yl)carbamate, can be made as follows. A microwave vial was charged with tert-butyl (6-chloro-4-(1-((4-methyl-4H-1,2,4-triazol-3-yl)methyl)cyclobutyl)-pyridin-2-yl)carbamate (38 mg, 0.10 mmol), (S)-6-((2-isopropyl-4-methylpiperazin-1-yl)methyl)-4-(trifluoromethyl)isoindolin-1-one (Intermediate S; 46.3 mg, 0.11 mmol), Me₄^{t}ButylXphos (4.8 mg, 0.01 mmol), Pd₂(dba)₃ (4.6 mg, 0.01 mmol) and K₃PO₄ (64.0 mg, 0.30 mmol). The vial was purged with nitrogen before degassed anhydrous tert-butanol (1.0 mL) was added and the vial was sealed. The reaction mixture was stirred at 110° C. for 20 h. The vial was cooled down to rt and concentrated. The crude product was purified by chromatography on silica gel (0-30% of MeOH in DCM) to afford (S)-tert-butyl (6-(6-((2-isopropyl-4-methylpiperazin-1-yl)methyl)-1-oxo-4-(trifluoromethyl)isoindolin-2-yl)-4-(1-((4-methyl-4H-1,2,4-triazol-3-yl)methyl)cyclobutyl)pyridin-2-yl)carbamate (50 mg, 71% yield) as a white solid. LCMS (ESI) m/z: 697.3 [M+H]⁺.

A third intermediate, (S)-2-(6-amino-4-(1-((4-methyl-4H-1,2,4-triazol-3-yl)methyl)cyclobutyl)pyridin-2-yl)-6-((2-isopropyl-4-methylpiperazin-1-yl)methyl)-4-(trifluoromethyl)isoindolin-1-one, can be made as follows. To a stirred solution (S)-tert-butyl (6-(6-((2-isopropyl-4-methylpiperazin-1-yl)methyl)-1-oxo-4-(trifluoromethyl)isoindolin-2-yl)-4-(1-((4-methyl-4H-1,2,4-triazol-3-yl)methyl)cyclobutyl)pyridin-2-yl)carbamate (50.mg, 0.07 mmol) in DCM (0.72 mL) was added trifluoroacetic acid (0.27 mL, 3.59 mmol). The resulting reaction mixture was stirred at rt for 1 h. The reaction was concentrated and the crude residue was purified by chromatography on C18 silica gel (0-100% acetonitrile in ammonium formate buffer, pH=3.8). Appropriate fractions were concentrated, frozen and lyophilized to afford (S)-2-(6-amino-4-(1-((4-methyl-4H-1,2,4-triazol-3-yl)methyl)cyclobutyl)pyridin-2-yl)-6-((2-isopropyl-4-methylpiperazin-1-yl)methyl)-4-(trifluoromethyl)isoindolin-1-one (30 mg, 70% yield) as a white solid. LCMS (ESI) m/z: 597.1 [M+H]⁺

To make Compound 239, to a solution of (S)-2-(6-amino-4-(1-((4-methyl-4H-1,2,4-triazol-3-yl)methyl)cyclobutyl)pyridin-2-yl)-6-((2-isopropyl-4-methylpiperazin-1-yl)methyl)-4-(trifluoromethyl)isoindolin-1-one (30 mg, 0.05 mmol), triethylamine (140 μL, 0.10 mmol) in DCM (1 mL) at −78° C. was added acryloyl chloride (4.5 μL, 0.06 mmol) under nitrogen. The reaction mixture was slowly warm to 0° C. over 3 h. The reaction was concentrated and the crude residue was purified by chromatography on C18 silica gel (0-100% acetonitrile in ammonium formate buffer, pH=3.8). Appropriate fractions were concentrated, frozen and lyophilized to afford (S)—N-(6-(6-((2-isopropyl-4-methylpiperazin-1-yl)methyl)-1-oxo-4-(trifluoromethyl)isoindolin-2-yl)-4-(1-((4-methyl-4H-1,2,4-triazol-3-yl)methyl)cyclobutyl)-pyridin-2-yl)acrylamide (3.5 mg, 11% yield). LCMS (ESI) m/z: 651.1 [M+H]⁺¹H NMR (400 MHz, DMSO-d6) δ 10.53 (s, 1H), 8.15 (s, 1H), 7.98 (s, 1H), 7.91 (d, J=7.9 Hz, 2H), 7.72 (d, J=8.6 Hz, 1H), 6.65 (dd, J=17.0, 10.2 Hz, 1H), 6.28 (dd, J=17.0, 1.8 Hz, 1H), 5.77 (dd, J=10.2, 1.8 Hz, 1H), 5.18 (s, 2H), 4.19 (d, J=14.4 Hz, 1H), 3.36 (d, J=14.4 Hz, 2H), 3.22 (s, 2H), 3.03 (s, 3H), 2.58 (dd, J=24.8, 14.7 Hz, 2H), 2.37-2.16 (m, 6H), 2.15-2.03 (m, 5H), 1.88 (ddd, J=19.9, 14.8, 7.6 Hz, 3H), 0.91 (d, J=6.6 Hz, 3H), 0.87 (d, J=6.6 Hz, 3H.

### Example 52: Compound 240

Compound 240 ((S)-2-(3-(3,3-difluoro-1-((4-methyl-4H-1,2,4-triazol-3-yl)methyl)-cyclobutyl)phenyl)-6-((2-isopropyl-4-methylpiperazin-1-yl)methyl)-4-(trifluoromethyl)iso-indolin-1-one) can be synthesized according to Scheme 50, FIG. 32.

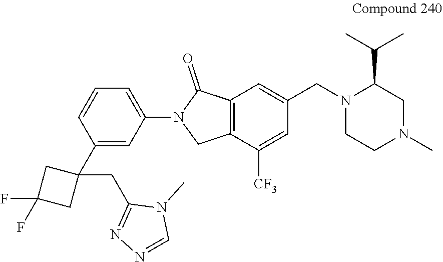 [SMILES: CC(C)[C@H]1CN(C)CCN1Cc1cc2c(c(C(F)(F)F)c1)CN(c1cccc(C3(Cc4nncn4C)CC(F)(F)C3)c1)C2=O]

A microwave vial was charged with 3-((1-(3-bromophenyl)-3,3-difluorocyclobutyl)-methyl)-4-methyl-4H-1,2,4-triazole (Intermediate P; 52.1 mg, 0.15 mmol), (S)-6-((2-isopropyl-4-methylpiperazin-1-yl)methyl)-4-(trifluoromethyl)isoindolin-1-one (Intermediate S; 50 mg, 0.15 mmol), Me₄tButylXphos (7.3 mg, 0.02 mmol), Pd₂(dba)₃ (7.0 mg, 0.01 mmol) and K₃PO₄ (96.9 mg, 0.46 mmol). The vial was purged with nitrogen before degassed anhydrous tert-butanol (0.76 mL) was added and the vial was sealed. The reaction mixture was stirred at 110° C. for 15 h. The reaction was concentrated and the crude residue was purified by chromatography on C18 silica gel (0-100% acetonitrile in 0.5% formic acid buffer). Appropriate fractions were concentrated, frozen and lyophilized to afford (S)-2-(3-(3,3-difluoro-1-((4-methyl-4H-1,2,4-triazol-3-yl)methyl) cyclobutyl)phenyl)-6-((2-isopropyl-4-methylpiperazin-1-yl)methyl)-4-(trifluoromethyl) isoindolin-1-one (22 mg, 25% yield). LCMS (ESI) m/z: 590.2 [M+H]⁺. ¹H NMR (400 MHz, DMSO-d6) δ 8.16 (s, 1H), 7.93 (dd, J=8.2, 1.9 Hz, 1H), 7.45 (s, 1H), 7.41 (m, 1H), 7.34 (t, J=8.0 Hz, 1H), 6.79 (d, J=7.7 Hz, 1H), 5.14 (s, 2H), 4.00 (d, J=14.8 Hz, 1H), 3.51 (d, J=14.7 Hz, 1H), 3.31-3.19 (m, 4H), 3.08-2.89 (m, 2H), 2.71 (s, 3H), 2.67 (m, 1H), 2.61-2.50 (m, 2H), 2.36 (m, 1H), 2.30-2.20 (m, 2H), 2.20-2.14 (m, 1H), 2.12 (s, 3H), 1.94 (m, 1H), 1.82 (t, J=10.3 Hz, 1H), 1.13-1.00 (m, 4H), 0.89 (d, J=6.8 Hz, 3H), 0.84 (d, J=6.8 Hz, 3H).

### Example 53: Compound 241

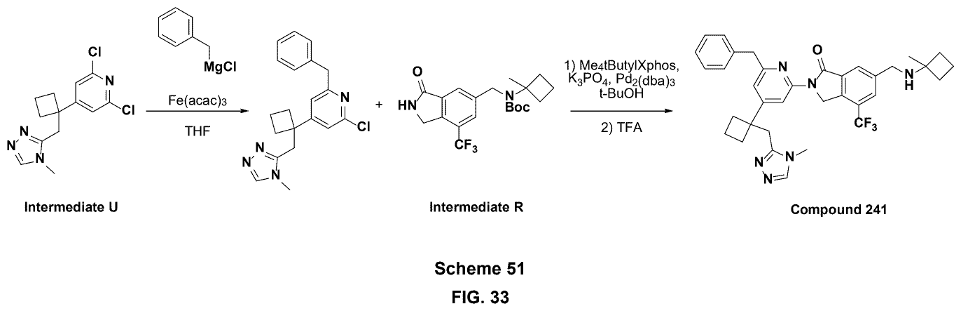

Compound 241 (2-(6-benzyl-4-(1-((4-methyl-4H-1,2,4-triazol-3-yl)methyl)cyclo-butyl)pyridin-2-yl)-6-(((1-methylcyclobutyl)amino)methyl)-4-(trifluoromethyl)isoindolin-1-one) can be synthesized according to Scheme 51, FIG. 33.

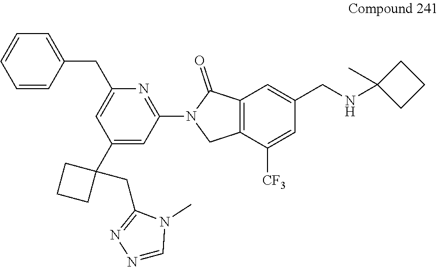 [SMILES: Cn1cnnc1CC1(c2cc(Cc3ccccc3)nc(N3Cc4c(cc(CNC5(C)CCC5)cc4C(F)(F)F)C3=O)c2)CCC1]

A first intermediate, 2-benzyl-6-chloro-4-(1-((4-methyl-4H-1,2,4-triazol-3-yl)methyl)cyclobutyl)pyridine, can be made as follows. To a vial was added 2,6-dichloro-4-(1-((4-methyl-4H-1,2,4-triazol-3-yl)methyl)cyclobutyl) pyridine (Intermediate U; 100 mg, 0.34 mmol) and Fe(acac)₃ (5.9 mg, 0.02 mmol). The vial was sealed with a septum cap, evacuated, purged with nitrogen and the mixture dissolved in THF (5.0 mL). The reaction mixture was treated dropwise with 2 M benzylmagnesium chloride solution in THF (202 μL, 0.40 mmol) at 0° C. After stirring for 2 h, the reaction was quenched by adding water (5 mL) and extracted with EtOAc (3×15 mL). The organic layers were combined, dried over sodium sulfate, filtered and concentrated to afford 2-benzyl-6-chloro-4-(1-((4-methyl-4H-1,2,4-triazol-3-yl)methyl)cyclobutyl)pyridine (105 mg, 88% yield). Used in the next step without further purification. LCMS (ESI) m/z: 353.2 [M+H]⁺.

To make Compound 241, a microwave vial equipped with a stir bar was charged with K₃PO₄ (96.9 mg, 0.46 mmol) and flame dried under nitrogen. 2-benzyl-6-chloro-4-(1-((4-methyl-4H-1,2,4-triazol-3-yl)methyl)cyclobutyl)pyridine (53.7 mg, 0.11 mmol), tert-butyl (1-methylcyclobutyl)((3-oxo-7-(trifluoromethyl)isoindolin-5-yl)methyl) carbamate (Intermediate R; 50 mg, 0.13 mmol), tris(dibenzylideneacetone)dipalladium(0) (5.2 mg, 0.01 mmol) and Me₄tButylXphos (5.5 mg, 0.01 mmol) were then added and the vial was flushed with nitrogen for several minutes. Degassed tert-butanol (1 mL) was added via syringe, the vial was capped and the reaction mixture was stirred at 110° C. for 16 h, then cooled down to rt. Reaction was diluted with water and EtOAc and mixture was passed through a pad of celite. Combined organic layers were dried over sodium sulfate, filtered and evaporated. The crude was dissolved in DCM (2 mL), trifluoroacetic acid (0.5 mL) was added and stirred for 1 h. Solvent was evaporated and the crude mixture was dissolved in DCM (2 mL), trimethylamine (300 μL) was added and stirred for 15 minutes. Solvent was evaporated and the crude residue was purified by chromatography on C18 silica gel (0-100% acetonitrile in 0.5% formic acid buffer). Appropriate fractions were concentrated, frozen and lyophilized to afford 2-(6-benzyl-4-(1-((4-methyl-4H-1,2,4-triazol-3-yl)methyl)cyclobutyl)pyridin-2-yl)-6-(((1-methylcyclobutyl)amino)methyl)-4-(trifluoromethyl)isoindolin-1-one (29 mg, 38% yield). LCMS (ESI) m/z: 615.2 [M+H]⁺. ¹H NMR (400 MHz, DMSO-d6) δ 8.14-8.10 (m, 2H), 8.05 (s, 1H), 8.02 (s, 1H), 7.35-7.17 (m, 5H), 6.66 (s, 1H), 5.19 (s, 2H), 4.00 (s, 4H), 3.20 (s, 2H), 2.80 (s, 3H), 2.54-2.52 (m, 1H), 2.34-2.10 (m, 5H), 1.84-1.73 (m, 5H), 1.38-1.34 (m, 3H).

### Example 54: Compound 242

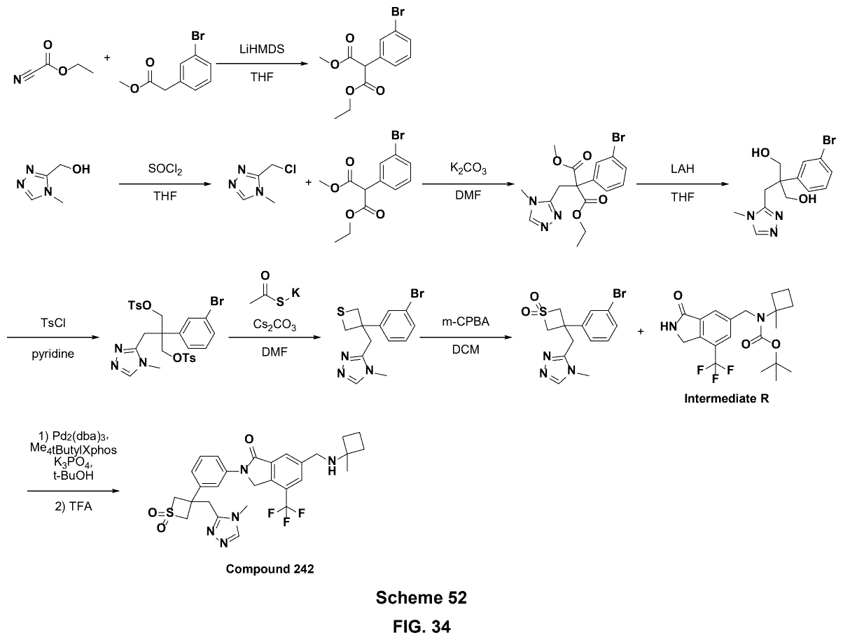

Compound 242 (2-(3-(3-((4-methyl-4H-1,2,4-triazol-3-yl)methyl)-1,1-dioxido-thietan-3-yl)phenyl)-6-(((1-methylcyclobutyl)amino)methyl)-4-(trifluoromethyl)isoindolin-1-one) can be synthesized according to Scheme 52, FIG. 34.

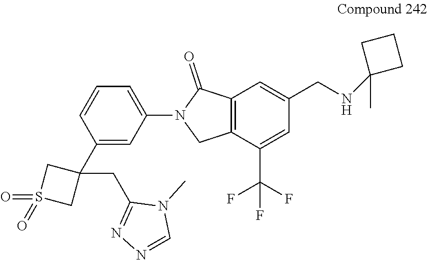 [SMILES: Cn1cnnc1CC1(c2cccc(N3Cc4c(cc(CNC5(C)CCC5)cc4C(F)(F)F)C3=O)c2)CS(=O)(=O)C1]

A first intermediate, 1-ethyl 3-methyl 2-(3-bromophenyl)malonate, can be made as follows. To a solution of methyl 2-(3-bromophenyl)acetate (3 g, 13.1 mmol) in anhydrous THF (20 mL) at 0° C., was added slowly 1 M lithium bis(trimethylsilyl)amide solution in THF (32.7 mL, 32.74 mmol) under nitrogen atmosphere. The reaction was stirred at rt for 1 h. Then, ethyl cyanoformate (3.9 mL, 39.29 mmol) was added dropwise at 0° C. The mixture was allowed to warm to rt and stirred for 2 h. Reaction was acidified to pH 5 with 1 N HCl solution. EtOAc was added, organic phase was separated, washed with water and brine, dried over sodium sulfate, filtered and concentrated. The residue was purified by chromatography on silica gel (0-100% EtOAc in heptanes) to get 1-ethyl 3-methyl 2-(3-bromophenyl)malonate (3.65 g, 93% yield). LCMS (ESI) m/z: 303.0 [M+H]⁺

A second intermediate, 3-(chloromethyl)-4-methyl-4H-1,2,4-triazole, can be made as follows. (4-methyl-4H-1,2,4-triazol-3-yl)methanol (350 mg, 3.09 mmol) was dissolved in thionyl chloride (1.8 mL, 24.75 mmol) and stirred for 2 h at 50° C. Thionyl chloride was evaporated to obtain 3-(chloromethyl)-4-methyl-4H-1,2,4-triazole (410 mg, 100% yield) used in the next step without further purification. LCMS (ESI) m/z: 132.3 [M+H]⁺

A third intermediate, 1-ethyl 3-methyl 2-(3-bromophenyl)-2-((4-methyl-4H-1,2,4-triazol-3-yl)methyl)malonate, can be made as follows. To a DMF (20 mL) solution of 1-ethyl 3-methyl 2-(3-bromophenyl)malonate (801.1 mg, 2.66 mmol) was added K₂CO₃ (1.838 g, 13.3 mmol) and mixture was stirred for 20 min. 3-(chloromethyl)-4-methyl-4H-1,2,4-triazole (350 mg, 2.66 mmol) dissolved in DMF (2 mL) was added to the reaction mixture and was stirred for 16 h at 40° C. Reaction was quenched with water and diluted with EtOAc. Organic layer was separated, washed with water and brine, dried over sodium sulfate, filtered and concentrated. The crude was dissolved in DCM and crystallized with Ether and heptane to afford 1-ethyl 3-methyl 2-(3-bromophenyl)-2-((4-methyl-4H-1,2,4-triazol-3-yl)methyl)malonate (855 mg, 81% yield) LCMS (ESI) m/z: 397.9 [M+H]⁺

A fourth intermediate, 2-(3-bromophenyl)-2-((4-methyl-4H-1,2,4-triazol-3-yl)methyl)propane-1,3-diol, can be made as follows. To a THF (10 mL) solution of 1-ethyl 3-methyl 2-(3-bromophenyl)-2-((4-methyl-4H-1,2,4-triazol-3-yl)methyl)malonate (850 mg, 2.15 mmol) was added lithium aluminum hydride (211.8 mg, 6.44 mmol) at 0° C. The reaction was stirred for 1 h at 0° C., warmed to rt and stirred for an additional 2 h. The reaction was poured into cold saturated aqueous NH₄Cl solution and diluted with EtOAc. The desired product was water soluble, but was extracted with EtOAc/MeOH (10%). Combined organic layers were washed with brine, dried over sodium sulfate, filtered and concentrated. The crude residue was purified by chromatography on C18 silica gel (acetonitrile in ammonium bicarbonate buffer, pH=10). Appropriate fractions were concentrated, frozen and lyophilized to afford 2-(3-bromophenyl)-2-((4-methyl-4H-1,2,4-triazol-3-yl)methyl)propane-1,3-diol (170 mg, 24% yield). LCMS (ESI) m/z: 328.1 [M+H]⁺

A fifth intermediate, 2-(3-bromophenyl)-2-((4-methyl-4H-1,2,4-triazol-3-yl)-methyl)propane-1,3-diyl bis(4-methylbenzenesulfonate), can be made as follows. To a solution of 2-(3-bromophenyl)-2-((4-methyl-4H-1,2,4-triazol-3-yl)methyl)propane-1,3-diol (170 mg, 0.52 mmol) in pyridine (5 mL) at 0° C. was added a solution of 4-toluenesulfonyl chloride (298.1 mg, 1.56 mmol) in pyridine (2 mL) over 30 minutes then it was stirred for 8 h. The reaction was diluted with EtOAc (50 mL) and washed with saturated aqueous NH₄Cl solution, dried over sodium sulfate, filtered and concentrated. The residue was purified by chromatography on silica gel (EtOAc in heptanes) to get 2-(3-bromophenyl)-2-((4-methyl-4H-1,2,4-triazol-3-yl)methyl)propane-1,3-diyl bis(4-methylbenzenesulfonate) (305 mg, 92% yield). LCMS (ESI) m/z: 636.0 [M+H]⁺

A sixth intermediate, 3-((3-(3-bromophenyl)thietan-3-yl)methyl)-4-methyl-4H-1,2,4-triazole, can be formed as follows. To a solution 2-(3-bromophenyl)-2-((4-methyl-4H-1,2,4-triazol-3-yl)methyl)propane-1,3-diyl bis(4-methylbenzenesulfonate) (250 mg, 0.39 mmol) in DMF (1 mL) was added potassium thioacetate (230 mg, 2.01 mmol) and cesium carbonate (256.7 mg, 0.79 mmol) and reaction mixture was heated at 110° C. for 8 h. The reaction was diluted with 10% MeOH in EtOAc (50 mL) and washed with water, dried over sodium sulfate, filtered and concentrated. The crude was purified by chromatography on silica gel (0-15% MeOH in DCM) to get 3-((3-(3-bromophenyl)thietan-3-yl)methyl)-4-methyl-4H-1,2,4-triazole (15 mg, 12% yield). LCMS (ESI) m/z: 326.0 [M+H]+

A seventh intermediate, 3-(3-bromophenyl)-3-((4-methyl-4H-1,2,4-triazol-3-yl)-methyl)thietane 1,1-dioxide, can ne made as follows. To a solution of 3-((3-(3-bromo-phenyl)thietan-3-yl)methyl)-4-methyl-4H-1,2,4-triazole (15 mg, 0.02 mmol) in DCM (1 mL) at 0° C. was added 3-chloroperbenzoic acid (m-CPBA) (31.9 mg, 0.19 mmol) and the reaction was stirred at rt for 2 h. The reaction was diluted with EtOAc (5 mL) and quenched with saturated aqueous Na₂S₂O₃ solution. Organic layer was separated and washed with brine, dried over sodium sulfate, filtered and concentrated. The crude residue was purified by chromatography on C18 silica gel (acetonitrile in ammonium formate buffer, pH=3.8). Appropriate fractions were concentrated, frozen and lyophilized to afford 3-(3-bromophenyl)-3-((4-methyl-4H-1,2,4-triazol-3-yl)methyl)thietane 1,1-dioxide (5 mg, 33% yield). LCMS (ESI) m/z: 356.0, 357.9 [M+H]+

To make Compound 242, a microwave vial equipped with a stir bar was charged with K₃PO₄ (10.8 mg, 0.05 mmol) and flame dried under nitrogen. Tert-butyl (1-methylcyclobutyl)((3-oxo-7-(trifluoromethyl)isoindolin-5-yl)methyl)carbamate (Intermediate R; 7.2 mg, 0.02 mmol), 3-(3-bromophenyl)-3-((4-methyl-4H-1,2,4-triazol-3-yl)methyl)thietane 1,1-dioxide (5 mg, 0.01 mmol), tris(dibenzylideneacetone) dipalladium(0) (0.9 mg, 0.001 mmol), Me₄tButylXphos (0.9 mg, 0.002 mmol) were then added and the vial was flushed with nitrogen for several minutes. Degassed tert-butanol (1 mL) was added via syringe, the vial was capped and the reaction mixture was stirred at 110° C. for 16 h then cooled down to rt. The reaction was diluted with water and EtOAc and mixture was passed through a pad of Celite. Organic layer was dried over sodium sulfate, filtered and evaporated. Obtained crude was dissolved in DCM (2 mL), trifluoroacetic acid (0.5 mL) was added and stirred for 1 h. Solvent was evaporated and the crude mixture was dissolved in DCM (2 mL), triethylamine (300 μL) was added and the mixture was stirred for 15 minutes. Solvent was evaporated and the crude residue was purified by chromatography on C18 silica gel (acetonitrile in 0.5% formic acid buffer). Appropriate fractions were concentrated, frozen and lyophilized to afford crude 2-(3-(3-((4-methyl-4H-1,2,4-triazol-3-yl)methyl)-1,1-dioxidothietan-3-yl)phenyl)-6-(((1-methylcyclobutyl)amino)methyl)-4-(trifluoromethyl)-isoindolin-1-one (1 mg, 12% yield). LCMS (ESI) m/z: 574.2 [M+H]⁺¹H NMR (400 MHz, CD₃CN) δ 8.02 (s, 1H), 7.99-7.95 (m, 2H), 7.89 (s, 1H), 7.40-7.34 (m, 2H), 6.78 (d, J=7.8 Hz, 1H), 4.95 (s, 2H), 4.82-4.76 (m, 2H), 4.58-4.52 (m, 2H), 3.87 (s, 2H), 3.51 (s, 2H), 2.72 (s, 3H), 2.09-1.99 (m, 2H), 1.86-1.71 (m, 4H), 1.29 (s, 3H).

### Example 55: Compound 243

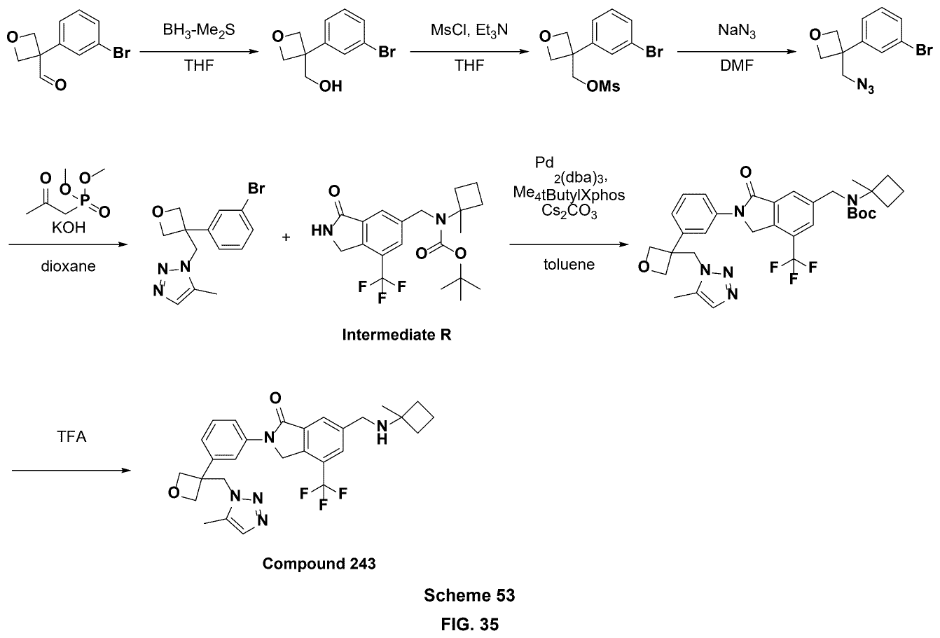

Compound 243 (2-(3-(3-((5-methyl-1H-1,2,3-triazol-1-yl)methyl)oxetan-3-yl)-phenyl)-6-(((1-methylcyclobutyl)amino)methyl)-4-(trifluoromethyl)isoindolin-1-one) can be synthesized according to Scheme 53, FIG. 35.

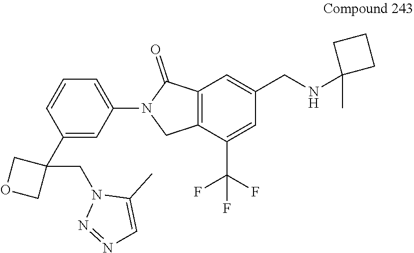 [SMILES: Cc1cnnn1CC1(c2cccc(N3Cc4c(cc(CNC5(C)CCC5)cc4C(F)(F)F)C3=O)c2)COC1]

A first intermediate, (3-(3-bromophenyl)oxetan-3-yl)methanol, can be made as follows. To a solution of 3-(3-bromophenyl)oxetane-3-carbaldehyde (1 g, 4.15 mmol) in THF (20.7 mL) at 0° C. was added a 2 M borane dimethyl sulfate complex solution in THF (3.1 mL, 6.22 mmol). The reaction was gradually warmed to rt and stirred for 30 minutes. The reaction was quenched with the addition of saturated aqueous NaHCO₃ solution (20 mL) and the product was extracted with EtOAc (2×20 mL) then with 30% IPA in CHCl₃ (2×10 mL). The combined organic layers were dried over sodium sulfate, filtered and concentrated. The crude was purified by chromatography on silica gel to afford (3-(3-bromophenyl)oxetan-3-yl)methanol (865 mg, 86% yield) as a white solid. ¹H NMR (400 MHz, CDCl₃) δ 7.51-7.37 (m, 1H), 7.34-7.18 (m, 2H), 7.09-6.93 (m, 1H), 4.93 (d, J=6.0 Hz, 2H), 4.75 (d, J=6.1 Hz, 2H), 4.05 (s, 2H).

A second intermediate, (3-(3-bromophenyl)oxetan-3-yl)methyl methanesulfonate, can be made as follows. To a solution of (3-(3-bromophenyl)oxetan-3-yl)methanol (0.87 g, 3.56 mmol) in THF (17.8 mL) at 0° C. was added methanesulfonyl chloride (0.36 mL, 4.63 mmol) then triethylamine (0.55 mL, 3.91 mmol). The reaction was warmed to rt and stirred for an additional 2 h. Water (20 mL) was added and the product was extracted with EtOAc (3×20 mL). The organic layers were combined, dried over sodium sulfate, filtered and concentrated to give (3-(3-bromophenyl)oxetan-3-yl)methyl methanesulfonate. The yield was assumed quantitative and the crude was taken to the next step without further purification. ¹H NMR (400 MHz, CDCl₃) δ 7.54-7.38 (m, 1H), 7.35-7.23 (m, 2H), 7.09-6.95 (m, 1H), 4.97 (d, J=6.4 Hz, 2H), 4.78 (d, J=6.5 Hz, 2H), 4.59 (s, 2H), 2.93-2.81 (m, 3H).

A third intermediate, 3-(azidomethyl)-3-(3-bromophenyl)oxetane, can be made as follows. To a solution of (3-(3-bromophenyl)oxetan-3-yl)methyl methanesulfonate (1.143 g, 3.56 mmol) in DMF (17.8 mL) was added sodium azide (439.7 mg, 6.76 mmol) and the reaction was heated to 80° C. for 16 h. Water (20 mL) was added and the product was extracted with DCM (3×50 mL). The combined organic layers were dried over sodium sulfate, filtered and concentrated. The crude was purified by chromatography on silica gel (0-50% EtOAc in heptanes) to afford 3-(azidomethyl)-3-(3-bromophenyl)oxetane (746 mg, 78% yield) as a light-yellow oil. ¹H NMR (400 MHz, CDCl₃) δ 7.53-7.43 (m, 1H), 7.33-7.22 (m, 1H), 7.22-7.14 (m, 1H), 7.06-6.97 (m, 1H), 4.93 (d, J=6.0 Hz, 2H), 4.69 (d, J=5.8 Hz, 2H), 3.87 (s, 2H).

A fourth intermediate, 1-((3-(3-bromophenyl)oxetan-3-yl)methyl)-5-methyl-1H-1,2,3-triazole, can be made as follows. To a solution of 3-(azidomethyl)-3-(3-bromophenyl)-oxetane (746 mg, 2.78 mmol) in 1,4-dioxane (10 mL) was added dimethyl (2-oxopropyl)-phosphonate (0.58 mL, 4.17 mmol) followed by potassium hydroxide (crushed pallets) (734.7 mg, 11.13 mmol). The reaction was heated to 80° C. for 16 h. The reaction was cooled to rt. Water (50 mL) was added and the product was extracted with DCM (3×50 mL). The combined organic layers were dried over sodium sulfate, filtered and concentrated. The crude was purified by chromatography on silica gel (0-5% MeOH in DCM) to afford 1-((3-(3-bromophenyl)oxetan-3-yl)methyl)-5-methyl-1H-1,2,3-triazole (415 mg, 48% yield) as an off-white solid. LCMS (ESI) m/z: 308.1, 310.0 [M+H]⁺.

A fifth intermediate, tert-butyl ((2-(3-(3-((5-methyl-1H-1,2,3-triazol-1-yl)methyl)-oxetan-3-yl)phenyl)-3-oxo-7-(trifluoromethyl)isoindolin-5-yl)methyl)(1-methylcyclobutyl)-carbamate, can be made as follows. A vial was charged with tert-butyl-(1-methylcyclobutyl)-((3-oxo-7-(trifluoromethyl)isoindolin-5-yl)methyl)carbamate (Intermediate R; 77.8 mg, 0.19 mmol), 1-((3-(3-bromophenyl)oxetan-3-yl)methyl)-5-methyl-1H-1,2,3-triazole (50 mg, 0.16 mmol), Me₄tButylXphos (15.6 mg, 0.03 mmol), tris(dibenzylideneacetone)dipalladium(0) (14.9 mg, 0.02 mmol) and cesium carbonate (106.4 mg, 0.32 mmol). The vial was purged with nitrogen before degassed toluene (1.6 mL) was added and the vial was sealed. The reaction mixture was stirred at 110° C. for 16 h. 3:1 CHCl₃/IPA was added and the reaction was filtered through a 0.45 μm PTFE filter. Water (3 mL) was added to the residue and the product was extracted with 3:1 CHCl₃/IPA (3×3 mL). The combined organic layers were dried over sodium sulfate, filtered, and concentrated. The crude was purified by chromatography on silica gel (0-20% MeOH in DCM) to afford tert-butyl ((2-(3-(3-((5-methyl-1H-1,2,3-triazol-1-yl)methyl)oxetan-3-yl)phenyl)-3-oxo-7-(trifluoromethyl)isoindolin-5-yl)methyl)(1-methylcyclobutyl)carbamate (57 mg, 56% yield) as a yellow solid. LCMS (ESI) m/z: 626.2 [M+H]⁺.

To make Compound 243, to a solution of tert-butyl ((2-(3-(3-((5-methyl-1H-1,2,3-triazol-1-yl)methyl)oxetan-3-yl)phenyl)-3-oxo-7-(trifluoromethyl)isoindolin-5-yl)methyl)(1-methylcyclobutyl)carbamate (57 mg, 0.09 mmol) in DCM (0.46 mL) was added trifluoroacetic acid (0.14 mL, 1.82 mmol). The reaction was stirred at rt for 2 h. Toluene was added and the excess TFA was removed. The reaction was diluted with saturated aqueous NaHCO₃ solution (20 mL) and the product was extracted with 30% IPA in CHCl3 (3×20 mL). The crude product was purified by semi-prep. LCMS 10-30% gradient of acetonitrile in ammonium formate buffer, pH=3.8). Appropriate fractions were concentrated, frozen and lyophilized to provide 2-(3-(3-((5-methyl-1H-1,2,3-triazol-1-yl)-methyl)oxetan-3-yl)phenyl)-6-(((1-methylcyclobutyl)amino)methyl)-4-(trifluoromethyl)-isoindolin-1-one (12.8 mg, 27% yield). LCMS (ESI) m/z: 526.1 [M+H]⁺. ¹H NMR (400 MHz, DMSO-d6) δ 8.27 (s, 1H), 8.05 (s, 1H), 8.01 (s, 1H), 7.94 (dd, J=8.2, 1.9 Hz, 1H), 7.34-7.26 (m, 2H), 7.22 (s, 1H), 6.60 (d, J=7.7 Hz, 1H), 5.10-5.00 (m, 4H), 4.95-4.83 (m, 4H), 3.83 (s, 2H), 2.06-1.91 (m, 2H), 1.83-1.59 (m, 4H), 1.49 (s, 3H), 1.23 (s, 3H).

### Example 56: Compound 244

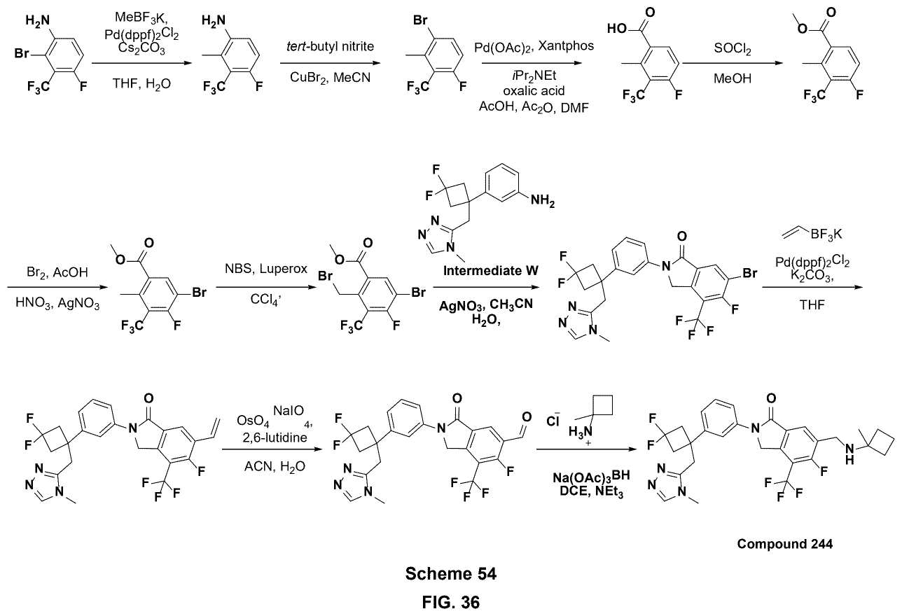

Compound 244 (2-(3-(3,3-difluoro-1-((4-methyl-4H-1,2,4-triazol-3-yl)methyl)cyclo-butyl)phenyl)-5-fluoro-6-(((1-methylcyclobutyl)amino)methyl)-4-(trifluoromethyl)isoindolin-1-one) can be synthesized according to Scheme 54, FIG. 36.

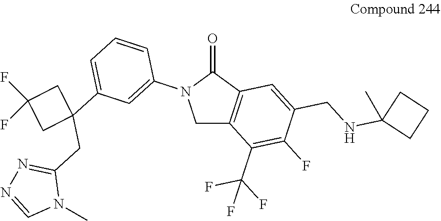 [SMILES: Cn1cnnc1CC1(c2cccc(N3Cc4c(cc(CNC5(C)CCC5)c(F)c4C(F)(F)F)C3=O)c2)CC(F)(F)C1]

A first intermediate, 4-fluoro-2-methyl-3-(trifluoromethyl)aniline, can be made as follows. A sealed tube was charged with 2-bromo-4-fluoro-3-(trifluoromethyl)aniline (5.3 g, 20.54 mmol), cesium carbonate (20.08 g, 61.63 mmol) potassium methyltrifluoroborate (5.01 g, 41.08 mmol) and Pd(dppf)₂Cl₂ (1.5 g, 2.05 mmol). The reaction was placed under vacuum and then purged with nitrogen three times. Degassed THF (49.2 mL) and water (4.9 mL) was then added. The vial was sealed and heated at 100° C. for 16 h. The reaction was cooled down, water (150 mL) was added and the product was extracted with EtOAc (1×150 mL). Organic layer was washed with water (1×150 mL) and then dried over sodium sulfate, filtered and concentrated. The crude was purified by chromatography on silica gel (0-100% EtOAc in heptanes) to obtain 4-fluoro-2-methyl-3-(trifluoromethyl)aniline (3.4 g, 86% yield) was isolated as a dark-red needles. LCMS (ESI) m/z: 193.2 [M+H]⁺ (Br pattern).

A second intermediate, 1-bromo-4-fluoro-2-methyl-3-(trifluoromethyl)benzene, can be made as follows. To a suspension of 4-fluoro-2-methyl-3-(trifluoromethyl)aniline (3.45 g, 17.86 mmol) and copper(II) bromide (3.99 g, 17.86 mmol) in MeCN (59.5 mL) was added tert-butyl nitrite (2.34 mL, 19.65 mmol) at 0° C. The reaction was gradually warmed to rt and stirred for 16 h. Water (100 mL) was added to the reaction and the product was extracted with EtOAc (3×100 mL). The combined organic layers were dried over sodium sulfate, filtered and concentrated. The crude was purified by chromatography on silica gel (0-10% EtOAc in pentane) to obtain 1-bromo-4-fluoro-2-methyl-3-(trifluoromethyl)benzene (3.77 g, 82% yield) as a dark-red oil. In this instance, caution is required as the product is volatile. ¹H NMR (400 MHz, CDCl₃) δ 7.70 (dd, J=8.9, 4.9 Hz, 1H), 7.00-6.84 (m, 1H), 2.57 (q, J=2.1 Hz, 3H).

A third intermediate, 4-fluoro-2-methyl-3-(trifluoromethyl)benzoic acid, can be made as follows. A pressure vessel was charged with 1-bromo-4-fluoro-2-methyl-3-(trifluoromethyl)benzene (3.77 g, 14.67 mmol), palladium acetate (0.33 g, 1.47 mmol), XantPhos (0.85 g, 1.47 mmol) and oxalic acid (2.0 mL, 22 mmol). After purging with nitrogen, degassed DMF (48.9 mL) containing DIPEA (3.8 mL, 22 mmol) and acetic anhydride (2.08 mL, 22 mmol) were added and the reaction was heated to 100° C. for 16 h. The reaction was cooled down to rt, 1 M NaOH solution was added and the aqueous layer was washed with Et₂O (2×100 mL). The organic wash was back extracted with NaOH [1M, 150 mL]. The aqueous layer was acidified 6 M HCl solution and the product was extracted with DCM (2×400 mL). The combined organic layers were dried over sodium sulfate, filtered and concentrated to afford 4-fluoro-2-methyl-3-(trifluoromethyl)benzoic acid (2.2 g, 68% yield) as an orange solid. LCMS (ESI) m/z: 221.4 [M+H]⁺. ¹H NMR (400 MHz, CDCl₃) δ 8.10 (dd, J=8.9, 5.4 Hz, 1H), 7.12-7.06 (m, 1H), 2.74 (q, J=2.3 Hz, 3H).

A fourth intermediate, methyl 4-fluoro-2-methyl-3-(trifluoromethyl)benzoate, can be made as follows. To a solution of 4-fluoro-2-methyl-3-(trifluoromethyl)benzoic acid (2.2 g, 9.9 mmol), in methanol (24.8 mL) was added thionyl chloride (1.81 mL, 24.76 mmol) dropwise at 0° C. The reaction mixture was warmed to rt and then heated to 50° C. and stirred for 16 h. The reaction mixture was concentrated under reduced pressure. The crude reaction mixture was re-dissolved in water (60 mL) and the pH was adjusted to 8.5 using saturated aqueous NaHCO₃ solution. The solution was extracted with DCM (3×100 mL) and the combined organic layers were dried over sodium sulfate, filtered and concentrated to afford methyl 4-fluoro-2-methyl-3-(trifluoromethyl)benzoate (1.770 g, 77% yield) as a yellow oil.

A fifth intermediate, methyl 5-bromo-4-fluoro-2-methyl-3-(trifluoromethyl)benzoate, can be made as follows. To a solution of methyl 4-fluoro-2-methyl-3-(trifluoromethyl)-benzoate (1.77 g, 7.49 mmol) in acetic acid (20 mL) were added HNO₃, 70% in water (3.35 mL, 74.95 mmol) and bromine (0.42 mL, 8.24 mmol) followed by a slow dropwise addition of AgNO₃, 2,5M in water (3.9 mL, 9.74 mmol). The mixture was then stirred at rt for 16 h. LCMS analysis showed not a complete conversion. Bromine (0.42 mL, 8.24 mmol), AgNO₃, 2.5M in water (3.9 mL, 9.74 mmol), HNO₃, 70% in water (3.35 mL, 74.95 mmol) were added and the reaction was stirred for an additional 1.5 h. The reaction mixture was then poured on ice, diluted with saturated aqueous NaHCO₃ and the pH was adjusted to 12 using 1 M NaOH solution. The product was extracted with DCM (3×400 mL). The combined organic phases were dried over sodium sulfate, filtered and concentrated. The crude was purified by chromatography on silica gel (0-50% EtOAc in heptanes) to obtain methyl 5-bromo-4-fluoro-2-methyl-3-(trifluoromethyl)benzoate (1.700 g, 72% yield) as a beige solid. ¹H NMR (400 MHz, CDCl₃) δ 8.15 (d, J=6.7 Hz, 1H), 3.92 (s, 3H), 2.62 (q, J=2.3 Hz, 3H).

A sixth intermediate, methyl 5-bromo-2-(bromomethyl)-4-fluoro-3-(trifluoromethyl)-benzoate, can be made as follows. To a solution of methyl 5-bromo-4-fluoro-2-methyl-3-(trifluoromethyl)benzoate (500 mg, 1.59 mmol) in carbon tetrachloride (10.9 mL) was added N-bromosuccinimide (423.7 mg, 2.38 mmol) and benzoyl peroxide (38.4 mg, 0.16 mmol). The reaction was stirred at 80° C. for 16 h and then quenched with the addition of saturated aqueous NaHCO₃ solution (30 mL) and the product was extracted with DCM (3×). The combined organic layers were dried over sodium sulfate, filtered and concentrated. The crude was purified by chromatography on silica gel (0-50% EtOAc in heptanes) to obtain methyl 5-bromo-2-(bromomethyl)-4-fluoro-3-(trifluoromethyl)benzoate (610 mg, 98% yield) as a light yellow oil. ¹H NMR (400 MHz, CDCl₃) δ 8.28 (d, J=6.6 Hz, 1H), 5.09 (s, 2H), 3.98 (s, J=0.4 Hz, 3H).

A seventh intermediate, 6-bromo-2-(3-(3,3-difluoro-1-((4-methyl-4H-1,2,4-triazol-3-yl)methyl)cyclobutyl)phenyl)-5-fluoro-4-(trifluoromethyl)isoindolin-1-one, can be made as follows. A mixture of 3-(3,3-difluoro-1-((4-methyl-4H-1,2,4-triazol-3-yl)methyl)cyclobutyl)-aniline (Intermediate W; 0.35 g, 1.24 mmol), methyl 5-bromo-2-(bromomethyl)-4-fluoro-3-(trifluoromethyl)benzoate (0.49 g, 1.24 mmol) in MeCN (12.3 mL) and water (5.4 mL) was cooled to 0° C. Silver nitrate (273.8 mg, 1.61 mmol) dissolved in water (5 mL) was added dropwise. The reaction was stirred for 16 h at rt. The reaction was diluted with saturated aqueous NaHCO₃ (70 mL) and the product was extracted with 30% IPA in CHCl₃ (3×70 mL). The combined organic layers were dried over sodium sulfate, filtered and concentrated. The crude was purified by chromatography on silica gel (0-5% MeOH in DCM) to obtain 6-bromo-2-(3-(3,3-difluoro-1-((4-methyl-4H-1,2,4-triazol-3-yl)methyl)cyclo-butyl)phenyl)-5-fluoro-4-(trifluoromethyl)isoindolin-1-one (0.344 g, 50% yield) as a yellow solid. LCMS (ESI) m/z: 559.4 [M+H]⁺ (Br Pattern).

An eighth intermediate, 2-(3-(3,3-difluoro-1-((4-methyl-4H-1,2,4-triazol-3-yl)-methyl)cyclobutyl)phenyl)-5-fluoro-4-(trifluoromethyl)-6-vinylisoindolin-1-one, can be made as follows. A screw-cap vial was charged with 6-bromo-2-(3-(3,3-difluoro-1-((4-methyl-4H-1,2,4-triazol-3-yl)methyl)cyclobutyl)phenyl)-5-fluoro-4-(trifluoromethyl)isoindolin-1-one (100 mg, 0.18 mmol), potassium carbonate (123.6 mg, 0.8900 mmol), 1,1-bis(diphenyl-phosphino)ferrocene-palladium dichloride (13.3 mg, 0.02 mmol) and potassium vinyl-trifluoroborate (47.9 mg, 0.36 mmol) degassed THF (0.89 mL) was added and the tube was sealed. The reaction mixture was heated in an oil bath at 95° C. for 16 h. Water (5 mL) was added and the product was extracted with 30% IPA in CHCl₃ (3×5 mL). The organic layers were combined, dried over sodium sulfate, filtered and concentrated. The crude was purified by chromatography on silica gel (0-5% MeOH in DCM) to obtain 2-(3-(3,3-difluoro-1-((4-methyl-4H-1,2,4-triazol-3-yl)methyl)cyclobutyl)phenyl)-5-fluoro-4-(trifluoromethyl)-6-vinylisoindolin-1-one (71 mg, 78% yield) a brown-orange solid. LCMS (ESI) m/z: 507.4 [M+H]⁺.

A ninth intermediate, 2-(3-(3,3-difluoro-1-((4-methyl-4H-1,2,4-triazol-3-yl)-methyl)cyclobutyl)phenyl)-6-fluoro-3-oxo-7-(trifluoromethyl)isoindoline-5-carbaldehyde can be made as follows. To a solution of 2-(3-(3,3-difluoro-1-((4-methyl-4H-1,2,4-triazol-3-yl)methyl)cyclobutyl) phenyl)-5-fluoro-4-(trifluoromethyl)-6-vinylisoindolin-1-one (40 mg, 0.08 mmol) in MeCN (2.5 mL) was added 2,6-lutidine (14.62 uL, 0.13 mmol), NalO₄ (54.0 mg, 0.25 mmol), water (0.28 mL) and OSO₄, 4% solution in water (158.6 uL, 0.02 mmol) at 0° C. The reaction was gradually warmed to rt. LCMS analysis after 16 h showed a full conversion to the diol intermediate (m/z: 541) and ˜30% of the aldehyde. The reaction was quenched with the addition of water (5 mL) and the product was extracted using 30% IPA in CHCl₃ (3×5 mL). The organic layers were combined, dried over sodium sulfate, filtered and concentrated. The crude was dissolved in MeCN: H₂O (4 mL: 1 mL) and NalO₄ (200 mg) was added. The reaction was stirred at rt for 16 h. The above workup was repeated (10 mL for each phase) to 2-(3-(3,3-difluoro-1-((4-methyl-4H-1,2,4-triazol-3-yl)methyl)cyclo-butyl)phenyl)-6-fluoro-3-oxo-7-(trifluoromethyl)isoindoline-5-carbaldehyde (30 mg, 75% yield) as a beige solid which was used to the next step without further purification. LCMS (ESI) m/z: 509.3 [M+H]⁺.

To make Compound 244, to a stirred solution of 2-(3-(3,3-difluoro-1-((4-methyl-4H-1,2,4-triazol-3-yl)methyl)cyclobutyl)phenyl)-6-fluoro-3-oxo-7-(trifluoromethyl)isoindoline-5-carbaldehyde (30 mg, 0.06 mmol) and 1-methylcyclobutanamine; hydrochloride (21.5 mg, 0.18 mmol) in DCE (1 mL) was added triethylamine (24.7 μL, 0.18 mmol) followed by sodium triacetoxyborohydride (125.1 mg, 0.59 mmol) and the suspension was stirred at rt for 16 h. The reaction was quenched with addition of water (3 drops) and concentrated. The crude residue was purified by chromatography on C18 silica gel (0-100% acetonitrile in ammonium formate buffer, pH=3.8). Appropriate fractions were concentrated to afford the product only 62% pure. A second purification was carried out by chromatography on C18 silica gel (0-100% acetonitrile in ammonium bicarbonate buffer, pH=10). Appropriate fractions were concentrated, frozen and lyophilized to afford 2-(3-(3,3-difluoro-1-((4-methyl-4H-1,2,4-triazol-3-yl)methyl)cyclobutyl)phenyl)-5-fluoro-6-(((1-methylcyclobutyl)amino)-methyl)-4-(trifluoromethyl)isoindolin-1-one (5.9 mg, 17% yield). LCMS (ESI) m/z: 578.4 [M+H]+. ¹H NMR (400 MHz, DMSO-d6) δ 8.24 (d, J=6.2 Hz, 1H), 8.18 (s, 1H), 7.87 (d, J=8.3 Hz, 1H), 7.41-7.22 (m, 2H), 6.80 (d, J=8.0 Hz, 1H), 5.13 (s, 2H), 3.79 (d, J=7.9 Hz, 2H), 3.28-3.17 (m, 4H), 3.11-2.91 (m, 2H), 2.72 (s, 3H), 2.05-1.87 (m, 2H), 1.77-1.59 (m, 4H), 1.24 (s, 3H).

### Example 57: Compound 245 and Compound 246

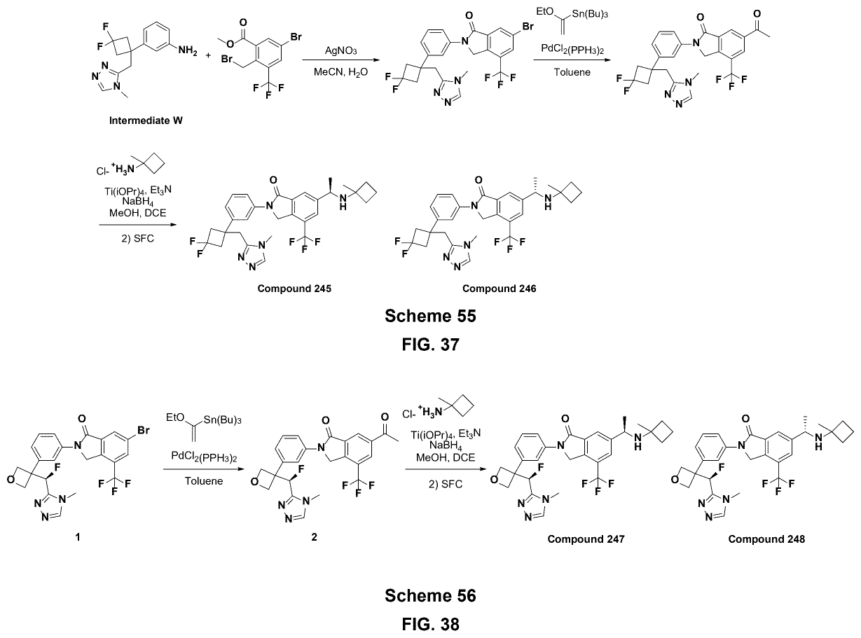

Compounds 245 ((R)-2-(3-(3,3-difluoro-1-((4-methyl-4H-1,2,4-triazol-3-yl)-methyl)cyclobutyl)phenyl)-6-(1-((1-methylcyclobutyl)amino)ethyl)-4-(trifluoromethyl)-isoindolin-1-one) and 246 ((S)-2-(3-(3,3-difluoro-1-((4-methyl-4H-1,2,4-triazol-3-yl)methyl)-cyclobutyl)phenyl)-6-(1-((1-methylcyclobutyl)amino)ethyl)-4-(trifluoromethyl)isoindolin-1-one) can be synthesized according to Scheme 55, FIG. 37.

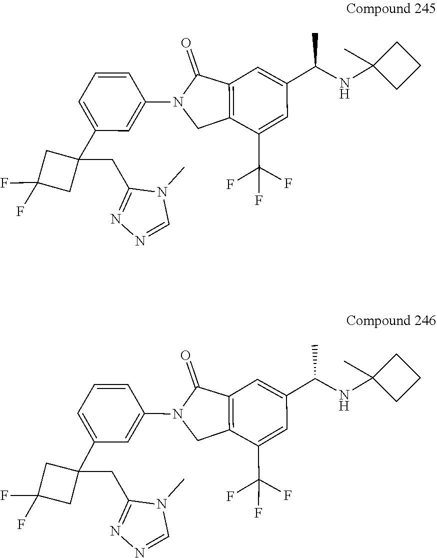 [SMILES: C[C@@H](NC1(C)CCC1)c1cc2c(c(C(F)(F)F)c1)CN(c1cccc(C3(Cc4nncn4C)CC(F)(F)C3)c1)C2=O ; C[C@H](NC1(C)CCC1)c1cc2c(c(C(F)(F)F)c1)CN(c1cccc(C3(Cc4nncn4C)CC(F)(F)C3)c1)C2=O]

A first intermediate, 6-bromo-2-(3-(3,3-difluoro-1-((4-methyl-4H-1,2,4-triazol-3-yl)methyl)cyclobutyl)phenyl)-4-(trifluoromethyl)isoindolin-1-one, can be synthesized as follows.

A mixture of 3-(3,3-difluoro-1-((4-methyl-4H-1,2,4-triazol-3-yl)methyl)cyclobutyl)-aniline (Intermediate W; 230 mg, 0.83 mmol) and methyl 5-bromo-2-(bromomethyl)-3-(trifluoromethyl)benzoate (310.7 mg, 0.83 mmol) in MeCN (10 mL) and water (5 mL) was cooled to 0° C., then a solution of silver nitrate (182.5 mg, 1.07 mmol) in water (5 mL) was added dropwise. The reaction was stirred for 17 h at rt. Saturated aqueous NaHCO₃ was added until the solution reached pH 8. The mixture was diluted with ACN, filtered through Celite, and the cake was washed with a 9:1 mixture of DCM:MeOH. The filtrate was washed with brine, dried over sodium sulfate filtered and concentrated. The crude was purified by chromatography on silica gel (0-8% MeOH in DCM) to obtain 6-bromo-2-(3-(3,3-difluoro-1-((4-methyl-4H-1,2,4-triazol-3-yl)methyl)cyclobutyl)phenyl) −4-(trifluoromethyl)-isoindolin-1-one (170 mg, 38% yield). LCMS (ESI) m/z: 542.9 [M+H]⁺.

A second intermediate, 6-acetyl-2-(3-(3,3-difluoro-1-((4-methyl-4H-1,2,4-triazol-3-yl)methyl)cyclobutyl)phenyl)-4-(trifluoromethyl)isoindolin-1-one, can be synthesized as follows. A flask was charged with 6-bromo-2-(3-(3,3-difluoro-1-((4-methyl-4H-1,2,4-triazol-3-yl)methyl)cyclobutyl)phenyl)-4-(trifluoromethyl)isoindolin-1-one (85 mg, 0.16 mmol) and dichlorobis(triphenylphosphine)palladium (II) (11.0 mg, 0.00 mmol). Degassed toluene (517.3 uL) was added followed by tributyl(1-ethoxyvinyl)tin (79.6 uL, 0.24 mmol). The reaction was heated to 100° C. for 16 h. The reaction was cooled to rt 1 N HCl solution (1 mL) was added and the reaction was stirred at rt for 20 minutes. The reaction was diluted with saturated aqueous NaHCO₃ solution, extracted with EtOAc, dried over sodium sulfate, filtered and evaporated. The crude was purified by chromatography on silica gel (0-15% MeOH in DCM) to obtain 6-acetyl-2-(3-(3,3-difluoro-1-((4-methyl-4H-1,2,4-triazol-3-yl)methyl)cyclobutyl)phenyl)-4-(trifluoromethyl)isoindolin-1-one (53.6 mg, 68% yield). LCMS (ESI) m/z: 505.2 [M+H]⁺.

To obtain Compounds 245 and 246, a solution of 6-acetyl-2-(3-(3,3-difluoro-1-((4-methyl-4H-1,2,4-triazol-3-yl)methyl)cyclobutyl) phenyl)-4-(trifluoromethyl)isoindolin-1-one (53 mg, 0.11 mmol) and 1-methylcyclobutanamine hydrochloride (25.6 mg, 0.21 mmol) in DCE (0.52 mL) was treated with triethylamine (29.3 uL, 0.21 mmol) and titanium isopropoxide (159.3 uL, 0.53 mmol). The reaction was stirred at 80° C. for 16 h. The reaction was cooled to rt and methanol (531.5 μL) was added and the reaction was cooled to 0° C. before sodium borohydride (19.9 mg, 0.53 mmol) was added slowly. After 30 minutes at rt, the reaction was diluted with saturated aqueous NaHCO₃ solution, extracted with EtOAc, dried over sodium sulfate, filtered and evaporated. The crude product was purified by semi-prep. LCMS 40-60% gradient of acetonitrile in ammonium bicarbonate buffer, pH=10). Appropriate fractions were concentrated, frozen and lyophilized to provide a mixture of enantiomers.

The above racemate was further purified by chiral SFC (Column=IC; Column dimensions=250 mm×10 mm×5 μm; Detection wavelength=310 nm; Flow rate=10 mL/min; Run time=30 min; Column temperature=40° C.) with 35% IPA+10 mM AmF/carbon dioxide) to afford two products, characterized as follows.

(R)-2-(3-(3,3-difluoro-1-((4-methyl-4H-1,2,4-triazol-3-yl)methyl)cyclobutyl)phenyl)-6-(1-((1-methylcyclobutyl)amino)ethyl)-4-(trifluoromethyl)isoindolin-1-one (peak 1) (9.8 mg, 16% yield), LCMS (ESI) m/z: 574.5 [M+H]+. ¹H NMR (400 MHz, DMSO-d6) δ 8.17 (s, 1H), 8.10 (d, J=11.3 Hz, 2H), 7.90 (d, J=8.0 Hz, 1H), 7.39-7.30 (m, 2H), 6.78 (d, J=7.9 Hz, 1H), 5.07 (s, 2H), 4.13-4.04 (m, 1H), 4.01 (s, 1H), 3.24 (s, 3H), 3.05-2.96 (m, 2H), 2.71 (s, 3H), 2.33-2.26 (m, 1H), 1.91-1.83 (m, 1H), 1.83-1.66 (m, 3H), 1.59-1.46 (m, 3H), 1.44-1.36 (m, 1H), 1.06 (s, 3H), 0.91 (d, J=6.7 Hz, 1H).

(S)-2-(3-(3,3-difluoro-1-((4-methyl-4H-1,2,4-triazol-3-yl)methyl)cyclobutyl)phenyl)-6-(1-((1-methylcyclobutyl)amino)ethyl)-4-(trifluoromethyl)isoindolin-1-one (peak 2) (5.8 mg, 10% yield), LCMS (ESI) m/z: 574.4 [M+H]⁺. ¹H NMR (400 MHz, DMSO-d6) δ 8.17 (s, 1H), 8.10 (d, J=11.3 Hz, 2H), 7.90 (dd, J=8.2, 1.3 Hz, 1H), 7.38-7.31 (m, 2H), 6.78 (d, J=8.1 Hz, 1H), 5.06 (s, 2H), 4.13-4.05 (m, 1H), 4.01 (s, 1H), 3.24 (s, 3H), 3.06-2.96 (m, 2H), 2.71 (s, 3H), 2.31-2.26 (m, 2H), 1.91-1.83 (m, 1H), 1.83-1.66 (m, 3H), 1.57-1.48 (m, 3H), 1.44-1.37 (m, 1H), 1.06 (s, 3H), 0.91 (d, J=6.7 Hz, 1H).

### Example 58: Compound 247 and Compound 248

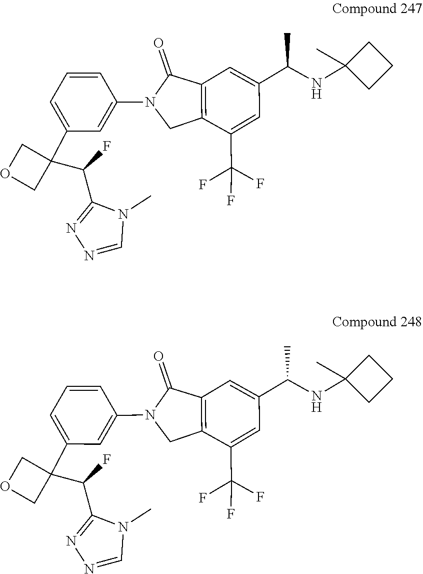 [SMILES: C[C@@H](NC1(C)CCC1)c1cc2c(c(C(F)(F)F)c1)CN(c1cccc(C3([C@@H](F)c4nncn4C)COC3)c1)C2=O ; C[C@H](NC1(C)CCC1)c1cc2c(c(C(F)(F)F)c1)CN(c1cccc(C3([C@@H](F)c4nncn4C)COC3)c1)C2=O]

Compounds 247 (2-(3-(3-((R)-fluoro(4-methyl-4H-1,2,4-triazol-3-yl)methyl)oxetan-3-yl)phenyl)-6-((R)-1-((1-methylcyclobutyl)amino)ethyl)-4-(trifluoromethyl)isoindolin-1-one) and 248 (2-(3-(3-((R)-fluoro(4-methyl-4H-1,2,4-triazol-3-yl)methyl)oxetan-3-yl)phenyl)-6-((S)-1-((1-methylcyclobutyl)amino)ethyl)-4-(trifluoromethyl)isoindolin-1-one) can be synthesized according to Scheme 56, FIG. 38.

A first intermediate, (R)-6-acetyl-2-(3-(3-(fluoro(4-methyl-4H-1,2,4-triazol-3-yl)-methyl)oxetan-3-yl)phenyl)-4-(trifluoromethyl)isoindolin-1-one, can be synthesized as follows. A flask was charged with (R)-6-acetyl-2-(3-(3-(fluoro(4-methyl-4H-1,2,4-triazol-3-yl)methyl)oxetan-3-yl)phenyl)-4-(trifluoromethyl)isoindolin-1-one (170 mg, 0.32 mmol) and dichlorobis(triphenylphosphine)palladium (II) (22.7 mg, 0.03 mmol). Degassed toluene (1.6 mL) was added followed by tributyl(1-ethoxyvinyl)tin (164 uL, 0.49 mmol). The reaction was heated to 100° C. for 16 h. Water (3 mL) was added and the product was extracted with 30% IPA in CHCl₃ (3×3 mL). The organic layers were combined, dried over sodium sulfate, filtered and concentrated. The crude reaction mixture was diluted with DCM (1 mL) and trifluoroacetic acid (0.5 mL) was added. The reaction was stirred at rt for 30 min. Toluene was added and TFA was removed under reduced pressure. The crude was dissolved in DCM (5 mL) and basified using saturated aqueous NaHCO₃ solution, the product was extracted with 30% IPA in CHCl₃ (3×3 mL). The combined organic layers were dried over sodium sulfate, filtered and concentrated. The crude was purified by chromatography on silica gel (0-5% MeOH in DCM) to obtain (R)-6-acetyl-2-(3-(3-(fluoro(4-methyl-4H-1,2,4-triazol-3-yl)methyl)oxetan-3-yl)phenyl)-4-(trifluoromethyl)isoindolin-1-one (35 mg, 22% yield) as an off-white solid. LCMS (ESI) m/z: 489.1 [M+H]⁺.

A solution of (R)-6-acetyl-2-(3-(3-(fluoro(4-methyl-4H-1,2,4-triazol-3-yl)methyl)-oxetan-3-yl)phenyl)-4-(trifluoromethyl)isoindolin-1-one (50 mg, 0.10 mmol) and 1-methylcyclobutanamine hydrochloride (49.8 mg, 0.41 mmol) in DCE (0.5 mL) was treated with triethylamine (57.1 μL, 0.41 mmol) titanium isopropoxide (155 μL, 0.51 mmol) was then added and the mixture was stirred at 80° C. for 16 h. The reaction was cooled to rt and methanol (1 mL) was then added. The reaction was cooled to 0° C. and sodium borohydride (38.7 mg, 1.02 mmol) was added in 3 portions. The reaction was stirred for an additional 1 h. Water was added and the product was extracted with 30% IPA in CHCl₃ (3×5 mL). The organic layers were combined, dried over sodium sulfate, and concentrated. The crude product was purified by semi-prep. LCMS (30-70% gradient of acetonitrile in ammonium formate buffer, pH=3.8). Appropriate fractions were concentrated, frozen and lyophilized to provide a racemate (31 mg, 54% yield) as a white solid.

The above racemate was further purified by chiral SFC (Column=IC; Column dimensions=250 mm×10 mm×5 μm; Detection wavelength=310 nm; Flow rate=10 mL/min; Run time=23 min; Column temperature=40° C.) with 40% MeOH 10 mM AmF/carbon dioxide) to afford the two isomers, characterized as follows:

2-(3-(3-((R)-fluoro(4-methyl-4H-1,2,4-triazol-3-yl)methyl)oxetan-3-yl)phenyl)-6-((R)-1-((1-methylcyclobutyl)amino)ethyl)-4-(trifluoromethyl)isoindolin-1-one (peak 1) (4.6 mg, 8% yield) LCMS (ESI) m/z: 558.3 [M+H]⁺. ¹H NMR (400 MHz, DMSO-d6) δ 8.32 (s, 1H), 8.09 (s, 1H), 8.06 (s, 1H), 7.90 (dd, J=8.2, 1.4 Hz, 1H), 7.50 (t, J=1.6 Hz, 1H), 7.35 (t, J=8.0 Hz, 1H), 6.94 (d, J=7.5 Hz, 1H), 6.25 (d, J=45.8 Hz, 1H), 5.34 (d, J=6.5 Hz, 1H), 5.19 (d, J=5.9 Hz, 1H), 5.13-4.98 (m, 3H), 4.80 (dd, J=6.1, 4.0 Hz, 1H), 4.07 (q, J=6.7 Hz, 1H), 3.98 (br s, 1H), 3.14 (s, 3H), 2.29-2.21 (m, 1H), 1.89-1.64 (m, 2H), 1.60-1.43 (m, 2H), 1.43-1.34 (m, 1H), 1.25 (d, J=6.7 Hz, 3H), 1.03 (s, 3H).

2-(3-(3-((R)-fluoro(4-methyl-4H-1,2,4-triazol-3-yl)methyl)oxetan-3-yl)phenyl)-6-((S)-1-((1-methylcyclobutyl)amino)ethyl)-4-(trifluoromethyl)isoindolin-1-one (peak 2) (5.8 mg, 10% yield) LCMS (ESI) m/z: 558.3 [M+H]⁺. ¹H NMR (400 MHz, DMSO-d6) δ 8.35 (s, 1H), 8.11 (s, 1H), 8.08 (s, 1H), 7.93 (d, J=8.2 Hz, 1H), 7.53 (s,1H), 7.37 (t, J=8.0 Hz, 1H), 6.96 (d, J=7.8 Hz, 1H), 6.27 (d, J=45.8 Hz, 1H), 5.37 (d, J=6.6 Hz, 1H), 5.21 (d, J=6.1 Hz, 1H), 5.17-5.04 (m, 2H), 4.83 (dd, J=6.0, 4.0 Hz, 1H), 4.15-4.03 (m, 1H), 4.01 (s,1H), 3.17 (s, 3H), 2.34-2.22 (m, 1H), 1.90-1.67 (m, 2H), 1.60-1.37 (m, 2H), 1.30-1.20 (m, 2H), 1.06 (s, 3H), 0.88-0.81 (m, 3H).

### Example 59: Compound 249

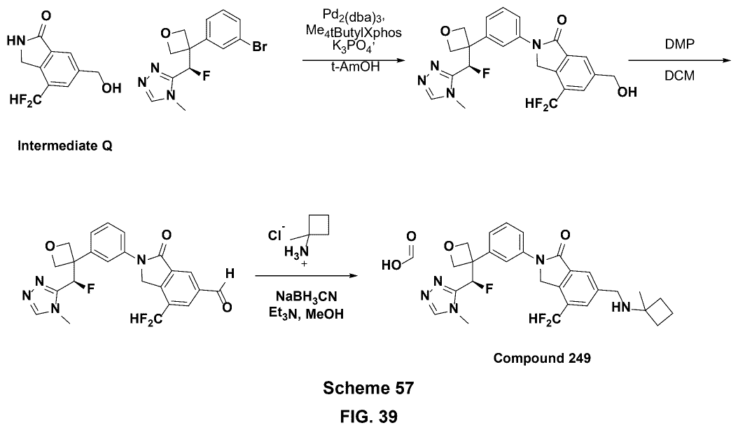

Compound 249 ((R)-4-(difluoromethyl)-2-(3-(3-(fluoro(4-methyl-4H-1,2,4-triazol-3-yl)methyl)oxetan-3-yl)phenyl)-6-(((1-methylcyclobutyl)amino)methyl)isoindolin-1-one formate) can be synthesized according to Scheme 57, FIG. 39.

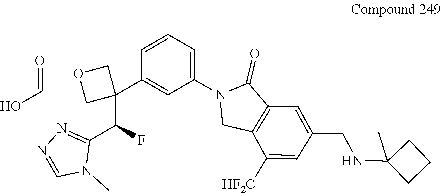

A first intermediate, (R)-4-(difluoromethyl)-2-(3-(3-(fluoro(4-methyl-4H-1,2,4-triazol-3-yl)methyl)oxetan-3-yl)phenyl)-6-(hydroxymethyl)isoindolin-1-one, can be synthesized as follows. A vial was charged with 4-(difluoromethyl)-6-(hydroxymethyl)-isoindolin-1-one (Intermediate Q; 46.7 mg, 0.22 mmol), (R)-3-((3-(3-bromophenyl)oxetan-3-yl)fluoromethyl)-4-methyl-4H-1,2,4-triazole (65 mg, 0.20 mmol), Me₄tButylXphos (19.2 mg, 0.04 mmol), tris(dibenzylideneacetone)dipalladium(0) (14.6 mg, 0.02 mmol), K₃PO₄ (84.6 mg, 0.40 mmol) and 4 Å molecular sieve sieve (note that base and MS were flame dried prior to addition). The vial was purged with nitrogen before degassed t-AmOH (1.3 mL, 0.20 mmol) was added and the vial was sealed. The reaction mixture was stirred at 110° C. for 15 h. The reaction was purified by chromatography on silica gel (2-15% MeOH in DCM) to afford (R)-4-(difluoromethyl)-2-(3-(3-(fluoro(4-methyl-4H-1,2,4-triazol-3-yl)methyl)oxetan-3-yl)phenyl)-6-(hydroxymethyl)isoindolin-1-one (57.2 mg, 63% yield). LCMS (ESI) m/z: 459.1 [M+H]⁺.

A second intermediate, (R)-7-(difluoromethyl)-2-(3-(3-(fluoro(4-methyl-4H-1,2,4-triazol-3-yl)methyl)oxetan-3-yl)phenyl)-3-oxoisoindoline-5-carbaldehyde, can be synthesized as follows. Dess-Martin periodinane (“DMP”; 101.8 mg, 0.24 mmol) was added to a suspension of (R)-4-(difluoromethyl)-2-(3-(3-(fluoro(4-methyl-4H-1,2,4-triazol-3-yl)methyl)oxetan-3-yl)phenyl)-6-(hydroxymethyl)isoindolin-1-one (55 mg, 0.12 mmol) in DCM (0.8 mL) at 0° C. The resulting mixture was allowed to warm to rt and stirred for the weekend. The reaction was quenched with saturated aqueous Na₂S₂O₃ and saturated aqueous NaHCO₃ solution and the resulting mixture was stirred vigorously for 30 minutes. The layers were separated, the aqueous phase was extracted with 4:1 CHCl₃/IPA (3×), the combined organic phases were dried over magnesium sulfate, filtered and concentrated under reduced pressure to afford (R)-7-(difluoromethyl)-2-(3-(3-(fluoro(4-methyl-4H-1,2,4-triazol-3-yl)methyl)oxetan-3-yl)phenyl)-3-oxoisoindoline-5-carbaldehyde (52.5 mg, 96% yield) as crude used as such for in the next step. LCMS (ESI) m/z: 457.1 [M+H]⁺.

To produce Compound 249, a microwave vial was charged with (R)-7-(difluoro-methyl)-2-(3-(3-(fluoro(4-methyl-4H-1,2,4-triazol-3-yl)methyl)oxetan-3-yl)phenyl)-3-oxo-isoindoline-5-carbaldehyde (52 mg, 0.11 mmol), triethylamine (95.3 μL, 0.68 mmol) and 1-methylcyclobutanamine; hydrochloride (69.3 mg, 0.57 mmol) in methanol (0.95 mL). The reaction was heated in a microwave oven for 3 minutes at 100° C. The reaction was cooled to rt and sodium cyanoborohydride (21.5 mg, 0.34 mmol) was added. The reaction was heated in a microwave oven at 100° C. for 60 minutes. The reaction was diluted with saturated aqueous NaHCO₃ solution, extracted with EtOAc, dried over sodium sulfate, filtered and evaporated. The organic layers were combined, dried over sodium sulfate, and concentrated. The crude product was purified by semi-prep. LCMS (10-30% gradient of acetonitrile in ammonium formate buffer, pH=3.8). Appropriate fractions were concentrated, frozen and lyophilized to provide (R)-4-(difluoromethyl)-2-(3-(3-(fluoro(4-methyl-4H-1,2,4-triazol-3-yl)methyl)oxetan-3-yl)phenyl)-6-(((1-methylcyclobutyl)amino)-methyl)isoindolin-1-one (18.9 mg, 32% yield). LCMS (ESI) m/z: 526.1 [M+H]⁺. ¹H NMR (400 MHz, DMSO-d6) δ 8.36 (s, 1H), 8.29 (s, 1H), 7.95 (dd, J=8.2, 1.5 Hz, 1H), 7.89 (d, J=25.2 Hz, 2H), 7.56 (s, 1H), 7.37 (t, J=8.0 Hz, 1H), 7.27 (t, J=55.0 Hz, 1H), 6.95 (d, J=7.8 Hz, 1H), 6.28 (d, J=45.9 Hz, 1H), 5.37 (d, J=6.6 Hz, 1H), 5.21 (d, J=5.9 Hz, 1H), 5.13 (dd, J=6.9, 1.7 Hz, 1H), 5.08-5.00 (m, 2H), 4.83 (dd, J=6.1, 4.0 Hz, 1H), 3.79 (s, 2H), 3.19 (s, 3H), 2.05-1.94 (m, 2H), 1.77-1.62 (m, 4H), 1.24 (s, 3H).

### Example 60: Compound 250

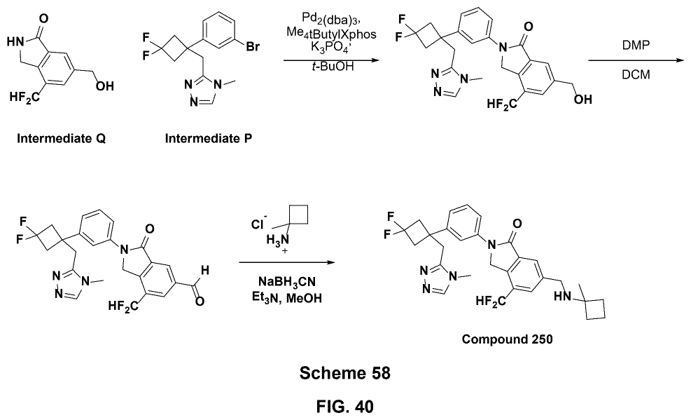

Compound 250 (2-(3-(3,3-difluoro-1-((4-methyl-4H-1,2,4-triazol-3-yl)methyl)cyclo-butyl)phenyl)-4-(difluoromethyl)-6-(((1-methylcyclobutyl)amino)methyl)isoindolin-1-one) can be synthesized according to Scheme 58, FIG. 40.

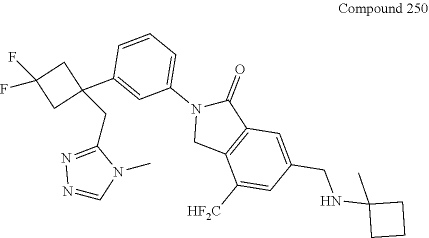

A first intermediate, 2-(3-(3,3-difluoro-1-((4-methyl-4H-1,2,4-triazol-3-yl)methyl)cyclobutyl)phenyl)-4-(difluoromethyl)-6-(hydroxymethyl)isoindolin-1-one, can be synthesized as follows. A vial was charged with 4-(difluoromethyl)-6-(hydroxymethyl)-isoindolin-1-one (Intermediate Q; 44.5 mg, 0.21 mmol), 3-((1-(3-bromophenyl)-3,3-difluorocyclobutyl)methyl)-4-methyl-4H-1,2,4-triazole (Intermediate P; 65 mg, 0.19 mmol), Me₄tButylXphos (9.1 mg, 0.02 mmol), tris(dibenzylideneacetone)dipalladium(0) (7.0 mg, 0.01 mmol), K₃PO₄ (80.6 mg, 0.38 mmol) and 4 Å molecular sieve (note that base and MS were flame dried prior to addition). The vial was purged with nitrogen before degassed tert-butanol (2.5 mL) was added and the vial was sealed. The reaction mixture was stirred at 110° C. for 15 h. The reaction was purified by chromatography on silica gel (2-15% MeOH in DCM) to afford 2-(3-(3,3-difluoro-1-((4-methyl-4H-1,2,4-triazol-3-yl)methyl)cyclobutyl)-phenyl)-4-(difluoromethyl)-6-(hydroxymethyl)isoindolin-1-one (61.7 mg, 68% yield). LCMS (ESI) m/z: 475.1 [M+H]⁺.

A second intermediate, 2-(3-(3,3-difluoro-1-((4-methyl-4H-1,2,4-triazol-3-yl)-methyl)cyclobutyl)phenyl)-7-(difluoromethyl)-3-oxoisoindoline-5-carbaldehyde, can be synthesized as follows. Dess-Martin periodinane (107.3 mg, 0.25 mmol) was added to a suspension of 2-(3-(3,3-difluoro-1-((4-methyl-4H-1,2,4-triazol-3-yl)methyl)cyclobutyl)phenyl)-4-(difluoromethyl)-6-(hydroxymethyl)isoindolin-1-one (60 mg, 0.13 mmol) in DCM (0.84 mL) at 0° C. The resulting mixture was allowed to warm to rt and stirred for the weekend. The reaction was quenched with saturated aqueous Na₂S₂O₃ solution and saturated aqueous NaHCO₃ solution and the resulting mixture was stirred vigorously for 30 minutes. The layers were separated and the aqueous phase was extracted with 4:1 CHCl₃/IPA (3×). The combined organic phases were dried over magnesium sulfate, filtered and concentrated to afford 2-(3-(3,3-difluoro-1-((4-methyl-4H-1,2,4-triazol-3-yl)methyl)cyclobutyl)phenyl)-7-(difluoromethyl)-3-oxoisoindoline-5-carbaldehyde. (57.6 mg, 96% yield) as crude used as such for in the next step. LCMS (ESI) m/z: 473.1 [M+H]⁺.

To form Compound 250, a microwave vial was charged with 2-(3-(3,3-difluoro-1-((4-methyl-4H-1,2,4-triazol-3-yl)methyl)cyclobutyl)phenyl)-7-(difluoromethyl)-3-oxoisoindoline-5-carbaldehyde (50 mg, 0.11 mmol), triethylamine (88.5 μL, 0.64 mmol) and 1-methylcyclobutanamine hydrochloride (64.4 mg, 0.53 mmol) in methanol (0.88 mL). The reaction was heated in a microwave oven for 3 minutes at 100° C. The reaction was cooled to rt and sodium cyanoborohydride (20.0 mg, 0.32 mmol) was added. The reaction was heated in a microwave oven at 100° C. for 60 minutes. The reaction was diluted with saturated aqueous NaHCO₃ solution, extracted with EtOAc, dried over sodium sulfate, filtered and evaporated. The organic layers were combined, dried over sodium sulfate, and concentrated. The crude residue was purified by chromatography on C18 silica gel (20-60% acetonitrile in 0.5% formic acid buffer). Appropriate fractions were concentrated, frozen and lyophilized to afford 2-(3-(3,3-difluoro-1-((4-methyl-4H-1,2,4-triazol-3-yl)methyl)cyclobutyl)-phenyl)-4-(difluoromethyl)-6-(((1-methylcyclobutyl)amino)methyl)isoindolin-1-one (26.8 mg, 47% yield). LCMS (ESI) m/z: 542.2 [M+H]⁺. ¹H NMR (400 MHz, DMSO-d6) δ 8.22 (s, 1H), 8.17 (s, 1H), 7.96-7.83 (m, 3H), 7.41 (s, 1H), 7.34 (t, J=8.0 Hz, 1H), 7.41-7.12 (m, 1H), 6.76 (d, J=7.8 Hz, 1H), 5.05 (s, 2H), 3.80 (s, 2H), 3.33-3.28 (m, 2H), 3.26-3.23 (m, 2H), 3.02 (q, J=13.8 Hz, 2H), 2.73 (s, 3H), 2.04-1.94 (m, 2H), 1.77-1.63 (m, 4H), 1.24 (s, 3H).

### Example 61: Compound 251

Compound 251 (2-(4-(1-((4-methyl-4H-1,2,4-triazol-3-yl)methyl)cyclobutyl)-6-(methylthio)pyridin-2-yl)-6-(((1-methylcyclobutyl)amino)methyl)-4-(trifluoromethyl)isoindolin-1-one) can be synthesized according to Scheme 59, FIG. 41.

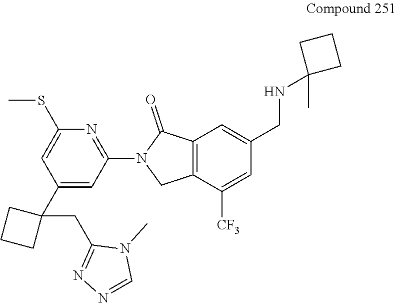 [SMILES: CSc1cc(C2(Cc3nncn3C)CCC2)cc(N2Cc3c(cc(CNC4(C)CCC4)cc3C(F)(F)F)C2=O)n1]

A first intermediate, 2-chloro-4-(1-((4-methyl-4H-1,2,4-triazol-3-yl)methyl)cyclo-butyl)-6-(methylthio)pyridine, can be synthesized as follows. To a solution of 2,6-dichloro-4-(1-((4-methyl-4H-1,2,4-triazol-3-yl)methyl)cyclobutyl)pyridinee (Intermediate U; 30 mg, 0.10 mmol) in 1,4-dioxane (0.5 mL) was added sodium thiomethoxyde (53 mg, 0.75 mmol) and the reaction was heated at 60° C. for 16 h. The reaction was diluted with saturated aqueous NaHCO₃ solution, extracted with EtOAc, dried over sodium sulfate, filtered and evaporated to afford 2-chloro-4-(1-((4-methyl-4H-1,2,4-triazol-3-yl)methyl)cyclobutyl)-6-(methylthio)-pyridine (29.8 mg, 96% yield). Crude residue was used like that in the next step. LCMS (ESI) m/z: 309.2, 311.0 [M+H]⁺.

A second intermediate, tert-butyl ((2-(4-(1-((4-methyl-4H-1,2,4-triazol-3-yl)methyl)cyclobutyl)-6-(methylthio)pyridin-2-yl)-3-oxo-7-(trifluoromethyl)isoindolin-5-yl)methyl)(1-methylcyclobutyl)carbamate, can be synthesized as follows. A vial was charged with K₃PO₄ (38.5 mg, 0.18 mmol) and 4 Å molecular sieve then flame dry. tert-butyl (1-methylcyclobutyl)((3-oxo-7-(trifluoromethyl)isoindolin-5-yl)methyl)carbamate (Intermediate R; 54.2 mg, 0.14 mmol), 2-chloro-4-(1-((4-methyl-4H-1,2,4-triazol-3-yl)methyl)-cyclobutyl)-6-(methylthio)pyridine (28 mg, 0.09 mmol), Me₄tButylXphos (8.7 mg, 0.02 mmol), tris(dibenzylideneacetone)dipalladium(0) (6.6 mg, 0.01 mmol) were added. The vial was purged with nitrogen before degassed tert-butanol (0.45 mL) was added and the vial was sealed. The reaction mixture was stirred at 110° C. for 15 h. The crude product was purified by chromatography on silica gel (2-15% of MeOH in DCM) to afford tert-butyl ((2-(4-(1-((4-methyl-4H-1,2,4-triazol-3-yl)methyl)cyclobutyl)-6-(methylthio)pyridin-2-yl)-3-oxo-7-(trifluoromethyl)isoindolin-5-yl)methyl)(1-methylcyclobutyl)carbamate (24 mg, 39% yield) LCMS (ESI) m/z: 671.3 [M+H]⁺.

To make Compound 251, to a solution of tert-butyl ((2-(4-(1-((4-methyl-4H-1,2,4-triazol-3-yl)methyl)cyclobutyl)-6-(methylthio)pyridin-2-yl)-3-oxo-7-(trifluoromethyl)isoindolin-5-yl)methyl)(1-methylcyclobutyl)carbamate (24 mg, 0.04 mmol) in DCM (0.18 mL) was added trifluoroacetic acid (150 μL). The reaction was stirred at rt for 2 h. The reaction was diluted with saturated aqueous NaHCO₃ solution, extracted with EtOAc, dried over sodium sulfate, filtered and evaporated. The crude product was purified by semi-prep. LCMS (50-70% gradient of acetonitrile in ammonium bicarbonate buffer, pH=10). Appropriate fractions were concentrated, frozen and lyophilized to provide 2-(4-(1-((4-methyl-4H-1,2,4-triazol-3-yl)methyl)cyclobutyl)-6-(methylthio)pyridin-2-yl)-6-(((1-methylcyclobutyl)amino)-methyl)-4-(trifluoromethyl)isoindolin-1-one (19.5 mg, 96% yield). LCMS (ESI) m/z: 571.1 [M+H]⁺. ¹H NMR (400 MHz, DMSO-d6) δ 8.20 (s, 1H), 8.04 (d, J=11.2 Hz, 2H), 7.95 (d, J=1.1 Hz, 1H), 6.63 (d, J=1.1 Hz, 1H), 5.22 (s, 2H), 3.82 (d, J=7.4 Hz, 2H), 3.24 (s, 2H), 3.07 (s, 3H), 2.50 (s, 3H), 2.39-2.28 (m, 2H), 2.18-2.07 (m, 1H), 2.04-1.93 (m, 2H), 1.90-1.78 (m, 1H), 1.76-1.62 (m, 4H), 1.23 (s, 3H).

### Example 62: Compound 252

Compound 252 (4-chloro-2-(3-(3,3-difluoro-1-((4-methyl-4H-1,2,4-triazol-3-yl)-methyl)cyclobutyl)phenyl)-6-(((1-methylcyclobutyl)amino)methyl)isoindolin-1-one formate) can be synthesized as follows.

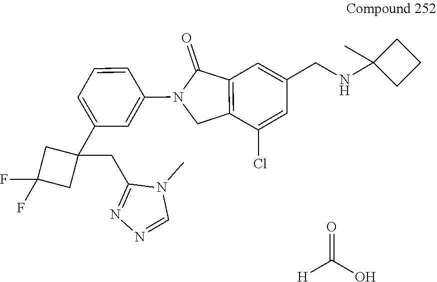 [SMILES: Cn1cnnc1CC1(c2cccc(N3Cc4c(Cl)cc(CNC5(C)CCC5)cc4C3=O)c2)CC(F)(F)C1 ; O=CO]

To a stirred solution of 7-chloro-2-(3-(3,3-difluoro-1-((4-methyl-4H-1,2,4-triazol-3-yl)methyl)cyclobutyl)phenyl)-3-oxoisoindoline-5-carbaldehyde (prepared following the same procedures shown for compound 250, Scheme 58, FIG. 40; 45 mg, 0.10 mmol) and 1-methylcyclobutanamine hydrochloride (35.9 mg, 0.30 mmol) in DCE (1.1 mL) was added NaBH(OAc)₃ (208.8 mg, 0.98 mmol). The suspension was stirred at rt for 20 h. The volatiles were evaporated and the residue was purified by semi-prep. LCMS (15-35% gradient of acetonitrile in ammonium formate buffer, pH=3.8). Appropriate fractions were concentrated, frozen and lyophilized to provide 4-chloro-2-(3-(3,3-difluoro-1-((4-methyl-4H-1,2,4-triazol-3-yl)methyl)cyclobutyl)phenyl)-6-(((1-methylcyclobutyl)amino)methyl)isoindolin-1-one formate (11 mg, 21% yield). LCMS (ESI) m/z: 526.31 [M+H]⁺. ¹H NMR (400 MHz, DMSO-d6) δ 8.39 (s, 1H), 8.18 (s, 1H), 7.95 (d, J=7.7 Hz, 1H), 7.75 (s, 2H), 7.40-7.29 (m, 2H), 6.77 (d, J=7.2 Hz, 1H), 4.92 (s, 2H), 3.75 (s, 3H), 3.33-3.20 (m, 4H), 3.02 (dd, J=27.7, 13.9 Hz, 2H), 2.73 (s, 3H), 2.03-1.92 (m, 2H), 1.75-1.62 (m, 4H), 1.23 (s, 3H).

### Example 63: Compound 253

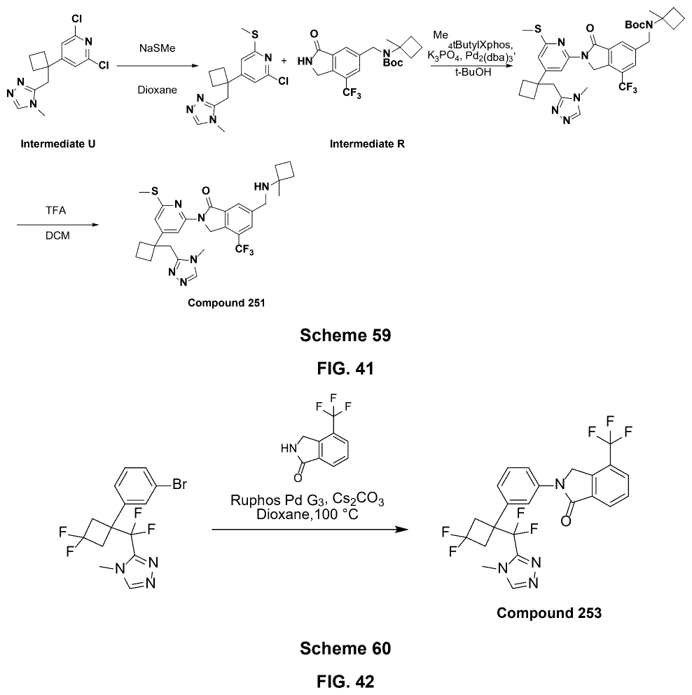

Compound 253 (2-(3-(1-(difluoro(4-methyl-4H-1,2,4-triazol-3-yl)methyl)-3,3-difluorocyclobutyl)phenyl)-4-(trifluoromethyl)isoindolin-1-one) can be synthesized according to Scheme 60, FIG. 42.

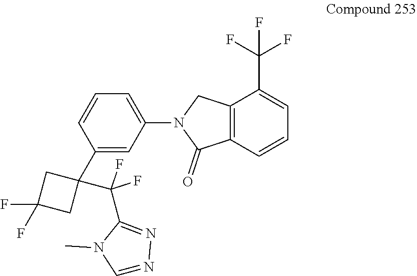 [SMILES: Cn1cnnc1C(F)(F)C1(c2cccc(N3Cc4c(cccc4C(F)(F)F)C3=O)c2)CC(F)(F)C1]

To a solution of 3-((1-(3-bromophenyl)-3,3-difluorocyclobutyl)difluoromethyl)-4-methyl-4H-1,2,4-triazole (made following the procedure shown for Intermediate E; 90.0 mg, 0.24 mmol), 4-(trifluoromethyl)isoindolin-1-one (52.7 mg, 0.26 mmol), cesium carbonate (232.6 mg, 2.71 mmol) in 1,4-dioxane (4 mL) was added (2-dicyclohexylphosphino-2′,6′-diisopropoxy-1,1′-biphenyl)[2-(2′-amino-1,1′biphenyl)]palladium(II) methanesulfonate (39.8 mg, 0.05 mmol, CAS No.: 1445085-77-7). The reaction mixture was stirred at 100° C. for 16 h under nitrogen protection and concentrated under reduced pressure. The residue was purified by RP-HPLC (42% to 72% ACN/0.2% formic acid in water)) to afford 2-(3-(1-(difluoro(4-methyl-4H-1,2,4-triazol-3-yl)methyl)-3,3-difluorocyclobutyl)phenyl)-4-(trifluoromethyl)isoindolin-1-one (18 mg, 15% yield). LCMS [M+H]⁺=499.0.

¹H NMR (400 MHz, methanol-d₄) δ 8.33 (s, 1H), 8.10 (d, J=7.6 Hz, 1H), 7.98 (d, J=7.6 Hz, 1H), 7.85-7.74 (m, 2H), 7.67 (s, 1H), 7.47 (t, J=8.0 Hz, 1H), 7.06 (d, J=8.0 Hz, 1H), 5.12 (s, 2H), 3.79-3.66 (m, 2H), 3.28-3.18 (m, 2H), 2.97 (s, 3H).

### Example 64: Compound 254 and Compound 255

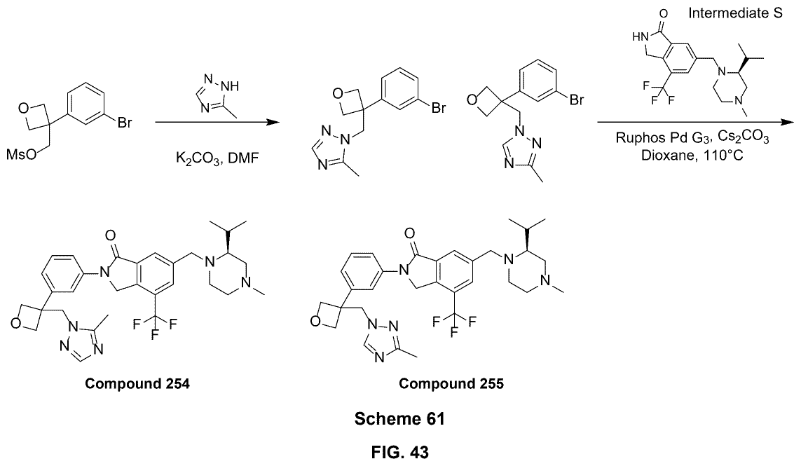

Compounds 254 ((S)-6-((2-isopropyl-4-methylpiperazin-1-yl)methyl)-2-(3-(3-((5-methyl-1H-1,2,4-triazol-1-yl)methyl)oxetan-3-yl)phenyl)-4-(trifluoromethyl)isoindolin-1-one) and 255 ((S)-6-((2-isopropyl-4-methylpiperazin-1-yl)methyl)-2-(3-(3-((3-methyl-1H-1,2,4-triazol-1-yl)methyl)oxetan-3-yl)phenyl)-4-(trifluoromethyl)isoindolin-1-one) can be synthesized according to Scheme 61, FIG. 43.

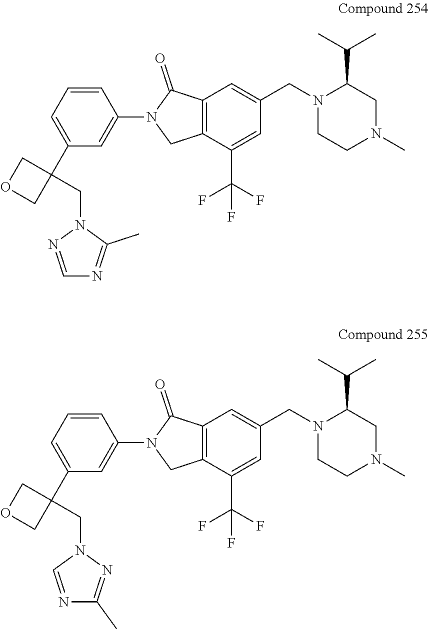 [SMILES: Cc1ncnn1CC1(c2cccc(N3Cc4c(cc(CN5CCN(C)C[C@@H]5C(C)C)cc4C(F)(F)F)C3=O)c2)COC1 ; Cc1ncn(CC2(c3cccc(N4Cc5c(cc(CN6CCN(C)C[C@@H]6C(C)C)cc5C(F)(F)F)C4=O)c3)COC2)n1]

An isomeric pair of first intermediates, 1-((3-(3-bromophenyl)oxetan-3-yl)methyl)-5-methyl-1H-1,2,4-triazole and 1-((3-(3-bromophenyl)oxetan-3-yl)methyl)-3-methyl-1H-1,2,4-triazole, can be synthesized as follows. A solution of (3-(3-bromophenyl)oxetan-3-yl)methyl methanesulfonate (1.5 g, 4.67 mmol), potassium carbonate (1.29 g, 9.34 mmol) and 5-methyl-1H-1,2,4-triazole (426.9 mg, 5.14 mmol) in N,N-Dimethylformamide (40.0 mL) was stirred at 100° C. for 2 h. After cooled, the mixture was filtered then concentrated. The residue was purified by RP-HPLC (21% to 51% ACN/(0.225% FA in water)) to afford the mixture of 1-((3-(3-bromophenyl)oxetan-3-yl)methyl)-5-methyl-1H-1,2,4-triazole and 1-((3-(3-bromophenyl)oxetan-3-yl)methyl)-3-methyl-1H-1,2,4-triazole (800 mg, 56% yield) as a colorless oil. LCMS: [M+H]⁺=309.5.

In a glove box, a mixture of the 1-((3-(3-bromophenyl)oxetan-3-yl)methyl)-5-methyl-1H-1,2,4-triazole and 1-((3-(3-bromophenyl)oxetan-3-yl)methyl)-3-methyl-1H-1,2,4-triazole (300.0 mg, 0.97 mmol), with (S)-6-((2-isopropyl-4-methylpiperazin-1-yl)methyl)-4-(trifluoromethyl)isoindolin-1-one (Intermediate S; 380.6 mg, 1.07 mmol), cesium carbonate (951.6 mg, 2.92 mmol) and (2-dicyclohexylphosphino-2′,6′-diisopropoxy-1,1′-biphenyl)[2-(2′-amino-1, 1′biphenyl)]palladium(II) methanesulfonate (81.4 mg, 0.10 mmol, CAS #: 1445085-77-7) in 1,4-dioxane (10 mL) was stirred at 100° C. for 2 h and concentrated under reduced pressure. The residue was purified by RP-HPLC (15 to 45% ACN/(0.225% FA in water)) to afford the mixture of regio-isomers (320 mg, 56% yield) as colorless oil. LCMS: [M+H]⁺=583.3.

The above mixture was further purified by chiral SFC (Column=G_3_EtOH_DEA_40_28ML; Column dimensions=250 mm×30 mm×10 μm; Detection wavelength=220 nM; Flow rate=80 mL/min; Run time=7 min; Column temperature=25° C.) with 0.1 NH₃·H₂O-50% ethanol-carbon dioxide) to give a first product, Compound 255: (S)-6-((2-isopropyl-4-methylpiperazin-1-yl)methyl)-2-(3-(3-((3-methyl-1H-1,2,4-triazol-1-yl)methyl)oxetan-3-yl)phenyl)-4-(trifluoromethyl)isoindolin-1-one (Peak 1, retention time=1.890 min) (93.7 mg, 31% yield) as a white solid. ¹H NMR (400 MHz, methanol-d₄) δ 8.09 (s, 1H), 7.95 (s, 1H), 7.79-7.77 (m, 1H), 7.68 (s, 1H), 7.42-7.38 (m, 2H), 6.76 (d, J=8.0 Hz, 1H), 5.11-5.05 (m, 6H), 4.78 (s, 2H), 4.33 (d, J=6.4 Hz, 1H), 3.34 (d, J=6.4 Hz, 1H), 2.85-2.73 (m, 3H), 2.38-2.13 (m, 11H), 1.03-0.98 (m, 6H). LCMS: [M+H]+=583.2.

The mixture of other fractions (Peak 2 and Peak 3, retention time=2.553 min and 3.641 min) was further purified by chiral SFC (Column=CAS-SH-ANA-SFC-G; Column dimensions=250 mm×30 mm×10 μm; Detection wavelength=220 nM; Flow rate=2.5 mL/min; Run time=6 min; Column temperature=25° C.) with 0.1 NH₃·H₂O-30% ethanol-carbon dioxide) to give Compound 254: (S)-6-((2-isopropyl-4-methylpiperazin-1-yl)methyl)-2-(3-(3-((5-methyl-1H-1,2,4-triazol-1-yl)methyl)oxetan-3-yl)phenyl)-4-(trifluoromethyl)-isoindolin-1-one (Peak 1, retention time=3.876 min) (40.5 mg, 41% yield). ¹H NMR (400 MHz, methanol-d₄) δ 8.08 (s, 1H), 7.94 (s, 1H), 7.83-7.81 (m, 1H), 7.41-7.37 (m, 1H), 7.21 (s, 1H), 6.70 (d, J=8.0 Hz, 1H), 5.20-5.02 (m, 6H), 4.79 (s, 2H), 4.33 (d, J=16.4 Hz, 1H), 3.34-3.31 (m, 1H), 2.82-2.71 (m, 3H), 2.34-2.09 (m, 8H), 1.63 (s, 3H), 1.03-0.97 (m, 6H). LCMS: [M+H]⁺=583.3.

### Example 65: Compound 256, Compound 257, and Compound 258

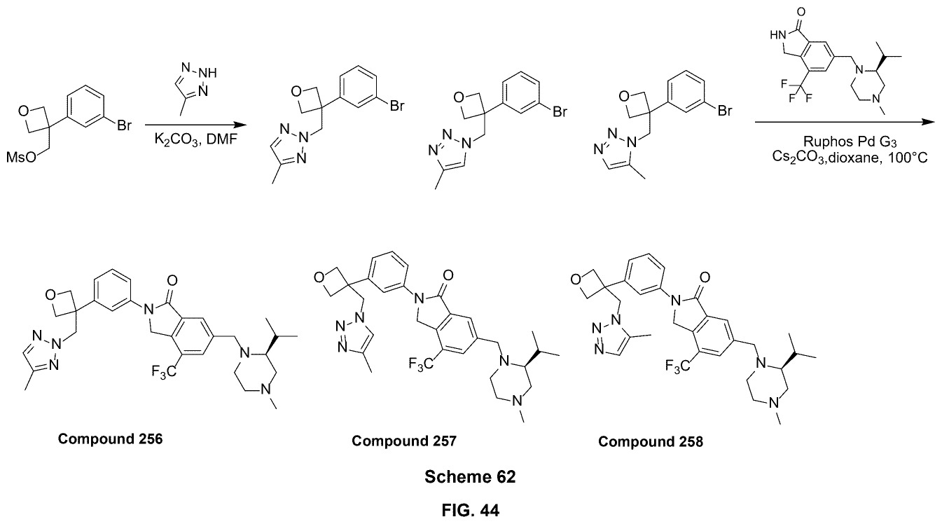

Compounds 256 ((S)-6-((2-isopropyl-4-methylpiperazin-1-yl)methyl)-2-(3-(3-((4-methyl-2H-1,2,3-triazol-2-yl)methyl)oxetan-3-yl)phenyl)-4-(trifluoromethyl)isoindolin-1-one), 257 ((S)-6-((2-isopropyl-4-methylpiperazin-1-yl)methyl)-2-(3-(3-((4-methyl-1H-1,2,3-triazol-1-yl)methyl)oxetan-3-yl)phenyl)-4-(trifluoromethyl)isoindolin-1-one) and 258 ((S)-6-((2-isopropyl-4-methylpiperazin-1-yl)methyl)-2-(3-(3-((5-methyl-1H-1,2,3-triazol-1-yl)methyl)-oxetan-3-yl)phenyl)-4-(trifluoromethyl)isoindolin-1-one) can be synthesized according to Scheme 62, FIG. 44

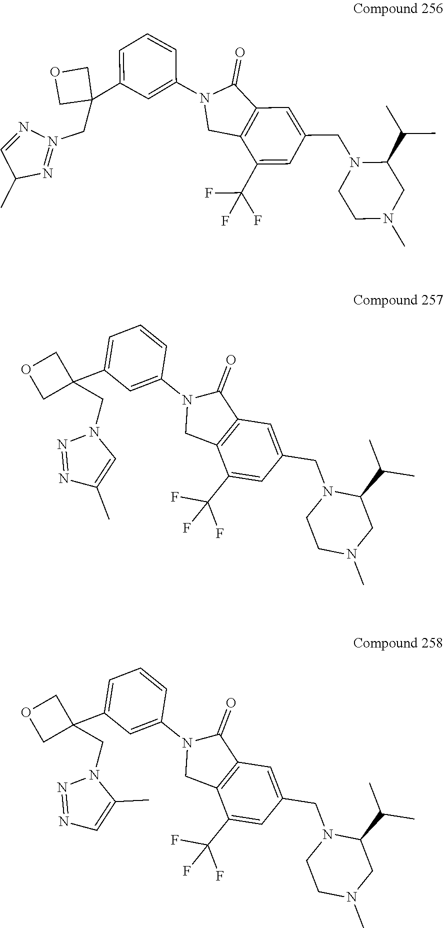

A set of first (isomeric) intermediates, 2-((3-(3-bromophenyl)oxetan-3-yl)methyl)-4-methyl-2H-1,2,3-triazole, 1-((3-(3-bromophenyl)oxetan-3-yl)methyl)-4-methyl-1H-1,2,3-triazole, and 1-((3-(3-bromophenyl)oxetan-3-yl)methyl)-5-methyl-1H-1,2,3-triazole, can be synthesized as follows. To a solution of (3-(3-bromophenyl)oxetan-3-yl)methyl methanesulfonate (1.5 g, 4.67 mmol) in N,N-dimethylformamide (15 mL) was added 4-methyl-2H-1,2,3-triazole (427 mg, 5.14 mmol) and potassium carbonate (1.3 g, 9.34 mmol) at 25° C. The mixture was stirred at 100° C. for 2 hand filtered. The filtrate was concentrated under reduced pressure and the residue was purified by RP-HPLC (40% to 70% ACN/0.2% formic acid in water)) to afford the mixture of 1-((3-(3-bromophenyl)oxetan-3-yl)methyl)-4-methyl-1H-1,2,3-triazole and 1-((3-(3-bromophenyl)oxetan-3-yl)methyl)-5-methyl-1H-1,2,3-triazole (240 mg, 17% yield) as a yellow oil, and 2-((3-(3-bromophenyl)-oxetan-3-yl)methyl)-4-methyl-2H-1,2,3-triazole (600 mg, 42% yield) as a yellow oil. ¹H NMR (400 MHz, DMSO-d6) δ 7.44-7.36 (m, 2H), 7.23-7.14 (m, 2H), 6.93 (d, J=7.6 Hz, 1H), 4.97 (d, J=6.4 Hz, 2H), 4.92 (s, 2H), 4.82 (d, J=6.4 Hz, 2H), 2.14 (s, 3H).

Each of these intermediates can be reacted to form each of the product compounds, respectively.

In a glove box, a solution of 2-((3-(3-bromophenyl)oxetan-3-yl)methyl)-4-methyl-2H-1,2,3-triazole (70.0 mg, 0.23 mmol), (S)-6-((2-isopropyl-4-methylpiperazin-1-yl)methyl)-4-(trifluoromethyl)isoindolin-1-one (Intermediate S; 88.8 mg, 0.25 mmol), cesium carbonate (222.0 mg, 0.68 mmol) and (2-dicyclohexylphosphino-2′,6′-diisopropoxy-1,1′-biphenyl)[2-(2′-amino-1,1′biphenyl)]palladium(II) methanesulfonate (19.0 mg, 0.02 mmol, CAS No.: 1445085-77-7) in 1,4-dioxane (4 mL) was stirred at 100° C. for 2 h. After cooling, the mixture was filtered and the filtrate was concentrated under reduced pressure. The residue was purified by RP-HPLC (55 to 85% ACN/(0.05% NH₃·H₂O+10 mM NH₄HCO₃ in water)) to afford Compound 256 (S)-6-((2-isopropyl-4-methylpiperazin-1-yl)methyl)-2-(3-(3-((4-methyl-2H-1,2,3-triazol-2-yl)methyl)oxetan-3-yl)phenyl)-4-(trifluoromethyl)isoindolin-1-one (100 mg, 42% yield) as a white solid. ¹H NMR (400 MHz, methanol-d₄) δ 8.08 (s, 1H), 7.94 (s, 1H), 7.77 (d, J=8.4 Hz, 1H), 7.42-7.28 (m, 3H), 6.80 (d, J=8.0 Hz, 1H), 5.16 (d, J=6.4 Hz, 2H), 5.08-5.04 (m, 4H), 4.96 (s, 2H), 4.34 (d, J=14.0 Hz, 1H), 3.34 (s, 1H), 2.82-2.65 (m, 3H), 2.38-2.27 (m, 6H), 2.20 (s, 3H), 2.19-2.03 (m, 2H), 1.02-0.97 (m, 6H). LCMS [M+H]⁺=583.4.

In a glove box, a solution of 1-((3-(3-bromophenyl)oxetan-3-yl)methyl)-4-methyl-1H-1,2,3-triazole and 1-((3-(3-bromophenyl)oxetan-3-yl)methyl)-5-methyl-1H-1,2,3-triazole (240 mg, 0.78 mmol), (S)-6-((2-isopropyl-4-methylpiperazin-1-yl)methyl)-4-(trifluoromethyl)isoindolin-1-one (Intermediate S; 305 mg, 0.86 mmol), cesium carbonate (761 mg, 2.34 mmol), (2-dicyclohexylphosphino-2′,6′-diisopropoxy-1,1′-biphenyl)[2-(2′-amino-1,1′biphenyl)]palladium(II) methanesulfonate (65 mg, 0.08 mmol, CAS No.: 1445085-77-7) in 1,4-Dioxane (10 mL) at 100 C for 16 h and concentrated under reduced pressure. The residue was first purified by RP-HPLC (50% to 80% ACN/(0.05% NH₃·H₂O+10 mM NH₄HCO₃ in water)), then by chiral SFC (Column=daicel chiralpak ig; Column dimensions=250 mm×30 mm×10 μm; Detection wavelength=220 nM; Flow rate=80 mL/min; Run time=4.0 min; Column temperature=25° C.) with 0.1% ammonium hydroxide-60% ethanol-carbon dioxide) to afford two compounds, characterized as follows.

Compound 257: (S)-6-((2-isopropyl-4-methylpiperazin-1-yl)methyl)-2-(3-(3-((4-methyl-1H-1,2,3-triazol-1-yl)methyl)oxetan-3-yl)phenyl)-4-(trifluoromethyl)isoindolin-1-one (Peak 1, retention time=0.858 min) (23 mg, 44% yield). ¹H NMR (400 MHz, methanol-d₄) δ 8.09 (s, 1H), 7.95 (s, 1H), 7.80-7.79 (m, 1H), 7.44-7.33 (m, 2H), 7.22 (s, 1H), 6.77 (d, J=7.2 Hz, 1H), 5.08 (s, 2H), 5.06 (s, 4H), 5.01 (s, 2H), 4.34 (d, J=14.4 Hz, 1H), 3.35 (s, 1H), 2.83-2.66 (m, 3H), 2.40-2.33 (m, 2H), 2.30 (s, 4H), 2.18 (s, 4H), 2.14-2.05 (m, 1H), 1.03-0.98 (m, 6H.).

Compound 258: (S)-6-((2-isopropyl-4-methylpiperazin-1-yl)methyl)-2-(3-(3-((5-methyl-1H-1,2,3-triazol-1-yl)methyl)oxetan-3-yl)phenyl)-4-(trifluoromethyl)isoindolin-1-one (Peak 2, retention time=1.766 min) (5.5 mg, 11% yield). ¹H NMR (400 MHz, methanol-d₄) δ 8.08 (s, 1H), 7.94 (s, 1H), 7.82-7.80 (m, 1H), 7.36 (t, J=7.6 Hz, 1H), 7.27-7.23 (m, 2H), 6.63 (d, J=7.6 Hz, 1H), 5.20 (d, J=6.4 Hz, 2H), 5.08 (d, J=6.4 Hz, 2H), 5.02 (s, 2H), 4.97 (s, 2H), 4.34 (d, J=14.0 Hz, 1H), 3.34 (s, 1H), 2.84-2.63 (m, 3H), 2.41-2.25 (m, 6H), 2.21-2.04 (m, 2H), 1.55 (s, 3H), 1.02-0.97 (m, 6H).

### Example 66: Compound 259 and Compound 260

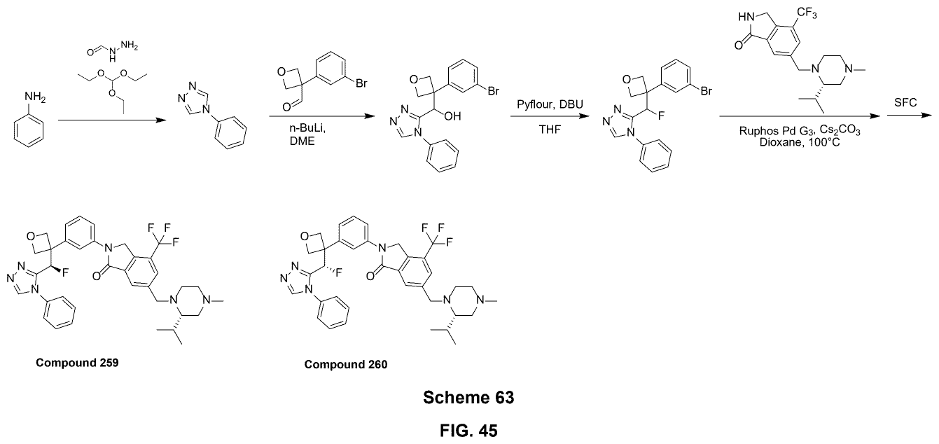

Compounds 259 (2-(3-(3-((R)-fluoro(4-phenyl-4H-1,2,4-triazol-3-yl)methyl)oxetan-3-yl)phenyl)-6-(((S)-2-isopropyl-4-methylpiperazin-1-yl)methyl)-4-(trifluoromethyl)isoindolin-1-one) and 260 (2-(3-(3-((S)-fluoro(4-phenyl-4H-1,2,4-triazol-3-yl)methyl)oxetan-3-yl)-phenyl)-6-(((S)-2-isopropyl-4-methylpiperazin-1-yl)methyl)-4-(trifluoromethyl)isoindolin-1-one) can be synthesized according to Scheme 63, FIG. 45.

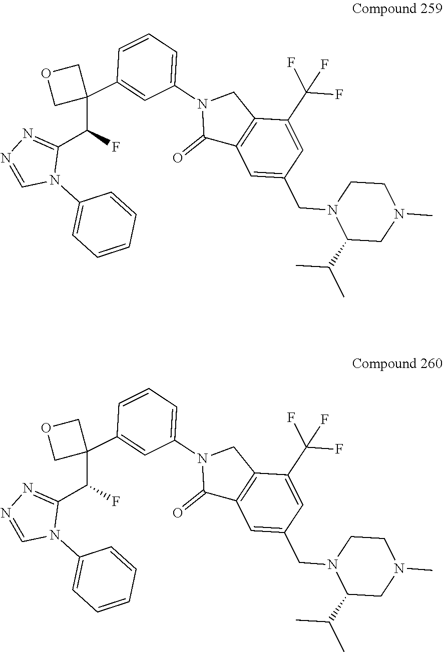 [SMILES: CC(C)[C@H]1CN(C)CCN1Cc1cc2c(c(C(F)(F)F)c1)CN(c1cccc(C3([C@@H](F)c4nncn4-c4ccccc4)COC3)c1)C2=O ; CC(C)[C@H]1CN(C)CCN1Cc1cc2c(c(C(F)(F)F)c1)CN(c1cccc(C3([C@H](F)c4nncn4-c4ccccc4)COC3)c1)C2=O]

A first intermediate, 4-phenyl-4H-1,2,4-triazole, can be synthesized as follows. To a solution of formohydrazide (10.0 g, 166.5 mmol) in methanol (100 mL) was added triethoxymethane (33.2 mL, 199.8 mmol). The mixture was stirred at 80° C. for 2 h, then aniline (18.2 mL, 199.8 mmol) was added. The resulting mixture was stirred at 80° C. for another 16 h and concentrated under reduced pressure. The residue was purified by silica gel chromatography (mobile phase: methanol/dichloromethane, gradient 0% to 10%) to afford 4-phenyl-4H-1,2,4-triazole (22 g, 91% yield) as a white solid. ¹H NMR (400 MHz, DMSO-d₆) δ 9.17-9.11 (m, 2H), 7.73-7.69 (m, 2H), 7.59-7.54 (m, 2H), 7.48-7.42 (m, 1H).

A second intermediate, (3-(3-bromophenyl)oxetan-3-yl)(4-phenyl-4H-1,2,4-triazol-3-yl)methanol, can be synthesized as follows. To a solution of 4-phenyl-4H-1,2,4-triazole (3.61 g, 24.89 mmol) in 1,2-dimethoxyethane (150 mL) was added n-butyllithium (2.5 M in hexane, 9.9 mL, 24.8 mmol) at −50° C. over 5 minutes. The resulting mixture was stirred for 1 h at −50° C. and then a solution of 3-(3-bromophenyl)oxetane-3-carbaldehyde (4.00 g, 16.6 mmol) in 1,2-dimethoxyethane (10 mL) was added dropwise. The reaction mixture was allowed to warm to 0° C. over 1 h then quenched with water (30 mL). The resulting solution was extracted with dichloromethane (3×100 mL). The combined organic layers were dried over anhydrous sodium sulfate and concentrated under reduced pressure. The residue was purified by silica gel chromatography (mobile phase: methanol/dichloromethane, gradient 0% to 10%) to afford (3-(3-bromophenyl)oxetan-3-yl)(4-phenyl-4H-1,2,4-triazol-3-yl)-methanol (2.50 g, 39% yield) as a yellow solid. LCMS [M+H]⁺=386.0&388.0.

A third intermediate, 3-((3-(3-bromophenyl)oxetan-3-yl)fluoromethyl)-4-phenyl-4H-1,2,4-triazole, can be synthesized as follows. To a solution of (3-(3-bromophenyl)oxetan-3-yl)(4-phenyl-4H-1,2,4-triazol-3-yl)methanol (2.5 g, 6.5 mmol) in dichloromethane (120 mL) was added N,N-diethylaminosulfur trifluoride (DAST) (5.1 mL, 38.9 mmol) at 0° C. The mixture was stirred for 16 h at 0° C. and quenched by addition of saturated aqueous sodium bicarbonate (10 mL). The resulting mixture was extracted with dichloromethane (3×100 mL). The combined organic layers were dried over anhydrous sodium sulfate and concentrated under reduced pressure. The residue was purified by silica gel chromatography (mobile phase: methanol/dichloromethane, gradient 0%-4%) to afford 3-((3-(3-bromophenyl)oxetan-3-yl)fluoromethyl)-4-phenyl-4H-1,2,4-triazole (1.7 g, 68% yield) as a yellow oil. LCMS [M+H]⁺=388.0 and 390.0.

In a glove box, a mixture of 3-((3-(3-bromophenyl)oxetan-3-yl)fluoromethyl)-4-phenyl-4H-1,2,4-triazole (50.0 mg, 0.13 mmol), (S)-6-((2-isopropyl-4-methylpiperazin-1-yl)-methyl)-4-(trifluoromethyl)isoindolin-1-one (Intermediate S; 50.4 mg, 0.14 mmol), cesium carbonate (126 mg, 0.39 mmol) and (2-dicyclohexylphosphino-2′,6′-diisopropoxy-1,1′-biphenyl)[2-(2′-amino-1,1′biphenyl)]palladium(II) methanesulfonate (10.8 mg, 0.01 mmol, CAS #1445085-77-7) in 1,4-dioxane (2 mL) was stirred at 100° C. for 16 h and concentrated under reduce pressure. The residue was purified by preparative TLC (solvent gradient: 10% methanol in dichloromethane) to afford a racemate of 2-(3-(3-(fluoro(4-phenyl-4H-1,2,4-triazol-3-yl)methyl)oxetan-3-yl)phenyl)-6-(((S)-2-isopropyl-4-methylpiperazin-1-yl)methyl)-4-(trifluoromethyl)isoindolin-1-one (50 mg, 59% yield) as a yellow oil.

The above mixture was further purified by chiral SFC (Column=Daicel Chiralpak IG; Column dimensions=250 mm×30 mm×10 μm; Detection wavelength=220 nM; Flow rate=80 mL/min; Mobile phase: A: CO₂ B: ethanol (0.05% DEA); Isocratic: 40% B) with 0.1% ammonium hydroxide) to afford two isomers, characterized as follows.

2-(3-(3-((R)-fluoro(4-phenyl-4H-1,2,4-triazol-3-yl)methyl)oxetan-3-yl)phenyl)-6-(((S)-2-isopropyl-4-methylpiperazin-1-yl)methyl)-4-(trifluoromethyl)isoindolin-1-one (Peak 1, retention time=1.085 min) (23 mg, 46% yield). ¹H NMR (400 MHz, methanol-d₄) δ 8.52 (s, 1H), 8.09 (s, 1H), 7.95 (s, 1H), 7.87 (d, J=7.6 Hz, 1H), 7.40-7.33 (m, 5H), 6.90-6.86 (m, 2H), 6.82 (d, J=8.0 Hz, 1H), 5.73 (d, J=46.0 Hz, 1H), 5.72 (d, J=6.4 Hz, 1H), 5.32-5.27 (m, 1H), 5.26-5.21 (m, 1H), 5.04-4.92 (m, 2H), 4.92-4.90 (m, 1H), 4.34 (d, J=14.4 Hz, 1H), 3.34 (d, J=14.0 Hz, 1H), 2.85 (d, J=10.8 Hz, 1H), 2.79-2.68 (m, 2H), 2.42-2.09 (m, 8H), 1.01 (d, J=6.4 Hz, 3H), 0.98 (d, J=6.4 Hz, 3H). LCMS: [M+H]⁺=663.2.

2-(3-(3-((S)-fluoro(4-phenyl-4H-1,2,4-triazol-3-yl)methyl)oxetan-3-yl)phenyl)-6-(((S)-2-isopropyl-4-methylpiperazin-1-yl)methyl)-4-(trifluoromethyl)isoindolin-1-one (Peak 2, retention time=1.425 min) (12 mg, 24% yield). ¹H NMR (400 MHz, methanol-d₄) δ 8.51 (s, 1H), 8.08 (s, 1H), 7.94 (s, 1H), 7.87-7.85 (m, 1H), 7.39-7.32 (m, 5H), 6.90-6.85 (m, 2H), 6.82 (d, J=8.0 Hz, 1H), 5.73 (d, J=46.0 Hz, 1H), 5.72 (d, J=6.4 Hz, 1H), 5.30-5.27 (m, 1H), 5.24-5.21 (m, 1H), 5.04-4.93 (m, 2H), 4.93-4.91 (m, 1H), 4.33 (d, J=14.4 Hz, 1H), 3.36-3.31 (m, 1H), 2.79 (d, J=11.6 Hz, 1H), 2.76-2.64 (m, 2H), 2.38-2.32 (m, 2H), 2.31-2.29 (m, 1H), 2.28 (s, 3H), 2.18-2.05 (m, 2H), 1.01 (d, J=6.4 Hz, 3H), 0.98 (d, J=6.4 Hz, 3H). LCMS: [M+H]⁺=663.2.

### Example 67: Compound 261 and Compound 262

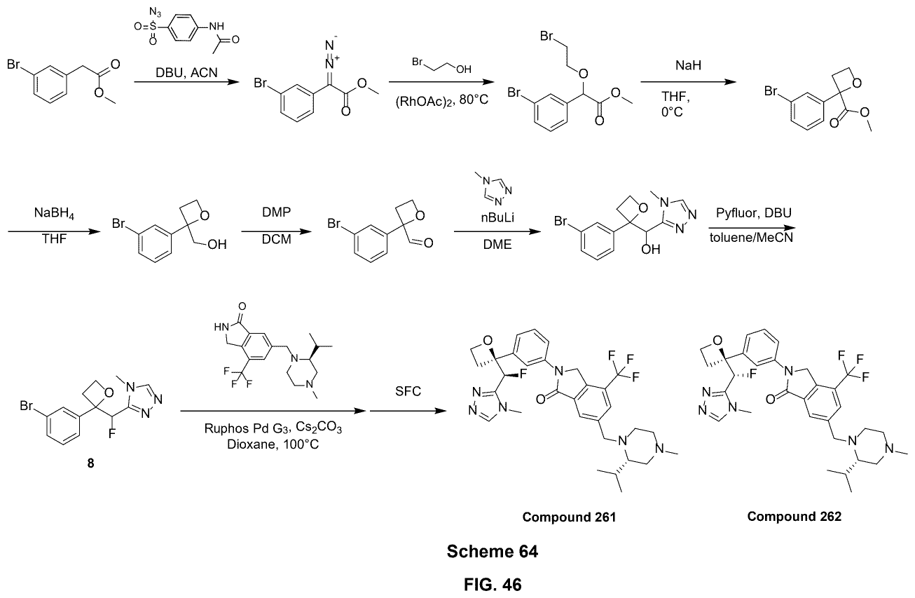

Compounds 261 and 262 can be synthesized according to Scheme 64, FIG. 46. Each isomer has been resolved whereas an absolute assignment of the respective stereochemistries has not been.

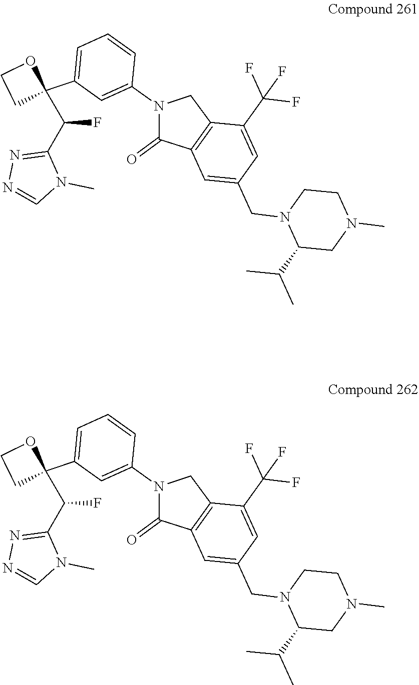 [SMILES: CC(C)[C@H]1CN(C)CCN1Cc1cc2c(c(C(F)(F)F)c1)CN(c1cccc([C@]3([C@@H](F)c4nncn4C)CCO3)c1)C2=O ; CC(C)[C@H]1CN(C)CCN1Cc1cc2c(c(C(F)(F)F)c1)CN(c1cccc([C@]3([C@H](F)c4nncn4C)CCO3)c1)C2=O]

A first intermediate, methyl 2-(3-bromophenyl)-2-diazoacetate, can be synthesized as follows. To a solution of methyl 2-(3-bromophenyl)acetate (30 g, 130.9 mmol) and 4-acetamidobenzenesulfonyl azide (47.2 g, 196.4 mmol) in acetonitrile (300 mL) was added 1,8-diazabicyclo[5.4.0]undec-7-ene (29.4 mL, 196.5 5 mmol). The mixture was stirred for 16 h at 25° C. and quenched by addition of saturated aqueous ammonium chloride (100 mL). The resulting mixture was extracted with ethyl acetate (3×100 mL). The combined organic layers were dried over anhydrous sodium sulfate and concentrated under reduced pressure. The residue was purified by silica gel chromatography (mobile phase: ethyl acetate/petroleum ether, gradient 0% to 5%) to afford methyl 2-(3-bromophenyl)-2-diazoacetate (30 g, 90% yield) as a yellow solid.

A second intermediate, methyl 2-(2-bromoethoxy)-2-(3-bromophenyl)acetate, can be synthesized as follows. To a solution of 2-bromoethanol (10 mL, 141.1 mmol) and methyl 2-(3-bromophenyl)-2-diazoacetate (30 g, 117.6 mmol) in toluene (250 mL) was added Rhodium (II) acetatedimer (129.3 mg, 0.29 mmol). The reaction mixture was stirred at 80° C. for 1 h and quenched with water (200 mL). The resulting solution was extracted with ethyl acetate (3×200 mL). The combined organic layers were dried over anhydrous sodium sulfate and concentrated under reduced pressure. The residue was purified by silica gel chromatography (mobile phase: ethyl acetate/petroleum ether, gradient 0% to 5%) to afford methyl 2-(2-bromoethoxy)-2-(3-bromophenyl)acetate (35 g, 85% yield) as a colorless oil. ¹H NMR (400 MHz, CDCl₃) δ 7.66 (s, 1H), 7.52 (d, J=8.0 Hz, 1H), 7.43 (d, J=7.6 Hz, 1H), 7.31-7.29 (m, 1H), 4.97 (s, 1H), 3.96-3.93 (m, 1H), 3.81-3.79 (m, 1H), 3.78 (s, 3H), 3.57-3.54 (m, 2H).

A third intermediate, methyl 2-(3-bromophenyl)oxetane-2-carboxylate, can be synthesized as follows. To a mixture of sodium hydride (60%, 4.4 g, 110.8 mmol) in N,N-dimethyl formamide (100 mL) and tetrahydrofuran (600 mL) was added a solution of methyl 2-(2-bromoethoxy)-2-(3-bromophenyl)acetate (30.0 g, 85.2 mmol) in N,N-dimethyl formamide (100 mL) dropwise at 0° C. The reaction mixture was stirred for 1 h at 0° C. and then 25° C. for another 1 h. The reaction was quenched by addition of saturated aqueous ammonium chloride (30 mL) and water (500 mL). The resulting solution was extracted with ethyl acetate (3×150 mL). The combined the organic phases were washed with brine (100 mL) and concentrated under reduced pressure. The residue was purified by silica gel chromatography (mobile phase: ethyl acetate/petroleum ether, gradient 0% to 10%) to afford methyl 2-(3-bromophenyl)oxetane-2-carboxylate (20 g, 87% yield) as a yellow oil.

A fourth intermediate, (2-(3-bromophenyl)oxetan-2-yl)methanol, can be synthesized as follows. To a solution of methyl 2-(3-bromophenyl)oxetane-2-carboxylate (20 g, 73.8 mmol) in tetrahydrofuran (500 mL) was added sodium borohydride (5.58 g, 147.5 mmol). The reaction mixture was stirred at 25° C. for 2 h and then quenched by addition of saturated aqueous ammonium chloride (100 mL). The resulting solution was extracted with ethyl acetate (3×100 mL). The combined organic layers were concentrated under reduced pressure. The residue was purified by silica gel chromatography (mobile phase: ethyl acetate/petroleum ether, gradient 0% to 25%) to afford (2-(3-bromophenyl)-oxetan-2-yl)methanol (17 g, 95% yield) as a light yellow oil. ¹H NMR (400 MHz, methanol-d₄) δ 7.51-7.50 (m, 1H), 7.43-7.40 (m, 1H), 7.25-7.19 (m, 2H), 4.58-4.54 (m, 2H), 3.64-3.57 (m, 2H), 3.26-3.23 (m, 1H), 2.90-2.89 (m, 1H), 2.58-2.55 (m, 1H).

A fifth intermediate, 2-(3-bromophenyl)oxetane-2-carbaldehyde, can be synthesized as follows. To a solution of (2-(3-bromophenyl)oxetan-2-yl)methanol (11.0 g, 45.3 mmol) in dichloromethane (200 mL) was added Dess-Martin periodinane (21.1 g, 49.8 mmol) in portions at 0° C. The mixture was stirred at 25° C. for 6 h and filtered through a pad of Celite. The filtrate was concentrated under reduced pressure and the residue was purified by silica gel chromatography (mobile phase: ethyl acetate/petroleum ether, gradient 0% to 20%) to afford 2-(3-bromophenyl)oxetane-2-carbaldehyde (5 g, 46% yield) as a colorless oil. ¹H NMR (400 MHz, CDCl₃) δ 9.61 (s, 1H), 7.54-7.53 (m, 1H), 7.45-7.42 (m, 1H), 7.25-7.23 (m, 2H), 4.68-4.61 (m, 2H), 3.27-3.20 (m, 1H), 2.76-2.70 (m, 1H).

A sixth intermediate, (2-(3-bromophenyl)oxetan-2-yl)(4-methyl-4H-1,2,4-triazol-3-yl)methanol, can be synthesized as follows. To a solution of 4-methyl-4H-1,2,4-triazole (2.6 g, 31.1 mmol) in 1,2-dimethoxyethane (200 mL) was added n-butyllithium (2.5 M in hexane, 12.4 mL, 31.1 mmol) at −50° C. over 5 minutes. The resulting mixture was stirred for 1 h at −50° C. and then a solution of 2-(3-bromophenyl)oxetane-2-carbaldehyde (5.0 g, 20.7 mmol) in 1,2-dimethoxyethane (10 mL) was added dropwise. The reaction mixture was allowed to warm to 0° C. over 1 h then quenched with water (3 mL). The resulting solution was concentrated under reduced pressure. The residue was purified by silica gel chromatography (mobile phase: methanol/dichloromethane, gradient 0% to 10%) to afford (2-(3-bromophenyl)oxetan-2-yl)(4-methyl-4H-1,2,4-triazol-3-yl)methanol (3.8 g, 57% yield) as a white solid. LCMS [M+H]⁺=324.1&326.1.

A seventh intermediate, 3-((2-(3-bromophenyl)oxetan-2-yl)fluoromethyl)-4-methyl-4H-1,2,4-triazole, can be synthesized as follows. To a solution of (2-(3-bromophenyl)oxetan-2-yl)(4-methyl-4H-1,2,4-triazol-3-yl)methanol (1.5 g, 4.63 mmol) in tetrahydrofuran (15 mL) and acetonitrile (5 mL) was added pyridine-2-sulfonyl fluoride (820 mg, 5.10 mmol) and 1,8-diazabicyclo[5.4.0]undec-7-ene (1.4 mL, 9.25 mmol). The result mixture was stirred at 20° C. for 2 h and then at 80° C. for 1 h. The reaction mixture was concentrated under reduced pressure and the residue was purified by silica gel chromatography (mobile phase: methanol/dichloromethane, gradient 0% to 10%) to afford 3-((2-(3-bromophenyl)oxetan-2-yl)fluoromethyl)-4-methyl-4H-1,2,4-triazole (50 mg, 20% yield) as a yellow solid. LCMS [M+H]⁺=326.1 and 328.1.

In a glove box, a mixture of 3-((2-(3-bromophenyl)oxetan-2-yl)fluoromethyl)-4-methyl-4H-1,2,4-triazole (300 mg, 0.92 mmol), (S)-6-((2-isopropyl-4-methylpiperazin-1-yl)methyl)-4-(trifluoromethyl)isoindolin-1-one (Intermediate S; 359 mg, 1.01 mol), cesium carbonate (899 mg, 1.76 mmol) and (2-Dicyclohexylphosphino-2′,6′-diisopropoxy-1, 1′-biphenyl)[2-(2′-amino-1,1′biphenyl)]palladium(II) methanesulfonate (77 mg, 0.09 mmol, CAS #1445085-77-7) in 1,4-dioxane (20 mL) was stirred at 100° C. for 16 h and concentrated under reduced pressure. The residue was purified by RP-HPLC (44% to 74% ACN/0.05% NH₄OH in water in water)) to afford 2-(3-(2-(fluoro(4-methyl-4H-1,2,4-triazol-3-yl)methyl)oxetan-2-yl)phenyl)-6-(((S)-2-isopropyl-4-methylpiperazin-1-yl)methyl)-4-(trifluoromethyl)isoindolin-1-one (50 mg, 9% yield) as a white solid.

The above mixture was further purified by chiral SFC (Column=SFC-12; Column dimensions=250 mm×30 mm×10 μm; Detection wavelength=220 nM; Flow rate=70 mL/min; Mobile phase: A: CO₂ B: ethanol (0.05% DEA); Isocratic: 35% B) with 0.1% ammonium hydroxide) to afford tentatively assigned compounds, characterized as follows:

2-(3-((S)-2-((S)-fluoro(4-methyl-4H-1,2,4-triazol-3-yl)methyl)oxetan-2-yl)phenyl)-6-(((S)-2-isopropyl-4-methylpiperazin-1-yl)methyl)-4-(trifluoromethyl)isoindolin-1-one (Peak1, retention time=1.429 min) (15 mg, 3% yield). ¹H NMR (400 MHz, methanol-d₄) δ 8.43 (s, 1H), 8.11 (s, 1H), 7.96 (s, 1H), 7.88-7.85 (m, 1H), 7.77-7.76 (m, 1H), 7.50-7.46 (m, 1H), 7.13 (d, J=8.0 Hz, 1H), 6.00 (d, J=44.0 Hz, 1H), 5.14 (s, 2H), 4.57-4.52 (m, 1H), 4.47-4.42 (m, 1H), 4.35 (d, J=14.0 Hz, 1H), 3.54-3.49 (m, 1H), 3.42 (s, 3H), 3.37-3.35 (m, 1H), 2.98-2.90 (m, 1H), 2.86-2.83 (m, 1H), 2.77-2.71 (m, 2H), 2.40-2.30 (m, 6H), 2.23-2.15 (m, 2H), 1.03 (d, J=6.4 Hz, 3H), 1.00 (d, J=6.8 Hz, 3H). LCMS: [M+H]⁺=601.3.

2-(3-((S)-2-((R)-fluoro(4-methyl-4H-1,2,4-triazol-3-yl)methyl)oxetan-2-yl)phenyl)-6-(((S)-2-isopropyl-4-methylpiperazin-1-yl)methyl)-4-(trifluoromethyl)isoindolin-1-one (Peak2, retention time=1.442 min) (15 mg, 3% yield). ¹H NMR (400 MHz, methanol-d₄) δ 8.42 (s, 1H), 8.10 (s, 1H), 7.95 (s, 1H), 7.87-7.85 (m, 1H), 7.76-7.75 (m, 1H), 7.49-7.45 (m, 1H), 7.12 (d, J=8.0 Hz, 1H), 5.99 (d, J=44.4 Hz, 1H), 5.13 (s,2H), 4.55-4.51 (m, 1H), 4.45-4.41 (m, 1H), 4.35 (d, J=14.0 Hz, 1H), 3.53-3.48 (m, 1H), 3.41 (s, 3H), 3.37-3.35 (m, 1H), 2.96-2.89 (m, 1H), 2.85-2.82 (m, 1H), 2.76-2.70 (m, 2H), 2.38-2.29 (m, 6H), 2.23-2.14 (m, 2H), 1.02 (d, J=6.4 Hz, 3H), 0.98 (d, J=6.8 Hz, 3H). LCMS: [M+H]⁺=601.3.

### Example 68: Compound 263 and Compound 264

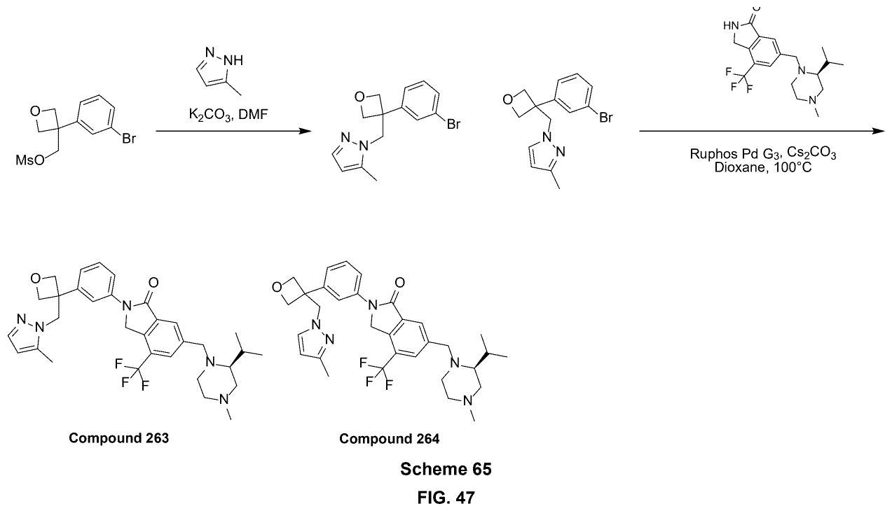

Compounds 263 ((S)-6-((2-isopropyl-4-methylpiperazin-1-yl)methyl)-2-(3-(3-((5-methyl-1H-pyrazol-1-yl)methyl)oxetan-3-yl)phenyl)-4-(trifluoromethyl)isoindolin-1-one) and 264 ((S)-6-((2-isopropyl-4-methylpiperazin-1-yl)methyl)-2-(3-(3-((3-methyl-1H-pyrazol-1-yl)methyl)oxetan-3-yl)phenyl)-4-(trifluoromethyl)isoindolin-1-one) can be synthesized according to Scheme 65, FIG. 47.

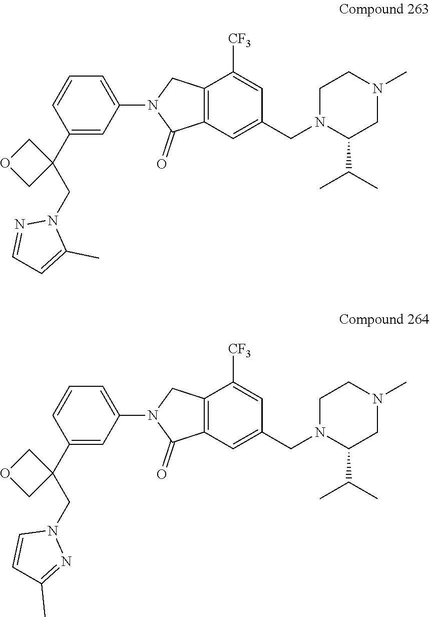 [SMILES: Cc1ccnn1CC1(c2cccc(N3Cc4c(cc(CN5CCN(C)C[C@@H]5C(C)C)cc4C(F)(F)F)C3=O)c2)COC1 ; Cc1ccn(CC2(c3cccc(N4Cc5c(cc(CN6CCN(C)C[C@@H]6C(C)C)cc5C(F)(F)F)C4=O)c3)COC2)n1]

A first pair of intermediates, 1-((3-(3-bromophenyl)oxetan-3-yl)methyl)-5-methyl-1H-pyrazole and 1-((3-(3-bromophenyl)oxetan-3-yl)methyl)-3-methyl-1H-pyrazole, can be synthesized as follows. A solution of (3-(3-bromophenyl)oxetan-3-yl)methyl methanesulfonate (1.0 g, 3.11 mmol), potassium carbonate (861 mg, 6.23 mmol) and 5-methyl-1H-pyrazole (281 mg, 3.42 mmol) in N,N-Dimethylformamide (20 mL) was stirred at 100° C. for 2 h and filtered. The filtrate was concentrated under reduced pressure and the residue was purified first by RP-HPLC (33% to 63% ACN/0.225% formic acid in water)), then by chiral SFC (Column=CAS-SH-ANA-SFC-G; Column dimensions=250 mm×30 mm×10 μm; Detection wavelength=220 nM; Flow rate=2.5 mL/min; Run time=6 min; Column temperature=40° C.) with 0.05% DEA—40% ethanol—carbon dioxide) to give isomers, characterized as follows:

1-((3-(3-bromophenyl)oxetan-3-yl)methyl)-3-methyl-1H-pyrazole (Peak 1, retention time=2.528 min) (160 mg, 16.6% yield) as a colorless oil. ¹H NMR (400 MHz, methanol-d₄) δ 7.37 (d, J=2.0 Hz, 1H), 7.21-7.17 (m, 1H), 6.97 (d, J=2.0 Hz, 1H), 6.91 (d, J=2.0 Hz, 1H), 6.85 (d, J=8.0 Hz, 1H), 5.90 (s, 2H), 5.01-4.91 (m, 4H), 4.59 (s, 2H), 2.20 (s, 3H).

1-((3-(3-bromophenyl)oxetan-3-yl)methyl)-5-methyl-1H-pyrazole (Peak 2, retention time=3.044 min) (55 mg, 8.0% yield) as a colorless oil. ¹H NMR (400 MHz, methanol-d₄) δ 7.41-7.37 (m, 2H), 7.17-7.14 (m, 1H), 6.81 (d, J=2.0 Hz 1H), 6.72 (d, J=8.0 Hz, 1H), 5.85 (s, 2H), 5.15-4.93 (m, 4H), 4.63 (s, 2H), 1.47 (s, 3H).

In a glove box, a mixture of the 1-((3-(3-bromophenyl)oxetan-3-yl)methyl)-5-methyl-1H-pyrazole (50 mg, 0.16 mmol), (S)-6-((2-isopropyl-4-methylpiperazin-1-yl)methyl)-4-(trifluoromethyl)isoindolin-1-one (Intermediate S; 58 mg, 0.16 mmol), cesium carbonate (159 mg, 0.49 mmol) and (2-dicyclohexylphosphino-2′,6′-diisopropoxy-1,1′-biphenyl)[2-(2′-amino-1,1′biphenyl)]palladium(II) methanesulfonate (13.6 mg, 0.02 mmol, CAS #: 1445085-77-7) in 1,4-dioxane (5 mL) was stirred at 100° C. for 6 h and concentrated under reduced pressure. The residue was purified by RP-HPLC (53 to 83% ACN/(0.05% NH₃·H₂O+10 mM NH₄HCO₃ in water)) to afford Compound 263: (S)-6-((2-isopropyl-4-methylpiperazin-1-yl)methyl)-2-(3-(3-((5-methyl-1H-pyrazol-1-yl)methyl)oxetan-3-yl)phenyl)-4-(trifluoromethyl)-isoindolin-1-one (34 mg, 35% yield). ¹H NMR (400 MHz, methanol-d₄) δ 8.07 (s, 1H), 7.94-7.90 (m, 2H), 7.40-7.30 (m, 2H), 6.93 (s, 1H), 6.72 (d, J=8.0 Hz, 1H), 5.84 (s, 1H), 5.21 (d, J=6.4 Hz, 2H), 5.04 (d, J=6.0 Hz, 2H), 4.94 (s, 2H), 4.69 (s, 2H), 4.33 (d, J=14.4 Hz, 1H), 3.34-3.33 (m, 1H), 2.81-2.71 (m, 3H), 2.37-2.28 (m, 6H), 2.15-2.08 (m, 2H), 1.47 (s, 3H), 1.03-0.97 (m, 6H). LCMS: [M+H]⁺=582.3.

In a glove box, a mixture of the 1-((3-(3-bromophenyl)oxetan-3-yl)methyl)-3-methyl-1H-pyrazole (160 mg, 0.52 mmol), (S)-6-((2-isopropyl-4-methylpiperazin-1-yl)-methyl)-4-(trifluoromethyl)isoindolin-1-one (185 mg, 0.52 mmol), cesium carbonate (509 mg, 1.56 mmol) and (2-dicyclohexylphosphino-2′,6′-diisopropoxy-1,1′-biphenyl)[2-(2′-amino-1,1′-biphenyl)]palladium(II) methanesulfonate (43.6 mg, 0.05 mmol, CAS #: 1445085-77-7) in 1,4-dioxane (5 mL) was stirred at 100° C. for 2 h and concentrated under reduced pressure. The residue was purified by RP-HPLC (53 to 83% ACN/(0.05% NH₃·H₂O+10 mM NH₄HCO₃ in water)) to afford Compound 264: (S)-6-((2-isopropyl-4-methylpiperazin-1-yl)methyl)-2-(3-(3-((3-methyl-1H-pyrazol-1-yl)methyl)oxetan-3-yl)phenyl)-4-(trifluoromethyl)isoindolin-1-one (151 mg, 49% yield). ¹H NMR (400 MHz, methanol-d₄) δ 8.08 (s, 1H), 7.94 (s, 1H), 7.86-7.83 (m, 1H), 7.38 (t, J=8.0 Hz, 1H), 7.17 (d, J=2.0 Hz, 1H), 6.92 (d, J=2.4 Hz, 1H), 6.79 (d, J=7.6 Hz, 1H), 5.87 (d, J=2.4 Hz, 1H), 5.09-5.02 (m, 6H), 4.66 (s, 2H), 4.33 (d, J=14.4 Hz, 1H), 3.35-3.31 (m, 1H), 2.78-2.69 (m, 3H), 2.37-2.34 (m, 3H), 2.31 (s, 3H), 2.30 (s, 3H), 2.29-2.14 (m, 2H), 1.03-0.95 (m, 6H). LCMS: [M+H]⁺=582.3. LCMS: [M+H]⁺=582.3.

### Example 69: Compound 265

Compound 265 (2-(6-cyclobutyl-4-(3-((4-methyl-4H-1,2,4-triazol-3-yl)methyl)-oxetan-3-yl)pyridin-2-yl)-4-(trifluoromethyl)isoindolin-1-one) can be synthesized as follows.

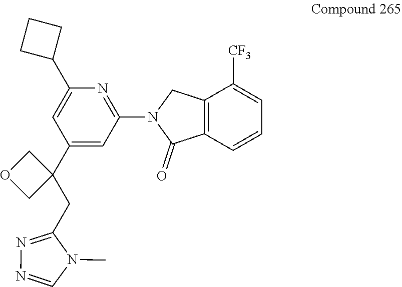 [SMILES: Cn1cnnc1CC1(c2cc(C3CCC3)nc(N3Cc4c(cccc4C(F)(F)F)C3=O)c2)COC1]

To a solution of 2-(6-chloro-4-(3-((4-methyl-4H-1,2,4-triazol-3-yl)methyl)oxetan-3-yl)pyridin-2-yl)-4-(trifluoromethyl)isoindolin-1-one (made similarly to the intermediates in Scheme 46, FIG. 28) (20 mg, 0.043 mmol, 1 eq) and tetrakis(triphenylphospine)palladium(0) (5.0 mg, 0.0043 mmol, 0.1 eq) in tetrahydrofuran (0.22 mL) was added cyclopropylzinc bromide (0.17 mL of a 0.5 M solution in THF, 0.086 mmol, 2 eq) and the reaction was stirred at room temperature for 1.5 h at which point it was heated to 60° C. and stirred for 22 h.

The solution was poured into saturated aqueous sodium bicarbonate and extracted three times with methylene chloride. The combined extracts were dried over sodium sulfate, concentrated, and purified by preparative HPLC to give 2-(6-cyclobutyl-4-(3-((4-methyl-4H-1,2,4-triazol-3-yl)methyl)oxetan-3-yl)pyridin-2-yl)-4-(trifluoromethyl)isoindolin-1-one (7.5 mg, 36%). ¹H NMR (400 MHz, DMSO-d₆) δ 8.23 (s, 1H), 8.14 (d, J=1.4 Hz, 1H), 8.12-8.04 (m, 2H), 7.85-7.76 (m, 1H), 6.80 (d, J=1.4 Hz, 1H), 5.28 (d, J=1.8 Hz, 2H), 4.96 (d, J=6.2 Hz, 2H), 4.85 (d, J=6.2 Hz, 2H), 3.57 (d, J=5.6 Hz, 3H), 3.18 (s, 3H), 2.32-2.16 (m, 4H), 2.08-1.81 (m, 2H). LCMS: 484.1 [M+H]⁺.

### Example 70: Compound 266

Compound 266 ((S)-2-(3-(3-((4-(difluoromethyl)-4H-1,2,4-triazol-3-yl)methyl)-oxetan-3-yl)phenyl)-6-((2-isopropyl-4-methylpiperazin-1-yl)methyl)-4-(trifluoromethyl)-isoindolin-1-one) can be synthesized as follows.

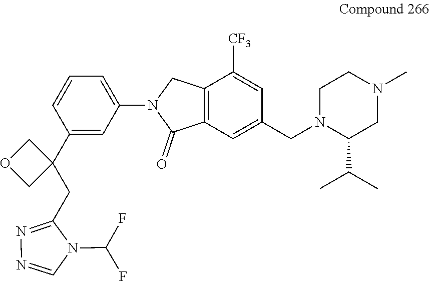 [SMILES: CC(C)[C@H]1CN(C)CCN1Cc1cc2c(c(C(F)(F)F)c1)CN(c1cccc(C3(Cc4nncn4C(F)F)COC3)c1)C2=O]

A first intermediate, (3-(3-bromophenyl)oxetan-3-yl)(1-((2-(trimethylsilyl)ethoxy)-methyl)-1H-1,2,4-triazol-5-yl)methanol, can be synthesized as follows. To a −78° C. solution of 1-((2-(trimethylsilyl)ethoxy)methyl)-1H-1,2,4-triazole (413 mg, 1.97 mmol, 1 eq) in THF (7.8 mL, 0.25 M) was added n-butyllithium (1.2 mL of 1.6 M solution in hexanes, 3.08 mmol, 1 eq). The solution was stirred briefly at −78° C. and then warmed to −40° C. After 1 h, 3-(3-bromophenyl)oxetane-3-carbaldehyde (500 mg, 1.97 mmol, 1 eq) was added in THF (1 mL) and the solution was stirred at −40° C. for 1 h. After 1 h, the reaction was poured into saturated aqueous sodium bicarbonate and extracted three times with methylene chloride. The combined extracts were dried over sodium sulfate, concentrated and purified by silica column chromatography using a methylene chloride/methanol gradient to give (3-(3-bromo-phenyl)oxetan-3-yl)(1-((2-(trimethylsilyl)ethoxy)methyl)-1H-1,2,4-triazol-5-yl)methanol (703 mg, 81%).

A second intermediate, 5-((3-(3-bromophenyl)oxetan-3-yl)chloromethyl)-1-((2-(trimethylsilyl)ethoxy)methyl)-1H-1,2,4-triazole, can be synthesized as follows. To a 0° C. solution of (3-(3-bromophenyl)oxetan-3-yl)(1-((2-(trimethylsilyl)ethoxy)methyl)-1H-1,2,4-triazol-5-yl)methanol (250 mg, 0.57 mmol, 1 eq) in methylene chloride (3.8 mL) was added pyridine (0.46 mL, 5.6 mmol, 10 eq) followed by thionyl chloride (0.21 mL, 2.8 mmol, 5 eq). The reaction was stirred at 0° C. for 30 min and subsequently warmed to room temperature and stirred for a further 1.5 h. The solution was carefully quenched with saturated aqueous sodium bicarbonate, poured into saturated aqueous sodium bicarbonate, and extracted three times with methylene chloride. The combined extracts were dried over sodium sulfate, concentrated, and purified by silica column chromatography using a heptanes/isopropyl acetate gradient to give 5-((3-(3-bromophenyl)oxetan-3-yl)chloromethyl)-1-((2-(trimethyl-silyl)ethoxy)methyl)-1H-1,2,4-triazole (237 mg, 91%).

A third intermediate, 5-((3-(3-bromophenyl)oxetan-3-yl)methyl)-1-((2-(trimethyl-silyl)ethoxy)methyl)-1H-1,2,4-triazole, can be synthesized as follows. To a solution of 5-((3-(3-bromophenyl)oxetan-3-yl)chloromethyl)-1-((2-(trimethylsilyl)ethoxy)methyl)-1H-1,2,4-triazole (237 mg, 0.52 mmol, 1 eq) in acetic acid (3.4 mL) was added zinc (169 mg, 2.6 mmol, 5 eq). The suspension was stirred at room temperature for 5 h at which point it was filtered and the filtrate was poured into saturated aqueous sodium bicarbonate. The solution was extracted three times with methylene chloride and the combined extracts were dried over sodium sulfate, concentrated and purified by silica column chromatography using a heptanes/isopropyl acetate gradient to give 5-((3-(3-bromophenyl)oxetan-3-yl)methyl)-1-((2-(trimethylsilyl)ethoxy)methyl)-1H-1,2,4-triazole (199 mg, 91%).

A fourth intermediate, 3-((3-(3-bromophenyl)oxetan-3-yl)methyl)-4H-1,2,4-triazole, can be synthesized as follows. To a solution of 5-((3-(3-bromophenyl)oxetan-3-yl)methyl)-1-((2-(trimethylsilyl)ethoxy)methyl)-1H-1,2,4-triazole (199 mg, 0.47 mmol, 1 eq) in methylene chloride (2.3 mL) was added trifluoroacetic acid (0.71 mL, 9.4 mmol, 20 eq). The solution was stirred at room temperature for 3 h at which point an additional portion of trifluoroacetic acid (0.3 mL, 4.0 mmol, 8.6 eq) was added. The solution was stirred 22 h at room temperature and poured into saturated aqueous sodium bicarbonate. The solution was extracted three times with methylene chloride and the combined extracts were dried over sodium sulfate, concentrated and purified by silica column chromatography using a methylene chloride/methanol gradient to give 3-((3-(3-bromophenyl)oxetan-3-yl)methyl)-4H-1,2,4-triazole (93 mg, 67%).

A fifth intermediate, 3-((3-(3-bromophenyl)oxetan-3-yl)methyl)-4-(difluoromethyl)-4H-1,2,4-triazole, can be synthesized as follows. To a vial containing 3-((3-(3-bromophenyl)oxetan-3-yl)methyl)-4H-1,2,4-triazole (93 mg, 0.32 mmol, 1 eq), sodium chlorodifluoroacetate (62.7 mg, 0.41 mmol, 1.3 eq) and potassium carbonate (50 mg, 0.51 mmol, 1.6 eq) was added acetonitrile (2.1 mL) and the reaction was heated to 90° C. for 1.5 h. After 1.5 h an additional portion of sodium chlorodifluoroacetate (62.7 mg, 0.41 mmol, 1.3 eq) and potassium carbonate (50 mg, 0.51 mmol, 1.6 eq) was added and the reaction was stirred an additional 2.5 h at 90° C. The solution was poured into saturated aqueous sodium bicarbonate and extracted three times with methylene chloride. The combined extracts were dried over sodium sulfate, concentrated, and purified by preparative HPLC to give 3-((3-(3-bromophenyl)oxetan-3-yl)methyl)-4-(difluoromethyl)-4H-1,2,4-triazole (17 mg, 16%).

To form Compound 266, a vial containing 3-((3-(3-bromophenyl)oxetan-3-yl)methyl)-4-(difluoromethyl)-4H-1,2,4-triazole (15 mg, 0.044 mmol, 1 eq), (S)-6-((2-isopropyl-4-methylpiperazin-1-yl)methyl)-4-(trifluoromethyl)isoindolin-1-one (23.2 mg, 0.065 mmol, 1.5 eq), Me₄^{t}BuXPhos Pd G3 (2.0 mg, 0.0022 mmol, 0.05 eq), Me₄^{t}BuXPhos (2.2 mg, 0.0044 mmol, 0.10 eq), cesium carbonate (31 mg, 0.10 mmol, 2.2 eq) was added t-butanol (0.44 mL). The reaction was heated at 100° C. for 6 h at which point it was poured into saturated aqueous sodium bicarbonate and extracted three times with methylene chloride. The combined extracts were dried over sodium sulfate, concentrated, and purified by preparative HPLC to give (S)-2-(3-(3-((4-(difluoromethyl)-4H-1,2,4-triazol-3-yl)methyl)-oxetan-3-yl)phenyl)-6-((2-isopropyl-4-methylpiperazin-1-yl)methyl)-4-(trifluoromethyl)-isoindolin-1-one (19.5 mg, 72%). ¹H NMR (400 MHz, DMSO-d₆) δ 8.82 (s, 1H), 7.99 (s, 1H), 7.92 (s, 1H), 7.84 (dd, J=8.0, 2.1 Hz, 1H), 7.62 (t, J=2.0 Hz, 1H), 7.51 (t, J=58.4 Hz, 1H), 7.38-7.32 (m, 1H), 6.95 (dd, J=7.2, 1.7 Hz, 1H), 5.12 (s, 2H), 4.96 (d, J=6.2 Hz, 2H), 4.92 (d, J=6.2 Hz, 2H), 4.22 (d, J=14.3 Hz, 1H), 3.73 (s, 2H), 3.50-3.18 (m, 2H), 2.63 (ddd, J=12.2, 8.0, 4.6 Hz, 2H), 2.34-2.17 (m, 3H), 2.16 (s, 3H), 2.02-1.88 (m, 2H), 0.93 (d, J=6.6 Hz, 3H), 0.88 (d, J=6.6 Hz, 3H). LCMS: 619.2 [M+H]⁺.

### Example 71: Compounds 267 and 268

Compounds 267 (2-(3-(3-((R)-(4-(difluoromethyl)-4H-1,2,4-triazol-3-yl)fluoromethyl)oxetan-3-yl)phenyl)-6-(((S)-2-isopropyl-4-methylpiperazin-1-yl)methyl)-4-(trifluoromethyl)isoindolin-1-one) and 268 2-(3-(3-((S)-(4-(difluoromethyl)-4H-1,2,4-triazol-3-yl)fluoromethyl)oxetan-3-yl)phenyl)-6-(((S)-2-isopropyl-4-methylpiperazin-1-yl)methyl)-4-(trifluoromethyl)isoindolin-1-one can be synthesized as follows.

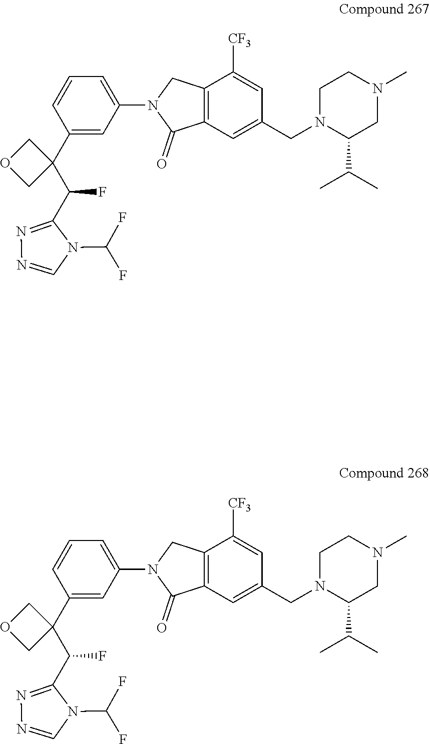 [SMILES: CC(C)[C@H]1CN(C)CCN1Cc1cc2c(c(C(F)(F)F)c1)CN(c1cccc(C3([C@@H](F)c4nncn4C(F)F)COC3)c1)C2=O ; CC(C)[C@H]1CN(C)CCN1Cc1cc2c(c(C(F)(F)F)c1)CN(c1cccc(C3([C@H](F)c4nncn4C(F)F)COC3)c1)C2=O]

A first intermediate, 5-((3-(3-bromophenyl)oxetan-3-yl)fluoromethyl)-1-((2-(trimethylsilyl)ethoxy)methyl)-1H-1,2,4-triazole, can be synthesized as follows. To a −78° C. solution of (3-(3-bromophenyl)oxetan-3-yl)(1-((2-(trimethylsilyl)ethoxy)methyl)-1H-1,2,4-triazol-5-yl)methanol (703 mg, 1.6 mmol, 1 eq) in methylene chloride (16.0 mL) was added diethylaminosulfur trifluoride (1.76 mL of a 1M solution in methylene chloride, 1.76 mmol, 1.1 eq). The reaction was warmed to 0° C. after 20 min and stirred for 1 h. After 1 h the reaction was poured into saturated aqueous sodium bicarbonate and extracted three times with methylene chloride. The combined extracts were dried over sodium sulfate, concentrated and purified by silica column chromatography using a methylene chloride/methanol gradient to give 5-((3-(3-bromophenyl)oxetan-3-yl)fluoromethyl)-1-((2-(trimethylsilyl)ethoxy)methyl)-1H-1,2,4-triazole (666 mg, 94%).

A second intermediate, 3-((3-(3-bromophenyl)oxetan-3-yl)fluoromethyl)-4H-1,2,4-triazole, can be synthesized as follows. To a solution of 5-((3-(3-bromophenyl)oxetan-3-yl)fluoromethyl)-1-((2-(trimethylsilyl)ethoxy)methyl)-1H-1,2,4-triazole (666 mg, 1.51 mmol, 1 eq) in methylene chloride (7.6 mL) was added trifluoroacetic acid (1.7 mL). The reaction was stirred at room temperature for 4 h at which point the solution was carefully poured into saturated aqueous sodium bicarbonate and extracted three times with methylene chloride. The combined extracts were dried over sodium sulfate, concentrated and purified by silica column chromatography using a methylene chloride/methanol gradient to give 3-((3-(3-bromophenyl)oxetan-3-yl)fluoromethyl)-4H-1,2,4-triazole (322 mg, 69%).

A third intermediate, 3-((3-(3-bromophenyl)oxetan-3-yl)fluoromethyl)-4-(difluoro-methyl)-4H-1,2,4-triazole, can be synthesized as follows. To a solution of 3-((3-(3-bromophenyl)oxetan-3-yl)fluoromethyl)-4H-1,2,4-triazole (333 mg, 1.07 mmol, 1 eq) in acetonitrile (7.1 mL) was added sodium chlorodifluoroacetate (211 mg, 1.4 mmol, 1.3 eq) and potassium carbonate (170 mg, 1.7 mmol, 1.6 eq). The reaction was heated to 100° C. for 7.5 h. The solution was poured into saturated aqueous sodium bicarbonate, extracted three times with methylene chloride. The combined extracts were dried over sodium sulfate, concentrated, and purified by preparative HPLC to give 3-((3-(3-bromophenyl)oxetan-3-yl)fluoromethyl)-4-(difluoromethyl)-4H-1,2,4-triazole (24.5 mg, 6.3%).

A fourth intermediate, 2-(3-(3-((4-(difluoromethyl)-4H-1,2,4-triazol-3-yl)fluoromethyl)oxetan-3-yl)phenyl)-6-(((S)-2-isopropyl-4-methylpiperazin-1-yl)methyl)-4-(trifluoromethyl)isoindolin-1-one, can be synthesized as follows. To a vial containing 3-((3-(3-bromophenyl)oxetan-3-yl)fluoromethyl)-4-(difluoromethyl)-4H-1,2,4-triazole (24.5 mg, 0.069 mmol, 1 eq), (S)-6-((2-isopropyl-4-methylpiperazin-1-yl)methyl)-4-(trifluoromethyl)-isoindolin-1-one (Intermediate S; 37 mg, 0.10 mmol, 1.5 eq), Me₄^{t}BuXPhos Pd G3 (3.1 mg, 0.0035 mmol, 0.05 eq), Me₄^{t}BuXPhos (3.5 mg, 0.0069 mmol, 0.10 eq), cesium carbonate (49.5 mg, 0.15 mmol, 2.2 eq) was added t-butanol (0.69 mL). The reaction was heated at 100° C. for 20 h at which point it was poured into saturated aqueous sodium bicarbonate and extracted three times with methylene chloride. The combined extracts were dried over sodium sulfate, concentrated, and purified by preparative HPLC to give 2-(3-(3-((4-(difluoromethyl)-4H-1,2,4-triazol-3-yl)fluoromethyl)oxetan-3-yl)phenyl)-6-(((S)-2-isopropyl-4-methylpiperazin-1-yl)methyl)-4-(trifluoromethyl)isoindolin-1-one (14 mg, 32%).

Separation of the products of the prior step gives 2-(3-(3-((R)-(4-(difluoromethyl)-4H-1,2,4-triazol-3-yl)fluoromethyl)oxetan-3-yl)phenyl)-6-(((S)-2-isopropyl-4-methylpiperazin-1-yl)methyl)-4-(trifluoromethyl)isoindolin-1-one, and 2-(3-(3-((S)-(4-(difluoromethyl)-4H-1,2,4-triazol-3-yl)fluoromethyl)oxetan-3-yl)phenyl)-6-(((S)-2-isopropyl-4-methylpiperazin-1-yl)methyl)-4-(trifluoromethyl)isoindolin-1-one.

In a final step, 2-(3-(3-((4-(difluoromethyl)-4H-1,2,4-triazol-3-yl)fluoromethyl)-oxetan-3-yl)phenyl)-6-(((S)-2-isopropyl-4-methylpiperazin-1-yl)methyl)-4-(trifluoromethyl)-isoindolin-1-one (14 mg) was subjected to chiral SFC to give a mixture of isomers (Compounds 267, 268) 2-(3-(3-((R)-(4-(difluoromethyl)-4H-1,2,4-triazol-3-yl)fluoromethyl)oxetan-3-yl)phenyl)-6-(((S)-2-isopropyl-4-methylpiperazin-1-yl)methyl)-4-(trifluoromethyl)isoindolin-1-one, and 2-(3-(3-((S)-(4-(difluoromethyl)-4H-1,2,4-triazol-3-yl)fluoromethyl)oxetan-3-yl)phenyl)-6-(((S)-2-isopropyl-4-methylpiperazin-1-yl)methyl)-4-(trifluoromethyl)isoindolin-1-one. 5 mg of each enantiomer was obtained.

First isomer: ¹H NMR (400 MHz, DMSO-d₆) δ 8.98 (s, 1H), 7.99 (s, 1H), 7.94-7.84 (m, 2H), 7.72 (t, J=2.0 Hz, 1H), 7.63 (t, J=58.1 Hz, 1H), 7.38 (t, J=8.0 Hz, 1H), 7.10-7.03 (m, 1H), 6.42 (d, J=46.0 Hz, 1H), 5.36 (d, J=6.9 Hz, 1H), 5.24 (dd, J=6.4, 1.5 Hz, 1H), 5.20 (dd, J=6.9, 2.1 Hz, 1H), 5.11 (s, 2H), 4.83 (dd, J=6.4, 4.0 Hz, 1H), 4.22 (d, J=14.4 Hz, 1H), 3.38 (d, J=14.3 Hz, 1H), 2.63 (dt, J=13.6, 9.6 Hz, 2H), 2.52-2.48 (m, 1H), 2.35-2.17 (m, 3H), 2.14 (s, 3H), 2.02-1.86 (m, 2H), 0.93 (d, J=6.6 Hz, 3H), 0.88 (d, J=6.6 Hz, 3H). LCMS: 637.2 [M+H]⁺.

Second isomer: ¹H NMR (400 MHz, DMSO-d₆) δ 8.98 (s, 1H), 7.99 (s, 1H), 7.95-7.86 (m, 2H), 7.71 (t, J=2.0 Hz, 1H), 7.63 (t, J=58.1 Hz, 1H), 7.38 (t, J=8.0 Hz, 1H), 7.07 (d, J=7.7 Hz, 1H), 6.42 (d, J=46.1 Hz, 1H), 5.36 (d, J=6.9 Hz, 1H), 5.24 (dd, J=6.5, 1.5 Hz, 1H), 5.20 (dd, J=6.9, 2.1 Hz, 1H), 5.11 (s, 2H), 4.83 (dd, J=6.4, 4.0 Hz, 1H), 4.22 (d, J=14.4 Hz, 1H), 3.38 (d, J=14.3 Hz, 1H), 2.68-2.54 (m, 2H), 2.52-2.48 (m, 1H), 2.34-2.17 (m, 3H), 2.14 (s, 3H) 2.02-1.82 (m, 2H), 0.93 (d, J=6.6 Hz, 3H), 0.88 (d, J=6.6 Hz, 3H). LCMS: 637.3 [M+H]⁺.

### Example 72: Compound 269 and 270

Compounds 269 (2-(3-(3-((R)-fluoro(4-fluoro-1-methyl-1H-imidazol-2-yl)methyl)-oxetan-3-yl)phenyl)-6-(((S)-2-isopropyl-4-methylpiperazin-1-yl)methyl)-4-(trifluoromethyl)-isoindolin-1-one) and 270 2-(3-(3-((S)-fluoro(4-fluoro-1-methyl-1H-imidazol-2-yl)methyl)-oxetan-3-yl)phenyl)-6-(((S)-2-isopropyl-4-methylpiperazin-1-yl)methyl)-4-(trifluoromethyl)-isoindolin-1-one can be synthesized as follows.

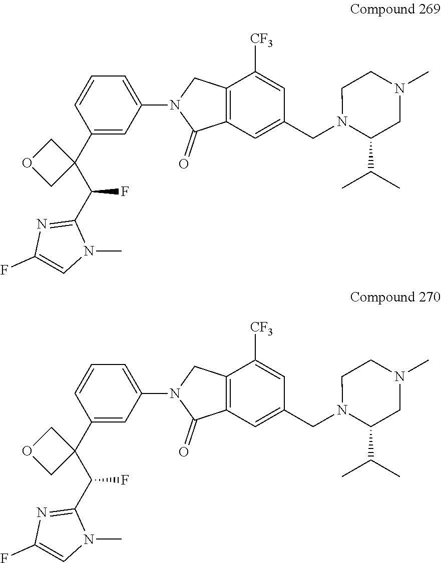 [SMILES: CC(C)[C@H]1CN(C)CCN1Cc1cc2c(c(C(F)(F)F)c1)CN(c1cccc(C3([C@@H](F)c4nc(F)cn4C)COC3)c1)C2=O ; CC(C)[C@H]1CN(C)CCN1Cc1cc2c(c(C(F)(F)F)c1)CN(c1cccc(C3([C@H](F)c4nc(F)cn4C)COC3)c1)C2=O]

A first intermediate, (3-(3-bromophenyl)oxetan-3-yl)(4-fluoro-1-methyl-1H-imidazol-2-yl)methanol, can be synthesized as follows. To a solution of 4-fluoro-1-methyl-1H-imidazole (71 mg, 0.71 mmol, 1.2 eq) in THF (2.9 mL) at −78° C. was added n-butyllithium (0.4 mL of 1.6 M solution in hexanes, 0.64 mmol, 1.1 eq). The reaction was warmed to −40° C. after 5 min and stirred at that temperature for 30 min at which point it was recooled to −78° C. and 3-(3-bromophenyl)oxetane-3-carbaldehyde (140 mg, 0.59 mmol, 1 eq) was added in a small amount of THF. The solution was stirred at −78° C. for 1 h. After 1 h the reaction was poured into saturated aqueous sodium bicarbonate and extracted three times with methylene chloride. The combined extracts were dried over sodium sulfate, concentrated and purified by silica column chromatography using a methylene chloride/methanol gradient to give (3-(3-bromophenyl)oxetan-3-yl)(4-fluoro-1-methyl-1H-imidazol-2-yl)methanol (123 mg, 62%).

A second intermediate, 2-((3-(3-bromophenyl)oxetan-3-yl)fluoromethyl)-4-fluoro-1-methyl-1H-imidazole, can be synthesized as follows. To a −78° C. solution of (3-(3-bromophenyl)oxetan-3-yl)(4-fluoro-1-methyl-1H-imidazol-2-yl)methanol (123 mg, 0.36 mmol, 1 eq) in methylene chloride (3.6 mL) was added diethylaminosulfur trifluoride (0.40 mL of a 1 M solution in methylene chloride, 0.40 mmol, 1.1 eq). The reaction was stirred briefly at −78° C. before warming to 0° C. and stirring at that temperature for 2 h. After 2 h the reaction was poured into saturated aqueous sodium bicarbonate and extracted three times with methylene chloride. The combined extracts were dried over sodium sulfate, concentrated and purified by silica column chromatography using a methylene chloride/methanol gradient to give 2-((3-(3-bromophenyl)oxetan-3-yl)fluoromethyl)-4-fluoro-1-methyl-1H-imidazole (65 mg, 62%).

A third intermediate, 2-(3-(3-(fluoro(4-fluoro-1-methyl-1H-imidazol-2-yl)methyl)oxetan-3-yl)phenyl)-6-(((S)-2-isopropyl-4-methylpiperazin-1-yl)methyl)-4-(trifluoromethyl)isoindolin-1-one, can be synthesized as follows. To a vial containing 2-((3-(3-bromophenyl)oxetan-3-yl)fluoromethyl)-4-fluoro-1-methyl-1H-imidazole (34 mg, 0.098 mmol, 1 eq), (S)-6-((2-isopropyl-4-methylpiperazin-1-yl)methyl)-4-(trifluoromethyl)isoindolin-1-one (Intermediate S; 35 mg, 0.098 mmol, 1 eq), Me₄^{t}BuXPhos Pd G3 (3.5 mg, 0.0039 mmol, 0.04 eq), Me₄^{t}BuXPhos (4.9 mg, 0.0098 mmol, 0.10 eq), K₃PO₄ (43 mg, 0.20 mmol, 2.0 eq) was added t-butanol (0.49 mL). The reaction was heated at 80° C. for 24 h at which point it was poured into saturated aqueous sodium bicarbonate and extracted three times with methylene chloride. The combined extracts were dried over sodium sulfate, concentrated, and purified by silica column chromatography using a methylene chloride/methanol gradient to give 2-(3-(3-(fluoro(4-fluoro-1-methyl-1H-imidazol-2-yl)methyl)oxetan-3-yl)phenyl)-6-(((S)-2-isopropyl-4-methylpiperazin-1-yl)methyl)-4-(trifluoromethyl)isoindolin-1-one (16 mg, 26%).

A mixture of isomers, Compound 269 2-(3-(3-((R)-fluoro(4-fluoro-1-methyl-1H-imidazol-2-yl)methyl)oxetan-3-yl)phenyl)-6-(((S)-2-isopropyl-4-methylpiperazin-1-yl)methyl)-4-(trifluoromethyl)isoindolin-1-one, and Compound 270 2-(3-(3-((S)-fluoro(4-fluoro-1-methyl-1H-imidazol-2-yl)methyl)oxetan-3-yl)phenyl)-6-(((S)-2-isopropyl-4-methylpiperazin-1-yl)methyl)-4-(trifluoromethyl)isoindolin-1-one, can be separated as follows.

2-(3-(3-(fluoro(4-fluoro-1-methyl-1H-imidazol-2-yl)methyl)oxetan-3-yl)phenyl)-6-(((S)-2-isopropyl-4-methylpiperazin-1-yl)methyl)-4-(trifluoromethyl)isoindolin-1-one (16 mg) was subjected to chiral SFC to give the isolated compounds: 2-(3-(3-((R)-fluoro(4-fluoro-1-methyl-1H-imidazol-2-yl)methyl)oxetan-3-yl)phenyl)-6-(((S)-2-isopropyl-4-methylpiperazin-1-yl)methyl)-4-(trifluoromethyl)isoindolin-1-one, and 2-(3-(3-((S)-fluoro(4-fluoro-1-methyl-1H-imidazol-2-yl)methyl)oxetan-3-yl)phenyl)-6-(((S)-2-isopropyl-4-methylpiperazin-1-yl)methyl)-4-(trifluoromethyl)isoindolin-1-one. Isolated 4.6 mg of first isomer and 4.7 mg of second isomer.

First isomer: ¹H NMR (400 MHz, DMSO-d6) δ 8.03-7.88 (m, 3H), 7.49 (t, J=2.0 Hz, 1H), 7.39 (t, J=8.0 Hz, 1H), 7.00 (dt, J=7.9, 1.3 Hz, 1H), 6.73 (dd, J=8.2, 1.6 Hz, 1H), 6.02 (dd, J=46.3, 1.3 Hz, 1H), 5.40 (d, J=6.7 Hz, 1H), 5.22-5.14 (m, 2H), 5.08 (dd, J=6.7, 2.2 Hz, 1H), 5.00 (d, J=17.5 Hz, 1H), 4.80 (dd, J=6.1, 3.4 Hz, 1H), 4.21 (d, J=14.3 Hz, 1H), 3.38 (d, J=14.3 Hz, 1H), 3.15 (s, 3H), 2.62 (t, J=11.9 Hz, 2H), 2.33-2.17 (m, 4H), 2.15 (s, 3H), 1.94 (q, J=9.6 Hz, 2H), 0.93 (d, J=6.6 Hz, 3H), 0.88 (d, J=6.6 Hz, 3H). LCMS: 618.3 [M+H]⁺.

Second isomer: ¹H NMR (400 MHz, DMSO-d6) δ 8.04-7.90 (m, 3H), 7.49 (t, J=2.0 Hz, 1H), 7.39 (t, J=8.0 Hz, 1H), 7.03-6.96 (m, 1H), 6.73 (dd, J=8.2, 1.6 Hz, 1H), 6.12-5.92 (m, 1H), 5.40 (d, J=6.6 Hz, 1H), 5.24-5.13 (m, 2H), 5.08 (dd, J=6.8, 2.2 Hz, 1H), 5.00 (d, J=17.6 Hz, 1H), 4.80 (dd, J=6.1, 3.5 Hz, 1H), 4.22 (d, J=14.4 Hz, 1H), 3.38 (d, J=14.3 Hz, 1H), 2.62 (t, J=11.3 Hz, 2H), 2.33-2.17 (m, 4H), 2.15 (s, 3H), 1.95 (q, J=10.4 Hz, 2H), 0.93 (d, J=6.6 Hz, 3H), 0.88 (d, J=6.6 Hz, 3H). LCMS: 618.3 [M+H]⁺.

### Example 73: Compound 271 and 272

Compounds 271 ((S)-2-(3-(3-((4-ethyl-4H-1,2,4-triazol-3-yl)fluoromethyl)oxetan-3-yl)phenyl)-4-(trifluoromethyl)isoindolin-1-one) and Compound 272 ((R)-2-(3-(3-((4-ethyl-4H-1,2,4-triazol-3-yl)fluoromethyl)oxetan-3-yl)phenyl)-4-(trifluoromethyl)isoindolin-1-one) can be synthesized as follows.

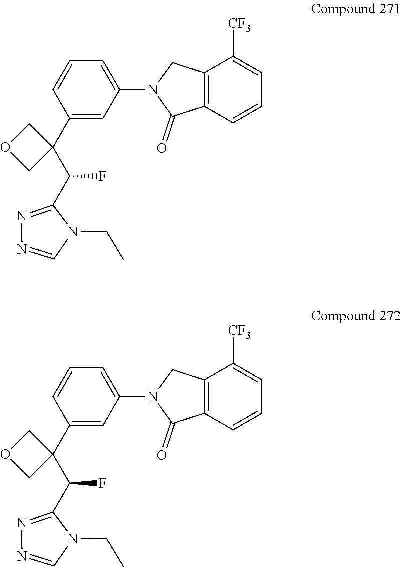 [SMILES: CCn1cnnc1[C@@H](F)C1(c2cccc(N3Cc4c(cccc4C(F)(F)F)C3=O)c2)COC1 ; CCn1cnnc1[C@H](F)C1(c2cccc(N3Cc4c(cccc4C(F)(F)F)C3=O)c2)COC1]

A first intermediate, (3-(3-bromophenyl)oxetan-3-yl)(4-ethyl-4H-1,2,4-triazol-3-yl)methanol, can be synthesized as follows. To a solution of 4-ethyl-4H-1,2,4-triazole (121 mg, 1.24 mmol, 1.2 eq) in dimethoxyethane (5.2 mL) at −40° C. was added n-butyllithium (0.71 mL of a 1.6M solution in hexanes, 1.14 mmol, 1.1 eq). The solution was stirred at −40° C. for 30 min at which point 3-(3-bromophenyl)oxetane-3-carbaldehyde (250 mg, 1.04 mmol, 1 eq) was added in a small amount of dimethoxyethane. The solution was stirred at −40° C. for 40 min. After 40 min the reaction was poured into saturated aqueous sodium bicarbonate and extracted three times with methylene chloride. The combined extracts were dried over sodium sulfate, concentrated and purified by silica column chromatography using a methylene chloride/methanol gradient to give (3-(3-bromophenyl)oxetan-3-yl)(4-ethyl-4H-1,2,4-triazol-3-yl)methanol (88 mg, 26%).

A second intermediate, 3-((3-(3-bromophenyl)oxetan-3-yl)fluoromethyl)-4-ethyl-4H-1,2,4-triazole, can be synthesized as follows. To a solution of (3-(3-bromophenyl)-oxetan-3-yl)(4-ethyl-4H-1,2,4-triazol-3-yl)methanol (146 mg, 0.43 mmol, 1 eq) in dichloromethane (2.2 mL) at −78° C. was added diethylaminosulfur trifluoride (0.52 mL of a 1 M solution in dichloromethane, 0.52 mmol, 1.2 eq) and the reaction was warmed to 0° C. After 1.5 h the reaction was poured into saturated aqueous sodium bicarbonate and extracted three times with methylene chloride. The combined extracts were dried over sodium sulfate, concentrated and purified by silica column chromatography using a methylene chloride/methanol gradient to give 3-((3-(3-bromophenyl)oxetan-3-yl)fluoro-methyl)-4-ethyl-4H-1,2,4-triazole (92 mg, 63%).

A third intermediate, 2-(3-(3-((4-ethyl-4H-1,2,4-triazol-3-yl)fluoromethyl)oxetan-3-yl)phenyl)-4-(trifluoromethyl)isoindolin-1-one, can be synthesized as follows. To a vial containing 3-((3-(3-bromophenyl)oxetan-3-yl)fluoromethyl)-4-ethyl-4H-1,2,4-triazole (45 mg, 0.13 mmol, 1 eq), 4-(trifluoromethyl)isoindolin-1-one (27 mg, 0.13 mmol, 1 eq), Me₄^{t}BuXPhos Pd G3 (4.7 mg, 0.0053 mmol, 0.04 eq), Me₄^{t}BuXPhos (6.6 mg, 0.013 mmol, 0.10 eq), K₃PO₄ (56 mg, 0.26 mmol, 2.0 eq) was added t-butanol (0.53 mL). The solution was heated to 80° C. for 21 h at which point it was poured into saturated aqueous sodium bicarbonate and extracted three times with methylene chloride. The combined extracts were dried over sodium sulfate, concentrated, and purified by silica column chromatography using a methylene chloride/methanol gradient to give a mixture of isomers: 2-(3-(3-((4-ethyl-4H-1,2,4-triazol-3-yl)fluoromethyl)oxetan-3-yl)phenyl)-4-(trifluoromethyl)isoindolin-1-one (39 mg, 64%).

Individual compounds 271 and 272, (R)-2-(3-(3-((4-ethyl-4H-1,2,4-triazol-3-yl)fluoromethyl)oxetan-3-yl)phenyl)-4-(trifluoromethyl)isoindolin-1-one and (S)-2-(3-(3-((4-ethyl-4H-1,2,4-triazol-3-yl)fluoromethyl)oxetan-3-yl)phenyl)-4-(trifluoromethyl)isoindolin-1-one, can be obtained as follows.

2-(3-(3-((4-ethyl-4H-1,2,4-triazol-3-yl)fluoromethyl)oxetan-3-yl)phenyl)-4-(trifluoro-methyl)isoindolin-1-one (39 mg) was subjected to chiral SFC to give R)-2-(3-(3-((4-ethyl-4H-1,2,4-triazol-3-yl)fluoromethyl)oxetan-3-yl)phenyl)-4-(trifluoromethyl)isoindolin-1-one and (S)-2-(3-(3-((4-ethyl-4H-1,2,4-triazol-3-yl)fluoromethyl)oxetan-3-yl)phenyl)-4-(trifluoro-methyl)isoindolin-1-one Isolated 17 mg of first isomer and 17 mg of second isomer.

First isomer: ¹H NMR (400 MHz, DMSO-d6) δ 8.47 (s, 1H), 8.08 (d, J=7.6 Hz, 1H), 8.05 (dt, J=7.6, 0.9 Hz, 1H), 7.91 (ddd, J=8.2, 2.3, 0.9 Hz, 1H), 7.83-7.77 (m, 1H), 7.62 (t, J=2.0 Hz, 1H), 7.37 (t, J=8.0 Hz, 1H), 7.07-7.00 (m, 1H), 6.26 (d, J=46.3 Hz, 1H), 5.48-5.42 (m, 1H), 5.25 (dd, J=6.1, 1.7 Hz, 1H), 5.18 (dd, J=6.8, 2.2 Hz, 1H), 5.13 (d, J=5.8 Hz, 2H), 4.82 (dd, J=6.2, 3.8 Hz, 1H), 3.74 (dd, J=14.1, 7.1 Hz, 1H), 3.67 (dd, J=14.1, 7.1 Hz, 1H), 1.02 (t, J=7.3 Hz, 3H). LCMS: 461.1 [M+H]⁺.

Second isomer: ¹H NMR (400 MHz, DMSO-d6) δ 8.47 (s, 1H), 8.08 (d, J=7.7 Hz, 1H), 8.05 (dt, J=7.8, 0.9 Hz, 1H), 7.91 (ddd, J=8.3, 2.3, 0.9 Hz, 1H), 7.83-7.77 (m, 1H), 7.62 (t, J=2.0 Hz, 1H), 7.37 (t, J=8.0 Hz, 1H), 7.04 (ddd, J=7.9, 1.8, 0.9 Hz, 1H), 6.26 (d, J=46.3 Hz, 1H), 5.45 (d, J=6.8 Hz, 1H), 5.25 (dd, J=6.2, 1.7 Hz, 1H), 5.18 (dd, J=6.8, 2.2 Hz, 1H), 5.13 (d, J=5.8 Hz, 2H), 4.82 (dd, J=6.1, 3.9 Hz, 1H), 3.74 (dd, J=14.1, 7.1 Hz, 1H), 3.67 (dd, J=14.1, 7.1 Hz, 1H), 1.02 (t, J=7.3 Hz, 3H). LCMS: 461.1 [M+H]⁺.

### Example 74: Compound 273 and 274

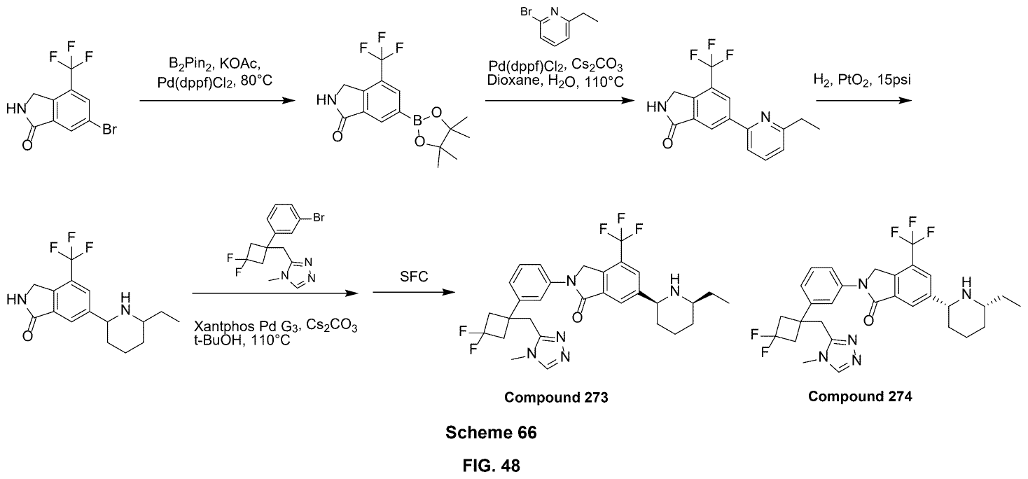

Compounds 273 (2-(3-(3,3-difluoro-1-((4-methyl-4H-1,2,4-triazol-3-yl)methyl)-cyclobutyl)phenyl)-6-((2S,6S)-6-ethylpiperidin-2-yl)-4-(trifluoromethyl)isoindolin-1-one), and 274 (2-(3-(3,3-difluoro-1-((4-methyl-4H-1,2,4-triazol-3-yl)methyl)cyclobutyl)phenyl)-6-((2R,6R)-6-ethylpiperidin-2-yl)-4-(trifluoromethyl)isoindolin-1-one) can be synthesized according to Scheme 66, FIG. 48.

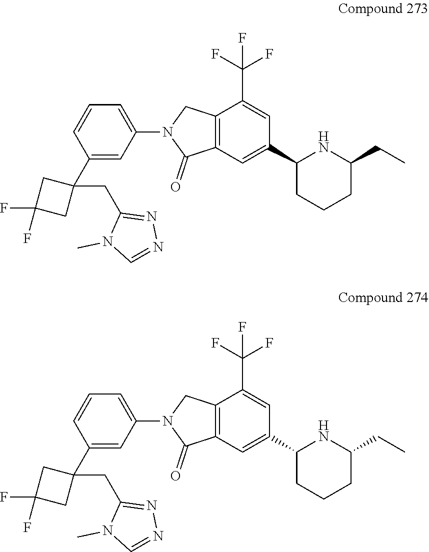 [SMILES: CC[C@H]1CCC[C@@H](c2cc3c(c(C(F)(F)F)c2)CN(c2cccc(C4(Cc5nncn5C)CC(F)(F)C4)c2)C3=O)N1 ; CC[C@@H]1CCC[C@H](c2cc3c(c(C(F)(F)F)c2)CN(c2cccc(C4(Cc5nncn5C)CC(F)(F)C4)c2)C3=O)N1]

A first intermediate, 6-(4,4,5,5-tetramethyl-1,3,2-dioxaborolan-2-yl)-4-(trifluoromethyl)isoindolin-1-one, can be synthesized as follows. A mixture of 6-bromo-4-(trifluoromethyl)isoindolin-1-one (2.0 g, 7.14 mmol), bis(pinacolato)diboron (2.2 g, 8.57 mmol), potassium acetate (2.1 g, 21.43 mmol) and 1,1′-bis(diphenylphosphino)ferrocene palladium dichloride (0.5 g, 0.71 mmol) in 1,4-dioxane (70 mL) was stirred at 80° C. for 3 h under nitrogen protection. The reaction was concentrated under reduce pressure and the residue was purified by silica gel chromatography (mobile phase: methanol/dichloro-methane, gradient 0% to 10%) to afford 6-(4,4,5,5-tetramethyl-1,3,2-dioxaborolan-2-yl)-4-(trifluoromethyl)isoindolin-1-one (2.3 g, 98% yield) as a brown solid. LCMS [M+H]⁺=328.2.

A second intermediate, 6-(6-ethylpyridin-2-yl)-4-(trifluoromethyl)isoindolin-1-one, can be synthesized as follows. A mixture of 6-(4,4,5,5-tetramethyl-1,3,2-dioxaborolan-2-yl)-4-(trifluoromethyl)isoindolin-1-one (2.5 g, 7.74 mmol), 2-bromo-6-ethyl-pyridine (1.2 g, 6.45 mmol), cesium carbonate (6.3 g, 19.35 mmol) and 1,1′-bis(diphenylphosphino)ferrocene palladium dichloride (471.9 mg, 0.64 mmol) in 1,4-dioxane (65 mL) was stirred at 90° C. for 16 h under nitrogen atmosphere. The reaction solution was concentrated under reduce pressure and the residue was purified by silica gel chromatography (mobile phase: methanol/dichloromethane, gradient 0% to 6%) to afford 6-(6-ethylpyridin-2-yl)-4-(trifluoro-methyl)isoindolin-1-one (1.7 g, 86% yield) as a white solid. LCMS [M+H]⁺=307.0.

A third intermediate, 6-(6-ethylpiperidin-2-yl)-4-(trifluoromethyl)isoindolin-1-one, can be synthesized as follows. To a solution of 6-(6-ethyl-2-pyridyl)-4-(trifluoromethyl)-isoindolin-1-one (700 mg, 2.29 mmol) in methanol (20 mL) and acetic acid (4 mL) was added Platinum(IV) oxide (52 mg, 0.23 mmol). Then the mixture was stirred at 25° C. for 16 h under H2 atmosphere (15 Psi). The reaction solution was filtered then the filtrate was concentrated to afford crude 6-(6-ethyl-2-piperidyl)-4-(trifluoromethyl)isoindolin-1-one (700 mg, 98% yield) as a white solid. LCMS [M+H]⁺=313.1.

A mixture of 3-((1-(3-bromophenyl)-3,3-difluorocyclobutyl)methyl)-4-methyl-4H-1,2,4-triazole (376 mg, 1.15 mmol), 6-(6-ethylpiperidin-2-yl)-4-(trifluoromethyl)isoindolin-1-one (300 mg, 0.96 mmol), cesium carbonate (939 mg, 2.88 mmol) and Xantphos Pd G3 (199 mg, 0.19 mmol) in 2-methyl-2-propanol (10 mL) was stirred at 100° C. for 16 h under nitrogen atmosphere. The mixture was concentrated under reduce pressure then the residue was purified by preparative TLC (solvent gradient: 10% methanol in dichloromethane) to afford a mixture of isomers, Compounds 273 and 274: 2-(3-(3,3-difluoro-1-((4-methyl-4H-1,2,4-triazol-3-yl)methyl)cyclobutyl)phenyl)-6-(6-ethylpiperidin-2-yl)-4-(trifluoromethyl)isoindolin-1-one (130 mg, 24% yield) as a white solid.

The above mixture was further purified by chiral SFC (Column=Daicel Chiralcel OD-H; Column dimensions=250 mm×30 mm×5 μm; Detection wavelength=220 nM; Flow rate=80 mL/min; Mobile phase: A: CO₂ B: ethanol (0.05% DEA); gradient 5% to 40%; Column temperature: 40° C.) with 0.1% ammonium hydroxide) to afford tentatively assigned:

2-(3-(3,3-difluoro-1-((4-methyl-4H-1,2,4-triazol-3-yl)methyl)cyclobutyl)phenyl)-6-((2S,6S)-6-ethylpiperidin-2-yl)-4-(trifluoromethyl)isoindolin-1-one (Peak 1, retention time=4.641 min) (44 mg, 32% yield). ¹H NMR (400 MHz, methanol-d₄) δ 8.16 (s, 1H), 8.10 (s, 1H), 8.03 (s, 1H), 7.73 (dd, J=1.6, 8.0 Hz, 1H), 7.49-7.47 (m, 1H), 7.38 (t, J=8.0 Hz, 1H), 6.81 (d, J=8.0 Hz, 1H), 5.06 (s, 2H), 3.92-3.87 (m, 1H), 3.35 (s, 2H), 3.28-3.22 (m, 2H), 3.10-2.97 (m, 2H), 2.76 (s, 3H), 2.67-2.58 (m, 1H), 1.98-1.91 (m, 1H), 1.88-1.79 (m, 2H), 1.63-1.44 (m, 4H), 1.21-1.09 (m, 1H), 0.96 (t, J=7.6 Hz, 3H) LCMS: [M+H]⁺=574.3.

2-(3-(3,3-difluoro-1-((4-methyl-4H-1,2,4-triazol-3-yl)methyl)cyclobutyl)phenyl)-6-((2R,6R)-6-ethylpiperidin-2-yl)-4-(trifluoromethyl)isoindolin-1-one (Peak 2, retention time=4.845 min) (51 mg, 37% yield). ¹H NMR (400 MHz, methanol-d₄) δ 8.16 (s, 1H), 8.10 (s, 1H), 8.03 (s, 1H), 7.73 (dd, J=1.6, 8.0 Hz, 1H), 7.49-7.47 (m, 1H), 7.38 (t, J=8.0 Hz, 1H), 6.81 (d, J=8.0 Hz, 1H), 5.06 (s, 2H), 3.92-3.87 (m, 1H), 3.35 (s, 2H), 3.28-3.22 (m, 2H), 3.08-2.97 (m, 2H), 2.76 (s, 3H), 2.68-2.59 (m, 1H), 1.99-1.91 (m, 1H), 1.88-1.79 (m, 2H), 1.63-1.45 (m, 4H), 1.21-1.10 (m, 1H), 0.96 (t, J=7.6 Hz, 3H). LCMS: [M+H]⁺=574.3.

### Example 75: Quantitative Data for Further Isoindolin-1-one Compounds

Additional isoindolin-1-one compounds can be synthesized according to the methods herein. Quantitative data for examples of such compounds is as follows.

TABLE 2
Quantitative Analytical Data on Additional Isoindolin-1-one Compounds

| Compound | | LCMS |
|---|---|---|
| # | ¹H NMR | [M + H]⁺ or [M − H]⁻ |
| 66 | ¹H NMR (400 MHz, methanol-d4): δ 8.22-8.09 (m, 3H), 7.76 | 458.1 |
| | (d, J = 8.4 Hz, 1H), 7.51-7.31 (m, 3H), 6.81 (d, J = 8.0 Hz, | |
| | 1H), 5.15 (s, 2H), 5.10-5.06 (m, 4H), 4.33-4.32 (m, 2H), | |
| | 3.67 (s, 2H), 2.92 (s, 3H). | |
| 68 | ¹H NMR (400 MHz, methanol-d4): δ 8.28 (s, 1H), 8.09 (s, 1H), | 476.2 |
| | 8.02 (s, 1H), 7.80 (dd, J = 1.6, 8.0 Hz, 1H), 7.53 (t, J = 1.6 Hz, | |
| | 1H), 7.43 (t, J = 7.6 Hz, 1H), 6.93 (d, J = 8.0 Hz, 1H), 6.31 (d, | |
| | J = 45.2 Hz, 1H), 5.48 (d, J = 6.4 Hz, 1H), 5.32 (d, J = 6.4 Hz, | |
| | 1H), 5.22 (dd, J = 1.6, 6.4 Hz, 1H), 5.11 (br s, 2H), 5.03-5.01 | |
| | (dd, J = 3.6, 6.0 Hz, 1H), 4.09 (s, 2H), 3.12 (s, 3H) | |
| 69 | ¹H NMR (400 MHz, methanol-d4): δ 8.28 (s, 1H), 8.07 (s, 1H), | 476.1 |
| | 7.99 (s, 1H), 7.80-7.78 (dd, J = 1.6, 8.0 Hz, 1H), 7.53 (t, J = | |
| | 1.6 Hz, 1H), 7.43 (t, J = 8.0 Hz, 1H), 6.92 (d, J = 8.0 Hz, 1H), | |
| | 6.31 (d, J = 45.6 Hz, 1H), 5.48 (d, J = 6.4 Hz, 1H), 5.32 (d, J = | |
| | 6.4 Hz, 1H), 5.22 (dd, J = 1.6, 6.4 Hz, 1H), 5.09 (br s, 2H), | |
| | 5.02-5.00 (dd, J = 4.4, 6.4 Hz, 1H), 4.01 (s, 2H), 3.12 (s, 3H) | |
| 70 | ¹H NMR (400 MHz, DMSO-d6): δ 8.35 (s, 1H), 7.93-7.83 (m, | 516.1 |
| | 3H), 7.55 (s, 1H), 7.37 (t, J = 8.0 Hz, 1H), 6.96 (d, J = 7.6 Hz, | |
| | 1H), 6.27 (d, J = 45.6 Hz, 1H), 5.36 (d, J = 6.8 Hz, 1H), 5.21 | |
| | (d, J = 7.6 Hz, 1H), 5.14-5.02 (m, 3H), 4.82 (dd, J = 4.0, 6.0 | |
| | Hz, 1H), 3.72 (s, 2H), 3.21-3.11 (m, 7H), 2.07-1.97 (m, 2H) | |
| 71 | ¹H NMR (400 MHz, methanol-d4): δ 8.28 (s, 1H), 8.03 (s, 1H), | 516.1 |
| | 7.94 (s, 1H), 7.79 (dd, J = 1.2, 8.0 Hz, 1H), 7.52 (t, J = 8.0 Hz, | |
| | 1H), 7.44 (t, J = 8.0 Hz, 1H), 6.94 (d, J = 7.6 Hz, 1H), 6.31 (d, | |
| | J = 45.2 Hz, 1H), 5.48 (d, J = 6.4 Hz, 1H), 5.32 (d, J = 6.0 Hz, | |
| | 1H), 5.22 (dd, J = 2.0, 6.4 Hz, 1H), 5.10 (br s, 2H), 5.02 (dd, | |
| | J = 4.4, 6.4 Hz, 1H), 3.93 (s, 2H), 3.48 (t, J = 7.2 Hz, 1H), 3.12 | |
| | (s, 3H), 2.25-2.18 (m, 2H) | |
| 72 | ¹H NMR (400 MHz, DMSO-d6): δ 8.35 (s, 1H), 7.95-7.90 (m, | 544.1 |
| | 2H), 7.89 (s, 1H), 7.56 (s, 1H), 7.37 (t, J = 8.0 Hz, 1H), 6.97 | |
| | (d, J = 8.0 Hz, 1H), 6.27 (d, J = 46 Hz, 1H), 5.36 (d, J = 6.8 | |
| | Hz, 1H), 5.21 (d, J = 6.0 Hz, 1H), 5.16-5.05 (m, 3H), 4.83 | |
| | (dd, J = 4.0, 6.0 Hz, 1H), 3.76 (s, 2H), 3.18 (s, 3H), 2.94 (s, | |
| | 4H), 1.20 (s, 6H) | |
| 73 | ¹H NMR (400 MHz, DMSO-d6): δ 8.35 (s, 1H), 7.94-7.87 (m, | 544.1 |
| | 3H), 7.56 (s, 1H), 7.37 (t, J = 8.0 Hz, 1H), 6.97 (d, J = 8.0 Hz, | |
| | 1H), 6.27 (d, J = 46 Hz, 1H), 5.36 (d, J = 6.8 Hz, 1H), 5.21 (d, | |
| | J = 6.0 Hz, 1H), 5.15-5.04 (m, 3H), 4.83 (dd, J = 4.4, 6.0 Hz, | |
| | 1H), 3.76 (s, 2H), 3.18 (s, 3H), 2.94 (s, 4H), 1.20 (s, 6H) | |
| 74 | ¹H NMR (400 MHz, DMSO-d6): δ 8.35 (s, 1H), 7.93-7.90 (m, | 560.1 |
| | 2H), 7.88 (s, 1H), 7.56 (s, 1H), 7.38 (t, J = 8.0 Hz, 1H), 6.97 | |
| | (d, J = 7.6 Hz, 1H), 6.27 (d, J = 46 Hz, 1H), 5.37 (d, J = 6.0 | |
| | Hz, 1H), 5.21 (d, J = 6.0 Hz, 1H), 5.15-5.07 (m, 3H), 4.83 | |
| | (dd, J = 4.0, 6.0 Hz, 1H), 4.74-4.67 (m, 1H), 3.76 (s, 2H), | |
| | 3.39 (d, J = 5.6 Hz, 2H), 3.18 (s, 3H), 3.09 (d, J = 6.8 Hz, 2H), | |
| | 2.82 (d, J = 6.8 Hz, 2H), 1.16 (s, 3H) | |
| 75 | ¹H NMR (400 MHz, DMSO-d6): δ 8.35 (s, 1H), 7.93-7.90 (m, | 560.1 |
| | 2H), 7.88 (s, 1H), 7.56 (s, 1H), 7.38 (t, J = 8.0 Hz, 1H), 6.97 | |
| | (d, J = 7.6 Hz, 1H), 6.27 (d, J = 45.6 Hz, 1H), 5.37 (d, J = 6.4 | |
| | Hz, 1H), 5.21 (d, J = 6.0 Hz, 1H), 5.14-5.06 (m, 3H), 4.85- | |
| | 4.80 (m, 1H), 4.76-4.70 (m, 1H), 3.76 (s, 2H), 3.39 (d, J = | |
| | 5.2 Hz, 2H), 3.18 (s, 3H), 3.09 (d, J = 6.8 Hz, 2H), 2.82 (d, J = | |
| | 6.8 Hz, 2H), 1.16 (s, 3H) | |
| 76 | ¹H NMR (400 MHz, CDCl3): δ 8.00 (s, 1H), 7.91 (s, 1H), 7.80 | 562.1 |
| | (s, 1H), 7.64 (dd, J = 1.6, 8.0 Hz, 1H), 7.47 (s, 1H), 7.37 (t, J = | |
| | 8.0 Hz, 1H), 6.80-6.74 (d, J = 8.0 Hz, 1H), 6.47 (d, J = 46.0 | |
| | Hz, 1H), 5.38-5.21 (m, 3H), 5.02-4.84 (m, 3H), 4.39 (d, J = | |
| | 47.6 Hz, 2H), 3.78 (s, 2H), 3.25 (d, J = 7.2 Hz, 2H), 3.03 (s, | |
| | 3H), 2.99 (dd, J = 2.4, 7.6 Hz, 1H), 1.31 (s, 3H) | |
| 77 | ¹H NMR (400 MHz, CDCl3): δ 8.00 (s, 1H), 7.91 (s, 1H), 7.81 | 562.1 |
| | (s, 1H), 7.64 (dd, J = 1.6, 8.0 Hz, 1H), 7.47 (s, 1H), 7.37 (t, J = | |
| | 8.0 Hz, 1H), 6.77 (d, J = 7.2 Hz, 1H), 6.47 (d, J = 46.4 Hz, | |
| | 1H), 5.35-5.21 (m, 3H), 5.02-4.85 (m, 3H), 4.40 (d, J = 47.6 | |
| | Hz, 2H), 3.78 (s, 2H), 3.26 (d, J = 7.2 Hz, 2H), 3.04 (s, 3H), | |
| | 3.02-2.94 (m, 2H), 1.31 (s, 3H) | |
| 78 | ¹H NMR (400 MHz, CDCl3): δ 8.02 (s, 1H), 7.91 (s, 2H), 7.64 | 542.1 |
| | (dd, J = 1.2, 8.0 Hz, 1H), 7.46 (s, 1H), 7.37 (t, J = 8.0 Hz, 1H), | |
| | 6.77 (d, J = 7.6 Hz, 1H), 6.46 (d, J = 46.0 Hz, 1H), 5.35-5.21 | |
| | (m, 3H), 5.02-4.86 (m, 3H), 3.93 (s, 2H), 3.47 (s, 4H), 3.03 | |
| | (d, J = 1.2 Hz, 3H), 0.60 (s, 4H) | |
| 79 | ¹H NMR (400 MHz, CDCl3): δ 8.02 (s, 1H), 7.91 (s, 1H), 7.89 | 542.1 |
| | (s, 1H), 7.65 (dd, J = 1.2, 8.0 Hz, 1H), 7.46 (br s, 1H), 7.37 (t, | |
| | J = 8.0 Hz, 1H), 6.77 (d, J = 7.6 Hz, 1H), 6.46 (d, J = 46.0 Hz, | |
| | 1H), 5.34-5.21 (m, 3H), 5.02-4.85 (m, 3H), 3.92 (s, 2H), | |
| | 3.46 (s, 4H), 3.03 (d, J = 1.2 Hz, 3H), 0.60 (s, 4H) | |
| 80 | ¹H NMR (400 MHz, DMSO-d6): δ 8.35 (s, 1H), 8.00 (s, 1H), | 548.1 |
| | 7.97-7.91 (m, 2H), 7.56 (s, 1H), 7.38 (t, J = 8.0 Hz, 1H), 6.97 | |
| | (d, J = 7.6 Hz, 1H), 6.27 (d, J = 46 Hz, 1H), 5.37 (d, J = 6.8 | |
| | Hz, 1H), 5.29 (t, J = 5.6 Hz, 1H), 5.22 (d, J = 6.0 Hz, 1H), 5.16 | |
| | (d, J = 6.4 Hz, 1H), 5.14-5.09 (m, 3H), 4.83 (dd, J = 4.0, 6.0 Hz, | |
| | 1H), 3.84 (s, 2H), 3.18 (s, 3H), 2.87-2.75 (m, 2H), 2.73-2.57 (m, | |
| | 1H), 2.41-2.32 (m, 1H), 2.26-2.09 (m, 1H), 2.03-1.78 (m, 1H) | |
| 81 | ¹H NMR (400 MHz, DMSO-d6): δ 8.35 (s, 1H), 8.00 (s, 1H), | 548.1 |
| | 7.97-7.91 (m, 2H), 7.55 (s, 1H), 7.38 (t, J = 8.0 Hz, 1H), 6.97 | |
| | (d, J = 7.6 Hz, 1H), 6.27 (d, J = 46 Hz, 1H), 5.37 (d, J = 6.8 | |
| | Hz, 1H), 5.29 (t, J = 6.4 Hz, 1H), 5.22 (d, J = 6.0 Hz, 1H), 5.16 | |
| | (d, J = 6.4 Hz, 1H), 5.14-5.09 (m, 3H), 4.83 (dd, J = 4.0, 6.0 Hz, | |
| | 1H), 3.84 (s, 2H), 3.18 (s, 3H), 2.87-2.75 (m, 2H), 2.73-2.57 (m, | |
| | 1H), 2.41-2.32 (m, 1H), 2.26-2.09 (m, 1H), 2.03-1.78 (m, 1H) | |
| 82 | ¹H NMR (400 MHz, CDCl3): δ 8.05 (s, 1H), 7.92-7.87 (m, 2H), | 548.1 |
| | 7.66 (dd, J = 2.0, 8.0 Hz, 1H), 7.45 (s, 1H), 7.38 (t, J = 8.0 Hz, | |
| | 1H), 6.78 (d, J = 8.0 Hz, 1H), 6.47 (d, J = 46 Hz, 1H), 5.34-5.13 | |
| | (m, 4H), 5.02-4.88 (m, 3H), 3.83 (s, 2H), 3.04 (d, J = 1.2 Hz, | |
| | 3H), 2.93-2.77 (m, 3H), 2.57-2.47 (m, 1H), 2.27-2.02 (m, 2H) | |
| 83 | ¹H NMR (400 MHz, CDCl3): δ 7.97 (br s, 1H), 7.83 (s 1H), | 548.1 |
| | 7.81 (br s, 1H), 7.58 (dd, J = 1.2, 8.0 Hz, 1H), 7.37 (s, 1H), | |
| | 7.30 (t, J = 8.0 Hz, 1H), 6.71 (d, J = 8.0 Hz, 1H), 6.39 (d, J = | |
| | 46 Hz, 1H), 5.25-5.04 (m, 4H), 4.93-4.79 (m, 3H), 3.76 (d, | |
| | J = 2.0 Hz, 2H), 2.96 (d, J = 1.6 Hz, 3H), 2.83-2.69 (m, 3H), | |
| | 2.48-2.40 (m, 1H), 2.19-1.94 (m, 2H) | |
| 84 | ¹H NMR (400 MHz, DMSO-d6): δ 8.35 (s, 1H), 7.96-7.89 (m, | 542.1 |
| | 2H), 7.87 (s, 1H), 7.56 (br s, 1H), 7.38 (t, J = 8.0 Hz, 1H), 6.97 | |
| | (d, J = 7.6 Hz, 1H), 6.27 (d, J = 46 Hz, 1H), 5.36 (d, J = 6.8 | |
| | Hz, 1H), 5.21 (d, J = 6.0 Hz, 1H), 5.15-5.05 (m, 3H), 4.83 | |
| | (dd, J = 4.0, 6.0 Hz, 1H), 3.78 (s, 2H), 3.18 (s, 3H), 2.88 (d, J = | |
| | 8.8 Hz, 2H), 2.37 (d, J = 8.4 Hz, 2H), 1.42-1.33 (m, 2H), | |
| | 0.69-0.66 (m, 1H), 0.37-0.33 (m, 1H) | |
| 85 | ¹H NMR (400 MHz, DMSO-d6): δ 8.35 (s, 1H), 7.99-7.82 (m, | 542.1 |
| | 3H), 7.56 (br s, 1H), 7.38 (t, J = 8.0 Hz, 1H), 6.97 (d, J = 7.2 | |
| | Hz, 1H), 6.27 (d, J = 45.6 Hz, 1H), 5.36 (d, J = 6.0 Hz, 1H), | |
| | 5.21 (d, J = 5.2 Hz, 1H), 5.17-5.04 (m, 3H), 4.83 (br s, 1H), | |
| | 3.79 (br s, 2H), 3.18 (s, 3H), 2.97-2.80 (m, 2H), 2.37 (br s, | |
| | 2H), 1.39 (br s, 2H), 0.69 (br s, 1H), 0.36 (br s, 1H) | |
| 86 | ¹H NMR (400 MHz, CDCl3): δ 8.10 (s, 1H), 8.01 (s, 1H), 7.91 | 542.1 |
| | (s, 1H), 7.65 (dd, J = 1.6, 8.4 Hz, 1H), 7.47 (br s, 1H), 7.37 (t, | |
| | J = 8.0 Hz, 1H), 6.77 (d, J = 8.0 Hz, 1H), 6.46 (d, J = 46 Hz, | |
| | 1H), 5.34-5.21 (m, 3H), 5.03-4.85 (m, 3H), 3.95 (s, 2H), | |
| | 3.50-3.44 (m, 1H), 3.03 (d, J = 1.6 Hz, 1H), 2.83-2.77 (m, 1H), | |
| | 2.75 (s, 2H), 1.80-1.72 (m, 2H), 1.58 (dd, J = 2.0, 4.8 Hz, 2H) | |
| 87 | ¹H NMR (400 MHz, CDCl3): δ 8.10 (s, 1H), 7.98 (s, 1H), 7.91 | 542.1 |
| | (s, 1H), 7.65 (dd, J = 1.2, 8.0 Hz, 1H), 7.45 (br s, 1H), 7.37 (t, | |
| | J = 8.0 Hz, 1H), 6.76 (d, J = 8.0 Hz, 1H), 6.46 (d, J = 45.6 Hz, | |
| | 1H), 5.34-5.21 (m, 3H), 5.03-4.85 (m, 3H), 3.93 (s, 2H), | |
| | 3.48-3.42 (m, 1H), 3.03 (d, J = 1.2 Hz, 3H), 2.83-2.77 (m, 1H), | |
| | 2.72 (s, 2H), 1.80-1.72 (m, 2H), 1.56 (dd, J = 2.0, 4.8 Hz, 2H) | |
| 88 | ¹H NMR (400 MHz, methanol-d4): δ 8.28 (s, 1H), 8.07 (s, 1H), | 546.1 |
| | 7.97 (s, 1H), 7.80 (dd, J = 1.6, 8.0 Hz, 1H), 7.52 (s, 1H), 7.43 | |
| | (t, J = 8.0 Hz, 1H), 6.93 (d, J = 8.4 Hz, 1H), 6.31 (d, J = 45.6 | |
| | Hz, 1H), 5.48 (d, J = 6.8 Hz, 1H), 5.31 (d, J = 6.4 Hz, 1H), | |
| | 5.21 (dd, J = 1.6, 6.4 Hz, 1H), 5.09 (br s, 2H), 5.02 (dd, J = 4.0, | |
| | 6.0 Hz, 1H), 3.77-3.67 (m, 6H), 3.12 (s, 3H), 2.55-2.48 (m, 4H) | |
| 89 | ¹H NMR (400 MHz, methanol-d4): δ 8.28 (s, 1H), 8.07 (s, 1H), | 546.1 |
| | 7.97 (s, 1H), 7.80 (dd, J = 1.6, 6.4 Hz, 1H), 7.52 (s, 1H), 7.43 | |
| | (t, J = 8.0 Hz, 1H), 6.93 (d, J = 7.6 Hz, 1H), 6.31 (d, J = 45.2 | |
| | Hz, 1H), 5.48 (d, J = 6.8 Hz, 1H), 5.31 (d, J = 6.0 Hz, 1H), | |
| | 5.22 (dd, J = 1.6, 6.4 Hz, 1H), 5.10 (br s, 2H), 5.02 (dd, J = 4.0, | |
| | 6.0 Hz, 1H), 3.77-3.67 (m, 6H), 3.12 (s, 3H), 2.55-2.47 (m, 4H). | |
| 90 | ¹H NMR (400 MHz, methanol-d4): δ 8.28 (s, 1H), 8.06 (s, 1H), | 587.3 |
| | 7.97 (s, 1H), 7.79 (dd, J = 1.6, 8.0 Hz, 1H), 7.52 (t, J = 1.6 Hz, | |
| | 1H), 7.43 (t, J = 8.0 Hz, 1H), 6.93 (d, J = 8.0 Hz, 1H), 6.30 (d, | |
| | J = 45.2 Hz, 1H), 5.48 (d, J = 6.4 Hz, 1H), 5.31 (d, J = 6.4 Hz, | |
| | 1H), 5.21 (dd, J = 1.6, 6.4 Hz, 1H), 5.09 (s, 2H), 5.02 (dd, J = | |
| | 4.0, 6.4 Hz, 1H), 3.78-3.68 (m, 2H), 3.11 (s, 3 H), 3.00-2.89 | |
| | (m, 2H), 2.84 (td, J = 2.8, 12 Hz, 1H), 2.76 (d, J = 10.8 Hz, | |
| | 1H), 2.53-2.44 (m, 1H), 2.15 (td, J = 3.2, 11.2 Hz, 1H), 1.90 | |
| | (t, J = 10.8 Hz, 1H), 1.66-1.53 (m, 1H), 0.96 (d, J = 6.8 Hz, | |
| | 3H), 0.89 (d, J = 6.8 Hz, 3H). | |
| 91 | ¹H NMR (400 MHz, methanol-d4): δ 8.27 (s, 1H), 8.05 (s, 1H), | 587.3 |
| | 7.96 (s, 1H), 7.79 (dd, J = 1.6, 8.4 Hz, 1H), 7.51 (t, J = 1.6 Hz, | |
| | 1H), 7.42 (t, J = 8.0 Hz, 1H), 6.92 (d, J = 7.6 Hz, 1H), 6.30 (d, | |
| | J = 45.2 Hz, 1H), 5.47 (d, J = 6.4 Hz, 1H), 5.31 (d, J = 6.4 Hz, | |
| | 1H), 5.20 (dd, J = 2.0, 6.8 Hz, 1H), 5.09 (s, 2H), 5.01 (dd, J = | |
| | 4.4, 6.4 Hz, 1H), 3.77-3.68 (m, 2H), 3.11 (s, 3H), 3.01-2.95 | |
| | (m, 1H), 2.92 (d, J = 10.8 Hz, 1H), 2.85 (td, J = 3.2, 12.0 Hz, | |
| | 1H), 2.76 (d, J = 10.8 Hz, 1H), 2.53-2.46 (m, 1H), 2.15 (td, J = | |
| | 3.2, 11.2 Hz, 1H), 1.93-1.87 (m, 1H), 1.65-1.55 (m, 1H), | |
| | 0.96 (d, J = 6.8 Hz, 3H), 0.89 (d, J = 6.8 Hz, 3H). | |
| 92 | ¹H NMR (400 MHz, methanol-d4): δ 8.27 (s, 1H), 8.05 (s, 1H), | 587.3 |
| | 7.96 (s, 1H), 7.78 (dd, J = 1.6, 8.0 Hz, 1H), 7.51 (t, J = 2.0 Hz, | |
| | 1H), 7.42 (t, J = 8.0 Hz, 1H), 6.92 (d, J = 7.6 Hz, 1H), 6.29 (d, | |
| | J = 45.2 Hz, 1H), 5.47 (d, J = 6.4 Hz, 1H), 5.31 (d, J = 6.4 Hz, | |
| | 1H), 5.20 (dd, J = 1.6, 6.4 Hz, 1H), 5.09 (s, 2H), 5.01 (dd, J = | |
| | 4.0, 6.4 Hz, 1H), 3.67-3.78 (m, 2H), 3.10 (s, 3H), 2.99-2.88 | |
| | (m, 2H), 2.83 (td, J = 2.8, 11.6 Hz, 1H), 2.75 (d, J = 12.0 Hz, | |
| | 1H), 2.52-2.44 (m, 1H), 2.14 (td, J = 2.8, 11.2 Hz, 1H), 1.89 | |
| | (t, J = 10.8 Hz, 1H), 1.64-1.53 (m, 1H), 0.95 (d, J = 6.8 Hz, | |
| | 3H), 0.88 (d, J = 6.8 Hz, 3H). | |
| 93 | ¹H NMR (400 MHz, methanol-d4): δ 8.28 (s, 1H), 8.06 (s, 1H), | 587.4 |
| | 7.97 (s, 1H), 7.79 (dd, J = 1.6, 8.0 Hz, 1H), 7.52 (t, J = 1.6 Hz, | |
| | 1H), 7.43 (t, J = 8.0 Hz, 1H), 6.93 (d, J = 8.0 Hz, 1H), 6.30 (d, | |
| | J = 45.6 Hz, 1H), 5.48 (d, J = 6.4 Hz, 1H), 5.31 (d, J = 6.4 Hz, | |
| | 1H), 5.21 (dd, J = 1.6, 6.8 Hz, 1H), 5.10 (s, 2H), 5.02 (dd, J = | |
| | 4.0, 6.4 Hz, 1H), 3.78-3.68 (m, 2H), 3.11 (s, 3 H), 3.02-2.96 | |
| | (m, 1H), 2.92 (d, J = 6.4 Hz, 1H), 2.86 (td, J = 3.2, 11.6 Hz, | |
| | 1H), 2.77 (d, J = 10.8 Hz, 1H), 2.56-2.47 (m, 1H), 2.16 (td, J = | |
| | 3.2, 11.2 Hz, 1H), 1.91 (t, J = 10.8 Hz, 1H), 1.66-1.56 (m, | |
| | 1H), 0.96 (d, J = 6.8 Hz, 3H), 0.90 (d, J = 6.8 Hz, 3H). | |
| 94 | ¹H NMR (400 MHz, methanol-d4): δ 8.28 (s, 1H), 8.08 (s, 1H), | 587.2 |
| | 7.94 (s, 1H), 7.79 (dd, J = 1.6, 8.0 Hz, 1H), 7.51 (s, 1H), 7.43 | |
| | (t, J = 8.0 Hz, 1H), 6.93 (d, J = 8.0 Hz, 1H), 6.30 (d, J = 45.2 | |
| | Hz, 1H), 5.48 (d, J = 6.4 Hz, 1H), 5.31 (d, J = 6.4 Hz, 1H), | |
| | 5.21 (dd, J = 1.6, 6.4 Hz, 1H), 5.08 (br s, 2H), 5.01 (dd, J = | |
| | 4.4, 6.4 Hz, 1H), 4.35 (d, J = 14.4 Hz, 1H), 3.36-3.32 (m, | |
| | 1H), 3.11 (s, 3H), 2.96 (d, J = 10.8 Hz, 1H), 2.85-2.78 (m, | |
| | 1H), 2.75-2.67 (m, 2H), 2.63 (dd, J = 10.0, 12.4 Hz, 1H), 2.39- | |
| | 2.31 (m, 1H), 2.28-2.17 (m, 2H), 1.06-0.93 (m, 6H). | |
| 95 | ¹H NMR (400 MHz, methanol-d4): δ 8.28 (s, 1H), 8.10 (s, 1H), | 587.3 |
| | 7.95 (s, 1H), 7.79 (dd, J = 1.6, 8.0 Hz, 1H), 7.51 (t, J = 1.6 Hz, | |
| | 1H), 7.43 (t, J = 8.0 Hz, 1H), 6.94 (d, J = 8.0 Hz, 1H), 6.30 (d, | |
| | J = 45.2 Hz, 1H), 5.48 (d, J = 6.4 Hz, 1H), 5.31 (d, J = 6.4 Hz, | |
| | 1H), 5.21 (dd, J = 1.6, 6.4 Hz, 1H), 5.10 (s, 2H), 5.02 (dd, J = | |
| | 4.0, 6.4 Hz, 1H), 4.37 (d, J = 14.4 Hz, 1H), 3.48 (d, J = 14.4 | |
| | Hz, 1H), 3.37 (d, J = 12.4 Hz, 1H), 3.25 (d, J = 12.4 Hz, 1H), | |
| | 3.11 (s, 3H), 3.09-2.90 (m, 3H), 2.65-2.55 (m, 1H), 2.55- | |
| | 2.37 (m, 2H), 1.07-0.97 (m, 6H). | |
| 96 | ¹H NMR (400 MHz, methanol-d4): δ 8.28 (s, 1H), 8.08 (s, 1H), | 587.2 |
| | 7.94 (s, 1H), 7.79 (dd, J = 1.2, 8.0 Hz, 1H), 7.51 (s, 1H), 7.43 | |
| | (t, J = 8.0 Hz, 1H), 6.93 (d, J = 8.0 Hz, 1H), 6.30 (d, J = 45.6 | |
| | Hz, 1H), 5.48 (d, J = 6.8 Hz, 1H), 5.31 (d, J = 6.4 Hz, 1H), | |
| | 5.21 (d, J = 6.8 Hz, 1H), 5.09 (s, 2H), 5.01 (dd, J = 4.4, 6.4 Hz, | |
| | 1H), 4.35 (d, J = 14.4 Hz, 1H), 3.36 (s, 1H), 3.11 (s, 3H), 2.99 | |
| | (d, J = 11.2 Hz, 1H), 2.87-2.81 (m, 1H), 2.77-2.63 (m, 3H), 2.36 | |
| | (dd, J = 6.4, 12.4 Hz, 1H), 2.31-2.19 (m, 2H), 1.05-0.94 (m, 6H). | |
| 97 | ¹H NMR (400 MHz, methanol-d4): δ 8.28 (s, 1H), 8.11 (s, 1H), | 587.2 |
| | 7.96 (s, 1H), 7.79 (dd, J = 1.6, 8.4 Hz, 1H), 7.52 (s, 1H), 7.45 | |
| | (t, J = 8.0 Hz, 1H), 6.97 (d, J = 8.0 Hz, 1H), 6.31 (d, J = 45.2 | |
| | Hz, 1H), 5.48 (d, J = 6.4 Hz, 1H), 5.32 (d, J = 6.4 Hz, 1H), | |
| | 5.22 (dd, J = 1.6, 6.4 Hz, 1H), 5.11 (d, J = 4.0 Hz, 2H), 5.02 | |
| | (dd, J = 4.0, 6.4 Hz, 1H), 4.39 (d, J = 14.4 Hz, 1H), 3.48 (d, J = | |
| | 14.4 Hz, 1H), 3.37 (d, J = 11.6 Hz, 1H), 3.25 (d, J = 12.4 Hz, | |
| | 1H), 3.13 (s, 3H), 3.11-2.91 (m, 3H), 2.61-2.56 (m, 1H), 2.54- | |
| | 2.40 (m, 2H), 1.06 (d, J = 6.8 Hz, 3H), 1.01 (d, J = 6.8 Hz, 3H). | |
| 99 | ¹H NMR (400 MHz, methanol-d4): δ 8.18 (s, 1H), 8.09 (d, J = | 445.1 |
| | 7.6 Hz, 1H), 7.98 (d, J = 7.6 Hz, 1H), 7.78 (t, J = 7.6 Hz, 1H), | |
| | 7.71 (dd, J = 1.6, 8.0 Hz, 1H), 7.43 (t, J = 1.6 Hz, 1H), 7.38 | |
| | (t, J = 8.0 Hz, 1H), 6.79 (d, J = 8.4 Hz, 1H), 5.48-5.24 (m, | |
| | 1H), 5.11 (s, 2H), 3.22 (s, 2H), 3.20-3.11 (m, 2H), 2.76 (s, | |
| | 3H), 2.63-2.42 (m, 2H). | |
| 100 | ¹H NMR (400 MHz, methanol-d4): δ 8.17 (s, 1H), 8.09 (d, J = | 445.0 |
| | 7.6 Hz, 1H), 7.98 (d, J = 8.0 Hz, 1H), 7.78 (t, J = 8.0 Hz, 1H), | |
| | 7.71 (dd, J = 1.2, 8.0 Hz, 1H), 7.58 (t, J = 2.0 Hz, 1H), 7.39 (t, | |
| | J = 8.0 Hz, 1H), 6.95 (d, J = 8.4 Hz, 1H), 5.11 (s, 2H), 5.03- | |
| | 4.97 (m, 1H), 3.37 (s, 2H), 3.06-2.94 (m, 2H), 2.85 (s, 3H), | |
| | 2.83-2.74 (m, 2H). | |
| 101 | ¹H NMR (400 MHz, CDCl3): δ 8.04 (s, 1H), 7.87 (s, 2H), 7.55- | 560.1 |
| | 7.48 (m, 2H), 7.33 (t, J = 8.0 Hz, 1H), 6.62 (d, J = 7.6 Hz, | |
| | 1H), 5.15 (d, J = 6.0 Hz, 2H), 5.08 (d, J = 6.0 Hz, 2H), 4.93 | |
| | (s, 2H), 3.77 (s, 2H), 3.60 (s, 2H), 3.53 (d, J = 7.2 Hz, 2H), | |
| | 2.87 (s, 3H), 2.77-2.70 (m, 1H), 2.66-2.55 (m, 3H), 2.59- | |
| | 2.43 (m, 1H), 2.16-2.00 (m, 1H), 1.58-1.52 (m, 1H). | |
| 102 | ¹H NMR (400 MHz, CDCl3): δ 8.04 (s, 1H), 7.89-7.85 (m, | 560.1 |
| | 2H), 7.56-7.48 (m, 2H), 7.33 (t, J = 8.0 Hz, 1H), 6.62 (d, J = | |
| | 8.0 Hz, 1H), 5.15 (d, J = 6.4 Hz, 2H), 5.08 (d, J = 6.4 Hz, 2H), | |
| | 4.93 (s, 2H), 3.77 (s, 2H), 3.60 (s, 2H), 3.53 (d, J = 6.8 Hz, | |
| | 2H), 2.87 (s, 3H), 2.77-2.70 (m, 1H), 2.66-2.56 (m, 3H), | |
| | 2.46 (s, 1H), 2.15-2.00 (m, 1H), 1.59-1.54 (m, 1H). | |
| 103 | ¹H NMR (400 MHz, CDCl3): δ 8.00 (s, 1H), 7.90 (s, 1H), 7.80 | 578.1 |
| | (s, 1H), 7.65 (d, J = 8.0 Hz, 2H), 7.47 (s, 1H), 7.37 (t, J = 7.6 | |
| | Hz, 1H), 6.77 (d, J = 7.6 Hz, 1H), 6.47 (d, J = 46.0 Hz, 1H), | |
| | 5.36-5.20 (m, 3H), 5.03-4.84 (m, 3H), 3.78 (s, 2H), 3.72 (s, | |
| | 2H), 3.22 (d, J = 7.2 Hz, 2H), 3.04 (s, 3H), 2.99 (d, J = 7.2 Hz, | |
| | 2H), 1.36 (s, 3H). | |
| 104 | ¹H NMR (400 MHz, CDCl3): δ 8.00 (s, 1H), 7.91 (s, 1H), 7.81 | 578.1 |
| | (s, 1H), 7.64 (dd, J = 1.2, 8.0 Hz, 1H), 7.47 (br s, 1H), 7.37 (t, | |
| | J = 8.0 Hz, 1H), 6.77 (d, J = 7.6 Hz, 1H), 6.46 (d, J = 46.0 Hz, | |
| | 1H), 5.34-5.21 (m, 3H), 5.02-4.85 (m, 3H), 3.81 (s, 2H), | |
| | 3.71 (s, 2H), 3.30-3.21 (m, 2H), 3.10-2.98 (m, 5H), 1.37 (s, 3H). | |
| 105 | ¹H NMR (400 MHz, methanol-d4): δ 8.18 (s, 1H), 8.15 (s, 1H), | 584.3 |
| | 8.06 (s, 1H), 7.75 (dd, J = 1.2, 8.0 Hz, 1H), 7.50 (br s, 1H), | |
| | 7.40 (d, J = 7.6 Hz, 1H), 7.35 (s, 1H), 6.84 (d, J = 8.0 Hz, 1H), | |
| | 5.11 (s, 2H), 4.19 (s, 2H), 3.84 (s, 4H), 3.37 (s, 2H), 3.27- | |
| | 3.26 (m, 2H), 3.12-2.96 (m, 2H), 2.78 (s, 3H). | |
| 106 | ¹H NMR (400 MHz, methanol-d4) δ 8.17 (s, 1H), 8.07 (s, 1H), | 572.1 |
| | 8.00 (s, 1H), 7.74 (dd, J = 1.2, 8.0 Hz, 1H), 7.49 (t, J = 1.6 Hz, | |
| | 1H), 7.40 (t, J = 8.0 Hz, 1H), 6.84 (d, J = 8.4 Hz, 1H), 5.09 (s, | |
| | 2H), 3.86 (s, 2H), 3.37 (s, 2H), 3.30-3.22 (m, 2H), 3.13-2.96 | |
| | (m, 2H), 2.82 (t, J = 7.2 Hz, 2H), 2.78 (s, 3H), 2.56 (s, 2H), | |
| | 1.88 (t, J = 6.8 Hz, 2H), 0.58 (d, J = 2.0 Hz, 4H). | |
| 107 | ¹H NMR (400 MHz, methanol-d4) δ 8.17 (s, 1H), 8.04 (s, 1H), | 564.1 |
| | 7.93 (s, 1H), 7.74 (dd, J = 1.2, 8.0 Hz, 1H), 7.48 (t, J = 1.6 Hz, | |
| | 1H), 7.40 (t, J = 8.0 Hz, 1H), 6.85 (d, J = 8.0 Hz, 1H), 5.09 (s, 2H), | |
| | 3.91 (s, 2H), 3.43 (s, 2H), 3.39-3.36 (m, 4H), 3.30-3.29 (m, 2H), | |
| | 3.13-2.96 (m, 2H), 2.78 (s, 3H), 1.64-1.53 (d, J = 21.6 Hz, 3H). | |
| 108 | ¹H NMR (400 MHz, DMSO-d6): δ 8.35 (s, 1H), 7.99 (s, 1H), | 562.1 |
| | 7.95-7.89 (m, 2H), 7.54 (s, 1H), 7.38 (t, J = 8.0 Hz, 1H), 6.97 | |
| | (d, J = 7.6 Hz, 1H), 6.27 (d, J = 45.6 Hz, 1H), 5.37 (d, J = 6.8 | |
| | Hz, 1H), 5.21 (d, J = 6.4 Hz, 1H), 5.16-5.03 (m, 3H), 4.83 | |
| | (dd, J = 4.0, 6.0 Hz, 1H), 3.70 (q, J = 6.4 Hz, 1H), 3.42-3.36 | |
| | (m, 1H), 3.24 (d, J = 8.0 Hz, 1H), 3.17 (s, 3H), 3.15-3.07 (m, | |
| | 2H), 1.59-1.54 (d, J = 22.4 Hz, 3H), 1.20 (d, J = 6.4 Hz, 3H). | |
| 109 | ¹H NMR (400 MHz, DMSO-d6): δ 8.35 (s, 1H), 7.99 (s, 1H), | 562.1 |
| | 7.95-7.89 (m, 2H), 7.55 (br s, 1H), 7.38 (t, J = 8.0 Hz, 1H), | |
| | 6.97 (d, J = 7.6 Hz, 1H), 6.27 (d, J = 45.6 Hz, 1H), 5.37 (d, J = | |
| | 6.4 Hz, 1H), 5.21 (d, J = 6.4 Hz, 1H), 5.16-5.05 (m, 3H), | |
| | 4.83 (dd, J = 4.0, 6.0 Hz, 1H), 3.70 (q, J = 6.4 Hz, 1H), 3.42- | |
| | 3.35 (m, 1H), 3.24 (d, J = 8.0 Hz, 1H), 3.17 (s, 3H), 3.15-3.07 | |
| | (m, 2H), 1.54 (d, J = 22.4 Hz, 3H), 1.20 (d, J = 6.4 Hz, 3H). | |
| 110 | ¹H NMR (400 MHz, methanol-d4): δ 8.28 (s, 1H), 8.07 (s, 1H), | 562.2 |
| | 7.96 (s, 1H), 7.78 (dd, J = 1.6, 8.0 Hz, 1H), 7.52 (t, J = 1.6 Hz, | |
| | 1H), 7.43 (t, J = 8.0 Hz, 1H), 6.93 (d, J = 8.0 Hz, 1H), 6.31 (d, | |
| | J = 45.2 Hz, 1H), 5.48 (d, J = 6.8 Hz, 1H), 5.32 (d, J = 6.4 Hz, | |
| | 1H), 5.22 (dd, J = 1.6, 6.4 Hz, 1H), 5.09 (br s, 2H), 5.02 (dd, | |
| | J = 4.4, 6.4 Hz, 1H), 3.71-3.69 (m, 1H), 3.46-3.37 (m, 1H), | |
| | 3.36 (d, J = 9.2 Hz, 1H), 3.28-3.15 (m, 2H), 3.12 (s, 3H), 1.58 | |
| | (d, J = 22 Hz, 3H), 1.31 (d, J = 6.4 Hz, 3H). | |
| 111 | ¹H NMR (400 MHz, methanol-d4): δ 8.28 (s, 1H), 8.08 (s, 1H), | 562.2 |
| | 7.97 (s, 1H), 7.78 (dd, J = 1.6, 8.0 Hz, 1H), 7.52 (t, J = 2.0 Hz, | |
| | 1H), 7.43 (t, J = 8.0 Hz, 1H), 6.94 (d, J = 8.0 Hz, 1H), 6.31 (d, | |
| | J = 45.2 Hz, 1H), 5.48 (d, J = 6.4 Hz, 1H), 5.32 (d, J = 6.4 Hz, | |
| | 1H), 5.22 (dd, J = 1.6, 6.4 Hz, 1H), 5.09 (br s, 2H), 5.02 (dd, | |
| | J = 4.0, 6.4 Hz, 1H), 3.84-3.72 (m, 1H), 3.58-3.47 (m, 1H), | |
| | 3.45-3.34 (m, 2H), 3.28-3.20 (m, 1H), 3.12 (s, 3H), 1.59 (d, | |
| | J = 22 Hz, 3H), 1.31 (d, J = 6.4 Hz, 3H). | |
| 116 | ¹H NMR (400 MHz, CDCl3): δ 7.91 (s, 1H), 7.63 (br s, 2H), | 529.0 |
| | 7.55 (s, 3H), 7.45-7.37 (m, 2H), 6.80 (d, J = 8.0 Hz, 1H), 6.46 | |
| | (d, J = 46 Hz, 1H), 5.36-5.27 (m, 2H), 5.26-5.19 (m, 1H), | |
| | 5.04-4.84 (m, 3H), 3.04 (s, 3H). | |
| 117 | ¹H NMR (400 MHz, CDCl3): δ 7.91 (s, 1H), 7.65-7.60 (m, | 529.0 |
| | 2H), 7.55 (s, 3H), 7.44-7.35 (m, 2H), 6.80 (d, J = 7.6 Hz, 1H), | |
| | 6.46 (d, J = 45.6 Hz, 1H), 5.34-5.20 (m, 3H), 5.02-4.82 (m, | |
| | 3H), 3.04 (d, J = 1.6 Hz, 1H). | |
| 118 | ¹H NMR (400 MHz, CDCl3): δ 8.07 (s, 1H), 7.91 (s, 2H), 7.66 | 574.2 |
| | (dd, J = 1.2, 8.0 Hz, 1H), 7.46 (br s, 1H), 7.38 (t, J = 8.0 Hz, | |
| | 1H), 6.78 (d, J = 7.2 Hz, 1H), 6.47 (d, J = 46 Hz, 1H), 5.34- | |
| | 5.27 (m, 2H), 5.24 (d, J = 6.8 Hz, 1H), 5.03-4.90 (m, 3H), | |
| | 3.94-3.76 (m, 5H), 3.04 (d, J = 1.2 Hz, 3H), 2.90-2.72 (m, | |
| | 2H), 2.71-2.62 (m, 1H), 2.51-2.36 (m, 1H), 2.00-1.81 (m, | |
| | 2H), 1.10 (d, J = 6.4 Hz, 3H). | |
| 119 | ¹H NMR (400 MHz, CDCl3): δ 8.07 (s, 1H), 7.91 (s, 2H), 7.66 | 574.2 |
| | (dd, J = 1.2, 8.0 Hz, 1H), 7.46 (br s, 1H), 7.38 (t, J = 8.0 Hz, | |
| | 1H), 6.78 (d, J = 7.6 Hz, 1H), 6.47 (d, J = 46 Hz, 1H), 5.35- | |
| | 5.27 (m, 2H), 5.26-5.22 (m, 1H), 5.03-4.86 (m, 3H), 3.96-3.76 | |
| | (m, 5H), 3.04 (d, J = 1.2 Hz, 3H), 2.86-2.61 (m, 3H), 2.43 (dd, | |
| | J = 8.8, 13.2 Hz, 1H), 2.03-1.79 (m, 2H), 1.10 (d, J = 6.4 Hz, 3H). | |
| 120 | ¹H NMR (400 MHz, CDCl3): δ 8.07 (s, 1H), 7.91 (s, 2H), 7.66 | 574.2 |
| | (dd, J = 1.6, 8.0 Hz, 1H), 7.46 (br s, 1H), 7.38 (t, J = 8.0 Hz, | |
| | 1H), 6.78 (d, J = 8.0 Hz, 1H), 6.47 (d, J = 46 Hz, 1H), 5.33- | |
| | 5.28 (m, 2H), 5.24 (d, J = 6.4 Hz, 1H), 5.02-4.86 (m, 3H), | |
| | 3.94-3.74 (m, 5H), 3.04 (d, J = 1.6 Hz, 3H), 2.89-2.60 (m, | |
| | 3H), 2.44 (dd, J = 9.2, 13.2 Hz, 1H), 2.00-1.79 (m, 2H), 1.10 | |
| | (d, J = 6.4 Hz, 3H). | |
| 125 | ¹H NMR (400 MHz, DMSO-d6) δ 8.33 (s, 1H), 8.06-7.55 (m, | 544.3 |
| | 4H), 7.32 (t, J = 7.9 Hz, 1H), 7.07 (d, J = 8.0 Hz, 1H), 5.76 (d, | |
| | J = 44.5 Hz, 1H), 5.17-4.96 (m, 2H), 4.68 (t, J = 5.5 Hz, 1H), | |
| | 3.74 (s, 2H), 3.37 (d, J = 5.4 Hz, 2H), 3.20 (s, 3H), 3.07 (d, J = | |
| | 6.8 Hz, 2H), 2.80 (d, J = 6.8 Hz, 2H), 1.23-1.00 (m, 7H). | |
| 126 | ¹H NMR (400 MHz, DMSO-d6) δ 8.33 (s, 1H), 8.06-7.55 (m, | 544.2 |
| | 4H), 7.32 (t, J = 7.9 Hz, 1H), 7.07 (d, J = 8.0 Hz, 1H), 5.76 (d, | |
| | J = 44.5 Hz, 1H), 5.17-4.96 (m, 2H), 4.68 (t, J = 5.5 Hz, 1H), | |
| | 3.74 (s, 2H), 3.37 (d, J = 5.4 Hz, 2H), 3.20 (s, 3H), 3.07 (d, J = | |
| | 6.8 Hz, 2H), 2.80 (d, J = 6.8 Hz, 2H), 1.23-1.00 (m, 7H). | |
| 127 | ¹H NMR (400 MHz, DMSO-d6) δ 8.43 (s, 1H), 8.03-7.81 (m, | 564.3 |
| | 4H), 7.43 (t, J = 8.0 Hz, 1H), 7.15 (s, 1H), 6.33 (dd, J = 42.4, | |
| | 22.7 Hz, 1H), 5.25-5.05 (m, 2H), 3.80 (s, 2H), 3.53 (s, 3H), | |
| | 2.75-2.62 (m, 2H), 2.46 (s, 2H), 1.95 (dd, J = 23.5, 1.7 Hz, | |
| | 3H), 1.77 (t, J = 6.8 Hz, 2H), 0.56-0.46 (m, 4H). | |
| 128 | ¹H NMR (400 MHz, DMSO-d6) δ 8.51 (s, 1H), 8.07-7.94 (m, | 564.3 |
| | 4H), 7.48 (t, J = 8.1 Hz, 1H), 7.27 (d, J = 7.8 Hz, 1H), 6.41 | |
| | (dd, J = 43.5, 21.6 Hz, 1H), 5.29-5.13 (m, 2H), 3.80 (s, 2H), | |
| | 3.50 (s, 3H), 2.70 (t, J = 6.8 Hz, 2H), 2.46 (s, 2H), 1.86-1.72 | |
| | (m, 5H), 0.66-0.35 (m, 4H). | |
| 129 | ¹H NMR (400 MHz, DMSO-d6) δ 8.51 (s, 1H), 7.98 (dd, J = | 564.3 |
| | 11.2, 10.0 Hz, 4H), 7.48 (t, J = 8.1 Hz, 1H), 7.27 (d, J = 8.0 | |
| | Hz, 1H), 6.41 (dd, J = 43.5, 21.6 Hz, 1H), 5.32-4.98 (m, 2H), | |
| | 3.81 (s, 2H), 3.50 (s, 3H), 2.70 (t, J = 6.8 Hz, 2H), 2.46 (s, | |
| | 2H), 1.88-1.70 (m, 5H), 0.56-0.44 (m, 4H). | |
| 130 | ¹H NMR (400 MHz, DMSO-d6) δ 8.41 (s, 1H), 8.11-7.81 (m, | 564.3 |
| | 4H), 7.41 (t, J = 8.0 Hz, 1H), 7.14 (d, J = 7.8 Hz, 1H), 6.31 | |
| | (dd, J = 42.5, 22.7 Hz, 1H), 5.11 (t, J = 11.5 Hz, 2H), 3.78 (s, | |
| | 2H), 3.51 (s, 3H), 2.75-2.56 (m, 2H), 2.38 (s, 2H), 1.93 (dd, J = | |
| | 23.5, 1.8 Hz, 3H), 1.75 (t, J = 6.8 Hz, 2H), 0.58-0.38 (m, 4H). | |
| 131 | ¹H NMR (400 MHz, DMSO-d6) δ 8.35 (s, 1H), 7.98 (s, 1H), | 560.3 |
| | 7.96-7.90 (m, 2H), 7.56 (s, 1H), 7.38 (t, J = 8.0 Hz, 1H), 6.97 | |
| | (d, J = 7.6 Hz, 1H), 6.28 (d, J = 45.8 Hz, 1H), 5.37 (d, J = 6.7 | |
| | Hz, 1H), 5.21 (d, J = 5.9 Hz, 1H), 5.19-4.99 (m, 3H), 4.88- | |
| | 4.77 (m, 1H), 3.96-3.85 (m, 1H), 3.84-3.71 (m, 2H), 3.18 | |
| | (s, 3H), 3.16 (s, 3H), 2.69 (dd, J = 10.0, 6.3 Hz, 1H), 2.61 (dd, | |
| | J = 15.0, 7.7 Hz, 1H), 2.47-2.37 (m, 1H), 2.10-1.95 (m, | |
| | 1H), 1.76-1.62 (m, 1H). | |
| 132 | ¹H NMR (400 MHz, DMSO-d6) δ 8.35 (s, 1H), 7.98 (s, 1H), | 560.3 |
| | 7.96-7.89 (m, 2H), 7.56 (s, 1H), 7.38 (t, J = 8.0 Hz, 1H), 6.97 | |
| | (d, J = 7.7 Hz, 1H), 6.28 (d, J = 45.8 Hz, 1H), 5.37 (d, J = 6.7 | |
| | Hz, 1H), 5.22 (d, J = 6.1 Hz, 1H), 5.17-5.00 (m, 3H), 4.83 | |
| | (dd, J = 6.0, 4.1 Hz, 1H), 3.96-3.84 (m, 1H), 3.84-3.71 (m, | |
| | 2H), 3.18 (s, 3H), 3.16 (s, 3H), 2.69 (dd, J = 10.0, 6.2 Hz, 1H), | |
| | 2.61 (dd, J = 14.9, 7.8 Hz, 1H), 2.48-2.37 (m, 1H), 2.01 (td, | |
| | J = 13.8, 7.5 Hz, 1H), 1.74-1.61 (m, 1H). | |
| 133 | ¹H NMR (400 MHz, DMSO-d6) δ 8.33 (s, 1H), 7.98 (s, 1H), | 592.3 |
| | 7.94-7.87 (m, 2H), 7.53 (s, 1H), 7.36 (t, J = 8.0 Hz, 1H), 6.95 | |
| | (d, J = 7.7 Hz, 1H), 6.26 (d, J = 45.8 Hz, 1H), 5.35 (d, J = 6.6 | |
| | Hz, 1H), 5.20 (d, J = 6.1 Hz, 1H), 5.16-5.02 (m, 3H), 4.97- | |
| | 4.88 (m, 1H), 4.81 (dd, J = 6.0, 4.1 Hz, 1H), 3.70 (s, 2H), 3.41 | |
| | (d, J = 4.4 Hz, 1H), 3.36 (d, J = 4.7 Hz, 1H), 3.16 (s, 3H), 2.70- | |
| | 2.55 (m, 2H), 2.27 (dd, J = 22.0, 11.1 Hz, 2H), 1.77-1.65 | |
| | (m, 3H), 1.65-1.52 (m, 1H). | |
| 134 | ¹H NMR (400 MHz, DMSO-d6) δ 8.35 (s, 1H), 8.00 (s, 1H), | 592.3 |
| | 7.97-7.88 (m, 2H), 7.55 (s, 1H), 7.38 (t, J = 8.0 Hz, 1H), 6.97 | |
| | (d, J = 7.7 Hz, 1H), 6.28 (d, J = 45.8 Hz, 1H), 5.37 (d, J = 6.7 | |
| | Hz, 1H), 5.21 (d, J = 6.1 Hz, 1H), 5.17-5.05 (m, 3H), 4.93 (t, | |
| | J = 5.6 Hz, 1H), 4.86-4.78 (m, 1H), 3.72 (s, 2H), 3.43 (d, J = | |
| | 4.9 Hz, 1H), 3.40-3.35 (m, 1H), 3.18 (s, 3H), 2.69-2.56 | |
| | (m, 2H), 2.29 (dd, J = 21.5, 10.5 Hz, 2H), 1.79-1.66 (m, 3H), | |
| | 1.66-1.53 (m, 1H). | |
| 135 | ¹H NMR (400 MHz, DMSO-d6) δ 8.35 (s, 1H), 7.98-7.93 (m, | 578.2 |
| | 2H), 7.91 (s, 1H), 7.56 (t, J = 1.9 Hz, 1H), 7.38 (t, J = 8.0 Hz, | |
| | 1H), 6.97 (d, J = 8.3 Hz, 1H), 6.28 (d, J = 45.9 Hz, 1H), 5.37 | |
| | (d, J = 6.5 Hz, 1H), 5.21 (d, J = 6.2 Hz, 1H), 5.18-5.04 (m, | |
| | 3H), 4.83 (dd, J = 6.2, 4.0 Hz, 1H), 3.88 (s, 2H), 3.69 (s, 1H), | |
| | 3.63 (s, 1H), 3.49-3.38 (m, 2H), 3.33 (s, 3H), 3.27-3.21 (m, | |
| | 1H), 3.21-3.15 (m, 4H). | |
| 136 | ¹H NMR (400 MHz, DMSO-d6) δ 8.35 (s, 1H), 7.98-7.92 (m, | 578.1 |
| | 2H), 7.91 (s, 1H), 7.56 (t, J = 1.8 Hz, 1H), 7.38 (t, J = 8.0 Hz, | |
| | 1H), 6.97 (d, J = 8.3 Hz, 1H), 6.28 (d, J = 45.9 Hz, 1H), 5.37 | |
| | (d, J = 6.6 Hz, 1H), 5.22 (d, J = 6.3 Hz, 1H), 5.19-5.02 (m, | |
| | 3H), 4.83 (dd, J = 6.2, 4.0 Hz, 1H), 3.88 (s, 2H), 3.69 (s, 1H), | |
| | 3.63 (s, 1H), 3.43 (dd, J = 13.5, 9.4 Hz, 2H), 3.33 (s, 3H), 3.27- | |
| | 3.22 (m, 1H), 3.22-3.14 (m, 4H). | |
| 137 | ¹H NMR (400 MHz, DMSO-d6) δ 8.33 (s, 1H), 7.95-7.86 (m, | 566.2 |
| | 3H), 7.54 (t, J = 1.8 Hz, 1H), 7.36 (t, J = 8.0 Hz, 1H), 6.95 (d, | |
| | J = 7.8 Hz, 1H), 6.42-6.07 (m, 2H), 5.35 (d, J = 6.7 Hz, 1H), | |
| | 5.20 (d, J = 6.2 Hz, 1H), 5.15-5.01 (m, 3H), 4.81 (dd, J = 6.2, | |
| | 4.0 Hz, 1H), 3.77 (s, 2H), 3.36-3.32 (m, 3H), 3.18-3.10 (m, 5H). | |
| 138 | ¹H NMR (400 MHz, DMSO-d6) δ 8.33 (s, 1H), 7.96-7.85 (m, | 566.2 |
| | 3H), 7.54 (s, 1H), 7.36 (t, J = 8.0 Hz, 1H), 6.95 (d, J = 7.9 Hz, | |
| | 1H), 6.43-6.05 (m, 2H), 5.35 (d, J = 6.6 Hz, 1H), 5.20 (d, J = | |
| | 6.3 Hz, 1H), 5.14-5.01 (m, 3H), 4.81 (dd, J = 6.2, 3.9 Hz, | |
| | 1H), 3.77 (s, 2H), 3.36-3.32 (m, 3H), 3.18-3.08 (m, 5H). | |
| 139 | ¹H NMR (400 MHz, DMSO-d6) δ 8.35 (s, 1H), 7.94-7.91 (m, | 558.2 |
| | 2H), 7.88 (s, 1H), 7.56 (t, J = 1.7 Hz, 1H), 7.38 (t, J = 8.0 Hz, | |
| | 1H), 6.97 (d, J = 7.9 Hz, 1H), 6.28 (d, J = 45.8 Hz, 1H), 5.37 | |
| | (d, J = 6.8 Hz, 1H), 5.21 (d, J = 5.9 Hz, 1H), 5.16-5.04 (m, | |
| | 3H), 4.83 (dd, J = 6.2, 4.0 Hz, 1H), 4.37 (t, J = 7.5 Hz, 2H), | |
| | 3.75 (s, 2H), 3.54-3.51 (m, 2H), 3.18 (s, 3H), 3.17-3.14 (m, | |
| | 2H), 2.78 (t, J = 7.5 Hz, 2H). | |
| 140 | ¹H NMR (400 MHz, DMSO-d6) δ 8.35 (s, 1H), 7.94-7.91 (m, | 558.2 |
| | 2H), 7.88 (s, 1H), 7.56 (t, J = 1.7 Hz, 1H), 7.38 (t, J = 8.0 Hz, | |
| | 1H), 6.97 (d, J = 7.9 Hz, 1H), 6.28 (d, J = 45.8 Hz, 1H), 5.37 | |
| | (d, J = 6.8 Hz, 1H), 5.21 (d, J = 5.9 Hz, 1H), 5.16-5.04 (m, | |
| | 3H), 4.83 (dd, J = 6.2, 4.0 Hz, 1H), 4.37 (t, J = 7.5 Hz, 2H), | |
| | 3.75 (s, 2H), 3.54-3.51 (m, 2H), 3.18 (s, 3H), 3.17-3.14 (m, | |
| | 2H), 2.78 (t, J = 7.5 Hz, 2H). | |
| 141 | ¹H NMR (400 MHz, DMSO-d6) δ 8.33 (s, 1H), 7.99-7.88 (m, | 574.3 |
| | 3H), 7.53 (s, 1H), 7.36 (t, J = 8.0 Hz, 1H), 6.95 (d, J = 7.7 Hz, | |
| | 1H), 6.26 (d, J = 45.8 Hz, 1H), 5.35 (d, J = 6.9 Hz, 1H), 5.20 | |
| | (d, J = 6.1 Hz, 1H), 5.15-5.01 (m, 3H), 4.81 (dd, J = 6.1, 4.0 | |
| | Hz, 1H), 4.08 (s, 1H), 3.66 (s, 2H), 3.16 (s, 3H), 2.44-2.34 | |
| | (m, 4H), 1.52-1.40 (m, 4H), 1.09 (s, 3H). | |
| 142 | ¹H NMR (400 MHz, DMSO-d6) δ 8.33 (s, 1H), 8.00-7.87 (m, | 574.3 |
| | 3H), 7.53 (s, 1H), 7.36 (t, J = 8.0 Hz, 1H), 6.95 (d, J = 7.7 Hz, | |
| | 1H), 6.26 (d, J = 45.7 Hz, 1H), 5.35 (d, J = 6.8 Hz, 1H), 5.20 | |
| | (d, J = 6.1 Hz, 1H), 5.16-5.01 (m, 3H), 4.81 (dd, J = 6.3, 4.2 | |
| | Hz, 1H), 4.08 (s, 1H), 3.66 (s, 2H), 3.16 (s, 3H), 2.44-2.34 | |
| | (m, 4H), 1.54-1.41 (m, 4H), 1.09 (s, 3H). | |
| 143 | ¹H NMR (400 MHz, DMSO-d6) δ 8.35 (s, 1H), 7.97 (s, 1H), | 574.3 |
| | 7.96-7.89 (m, 2H), 7.56 (t, J = 1.8 Hz, 1H), 7.38 (t, J = 8.0 | |
| | Hz, 1H), 6.97 (d, J = 7.9 Hz, 1H), 6.28 (d, J = 45.8 Hz, 1H), | |
| | 5.37 (d, J = 6.5 Hz, 1H), 5.21 (d, J = 5.5 Hz, 1H), 5.19-5.02 | |
| | (m, 3H), 4.83 (dd, J = 6.2, 4.0 Hz, 1H), 3.66 (q, J = 14.0 Hz, | |
| | 2H), 3.20-3.15 (m, 4H), 2.84 (d, J = 9.6 Hz, 1H), 2.79-2.65 (m, | |
| | 2H), 1.98 (t, J = 10.2 Hz, 1H), 1.71 (t, J = 9.9 Hz, 1H), 1.61 (d, | |
| | J = 9.0 Hz, 3H), 1.56-1.41 (m, 1H), 0.90 (d, J = 9.8 Hz, 1H). | |
| 144 | ¹H NMR (400 MHz, DMSO-d6) δ 8.35 (s, 1H), 7.97 (s, 1H), | 574.2 |
| | 7.96-7.90 (m, 2H), 7.56 (s, 1H), 7.38 (t, J = 8.0 Hz, 1H), 6.97 | |
| | (d, J = 7.8 Hz, 1H), 6.28 (d, J = 45.8 Hz, 1H), 5.37 (d, J = 6.6 | |
| | Hz, 1H), 5.21 (d, J = 6.0 Hz, 1H), 5.19-5.02 (m, 3H), 4.83 | |
| | (dd, J = 6.0, 4.0 Hz, 1H), 4.39 (t, J = 4.8 Hz, 1H), 3.66 (q, J = | |
| | 13.8 Hz, 2H), 3.30-3.24 (m, 1H), 3.23-3.09 (m, 4H), 2.84 | |
| | (d, J = 10.0 Hz, 1H), 2.73 (d, J = 10.9 Hz, 1H), 1.98 (t, J = | |
| | 10.2 Hz, 1H), 1.71 (t, J = 10.2 Hz, 1H), 1.62 (d, J = 9.3 Hz, | |
| | 3H), 1.55-1.40 (m, 1H), 0.99-0.81 (m, 1H). | |
| 145 | ¹H NMR (400 MHz, DMSO-d6) δ 8.35 (s, 1H), 7.97 (s, 1H), | 588.3 |
| | 7.95-7.87 (m, 2H), 7.55 (t, J = 1.8 Hz, 1H), 7.38 (t, J = 8.0 | |
| | Hz, 1H), 6.97 (d, J = 7.9 Hz, 1H), 6.28 (d, J = 45.9 Hz, 1H), | |
| | 5.37 (d, J = 6.6 Hz, 1H), 5.22 (d, J = 6.4 Hz, 1H), 5.18-5.00 (m, | |
| | 3H), 4.83 (dd, J = 6.2, 4.0 Hz, 1H), 3.66 (s, 2H), 3.22 (s, 3H), | |
| | 3.20-3.13 (m, 5H), 2.84-2.75 (m, 2H), 1.98 (t, J = 10.7 Hz, 2H), | |
| | 1.62 (d, J = 11.6 Hz, 2H), 1.59-1.46 (m, 1H), 1.26-1.13 (m, 2H). | |
| 146 | ¹H NMR (400 MHz, DMSO-d6) δ 8.35 (s, 1H), 7.97 (s, 1H), | 588.3 |
| | 7.96-7.88 (m, 2H), 7.55 (s, 1H), 7.38 (t, J = 8.0 Hz, 1H), 6.97 | |
| | (d, J = 7.8 Hz, 1H), 6.28 (d, J = 45.8 Hz, 1H), 5.37 (d, J = 6.7 | |
| | Hz, 1H), 5.21 (d, J = 6.1 Hz, 1H), 5.16-5.03 (m, 3H), 4.83 | |
| | (dd, J = 6.0, 4.0 Hz, 1H), 3.66 (s, 2H), 3.22 (s, 3H), 3.20-3.15 | |
| | (m, 5H), 2.80 (d, J = 11.2 Hz, 2H), 1.98 (t, J = 10.7 Hz, 2H), | |
| | 1.62 (d, J = 11.9 Hz, 2H), 1.58-1.43 (m, 1H), 1.33-1.09 (m, 2H). | |
| 147 | ¹H NMR (400 MHz, DMSO-d6) δ 8.35 (s, 1H), 8.01 (s, 1H), | 560.1 |
| | 7.94 (d, J = 11.7 Hz, 2H), 7.54 (s, 1H), 7.38 (t, J = 8.0 Hz, | |
| | 1H), 6.97 (d, J = 7.7 Hz, 1H), 6.27 (d, J = 45.8 Hz, 1H), 5.37 | |
| | (d, J = 6.7 Hz, 1H), 5.21 (d, J = 6.1 Hz, 1H), 5.18-4.99 (m, | |
| | 3H), 4.83 (dd, J = 5.9, 4.1 Hz, 1H), 4.55 (bs, 1H), 4.25 (d, J = | |
| | 13.8 Hz, 1H), 3.59 (d, J = 13.9 Hz, 1H), 3.46 (dd, J = 10.7, 4.9 | |
| | Hz, 1H), 3.18 (s, 3H), 2.82-2.59 (m, 3H), 2.20 (q, J = 8.5 Hz, | |
| | 1H), 1.84 (dd, J = 17.3, 6.6 Hz, 1H), 1.73-1.50 (m, 3H). | |
| | Contains trace amounts of diethylamine. | |
| 148 | ¹H NMR (400 MHz, DMSO-d6) δ 8.35 (s, 1H), 8.01 (s, 1H), | 560.2 |
| | 7.94 (d, J = 11.4 Hz, 2H), 7.54 (s, 1H), 7.38 (t, J = 8.0 Hz, | |
| | 1H), 6.97 (d, J = 7.7 Hz, 1H), 6.28 (d, J = 45.8 Hz, 1H), 5.37 | |
| | (d, J = 6.7 Hz, 1H), 5.21 (d, J = 6.1 Hz, 1H), 5.18-5.00 (m, | |
| | 3H), 4.83 (dd, J = 5.9, 4.1 Hz, 1H), 4.54 (s, 1H), 4.25 (d, J = | |
| | 13.8 Hz, 1H), 3.59 (d, J = 13.9 Hz, 1H), 3.54-3.41 (m, 1H), 3.18 | |
| | (s, 3H), 2.77 (d, J = 2.9 Hz, 1H), 2.70-2.58 (m, 1H), 2.20 (q, J = | |
| | 8.6 Hz, 1H), 1.84 (dd, J = 17.3, 6.5 Hz, 1H), 1.74-1.50 (m, 3H). | |
| 149 | ¹H NMR (400 MHz, DMSO-d6) δ 8.35 (s, 1H), 8.01 (s, 1H), | 560.2 |
| | 7.94 (d, J = 11.4 Hz, 2H), 7.54 (s, 1H), 7.38 (t, J = 8.0 Hz, | |
| | 1H), 6.97 (d, J = 7.8 Hz, 1H), 6.28 (d, J = 45.8 Hz, 1H), 5.37 | |
| | (d, J = 6.7 Hz, 1H), 5.21 (d, J = 6.0 Hz, 1H), 5.19-5.02 (m, | |
| | 3H), 4.83 (dd, J = 5.9, 4.1 Hz, 1H), 4.54 (s, 1H), 4.25 (d, J = | |
| | 13.9 Hz, 1H), 3.59 (d, J = 13.9 Hz, 1H), 3.52-3.41 (m, 1H), | |
| | 3.18 (s, 3H), 2.82-2.72 (m, 1H), 2.68-2.58 (m, 1H), 2.20 | |
| | (dd, J = 16.4, 8.6 Hz, 1H), 1.84 (dd, J = 17.4, 6.6 Hz, 1H), | |
| | 1.61 (ddd, J = 18.1, 12.9, 7.0 Hz, 3H). | |
| 150 | ¹H NMR (400 MHz, DMSO-d6) δ 8.35 (s, 1H), 8.01 (s, 1H), | 560.2 |
| | 7.94 (d, J = 11.5 Hz, 2H), 7.54 (s, 1H), 7.38 (t, J = 8.0 Hz, | |
| | 1H), 6.97 (d, J = 7.7 Hz, 1H), 6.27 (d, J = 45.8 Hz, 1H), 5.37 | |
| | (d, J = 6.7 Hz, 1H), 5.21 (d, J = 6.0 Hz, 1H), 5.17-5.01 (m, | |
| | 3H), 4.83 (dd, J = 6.0, 4.0 Hz, 1H), 4.54 (s, 1H), 4.25 (d, J = | |
| | 13.9 Hz, 1H), 3.59 (d, J = 13.9 Hz, 1H), 3.50-3.41 (m, 1H), | |
| | 3.18 (s, 3H), 2.81-2.71 (m, 1H), 2.68-2.58 (m, 1H), 2.20 | |
| | (q, J = 8.7 Hz, 1H), 1.84 (dd, J = 17.2, 6.6 Hz, 1H), 1.61 (ddd, | |
| | J = 17.8, 12.9, 7.0 Hz, 3H). | |
| 151 | ¹H NMR (400 MHz, DMSO-d6) δ 8.35 (s, 1H), 7.98 (s, 1H), | 574.2 |
| | 7.94-7.87 (m, 2H), 7.56 (s, 1H), 7.38 (t, J = 8.0 Hz, 1H), 6.97 | |
| | (d, J = 7.7 Hz, 1H), 6.28 (d, J = 45.8 Hz, 1H), 5.37 (d, J = 6.7 | |
| | Hz, 1H), 5.21 (d, J = 6.0 Hz, 1H), 5.17-5.03 (m, 3H), 4.83 | |
| | (dd, J = 6.0, 4.0 Hz, 1H), 4.40 (t, J = 4.9 Hz, 1H), 3.66 (q, J = | |
| | 14.0 Hz, 2H), 3.25-3.10 (m, 4H), 2.84 (d, J = 9.3 Hz, 1H), | |
| | 2.78-2.68 (m, 1H), 1.98 (t, J = 11.0 Hz, 1H), 1.78-1.55 (m, | |
| | 4H), 1.53-1.36 (m, 1H), 1.03-0.78 (m, 1H). | |
| 152 | ¹H NMR (400 MHz, DMSO-d6) δ 8.35 (s, 1H), 7.98 (s, 1H), | 574.2 |
| | 7.95-7.88 (m, 2H), 7.56 (s, 1H), 7.38 (t, J = 8.0 Hz, 1H), 6.97 | |
| | (d, J = 7.7 Hz, 1H), 6.28 (d, J = 45.8 Hz, 1H), 5.37 (d, J = 6.7 | |
| | Hz, 1H), 5.21 (d, J = 6.1 Hz, 1H), 5.18-4.97 (m, 3H), 4.83 | |
| | (dd, J = 6.0, 4.0 Hz, 1H), 4.40 (t, J = 4.4 Hz, 1H), 3.66 (q, J = | |
| | 13.6 Hz, 2H), 3.24-3.03 (m, 4H), 2.84 (d, J = 9.2 Hz, 1H), | |
| | 2.73 (d, J = 10.0 Hz, 1H), 1.98 (t, J = 10.0 Hz, 1H), 1.80- | |
| | 1.55 (m, 4H), 1.54-1.37 (m, 1H), 1.01-0.81 (m, 1H). | |
| 153 | ¹H NMR (400 MHz, DMSO-d6) δ 8.35 (s, 1H), 7.98 (s, 1H), | 578.3 |
| | 7.95-7.89 (m, 2H), 7.56 (s, 1H), 7.38 (t, J = 8.0 Hz, 1H), 6.97 | |
| | (d, J = 7.8 Hz, 1H), 6.28 (d, J = 45.8 Hz, 1H), 5.37 (d, J = 6.7 | |
| | Hz, 1H), 5.21 (d, J = 6.0 Hz, 1H), 5.19-5.03 (m, 3H), 4.83 | |
| | (dd, J = 6.0, 4.0 Hz, 1H), 3.90 (s, 2H), 3.41 (d, J = 7.5 Hz, 2H), | |
| | 3.36 (d, J = 7.7 Hz, 2H), 3.18 (s, 3H), 1.59 (t, J = 8.7 Hz, 2H). | |
| 154 | ¹H NMR (400 MHz, DMSO-d6) δ 8.35 (s, 1H), 7.98 (s, 1H), | 578.3 |
| | 7.95-7.87 (m, 2H), 7.56 (s, 1H), 7.38 (t, J = 8.0 Hz, 1H), 6.97 | |
| | (d, J = 7.7 Hz, 1H), 6.28 (d, J = 45.8 Hz, 1H), 5.37 (d, J = 6.7 | |
| | Hz, 1H), 5.21 (d, J = 6.1 Hz, 1H), 5.18-5.03 (m, 3H), 4.83 | |
| | (dd, J = 6.0, 4.1 Hz, 1H), 3.90 (s, 2H), 3.41 (d, J = 7.6 Hz, | |
| | 2H), 3.38-3.34 (m, 2H), 3.18 (s, 3H), 1.59 (t, J = 8.7 Hz, 2H). | |
| 155 | ¹H NMR (400 MHz, DMSO-d6) δ 8.35 (s, 1H), 7.98 (s, 1H), | 574.3 |
| | 7.96-7.90 (m, 2H), 7.55 (s, 1H), 7.38 (t, J = 8.0 Hz, 1H), 6.97 | |
| | (d, J = 7.7 Hz, 1H), 6.28 (d, J = 45.8 Hz, 1H), 5.37 (d, J = 6.5 | |
| | Hz, 1H), 5.21 (d, J = 5.3 Hz, 1H), 5.19-5.02 (m, 3H), 4.83 | |
| | (dd, J = 6.2, 4.0 Hz, 1H), 4.23 (d, J = 14.1 Hz, 1H), 3.62 (d, | |
| | J = 14.0 Hz, 1H), 3.42 (dd, J = 9.5, 5.5 Hz, 1H), 3.30-3.25 | |
| | (m, 2H), 3.25 (s, 3H), 2.84-2.70 (m, 2H), 2.22 (q, J = 8.7 Hz, | |
| | 1H), 1.95-1.82 (m, 1H), 1.72-1.59 (m, 2H), 1.58-1.45 (m, | |
| | 1H). 2 aliphatic protons missing. | |
| 156 | ¹H NMR (400 MHz, DMSO-d6) 8.35 (s, 1H), 7.98 (s, 1H), | 574.3 |
| | 7.97-7.90 (m, 2H), 7.55 (s, 1H), 7.38 (t, J = 8.0 Hz, 1H), 6.97 | |
| | (d, J = 7.8 Hz, 1H), 6.28 (d, J = 45.8 Hz, 1H), 5.37 (d, J = 6.7 | |
| | Hz, 1H), 5.21 (d, J = 6.0 Hz, 1H), 5.17-5.02 (m, 3H), 4.83 | |
| | (dd, J = 6.0, 4.1 Hz, 1H), 4.23 (d, J = 14.0 Hz, 1H), 3.63 | |
| | (d, J = 14.0 Hz, 1H), 3.48-3.37 (m, 1H), 3.29-3.26 (m, 1H), | |
| | 3.25 (s, 2H), 3.18 (s, 3H), 2.85-2.70 (m, 2H), 2.22 (q, J = | |
| | 8.6 Hz, 1H), 1.94-1.82 (m, 1H), 1.72-1.59 (m, 2H), 1.58-1.44 | |
| | (m, 1H). 1 aliphatic proton missing. | |
| 157 | ¹H NMR (400 MHz, DMSO-d6) δ 8.35 (s, 1H), 7.97 (s, 1H), | 576.4 |
| | 7.96-7.89 (m, 2H), 7.55 (s, 1H), 7.38 (t, J = 8.0 Hz, 1H), 6.97 | |
| | (d, J = 7.7 Hz, 1H), 6.27 (d, J = 45.8 Hz, 1H), 5.37 (d, J = 6.7 | |
| | Hz, 1H), 5.21 (d, J = 6.0 Hz, 1H), 5.18-5.04 (m, 3H), 4.83 | |
| | (dd, J = 6.0, 4.1 Hz, 1H), 4.44 (bs, 1H), 3.68 (s, 2H), 3.18 (s, | |
| | 3H), 2.15 (s, 3H), 1.65-1.54 (m, 2H), 1.07 (s, 6H). 2 | |
| | aliphatics protons missing (probably under DMSO-D6 peak). | |
| 158 | ¹H NMR (400 MHz, DMSO-d6) δ 8.35 (s, 1H), 7.96 (d, J = 9.0 | 576.4 |
| | Hz, 1H), 7.95-7.89 (m, 2H), 7.55 (s, 1H), 7.38 (t, J = 8.0 Hz, | |
| | 1H), 6.97 (d, J = 7.7 Hz, 1H), 6.28 (d, J = 45.8 Hz, 1H), 5.37 | |
| | (d, J = 6.6 Hz, 1H), 5.21 (d, J = 6.0 Hz, 1H), 5.18-5.01 (m, | |
| | 3H), 4.83 (dd, J = 6.0, 4.0 Hz, 1H), 4.44 (bs, 1H), 3.68 (s, 2H), | |
| | 3.18 (s, 3H), 2.15 (s, 3H), 1.65-1.53 (m, 2H), 1.07 (s, 6H). 2 | |
| | aliphatic protons missing. | |
| 159 | ¹H NMR (400 MHz, DMSO-d6) δ 8.35 (s, 1H), 8.32 (s, 1H), | 574.3 |
| | 7.98 (s, 1H), 7.96-7.89 (m, 2H), 7.55 (s, 1H), 7.38 (t, J = 8.0 | |
| | Hz, 1H), 6.97 (d, J = 7.6 Hz, 1H), 6.28 (d, J = 45.9 Hz, 1H), | |
| | 5.37 (d, J = 6.7 Hz, 1H), 5.21 (d, J = 6.0 Hz, 1H), 5.18-5.02 | |
| | (m, 3H), 4.87-4.79 (m, 1H), 4.23 (d, J = 13.9 Hz, 1H), 3.63 | |
| | (d, J = 14.0 Hz, 1H), 3.28 (dd, J = 9.6, 3.7 Hz, 1H), 3.25 (s, | |
| | 3H), 3.18 (s, 3H), 2.83-2.70 (m, 2H), 2.22 (dd, J = 16.8, 8.5 | |
| | Hz, 1H), 1.94-1.82 (m, 1H), 1.66 (dd, J = 14.4, 7.8 Hz, 2H), | |
| | 1.58-1.43 (m, 1H). Many protons in DMSO-D6 peak. | |
| 160 | ¹H NMR (400 MHz, DMSO-d6) δ 8.35 (s, 1H), 7.97 (d, J = | 574.3 |
| | 12.2 Hz, 1H), 7.96-7.91 (m, 2H), 7.55 (s, 1H), 7.38 (t, J = | |
| | 8.0 Hz, 1H), 6.97 (d, J = 7.8 Hz, 1H), 6.28 (d, J = 45.8 Hz, | |
| | 1H), 5.37 (d, J = 6.7 Hz, 1H), 5.21 (d, J = 6.1 Hz, 1H), 5.18- | |
| | 5.01 (m, 3H), 4.83 (dd, J = 6.0, 4.1 Hz, 1H), 4.23 (d, J = 14.0 | |
| | Hz, 1H), 3.62 (d, J = 14.0 Hz, 1H), 3.42 (dd, J = 9.5, 5.5 Hz, | |
| | 1H), 3.32-3.26 (m, 1H), 3.25 (s, 3H), 3.18 (s, 3H), 2.83-2.70 | |
| | (m, 2H), 2.22 (q, J = 8.6 Hz, 1H), 1.96-1.80 (m, 1H), 1.72-1.60 | |
| | (m, 2H), 1.59-1.44 (m, 1H). Many peaks in DMSO-D6 peaks | |
| 161 | ¹H NMR (400 MHz, DMSO-d6) δ 8.35 (s, 1H), 8.24 (s, 1H), | 546.3 |
| | 7.98-7.87 (m, 3H), 7.57 (s, 1H), 7.38 (t, J = 8.0 Hz, 1H), 6.97 | |
| | (d, J = 7.7 Hz, 1H), 6.28 (d, J = 45.8 Hz, 1H), 5.37 (d, J = 6.6 | |
| | Hz, 1H), 5.21 (d, J = 6.1 Hz, 1H), 5.14-5.01 (m, 3H), 4.88- | |
| | 4.77 (m, 1H), 3.79 (s, 2H), 3.20 (d, J = 7.4 Hz, 2H), 3.18 (s, | |
| | 3H), 2.96 (d, J = 7.1 Hz, 2H), 1.38 (s, 3H). | |
| 162 | ¹H NMR (400 MHz, DMSO-d6) δ 8.33 (s, 1H), 8.22 (s, 1H), | 546.2 |
| | 7.95-7.83 (m, 3H), 7.55 (s, 1H), 7.36 (t, J = 8.0 Hz, 1H), 6.95 | |
| | (d, J = 7.8 Hz, 1H), 6.26 (d, J = 45.8 Hz, 1H), 5.35 (d, J = 6.7 | |
| | Hz, 1H), 5.20 (d, J = 6.0 Hz, 1H), 5.15-4.98 (m, 3H), 4.81 | |
| | (dd, J = 6.0, 4.1 Hz, 1H), 3.77 (s, 2H), 3.19 (d, J = 7.5 Hz, | |
| | 2H), 3.16 (s, 3H), 2.94 (d, J = 7.2 Hz, 3H), 1.36 (s, 3H). | |
| 163 | ¹H NMR (400 MHz, DMSO-d6) δ 8.35 (s, 1H), 7.96 (s, 1H), | 594.2 |
| | 7.93 (d, J = 5.7 Hz, 2H), 7.56 (s, 1H), 7.38 (t, J = 8.0 Hz, 1H), | |
| | 6.97 (d, J = 7.6 Hz, 1H), 6.28 (d, J = 45.8 Hz, 1H), 5.37 (d, J = | |
| | 6.7 Hz, 1H), 5.21 (d, J = 6.2 Hz, 1H), 5.18-5.01 (m, 3H), | |
| | 4.91-4.78 (m, 1H), 4.28-4.12 (m, 1H), 3.84 (s, 2H), 3.66- | |
| | 3.52 (m, 2H), 3.52-3.39 (m, 2H), 3.18 (s, 3H), 2.97 (s, 3H). | |
| | Contains 5.5% of diethylamine | |
| 164 | ¹H NMR (400 MHz, DMSO-d6) δ 8.35 (s, 1H), 7.97 (s, 1H), | 594.3 |
| | 7.93 (d, J = 5.9 Hz, 2H), 7.56 (s, 1H), 7.38 (t, J = 8.0 Hz, | |
| | 1H), 6.97 (d, J = 7.7 Hz, 1H), 6.28 (d, J = 45.8 Hz, 1H), 5.37 | |
| | (d, J = 6.7 Hz, 1H), 5.21 (d, J = 6.2 Hz, 1H), 5.19-5.02 (m, | |
| | 3H), 4.89-4.79 (m, 1H), 4.30-4.14 (m, 1H), 3.84 (s, 2H), | |
| | 3.66-3.53 (m, 2H), 3.53-3.40 (m, 2H), 3.18 (s, 3H), 2.97 | |
| | (s, J = 11.9 Hz, 3H). Contains 7.5% of diethylamine | |
| 165 | ¹H NMR (400 MHz, DMSO-d6) δ 8.35 (s, 1H), 7.92 (d, J = | 556.2 |
| | 7.5 Hz, 2H), 7.87 (s, 1H), 7.56 (s, 1H), 7.38 (t, J = 7.9 Hz, | |
| | 1H), 6.97 (d, J = 8.1 Hz, 1H), 6.28 (d, J = 45.9 Hz, 1H), 5.37 | |
| | (d, J = 6.4 Hz, 1H), 5.21 (d, J = 6.3 Hz, 1H), 5.17-5.02 (m, | |
| | 3H), 4.87-4.78 (m, 1H), 3.70 (s, 2H), 3.18 (s, 3H), 3.15 (s, | |
| | 4H), 2.06 (t, J = 7.6 Hz, 4H), 1.81-1.71 (m, 2H). | |
| 166 | ¹H NMR (400 MHz, DMSO-d6) δ 8.35 (s, 1H), 7.92 (d, J = 5.7 | 556.3 |
| | Hz, 2H), 7.87 (s, 1H), 7.56 (s, 1H), 7.38 (t, J = 8.0 Hz, 1H), | |
| | 6.97 (d, J = 7.8 Hz, 1H), 6.27 (d, J = 45.8 Hz, 1H), 5.37 (d, J = | |
| | 6.7 Hz, 1H), 5.21 (d, J = 5.9 Hz, 1H), 5.19-5.02 (m, 3H), | |
| | 4.89-4.78 (m, 1H), 3.70 (s, 2H), 3.18 (s, 3H), 3.15 (s, 4H), | |
| | 2.06 (t, J = 7.6 Hz, 4H), 1.81-1.68 (m, 2H). | |
| 167 | ¹H NMR (400 MHz, DMSO-d6) δ 8.35 (s, 1H), 8.32 (s, 1H), | 560.4 |
| | 7.97-7.86 (m, 3H), 7.57 (s, 1H), 7.38 (t, J = 8.0 Hz, 1H), 6.97 | |
| | (d, J = 8.0 Hz, 1H), 6.27 (d, J = 45.8 Hz, 1H), 5.37 (d, J = 6.5 | |
| | Hz, 1H), 5.21 (d, J = 5.6 Hz, 1H), 5.17-5.04 (m, 3H), 4.83 | |
| | (dd, J = 6.1, 4.0 Hz, 1H), 3.80 (s, 2H), 3.24 (d, J = 7.7 Hz, | |
| | 2H), 3.18 (s, 3H), 2.92 (d, J = 7.6 Hz, 2H), 1.66 (q, J = 7.3 Hz, | |
| | 2H), 0.88 (t, J = 7.4 Hz, 3H). | |
| 168 | ¹H NMR (400 MHz, DMSO-d6) δ 8.35 (s, 1H), 8.31 (s, 1H), | 560.5 |
| | 7.95-7.87 (m, 3H), 7.57 (s, 1H), 7.38 (t, J = 8.0 Hz, 1H), 6.97 | |
| | (d, J = 7.7 Hz, 1H), 6.28 (d, fJ = 45.8 Hz, 1H), 5.37 (d, J = 6.6 | |
| | Hz, 1H), 5.21 (d, J = 6.0 Hz, 1H), 5.17-5.03 (m, 3H), 4.83 | |
| | (dd, J = 6.2, 4.1 Hz, 1H), 3.80 (s, 3H), 3.24 (d, J = 7.7 Hz, | |
| | 2H), 3.18 (s, 3H), 2.92 (d, J = 7.7 Hz, 2H), 1.66 (q, J = 7.3 Hz, | |
| | 2H), 0.88 (t, J = 7.3 Hz, 3H). | |
| 169 | ¹H NMR (400 MHz, DMSO-d6) δ 8.35 (s, 2H), 7.99-7.89 (m, | 558.3 |
| | 3H), 7.57 (s, 1H), 7.38 (t, J = 8.0 Hz, 1H), 6.97 (d, J = 8.0 Hz, | |
| | 1H), 6.35-6.14 (m, 2H), 5.37 (d, J = 6.6 Hz, 1H), 5.32 (dd, J = | |
| | 17.3, 1.9 Hz, 1H), 5.21 (d, J = 5.4 Hz, 1H), 5.17-5.03 (m, | |
| | 4H), 4.83 (dd, J = 6.1, 4.1 Hz, 1H), 3.83 (s, 2H), 3.34 (d, J = | |
| | 7.7 Hz, 2H), 3.18 (s, 3H), 3.12 (d, J = 7.7 Hz, 2H). | |
| 170 | ¹H NMR (400 MHz, DMSO-d6) δ 8.35 (s, 2H), 8.02-7.88 (m, | 558.3 |
| | 3H), 7.57 (s, 1H), 7.38 (t, J = 8.0 Hz, 1H), 6.97 (d, J = 7.9 Hz, | |
| | 1H), 6.36-6.15 (m, 2H), 5.37 (d, J = 6.7 Hz, 1H), 5.32 (dd, J = | |
| | 17.3, 1.9 Hz, 1H), 5.21 (d, J = 6.0 Hz, 1H), 5.10 (ddd, J = 12.6, | |
| | 11.2, 6.0 Hz, 4H), 4.83 (dd, J = 6.1, 4.0 Hz, 1H), 3.83 (s, 2H), | |
| | 3.34 (d, J = 7.7 Hz, 2H), 3.18 (s, 3H), 3.12 (d, J = 7.7 Hz, 2H). | |
| 171 | ¹H NMR (400 MHz, DMSO-d6) δ 8.34 (d, J = 9.5 Hz, 2H), 7.97- | 574.3 |
| | 7.86 (m, 3H), 7.57 (s, 1H), 7.38 (t, J = 8.0 Hz, 1H), 6.97 (d, | |
| | J = 7.9 Hz, 1H), 6.28 (d, J = 45.8 Hz, 1H), 5.37 (d, J = 6.7 Hz, | |
| | 1H), 5.21 (d, J = 6.0 Hz, 1H), 5.16-5.02 (m, 3H), 4.85-4.80 | |
| | (m, 1H), 3.79 (s, 2H), 3.24 (d, J = 7.6 Hz, 2H), 3.18 (s, 3H), | |
| | 2.94 (d, J = 7.5 Hz, 2H), 1.66-1.58 (m, 2H), 1.43-1.31 (m, | |
| | 2H), 0.90 (t, J = 7.3 Hz, 3H). | |
| 172 | ¹H NMR (400 MHz, DMSO-d6) δ 8.36 (d, J = 7.5 Hz, 2H), 7.95- | 574.3 |
| | 7.85 (m, 3H), 7.57 (s, 1H), 7.37 (t, J = 8.0 Hz, 1H), 6.97 (d, | |
| | J = 7.9 Hz, 1H), 6.27 (d, J = 45.8 Hz, 1H), 5.37 (d, J = 6.8 Hz, | |
| | 1H), 5.21 (d, J = 5.4 Hz, 1H), 5.11 (t, J = 7.7 Hz, 3H), 4.83 | |
| | (dd, J = 6.2, 4.0 Hz, 1H), 3.79 (s, 2H), 3.24 (d, J = 7.8 Hz, | |
| | 2H), 3.18 (s, 3H), 2.94 (d, J = 7.6 Hz, 2H), 1.62 (dd, J = 10.1, | |
| | 6.1 Hz, 2H), 1.42-1.31 (m, 2H), 0.90 (t, J = 7.4 Hz, 3H). | |
| 173 | ¹H NMR (400 MHz, DMSO-d6) δ 8.35 (s, 2H), 7.98-7.85 (m, | 572.3 |
| | 3H), 7.57 (s, 1H), 7.38 (t, J = 8.0 Hz, 1H), 6.97 (d, J = 7.7 Hz, | |
| | 1H), 6.28 (d, J = 45.8 Hz, 1H), 5.37 (d, J = 6.4 Hz, 1H), 5.21 | |
| | (d, J = 6.4 Hz, 1H), 5.16-5.04 (m, 3H), 4.87-4.78 (m, 1H), | |
| | 3.79 (s, 2H), 3.18 (t, J = 3.8 Hz, 5H), 2.95 (d, J = 7.7 Hz, 2H), | |
| | 1.21-1.13 (m, 1H), 0.37 (t, J = 5.8 Hz, 4H). | |
| 174 | ¹H NMR (400 MHz, DMSO-d6) δ 8.35 (s, 2H), 7.98-7.84 (m, | 572.3 |
| | 3H), 7.57 (s, 1H), 7.38 (t, J = 8.0 Hz, 1H), 6.97 (d, J = 7.6 Hz, | |
| | 1H), 6.28 (d, J = 45.8 Hz, 1H), 5.37 (d, J = 6.7 Hz, 1H), 5.21 | |
| | (d, J = 6.2 Hz, 1H), 5.16-5.03 (m, 3H), 4.86-4.79 (m, 1H), | |
| | 3.79 (s, 2H), 3.18 (s, 5H), 2.95 (d, J = 7.6 Hz, 2H), 1.17 (t, J = | |
| | 5.7 Hz, 1H), 0.37 (d, J = 8.1 Hz, 4H). | |
| 175 | ¹H NMR (400 MHz, DMSO-d6) δ 8.35 (s, 1H), 8.03 (s, 1H), | 558.2 |
| | 7.98 (s, 1H), 7.93 (d, J = 8.1 Hz, 1H), 7.55 (s, 1H), 7.38 (t, J = | |
| | 8.0 Hz, 1H), 6.97 (d, J = 7.7 Hz, 1H), 6.28 (d, J = 45.8 Hz, 1H), | |
| | 5.37 (d, J = 6.7 Hz, 1H), 5.21 (d, J = 6.0 Hz, 1H), 5.18-5.03 (m, | |
| | 3H), 4.89-4.79 (m, 3H), 4.54 (d, J = 7.3 Hz, 2H), 4.02 (s, 2H), | |
| | 3.18 (s, 3H), 3.01 (t, J = 6.8 Hz, 2H), 2.34 (t, J = 6.8 Hz, 2H). | |
| 176 | ¹H NMR (400 MHz, DMSO-d6) δ 8.35 (s, 1H), 8.03 (s, 1H), | 558.3 |
| | 7.98 (s, 1H), 7.93 (d, J = 8.1 Hz, 1H), 7.55 (s, 1H), 7.38 (t, J = | |
| | 8.0 Hz, 1H), 6.97 (d, J = 7.7 Hz, 1H), 6.28 (d, J = 45.8 Hz, 1H), | |
| | 5.37 (d, J = 6.7 Hz, 1H), 5.21 (d, J = 6.0 Hz, 1H), 5.17-5.03 (m, | |
| | 3H), 4.88-4.79 (m, 3H), 4.54 (d, J = 7.3 Hz, 2H), 4.02 (s, 2H), | |
| | 3.18 (s, 3H), 3.01 (t, J = 6.8 Hz, 2H), 2.34 (t, J = 6.8 Hz, 2H) | |
| 177 | ¹H NMR (400 MHz, DMSO-d6) δ 8.35 (s, 1H), 7.96-7.89 (m, | 560.3 |
| | 2H), 7.88 (s, 1H), 7.56 (s, 1H), 7.37 (t, J = 8.0 Hz, 1H), 6.97 (d, | |
| | J = 7.7 Hz, 1H), 6.27 (d, J = 45.8 Hz, 1H), 5.37 (d, J = 6.7 Hz, | |
| | 1H), 5.21 (d, J = 6.1 Hz, 1H), 5.17-4.99 (m, 3H), 4.83 (dd, J = | |
| | 6.0, 4.1 Hz, 1H), 4.33 (bs, 1H), 3.73 (s, 2H), 3.18 (s, 3H), 2.81 | |
| | (t, J = 6.7 Hz, 2H), 2.47-2.40 (m, 1H), 1.66 (q, J = 6.6 Hz, 2H). | |
| | 4 aliphatic protons buried under DMSO-D6 and water peak. | |
| 178 | ¹H NMR (400 MHz, DMSO-d6) δ 8.35 (s, 1H), 7.98-7.89 (m, | 560.5 |
| | 2H), 7.88 (s, 1H), 7.56 (s, 1H), 7.37 (t, J = 8.0 Hz, 1H), 6.97 (d, | |
| | J = 7.7 Hz, 1H), 6.27 (d, J = 45.8 Hz, 1H), 5.37 (d, J = 6.7 Hz, | |
| | 1H), 5.21 (d, J = 6.0 Hz, 1H), 5.17-4.98 (m, 3H), 4.83 (dd, J = | |
| | 6.0, 4.0 Hz, 1H), 4.36 (bs, 1H), 3.74 (s, 2H), 3.18 (s, 3H), 2.82 | |
| | (t, J = 6.5 Hz, 2H), 2.48-2.41 (m, 1H), 1.66 (q, J = 6.6 Hz, 2H). | |
| | 4 aliphatics protons under DMSO or water peak | |
| 179 | ¹H NMR (400 MHz, DMSO-d6) δ 8.32 (s, 1H), 7.98-7.82 (m, | 560.3 |
| | 3H), 7.54 (s, 1H), 7.35 (t, J = 8.0 Hz, 1H), 6.95 (d, J = 7.7 Hz, | |
| | 1H), 6.25 (d, J = 45.9 Hz, 1H), 5.34 (d, J = 6.7 Hz, 1H), 5.19 | |
| | (d, J = 6.0 Hz, 1H), 5.16-4.99 (m, 3H), 4.81 (dd, J = 6.0, 4.1 | |
| | Hz, 1H), 3.79 (s, 2H), 3.16 (s, 3H), 3.12 (d, J = 7.8 Hz, 2H), | |
| | 3.08 (s, 3H), 3.02 (d, J = 7.5 Hz, 2H), 1.38 (s, 3H). | |
| 180 | ¹H NMR (400 MHz, DMSO-d6) δ 8.35 (s, 1H), 8.00-7.86 (m, | 560.5 |
| | 3H), 7.56 (s, 1H), 7.38 (t, J = 8.0 Hz, 1H), 6.97 (d, J = 7.7 Hz, | |
| | 1H), 6.28 (d, J = 45.8 Hz, 1H), 5.37 (d, J = 6.7 Hz, 1H), 5.21 | |
| | (d, J = 6.1 Hz, 1H), 5.17-5.02 (m, 3H), 4.83 (dd, J = 5.9, 4.1 | |
| | Hz, 1H), 3.81 (s, 2H), 3.18 (s, 3H), 3.14 (d, J = 7.8 Hz, 2H), | |
| | 3.11 (s, J = 14.8 Hz, 3H), 3.04 (d, J = 7.4 Hz, 2H), 1.40 (s, 3H). | |
| 181 | ¹H NMR (400 MHz, DMSO-d6) δ 8.35 (s, 1H), 7.98-7.85 (m, | 606.2 |
| | 3H), 7.56 (s, 1H), 7.38 (t, J = 8.0 Hz, 1H), 6.97 (d, J = 7.7 Hz, | |
| | 1H), 6.28 (d, J = 45.9 Hz, 1H), 5.37 (d, J = 6.7 Hz, 1H), 5.21 | |
| | (d, J = 6.1 Hz, 1H), 5.19-5.02 (m, 3H), 4.83 (dd, J = 6.0, 4.1 | |
| | Hz, 1H), 4.36 (s, 4H), 3.79 (s, 2H), 3.41 (s, 4H), 3.18 (s, 3H). | |
| 182 | ¹H NMR (400 MHz, DMSO-d6) δ 8.35 (s, 1H), 8.02-7.85 (m, | 606.5 |
| | 3H), 7.56 (s, 1H), 7.38 (t, J = 8.0 Hz, 1H), 6.97 (d, J = 7.7 Hz, | |
| | 1H), 6.28 (d, J = 45.9 Hz, 1H), 5.37 (d, J = 6.7 Hz, 1H), 5.21 | |
| | (d, J = 6.0 Hz, 1H), 5.19-5.03 (m, 3H), 4.88-4.78 (m, 1H), | |
| | 4.36 (s, 4H), 3.79 (s, 2H), 3.41 (s, 4H), 3.18 (s, 3H). | |
| 183 | ¹H NMR (400 MHz, DMSO-d6) δ 8.35 (s, 1H), 8.22 (s, 1H), | 574.4 |
| | 7.96-7.86 (m, 3H), 7.57 (s, 1H), 7.38 (t, J = 8.0 Hz, 1H), 6.97 | |
| | (d, J = 7.9 Hz, 1H), 6.28 (d, J = 45.8 Hz, 1H), 5.37 (d, J = 6.7 | |
| | Hz, 1H), 5.21 (d, J = 6.1 Hz, 1H), 5.12 (dd, J = 16.3, 9.4 Hz, | |
| | 3H), 4.88-4.77 (m, 1H), 3.81 (s, 2H), 3.29 (d, J = 7.9 Hz, | |
| | 2H), 3.18 (s, 3H), 2.96 (d, J = 7.8 Hz, 2H), 1.84 (dt, J = 13.3, | |
| | 6.6 Hz, 1H), 0.86 (d, J = 6.8 Hz, 6H). | |
| 184 | ¹H NMR (400 MHz, DMSO-d6) δ 8.35 (s, 1H), 8.24 (s, 1H), | 574.4 |
| | 7.95-7.87 (m, 3H), 7.57 (s, 1H), 7.38 (t, J = 8.0 Hz, 1H), 6.97 | |
| | (d, J = 7.7 Hz, 1H), 6.27 (d, J = 45.8 Hz, 1H), 5.37 (d, J = 6.7 | |
| | Hz, 1H), 5.21 (d, J = 5.6 Hz, 1H), 5.17-5.04 (m, 3H), 4.83 | |
| | (dd, J = 6.1, 4.0 Hz, 1H), 3.81 (s, 2H), 3.29 (d, J = 8.0 Hz, | |
| | 2H), 3.18 (s, 3H), 2.95 (d, J = 7.9 Hz, 2H), 1.84 (dt, J = 13.5, | |
| | 6.8 Hz, 1H), 0.86 (d, J = 6.8 Hz, 6H). | |
| 185 | ¹H NMR (400 MHz, DMSO-d6) δ 8.35 (s, 1H), 8.25 (s, 1H), | 576.4 |
| | 7.95-7.87 (m, 3H), 7.56 (s, 1H), 7.38 (t, J = 8.0 Hz, 1H), 6.97 | |
| | (d, J = 7.8 Hz, 1H), 6.27 (d, J = 45.8 Hz, 1H), 5.37 (d, J = 6.8 | |
| | Hz, 1H), 5.21 (d, J = 6.1 Hz, 1H), 5.16-5.03 (m, 3H), 4.83 (dd, | |
| | J = 6.1, 4.0 Hz, 1H), 3.80 (s, 2H), 3.45 (s, 2H), 3.32 (s, 3H), | |
| | 3.30 (d, J = 8.0 Hz, 2H), 3.18 (s, 3H), 2.91 (d, J = 7.8 Hz, 2H). | |
| 186 | ¹H NMR (400 MHz, DMSO-d6) δ 8.35 (s, 1H), 8.24 (s, 1H), | 576.4 |
| | 7.95-7.86 (m, 3H), 7.56 (s, 1H), 7.38 (t, J = 8.0 Hz, 1H), 6.97 | |
| | (d, J = 7.7 Hz, 1H), 6.27 (d, J = 45.9 Hz, 1H), 5.37 (d, J = 6.8 | |
| | Hz, 1H), 5.21 (d, J = 5.9 Hz, 1H), 5.17-5.04 (m, 3H), 4.85- | |
| | 4.81 (m, 1H), 3.80 (s, 2H), 3.45 (s, 2H), 3.32 (s, 3H), 3.30 (d, | |
| | J = 8.0 Hz, 2H), 3.18 (s, 3H), 2.91 (d, J = 7.8 Hz, 2H). | |
| 187 | ¹H NMR (400 MHz, CDCl3) δ 8.09 (s, 1H), 7.91 (s, 1H), 7.83 | 601.2 |
| | (s, 1H), 7.64 (d, J = 8.0 Hz, 1H), 7.49 (br s, 1H), 7.38 (t, J = | |
| | 8.0 Hz, 1H), 6.77 (d, J = 8.4 Hz, 1H), 6.47 (d, J = 46 Hz, 1H), | |
| | 5.35-5.21 (m, 3H), 5.04-4.84 (m, 3H), 4.28 (d, J = 14 Hz, | |
| | 1H), 3.25 (d, J = 14 Hz, 1H), 3.04 (s, 3H), 2.81 (d, J = 10.4 | |
| | Hz, 1H), 2.75-2.64 (m, 2H), 2.47-2.03 (m, 8H), 1.00 (d, J = | |
| | 6.8 Hz, 3H), 0.97 (d, J = 6.8 Hz, 3H). | |
| 188 | ¹H NMR (400 MHz, CDCl3) δ 8.08 (s, 1H), 7.91 (s, 1H), 7.84 | 601.2 |
| | (s, 1H), 7.63 (dd, J = 1.6, 8.0 Hz, 1H), 7.49 (br s, 1H), 7.37 (t, | |
| | J = 8.0 Hz, 1H), 6.77 (d, J = 8.0 Hz, 1H), 6.47 (d, J = 46 Hz, | |
| | 1H), 5.34-5.26 (m, 2H), 5.23 (d, J = 6.4 Hz, 1H), 5.02-4.84 | |
| | (m, 3H), 4.27 (d, J = 14 Hz, 1H), 3.24 (d, J = 14 Hz, 1H), 3.03 | |
| | (d, J = 1.6 Hz, 3H), 2.76-2.65 (m, 2H), 2.60 (d, J = 10.8 Hz, | |
| | 1H), 2.37-2.31 (m, 1H), 2.31-2.21 (m, 5H), 2.08 (td, J = 2.8, | |
| | 10.8 Hz, 1H), 2.03-1.96 (m, 1H), 1.00 (d, J = 6.8 Hz, 3H), | |
| | 0.97 (d, J = 7.2 Hz, 3H). | |
| 189 | ¹H NMR (400 MHz, CDCl3) δ 8.09 (s, 1H), 7.88 (s, 1H), 7.84 | 635.1 |
| | (s, 1H), 7.64-7.61 (m, 1H), 7.58 (s, 1H), 7.37 (t, J = 8.0 Hz, | |
| | 1H), 6.82 (d, J = 8.0 Hz, 1H), 6.08 (d, J = 45.2 Hz, 1H), 4.99- | |
| | 4.85 (m, 2H), 4.28 (d, J = 14.0 Hz, 1H), 3.64-3.47 (m, 2H), | |
| | 3.26-3.17 (m, 2H), 3.06-2.92 (m, 1H), 2.89 (d, J = 1.6 Hz, | |
| | 3H), 2.77-2.66 (m, 2H), 2.62-2.59 (m, 1H), 2.36-2.32 (m, | |
| | 1H), 2.29 (s, 3H), 2.28-2.20 (m, 2H), 2.13-1.97 (m, 2H), | |
| | 1.00 (d, J = 6.8 Hz, 3H), 0.97 (d, J = 6.8 Hz, 3H). | |
| 190 | ¹H NMR (400 MHz, CDCl₃) δ 8.09 (s, 1H), 7.88 (s, 1H), 7.84 | 635.1 |
| | (s, 1H), 7.65-7.60 (m, 1H), 7.58 (s, 1H), 7.37 (t, J = 8.0 Hz, | |
| | 1H), 6.84-6.81 (m, 1H), 6.08 (d, J = 45.2 Hz, 1H), 5.01- | |
| | 4.85 (m, 2H), 4.28 (d, J = 14.0 Hz, 1H), 3.73-3.44 (m, 2H), | |
| | 3.26-3.13 (m, 2H), 3.08-2.92 (m, 1H), 2.89 (d, J = 1.6 Hz, | |
| | 3H), 2.79-2.58 (m, 3H), 2.37-2.34 (m, 1H), 2.30 (s, 3H), | |
| | 2.29-2.21 (m, 2H), 2.15-2.01 (m, 2H), 1.00 (d, J = 6.8 Hz, | |
| | 3H), 0.97 (d, J = 6.8 Hz, 3H). | |
| 191 | ¹H NMR (400 MHz, CDCl3) δ 8.09 (s, 1H), 7.93 (s, 1H), 7.91 | 490.2 |
| | (s, 1H), 7.67 (dd, J = 1.6, 8.4 Hz, 1H), 7.45 (br s, 1H), 7.38 (t, | |
| | J = 8.0 Hz, 1H), 6.78 (d, J = 8.0 Hz, 1H), 6.47 (d, J = 46 Hz, | |
| | 1H), 5.34-5.28 (m, 2H), 5.24 (d, J = 7.2 Hz, 1H), 5.02-4.85 | |
| | (m, 3H), 4.35 (q, J = 6.8 Hz, 1H), 3.04 (d, J = 1.2 Hz, 3H), | |
| | 1.45 (d, J = 6.4 Hz, 3H). | |
| 192 | ¹H NMR (400 MHz, CDCl3) δ 8.09 (s, 1H), 7.92 (s, 1H), 7.91 | 490.0 |
| | (s, 1H), 7.67 (dd, J = 1.2, 8.0 Hz, 1H), 7.45 (br s, 1H), 7.38 (t, | |
| | J = 8.0 Hz, 1H), 6.78 (d, J = 8.0 Hz, 1H), 6.47 (d, J = 46 Hz, | |
| | 1H), 5.34-5.28 (m, 2H), 5.24 (d, J = 6.4 Hz, 1H), 5.02-4.85 | |
| | (m, 3H), 4.35 (q, J = 6.4 Hz, 1H), 3.04 (d, J = 1.2 Hz, 3H), | |
| | 1.45 (d, J = 6.8 Hz, 3H). | |
| 193 | ¹H NMR (400 MHz, CDCl3) δ 8.08 (s, 1H), 7.91 (s, 1H), 7.84 | 629.2 |
| | (d, J = 4.4 Hz, 1H), 7.64 (d, J = 8.0 Hz, 1H), 7.47 (d, J = 7.6 | |
| | Hz, 1H), 7.39 (t, J = 8.0 Hz, 1H), 6.79 (t, J = 7.2 Hz, 1H), 6.47 | |
| | (d, J = 45.6 Hz, 1H), 5.36-5.20 (m, 3H), 5.04-4.86 (m, 3H), | |
| | 4.33-4.10 (m, 2H), 3.36 (d, J = 14.4 Hz, 1H), 3.27-3.13 (m, | |
| | 1H), 3.04 (d, J = 3.6 Hz, 3H), 3.00-2.86 (m, 1H), 2.81-2.69 | |
| | (m, 1H), 2..31-2.15 (m, 3H), 2.09 (d, J = 13.2 Hz, 3H), 1.06 | |
| | (d, J = 6.4 Hz, 3H), 1.00 (d, J = 5.2 Hz, 3H). | |
| 194 | ¹H NMR (400 MHz, CDCl3) δ 8.10 (s, 1H), 7.93 (s, 1H), 7.85 | 629.1 |
| | (s, 1H), 7.65 (d, J = 8.0 Hz, 1H), 7.48 (d, J = 6.0 Hz, 1H), 7.40 | |
| | (dt, J = 2.4, 8.0 Hz, 1H), 6.81 (t, J = 8.0 Hz, 1H), 6.48 (d, J = | |
| | 45.6 Hz, 1H), 5.38-5.20 (m, 3H), 5.06-4.86 (m, 3H), 4.35- | |
| | 4.10 (m, 2H), 3.72-3.51 (m, 1H), 3.38 (d, J = 14 Hz, 1H), | |
| | 3.29-3.13 (m, 1H), 3.10-3.02 (m, 3H), 3.01-2.89 (m, 1H), | |
| | 2.83-2.70 (m, 1H), 2.33-2.14 (m, 3H), 2.14-2.05 (d, J = | |
| | 13.2 Hz, 3H), 1.08 (d, J = 6.4 Hz, 3H), 1.05-0.95 (m, 3H). | |
| 195 | ¹H NMR (400 MHz, CDCl3) δ 8.08 (s, 1H), 7.91 (s, 1H), 7.84 | 665.1 |
| | (s, 1H), 7.63 (dd, J = 1.6, 8.0 Hz, 1H), 7.49 (br s, 1H), 7.37 (t, | |
| | J = 8.0 Hz, 1H), 6.77 (d, J = 8.0 Hz, 1H), 6.47 (d, J = 46 Hz, | |
| | 1H), 5.34-5.26 (m, 2H), 5.23 (d, J = 6.4 Hz, 1H), 5.02-4.84 | |
| | (m, 3H), 4.27 (d, J = 14 Hz, 1H), 3.24 (d, J = 14 Hz, 1H), 3.03 | |
| | (d, J = 1.6 Hz, 3H), 2.76-2.65 (m, 2H), 2.60 (d, J = 10.8 Hz, | |
| | 1H), 2.37-2.31 (m, 1H), 2.31-2.21 (m, 5H), 2.08 (td, J = 2.8, | |
| | 10.8 Hz, 1H), 2.03-1.96 (m, 1H), 1.00 (d, J = 6.8 Hz, 3H), | |
| | 0.97 (d, J = 7.2 Hz, 3H). | |
| 196 | ¹H NMR (400 MHz, CDCl3) δ 8.09 (s, 1H), 7.91 (s, 1H), 7.82 | 665.1 |
| | (s, 1H), 7.63 (dd, J = 1.6, 8.0 Hz, 1H), 7.47 (br s, 1H), 7.39 (t, | |
| | J = 8.0 Hz, 1H), 6.80 (d, J = 7.6 Hz, 1H), 6.47 (d, J = 46 Hz, | |
| | 1H), 5.34-5.21 (m, 3H), 5.04-4.86 (m, 3H), 4.29 (d, J = 14 | |
| | Hz, 1H), 3.57 (d, J = 11.6 Hz, 1H), 3.49-3.41 (m, 1H), 3.38 | |
| | (d, J = 14 Hz, 1H), 3.05 (d, J = 1.2 Hz, 3H), 2.92-2.76 (m, | |
| | 6H), 2.45-2.30 (m, 3H), 1.05 (d, J = 6.8 Hz, 3H), 0.99 (d, J = | |
| | 6.8 Hz, 3H). | |
| 197 | ¹H NMR (400 MHz, CDCl3) δ 8.08 (s, 1H), 7.91 (s, 1H), 7.83 | 633.1 |
| | (s, 1H), 7.64 (dd, J = 1.6, 8.0 Hz, 1H), 7.48 (br s, 1H), 7.38 (t, | |
| | J = 8.0 Hz, 1H), 6.77 (d, J = 8.0 Hz, 1H), 6.47 (d, J = 46 Hz, | |
| | 1H), 5.33-5.27 (m, 2H), 5.23 (d, J = 6.8 Hz, 1H), 5.02-4.85 | |
| | (m, 3H), 4.66 (t, J = 4.4 Hz, 1H), 4.54 (t, J = 4.8 Hz, 1H), 4.27 | |
| | (d, J = 14.4 Hz, 1H), 3.26 (d, J = 14 Hz, 1H), 3.03 (d, J = 1.2 | |
| | Hz, 3H), 2.88 (d, J = 11.2 Hz, 1H), 2.84-2.65 (m, 4H), 2.45- | |
| | 2.13 (m, 9H), 1.00 (d, J = 6.8 Hz, 3H), 0.96 (d, J = 7.2 Hz, 3H). | |
| 198 | ¹H NMR (400 MHz, CDCl3) δ 8.08 (s, 1H), 7.91 (s, 1H), 7.83 | 633.3 |
| | (s, 1H), 7.63 (dd, J = 1.6, 8.0 Hz, 1H), 7.48 (s, 1H), 7.38 (t, J = | |
| | 8.0 Hz, 1H), 6.78 (d, J = 7.6 Hz, 1H), 6.47 (d, J = 46 Hz, | |
| | 1H), 5.34-5.27 (m, 2H), 5.26-5.22 (m, 1H), 5.02-4.84 (m, | |
| | 3H), 4.66 (br s, 1H), 4.54 (br s, 1H), 4.28 (d, J = 14.4 Hz, 1H), | |
| | 3.26 (d, J = 14 Hz, 1H), 3.04 (d, J = 1.6 Hz, 3H), 2.93-2.55 | |
| | (m, 5H), 2.45-2.10 (m, 5H), 1.00 (d, J = 6.8 Hz, 3H), 0.97 (d, | |
| | J = 6.8 Hz, 3H). | |
| 199 | ¹H NMR (400 MHz, CDCl3) δ 8.08 (s, 1H), 7.91 (s, 1H), 7.83 | 643.3 |
| | (s, 1H), 7.63 (br d, J = 7.6 Hz, 1H), 7.48 (s, 1H), 7.38 (t, J = | |
| | 8.0 Hz, 1H), 6.78 (d, J = 7.2 Hz, 1H), 6.47 (d, J = 46 Hz, 1H), | |
| | 5.34-5.22 (m, 3H), 5.01-4.86 (m, 3H), 4.71-4.59 (m, 4H), | |
| | 4.27 (d, J = 14.6 Hz, 1H), 3.53-3.43 (m, 1 1H), 3.29 (d, J = | |
| | 14 Hz, 1H), 3.04 (d, J = 1.2 Hz, 3H), 2.76-2.70 (m, 1H), 2.62 | |
| | (br d, J = 10.8 Hz, 1H), 2.55-2.49 (br s, 1H), 2.39-2.23 (m, | |
| | 3H), 2.05-1.99 (m, 1H), 1.93 (t, J = 6.4 Hz, 1H), 0.99 (d, J = | |
| | 6.8 Hz, 3H), 0.95 (d, J = 6.8 Hz, 3H). | |
| 200 | ¹H NMR (400 MHz, CDCl3) δ 8.08 (s, 1H), 7.90 (s, 1H), 7.83 | 643.2 |
| | (s, 1H), 7.64 (d, J = 8.0 Hz, 1H), 7.48 (s, 1H), 7.38 (t, J = 8.0 | |
| | Hz, 1H), 6.78 (d, J = 7.2 Hz, 1H), 6.47 (d, J = 46 Hz, 1H), 5.36- | |
| | 5.27 (m, 2H), 5.23 (d, J = 6.8 Hz, 1H), 5.03-4.85 (m, 3H), | |
| | 4.72-4.58 (m, 4H), 4.27 (d, J = 14 Hz, 1H), 3.55-3.43 (m, | |
| | 1H), 3.29 (d, J = 14 Hz, 1H), 3.04 (s, 3H), 2.73 (d, J = 11.6 | |
| | Hz, 1H), 2.62 (d, J = 10.8 Hz, 1H), 2.52 (t, J = 11.2 Hz, 1H), | |
| | 2.42-2.24 (m, 3H), 2.02 (dt, J = 2.4, 10.4 Hz, 1H), 1.93 (t, J = | |
| | 10.4 Hz, 1H), 0.99 (d, J = 6.8 Hz, 3H), 0.95 (d, J = 6.8 Hz, 3H). | |
| 201 | ¹H NMR (400 MHz, CDCl3) δ 8.08 (s, 1H), 7.91 (s, 1H), 7.83 | 669.3 |
| | (s, 1H), 7.64 (dd, J = 1.6, 8.0 Hz, 1H), 7.48 (s, 1H), 7.38 (t, J = | |
| | 8.0 Hz, 1H), 6.78 (d, J = 7.6 Hz, 1H), 6.47 (d, J = 46.4 Hz, | |
| | 1H), 5.34-5.21 (m, 3H), 5.02-4.85 (m, 3H), 4.25 (d, J = 14 | |
| | Hz, 1H), 3.29 (d, J = 14 Hz, 1H), 3.04 (d, J = 1.6 Hz, 3H), 3.01- | |
| | 2.89 (m, 2H), 2.85 (br d, J = 10 Hz, 1H), 2.78-2.66 (m, 2H), 2.50- | |
| | 2.25 (m, 5H), 0.99 (d, J = 6.8 Hz, 3H), 0.96 (d, J = 6.8 Hz, 3H). | |
| 202 | ¹H NMR (400 MHz, CDCl3) δ 8.07 (s, 1H), 7.90 (s, 1H), 7.83 | 669.2 |
| | (s, 1H), 7.63 (dd, J = 1.6, 8.0 Hz, 2H), 7.48 (br s, 1H), 7.38 (t, | |
| | J = 8.0 Hz, 1H), 6.78 (d, J = 7.6 Hz, 1H), 6.47 (d, J = 46 Hz, | |
| | 1H), 5.35-5.18 (m, 3H), 5.03-4.83 (m, 3H), 4.25 (d, J = 14 Hz, | |
| | 1H), 3.29 (d, J = 14 Hz, 1H), 3.04 (d, J = 1.2 Hz, 3H), 3.01-2.90 | |
| | (m, 2H), 2.85 (d, J = 10 Hz, 1H), 2.79-2.65 (m, 2H), 2.50-2.23 | |
| | (m, 5H), 0.99 (d, J = 6.8 Hz, 3H), 0.95 (d, J = 6.8 Hz, 3H). | |
| 203 | ¹H NMR (400 MHz, DMSO-d6) δ 8.36 (s, 1H), 7.96 (dd, J = | 457.0/459.0 |
| | 8.3, 1.5 Hz, 1H), 7.91 (dd, J = 7.9, 0.6 Hz, 1H), 7.80 (d, J = | (Br pattern) |
| | 7.4 Hz, 1H), 7.60-7.49 (m, 2H), 7.37 (t, J = 8.0 Hz, 1H), 6.96 | |
| | (d, J = 7.8 Hz, 1H), 6.27 (d, J = 45.9 Hz, 1H), 5.37 (d, J = 6.6 | |
| | Hz, 1H), 5.22 (dd, J = 6.1, 1.1 Hz, 1H), 5.13 (dd, J = 6.7, 1.9 | |
| | Hz, 1H), 4.97-4.81 (m, 3H), 3.19 (s, 3H). | |
| 204 | ¹H NMR (400 MHz, DMSO-d6) δ 8.36 (s, 1H), 7.97 (dd, J = | 413.1/415.0 |
| | 7.9, 1.7 Hz, 1H), 7.78 (d, J = 2.7 Hz, 1H), 7.76 (d, J = 2.5 Hz, | (Cl pattern) |
| | 1H), 7.60 (t, J = 7.7 Hz, 1H), 7.55 (s, 1H), 7.37 (t, J = 8.0 Hz, | |
| | 1H), 6.96 (d, J = 8.0 Hz, 1H), 6.27 (d, J = 45.9 Hz, 1H), 5.37 | |
| | (d, J = 6.9 Hz, 1H), 5.22 (d, J = 5.2 Hz, 1H), 5.13 (dd, J = 6.8, | |
| | 1.9 Hz, 1H), 4.97 (q, J = 17.3 Hz, 2H), 4.83 (dd, J = 6.1, 4.0 | |
| | Hz, 1H), 3.19 (s, 3H). | |
| 205 | ¹H NMR (400 MHz, DMSO-d6) δ 8.36 (s, 1H), 8.08 (dd, J = | 505.1 |
| | 7.8, 0.8 Hz, 1H), 7.93 (dd, J = 8.2, 1.5 Hz, 1H), 7.80 (d, J = | |
| | 7.5 Hz, 1H), 7.56 (t, J = 1.8 Hz, 1H), 7.37 (td, J = 7.8, 2.1 Hz, | |
| | 2H), 6.95 (d, J = 8.1 Hz, 1H), 6.28 (d, J = 45.9 Hz, 1H), 5.37 | |
| | (d, J = 6.6 Hz, 1H), 5.22 (d, J = 5.1 Hz, 1H), 5.12 (dd, J = 6.8, | |
| | 1.9 Hz, 1H), 4.91-4.65 (m, 3H), 3.18 (s, 3H). | |
| 206 | ¹H NMR (400 MHz, DMSO-d6) δ 8.35 (s, 1H), 8.04 (d, J = 9.8 | 409.2 |
| | Hz, 1H), 7.53 (t, J = 7.8 Hz, 1H), 7.47 (s, 1H), 7.40-7.24 (m, | |
| | 3H), 6.93 (d, J = 7.9 Hz, 1H), 6.25 (d, J = 45.9 Hz, 1H), 5.36 | |
| | (d, J = 6.5 Hz, 1H), 5.21 (d, J = 6.4 Hz, 1H), 5.13 (d, J = 6.8 | |
| | Hz, 1H), 4.98-4.73 (m, 3H), 3.94 (s, 3H), 3.18 (s, 3H). | |
| 207 | ¹H NMR (400 MHz, DMSO-d6) δ 8.35 (s, 1H), 8.02 (dd, J = | 437.1 |
| | 8.3, 2.1 Hz, 1H), 7.59-7.42 (m, 2H), 7.41-7.22 (m, 3H), | |
| | 6.90 (d, J = 7.7 Hz, 1H), 6.26 (d, J = 45.8 Hz, 1H), 5.37 (d, J = | |
| | 6.7 Hz, 1H), 5.21 (d, J = 5.5 Hz, 1H), 5.13 (d, J = 6.8 Hz, | |
| | 1H), 4.95-4.66 (m, 4H), 3.17 (s, 3H), 1.36 (d, J = 6.0 Hz, 6H). | |
| 208 | ¹H NMR (400 MHz, DMSO-d6) δ 8.36 (s, 1H), 7.95 (d, J = 8.0 | 393.3 |
| | Hz, 1H), 7.68-7.54 (m, 2H), 7.54-7.42 (m, 2H), 7.37 (t, J = | |
| | 8.0 Hz, 1H), 6.93 (d, J = 7.8 Hz, 1H), 6.27 (d, J = 45.9 Hz, 1H), | |
| | 5.37 (d, J = 6.6 Hz, 1H), 5.21 (d, J = 5.7 Hz, 1H), 5.12 (d, | |
| | J = 6.7 Hz, 1H), 4.99-4.79 (m, 3H), 3.18 (s, 3H), 2.41 (s, 3H). | |
| 209 | ¹H NMR (400 MHz, DMSO-d6) δ 8.36 (s, 1H), 7.98-7.92 (m, | 407.3 |
| | 1H), 7.63-7.44 (m, 4H), 7.37 (t, J = 8.0 Hz, 1H), 6.93 (d, J = | |
| | 7.8 Hz, 1H), 6.26 (d, J = 45.9 Hz, 1H), 5.37 (d, J = 6.7 Hz, | |
| | 1H), 5.21 (d, J = 6.1 Hz, 1H), 5.13 (d, J = 6.7 Hz, 1H), 5.03- | |
| | 4.87 (m, 2H), 4.84 (dd, J = 6.1, 4.1 Hz, 1H), 3.17 (s, 3H), 2.75 | |
| | (q, J = 7.6 Hz, 2H), 1.28 (t, J = 7.6 Hz, 3H). | |
| 210 | ¹H NMR (400 MHz, DMSO-d6) δ 8.36 (s, 1H), 7.87 (dd, J = | 455.3 |
| | 8.2, 1.4 Hz, 1H), 7.80 (dd, J = 7.4, 1.1 Hz, 1H), 7.75 (dd, J = | |
| | 7.6, 1.2 Hz, 1H), 7.72-7.63 (m, 3H), 7.60-7.52 (m, 3H), | |
| | 7.48 (dd, J = 8.4, 6.3 Hz, 1H), 7.36 (t, J = 8.0 Hz, 1H), 6.94 | |
| | (d, J = 8.1 Hz, 1H), 6.26 (d, J = 45.7 Hz, 1H), 5.35 (d, J = 6.5 | |
| | Hz, 1H), 5.18 (d, J = 5.4 Hz, 1H), 5.10-5.05 (m, 3H), 4.81 | |
| | (dd, J = 6.2, 4.0 Hz, 1H), 3.14 (s, 3H). | |
| 211 | ¹H NMR (400 MHz, DMSO-d6) δ 8.35 (s, 1H), 7.97 (d, J = 9.8 | 403.2 |
| | Hz, 1H), 7.87-7.72 (m, 2H), 7.66-7.49 (m, 2H), 7.36 (t, J = | |
| | 8.0 Hz, 1H), 6.94 (d, J = 7.8 Hz, 1H), 6.27 (d, J = 45.9 Hz, | |
| | 1H), 5.37 (d, J = 6.7 Hz, 1H), 5.22 (d, J = 6.0 Hz, 1H), 5.12 | |
| | (d, J = 6.8 Hz, 1H), 5.08-4.91 (m, 2H), 4.89-4.80 (m, 1H), | |
| | 4.69 (s, 1H), 3.18 (s, 3H). | |
| 212 | ¹H NMR (400 MHz, DMSO-d6) δ 8.32 (s, 1H), 8.22 (s, 1H), | 490.3 |
| | 7.99 (d, J = 16.8 Hz, 2H), 7.91 (dd, J = 8.3, 1.4 Hz, 1H), 7.53 | |
| | (s, 1H), 7.35 (t, J = 8.0 Hz, 1H), 6.95 (d, J = 8.1 Hz, 1H), 6.25 | |
| | (d, J = 45.9 Hz, 1H), 5.34 (d, J = 6.7 Hz, 1H), 5.19 (d, J = 6.0 | |
| | Hz, 1H), 5.14-5.02 (m, 3H), 4.80 (dd, J = 6.1, 4.0 Hz, 1H), | |
| | 3.86 (s, 2H), 3.15 (d, J = 7.3 Hz, 3H), 2.27 (s, 3H). | |
| 213 | ¹H NMR (400 MHz, DMSO-d6) δ 8.32 (s, 1H), 8.22 (s, 1H), | 504.3 |
| | 8.00 (d, J = 18.1 Hz, 2H), 7.91 (dd, J = 8.2, 1.4 Hz, 1H), 7.53 | |
| | (s, 1H), 7.35 (t, J = 8.0 Hz, 1H), 6.95 (d, J = 7.9 Hz, 1H), 6.25 | |
| | (d, J = 45.8 Hz, 1H), 5.34 (d, J = 6.7 Hz, 1H), 5.19 (d, J = 6.1 | |
| | Hz, 1H), 5.15-5.02 (m, 3H), 4.80 (dd, J = 5.9, 4.1 Hz, 1H), | |
| | 3.90 (s, 2H), 3.16 (s, 3H), 2.54 (dd, J = 14.2, 7.0 Hz, 2H), 1.03 | |
| | (t, J = 7.1 Hz, 3H). | |
| 214 | ¹H NMR (400 MHz, methanol-d₄) δ = 8.15 (s, 1H), 8.06 (s, | 589.4 |
| | 1H), 7.93 (s, 1H), 7.73 (dd, J = 1.6, 8.0 Hz, 1H), 7.46 (t, J = | |
| | 1.6 Hz, 1H), 7.38 (t, J = 8.0 Hz, 1H), 6.83 (d, J = 8.0 Hz, | |
| | 1H), 5.06 (s, 2H), 4.27 (d, J = 14 Hz, 1H), 3.42-3.33 (m, | |
| | 4H), 3.29-3.24 (m, 1H), 3.11-2.97 (m, 2H), 2.80-2.71 (m, 4H), | |
| | 2.71-2.60 (m, 2H), 2.60-.2.46 (m, 1H), 2.33-2.21 (m, 4H), | |
| | 2.21-2.11 (m, 1H), 2.08-1.96 (m, 1H), 1.17 (d, J = 6.4 Hz, 3H). | |
| 215 | ¹H NMR (400 MHz, CDCl₃) δ 8.09 (s, 1H), 7.86 (s, 1H), 7.84 | 617.3 |
| | (s, 1H), 7.64 (t, J = 1.6 Hz, 1H), 7.50 (dd, J = 1.6, 8.0 Hz, 1H), | |
| | 7.32 (t, J = 8.0 Hz, 1H), 6.68 (d, J = 8.4 Hz, 1H), 4.92 (s, 2H), | |
| | 4.28 (d, J = 14 Hz, 1H), 3.45-3.33 (m, 2H), 3.30 (s, 2H), 3.24 | |
| | (d, J = 14 Hz, 1H), 3.10-2.94 (m, 2H), 2.76 (s, 3H), 2.74- | |
| | 2.66 (m, 1H), 2.64-2.57 (m, 1H), 2.38-2.32 (m, 1H), 2.32- | |
| | 2.21 (m, 5H), 2.09 (td, J = 2.4, 10.8 Hz, 1H), 2.00 (t, J = 10.8 | |
| | Hz, 1H), 1.00 (d, J = 6.8 Hz, 3H), 0.97 (d, J = 7.2 Hz, 3H). | |
| 216 | ¹H NMR (400 MHz, CDCl₃) δ 8.08 (s, 1H), 7.89-7.80 (m, 2H), | 585.2 |
| | 7.65 (d, J = 8.8 Hz, 1H), 7.37-7.29 (m, 2H), 6.76 (d, J = 7.6 | |
| | Hz, 1H), 6.22 (d, J = 44.8 Hz, 1H), 5.00-4.80 (m, 2H), 4.30 | |
| | (d, J = 14 Hz, 1H), 3.24 (d, J = 14 Hz, 1H), 2.99 (d, J = 11.2 | |
| | Hz, 1H), 2.95-2.78 (m, 4H), 2.76 (s, 3H), 2.73-2.55 (m, 3H), | |
| | 2.49-2.10 (m, 5H), 2.07-1.97 (m, 1H), 1.05-0.90 (m, 6H). | |
| 217 | ¹H NMR (400 MHz, CDCl₃) δ 8.09 (s, 1H), 7.88-7.80 (m, 2H), | 585.2 |
| | 7.65 (dd, J = 1.6, 8.0 Hz, 1H), 7.37-7.29 (m, 2H), 6.76 (d, J = | |
| | 7.6 Hz, 1H), 6.22 (d, J = 44.8 Hz, 1H), 4.99-4.81 (m, 2H), | |
| | 4.30 (d, J = 14 Hz, 1H), 3.24 (d, J = 14.4 Hz, 1H), 3.00 (d, J = | |
| | 11.2 Hz, 1H), 2.96-2.78 (m, 4H), 2.76 (d, J = 1.6 Hz, 3H), | |
| | 2.73-2.56 (m, 3H), 2.48-2.10 (m, 5H), 2.08-1.94 (m, 1H), | |
| | 1.05-0.92 (m, 6H). | |
| 218 | ¹H NMR (400 MHz, CDCl₃) δ 8.06 (s, 1H), 7.91 (s, 1H), 7.85 | 601.6 |
| | (s, 1H), 7.66 (dd, J = 1.6, 8.0 Hz, 1H), 7.45 (s, 1H), 7.38 (t, J = | |
| | 8.0 Hz, 1H), 6.79 (d, J = 7.6 Hz, 1H), 6.47 (d, J = 46 Hz, | |
| | 1H), 5.35-5.27 (m, 2H), 5.26-5.20 (m, 1H), 5.04-4.83 (m, | |
| | 3H), 3.74 (d, J = 13.6 Hz, 1H), 3.62 (d, J = 14 Hz, 1H), 3.05 | |
| | (d, J = 1.2 Hz, 1H), 3.03-2.96 (m, 1H), 2.77 (d, J = 11.2 Hz, | |
| | 1H), 2.74-2.67 (m, 1H), 2.54-2.45 (m, 1H), 2.39 (s, 3H), | |
| | 2.38-2.32 (m, 1H), 2.28-2.17 (m, 3H), 0.98-0.88 (m, 6H). | |
| 219 | ¹H NMR (400 MHz, CD₃CN) δ 7.99 (s, 1H), 7.92-7.87 (m, | 603.6 |
| | 2H), 7.86 (s, 1H), 7.37-7.30 (m, 2H), 6.80 (d, J = 7.6 Hz, 1H), | |
| | 4.94 (s, 2H), 4.28 (d, J = 14.4 Hz, 1H), 3.38-3.26 (m, 3H), | |
| | 3.24 (m, 2H), 3.24 (s, 2H), 3.11-2.94 (m, 2H), 2.94-2.85 (m, | |
| | 1H), 2.79-2.70 (m, 1H), 2.68 (s, 3H), 2.67-2.49 (m, 3H), | |
| | 2.37-2.23 (m, 1H), 2.11-2.05 (m, 2H), 0.96 (d, J = 7.2 Hz, | |
| | 3H), 0.92 (d, J = 7.2 Hz, 3H). | |
| 220 | ¹H NMR (400 MHz, CDCl₃) δ 8.07 (s, 1H), 7.86 (s, 1H), 7.82 | 589.2 |
| | (s, 1H), 7.55 (s, 1H), 7.52 (dd, J = 1.2, 8.4 Hz, 1H), 7.33 (t, J = | |
| | 8.0 Hz, 1H), 6.74 (d, J = 8.0 Hz, 1H), 4.92 (s, 2H), 3.73 (s, | |
| | 2H), 3.46-3.33(m, 2H), 3.30 (s, 3H), 3.22-2.93 (m, 4H), 2.92- | |
| | 2.67 (m, 9H), 2.59 (br s, 2H), 1.31 (br s, 3H). | |
| 221 | ¹H NMR (400 MHz, DMSO-d6) δ 8.50 (s, 1H), 7.99 (s, 1H), | 603.2 |
| | 7.92 (s, 1H), 7.89 (s, 1H), 7.83 (dd, J = 7.9, 1.7 Hz, 1H), 7.37 | |
| | (t, J = 7.9 Hz, 1H), 7.16 (d, J = 7.8 Hz, 1H), 5.11 (s, 2H), 4.22 | |
| | (d, J = 14.4 Hz, 1H), 3.40-3.35 (m, 4H), 2.66-2.57 (m, 2H), | |
| | 2.31-2.19 (m, 3H), 2.14 (s, 3H), 1.98-1.89 (m, 2H), 1.45- | |
| | 1.41 (m, 2H), 1.17-1.13 (m, 2H), 0.93 (d, J = 6.7 Hz, 3H), | |
| | 0.88 (d, J = 6.6 Hz, 3H). | |
| 222 | ¹H NMR (400 MHz, DMSO-d6) δ 7.97 (s, 1H), 7.90 (t, J = 4.8 | 583.2 |
| | Hz, 2H), 7.30 (t, J = 8.0 Hz, 1H), 7.26-7.16 (m, 2H), 6.59 (d, | |
| | J = 7.7 Hz, 1H), 5.10-4.97 (m, 4H), 4.94-4.80 (m, 4H), 4.20 | |
| | (d, J = 14.3 Hz, 1H), 3.36 (d, J = 14.3 Hz, 1H), 2.60 (t, J = 10.3 | |
| | Hz, 2H), 2.33-2.15 (m, 3H), 2.13 (s, 3H), 2.00-1.80 (m, 2H), | |
| | 1.47 (s, 3H), 0.91 (d, J = 6.6 Hz, 3H), 0.86 (d, J = 6.6 Hz, 3H). | |
| 226 | ¹H NMR (400 MHz, DMSO-d6) δ 8.35 (s, 1H), 8.23 (s, 1H), | 518.2 |
| | 8.07 (s, 1H), 8.02 (s, 1H), 7.94 (dd, J = 8.2, 1.4 Hz, 1H), 7.55 | |
| | (t, J = 1.8 Hz, 1H), 7.38 (t, J = 8.0 Hz, 1H), 6.97 (d, J = 8.1 | |
| | Hz, 1H), 6.28 (d, J = 45.9 Hz, 1H), 5.37 (d, J = 6.5 Hz, 1H), | |
| | 5.22 (dd, J = 6.2, 0.9 Hz, 1H), 5.18-5.04 (m, 3H), 4.83 (dd, J = | |
| | 6.2, 4.0 Hz, 1H), 3.94 (s, 2H), 3.19 (s, 3H), 2.76 (dt, J = 12.5, | |
| | 6.2 Hz, 1H), 2.66 (q, J = 7.2 Hz, 1H), 1.04 (d, J = 6.3 Hz, 6H). | |
| 227 | ¹H NMR (400 MHz, DMSO-d6) δ 8.35 (s, 1H), 8.23 (s, 1H), | 530.3 |
| | 8.00 (d, J = 15.0 Hz, 2H), 7.94 (dd, J = 8.2, 1.4 Hz, 1H), 7.55 | |
| | (t, J = 1.8 Hz, 1H), 7.38 (t, J = 8.0 Hz, 1H), 6.97 (d, J = 7.9 | |
| | Hz, 1H), 6.28 (d, J = 45.8 Hz, 1H), 5.37 (d, J = 6.5 Hz, 1H), | |
| | 5.21 (dd, J = 6.2, 0.9 Hz, 1H), 5.16-5.03 (m, 3H), 4.83 (dd, | |
| | J = 6.2, 4.0 Hz, 1H), 3.82 (s, 2H), 3.21-3.12 (m, 4H), 2.09- | |
| | 2.01 (m, 2H), 1.76-1.46 (m, 5H). | |
| 228 | ¹H NMR (400 MHz, DMSO-d6) δ 8.35 (s, 1H), 8.27 (s, 1H), | 518.3 |
| | 8.04 (s, 1H), 8.00 (s, 1H), 7.94 (dd, J = 8.2, 1.4 Hz, 1H), 7.56 | |
| | (t, J = 1.8 Hz, 1H), 7.38 (t, J = 8.0 Hz, 1H), 6.97 (d, J = 7.7 | |
| | Hz, 1H), 6.28 (d, J = 45.9 Hz, 1H), 5.37 (d, J = 6.4 Hz, 1H), | |
| | 5.22 (d, J = 5.3 Hz, 1H), 5.17-5.04 (m, 3H), 4.83 (dd, J = 6.2, | |
| | 4.0 Hz, 1H), 3.91 (s, 2H), 3.18 (s, 3H), 2.47 (t, J = 7.1 Hz, | |
| | 2H), 1.50-1.40 (m, 2H), 0.87 (t, J = 7.4 Hz, 3H). | |
| 229 | ¹H NMR (400 MHz, DMSO-d6) δ 8.35 (s, 1H), 8.00 (s, 1H), | 530.3 |
| | 7.97-7.91 (m, 2H), 7.55 (t, J = 1.7 Hz, 1H), 7.37 (t, J = 8.0 | |
| | Hz, 1H), 6.97 (d, J = 7.7 Hz, 1H), 6.27 (d, J = 45.9 Hz, 1H), | |
| | 5.37 (d, J = 6.6 Hz, 1H), 5.21 (dd, J = 6.0, 0.5 Hz, 1H), 5.16-5.02 | |
| | (m, 3H), 4.83 (dd, J = 6.2, 3.9 Hz, 1H), 3.92 (s, 2H), 3.19 (s, 3H), | |
| | 1.25 (s, 3H), 0.51 (q, J = 4.0 Hz, 2H), 0.32 (q, J = 4.0 Hz, 2H). | |
| 230 | ¹H NMR (400 MHz, DMSO-d6) δ 8.35 (s, 1H), 8.25 (s, 1H), | 544.3 |
| | 8.04 (d, J = 18.1 Hz, 2H), 7.95 (dd, J = 8.1, 1.5 Hz, 1H), 7.55 | |
| | (t, J = 1.8 Hz, 1H), 7.38 (t, J = 8.0 Hz, 1H), 6.97 (d, J = 7.8 | |
| | Hz, 1H), 6.28 (d, J = 45.9 Hz, 1H), 5.37 (d, J = 6.5 Hz, 1H), | |
| | 5.22 (d, J = 5.4 Hz, 1H), 5.17-5.03 (m, 3H), 4.83 (dd, J = | |
| | 6.2, 4.0 Hz, 1H), 3.83 (s, 2H), 3.19 (s, 3H), 2.04-1.93 (m, | |
| | 2H), 1.77-1.63 (m, 4H), 1.23 (s, 3H). | |
| 231 | ¹H NMR (400 MHz, DMSO-d6) δ 8.35 (s, 1H), 8.03 (s, 1H), | 536.1 |
| | 7.99 (s, 1H), 7.94 (dd, J = 8.1, 1.8 Hz, 1H), 7.55 (s, 1H), 7.38 | |
| | (t, J = 8.0 Hz, 1H), 6.97 (d, J = 7.7 Hz, 1H), 6.28 (d, J = 45.8 | |
| | Hz, 1H), 5.37 (d, J = 6.7 Hz, 1H), 5.22 (d, J = 6.1 Hz, 1H), | |
| | 5.16-5.03 (m, 3H), 4.83 (dd, J = 6.1, 4.0 Hz, 1H), 4.58 (t, J = | |
| | 6.0 Hz, 1H), 4.46 (t, J = 6.0 Hz, 1H), 3.89 (s, 2H), 3.18 (s, | |
| | 3H), 2.57 (t, J = 6.9 Hz, 2H), 1.89-1.73 (m, 2H). | |
| 275 | ¹H NMR (400 MHz, DMSO-d6) δ 8.18 (s, 1H), 7.95 (s, 1H), | 637.5 |
| | 7.89 (s, 1H), 7.38 (s, 1H), 6.74 (t, J = 5.7 Hz, 1H), 5.85 (m, | |
| | 1H), 5.76 (s, 1H), 5.14 (m, 3H), 5.04 (dd, J = 10.2, 1.4 Hz, | |
| | 1H), 4.19 (d, J = 14.4 Hz, 1H), 3.81 (s, 2H), 3.33 (m, 2H), 3.13 | |
| | (s, 2H), 2.98 (s, 3H), 2.67-2.55 (m, 2H), 2.41 (m, 2H), 2.22 | |
| | (m, 5H), 2.12 (s, 3H), 2.05 (m, 1H), 1.98-1.72 (m, 3H), 0.91 | |
| | (d, J = 6.6 Hz, 3H), 0.86 (d, J = 6.6 Hz, 3H). | |
| 276 | ¹H NMR (400 MHz, DMSO-d6) δ 8.24 (d, J = 6.2 Hz, 1H), 8.18 | 578.4 |
| | (s, 1H), 7.87 (d, J = 8.3 Hz, 1H), 7.41-7.22 (m, 2H), 6.80 (d, | |
| | J = 8.0 Hz, 1H), 5.13 (s, 2H), 3.79 (d, J = 7.9 Hz, 2H), 3.28- | |
| | 3.17 (m, 4H), 3.11-2.91 (m, 2H), 2.72 (s, 3H), 2.05-1.87 | |
| | (m, 2H), 1.77-1.59 (m, 4H), 1.24 (s, 3H). | |
| 277 | ¹H NMR (400 MHz, DMSO-d6) δ 8.25 (s, 1H), 8.00 (s, 1H), | 556.3 |
| | 7.98 (s, 1H), 7.40 (s, 1H), 6.63 (d, J = 6.1 Hz, 1H), 5.93 (s, 1H), | |
| | 5.15 (s, 2H), 4.90 (d, J = 6.0 Hz, 1H), 4.78 (d, J = 6.0 Hz, 2H), | |
| | 4.04-3.92 (m, 2H), 3.87 (s, 4H), 3.48 (s, 3H), 3.23 (s, 3H), 1.98- | |
| | 1.81 (m, 2H), 1.67 (dd, J = 13.9, 10.6 Hz, 2H), 1.59-1.39 (m, 4H). | |
| 278 | ¹H NMR (400 MHz, DMSO-d6) δ 8.30 (s, 1H), 7.94 (s, 1H), | 669.2 |
| | 7.88 (s, 1H), 7.40 (s, 1H), 6.69 (t, J = 5.7 Hz, 1H), 6.04 (d, J = | |
| | 44.8 Hz, 1H), 5.95 (s, 1H), 5.11 (s, 2H), 4.18 (d, J = 14.4 | |
| | Hz, 1H), 3.35 (m, 2H), 3.14 (s, 3H), 3.05 (m, 2H), 2.83-2.54 | |
| | (m, 4H), 2.45-2.37 (m, 1H), 2.34-2.15 (m, 4H), 2.12 (s, 3H), | |
| | 2.09-1.76 (m, 4H), 1.01 (m, 1H), 0.91 (d, J = 6.7 Hz, 3H), | |
| | 0.86 (d, J = 6.7 Hz, 3H), 0.48-0.34 (m, 2H), 0.26-0.10 (m, 2H). | |
| 279 | ¹H NMR (400 MHz, DMSO-d6) δ 8.30 (s, 1H), 7.95 (s, 1H), | 669.2 |
| | 7.89 (s, 1H), 7.40 (s, 1H), 6.68 (t, J = 5.7 Hz, 1H), 6.05 (d, J = | |
| | 44.8 Hz, 1H), 5.94 (s, 1H), 5.09 (d, J = 19.3 Hz, 2H), 4.19 | |
| | (d, J = 14.4 Hz, 1H), 3.41-3.32 (m, 2H), 3.14 (s, 3H), 3.05 | |
| | (t, J = 6.2 Hz, 2H), 2.83-2.53 (m, 4H), 2.40 (d, J = 10.5 Hz, | |
| | 1H), 2.34-2.15 (m, 4H), 2.12 (s, 3H), 2.09-1.76 (m, 4H), | |
| | 1.07-0.95 (m, 1H), 0.90 (t, J = 8.7 Hz, 3H), 0.86 (d, J = 6.6 | |
| | Hz, 3H), 0.46-0.35 (m, 2H), 0.24-0.12 (m, 2H). | |
| 280 | ¹H NMR (400 MHz, DMSO-d6) δ 8.20 (d, J = 5.2 Hz, 1H), 7.97 | 661.3 |
| | (s, 1H), 7.90 (s, 1H), 7.49 (s, 1H), 5.89 (s, 1H), 5.14 (d, J = 10.9 | |
| | Hz, 2H), 4.19 (d, J = 14.3 Hz, 1H), 3.26-3.18 (m, 5H), 2.97 (s, | |
| | 3H), 2.95 (s, 6H), 2.68-2.58 (m, 2H), 2.36-2.17 (m, 4H), 1.99-1.91 | |
| | (m, 2H), 0.94 (d, J = 6.6 Hz, 6H), 0.83 (d, J = 6.6 Hz, 6H) | |
| 281 | ¹H NMR (400 MHz, DMSO-d6) δ 8.18 (s, 1H), 7.93 (dd, J = | 583.3 |
| | 8.0, 1.4 Hz, 1H), 7.67 (d, J = 8.6 Hz, 2H), 7.38 (s, 1H), 7.34 | |
| | (t, J = 8.0 Hz, 1H), 6.78 (d, J = 7.8 Hz, 1H), 4.93 (s, 2H), 4.15 | |
| | (d, J = 14.1 Hz, 1H), 3.32 (s, 2H), 3.26-3.22 (m, 4H), 3.02 | |
| | (q, J = 14.1 Hz, 2H), 2.72 (s, J = 3.1 Hz, 3H), 2.67-2.55 (m, | |
| | 2H), 2.35-2.16 (m, 3H), 2.14 (s, 3H), 1.97-1.86 (m, 2H), | |
| | 0.93 (d, J = 6.7 Hz, 3H), 0.89 (d, J = 6.7 Hz, 3H). | |
| 282 | ¹H NMR (400 MHz, DMSO-d6) δ 8.26 (s, 1H), 8.18 (s, 1H), | 526.3 |
| | 7.96 (d, J = 9.6 Hz, 1H), 7.75 (s, 2H), 7.37 (s, 1H), 7.33 (t, J = | |
| | 8.0 Hz, 1H), 6.77 (d, J = 7.7 Hz, 1H), 4.92 (s, 2H), 3.76 (s, | |
| | 2H), 3.33-3.21 (m, 4H), 3.03 (dd, J = 28.2, 14.1 Hz, 2H), 2.73 | |
| | (s, 3H), 2.03-1.93 (m, 2H), 1.77-1.62 (m, 4H), 1.23 (s, 3H) | |
| 283 | ¹H NMR (400 MHz, DMSO-d6) δ 8.28 (br s, 2H), 8.17 (s, 1H), | 548.1 |
| | 8.09 (s, 1H), 8.04 (s, 1H), 7.92 (dd, J = 8.3, 1.8 Hz, 1H), 7.40 | |
| | (t, J = 1.8 Hz, 1H), 7.34 (t, J = 8.0 Hz, 1H), 6.79 (d, J = 7.6 | |
| | Hz, 1H), 5.09 (s, 2H), 3.93 (s, 2H), 3.35-3.20 (m, 4H), 3.09- | |
| | 2.95 (m, 2H), 2.73 (s, 3H), 1.14 (s, 9H) | |
| 284 | ¹H NMR (400 MHz, DMSO-d6) δ 8.40 (s, 1H), 8.19 (s, 1H), | 565.1 |
| | 8.05-7.95 (m, 3H), 6.67 (d, J = 1.1 Hz, 1H), 5.14 (s, 2H), | |
| | 3.82 (s, 2H), 3.22 (s, 2H), 3.02 (s, 3H), 2.38-2.30 (m, 3H), | |
| | 2.17-1.63 (m, 10H), 1.23 (s, 3H), 0.96-0.88 (m, 4H). | |
| 285 | ¹H NMR (400 MHz, DMSO-d6) δ 8.20 (s, 1H), 8.06 (s, 1H), | 569.6 |
| | 8.03 (s, 1H), 7.82 (s, 1H), 6.17 (s, 1H), 5.19 (s, 2H), 4.29 (q, | |
| | J = 7.0 Hz, 2H), 3.83 (s, 2H), 3.22 (s, 2H), 3.08 (s, 3H), 2.48- | |
| | 2.43 (m, 2H), 2.37-2.27 (m, 2H), 2.17-1.92 (m, 3H), 1.88-1.79 (m, | |
| | 1H), 1.79-1.61 (m, 4H), 1.33 (t, J = 7.0 Hz, 3H), 1.23 (s, 3H). | |
| 286 | ¹H NMR (400 MHz, DMSO-d6) δ 8.31 (s, 1H), 8.20 (s, 1H), | 585.1 |
| | 8.04 (d, J = 12.0 Hz, 2H), 7.94 (d, J = 1.0 Hz, 1H), 6.63 (d, J = | |
| | 1.0 Hz, 1H), 5.22 (s, 2H), 3.83 (s, 2H), 3.24 (s, 2H), 3.11 (q, | |
| | J = 14.0, 6.7 Hz, 2H), 3.08 (s, 3H), 2.49-2.44 (m, 2H), 2.33 | |
| | (dd, J = 20.3, 9.1 Hz, 2H), 2.11 (dd, J = 19.2, 8.2 Hz, 1H), | |
| | 2.04-1.94 (m, 2H), 1.88-1.78 (m, 1H), 1.78-1.61 (m, 4H), | |
| | 1.33 (t, J = 7.3 Hz, 3H), 1.23 (s, 3H). | |
| 287 | ¹H NMR (400 MHz, DMSO-d6) δ 8.48 (s, 1H), 8.06 (s, 1H), | 562.0 |
| | 8.02 (s, 1H), 7.94 (dd, J = 8.2, 1.8 Hz, 1H), 7.52 (s, 1H), 7.43 | |
| | (t, J = 8.0 Hz, 1H), 6.93 (d, J = 7.8 Hz, 1H), 5.32 (d, J = 6.9 | |
| | Hz, 2H), 5.08 (s, 2H), 5.04 (d, J = 7.0 Hz, 2H), 3.84 (s, 2H), | |
| | 3.11 (s, 3H), 2.05-1.93 (m, 2H), 1.77-1.62 (m, 4H), 1.24 (s, 3H). | |
| 288 | ¹H NMR (400 MHz, DMSO-d6) δ 8.22 (s, 1H), 8.17 (s, 1H), | 542.2 |
| | 7.96-7.83 (m, 3H), 7.41 (s, 1H), 7.34 (t, J = 8.0 Hz, 1H), 7.41- | |
| | 7.12 (m, 1H), 6.76 (d, J = 7.8 Hz, 1H), 5.05 (s, 2H), 3.80 (s, | |
| | 2H), 3.33-3.28 (m, 2H), 3.26-3.23 (m, 2H), 3.02 (q, J = | |
| | 13.8 Hz, 2H), 2.73 (s, 3H), 2.04-1.94 (m, 2H), 1.77-1.63 | |
| | (m, 4H), 1.24 (s, 3H). | |
| 289 | ¹H NMR (400 MHz, DMSO-d6) δ 8.36 (s, 1H), 8.29 (s, 1H), | 526.1 |
| | 7.95 (dd, J = 8.2, 1.5 Hz, 1H), 7.89 (d, J = 25.2 Hz, 2H), 7.56 | |
| | (s, 1H), 7.37 (t, J = 8.0 Hz, 1H), 7.27 (t, J = 55.0 Hz, 1H), 6.95 | |
| | (d, J = 7.8 Hz, 1H), 6.28 (d, J = 45.9 Hz, 1H), 5.37 (d, J = 6.6 | |
| | Hz, 1H), 5.21 (d, J = 5.9 Hz, 1H), 5.13 (dd, J = 6.9, 1.7 Hz, 1H), | |
| | 5.08-5.00 (m, 2H), 4.83 (dd, J = 6.1, 4.0 Hz, 1H), 3.79 (s, 2H), | |
| | 3.19 (s, 3H), 2.05-1.94 (m, 2H), 1.77-1.62 (m, 4H), 1.24 (s, 3H). | |
| 290 | ¹H NMR (400 MHz, DMSO-d6) δ 8.25 (s, 1H), 8.17 (s, 1H), | 570.6 |
| | 8.08-8.00 (m, 2H), 7.41 (s, 1H), 6.61 (t, J = 5.4 Hz, 1H), 5.90 | |
| | (s, 1H), 5.14 (s, 2H), 4.90 (d, J = 6.0 Hz, 2H), 4.78 (d, J = 6.0 | |
| | Hz, 2H), 3.86 (s, 2H), 3.49 (s, 2H), 3.24-3.14 (m, 5H), 2.08-1.98 | |
| | (m, 2H), 1.80-1.64 (m, 4H), 1.26 (s, 3H), 1.12 (t, J = 7.1 Hz, 3H) | |
| 291 | ¹H NMR (400 MHz, DMSO-d6) δ 8.25 (s, 1H), 7.97 (s, 1H), | 627.7 |
| | 7.91 (s, 1H), 7.40 (s, 1H), 6.61 (t, J = 5.4 Hz, 1H), 5.89 (s, | |
| | 1H), 5.14 (s, 2H), 4.90 (d, J = 6.1 Hz, 2H), 4.78 (d, J = 6.0 Hz, | |
| | 2H), 4.25-4.18 (m, 1H), 3.48 (s, 2H), 3.25-3.14 (m, 7H), | |
| | 2.69-2.61 (m, 2H), 2.31-2.15 (m, 6H), 2.07-1.95 (m, 2H), | |
| | 1.12 (t, J = 7.1 Hz, 3H), 0.98-0.84 (m, 6H). | |
| 292 | ¹H NMR (400 MHz, DMSO-d6) δ 8.22 (s, 1H), 8.17-8.00 (m, | 512.2 |
| | 3H), 7.80 (t, J = 7.6 Hz, 1H), 6.79 (s, 1H), 5.22 (s, 2H), 4.96 | |
| | (d, J = 6.2 Hz, 2H), 4.84 (d, J = 6.2 Hz, 2H), 3.17 (s, 3H), 2.67 | |
| | (d, J = 7.3 Hz, 2H), 2.25-2.13 (m, 1H), 1.72-1.44 (m, 6H), | |
| | 1.27-1.11 (m, 2H). | |
| 293 | ¹H NMR (400 MHz, DMSO-d6) δ 8.23 (s, 1H), 7.98 (s, 1H), | 668.4 |
| | 7.91 (s, 1H), 7.87 (d, J = 0.9 Hz, 1H), 6.38 (d, J = 0.9 Hz, 1H), | |
| | 5.29-5.22 (m, 1H), 5.17 (s, 2H), 4.87 (d, J = 6.2 Hz, 2H), | |
| | 4.79 (d, J = 6.3 Hz, 2H), 4.19 (d, J = 14.5 Hz, 1H), 3.53 (s, | |
| | 2H), 3.39-3.32 (m, 1H), 3.28 (s, 3H), 2.64-2.55 (m, 2H), | |
| | 2.28-2.16 (m, 3H), 2.12 (s, 3H), 2.01-1.87 (m, 4H), 1.77- | |
| | 1.51 (m, 7H), 0.91 (d, J = 6.6 Hz, 3H), 0.86 (d, J = 6.6 Hz, 3H) | |
| 294 | ¹H NMR (400 MHz, DMSO-d6) δ 8.23 (s, 1H), 8.04 (s, 1H), | 611.5 |
| | 8.00 (s, 1H), 7.88 (s, 1H), 6.38 (s, 1H), 5.29-5.22 (m, 1H), | |
| | 5.17 (s, 2H), 4.88 (d, J = 6.2 Hz, 2H), 4.80 (d, J = 6.2 Hz, 2H), | |
| | 3.81 (s, 2H), 3.53 (s, 2H), 3.29 (s, 3H), 2.02-1.91 (m, 4H), | |
| | 1.77-1.55 (m, 10H), 1.21 (s, 3H). | |
| 295 | ¹H NMR (400 MHz, DMSO-d6) δ 8.20 (s, 1H), 8.02 (s, 1H), | 608.3 |
| | 8.00 (s, 1H), 7.39 (s, 1H), 6.52 (d, J = 6.1 Hz, 1H), 5.77 (s, | |
| | 1H), 5.14 (s, 2H), 3.95 (h, J = 6.0 Hz, 1H), 3.83 (s, 2H), 3.15 | |
| | (s, 2H), 3.03 (s, 3H), 2.48-2.39 (m, 2H), 2.26 (dd, J = 18.9, | |
| | 9.3 Hz, 2H), 2.14-1.95 (m, 3H), 1.95-1.85 (m, 2H), 1.85- | |
| | 1.78 (m, 1H), 1.78-1.63 (m, 6H), 1.60-1.50 (m, 2H), 1.50- | |
| | 1.39 (m, 2H), 1.24 (s, 3H) | |
| 296 | ¹H NMR (400 MHz, DMSO-d6) δ 8.20 (s, 1H), 8.05 (d, J = 7.5 | 511.1 |
| | Hz, 1H), 8.03 (d, J = 8.2 Hz, 1H), 7.78 (t, J = 7.9 Hz, 1H), 7.40 | |
| | (s, 1H), 6.53 (d, J = 5.9 Hz, 1H), 5.77 (s, 1H), 5.18 (s, 2H), | |
| | 3.95 (h, J = 6.3 Hz, 1H), 3.15 (s, 2H), 3.02 (s, 3H), 2.47-2.38 | |
| | (m, 2H), 2.26 (dd, J = 18.5, 9.3 Hz, 2H), 2.15-2.03 (m, 1H), | |
| | 1.96-1.85 (m, 2H), 1.85-1.74 (m, 1H), 1.73-1.60 (m, 2H), | |
| | 1.60-1.50 (m, 2H), 1.50-1.38 (m, 2H) | |
| 297 | ¹H NMR (400 MHz, DMSO-d6) δ 8.25 (s, 1H), 7.97 (s, 1H), | 667.5 |
| | 7.91 (s, 1H), 7.39 (s, 1H), 6.63 (d, J = 6.1 Hz, 1H), 5.93 (s, | |
| | 1H), 5.14 (s, 2H), 4.93-4.74 (m, 4H), 4.21 (d, J = 14.5 Hz, | |
| | 1H), 4.01-3.91 (m, 1H), 3.48 (s, 3H), 3.23 (s, 5H), 2.69- | |
| | 2.56 (m, 2H), 2.32-2.11 (m, 7H), 1.98-1.86 (m, 4H), 1.70- | |
| | 1.41 (m, 6H), 0.96-0.84 (m, J = 19.7, 6.6 Hz, 6H) | |
| 298 | ¹H NMR (400 MHz, DMSO-d6) δ 8.25 (s, 1H), 8.07-7.98 (m, | 610.4 |
| | 2H), 7.40 (s, 1H), 6.62 (d, J = 6.1 Hz, 1H), 5.94 (s, 1H), 5.14 | |
| | (s, 2H), 4.92-4.73 (m, 4H), 4.01-3.91 (m, 1H), 3.83 (s, 2H), | |
| | 3.49 (s, 2H), 3.24 (s, 3H), 2.05-1.86 (m, 4H), 1.80-1.39 (m, | |
| | 10H), 1.24 (s, 3H) | |
| 299 | ¹H NMR (400 MHz, DMSO-d6) δ 8.57 (d, J = 1.3 Hz, 1H), 8.22 | 607.4 |
| | (s, 1H), 8.04 (s, 1H), 7.98 (s, 1H), 7.65 (d, J = 1.3 Hz, 1H), | |
| | 5.21 (s, 2H), 4.24-4.20 (m, 1H), 3.42-3.38 (m, 1H), 3.23 (s, | |
| | 3H), 2.67-2.59 (m, 2H), 2.44-2.07 (m, 10H), 2.03-1.78 | |
| | (m, 2H), 1.00-0.81 (m, 6H). | |
| 300 | ¹H NMR (400 MHz, CD₃CN) δ 8.09-8.06 (m, 1H), 8.03 (s, | 567.2 |
| | 1H), 7.98-7.92 (m, 2H), 6.81 (d, J = 1.3 Hz, 1H), 5.13 (s, | |
| | 2H), 5.00-4.92 (m, 4H), 3.87 (s, 2H), 3.55 (s, 2H), 3.18 (s, | |
| | 3H), 2.06-1.99 (m, 2H), 1.85-1.73 (m, 4H), 1.30 (s, 3H), | |
| | 1.01-0.92 (m, 4H) | |
| 301 | ¹H NMR (400 MHz, CD₃CN) δ 8.04-7.85 (m, 4H), 7.37-7.26 | 526.2 |
| | (m, 2H), 6.75 (d, J = 7.6 Hz, 1H), 5.05-4.90 (m, 6H), 3.86 (s, | |
| | 2H), 3.52 (s, 2H), 2.85 (s, 3H), 1.86-1.64 (m, 6H), 1.29 (s, 3H). | |
| 302 | ¹H NMR (400 MHz, DMSO-d6) δ 8.18 (s, 2H), 8.01 (d, J = 8.9 | 681.4 |
| | Hz, 2H), 7.35 (s, 1H), 6.46 (d, J = 7.0 Hz, 1H), 5.78 (s, 1H), | |
| | 5.10 (s, 2H), 3.84 (s, 2H), 3.42 (t, J = 5.8 Hz, 4H), 3.22 (s, | |
| | 3H), 3.13 (s, 2H), 3.02 (s, 3H), 2.86 (d, J = 11.5 Hz, 2H), 2.41 | |
| | (m, 2H), 2.24 (m, 2H), 2.13-1.97 (m, 5H), 1.88 (m, 2H), 1.83- | |
| | 1.61 (m, 5H), 1.44-1.31 (m, 2H), 1.24 (s, 3H). | |
| 303 | ¹H NMR (400 MHz, DMSO-d6) δ 8.19 (s, 1H), 7.99 (d, J = 8.5 | 667.4 |
| | Hz, 2H), 7.36 (d, J = 0.7 Hz, 1H), 6.58 (t, J = 5.5 Hz, 1H), 5.72 | |
| | (s, 1H), 5.11 (s, 2H), 3.81 (s, 2H), 3.58-3.51 (m, 4H), 3.21- | |
| | 3.11 (m, 4H), 3.00 (s, 3H), 2.42 (m, 2H), 2.36-2.18 (m, 8H), | |
| | 2.13-1.91 (m, 3H), 1.86-1.58 (m, 7H), 1.22 (s, 3H). | |
| 304 | ¹H NMR (400 MHz, DMSO-d6) δ 8.26 (s, 1H), 8.10-8.01 (m, | 571.3 |
| | 2H), 7.93 (s, 1H), 6.42 (s, 1H), 5.20 (s, 2H), 4.95-4.80 (m, | |
| | 4H), 4.32 (q, J = 7.0 Hz, 2H), 3.82 (s, 2H), 3.56 (s, 2H), 3.31 | |
| | (s, 3H), 2.03-1.92 (m, 2H), 1.77-1.62 (m, 4H), 1.33 (t, J = | |
| | 7.0 Hz, 2H), 1.23 (s, 3H). | |
| 305 | ¹H NMR (400 MHz, CD₃OD) δ 8.48 (s, 1H), 8.09 (s, 1H), 7.96 | 508.1 |
| | (s, 1H), 7.82 (dd, J = 8.2, 1.5 Hz, 1H), 7.38 (t, J = 8.0 Hz, 1H), | |
| | 7.30-6.93 (m, 4H), 6.75-6.65 (m, 1H), 5.20 (d, J = 6.4 Hz, 2H), | |
| | 5.08 (d, J = 6.4 Hz, 2H), 5.05 (s, 2H), 4.97 (s, 2H), 4.24 (s, 2H), | |
| | 2.50-2.37 (m, 2H), 2.17-1.92 (m, 4H), 1.64 (s, 3H), 1.56 (s, 3H). | |
| 306 | ¹H NMR (400 MHz, DMSO-d6) δ 8.35 (s, 2H), 8.20 (s, 1H), | 637.5 |
| | 8.03-7.99 (m, 2H), 7.41 (s, 1H), 6.51 (d, J = 5.3 Hz, 1H), 5.94 | |
| | (s, 1H), 5.13 (s, 2H), 3.84-3.74 (m, 3H), 3.16 (s, 2H), 3.07- | |
| | 3.05 (m, 4H), 2.54 (s, 3H), 2.43 (d, J = 8.1 Hz, 1H), 2.33- | |
| | 2.22 (m, 2H), 2.12-1.93 (m, 3H), 1.89-1.79 (m, 2H), 1.76- | |
| | 1.61 (m, 9H), 1.23 (s, 3H). | |
| 307 | ¹H NMR (400 MHz, DMSO-d6) δ 8.40 (s, 1H), 8.01 (d, J = 8.3 | 625.2 |
| | Hz, 2H), 7.80 (t, J = 5.6 Hz, 1H), 7.41 (s, 1H), 6.83 (t, J = 5.7 | |
| | Hz, 1H), 5.92 (s, 1H), 5.07 (s, 2H), 3.81 (s, 2H), 3.74 (d, J = | |
| | 5.8 Hz, 2H), 3.16 (s, 2H), 3.13-3.01 (m, 5H), 2.46-2.39 (m, | |
| | 1H), 2.26-2.23 (m, 2H), 2.14-1.94 (m, 4H), 1.82 (s, 1H), 1.76- | |
| | 1.63 (m, 4H), 1.23 (s, 3H), 0.99 (t, J = 7.2 Hz, 3H). | |
| 308 | ¹H NMR (400 MHz, DMSO-d6) δ 8.18 (s, 1H), 8.15 (s, 1H), | 593.2 |
| | 8.06 (s, 1H), 8.03 (s, 1H), 7.46 (s, 1H), 6.98 (t, J = 5.7 Hz, | |
| | 1H), 5.87 (s, 1H), 5.20 (s, 2H), 3.88 (s, 2H), 3.49 (q, J = 5.8 | |
| | Hz, 2H), 3.17 (s, 2H), 3.04 (s, 3H), 2.80 (t, J = 6.4 Hz, 2H), | |
| | 2.47-2.41 (m, 2H), 2.31-2.22 (m, 2H), 2.13-1.98 (m, 3H), | |
| | 1.87-1.64 (m, 5H), 1.27 (s, 3H). | |
| 309 | ¹H NMR (400 MHz, DMSO-d6) δ 8.22 (s, 1H), 8.19 (s, 1H), | 665.3 |
| | 8.04-7.98 (m, J = 9.2 Hz, 2H), 7.40 (d, J = 1.0 Hz, 1H), 6.50 | |
| | (t, J = 5.4 Hz, 1H), 5.78 (s, 1H), 5.15 (s, 2H), 3.82 (s, 2H), | |
| | 3.15 (s, 2H), 3.03 (s, 3H), 2.88 (d, J = 11.3 Hz, 2H), 2.46- | |
| | 2.40 (m, 4H), 2.30-2.19 (m, 2H), 2.12-1.90 (m, 7H), 1.89- | |
| | 1.62 (m, 6H), 1.57 (d, J = 13.9 Hz, 2H), 1.36-1.28 (m, 1H), | |
| | 1.23 (s, 3H), 1.21-1.09 (m, 2H), 0.89 (d, J = 6.5 Hz, 3H). | |
| 310 | ¹H NMR (400 MHz, DMSO-d6) δ 8.28 (s, 1H), 8.03-7.97 (m, | 554.1 |
| | 2H), 7.41 (s, 1H), 6.49 (d, J = 4.8 Hz, 1H), 5.69 (s, 1H), 5.15 | |
| | (s, 2H), 3.82 (s, 2H), 3.16 (s, 2H), 3.01 (s, 3H), 2.71 (d, J = | |
| | 4.7 Hz, 3H), 2.44-2.42 (m, 2H), 2.36-2.20 (m, 3H), 2.18- | |
| | 1.92 (m, 3H), 1.88-1.61 (m, 5H), 1.23 (s, 3H) | |
| 311 | ¹H NMR (400 MHz, DMSO-d6) δ 8.07-8.02 (m, 3H), 7.41 (s, | 625.2 |
| | 1H), 6.79-6.52 (m, 1H), 5.96 (s, 1H), 5.08 (s, 2H), 4.04 (d, J = | |
| | 5.7 Hz, 2H), 3.86 (s, 2H), 3.16 (s, 2H), 3.09-3.02 (m, 6H), | |
| | 2.84 (s, 3H), 2.47-2.40 (m, 1H), 2.31-2.21 (m, 2H), 2.16- | |
| | 1.97 (m, 3H), 1.87-1.62 (m, 6H), 1.26 (s, 3H). | |
| 312 | ¹H NMR (400 MHz, DMSO-d6) δ 8.19 (s, 1H), 8.01 (d, J = | 661.2 |
| | 10.8 Hz, 2H), 7.47 (s, 1H), 6.92 (q, J = 4.7 Hz, 1H), 6.72 (t, J = | |
| | 5.8 Hz, 1H), 5.81 (s, 1H), 5.18 (s, 2H), 3.82 (s, 2H), 3.59 | |
| | (dd, J = 13.1, 6.5 Hz, 2H), 3.27-3.22 (m, 2H), 3.17 (s, 2H), | |
| | 3.04 (s, 3H), 2.58 (d, J = 4.8 Hz, 3H), 2.47-2.40 (m, 2H), | |
| | 2.26 (dd, J = 19.2, 9.4 Hz, 2H), 2.09 (dd, J = 18.5, 8.9 Hz, | |
| | 1H), 2.04-1.92 (m, 2H), 1.87-1.77 (m, 1H), 1.77-1.62 (m, | |
| | 4H), 1.23 (s, 3H) | |
| 313 | ¹H NMR (400 MHz, DMSO-d6) δ 8.16 (s, 1H), 8.15 (s, 1H), | 634.2 |
| | 8.01 (s, 1H), 7.99 (s, 1H), 7.52 (s, 1H), 7.37 (s, 1H), 7.30 (s, 1H), | |
| | 6.80 (t, J = 5.5 Hz, 1H), 5.81 (s, 1H), 5.13 (s, 2H), 4.20 (d, | |
| | J = 5.4 Hz, 2H), 3.82 (s, 2H), 3.74 (s, 3H), 3.12 (s, 2H), 2.98 | |
| | (s, 3H), 2.41 (t, J = 10.0 Hz, 2H), 2.28-2.14 (m, 2H), 2.08-1.85 | |
| | (m, 3H), 1.82-1.56 (m, 6H), 1.23 (s, 3H). Missing one proton | |
| 314 | 1H NMR (400 MHz, DMSO-d6) δ 8.23 (s, 1H), 8.20 (s, 1H), | 653.3 |
| | 8.01 (d, J = 10.0 Hz, 2H), 7.81 (t, J = 5.3 Hz, 1H), 7.39 (s, | |
| | 1H), 6.56 (t, J = 5.5 Hz, 1H), 5.72 (s, 1H), 5.14 (s, 2H), 3.82 | |
| | (s, 2H), 3.19-3.14 (m, 5H), 3.04 (dd, J = 11.5, 5.5 Hz, 2H), | |
| | 3.01 (s, 3H), 2.48-2.40 (m, 2H), 2.32-2.21 (m, 2H), 2.15- | |
| | 2.04 (m, 1H), 2.03-1.94 (m, 2H), 1.86-1.79 (m, 1H), 1.77 | |
| | (s, 3H), 1.76-1.63 (m, 4H), 1.54-1.38 (m, 4H), 1.23 (s, 3H) | |
| 315 | ¹H NMR (400 MHz, DMSO-d6) δ 8.20 (s, 1H), 8.01 (d, J = 9.0 | 686.3 |
| | Hz, 2H), 7.41 (s, 1H), 6.82 (t, J = 5.6 Hz, 1H), 5.75 (s, 1H), | |
| | 5.18 (s, 2H), 3.82 (s, 2H), 3.15 (s, 2H), 3.14-3.08 (m, 4H), | |
| | 3.05 (s, 1H), 3.02 (s, 3H), 2.48-2.40 (m, 2H), 2.31-2.21 (m, | |
| | 2H), 2.13-1.95 (m, 6H), 1.87-1.78 (m, 1H), 1.75-1.56 (m, | |
| | 6H), 1.23 (s, 4H) | |
| 316 | 1H NMR (400 MHz, DMSO-d6) δ 10.82 (s, 1H), 8.20 (br s, | 683.2 |
| | 1H), 8.15 (s, 1H), 8.01 (s, 1H), 7.98 (s, 1H), 7.52 (d, J = 7.8 | |
| | Hz, 1H), 7.38 (s, 1H), 7.32 (d, J = 8.1 Hz, 1H), 7.13 (d, J = 1.8 | |
| | Hz, 1H), 7.03 (t, J = 7.3 Hz, 1H), 6.94 (t, J = 7.4 Hz, 1H), 6.66 | |
| | (t, J = 5.6 Hz, 1H), 5.74 (s, 1H), 5.18 (s, 2H), 3.81 (s, 2H), | |
| | 3.46 (q, J = 6.7 Hz, 2H), 3.13 (s, 2H), 2.99 (s, 3H), 2.91 (t, J = | |
| | 7.5 Hz, 2H), 2.45-2.38 (m, 2H), 2.28-2.19 (m, 2H), 2.13- | |
| | 1.90 (m, 3H), 1.85-1.59 (m, 5H), 1.22 (s, 3H). | |
| 317 | ¹H NMR (400 MHz, DMSO-d6) δ 8.24 (s, 1H), 8.19 (br s, 1H), | 634.1 |
| | 8.10 (s, 1H), 8.08 (s, 1H), 7.48 (s, 1H), 6.93 (t, J = 6.3 Hz, | |
| | 1H), 6.03 (s, 1H), 5.51 (t, J = 6.2 Hz, 1H), 5.21 (s, 2H), 3.93 | |
| | (s, 2H), 3.86 (td, J = 14.4, 6.1 Hz, 2H), 3.65 (td, J = 13.9, 6.1 | |
| | Hz, 2H), 3.21 (s, 2H), 3.08 (s, 3H), 2.51-2.45 (m, 2H), 2.36- | |
| | 2.27 (m, 2H), 2.18-2.03 (m, 3H), 1.93-1.68 (m, 5H), 1.32 (s, 3H). | |
| 318 | ¹H NMR (400 MHz, DMSO-d6) δ 8.17 (s, 1H), 8.00 (d, J = 3.3 | 687.2 |
| | Hz, 2H), 7.36 (s, 1H), 7.29-7.14 (m, 4H), 7.09 (d, J = 7.5 Hz, | |
| | 1H), 5.81 (s, 1H), 5.04 (s, 2H), 4.39 (d, J = 5.9 Hz, 2H), 3.81 | |
| | (s, 2H), 3.13 (s, 2H), 2.92 (s, 3H), 2.47-2.35 (m, 3H), 2.28- | |
| | 2.15 (m, 3H), 2.11-2.03 (m, 8H), 2.03-1.91 (m, 2H), 1.82- | |
| | 1.60 (m, 5H), 1.23 (s, 3H). | |
| 319 | ¹H NMR (400 MHz, DMSO-d6) δ 8.38 (s, 1H), 8.17 (s, 1H), | 560.1 |
| | 8.05 (s, 1H), 8.02 (s, 1H), 7.91 (dd, J = 8.1, 1.5 Hz, 1H), 7.40 | |
| | (s, 1H), 7.34 (t, J = 8.0 Hz, 1H), 6.78 (d, J = 8.1 Hz, 1H), 5.09 | |
| | (s, 2H), 4.11-3.81 (m, 2H), 3.37-3.12 (m, 5H), 3.11-2.95 | |
| | (m, 2H), 2.72 (s, 3H), 1.91-1.79 (m, 1H), 1.09 (d, J = 6.3 Hz, | |
| | 3H), 0.73-0.59 (m, 1H), 0.47-0.38 (m, 1H), 0.33 (ddd, J = | |
| | 13.2, 9.1, 4.1 Hz, 1H), 0.14 (td, J = 9.2, 5.0 Hz, 1H), −0.00 (td, | |
| | J = 9.3, 5.1 Hz, 1H). | |
| 320 | ¹H NMR (400 MHz, DMSO-d6) δ 8.18 (s, 1H), 8.01 (d, J = 8.4 | 634.2 |
| | Hz, 2H), 7.47 (s, 1H), 7.38 (d, J = 0.9 Hz, 1H), 6.90 (s, 1H), | |
| | 6.76 (t, J = 5.6 Hz, 1H), 5.90 (d, J = 0.8 Hz, 1H), 5.15 (s, 2H), | |
| | 4.24 (d, J = 5.5 Hz, 2H), 3.82 (s, 2H), 3.58 (s, 3H), 3.15 (s, | |
| | 2H), 3.02 (s, 3H), 2.47-2.39 (m, 2H), 2.29-2.18 (m, 2H), 2.13- | |
| | 1.94 (m, 3H), 1.86-1.77 (m, 1H), 1.76-1.62 (m, 4H), 1.23 (s, 3H). | |
| 321 | ¹H NMR (400 MHz, DMSO-d6) δ 8.47 (d, J = 5.8 Hz, 2H), 8.19 | 645.2 |
| | (s, 1H), 8.04 (d, J = 7.6 Hz, 2H), 7.43 (s, 1H), 7.28 (d, J = 5.9 | |
| | Hz, 2H), 6.78 (t, J = 5.7 Hz, 1H), 5.78 (s, 1H), 5.22 (s, 2H), | |
| | 3.87 (s, 2H), 3.46 (dd, J = 14.0, 6.4 Hz, 2H), 3.16 (s, 2H), 3.03 | |
| | (s, 3H), 2.86 (t, J = 7.3 Hz, 2H), 2.44 (d, J = 8.1 Hz, 2H), 2.26 | |
| | (dd, J = 20.1, 9.0 Hz, 2H), 2.09 (dd, J = 23.6, 13.1 Hz, 3H), | |
| | 1.75 (t, J = 25.1 Hz, 5H), 1.27 (s, 3H). | |
| 322 | ¹H NMR (400 MHz, DMSO-d6) δ 8.17 (s, 1H), 8.06-7.99 (m, | 609.2 |
| | 3H), 6.54 (s, 1H), 5.18 (s, 2H), 3.82 (s, 2H), 3.22 (s, 2H), 2.92 | |
| | (s, 3H), 2.64-2.58 (m, 1H), 2.38-2.28 (m, 3H), 2.19-2.08 | |
| | (m, 1H), 2.04-1.93 (m, 2H), 1.88-1.79 (m, 1H), 1.77-1.63 | |
| | (m, 4H), 1.51-1.43 (m, 2H), 1.23 (s, 3H), 0.94 (s, 9H) | |
| 323 | ¹H NMR (400 MHz, DMSO-d6) δ 8.20 (s, 1H), 8.04 (d, J = | 571.1 |
| | 11.2 Hz, 2H), 7.95 (d, J = 1.1 Hz, 1H), 6.63 (d, J = 1.1 Hz, 1H), | |
| | 5.22 (s, 2H), 3.82 (d, J = 7.4 Hz, 2H), 3.24 (s, 2H), 3.07 (s, 3H), | |
| | 2.50 (s, 3H), 2.39-2.28 (m, 2H), 2.18-2.07 (m, 1H), 2.04-1.93 | |
| | (m, 2H), 1.90-1.78 (m, 1H), 1.76-1.62 (m, 4H), 1.23 (s, 3H). | |
| 324 | ¹H NMR (400 MHz, DMSO-d6) δ 8.22 (s, 1H), 8.18 (s, 1H), | 601.2 |
| | 8.09-7.98 (m, 4H), 7.53 (t, J = 7.3 Hz, 2H), 7.46 (t, J = 7.2 | |
| | Hz, 1H), 7.28 (s, 1H), 5.35 (s, 2H), 3.84 (s, 2H), 3.03 (s, 3H), | |
| | 2.45-2.38 (m, 3H), 2.22-2.13 (m, 1H), 2.04-1.95 (m, 2H), | |
| | 1.92-1.84 (, 1H), 1.77-1.64 (m, 5H), 1.24 (s, 3H). | |
| 325 | ¹H NMR (400 MHz, DMSO-d6) δ 8.42 (s, 1H), 8.06-8.00 (m, | 629.2 |
| | 3H), 7.28-7.14 (m, 6H), 6.57 (s, 1H), 5.16 (s, 2H), 3.82 (s, | |
| | 2H), 3.19 (s, 2H), 2.97 (s, 4H), 2.85 (s, 3H), 2.29-2.25 (m, | |
| | 4H), 2.18-2.07 (m, 2H), 2.02-1.94 (m, 2H), 1.86-1.64 (m, | |
| | 6H), 1.23 (s, 3H). | |
| 326 | ¹H NMR (400 MHz, DMSO-d6) δ 8.30 (d, J = 5.3 Hz, 1H), 8.25 | 525.1 |
| | (s, 1H), 8.18 (s, 1H), 8.08-7.99 (m, 2H), 6.82-6.79 (m, 1H), | |
| | 5.20 (s, 2H), 3.83 (s, 2H), 3.25 (s, 2H), 3.00 (s, 3H), 2.41- | |
| | 2.29 (m, 3H), 2.21-1.95 (m, 3H), 1.89-1.79 (m, 1H), 1.79- | |
| | 1.64 (m, 5H), 1.23 (s, 3H). | |
| 327 | ¹H NMR (400 MHz, DMSO-d6) δ 8.35 (br s, 1H), 8.22 (s, 1H), | 605.2 |
| | 8.15 (s, 1H), 8.04 (s, 1H), 8.01 (s, 1H), 8.00 (d, J = 1.0 Hz, | |
| | 1H), 7.92 (s, 1H), 7.01 (d, J = 1.1 Hz, 1H), 5.25 (s, 2H), 3.88 | |
| | (s, 3H), 3.81 (s, 2H), 3.26-3.21 (m, 2H), 3.01 (s, 3H), 2.42- | |
| | 2.32 (m, 2H), 2.19-2.05 (m, 1H), 2.02-1.90 (m, 2H), 1.89- | |
| | 1.78 (m, 1H), 1.75-1.60 (m, 4H), 1.22 (s, 3H). | |
| 328 | 1H NMR (400 MHz, DMSO-d6) δ 8.37 (s, 1H), 8.30 (br s, 1H), | 560.1 |
| | 8.03 (s, 1H), 7.99 (s, 1H), 7.81 (d, J = 8.0 Hz, 1H), 7.46 (s, | |
| | 1H), 7.41-7.30 (m, 1H), 6.83 (d, J = 7.2 Hz, 1H), 5.04 (s, | |
| | 2H), 3.81 (s, 2H), 3.06-2.93 (m, 2H), 2.81 (s, 3H), 2.13- | |
| | 2.01 (m, 1H), 2.01-1.91 (m, 2H), 1.91-1.80 (m, 1H), 1.77- | |
| | 1.57 (m, 4H), 1.21 (s, 3H). | |
| 329 | ¹H NMR (400 MHz, DMSO-d6) δ 8.18 (s, 1H), 7.99 (d, J = 9.4 | 679.3 |
| | Hz, 2H), 7.37 (s, 1H), 6.71 (s, 1H), 5.75 (s, 1H), 5.13 (s, 2H), | |
| | 4.35 (d, J = 12.7 Hz, 1H), 3.80 (s, 3H), 3.17-2.84 (m, 10H), 2.33- | |
| | 2.15 (m, 2H), 2.14-1.55 (m, 15H), 1.21 (s, 3H), 1.14-0.86 (m, 2H). | |
| 330 | ¹H NMR (500 MHz, DMSO-d6) δ 8.38 (s, 2H), 7.89 (s, 1H), | 639.2 |
| | 7.84 (s, 1H), 7.15 (d, J = 1.2 Hz, 1H), 5.93 (s, 2H), 5.74 (d, J = | |
| | 1.2 Hz, 1H), 5.04 (s, 2H), 4.14 (d, J = 14.4 Hz, 1H), 3.09 (s, | |
| | 3H), 2.60-2.52 (m, 4H), 2.51 (s, 3H), 2.31-2.05 (m, 9H), | |
| | 1.87 (m, 2H), 1.77 (s, 1H), 1.17 (s, 2H), 0.87 (d, J = 6.7 Hz, | |
| | 3H), 0.82 (d, J = 6.7 Hz, 3H). | |
| 331 | ¹H NMR (400 MHz, DMSO-d6) δ 8.27 (s, 1H), 8.05 (s, 1H), | 526.1 |
| | 8.01 (s, 1H), 7.94 (dd, J = 8.2, 1.9 Hz, 1H), 7.34-7.26 (m, | |
| | 2H), 7.22 (s, 1H), 6.60 (d, J = 7.7 Hz, 1H), 5.10-5.00 (m, | |
| | 4H), 4.95-4.83 (m, 4H), 3.83 (s, 2H), 2.06-1.91 (m, 2H), | |
| | 1.83-1.59 (m, 4H), 1.49 (s, 3H), 1.23 (s, 3H). | |
| 332 | ¹H NMR (400 MHz, DMSO-d6) δ 8.17 (s, 1H), 8.07 (s, 1H), | 562.1 |
| | 8.03 (s, 1H), 7.97-7.83 (m, 1H), 7.39 (ap s, 1H), 7.34 (t, J = | |
| | 8.0 Hz, 1H), 6.79 (d, J = 7.9 Hz, 1H), 5.09 (s, 2H), 4.49 (d, J = | |
| | 5.8 Hz, 2H), 4.20 (d, J = 5.9 Hz, 2H), 3.91 (s, 2H), 3.30- | |
| | 3.13 (m, 5H), 3.10-2.94 (m, 2H), 2.73 (s, 3H), 1.43 (s, 3H). | |
| 333 | ¹H NMR (400 MHz, DMSO-d6) δ 10.62 (s, 1H), 8.35 (s, 1H), | 673.2 |
| | 8.16 (s, 1H), 7.98 (s, 1H), 7.91 (s, 2H), 7.54 (s, 1H), 5.17 (s, | |
| | 2H), 4.36 (s, 2H), 4.19 (d, J = 14.3 Hz, 1H), 3.22 (m, 4H), 3.05 | |
| | (s, 3H), 2.67-2.54 (m, 2H), 2.41-2.16 (m, 6H), 2.16-2.07 | |
| | (m, 4H), 1.98-1.74 (m, 3H), 0.91 (d, J = 6.7 Hz, 3H), 0.86 | |
| | (d, J = 6.6 Hz, 3H) | |
| 334 | ¹H NMR (400 MHz, DMSO-d6) δ 8.17 (s, 1H), 8.03 (s, 1H), | 546.1 |
| | 8.00 (s, 1H), 7.91 (dd, J = 8.2, 1.4 Hz, 1H), 7.40 (t, J = 1.8 Hz, | |
| | 1H), 7.34 (t, J = 8.0 Hz, 1H), 6.79 (d, J = 7.8 Hz, 1H), 5.09 (s, | |
| | 2H), 3.91 (s, 2H), 3.30-3.22 (m, 4H), 3.08-2.96 (m, 2H), | |
| | 2.72 (s, 3H), 2.38 (d, J = 6.6 Hz, 2H), 0.95-0.84 (m, 1H), | |
| | 0.42-0.37 (m, 2H), 0.10-0.06 (m, 2H). | |
| 335 | ¹H NMR (400 MHz, DMSO-d6) δ 8.19 (s, 1H), 8.04 (s, 1H), | 638.3 |
| | 8.01 (s, 1H), 7.38 (s, 1H), 6.70 (t, J = 5.2 Hz, 1H), 5.77 (s, | |
| | 1H), 5.14 (s, 1H), 3.85 (ap s, 4H), 3.30-3.13 (m, 3H), 3.08 | |
| | (t, J = 5.8 Hz, 2H), 3.02 (s, 3H), 2.54 (s, 3H), 2.39-2.15 (m, | |
| | 2H), 2.15-1.94 (m, 3H), 1.79-1.63 (m, 5H), 1.63-1.49 (m, | |
| | 3H), 1.36-1.10 (m, 6H), 0.86 (t, J = 7.3 Hz, 2H). | |
| 336 | ¹H NMR (400 MHz, DMSO-d6) δ 8.24 (s, 1H), 8.17 (s, 1H), | 642.2 |
| | 8.00 (d, J = 9.3 Hz, 2H), 7.39 (s, 1H), 6.55 (t, J = 5.3 Hz, 1H), | |
| | 5.81 (s, 1H), 5.14 (s, 2H), 3.82 (s, 2H), 3.52 (m, 4H), 3.46- | |
| | 3.40 (m, 2H), 3.34 (m, 2H), 3.22 (s, 3H), 3.17-3.10 (m, 2H), | |
| | 3.05-2.97 (s, 3H), 2.41 (m, 2H), 2.31-2.18 (m, 2H), 2.12- | |
| | 1.91 (m, 3H), 1.86-1.60 (m, 5H), 1.23 (s, 3H) | |
| 337 | ¹H NMR (400 MHz, DMSO-d6) δ 8.17 (s, 1H), 7.99 (d, J = | 611.2 |
| | 10.6 Hz, 2H), 7.39 (s, 1H), 6.46 (t, J = 5.3 Hz, 1H), 5.77 (s, | |
| | 1H), 5.13 (s, 2H), 3.79 (m, 2H), 3.28 (m, 2H), 3.13 (s, 2H), | |
| | 3.02 (s, 3H), 2.39 (m, 4H), 2.30-2.14 (m, 9H), 2.12-1.92 | |
| | (m, 3H), 1.73 (m, 5H), 1.22 (s, 3H). | |
| 338 | ¹H NMR (400 MHz, DMSO-d6) δ 8.16 (s, 1H), 7.99 (d, J = 9.9 | 651.2 |
| | Hz, 2H), 7.41 (s, 1H), 6.66 (d, J = 5.3 Hz, 1H), 5.74 (s, 1H), | |
| | 5.14 (d, J = 20.2 Hz, 2H), 3.81 (s, 2H), 3.39 (t, J = 6.9 Hz, | |
| | 2H), 3.30 (s, 4H), 3.14 (s, 2H), 3.00 (s, 3H), 2.43 (t, J = 8.7 | |
| | Hz, 2H), 2.30-2.13 (m, 4H), 2.13-1.59 (m, 10H), 1.22 (s, 3H). | |
| 339 | ¹H NMR (400 MHz, DMSO-d6) δ 8.36 (s, 2H), 8.19 (s, 1H), | 637.2 |
| | 8.00 (d, J = 9.4 Hz, 2H), 7.39 (s, 1H), 6.75 (s, 1H), 5.74 (s, | |
| | 1H), 5.14 (s, 2H), 3.81 (s, 2H), 3.28-3.10 (m, 4H), 3.04 (m, | |
| | 5H), 2.73 (s, 2H), 2.43 (s, 2H), 2.24 (dd, J = 19.0, 9.0 Hz, 2H), | |
| | 2.14-1.58 (m, 12H), 1.39-1.15 (m, 5H). | |
| 340 | ¹H NMR (400 MHz, DMSO-d6) δ 8.18 (s, 1H), 8.00 (d, J = 8.8 | 653.2 |
| | Hz, 2H), 7.39 (s, 1H), 6.51 (t, J = 5.3 Hz, 1H), 5.77 (s, 1H), | |
| | 5.13 (s, 2H), 3.82 (s, 2H), 3.61-3.51 (m, 4H), 3.37-3.25 (m, | |
| | 2H), 3.14 (s, 2H), 3.02 (s, 3H), 2.48-2.37 (m, 8H), 2.24 (m, | |
| | 2H), 2.13-1.91 (m, 3H), 1.87-1.60 (m, 5H), 1.23 (s, 3H) | |
| 341 | ¹H NMR (400 MHz, DMSO-d6) 3:2 mixture of diastereomers, | 578.1 |
| | Major: δ 8.17 (s, 1H), 8.12-7.96 (m, 2H), 7.91 (d, J = 8.2 Hz, | |
| | 1H), 7.40 (s, 1H), 7.34 (t, J = 8.0 Hz, 1H), 6.79 (d, J = 7.7 Hz, | |
| | 1H), 5.15-5.00 (m, 2H), 3.84 (s, 2H), 3.56-3.15 (m, 7H), | |
| | 3.11-2.89 (m, 2H), 2.73 (s, 3H), 2.38-2.08 (m, 3H), 2.02- | |
| | 1.89 (m, 1H), 1.17 (s, J = 27.2 Hz, 3H). | |
| 342 | ¹H NMR (400 MHz, DMSO-d6) δ 8.24 (s, 1H), 8.15 (s, 1H), | 581.1 |
| | 8.05 (s, 2H), 8.02 (s, 1H), 6.51 (s, 1H), 5.17 (s, 2H), 3.83 (s, | |
| | 2H), 3.23 (s, 2H), 2.94 (s, 3H), 2.58-2.51 (m, 3H), 2.33 (dd, | |
| | J = 20.1, 9.2 Hz, 2H), 2.21-2.10 (m, 1H), 2.03-1.93 (m, | |
| | 3H), 1.85 (dd, J = 12.3, 7.0 Hz, 1H), 1.77-1.62 (m, 4H), 1.23 | |
| | (s, 3H), 0.87 (d, J = 6.6 Hz, 6H). | |
| 343 | ¹H NMR (400 MHz, DMSO-d6) δ 8.17 (s, 1H), 8.08-8.02 (m, | 567.2 |
| | 3H), 6.55 (s, 1H), 5.19 (s, 2H), 3.86 (s, 2H), 3.22 (s, 2H), 2.93 | |
| | (s, 3H), 2.61 (t, J = 7.5 Hz, 2H), 2.37-2.29 (m, 3H), 2.21- | |
| | 2.11 (m, 1H), 2.07-1.98 (m, 2H), 1.88-1.58 (m, 8H), 1.26 | |
| | (s, 3H), 0.90 (t, J = 7.3 Hz, 3H). | |
| 344 | ¹H NMR (400 MHz, DMSO-d6) δ 8.34 (s, 1H), 8.15 (s, 1H), | 574.2 |
| | 8.03 (s, 1H), 7.99 (s, 1H), 7.89 (d, J = 8.3 Hz, 1H), 7.38 (s, | |
| | 1H), 7.32 (t, J = 8.0 Hz, 1H), 6.77 (d, J = 7.7 Hz, 1H), 5.07 (s, | |
| | 2H), 3.84 (s, 2H), 3.21 (t, J = 18.6 Hz, 6H), 3.07-2.89 (m, 2H), | |
| | 2.71 (s, 3H), 1.72-1.48 (m, 6H), 1.44-1.34 (m, 2H), 1.16 (s, 3H). | |
| 345 | ¹H NMR (400 MHz, DMSO-d6) δ 8.14-8.10 (m, 2H), 8.05 (s, | 615.2 |
| | 1H), 8.02 (s, 1H), 7.35-7.17 (m, 5H), 6.66 (s, 1H), 5.19 (s, | |
| | 2H), 4.00 (s, 4H), 3.20 (s, 2H), 2.80 (s, 3H), 2.54-2.52 (m, | |
| | 1H), 2.34-2.10 (m, 5H), 1.84-1.73 (m, 5H), 1.38-1.34 (m, 3H). | |
| 346 | ¹H NMR (400 MHz, DMSO-d6) δ 8.14-8.02 (m, 3H), 8.02 (s, | 581.2 |
| | 1H), 6.58 (s, 1H), 5.20 (s, 2H), 4.07 (s, 2H), 3.21 (s, 2H), 2.93 | |
| | (s, 3H), 2.63 (t, J = 7.6 Hz, 2H), 2.53-2.50 (m, 2H), 2.36-2.08 | |
| | (m, 5H), 1.85-1.75 (m, 5H), 1.63-1.52 (m, 2H), 1.47-1.22 | |
| | (m, 5H), 0.90 (t, J = 7.3 Hz, 3H). | |
| 347 | ¹H NMR (400 MHz, DMSO-d6) δ 8.48 (s, 1H), 8.06 (s, 1H), | 562.0 |
| | 8.02 (s, 1H), 7.94 (dd, J = 8.2, 1.8 Hz, 1H), 7.52 (s, 1H), 7.43 | |
| | (t, J = 8.0 Hz, 1H), 6.93 (d, J = 7.8 Hz, 1H), 5.32 (d, J = 6.9 | |
| | Hz, 2H), 5.08 (s, 2H), 5.04 (d, J = 7.0 Hz, 2H), 3.84 (s, 2H), | |
| | 3.11 (s, 3H), 2.05-1.93 (m, 2H), 1.77-1.62 (m, 4H), 1.24 | |
| | (s, 3H). | |
| 348 | ¹H NMR (400 MHz, DMSO-d6) δ 8.18 (s, 1H), 7.96 (d, J = 9.2 | 596.2 |
| | Hz, 2H), 7.37 (s, 1H), 6.66 (t, J = 5.7 Hz, 1H), 5.76 (s, 1H), | |
| | 5.10 (s, 2H), 3.79 (s, 2H), 3.13 (s, 2H), 3.03-2.93 (m, 5H), | |
| | 2.53-2.35 (m, 2H), 2.31-2.16 (m, 2H), 2.15-1.56 (m, 10H), | |
| | 1.21 (s, 3H), 0.89 (d, J = 6.8 Hz, 6H). | |
| 349 | ¹H NMR (400 MHz, DMSO-d6) δ 8.31 (s, 1H), 8.20 (s, 1H), | 585.1 |
| | 8.04 (d, J = 12.0 Hz, 2H), 7.94 (d, J = 1.0 Hz, 1H), 6.63 (d, J = | |
| | 1.0 Hz, 1H), 5.22 (s, 2H), 3.83 (s, 2H), 3.24 (s, 2H), 3.11 (q, | |
| | J = 14.0, 6.7 Hz, 2H), 3.08 (s, 3H), 2.49-2.44 (m, 2H), 2.33 | |
| | (dd, J = 20.3, 9.1 Hz, 2H), 2.11 (dd, J = 19.2, 8.2 Hz, 1H), | |
| | 2.04-1.94 (m, 2H), 1.88-1.78 (m, 1H), 1.78-1.61 (m, 4H), | |
| | 1.33 (t, J = 7.3 Hz, 3H), 1.23 (s, 3H). | |
| 350 | ¹H NMR (400 MHz, DMSO-d6) δ 8.55 (s, 1H), 8.39 (s, 1H), | 651.2 |
| | 8.26-8.16 (m, 3H), 8.12-7.95 (m, 5H), 7.58 (p, J = 6.7 Hz, | |
| | 2H), 7.49 (s, 1H), 5.42 (s, 2H), 3.85 (s, 2H), 3.07 (s, 3H), 2.26- | |
| | 2.10 (m, 2H), 2.00 (dd, J = 17.8, 8.2 Hz, 2H), 1.96-1.85 | |
| | (m, 1H), 1.79-1.62 (m, 4H), 1.31-1.22 (m, 6H) | |
| 351 | ¹H NMR (400 MHz, DMSO-d6) δ 8.34 (bs, 1H), 8.24 (s, 1H), | 559.0 |
| | 8.07 (s, 1H), 8.05 (s, 1H), 6.97 (s, 1H), 5.16 (s, 2H), 3.83 (s, | |
| | 2H), 3.27 (s, 2H), 3.17 (s, 3H), 2.38-2.36 (d, J = 11.0 Hz, 3H), | |
| | 2.20-2.08 (m, 1H), 2.03-1.94 (m, 2H), 1.92-1.81 (m, 2H), | |
| | 1.77-1.62 (m, 4H), 1.23 (s, 3H). | |
| 352 | ¹H NMR (400 MHz, DMSO-d6) δ 8.29 (br s, 1H), 8.17 (s, 1H), | 560.1 |
| | 8.03 (s, 1H), 8.00 (s, 1H), 7.90 (d, J = 7.8 Hz, 1H), 7.41 (s, | |
| | 1H), 7.34 (t, J = 7.9 Hz, 1H), 6.78 (d, J = 7.5 Hz, 1H), 5.09 (s, | |
| | 2H), 3.90 (s, 2H), 3.40-3.13 (m, 6H), 3.02 (dd, J = 28.2, 14.3 | |
| | Hz, 2H), 2.72 (s, 3H), 2.35 (s, 2H), 1.09 (s, 3H), 0.32-0.25 | |
| | (m, 2H), 0.25-0.18 (m, 2H). | |
| 353 | ¹H NMR (400 MHz, DMSO-d6); mixture of diasteromers; δ | 574.1 |
| | 8.25 (s, 1H), 8.17 (s, 1H), 8.07 (d, J = 6.4 Hz, 1H), 8.02 (d, J = | |
| | 4.1 Hz, 1H), 7.91 (d, J = 8.1 Hz, 1H), 7.40 (s, 1H), 7.34 (t, J = | |
| | 8.0 Hz, 1H), 6.79 (d, J = 7.7 Hz, 1H), 5.09 (s, 2H), 3.82 (s, | |
| | 2H), 3.35-3.19 (m, 4H), 3.02 (q, J = 14.0 Hz, 2H), 2.73 (s, | |
| | 3H), 2.42-2.31 (m, 1H), 2.22-2.12 (m, 1H), 2.12-1.99 (m, | |
| | 1H), 1.99-1.81 (m, 1H), 1.58 (t, J = 9.9 Hz, 1H), 1.43 (dd, J = | |
| | 12.4, 7.2 Hz, 1H), 1.22 (s, 3H), 1.14-0.99 (m, 3H). | |
| 354 | ¹H NMR (400 MHz, DMSO-d6) δ 8.17 (s, 1H), 8.02 (s, 1H), | 572.1 |
| | 7.99 (s, 1H), 7.90 (d, J = 8.1 Hz, 1H), 7.40 (s, 1H), 7.34 (t, J = | |
| | 8.0 Hz, 1H), 6.78 (d, J = 7.7 Hz, 1H), 5.09 (s, 2H), 3.87 (s, | |
| | 2H), 3.51-3.19 (m, 5H), 3.02 (q, J = 13.9 Hz, 2H), 2.72 (s, | |
| | 3H), 1.52 (s, 6H), 1.14 (s, 3H). | |
| 355 | ¹H NMR (400 MHz, DMSO-d6) δ 8.46 (br s, 1H), 8.15 (s, 1H), | 588.1 |
| | 8.04 (s, 1H), 7.99 (s, 1H), 7.89 (d, J = 8.1 Hz, 1H), 7.38 (s, | |
| | 1H), 7.32 (t, J = 8.0 Hz, 1H), 6.77 (d, J = 7.9 Hz, 1H), 5.07 (s, | |
| | 2H), 3.81 (s, 2H), 3.49-3.15 (m, 6H), 3.00 (q, J = 13.9 Hz, | |
| | 2H), 2.71 (s, 3H), 1.82 (dd, J = 19.8, 9.5 Hz, 1H), 1.63-1.52 | |
| | (m, 1H), 1.45 (dd, J = 18.8, 9.6 Hz, 1H), 1.40-1.28 (m, 1H), | |
| | 1.15 (s, 3H), 1.14 (s, 3H), 1.00 (s, 3H). | |
| 356 | ¹H NMR (400 MHz, DMSO-d6) δ 8.17 (s, 1H), 8.04 (s, 1H), | 558.1 |
| | 8.00 (s, 1H), 7.90 (dd, J = 8.1, 1.7 Hz, 1H), 7.40 (t, J = 1.8 Hz, | |
| | 1H), 7.34 (t, J = 8.0 Hz, 1H), 6.78 (d, J = 7.8 Hz, 1H), 5.09 (s, | |
| | 2H), 3.90 (s, 2H), 3.30-3.21 (m, 4H), 3.08-2.96 (m, 2H), | |
| | 2.72 (s, 3H), 2.30 (s, 1H), 1.66 (s, 6H). | |
| 357 | ¹H NMR (400 MHz, DMSO-d6) δ 8.27 (br s, 2H), 8.15 (s, 1H), | 606.1 |
| | 8.01 (s, 1H), 7.97 (s, 1H), 7.89 (dd, J = 8.1, 1.6 Hz, 1H), 7.38 | |
| | (t, J = 1.8 Hz, 1H), 7.32 (t, J = 8.0 Hz, 1H), 6.77 (d, J = 7.9 | |
| | Hz, 1H), 5.07 (s, 2H), 4.22-4.05 (m, 1H), 3.97 (dd, J = 8.0, | |
| | 6.3 Hz, 1H), 3.91 (s, 2H), 3.58 (dd, J = 8.0, 6.5 Hz, 1H), 3.35- | |
| | 3.19 (m, 4H), 3.00 (q, J = 14.1 Hz, 2H), 2.70 (s, 3H), 2.65- | |
| | 2.51 (m, 2H), 1.28 (s, 3H), 1.24 (s, 3H). | |
| 358 | ¹H NMR (400 MHz, DMSO-d6) δ 8.23 (br s, 2H), 8.17 (s, 1H), | 576.1 |
| | 8.04 (s, 1H), 7.99 (s, 1H), 7.91 (dd, J = 8.1, 1.6 Hz, 1H), 7.40 | |
| | (s, 1H), 7.34 (t, J = 8.0 Hz, 1H), 6.79 (d, J = 7.7 Hz, 1H), 5.09 | |
| | (s, 2H), 3.90 (s, 2H), 3.78-3.70 (m, 1H), 3.70-3.62 (m, 1H), | |
| | 3.59 (dd, J = 15.3, 7.5 Hz, 1H), 3.38 (dd, J = 8.4, 5.9 Hz, 1H), | |
| | 3.36-3.21 (m, 5H), 3.02 (q, J = 14.1 Hz, 2H), 2.72 (s, 3H), | |
| | 2.48-2.39 (m, 1H), 2.39-2.26 (m, 1H), 2.00-1.85 (m, 1H), | |
| | 1.60-1.42 (m, 1H). | |
| 359 | ¹H NMR (400 MHz, DMSO-d6) δ 8.20 (s, 1H), 8.06 (s, 1H), | 569.6 |
| | 8.03 (s, 1H), 7.82 (s, 1H), 6.17 (s, 1H), 5.19 (s, 2H), 4.29 (q, | |
| | J = 7.0 Hz, 2H), 3.83 (s, 2H), 3.22 (s, 2H), 3.08 (s, 3H), 2.48- | |
| | 2.43 (m, 2H), 2.37-2.27 (m, 2H), 2.17-1.92 (m, 3H), 1.88- | |
| | 1.79 (m, 1H), 1.79-1.61 (m, 4H), 1.33 (t, J = 7.0 Hz, 3H), | |
| | 1.23 (s, 3H) | |
| 360 | ¹H NMR (400 MHz, DMSO-d6) δ 8.17 (s, 1H), 8.01 (d, J = 4.5 | 631.1 |
| | Hz, 2H), 7.78 (s, 1H), 7.42 (d, J = 7.4 Hz, 2H), 7.35 (t, J = 7.5 | |
| | Hz, 2H), 7.28 (m, 1H), 6.22 (s, 1H), 5.35 (s, 2H), 5.08 (s, 2H), | |
| | 3.80 (s, 2H), 3.20 (s, 2H), 3.00 (s, 3H), 2.50-2.41 (m, 3H), | |
| | 2.30 (dd, J = 20.2, 9.1 Hz, 2H), 2.09 (m, 1H), 2.02-1.91 (m, | |
| | 2H), 1.87-1.75 (m, 1H), 1.75-1.59 (m, 4H), 1.21 (s, 3H) | |
| 361 | ¹H NMR (400 MHz, DMSO-d6) δ 8.18 (s, 1H), 8.11-8.02 (m, | 553.2 |
| | 3H), 6.57 (s, 1H), 5.20 (s, 2H), 3.87 (s, 2H), 3.22 (s, 2H), 2.94 | |
| | (s, 3H), 2.66 (q, J = 7.6 Hz, 2H), 2.38-2.30 (m, 3H), 2.18-2.11 | |
| | (m, 1H), 2.09-1.98 (m, 2H), 1.89-1.65 (m, 6H), 1.27 (s, | |
| | 3H), 1.17 (t, J = 7.6 Hz, 3H) | |
| 362 | ¹H NMR (400 MHz, DMSO-d6) δ 8.40 (s, 1H), 8.19 (s, 1H), | 565.1 |
| | 8.05-7.95 (m, 3H), 6.67 (d, J = 1.1 Hz, 1H), 5.14 (s, 2H), | |
| | 3.82 (s, 2H), 3.22 (s, 2H), 3.02 (s, 3H), 2.38-2.30 (m, 3H), | |
| | 2.17-1.63 (m, 10H), 1.23 (s, 3H), 0.96-0.88 (m, 4H) | |
| 363 | ¹H NMR (400 MHz, dmso) δ 8.19 (br s, 1H), 8.17 (s, 1H), 8.05 | 562.1 |
| | (s, 1H), 8.01 (s, 1H), 7.90 (dd, J = 8.2, 1.3 Hz, 1H), 7.41 (t, J = | |
| | 1.6 Hz, 1H), 7.34 (t, J = 8.0 Hz, 1H), 6.78 (d, J = 7.7 Hz, | |
| | 1H), 5.09 (s, 2H), 3.92 (s, 2H), 3.33-3.21 (m, 4H), 3.09- | |
| | 2.95 (m, 2H), 2.72 (s, 3H), 2.22 (s, 2H), 0.88 (s, 9H) | |
| 364 | 1H NMR (400 MHz, DMSO-d6) δ 8.22 (br s, 1H), 8.15 (s, 1H), | 560.3 |
| | 8.02 (s, 1H), 7.98 (s, 1H), 7.89 (dd, J = 8.2, 1.8 Hz, 1H), 7.38 | |
| | (t, J = 1.6 Hz, 1H), 7.32 (t, J = 8.0 Hz, 1H), 6.77 (d, J = 7.7 | |
| | Hz, 1H), 5.07 (s, 2H), 3.86 (s, 2H), 3.26-3.19 (m, 4H), 3.06- | |
| | 2.94 (m, 3H), 2.71 (s, 3H), 1.74-1.56 (m, 4H), 1.51-1.28 (m, 4H) | |
| 365 | ¹H NMR (400 MHz, DMSO-d6) δ 8.19 (s, 1H), 8.00 (s, 1H), | 612.2 |
| | 7.94 (s, 1H), 7.83 (s, 1H), 6.18 (s, 1H), 5.21 (s, 2H), 4.21 (d, | |
| | J = 14.4 Hz, 1H), 3.86 (s, 3H), 3.39 (d, J = 14.4 Hz, 1H), 3.22 | |
| | (s, 2H), 3.07 (s, 3H), 2.625-2.59 (m, 2H), 2.46-2.45 (m, 1H), | |
| | 2.36-2.19 (m, 6H), 2.17-2.05 (m, 4H), 1.99-1.91 (m, 2H), | |
| | 1.86-1.81 (m, 1H), 0.94-0.87 (m, 6H). | |
| 366 | ¹H NMR (400 MHz, DMSO-d6) δ 8.17 (s, 1H), 8.00 (s, 1H), | 588.1 |
| | 7.96 (s, 1H), 7.91 (dd, J = 8.2, 1.9 Hz, 1H), 7.40 (ap s, 1H), | |
| | 7.34 (t, J = 8.0 Hz, 1H), 6.79 (d, J = 7.7 Hz, 1H), 5.08 (s, 2H), | |
| | 4.52 (s, 2H), 4.45 (s, 2H), 3.78 (s, 2H), 3.37-3.18 (m, 5H), 3.10- | |
| | 2.91 (m, 4H), 2.72 (s, 3H), 2.41-2.30 (m, 2H), 1.92-1.80 (m, 2H). | |
| 367 | ¹H NMR (400 MHz, DMSO-d6) δ 8.15 (s, 1H), 7.98 (s, 1H), | 576.1 |
| | 7.95 (s, 1H), 7.89 (dd, J = 8.1, 1.6 Hz, 1H), 7.39-7.36 (m, | |
| | 1H), 7.32 (t, J = 8.0 Hz, 1H), 6.76 (d, J = 7.7 Hz, 1H), 5.06 (s, | |
| | 2H), 3.96 (s, 2H), 3.46-3.12 (m, 10H), 3.08-2.89 (m, 2H), | |
| | 2.71 (s, 3H), 0.56-0.48 (m, 2H), 0.48-0.39 (m, 2H). | |
| 368 | ¹H NMR (400 MHz, DMSO-d6) δ 8.17 (s, 1H), 8.03 (s, 1H), | 562.0 |
| | 7.99 (s, 1H), 7.91 (dd, J = 8.1, 1.8 Hz, 1H), 7.40 (ap s, 1H), | |
| | 7.34 (t, J = 8.0 Hz, 1H), 6.79 (d, J = 7.7 Hz, 1H), 5.09 (s, 2H), | |
| | 4.61 (dd, J = 7.6, 5.9 Hz, 2H), 4.24 (t, J = 5.9 Hz, 2H), 3.89 | |
| | (s, 2H), 3.36-3.18 (m, 4H), 3.08-2.93 (m, 3H), 2.80-2.74 | |
| | (m, 2H), 2.72 (s, 3H). | |
| 369 | ¹H NMR (400 MHz, DMSO-d6) δ 8.22 (s, 1H), 8.17 (s, 1H), | 574.1 |
| | 8.06 (s, 1H), 8.02 (s, 1H), 7.91 (dd, J = 8.2, 1.8 Hz, 1H), 7.40 | |
| | (s, 1H), 7.34 (t, J = 8.0 Hz, 1H), 6.79 (d, J = 7.9 Hz, 1H), 5.09 | |
| | (s, 2H), 3.75 (s, 2H), 3.58-3.13 (m, 6H), 3.11-2.95 (m, 2H), | |
| | 2.73 (s, 3H), 1.95-1.83 (m, 2H), 1.82-1.67 (m, 3H), 1.67- | |
| | 1.52 (m, 3H), 0.82 (t, J = 7.3 Hz, 3H). | |
| 370 | ¹H NMR (400 MHz, DMSO-d6) δ 8.26 (br s, 1H), 8.17 (s, 1H), | 548.1 |
| | 8.05 (s, 1H), 8.02 (s, 1H), 7.91 (dd, J = 8.1, 1.6 Hz, 1H), 7.40 | |
| | (t, J = 1.8 Hz, 1H), 7.34 (t, J = 8.0 Hz, 1H), 6.79 (d, J = 7.8 | |
| | Hz, 1H), 5.09 (s, 2H), 3.98-3.86 (m, 2H), 3.36-3.16 (m, | |
| | 4H), 3.08-2.96 (m, 2H), 2.72 (s, 3H), 2.49-2.44 (m, 1H), | |
| | 1.53-1.41 (m, 1H), 1.35-1.22 (m, 1H), 1.00 (d, J = 6.3 Hz, | |
| | 3H), 0.85 (t, J = 7.4 Hz, 3H) | |
| 371 | ¹H NMR (400 MHz, DMSO-d6) δ 8.28 (s, 1H), 8.17 (s, 1H), | 548.1 |
| | 8.05 (s, 1H), 8.01 (s, 1H), 7.91 (dd, J = 7.9, 1.7 Hz, 1H), 7.40 | |
| | (t, J = 1.8 Hz, 1H), 7.34 (t, J = 8.0 Hz, 1H), 6.79 (d, J = 7.8 | |
| | Hz, 1H), 5.09 (s, 2H), 3.97-3.84 (m, 2H), 3.36-3.16 (m, | |
| | 4H), 3.10-2.95 (m, 3H), 2.72 (s, 3H), 2.49-2.42 (m, 1H), | |
| | 1.52-1.40 (m, 1H), 1.34-1.22 (m, 1H), 0.99 (d, J = 6.3 Hz, | |
| | 3H), 0.85 (t, J = 7.4 Hz, 3H). | |
| 372 | ¹H NMR (400 MHz, DMSO-d6) δ 8.17 (s, 1H), 8.05 (s, 1H), | 566.0 |
| | 8.00 (s, 1H), 7.91 (dd, J = 8.1, 1.9 Hz, 1H), 7.39 (t, J = 1.7 Hz, | |
| | 1H), 7.34 (t, J = 8.0 Hz, 1H), 6.79 (d, J = 7.9 Hz, 1H), 5.09 (s, | |
| | 2H), 4.24 (d, J = 48.0 Hz, 2H), 3.91 (s, 2H), 3.30-3.22 (m, | |
| | 4H), 3.09-2.96 (m, 2H), 2.73 (s, 3H), 1.09 (d, J = 1.9 Hz, 5H) | |
| 373 | ¹H NMR (400 MHz, DMSO-d6) δ 8.15 (s, 1H), 8.00 (d, J = 9.1 | 578.1 |
| | Hz, 2H), 7.88 (d, J = 9.6 Hz, 1H), 7.39 (s, 1H), 7.32 (t, J = 8.0 | |
| | Hz, 1H), 6.76 (d, J = 7.9 Hz, 1H), 5.07 (s, 2H), 3.91 (s, 2H), | |
| | 3.28-3.13 (m, 5H), 3.07-2.93 (m, 5H), 2.70 (s, 3H), 2.40 | |
| | (s, 2H), 1.09 (s, 6H) | |
| 374 | ¹H NMR (400 MHz, CD₃CN) δ 8.00 (s, 1H), 7.94-7.84 (m, | 643.2 |
| | 3H), 7.36-7.32 (m, 2), 6.84-6.79 (m, 1H), 4.95 (s, 2H), 4.06 | |
| | (s, 2H), 3.47-3.22 (m, 5H), 3.08-2.95 (m, 3H), 2.84 (dd, J = | |
| | 11.9, 3.3 Hz, 1H), 2.69 (s, 3H), 2.56-2.41 (m, 3H), 2.21 (s, | |
| | 3H), 2.13-2.10 (m, 1H) | |
| 375 | ¹H NMR (400 MHz, DMSO-d6) δ 8.25 (s, 1H), 8.01 (s, 1H), | 619.1 |
| | 7.96 (s, 1H), 7.92 (d, J = 0.6 Hz, 1H), 6.42 (d, J = 0.6 Hz, 1H), | |
| | 5.19 (s, 2H), 4.91 (d, J = 6.2 Hz, 2H), 4.83 (d, J = 6.2 Hz, 2H), | |
| | 4.31 (q, J = 7.0 Hz, 2H), 3.75 (AB q, 2H), 3.56 (s, 2H), 2.82- | |
| | 2.66 (m, 2H), 2.39-2.25 (m, 1H), 2.21-1.84 (m, 4H), 1.34 | |
| | (t, J = 7.0 Hz, 3H), 0.93 (d, J = 6.4 Hz, 3H). | |
| 376 | ¹H NMR (400 MHz, dmso) δ 9.21-9.18 (m, 1H), 8.20 (s, 1H), | 654.2 |
| | 7.94 (d, J = 19.8 Hz, 4H), 7.84 (d, J = 8.2 Hz, 1H), 7.46 (t, J = | |
| | 8.0 Hz, 1H), 7.22 (d, J = 7.7 Hz, 1H), 6.48 (d, J = 55.4 Hz, | |
| | 1H), 5.32-5.04 (m, 3H), 4.84 (d, J = 11.3 Hz, 1H), 4.60 (d, J = | |
| | 11.3 Hz, 1H), 4.21 (d, J = 14.4 Hz, 1H), 3.81 (ap s, 2H), 3.37 | |
| | (d, J = 14.4 Hz, 1H), 2.61 (t, J = 12.6 Hz, 2H), 2.37-2.17 (m, | |
| | 3H), 2.05-1.83 (m, 2H), 0.92 (d, J = 6.6 Hz, 3H), 0.87 (d, J = | |
| | 6.6 Hz, 3H). | |
| 377 | ¹H NMR (400 MHz, DMSO-d6) δ 8.15 (s, 1H), 8.07-7.89 (m, | 654.2 |
| | 4H), 7.86 (d, J = 8.0 Hz, 1H), 7.48 (t, J = 8.0 Hz, 1H), 7.24 (d, | |
| | J = 7.5 Hz, 1H), 6.50 (d, J = 55.4 Hz, 1H), 5.27-5.13 (m, 3H), | |
| | 4.86 (d, J = 11.3 Hz, 1H), 4.62 (d, J = 11.2 Hz, 1H), 4.23 (d, | |
| | J = 14.4 Hz, 1H), 3.83 (ap s, 2H), 3.38 (d, J = 14.4 Hz, 1H), | |
| | 2.63 (t, J = 9.8 Hz, 2H), 2.36-2.18 (m, 3H), 2.16 (s, 3H), 2.04- | |
| | 1.85 (m, 3H), 0.93 (d, J = 6.6 Hz, 3H), 0.89 (d, J = 6.6 Hz, 3H) | |
| 378 | ¹H NMR (400 MHz, DMSO-d6) δ 8.17 (s, 1H), 8.00 (d, J = 9.8 | 594.1 |
| | Hz, 2H), 7.37 (s, 1H), 6.60 (t, J = 5.6 Hz, 1H), 5.78 (s, 1H), | |
| | 5.12 (s, 2H), 3.82 (s, 2H), 3.14 (s, 2H), 3.05 (t, J = 6.2 Hz, | |
| | 2H), 3.02 (s, 3H), 2.43 (m, 2H), 2.25 (m, 2H), 2.13-1.90 (m, | |
| | 3H), 1.85-1.61 (m, 5H), 1.23 (s, 3H), 1.08-0.92 (m, 1H), | |
| | 0.47-0.32 (m, 2H), 0.24-0.12 (m, 2H). | |
| 379 | ¹H NMR (400 MHz, DMSO-d6) δ 10.53 (s, 1H), 8.15 (s, 1H), | 651.1 |
| | 7.98 (s, 1H), 7.91 (d, J = 7.9 Hz, 2H), 7.72 (d, J = 8.6 Hz, 1H), | |
| | 6.65 (dd, J = 17.0, 10.2 Hz, 1H), 6.28 (dd, J = 17.0, 1.8 Hz, | |
| | 1H), 5.77 (dd, J = 10.2, 1.8 Hz, 1H), 5.18 (s, 2H), 4.19 (d, J = | |
| | 14.4 Hz, 1H), 3.36 (d, J = 14.4 Hz, 2H), 3.22 (s, 2H), 3.03 (s, | |
| | 3H), 2.58 (dd, J = 24.8, 14.7 Hz, 2H), 2.37-2.16 (m, 6H), | |
| | 2.15-2.03 (m, 5H), 1.88 (ddd, J = 19.9, 14.8, 7.6 Hz, 3H), | |
| | 0.91 (d, J = 6.6 Hz, 3H), 0.87 (d, J = 6.6 Hz, 3H. | |
| 380 | ¹H NMR (400 MHz, DMSO-d6) δ 8.44 (d, J = 5.2 Hz, 2H), 8.35 | 631.9 |
| | (s, 1H), 8.19 (s, 1H), 7.95 (s, 2H), 7.40-7.30 (m, 2H), 7.25 | |
| | (d, J = 5.6 Hz, 2H), 5.82 (s, 1H), 4.80 (s, 2H), 4.39 (d, J = 5.7 | |
| | Hz, 2H), 3.77 (s, 2H), 3.12 (s, 2H), 2.92 (s, 3H), 2.45-2.34 | |
| | (m, 1H), 2.32-2.15 (m, 2H), 2.15-1.99 (m, 1H), 1.99-1.84 | |
| | (m, 2H), 1.83-1.60 (m, 6H), 1.19 (s, 4H) | |
| 381 | ¹H NMR (400 MHz, DMSO-d6) δ 8.26 (s, 1H), 8.12-8.04 (m, | 514.3 |
| | 2H), 7.91 (d, J = 1.0 Hz, 1H), 7.80 (t, J = 7.7 Hz, 1H), 6.40 (d, | |
| | J = 0.9 Hz, 1H), 5.31-5.25 (m, 1H), 5.23 (s, 2H), 4.90 (d, J = | |
| | 6.2 Hz, 2H), 4.82 (d, J = 6.2 Hz, 2H), 3.55 (s, 2H), 3.30 (s, | |
| | 3H), 2.05-1.92 (m, 2H), 1.80-1.67 (m, 4H), 1.67-1.55 (m, 2H). | |
| 382 | 1H NMR (400 MHz, DMSO-d6) δ 8.25 (s, 1H), 8.04 (dd, J = | 525.2 |
| | 12.0, 7.7 Hz, 2H), 7.78 (t, J = 7.7 Hz, 1H), 7.43 (d, J = 1.1 Hz, | |
| | 1H), 6.79 (d, J = 5.7 Hz, 1H), 5.98 (s, 1H), 5.19 (s, 2H), 4.90 | |
| | (d, J = 6.0 Hz, 2H), 4.79 (dd, J = 6.0, 2.7 Hz, 2H), 4.02 (s, | |
| | 1H), 3.49 (s, 2H), 3.24 (s, 3H), 2.57 (d, J = 6.7 Hz, 2H), 2.05 | |
| | (t, J = 9.6 Hz, 1H), 1.70-1.51 (m, 2H), 1.32 (ddd, J = 22.7, | |
| | 10.4, 7.3 Hz, 2H), 1.02 (dd, J = 9.8, 6.7 Hz, 1H). | |
| 383 | ¹H NMR (400 MHz, methanol-d₄) δ 8.33 (s, 1H), 8.03 (s, 1H), | 598.1 |
| | 7.93 (s, 1H), 7.80 (d, J = 8.0 Hz, 1H), 7.66 (s, 1H), 7.47 (t, J = | |
| | 8.0 Hz, 1H), 7.05 (d, J = 8.0 Hz, 1H), 5.09 (s, 2H), 3.87 (s, | |
| | 2H), 3.75-3.70 (m, 2H), 3.37-3.34 (m, 2H), 3.30-3.24 (m, | |
| | 2H), 3.24-3.21 (m, 2H), 2.96 (s, 3H), 1.49 (s, 3H). | |
| 384 | ¹H NMR (400 MHz, methanol-d₄) δ 8.16 (s, 1H), 8.08 (s, 1H), | 506.1 |
| | 7.99 (s, 1H), 7.73 (d, J = 8.0 Hz, 1H), 7.47 (s, 1H), 7.38 (d, J = | |
| | 8.0 Hz, 1H), 6.81 (d, J = 7.6 Hz, 1H), 5.06 (s, 2H), 4.27- | |
| | 4.23 (m, 1H), 3.35 (s, 2H), 3.29-3.23 (m, 2H), 3.11-2.97 | |
| | (m, 2H), 2.76 (s, 3H), 1.44 (d, J = 6.8 Hz, 3H). | |
| 385 | ¹H NMR (400 MHz, methanol-d₄) δ 8.16 (s, 1H), 8.08 (s, 1H), | 506.1 |
| | 7.99 (s, 1H), 7.73 (d, J = 7.6 Hz, 1H), 7.47 (s, 1H), 7.38 (d, J = | |
| | 8.0 Hz, 1H), 6.81 (d, J = 7.2 Hz, 1H), 5.06 (s, 2H), 4.29- | |
| | 4.23 (m, 1H), 3.35 (s, 2H), 3.29-3.23 (m, 2H), 3.11-2.97 | |
| | (m, 2H), 2.76 (s, 3H), 1.44 (d, J = 6.8 Hz, 3H). | |
| 386 | ¹H NMR (400 MHz, methanol-d₄) δ 8.22 (s, 1H), 8.18 (s, 1H), | 474.2 |
| | 8.12 (s, 1H), 7.71 (d, J = 8.4 Hz, 1H), 7.46 (s, 1H), 7.41 (d, J = | |
| | 8.0 Hz, 1H), 6.92 (d, J = 7.6 Hz, 1H), 6.14 (d, J = 44.4 Hz, | |
| | 1H), 5.20-5.08 (m, 2H), 4.37 (s, 2H), 3.01-2.90 (m, 1H), | |
| | 2.90-2.70 (m, 4H), 2.64-2.49 (m, 2H), 2.28-2.24 (m, 1H), | |
| | 2.01-1.96 (m, 1H). | |
| 387 | ¹H NMR (400 MHz, methanol-d₄) δ 8.22 (s, 1H), 8.18 (s, 1H), | 474.2 |
| | 8.12 (s, 1H), 7.71 (d, J = 8.0 Hz, 1H), 7.46 (s, 1H), 7.41 (d, J = | |
| | 8.0 Hz, 1H), 6.92 (d, J = 8.0 Hz, 1H), 6.14 (d, J = 44.4 Hz, | |
| | 1H), 5.20-5.08 (m, 2H), 4.37 (s, 2H), 3.01-2.90 (m, 1H), | |
| | 2.90-2.70 (m, 4H), 2.64-2.49 (m, 2H), 2.28-2.24 (m, 1H), | |
| | 2.01-1.96 (m, 1H). | |
| 388 | ¹H NMR (400 MHz, methanol-d₄) δ 8.25 (s, 1H), 8.18-8.09 | 649.3 |
| | (m, 1H), 8.01-7.87 (m, 1H), 7.78 (d, J = 8.4 Hz, 1H), 7.60- | |
| | 7.58 (m, 1H), 7.46-7.41 (m, 1H), 7.02-6.99 (m, 1H), 6.07 | |
| | (d, J = 44.8 Hz, 1H), 5.09 (s, 2H), 4.55-4.53 (m, 1H), 3.70- | |
| | 3.50 (m, 2H), 3.21-3.08 (m, 3H), 2.94 (s, 3H), 2.70-2.50 (m, | |
| | 3H), 2.35-2.00 (m, 7H), 1.52-1.43 (m, 3H), 1.05-0.94 (m, 6H). | |
| 389 | ¹H NMR (400 MHz, methanol-d₄) δ 8.28 (s, 1H), 8.09 (s, 1H), | 530.2 |
| | 8.01 (s, 1H), 7.80 (d, J = 8.0 Hz, 1H), 7.53 (s, 1H), 7.43 (t, J = | |
| | 8.0 Hz, 1H), 6.92 (d, J = 8.0 Hz, 1H), 6.31 (d, J = 45.2 Hz, | |
| | 1H), 5.48 (d, J = 6.4 Hz, 1H), 5.31 (d, J = 6.4 Hz, 1H), 5.30- | |
| | 5.20 (m, 1H), 5.10 (s, 2H), 5.04-5.02 (m, 1H), 4.19 (t, J = | |
| | 7.2 Hz, 1H), 3.11 (s, 3H), 1.67 (t, J = 7.2 Hz, 2H), 0.70-0.61 | |
| | (m, 1H), 0.48-0.45 (m, 1H), 0.45-0.30 (m, 1H), 0.12-0.10 | |
| | (m, 1H), −0.05-−0.06 (m, 1H). | |
| 390 | ¹H NMR (400 MHz, methanol-d₄) δ 8.28 (s, 1H), 8.09 (s, 1H), | 530.1 |
| | 8.02 (s, 1H), 7.80 (d, J = 8.0 Hz, 1H), 7.53 (s, 1H), 7.43 (t, J = | |
| | 8.0 Hz, 1H), 6.92 (d, J = 8.0 Hz, 1H), 6.31 (d, J = 45.2 Hz, | |
| | 1H), 5.48 (d, J = 6.4 Hz, 1H), 5.31 (d, J = 6.4 Hz, 1H), 5.30- | |
| | 5.20 (m, 1H), 5.10 (s, 2H), 5.04-5.02 (m, 1H), 4.19 (t, J = | |
| | 7.2 Hz, 1H), 3.12 (s, 3H), 1.67 (t, J = 7.2 Hz, 2H), 0.70-0.61 | |
| | (m, 1H), 0.48-0.45 (m, 1H), 0.40-0.37 (m, 1H), 0.12-0.10 | |
| | (m, 1H), −0.03-−0.06 (m, 1H). | |
| 391 | ¹H NMR (400 MHz, methanol-d₄) δ 8.28 (s, 1H), 8.03 (s, 1H), | 530.1 |
| | 7.96 (s, 1H), 7.79 (d, J = 8.0 Hz, 1H), 7.52 (s, 1H), 7.43 (t, J = | |
| | 8.0 Hz, 1H), 6.92 (d, J = 8.0 Hz, 1H), 6.31 (d, J = 45.2 Hz, | |
| | 1H), 5.48 (d, J = 6.4 Hz, 1H), 5.31 (d, J = 6.4 Hz, 1H), 5.23- | |
| | 5.20 (m, 1H), 5.08 (s, 2H), 5.04-5.01 (m, 1H), 3.97 (d, J = | |
| | 9.6 Hz, 1H), 3.11 (s, 3H), 2.64-2.55 (m, 1H), 2.25-2.18 (m, | |
| | 1H), 2.10-1.70 (m, 5H). | |
| 392 | ¹H NMR (400 MHz, methanol-d₄) δ 8.28 (s, 1H), 8.03 (s, 1H), | 530.1 |
| | 7.96 (s, 1H), 7.79 (d, J = 8.0 Hz, 1H), 7.52 (s, 1H), 7.43 (t, J = | |
| | 8.0 Hz, 1H), 6.92 (d, J = 8.0 Hz, 1H), 6.31 (d, J = 45.6 Hz, | |
| | 1H), 5.48 (d, J = 6.4 Hz, 1H), 5.31 (d, J = 6.4 Hz, 1H), 5.23- | |
| | 5.20 (m, 1H), 5.08 (s, 2H), 5.04-5.01 (m, 1H), 3.98 (d, J = | |
| | 9.6 Hz, 1H), 3.11 (s, 3H), 2.64-2.58 (m, 1H), 2.25-2.18 (m, | |
| | 1H), 2.10-1.70 (m, 5H). | |
| 393 | ¹H NMR (400 MHz, methanol-d₄) δ 8.49 (s, 1H), 8.25 (s, 1H), | 510.1 |
| | 8.17 (s, 1H), 8.10 (s, 1H), 7.78 (d, J = 8.4 Hz, 1H), 7.61 (s, | |
| | 1H), 7.45 (t, J = 8.0 Hz, 1H), 7.04 (d, J = 7.6 Hz, 1H), 6.07 (d, | |
| | J = 44.8 Hz, 1H), 5.21-5.11 (m, 2H), 4.33 (s, 2H), 3.66- | |
| | 3.58 (m, 1H), 3.54-3.47 (m, 1H), 3.22-3.07 (m, 2H), 2.96 (s, 3H). | |
| 394 | ¹H NMR (400 MHz, methanol-d₄) δ 8.51 (br s, 1H), 8.25 (s, | 510.1 |
| | 1H), 8.17 (s, 1H), 8.10 (s, 1H), 7.78 (d, J = 8.4 Hz, 1H), 7.61 | |
| | (s, 1H), 7.45 (t, J = 8.0 Hz, 1H), 7.04 (d, J = 7.6 Hz, 1H), 6.07 | |
| | (d, J = 44.8 Hz, 1H), 5.21-5.10 (m, 2H), 4.32 (s, 2H), 3.66- | |
| | 3.58 (m, 1H), 3.54-3.47 (m, 1H), 3.22-3.07 (m, 2H), 2.96 (s, 3H). | |
| 395 | ¹H NMR (400 MHz, methanol-d₄) δ 8.26 (s, 1H), 8.17 (s, 1H), | 564.2 |
| | 7.99 (s, 1H), 7.79 (d, J = 8.4 Hz, 1H), 7.61 (s, 1H), 7.44 (t, J = | |
| | 8.0 Hz, 1H), 6.99 (d, J = 7.6 Hz, 1H), 6.07 (d, J = 44.4 Hz, | |
| | 1H), 5.16-5.05 (m, 2H), 3.85 (s, 2H), 3.70-3.50 (m, 3H), | |
| | 3.22-3.07 (m, 3H), 2.65-2.50 (m, 4H), 1.90-1.80 (m, 5H). | |
| 396 | ¹H NMR (400 MHz, methanol-d₄) δ 8.25 (s, 1H), 8.15 (s, 1H), | 564.2 |
| | 8.07 (s, 1H), 7.79 (d, J = 7.6 Hz, 1H), 7.61 (s, 1H), 7.45 (t, J = | |
| | 8.0 Hz, 1H), 7.02 (d, J = 8.4 Hz, 1H), 6.07 (d, J = 44.4 Hz, | |
| | 1H), 5.19-5.09 (m, 2H), 4.19 (s, 2H), 3.66-3.50 (m, 2H), | |
| | 3.24-3.11 (m, 2H), 3.09-2.95 (m, 7H), 1.90-1.80 (m, 4H). | |
| 397 | ¹H NMR (400 MHz, methanol-d₄) δ 8.24 (s, 1H), 8.00 (s, 1H), | 550.0 |
| | 7.90 (s, 1H), 7.76 (d, J = 8.0 Hz, 1H), 7.59 (s, 1H), 7.42 (t, J = | |
| | 8.0 Hz, 1H), 6.96 (d, J = 8.0 Hz, 1H), 6.06 (d, J = 44.4 Hz, | |
| | 1H), 5.08 (s, 2H), 3.80 (s, 2H), 3.64-3.48 (m, 2H), 3.36-3.31 | |
| | (m, 4H), 3.30-3.09 (m, 2H), 2.92 (s, 3H), 2.18-2.11 (m, 2H). | |
| 398 | ¹H NMR (400 MHz, methanol-d₄) δ 8.24 (s, 1H), 8.00 (s, 1H), | 550.1 |
| | 7.90 (s, 1H), 7.76 (d, J = 8.0 Hz, 1H), 7.59 (s, 1H), 7.41 (t, J = | |
| | 8.0 Hz, 1H), 6.96 (d, J = 7.6 Hz, 1H), 6.06 (d, J = 44.4 Hz, | |
| | 1H), 5.08 (s, 2H), 3.80 (s, 2H), 3.64-3.48 (m, 2H), 3.36-3.31 | |
| | (m, 4H), 3.30-3.09 (m, 2H), 2.92 (s, 3H), 2.18-2.10 (m, 2H). | |
| 399 | ¹H NMR (400 MHz, methanol-d₄) δ 8.33 (s, 1H), 8.08 (s, 1H), | 528.1 |
| | 8.01 (s, 1H), 7.80 (d, J = 8.4 Hz, 1H), 7.68 (s, 1H), 7.47 (t, J = | |
| | 8.0 Hz, 1H), 7.06 (d, J = 7.6 Hz, 1H), 5.08 (s, 2H), 4.04 (s, | |
| | 2H), 3.93-3.67 (m, 2H), 3.28-3.18 (m, 2H), 2.98 (s, 3H). | |
| 400 | ¹H NMR (400 MHz, methanol-d₄) δ 8.18 (s, 1H), 8.08 (s, 1H), | 492.2 |
| | 8.00 (s, 1H), 7.75 (d, J = 8.4 Hz, 1H), 7.50 (s, 1H), 7.40 (t, J = 8.0 | |
| | Hz, 1H), 6.84 (d, J = 8.0 Hz, 1H), 5.09 (s, 2H), 4.02 (s, 2H), 3.38- | |
| | 3.34 (m, 2H), 3.28-3.28 (m, 2H), 3.11-3.03 (m, 2H), 2.78 (s, 3H). | |
| 401 | ¹H NMR (400 MHz, methanol-d₄) δ 8.18 (s, 1H), 8.08 (s, 1H), | 603.4 |
| | 7.94 (s, 1H), 7.75 (d, J = 8.4 Hz, 1H), 7.48 (s, 1H), 7.40 (t, J = | |
| | 8.0 Hz, 1H), 6.84 (d, J = 8.0 Hz, 1H), 5.08 (s, 2H), 4.29- | |
| | 4.24 (m, 1H), 3.44-3.31 (m, 4H), 3.28-3.25 (m, 1H), 3.07- | |
| | 3.02 (m, 2H), 2.85-2.80 (m, 1H), 2.78 (s, 3H), 2.73-2.69 | |
| | (m, 2H), 2.50-2.44 (m, 1H), 2.44-2.32 (m, 1H), 2.32 (s, | |
| | 3H), 2.30-2.15 (m, 2H), 1.72-1.62 (m, 1H), 1.62-1.58 (m, | |
| | 1H), 0.98 (t, J = 7.2 Hz, 3H). | |
| 402 | ¹H NMR (400 MHz, methanol-d₄) δ 8.18 (s, 1H), 8.11 (s, 1H), | 615.3 |
| | 7.98 (s, 1H), 7.75 (d, J = 9.6 Hz, 1H), 7.48 (s, 1H), 7.40 (t, J = | |
| | 8.0 Hz, 1H), 6.85 (d, J = 7.2 Hz, 1H), 5.08 (s, 2H), 3.37 (s, | |
| | 2H), 3.14-2.93 (m, 7H), 2.78 (s, 3H), 2.71-2.66 (m, 2H), | |
| | 2.33-2.21 (m, 6H), 1.63-1.60 (m, 1H), 0.58-0.54 (m, 3H), | |
| | 0.42-0.39 (m, 1H), 0.20-0.17 (m, 1H). | |
| 403 | ¹H NMR (400 MHz, methanol-d₄) δ 8.18 (s, 1H), 8.06 (s, 1H), | 615.3 |
| | 7.96 (s, 1H), 7.75 (dd, J = 1.2, 8.0 Hz, 1H), 7.48 (s, 1H), 7.40 | |
| | (t, J = 8.0 Hz, 1H), 6.84 (d, J = 7.6 Hz, 1H), 5.09 (s, 2H), 3.80- | |
| | 3.68 (m, 2H), 3.37 (s, 3H), 3.30-3.29 (m, 1H), 3.07-3.02 | |
| | (m, 2H), 2.84-2.78 (m, 2H), 2.78 (s, 3H), 2.77-2.75 (m, | |
| | 1H), 2.47 (s, 3H), 2.36-2.33 (m, 2H), 2.20-2.16 (m, 1H), | |
| | 1.33-1.25 (m, 1H), 0.71-0.69 (m, 2H), 0.48-0.44 (m, 1H), | |
| | 0.36-0.34 (m, 1H), 0.04-0.01 (m, 1H). | |
| 404 | ¹H NMR (400 MHz, methanol-d₄) δ 8.20 (s, 1H), 8.08 (s, 1H), | 626.4 |
| | 7.95 (s, 1H), 7.75 (s, 1H), 6.13 (s, 1H), 5.24 (s, 2H), 4.40- | |
| | 4.31 (m, 3H), 3.34 (s, 3H), 3.05 (s, 3H), 2.84-2.76 (m, 1H), | |
| | 2.76-2.60 (m, 2H), 2.60-2.50 (m, 2H), 2.50-2.44 (m, 2H), | |
| | 2.44-2.26 (m, 8H), 2.25-2.18 (m, 1H), 2.00-1.80 (m, 1H), | |
| | 1.39 (t, J = 7.2 Hz, 3H), 1.03-0.97 (m, 6H). | |
| 405 | ¹H NMR (400 MHz, acetonitrile-d₄) δ 8.02 (s, 1H), 8.01 (s, | 627.2 |
| | 1H), 7.96-7.92 (m, 2H), 7.50 (s, 1H), 7.41 (t, J = 8.0 Hz, 1H), | |
| | 6.96 (d, J = 8.0 Hz, 1H), 6.16 (d, J = 46.0 Hz, 1H), 5.45-5.42 | |
| | (m, 1H), 5.29-5.26 (m, 1H), 5.21-5.18 (m, 1H), 4.99 (s, | |
| | 2H), 4.97-4.94 (m, 1H), 4.26 (d, J = 14.4 Hz, 1H), 3.36 (d, J = | |
| | 14.4 Hz, 1H), 3.13 (s, 3H), 2.86-2.75 (m, 1H), 2.75-2.69 | |
| | (m, 2H), 2.31-2.26 (m, 3H), 2.17 (s, 2H), 1.62-1.60 (m, | |
| | 1H), 0.99 (d, J = 7.2 Hz, 3H), 0.96 (d, J = 6.8 Hz, 3H), 0.44- | |
| | 0.42 (m, 2H), 0.33-0.30 (m, 2H). | |
| 406 | ¹H NMR (400 MHz, CDCl₃) δ 8.06 (s, 1H), 7.91 (s, 1H), 7.85 | 601.6 |
| | (s, 1H), 7.67-7.64 (m, 1H), 7.45 (s, 1H), 7.41-7.36 (m, 1H), | |
| | 6.79 (d, J = 7.6 Hz, 1H), 6.47 (d, J = 46.0 Hz, 1H), 5.33-5.28 | |
| | (m, 2H), 5.25-5.23 (m, 1H), 5.01-4.99 (m, 1H), 4.95-4.91 | |
| | (m, 1H), 3.73 (d, J = 13.6 Hz, 1H), 3.63 (d, J = 14.0 Hz, 1H), | |
| | 3.05-3.00 (m, 4H), 2.79-2.76 (m, 1H), 2.72-2.69 (m, 1H), | |
| | 2.60-2.50 (m, 1H), 2.40-2.37 (m, 4H), 2.19-2.13 (m, 4H), | |
| | 0.93 (t, J = 6.4 Hz, 6H). | |
| 407 | ¹H NMR (400 MHz, CDCl₃) δ 8.08 (s, 1H), 7.86 (s, 1H), 7.84 | 583.3 |
| | (s, 1H), 7.53-7.50 (m, 2H), 7.35-7.30 (m, 1H), 6.61 (d, J = | |
| | 7.6 Hz, 1H), 5.15 (d, J = 6.4 Hz, 2H), 5.08 (d, J = 6.4 Hz, 2H), | |
| | 4.92 (s, 2H), 4.27 (d, J = 14.4 Hz, 1H), 3.60 (s, 2H), 3.24 (d, | |
| | J = 14.0 Hz, 1H), 2.87 (s, 3H), 2.75-2.70 (m, 2H), 2.69- | |
| | 2.67 (m, 1H), 2.33-2.24 (m, 6H), 2.10-1.95 (m, 2H), 0.99 | |
| | (d, J = 6.8 Hz, 3H), 0.96 (d, J = 6.8 Hz, 3H). | |
| 408 | ¹H NMR (400 MHz, DMSO-d6) δ 8.14 (s, 1H), 7.95 (s, 1H), | 638.4 |
| | 7.89 (s, 1H), 6.99 (s, 1H), 6.49 (s, 1H), 5.83 (s, 1H), 5.46 (d, | |
| | J = 8.0 Hz, 1H), 4.97 (s, 2H), 4.21 (d, J = 14.4 Hz, 1H), 3.43- | |
| | 3.40 (m, 1H), 3.12 (s, 2H), 2.77 (s, 3H), 2.70-2.50 (m, 3H), | |
| | 2.44-2.43 (m, 1H), 2.33-2.20 (m, 11H), 2.10-1.75 (m, | |
| | 3H), 1.06 (d, J = 6.4 Hz, 6H), 0.93 (d, J = 6.8 Hz, 3H), 0.88 | |
| | (d, J = 6.8 Hz, 3H). | |
| 409 | ¹H NMR (400 MHz, methanol-d₄) δ 8.21 (s, 1H), 8.06 (s, 1H), | 639.3 |
| | 7.93 (s, 1H), 7.37 (s, 1H), 5.73 (s, 1H), 5.20 (s, 2H), 4.61 (s, | |
| | 2H), 4.33 (d, J = 14.0 Hz, 1H), 3.99-3.93 (m, 1H), 3.03 (s, | |
| | 3H), 2.82-2.71 (m, 3H), 2.54-2.51 (m, 2H), 2.43-2.08 (m, 12H), | |
| | 2.00-1.93 (m, 1H), 1.20 (d, J = 6.4 Hz, 6H), 1.03-0.97 (m, 6H). | |
| 410 | ¹H NMR (400 MHz, acetonitrile-d₄) δ 7.97 (s, 1H), 7.89 (s, | 581.2 |
| | 1H), 7.83 (s, 1H), 7.78 (d, J = 8.0 Hz, 1H), 7.32-7.29 (m, | |
| | 1H), 7.28-7.24 (m, 1H), 6.74 (d, J = 7.6 Hz, 1H), 4.92 (s, | |
| | 2H), 4.24 (d, J = 14.0 Hz, 1H), 3.32 (d, J = 14.4 Hz, 1H), 3.21 | |
| | (s, 2H), 2.70-2.67 (m, 1H), 2.67 (s, 3H), 2.67-2.57 (m, 3H), | |
| | 2.55-2.45 (m, 1H), 2.45-2.30 (m, 2H), 2.25-2.18 (m, 5H), | |
| | 2.17 (s, 3H), 1.98-1.70 (m, 2H), 1.96 (d, J = 6.0 Hz, 3H), | |
| | 0.92 (d, J = 6.4 Hz, 3H). | |
| 411 | ¹H NMR (400 MHz, methanol-d₄) δ 8.20 (s, 1H), 8.09 (s, 1H), | 575.2 |
| | 7.98 (s, 1H), 7.76 (d, J = 1.6 Hz, 1H), 7.50 (t, J = 1.6 Hz, 1H), | |
| | 7.41 (t, J = 8.0 Hz, 1H), 6.87 (d, J = 8.0 Hz, 1H), 5.11 (s, 2H), | |
| | 3.76 (s, 2H), 3.39 (s, 2H), 3.39-3.33 (m, 1H), 3.33-3.29 (m, 1H), | |
| | 3.09-3.04 (m, 2H), 2.80 (s, 3H), 2.75-2.40 (m, 8H), 2.33 (s, 3H). | |
| 412 | ¹H NMR (400 MHz, methanol-d₄) δ 8.18 (s, 1H), 8.08 (s, 1H), | 587.3 |
| | 7.99 (s, 1H), 7.75 (d, J = 8.8 Hz, 1H), 7.48 (s, 1H), 7.40 (t, J = | |
| | 8.0 Hz, 1H), 6.84 (d, J = 7.6 Hz, 1H), 5.08 (s, 2H), 3.90 (s, | |
| | 2H), 3.59 (d, J = 6.4 Hz, 2H), 3.36 (s, 3H), 3.31-3.25 (m, | |
| | 2H), 3.15-3.00 (m, 4H), 2.99-2.95 (m, 2H), 2.77 (s, 3H), | |
| | 2.54 (s, 3H), 2.04 (d, J = 8.4 Hz, 1H). | |
| 413 | ¹H NMR (400 MHz, methanol-d₄) δ 8.40 (br s, 1H), 8.17 (s, | 587.2 |
| | 1H), 8.12 (s, 1H), 7.99 (s, 1H), 7.74 (d, J = 8.0 Hz, 1H), 7.46- | |
| | 7.39 (m, 2H), 6.90 (d, J = 7.6 Hz, 1H), 5.10 (s, 2H), 4.32- | |
| | 4.07 (m, 4H), 3.37-3.33 (m, 4H), 3.31-3.18 (m, 5H), 3.22- | |
| | 3.02 (m, 4H), 2.79 (s, 3H), 2.61 (s, 1H), 2.47-2.43 (m, 1H). | |
| 414 | ¹H NMR (400 MHz, methanol-d₄) δ 8.18 (s, 1H), 8.09 (s, 1H), | 587.3 |
| | 8.00 (s, 1H), 7.77-7.73 (m, 1H), 7.48 (d, J = 2.0 Hz, 1H), | |
| | 7.40 (t, J = 8.0 Hz, 1H), 6.84 (d, J = 8.0 Hz, 1H), 5.08 (s, 2H), | |
| | 3.99-3.86 (m, 2H), 3.40-3.31 (m, 5H), 3.07-2.98 (m, 4H), | |
| | 2.82-2.77 (m, 1H), 2.72 (s, 3H), 2.72-2.71 (m, 1H), 2.70- | |
| | 2.66 (m, 1H), 2.44 (s, 3H), 1.90-1.81 (m, 2H). | |
| 415 | ¹H NMR (400 MHz, DMSO-d6) δ 8.17 (s, 1H), 8.10-8.04 (m, | 534.1 |
| | 1H), 7.93-7.90 (m, 1H), 7.39-7.36 (m, 2H), 6.95-6.64 (m, | |
| | 2H), 5.11-5.00 (m, 2H), 4.00 (s, 1H), 3.75 (s, 1H), 3.69- | |
| | 3.64 (m, 1H), 3.25 (s, 3H), 3.10-2.90 (m, 2H), 2.80-2.50 | |
| | (m, 4H), 1.40-1.30 (m, 3H), 1.08 (s, 3H). | |
| 416 | ¹H NMR (400 MHz, methanol-d₄) δ 8.18 (s, 1H), 8.08 (s, 1H), | 548.2 |
| | 7.98 (s, 1H), 7.75 (d, J = 8.0 Hz, 1H), 7.49 (s, 1H), 7.40 (t, J = | |
| | 8.0 Hz, 1H), 6.85 (d, J = 7.2 Hz, 1H), 5.09 (s, 2H), 3.82 (s, | |
| | 2H), 3.37 (s, 3H), 3.31-3.25 (m, 1H), 3.26-2.99 (m, 3H), | |
| | 2.78 (s, 3H), 2.24 (s, 3H), 1.17 (d, J = 6.4 Hz, 6H). | |
| 417 | ¹H NMR (400 MHz, methanol-d₄) δ 8.25 (s, 1H), 8.09 (s, 1H), | 552.1 |
| | 8.02 (s, 1H), 7.82-7.75 (m, 1H), 7.61 (s, 1H), 7.43 (t, J = 8.0 | |
| | Hz, 1H), 6.99 (d, J = 8.0 Hz, 1H), 6.07 (d, J = 44.4 Hz, 1H), | |
| | 5.10 (s, 2H), 3.99 (s, 2H), 3.69-3.44 (m, 2H), 3.25-3.02 (m, | |
| | 2H), 2.94 (s, 3H), 2.91-2.81 (m, 1H), 1.16 (d, J = 6.4 Hz, 6H). | |
| 418 | ¹H NMR (400 MHz, methanol-d₄) δ 8.25 (s, 1H), 8.08 (s, 1H), | 552.1 |
| | 8.01 (s, 1H), 7.78 (dd, J = 1.6, 8.0 Hz, 1H), 7.61 (s, 1H), 7.43 | |
| | (t, J = 8.0 Hz, 1H), 6.98 (d, J = 8.0 Hz, 1H), 6.07 (d, J = 44.4 | |
| | Hz, 1H), 5.10 (s, 2H), 3.97 (s, 2H), 3.70-3.43 (m, 2H), 3.25-3.02 | |
| | (m, 2H), 2.94 (s, 3H), 2.90-2.80 (m, 1H), 1.14 (d, J = 6.4 Hz, 6H). | |
| 419 | ¹H NMR (400 MHz, methanol-d₄) δ 8.26 (s 1H), 8.06 (s, 1H), | 566.2 |
| | 7.96 (s, 1H), 7.78 (d, J = 8.0 Hz, 1H), 7.60 (s, 1H), 7.43 (t, J = | |
| | 8.0 Hz, 1H), 6.99 (d, J = 8.0 Hz, 1H), 6.07 (d, J = 44.8 Hz, | |
| | 1H), 5.10 (s, 2H), 3.75 (s, 2H), 3.66-3.62 (m, 1H), 3.53- | |
| | 3.50 (m, 1H), 3.24-3.20 (m, 1H), 3.18-3.11 (m, 1H), 2.96- | |
| | 2.93 (m, 4H), 2.19 (s, 3H), 1.14 (d, J = 6.4 Hz, 6H). | |
| 420 | ¹H NMR (400 MHz, methanol-d₄) δ 8.26 (s 1H), 8.07 (s, 1H), | 566.3 |
| | 7.97 (s, 1H), 7.79 (d, J = 8.4 Hz, 1H), 7.61 (s, 1H), 7.43 (t, J = | |
| | 8.0 Hz, 1H), 6.98 (d, J = 8.0 Hz, 1H), 6.07 (d, J = 44.8 Hz, | |
| | 1H), 5.10 (s, 2H), 3.76 (s, 2H), 3.66-3.62 (m, 1H), 3.54- | |
| | 3.50 (m, 1H), 3.24-3.20 (m, 1H), 3.18-3.11 (m, 1H), 2.97- | |
| | 2.94 (m, 4H), 2.19 (s, 3H), 1.14 (d, J = 6.8 Hz, 6H). | |
| 421 | ¹H NMR (400 MHz, methanol-d₄) δ 8.18 (s, 1H), 8.06 (s, 1H), | 603.9 |
| | 7.97 (s, 1H), 7.75 (d, J = 8.4 Hz, 1H), 7.49 (s, 1H), 7.40 (t, J = | |
| | 8.0 Hz, 1H), 6.84 (d, J = 7.6 Hz, 1H), 5.09 (s, 2H), 3.75- | |
| | 3.71 (m, 2H), 3.37 (s, 2H), 3.31-3.29 (m, 2H), 3.07-3.02 | |
| | (m, 2H), 2.85-2.74 (m, 6H), 2.41-2.30 (m, 2H), 2.11 (s, | |
| | 3H), 2.10-2.00 (m, 2H), 1.80-1.70 (m, 1H), 1.40-1.35 (m, | |
| | 1H), 0.87 (t, J = 7.2 Hz, 3H). | |
| 422 | ¹H NMR (400 MHz, methanol-d₄) δ 8.89 (s, 1H), 8.21 (s, 1H), | 559.1 |
| | 7.74-7.63 (m, 3H), 7.61 (d, J = 2.0 Hz, 1H), 7.45-7.39 (m, | |
| | 2H), 6.91 (d, J = 8.0 Hz, 1H), 5.38 (s, 2H), 5.03 (s, 2H), 3.38- | |
| | 3.31 (m, 2H), 3.29-3.25 (m, 2H), 3.10-2.99 (m, 2H), 2.79 (s, 3H). | |
| 423 | ¹H NMR (400 MHz, methanol-d₄) δ 8.25 (s, 1H), 7.89 (s, 1H), | 555.4 |
| | 7.69 (d, J = 8.0 Hz, 1H), 7.52 (s, 1H), 7.35 (t, J = 8.0 Hz, 2H), | |
| | 6.84 (d, J = 7.6 Hz, 1H), 6.05 (d, J = 44.4 Hz, 1H), 3.80 (s, | |
| | 2H), 3.64-3.54 (m, 1H), 3.53-3.44 (m, 4H), 3.34-3.31 (m, | |
| | 1H), 3.20-3.08 (m, 4H), 2.90 (s, 3H), 2.24-2.18 (m, 1H), | |
| | 1.27 (s, 3H), 1.16-1.13 (m, 2H), 1.13-1.09 (m, 2H). | |
| 424 | ¹H NMR (400 MHz, methanol-d₄) δ 8.06-8.04 (m, 1H), 7.88- | 575.3 |
| | 7.83 (m, 2H), 7.57-7.51 (m, 2H), 7.35-7.30 (m, 1H), 6.72 | |
| | (d, J = 7.6 Hz, 1H), 4.93 (s, 2H), 3.72-3.63 (m, 2H), 3.42- | |
| | 3.24 (m, 6H), 3.04-2.81 (m, 5H), 2.76 (s, 3H), 2.56-2.52 | |
| | (m, 1H), 2.25-2.23 (m, 1H), 1.41-1.33 (m, 3H). | |
| 425 | ¹H NMR (400 MHz, methanol-d₄) δ 8.18 (s, 1H), 8.06 (s, 1H), | 575.3 |
| | 7.96 (s, 1H), 7.75 (d, J = 8.4 Hz, 1H), 7.48 (d, J = 2.0 Hz, 1H), | |
| | 7.40 (t, J = 8.0 Hz, 1H), 6.85 (d, J = 8.0 Hz, 1H), 5.09 (s, 2H), | |
| | 3.75 (s, 2H), 3.37 (s, 2H), 3.36-3.31 (m, 1H), 3.31-3.29 (m, 1H), | |
| | 3.06-3.02 (m, 2H), 2.94-2.86 (m, 3H), 2.80-2.76 (m, 5H), 2.17- | |
| | 2.11 (m, 1H), 1.85-1.79 (m, 1H), 1.04 (d, J = 6.4 Hz, 3H). | |
| 426 | ¹H NMR (400 MHz, methanol-d₄) δ 8.18 (s, 1H), 8.06 (s, 1H), | 589.3 |
| | 7.97 (s, 1H), 7.75 (d, J = 9.6 Hz, 1H), 7.48 (s, 1H), 7.40 (t, J = | |
| | 8.0 Hz, 1H), 6.85 (d, J = 8.0 Hz, 1H), 5.09 (s, 2H), 3.73- | |
| | 3.67 (m, 2H), 3.18 (s, 2H), 3.17-3.10 (m, 5H), 3.06-2.96 | |
| | (m, 2H), 2.89-2.87 (m, 1H), 2.78 (s, 3H), 2.77-2.75 (m, | |
| | 1H), 2.18-2.17 (m, 1H), 1.90-1.85 (m, 1H), 1.44-1.39 (m, | |
| | 2H), 0.93 (t, J = 8.0 Hz, 3H). | |
| 427 | ¹H NMR (400 MHz, methanol-d₄) δ 8.19 (s, 1H), 8.06 (s, 1H), | 589.3 |
| | 7.97 (s, 1H), 7.76-7.73 (m, 1H), 7.48 (s, 1H), 7.42-7.38 (m, | |
| | 1H), 6.85 (d, J = 7.6 Hz, 1H), 5.09 (s, 2H), 3.74-3.72 (m, | |
| | 2H), 3.37 (s, 2H), 3.32-3.31 (m, 1H), 3.31-3.29 (m, 1H), | |
| | 3.06-2.96 (m, 3H), 2.95-2.88 (m, 2H), 2.88-2.78 (m, 1H), | |
| | 2.78 (s, 3H), 2.75-2.65 (m, 1H), 2.19-2.17 (m, 1H), 1.88- | |
| | 1.84 (m, 1H), 1.44-1.39 (m, 2H), 0.93 (t, J = 7.6 Hz, 3H). | |
| 428 | ¹H NMR (400 MHz, methanol-d₄) δ 8.16 (s, 1H), 8.05 (s, 1H), | 603.1 |
| | 7.96 (s, 1H), 7.75-7.72 (m, 1H), 7.47 (s, 1H), 7.39 (t, J = 8.0 | |
| | Hz, 1H), 6.83 (d, J = 8.0 Hz, 1H), 5.08 (s, 2H), 3.77-3.70 (m, | |
| | 2H), 3.36 (s, 2H), 3.35-3.31 (m, 1H), 3.31-3.27 (m, 1H), | |
| | 3.05-2.83 (m, 5H), 2.77-2.73 (m, 4H), 2.48-2.40 (m, 1H), | |
| | 2.14-2.13 (m, 1H), 1.92-1.86 (m, 1H), 1.60-1.57 (m, 1H), | |
| | 0.95 (d, J = 6.8 Hz, 3H), 0.88 (d, J = 6.8 Hz, 3H). | |
| 429 | ¹H NMR (400 MHz, methanol-d₄) δ 8.17 (s, 1H), 8.06 (s, 1H), | 603.2 |
| | 7.96 (s, 1H), 7.74 (d, J = 8.4 Hz, 1H), 7.47 (s, 1H), 7.39 (t, J = | |
| | 8.0 Hz, 1H), 6.83 (d, J = 7.6 Hz, 1H), 5.08 (s, 2H), 3.77- | |
| | 3.71 (m, 2H), 3.36 (s, 2H), 3.35-3.31 (m, 1H), 3.31-3.29 | |
| | (m, 1H), 2.98-2.83 (m, 5H), 2.77-2.73 (m, 4H), 2.48-2.40 | |
| | (m, 1H), 2.20-2.13 (m, 1H), 1.92-1.86 (m, 1H), 1.60-1.58 | |
| | (m, 1H), 0.95 (d, J = 6.8 Hz, 3H), 0.88 (d, J = 6.8 Hz, 3H). | |
| 430 | ¹H NMR (400 MHz, methanol-d₄) δ 8.18 (s, 1H), 8.06 (s, 1H), | 601.4 |
| | 7.96 (s, 1H), 7.76-7.74 (m, 1H), 7.48 (s, 1H), 7.40 (t, J = 8.0 | |
| | Hz, 1H), 6.85 (d, J = 7.6 Hz, 1H), 5.09 (s, 2H), 3.74 (s, 2H), | |
| | 3.37 (s, 2H), 3.36-3.25 (m, 2H), 3.07-2.77 (m, 9H), 2.25-2.20 | |
| | (m, 1H), 2.10-2.03 (m, 1H), 1.96-1.93 (m, 1H), 0.75-0.74 (m, | |
| | 1H), 0.50-0.45 (m, 2H), 0.30-0.20 (m, 1H), 0.16-0.13 (m, 1H). | |
| 431 | ¹H NMR (400 MHz, methanol-d₄) δ 8.18 (s, 1H), 8.08 (s, 1H), | 601.4 |
| | 7.97 (s, 1H), 7.74 (d, J = 8.0 Hz, 1H), 7.46 (s, 1H), 7.41 (t, J = | |
| | 8.0 Hz, 1H), 6.88 (d, J = 7.6 Hz, 1H), 5.10 (s, 2H), 3.84- | |
| | 3.75 (m, 2H), 3.37 (s, 2H), 3.37-3.31 (m, 2H), 3.26-2.88 | |
| | (m, 6H), 2.78 (s, 3H), 2.45-2.20 (m, 3H), 1.00-0.90 (m, | |
| | 1H), 0.70-0.50 (m, 2H), 0.50-0.25 (m, 2H). | |
| 432 | ¹H NMR (400 MHz, methanol-d₄) δ 8.33 (s, 1H), 8.08 (s, 1H), | 653.3 |
| | 7.94 (s, 1H), 7.81-7.78 (m, 1H), 7.65 (s, 1H), 7.47 (t, J = 8.0 | |
| | Hz, 1H), 7.06 (d, J = 8.0 Hz, 1H), 5.09 (s, 2H), 4.34 (d, J = | |
| | 14.0 Hz, 1H), 3.77-3.67 (m, 2H), 3.28-3.21 (m, 2H), 2.96 | |
| | (s, 3H), 2.87 (d, J = 11.2 Hz, 1H), 2.74 (d, J = 9.6 Hz, 2H), | |
| | 2.39-2.34 (m, 6H), 2.33-2.17 (m, 3H), 1.02 (d, J = 6.4 Hz, | |
| | 3H), 0.98 (d, J = 6.8 Hz, 3H). | |
| 433 | ¹H NMR (400 MHz, methanol-d₄) δ 8.17 (s, 1H), 8.11 (s, 1H), | 615.3 |
| | 7.98 (s, 1H), 7.74 (d, J = 8.0 Hz, 1H), 7.47 (s, 1H), 7.38 (t, J = | |
| | 8.0 Hz, 1H), 6.85 (d, J = 7.6 Hz, 1H), 5.08 (s, 2H), 3.37 (s, | |
| | 2H), 3.26-2.98 (m, 7H), 2.78-2.68 (m, 5H), 2.37-2.24 (m, | |
| | 6H), 1.65-1.60 (m, 1H), 0.73-0.69 (m, 2H), 0.60-0.56 (m, | |
| | 1H), 0.42-0.40 (m, 1H), 0.21-0.16 (m, 1H). | |
| 434 | ¹H NMR (400 MHz, methanol-d₄) δ 8.18 (s, 1H), 8.08 (s, 1H), | 603.4 |
| | 7.95 (s, 1H), 7.75 (d, J = 8.0 Hz, 1H), 7.48 (s, 1H), 7.40 (t, J = | |
| | 8.0 Hz, 1H), 6.85 (d, J = 8.0 Hz, 1H), 5.09 (s, 2H), 4.27 (d, | |
| | J = 14.4 Hz, 1H), 3.43-3.31 (m, 4H), 3.07-3.02 (m, 3H), | |
| | 2.80-2.77 (m, 1H), 2.72 (s, 3H), 2.69-2.65 (m, 2H), 2.44- | |
| | 2.40 (m, 1H), 2.32-2.30 (m, 1H), 2.29 (s, 3H), 2.21-2.10 | |
| | (m, 2H), 1.8-1.70 (m, 1H), 1.62-1.58 (m, 1H), 0.98 (t, J = | |
| | 7.6 Hz, 3H). | |
| 435 | ¹H NMR (400 MHz, methanol-d₄) δ 8.18 (s, 1H), 8.14 (s, 1H), | 532.2 |
| | 8.06 (s, 1H), 7.75 (d, J = 8.0 Hz, 1H), 7.49 (s, 1H), 7.43-7.38 | |
| | (m, 1H), 6.85 (d, J = 8.0 Hz, 1H), 5.09 (s, 2H), 3.39-3.31 (m, | |
| | 4H), 3.31-3.25 (m, 1H), 3.11-2.99 (m, 2H), 2.76 (s, 3H), | |
| | 1.19-1.15 (m, 1H), 0.73-0.67 (m, 1H), 0.54-0.50 (m, 2H), | |
| | 0.37-0.35 (m, 1H). | |
| 436 | ¹H NMR (400 MHz, methanol-d₄) δ 8.18 (s, 1H), 8.12 (s, 1H), | 601.2 |
| | 7.99 (s, 1H), 7.74 (d, J = 6.4 Hz, 1H), 7.47 (s, 1H), 7.41 (t, J = | |
| | 8.0 Hz, 1H), 6.87 (d, J = 7.6 Hz, 1H), 5.09 (s, 2H), 4.82- | |
| | 4.77 (m, 1H), 3.49-3.31 (m, 4H), 3.16-2.94 (m, 5H), 2.89- | |
| | 2.85 (m, 2H), 2.78 (s, 3H), 2.73-2.70 (m, 1H), 2.24-2.18 | |
| | (m, 1H), 1.61-1.58 (m, 1H), 0.76-0.71 (m, 2H), 0.60-0.57 | |
| | (m, 1H), 0.44-0.41 (m, 1H), 0.20-0.18 (m, 1H). | |
| 437 | ¹H NMR (400 MHz, methanol-d₄) δ 8.17 (s, 1H), 8.06 (s, 1H), | |
| | 7.97 (s, 1H), 7.75 (dd, J = 1.6, 8.0 Hz, 1H), 7.48 (s, 1H), 7.40 | |
| | (t, J = 8.0 Hz, 1H), 6.85 (d, J = 8.0 Hz, 1H), 5.08 (s, 2H), 3.72 | |
| | (s, 2H), 3.37 (s, 2H), 3.35-3.22 (m, 2H), 3.11-2.96 (m, 2H), | |
| | 2.91-2.82 (m, 4H), 2.78 (s, 3H), 2.50 (br s, 4H). | |
| 438 | ¹H NMR (400 MHz, methanol-d₄) δ 8.16 (s, 1H), 8.05 (s, 1H), | 617.2 |
| | 7.96 (s, 1H), 7.74 (d, J = 7.6 Hz, 1H), 7.47 (s, 1H), 7.39 (t, J = | |
| | 8.0 Hz, 1H), 6.83 (d, J = 8.0 Hz, 1H), 5.08 (s, 2H), 3.78- | |
| | 3.66 (m, 2H), 3.36 (s, 3H), 3.30-3.24 (m, 1H), 3.09-3.01 | |
| | (m, 2H), 2.82-2.73 (m, 6H), 2.40-2.29 (m, 2H), 2.27-2.20 | |
| | (m, 3H), 2.20-2.14 (m, 1H), 2.04-2.01 (m, 2H), 0.88 (d, J = | |
| | 6.8 Hz, 6H). | |
| 439 | ¹H NMR (400 MHz, methanol-d₄) δ 8.18 (s, 1H), 8.06 (s, 1H), | 617.3 |
| | 7.97 (s, 1H), 7.76-7.73 (m, 1H), 7.49 (s, 1H), 7.40 (t, J = 8.0 | |
| | Hz, 1H), 6.84 (d, J = 7.6 Hz, 1H), 5.09 (s, 2H), 3.79-3.67 (m, | |
| | 2H), 3.37 (s, 2H), 3.28-3.25 (m, 1H), 3.07-3.02 (m, 2H), | |
| | 2.83-2.74 (m, 7H), 2.31-2.30 (m, 1H), 2.30-2.20 (m, 4H), | |
| | 2.20-2.10 (m, 1H), 2.06-2.00 (m, 2H), 0.90-0.88 (m, 6H). | |
| 440 | ¹H NMR (400 MHz, methanol-d₄) δ 8.18 (s, 1H), 8.10 (s, 1H), | 590.1 |
| | 8.03 (s, 1H), 7.75 (d, J = 8.0 Hz, 1H), 7.48 (s, 1H), 7.40 (t, J = | |
| | 8.0 Hz, 1H), 6.85 (d, J = 7.6 Hz, 1H), 5.10 (s, 2H), 3.80 (br | |
| | s, 1H), 3.37 (s, 2H), 3.31-3.23 (m, 2H), 3.12-2.97 (m, 2H), | |
| | 2.78 (s, 3H), 2.69-2.56 (m, 1H), 2.51-2.21 (m, 3H), 1.95- | |
| | 1.76 (m, 2H), 1.69-1.41 (m, 3H), 1.20 (s, 3H). | |
| 441 | ¹H NMR (400 MHz, methanol-d₄) δ 8.20 (s, 1H), 8.10 (s, 1H), | 590.1 |
| | 8.03 (s, 1H), 7.77 (d, J = 8.0 Hz, 1H), 7.51 (s, 1H), 7.42 (t, J = | |
| | 8.0 Hz, 1H), 6.86 (d, J = 7.6 Hz, 1H), 5.11 (s, 2H), 3.74 (s, | |
| | 2H), 3.39 (s, 2H), 3.35-3.24 (m, 2H), 3.12-2.97 (m, 2H), | |
| | 2.80 (s, 3H), 2.63-2.51 (m, 1H), 2.43-2.20 (m, 3H), 1.91- | |
| | 1.77 (m, 1H), 1.66-1.43 (m, 3H), 1.22 (s, 3H). | |
| 442 | ¹H NMR (400 MHz, methanol-d₄) δ 8.45 (br s, 1H), 8.17 (s, | 546.3 |
| | 1H), 7.94 (s, 1H), 7.88 (d, J = 6.0 Hz, 1H), 7.73 (d, J = 8.0 Hz, | |
| | 1H), 7.45-7.39 (m, 2H), 6.90 (d, J = 6.8 Hz, 1H), 5.08 (s, | |
| | 2H), 4.38-4.34 (m, 1H), 4.20-3.97 (m, 3H), 3.37 (s, 3H), 3.29- | |
| | 3.21 (m, 5H), 3.06-3.00 (m, 2H), 2.92 (s, 2H), 2.78 (s, 3H). | |
| 443 | ¹H NMR (400 MHz, methanol-d₄) δ 8.17 (s, 1H), 8.06 (s, 1H), | 590.2 |
| | 7.98 (s, 1H), 7.75 (d, J = 8.0 Hz, 1H), 7.49 (s, 1H), 7.40 (t, J = | |
| | 8.0 Hz, 1H), 6.84 (d, J = 8.0 Hz, 1H), 5.09 (s, 2H), 3.73 (d, | |
| | J = 3.2 Hz, 2H), 3.74-3.32 (m, 4H), 3.31-3.25 (m, 4H), 3.10- | |
| | 2.95 (m, 2H), 2.85-2.80 (m, 1H), 2.78 (s, 3H), 2.64-2.61 | |
| | (m, 1H), 2.25-2.10 (m, 2H), 1.94-1.90 (m, 1H), 1.78-1.75 | |
| | (m, 1H), 1.58-1.54 (m, 1H), 1.33-1.30 (m, 1H). | |
| 444 | ¹H NMR (400 MHz, methanol-d₄) δ 8.17 (s, 1H), 8.00 (s, 1H), | 589.2 |
| | 7.92 (s, 1H), 7.76-7.73 (m, 1H), 7.48 (d, J = 2.0 Hz, 1H), | |
| | 7.42 (t, J = 8.0 Hz, 1H), 6.85 (d, J = 7.6 Hz, 1H), 5.10 (s, 2H), | |
| | 4.60 (s, 2H), 3.42 (d, J = 5.6 Hz, 2H), 3.40 (s, 2H), 3.37-3.31 | |
| | (m, 2H), 3.31-3.22 (m, 2H), 3.06-3.02 (m, 2H), 2.78 (s, | |
| | 3H), 2.76-2.73 (m, 2H), 2.37 (s, 3H). | |
| 445 | ¹H NMR (400 MHz, methanol-d₄) δ 8.25 (s, 1H), 8.07 (s, 1H), | 552.2 |
| | 8.00 (s, 1H), 7.80-7.76 (m, 1H), 7.60 (s, 1H), 7.43 (t, J = 8.0 | |
| | Hz, 1H), 6.98 (d, J = 8.0 Hz, 1H), 6.06 (d, J = 44.4 Hz, 1H), | |
| | 5.10 (s, 2H), 3.97 (s, 2H), 3.69-3.61 (m, 1H), 3.53-3.46 (m, | |
| | 1H), 3.21-3.07 (m, 2H), 2.94 (s, 3H), 2.61-2.57 (m, 2H), | |
| | 1.63-1.53 (m, 2H), 0.95 (t, J = 7.2 Hz, 3H). | |
| 446 | ¹H NMR (400 MHz, methanol-d₄) δ 8.25 (s, 1H), 8.07 (s, 1H), | 552.2 |
| | 8.00 (s, 1H), 7.80-7.77 (m, 1H), 7.60 (s, 1H), 7.43 (t, J = 8.0 | |
| | Hz, 1H), 6.98 (d, J = 8.0 Hz, 1H), 6.06 (d, J = 44.8 Hz, 1H), | |
| | 5.10 (s, 2H), 3.97 (s, 2H), 3.69-3.62 (m, 1H), 3.53-3.46 (m, | |
| | 1H), 3.21-3.08 (m, 2H), 2.94 (s, 3H), 2.61-2.57 (m, 2H), | |
| | 1.63-1.53 (m, 2H), 0.95 (t, J = 7.6 Hz, 3H). | |
| 447 | ¹H NMR (400 MHz, methanol-d₄) δ 8.26 (s, 1H), 8.08 (s, 1H), | 570.3 |
| | 8.00 (s, 1H), 7.79 (d, J = 8.0 Hz, 1H), 7.61 (s, 1H), 7.43 (t, J = | |
| | 8.0 Hz, 1H), 6.98 (d, J = 8.0 Hz, 1H), 6.07 (d, J = 44.4 Hz, | |
| | 1H), 5.10 (s, 2H), 4.59-4.56 (m, 1H), 4.47-4.44 (m, 1H), | |
| | 3.97 (s, 2H), 3.63-3.49 (m, 2H), 3.21-3.11 (m, 2H), 2.94 | |
| | (s, 3H), 2.74 (t, J = 7.2 Hz, 2H), 1.98-1.87 (m, 2H). | |
| 448 | ¹H NMR (400 MHz, methanol-d₄) δ 8.26 (s, 1H), 8.08 (s, 1H), | 570.2 |
| | 8.00 (s, 1H), 7.79 (d, J = 6.0 Hz, 1H), 7.61 (s, 1H), 7.43 (t, J = | |
| | 8.0 Hz, 1H), 6.98 (d, J = 8.0 Hz, 1H), 6.07 (d, J = 44.4 Hz, | |
| | 1H), 5.10 (s, 2H), 4.59-4.56 (m, 1H), 4.47-4.44 (m, 1H), | |
| | 3.97 (s, 2H), 3.63-3.50 (m, 2H), 3.21-3.11 (m, 2H), 2.94 | |
| | (s, 3H), 2.75 (t, J = 7.2 Hz, 2H), 1.98-1.88 (m, 2H). | |
| 449 | ¹H NMR (400 MHz, methanol-d₄) δ 8.25 (s, 1H), 8.08 (s, 1H), | 564.2 |
| | 8.01 (s, 1H), 7.78 (d, J = 8.4 Hz, 1H), 7.61 (s, 1H), 7.43 (t, J = | |
| | 8.0 Hz, 1H), 6.98 (d, J = 8.0 Hz, 1H), 6.07 (d, J = 44.8 Hz, | |
| | 1H), 5.10 (s, 2H), 4.00 (s, 2H), 3.66-3.48 (m, 2H), 3.21- | |
| | 3.08 (m, 2H), 2.94 (s, 3H), 2.50 (d, J = 6.8 Hz, 2H), 1.02- | |
| | 1.00 (m, 1H), 0.56-0.50 (m, 2H), 0.19-0.14 (m, 2H). | |
| 450 | ¹H NMR (400 MHz, methanol-d₄) δ 8.26 (s, 1H), 8.08 (s, 1H), | 564.2 |
| | 8.01 (s, 1H), 7.78 (d, J = 6.4 Hz, 1H), 7.61 (s, 1H), 7.43 (t, J = | |
| | 8.0 Hz, 1H), 6.99 (d, J = 8.0 Hz, 1H), 6.07 (d, J = 44.4 Hz, | |
| | 1H), 5.10 (s, 2H), 4.00 (s, 2H), 3.66-3.50 (m, 2H), 3.21- | |
| | 3.11 (m, 2H), 2.94 (s, 3H), 2.50 (d, J = 6.8 Hz, 2H), 1.02- | |
| | 1.00 (m, 1H), 0.56-0.51 (m, 2H), 0.19-0.16 (m, 2H). | |
| 451 | ¹H NMR (400 MHz, methanol-d₄) δ 8.26 (s, 1H), 8.08 (s, 1H), | 566.1 |
| | 8.01 (s, 1H), 7.78 (d, J = 8.4 Hz, 1H), 7.61 (s, 1H), 7.43 (t, J = | |
| | 8.0 Hz, 1H), 6.99 (d, J = 7.6 Hz, 1H), 6.07 (d, J = 44.4 Hz, | |
| | 1H), 5.10 (s, 2H), 3.96 (s, 2H), 3.66-3.49 (m, 2H), 3.21- | |
| | 3.08 (m, 2H), 2.94 (s, 3H), 2.42 (d, J = 6.4 Hz, 2H), 1.85- | |
| | 1.78 (m, 1H), 0.94 (d, J = 6.4 Hz, 6H). | |
| 452 | ¹H NMR (400 MHz, methanol-d₄) δ 8.25 (s, 1H), 8.11 (s, 1H), | 566.1 |
| | 8.04 (s, 1H), 7.78 (d, J = 8.0 Hz, 1H), 7.61 (s, 1H), 7.44 (t, J = | |
| | 8.0 Hz, 1H), 7.00 (d, J = 7.6 Hz, 1H), 6.07 (d, J = 44.4 Hz, | |
| | 1H), 5.11 (s, 2H), 4.06 (s, 2H), 3.66-3.46 (m, 2H), 3.21- | |
| | 3.08 (m, 2H), 2.94 (s, 3H), 2.53 (d, J = 6.8 Hz, 2H), 1.91- | |
| | 1.83 (m, 1H), 0.97 (d, J = 6.8 Hz, 6H). | |
| 453 | ¹H NMR (400 MHz, methanol-d₄) δ 8.18 (s, 1H), 8.14 (s, 1H), | 532.2 |
| | 8.06 (s, 1H), 7.77-7.74 (m, 1H), 7.49 (s, 1H), 7.40 (t, J = 8.0 | |
| | Hz, 1H), 6.85 (d, J = 8.0 Hz, 1H), 5.10 (s, 2H), 3.40-3.37 (m, | |
| | 4H), 3.31-3.25 (m, 1H), 3.11-3.03 (m, 2H), 2.78 (s, 3H), | |
| | 1.20-1.16 (m, 1H), 0.73-0.70 (m, 1H), 0.57-0.50 (m, 2H), | |
| | 0.37-0.35 (m, 1H). | |
| 454 | ¹H NMR (400 MHz, methanol-d₄) δ 8.50 (s, 1H), 8.09 (s, 1H), | 669.3 |
| | 7.94 (s, 1H), 7.77 (d, J = 8.0 Hz, 1H), 7.62 (s, 1H), 7.41 (t, J = | |
| | 8.0 Hz, 1H), 6.95 (d, J = 7.6 Hz, 1H), 6.25 (d, J = 45.6 Hz, | |
| | 1H), 5.61 (d, J = 6.4 Hz, 1H), 5.36 (d, J = 5.6 Hz, 1H), 5.25- | |
| | 5.22 (m, 1H), 5.08 (s, 2H), 5.02-5.00 (m, 1H), 4.34 (d, J = | |
| | 14.0 Hz, 1H), 3.57-3.54 (m, 1H), 3.35-3.31 (m, 1H), 2.78- | |
| | 2.71 (m, 3H), 2.37-2.28 (m, 6H), 2.15-2.07 (m, 2H), 1.79- | |
| | 1.75 (m, 2H), 1.75-1.40 (m, 3H), 1.40-1.15 (m, 5H), 1.01 | |
| | (d, J = 6.4 Hz, 3H), 0.98 (d, J = 6.8 Hz, 3H). | |
| 455 | ¹H NMR (400 MHz, methanol-d₄) δ 8.51 (s, 1H), 8.09 (s, 1H), | 669.4 |
| | 7.95 (s, 1H), 7.77 (d, J = 8.0 Hz, 1H), 7.63 (s, 1H), 7.42 (t, J = | |
| | 8.0 Hz, 1H), 6.95 (d, J = 8.0 Hz, 1H), 6.25 (d, J = 45.6 Hz, | |
| | 1H), 5.62 (d, J = 6.8 Hz, 1H), 5.37 (d, J = 6.0 Hz, 1H), 5.26- | |
| | 5.24 (m, 1H), 5.09 (s, 2H), 5.03-5.00 (m, 1H), 4.33 (d, J = | |
| | 14.4 Hz, 1H), 3.58-3.54 (m, 1H), 3.35-3.31 (m, 1H), 2.81- | |
| | 2.71 (m, 3H), 2.37-2.28 (m, 6H), 2.15-2.07 (m, 2H), 1.79- | |
| | 1.75 (m, 2H), 1.75-1.40 (m, 3H), 1.40-1.15 (m, 5H), 1.01 | |
| | (d, J = 6.4 Hz, 3H), 0.98 (d, J = 6.4 Hz, 3H). | |
| 456 | ¹H NMR (400 MHz, methanol-d₄) δ 8.18 (s, 1H), 8.05 (s, 1H), | 555.3 |
| | 7.98 (s, 1H), 6.99 (s, 1H), 6.53 (s, 1H), 5.96 (s, 1H), 5.00 (s, | |
| | 2H), 3.97 (s, 2H), 3.55-3.51 (m, 1H), 3.28 (s, 2H), 2.85 (s, | |
| | 3H), 2.62-2.51 (m, 4H), 2.44-2.40 (m, 2H), 2.26-2.20 (m, | |
| | 1H), 2.00-1.85 (m, 1H), 1.62-1.55 (m, 2H), 1.15 (d, J = 6.4 | |
| | Hz, 3H), 1.57 (d, J = 7.6 Hz, 3H), 0.95 (t, J = 7.2 Hz, 3H). | |
| 457 | ¹H NMR (400 MHz, methanol-d₄) δ 8.18-8.16 (m, 2H), 8.10 | 553.1 |
| | (s, 1H), 6.97 (s, 1H), 6.54 (s, 1H), 6.00 (s, 1H), 5.06 (s, 2H), | |
| | 4.59 (s, 2H), 4.35 (s, 2H), 3.08-3.02 (m, 2H), 2.96-2.89 (m, | |
| | 2H), 2.86 (s, 3H), 2.59-2.50 (m, 2H), 2.48-2.38 (m, 2H), | |
| | 2.35-2.19 (m, 1H), 1.98-1.87 (m, 1H), 1.20 (t, J = 7.2 Hz, | |
| | 3H), 0.94-0.82 (m, 1H), 0.75-0.66 (m, 2H), 0.43-0.33 (m, 2H). | |
| 458 | ¹H NMR (400 MHz, methanol-d₄) δ 8.21-8.19 (m, 2H), 8.13 | 541.1 |
| | (s, 1H), 6.98 (s, 1H), 6.54 (s, 1H), 6.02 (s, 1H), 5.08 (s, 2H), | |
| | 4.44 (s, 2H), 3.11-3.03 (m, 4H), 2.87 (s, 3H), 2.54-2.05 (m, | |
| | 2H), 2.44-2.39 (m, 2H), 2.32-2.22 (m, 1H), 1.99-1.87 (m, | |
| | 1H), 1.83-1.71 (m, 2H), 1.40-1.28 (m, 1H), 1.20 (t, J = 7.2 | |
| | Hz, 3H), 1.06 (t, J = 7.2 Hz, 3H), 0.93-0.80 (m, 1H). | |
| 459 | ¹H NMR (400 MHz, DMSO-d₆) δ 8.14 (s, 1H), 7.97 (d, J = 8.0 | 567.2 |
| | Hz, 2H), 7.01 (s, 1H), 6.48 (s, 1H), 5.83 (s, 1H), 5.44 (d, J = | |
| | 8.0 Hz, 1H), 4.96 (s, 2H), 3.90 (s, 2H), 3.13 (s, 2H), 2.78 (s, | |
| | 3H), 2.46-2.42 (m, 2H), 2.37-2.21 (m, 6H), 2.20-2.06 (m, | |
| | 1H), 1.87-1.74 (m, 1H), 1.06 (d, J = 6.4 Hz, 6H), 0.93-0.85 | |
| | (m, 1H), 0.42-0.37 (m, 2H), 0.09-0.05 (m, 2H). | |
| 460 | ¹H NMR (400 MHz, methanol-d₄) δ 8.26 (s, 1H), 8.09 (s, 1H), | 649.3 |
| | 7.95 (s, 1H), 7.78 (d, J = 8.4 Hz, 1H), 7.59 (s, 1H), 7.44 (t, J = | |
| | 8.0 Hz, 1H), 6.99 (d, J = 7.6 Hz, 1H), 6.07 (d, J = 44.4 Hz, | |
| | 1H), 5.14 (s, 2H), 4.34 (d, J = 14.4 Hz, 1H), 3.66-3.62 (m, | |
| | 2H), 3.50-3.34 (m, 1H), 3.25-3.00 (m, 2H), 2.94 (s, 3H), | |
| | 2.90-2.87 (m, 1H), 2.76-2.73 (m, 2H), 2.55-2.30 (m, 5H), | |
| | 2.09-2.06 (m, 2H), 1.11 (t, J = 6.8 Hz, 3H), 1.02 (d, J = 6.4 | |
| | Hz, 3H), 0.98 (d, J = 6.4 Hz, 3H). | |
| 461 | ¹H NMR (400 MHz, methanol-d₄) δ 8.25 (s, 1H), 8.09 (s, 1H), | 649.3 |
| | 7.94 (s, 1H), 7.77 (d, J = 6.8 Hz, 1H), 7.59 (s, 1H), 7.43 (t, J = | |
| | 8.0 Hz, 1H), 6.99 (d, J = 8.0 Hz, 1H), 6.06 (d, J = 44.4 Hz, | |
| | 1H), 5.13 (s, 2H), 4.33 (d, J = 14.4 Hz, 1H), 3.66-3.49 (m, | |
| | 2H), 3.35-3.31 (m, 1H), 3.25-3.00 (m, 2H), 2.95 (s, 3H), | |
| | 2.90-2.85 (m, 1H), 2.80-2.70 (m, 2H), 2.48-2.30 (m, 5H), | |
| | 2.09-2.05 (m, 2H), 1.11 (t, J = 7.2 Hz, 3H), 1.06 (d, J = 6.8 | |
| | Hz, 3H), 0.98 (d, J = 6.8 Hz, 3H). | |
| 462 | ¹H NMR (400 MHz, DMSO-d₆) δ 8.14 (s, 1H), 8.00 (s, 1H), | 581.2 |
| | 7.98 (s, 1H), 7.02 (s, 1H), 6.74 (s, 1H), 5.83 (s, 1H), 5.44 (d, | |
| | J = 8.0 Hz, 1H), 4.95 (s, 2H), 3.81 (s, 2H), 3.13 (s, 2H), 2.78 | |
| | (s, 3H), 2.47-2.41 (m, 2H), 2.33-2.23 (m, 3H), 2.17-2.07 | |
| | (m, 1H), 2.01-1.94 (m, 2H), 1.86-1.77 (m, 1H), 1.76-1.59 | |
| | (m, 5H), 1.22 (s, 3H), 1.06 (d, J = 6.4 Hz, 6H). | |
| 463 | ¹H NMR (400 MHz, methanol-d₄) δ 8.18 (s, 1H), 8.06 (s, 1H), | 567.2 |
| | 7.99 (s, 1H), 6.98 (s, 1H), 6.56 (s, 1H), 5.94 (s, 1H), 5.00 (s, | |
| | 2H), 3.86 (s, 2H), 3.28 (s, 2H), 3.07-3.00 (m, 2H), 2.84 (s, | |
| | 3H), 2.54-2.50 (m, 2H), 2.50-2.45 (m, 2H), 2.40-2.20 (m, | |
| | 1H), 2.12-2.08 (m, 2H), 1.90-1.81 (m, 3H), 1.80-1.78 (m, | |
| | 2H), 1.38 (s, 3H), 1.19 (t, J = 7.2 Hz, 3H). | |
| 464 | ¹H NMR (400 MHz, methanol-d₄) δ 8.25 (s, 1H), 8.10 (s, 1H), | 578.0 |
| | 8.02 (s, 1H), 7.78 (d, J = 7.2 Hz, 1H), 7.61 (s, 1H), 7.43 (t, J = | |
| | 8.0 Hz, 1H), 6.98 (d, J = 8.0 Hz, 1H), 6.68 (d, J = 44.4 Hz, | |
| | 1H), 5.10 (s, 2H), 3.91 (s, 2H), 3.66-3.50 (m, 2H), 3.30- | |
| | 3.14 (m, 2H), 2.94 (s, 3H), 2.14-2.11 (m, 2H), 1.93-1.79 | |
| | (m, 4H), 1.41 (s, 3H). | |
| 465 | ¹H NMR (400 MHz, methanol-d₄) δ 8.24 (s, 1H), 8.13 (s, 1H), | 578.1 |
| | 8.06 (s, 1H), 7.77 (d, J = 8.4 Hz, 1H), 7.60 (s, 1H), 7.43 (t, J = | |
| | 8.0 Hz, 1H), 7.00 (d, J = 8.4 Hz, 1H), 6.06 (d, J = 44.4 Hz, | |
| | 1H), 5.12 (s, 2H), 4.03 (s, 2H), 3.62-3.48 (m, 2H), 3.21- | |
| | 3.07 (m, 2H), 2.94 (s, 3H), 2.23-2.20 (m, 2H), 1.97-1.85 | |
| | (m, 4H), 1.48 (s, 3H). | |
| 466 | ¹H NMR (400 MHz, methanol-d₄) δ 8.19 (s, 1H), 8.06 (s, 1H), | 603.2 |
| | 7.99 (s, 1H), 7.02 (s, 1H), 6.63 (s, 1H), 5.94 (s, 1H), 5.01 (s, | |
| | 2H), 3.88 (s, 2H), 3.40-3.31 (m, 2H), 3.29-3.20 (m, 2H), | |
| | 3.06-2.98 (m, 4H), 2.86 (s, 3H), 2.12-2.09 (m, 2H), 1.90- | |
| | 1.79 (m, 4H), 1.38 (s, 3H), 1.18 (t, J = 7.2 Hz, 3H). | |
| 467 | ¹H NMR (400 MHz, methanol-d₄) δ 8.17 (s, 1H), 8.09 (s, 1H), | 549.1 |
| | 8.01 (s, 1H), 7.73 (d, J = 8.0 Hz, 1H), 7.55 (s, 1H), 7.40 (t, J = | |
| | 8.0 Hz, 1H), 6.90 (d, J = 8.0 Hz, 1H), 5.08 (s, 2H), 3.87 (s, | |
| | 2H), 3.43 (s, 2H), 3.19-3.16 (m, 1H), 3.03-2.99 (m, 4H), 2.83 | |
| | (s, 3H), 2.12-2.08 (m, 2H), 1.90-1.78 (m, 4H), 1.38 (s, 3H). | |
| 468 | ¹H NMR (400 MHz, methanol-d₄) δ 8.18 (s, 1H), 8.09 (s, 1H), | 549.3 |
| | 8.01 (s, 1H), 7.74-7.71 (m, 1H), 7.39 (t, J = 8.0 Hz, 2H), 6.74 | |
| | (d, J = 8.4 Hz, 1H), 5.08 (s, 2H), 3.87 (s, 2H), 3.67-3.61 (m, | |
| | 1H), 3.33 (s, 2H), 3.14-3.08 (m, 2H), 2.79-2.75 (m, 1H), | |
| | 2.75 (s, 3H), 2.75-2.72 (m, 1H), 2.12-2.08 (m, 2H), 1.90- | |
| | 1.80 (m, 4H), 1.38 (s, 3H). | |
| 469 | ¹H NMR (400 MHz, DMSO-d₆) δ 8.17 (s, 1H), 8.08 (s, 1H), | 560.1 |
| | 8.03 (s, 1H), 7.74 (d, J = 8.0 Hz, 1H), 7.49 (s, 1H), 7.40 (t, J = | |
| | 8.0 Hz, 1H), 6.84 (d, J = 7.6 Hz, 1H), 5.08 (s, 2H), 4.51- | |
| | 4.46 (m, 1H), 3.37 (s, 2H), 3.28-3.26 (m, 3H), 3.10-3.02 | |
| | (m, 2H), 2.78 (s, 3H), 2.41-2.38 (m, 1H), 2.25-2.10 (m, | |
| | 1H), 1.82-1.75 (m, 1H), 1.68-1.65 (m, 1H), 1.65-1.50 (m, | |
| | 2H), 1.04-0.99 (m, 3H). | |
| 470 | ¹H NMR (400 MHz, DMSO-d₆) δ 8.35 (s, 1H), 7.99-7.96 (m, | 511.8 |
| | 1H), 7.74 (s, 2H), 7.51 (s, 1H), 7.36 (t, J = 8.0 Hz, 1H), 6.97- | |
| | 6.94 (m, 1H), 6.26 (d, J = 46.0 Hz, 1H), 5.36 (d, J = 6.8 Hz, | |
| | 1H), 5.21 (d, J = 6.0 Hz, 1H), 5.13 (d, J = 6.8 Hz, 1H), 4.94- | |
| | 4.86 (m, 2H), 4.84-4.82 (m, 1H), 3.74 (s, 2H), 3.19 (s, 3H), | |
| | 1.98-1.94 (m, 2H), 1.73-1.61 (m, 4H), 1.22 (s, 3H). | |
| 471 | ¹H NMR (400 MHz, methanol-d₄) δ 8.29 (s, 1H), 7.89 (s, 1H), | 556.2 |
| | 7.83-7.81 (m, 2H), 7.49 (s, 1H), 7.43 (t, J = 8.0 Hz, 1H), 6.93- | |
| | 6.90 (m, 1H), 6.59 (d, J = 45.2 Hz, 1H), 5.49 (d, J = 6.4 Hz, | |
| | 1H), 5.32 (d, J = 6.4 Hz, 1H), 5.21 (d, J = 6.4 Hz, 1H), 5.04- | |
| | 5.01 (m, 1H), 4.89-4.84 (m, 2H), 3.80 (s, 2H), 3.11 (s, 3H), | |
| | 2.14-2.08 (m, 2H), 1.90-1.78 (m, 4H), 1.38 (s, 3H). | |
| 472 | ¹H NMR (400 MHz, methanol-d₄) δ 8.21 (s, 1H), 8.08 (s, 1H), | 566.2 |
| | 8.01 (s, 1H), 7.55 (s, 1H), 7.12 (s, 1H), 6.29 (s, 1H), 5.08- | |
| | 5.03 (m, 6H), 3.87 (s, 2H), 3.63 (s, 2H), 2.87 (s, 3H), 2.12- | |
| | 2.08 (m, 2H), 1.91-1.79 (m, 5H), 1.38 (s, 3H), 0.99-0.95 | |
| | (m, 2H), 0.64-0.61 (m, 2H). | |
| 473 | ¹H NMR (400 MHz, methanol-d₄) δ 8.22 (s, 1H), 8.08 (s, 1H), | 570.3 |
| | 8.01 (s, 1H), 7.47 (t, J = 2.0 Hz, 1H), 6.86 (s, 1H), 6.23 (s, | |
| | 1H), 5.07-5.03 (m, 6H), 4.01-3.95 (m, 2H), 3.87 (s, 2H), | |
| | 3.65 (s, 2H), 2.96 (s, 3H), 2.12-2.09 (m, 2H), 1.91-1.78 (m, | |
| | 4H), 1.37 (t, J = 7.2 Hz, 6H). | |
| 474 | ¹H NMR (400 MHz, methanol-d₄) δ 8.21 (s, 1H), 8.07 (s, 1H), | 569.4 |
| | 8.00 (s, 1H), 7.06 (d, J = 2.0 Hz, 1H), 6.50 (d, J = 1.6 Hz, 1H), | |
| | 5.81 (d, J = 1.6 Hz, 1H), 5.02 (s, 6H), 2.86 (s, 2H), 3.62 (s, | |
| | 2H), 3.07-3.01 (m, 2H), 2.94 (s, 3H), 2.14-2.08 (m, 2H), | |
| | 1.90-1.79 (m, 4H), 1.38 (s, 3H), 1.19 (t, J = 7.2 Hz, 3H). | |
| 475 | ¹H NMR (400 MHz, methanol-d₄) δ 8.17 (s, 1H), 8.12 (s, 1H), | 560.1 |
| | 8.03 (s, 1H), 7.76-7.72 (m, 1H), 7.49 (s, 1H), 7.40 (t, J = 8.0 | |
| | Hz, 1H), 6.84 (d, J = 8.0 Hz, 1H), 5.08 (s, 2H), 4.36-4.31 (m, | |
| | 1H), 3.37 (s, 2H), 3.35-3.31 (m, 1H), 3.31-3.27 (m, 1H), | |
| | 3.18-3.13 (m, 1H), 3.06-3.02 (m, 2H), 2.78 (s, 3H), 2.35- | |
| | 2.25 (m, 1H), 2.15-2.05 (m, 1H), 1.80-1.54 (m, 4H), 1.02 | |
| | (t, J = 7.6 Hz, 3H). | |
| 476 | ¹H NMR (400 MHz, methanol-d₄) δ 8.17 (s, 1H), 8.12 (s, 1H), | 560.2 |
| | 8.03 (s, 1H), 7.73-7.72 (m, 1H), 7.49 (t, J = 2.0 Hz, 1H), 7.40 | |
| | (t, J = 8.0 Hz, 1H), 6.86-6.83 (m, 1H), 5.08 (s, 2H), 4.36- | |
| | 4.31 (m, 1H), 3.37 (s, 2H), 3.35-3.31 (m, 1H), 3.31-3.27 | |
| | (m, 1H), 3.18-3.13 (m, 1H), 3.06-3.02 (m, 2H), 2.78 (s, | |
| | 3H), 2.35-2.25 (m, 1H), 2.15-2.05 (m, 1H), 1.80-1.55 (m, | |
| | 4H), 1.02 (t, J = 7.2 Hz, 3H). | |
| 477 | ¹H NMR (400 MHz, methanol-d₄) δ 8.19 (s, 1H), 8.05 (s, 1H), | 643.2 |
| | 7.98 (s, 1H), 7.04 (s, 1H), 6.62 (s, 1H), 5.94 (s, 1H), 5.00 (s, | |
| | 2H), 3.85 (s, 2H), 3.68-3.66 (m, 1H), 3.31-3.30 (m, 2H), | |
| | 3.26-3.18 (m, 2H), 2.98-2.94 (m, 2H), 2.86 (s, 3H), 2.10- | |
| | 2.06 (m, 2H), 1.95-1.78 (m, 8H), 1.65-1.55 (m, 2H), 1.50- | |
| | 1.40 (m, 2H), 1.37 (s, 3H). | |
| 478 | ¹H NMR (400 MHz, methanol-d₄) δ 8.21 (s, 1H), 8.09 (s, 1H), | 644.4 |
| | 8.02 (s, 1H), 7.46 (s, 1H), 6.96 (s, 1H), 6.28 (s, 1H), 5.09 (s, | |
| | 2H), 4.78-4.75 (m, 1H), 3.89 (s, 2H), 3.39-3.36 (m, 4H), | |
| | 3.08-3.00 (m, 2H), 2.87 (s, 3H), 2.20-2.10 (m, 2H), 1.93-1.87 | |
| | (m, 4H), 1.81-1.78 (m, 6H), 1.77-1.60 (m, 2H), 1.40 (s, 3H). | |
| 479 | ¹H NMR (400 MHz, methanol-d₄) δ 8.24 (s, 1H), 8.10 (s, 1H), | 610.3 |
| | 8.03 (s, 1H), 7.50 (t, J = 2.4 Hz, 1H), 6.85 (d, J = 1.6 Hz, 1H), | |
| | 6.21 (s, 1H), 5.09-5.06 (m, 6H), 4.79-4.76 (m, 1H), 3.89 | |
| | (s, 2H), 3.66 (s, 2H), 2.98 (s, 3H), 2.14-2.11 (m, 2H), 1.94- | |
| | 1.78 (m, 10H), 1.67-1.65 (m, 2H), 1.41 (s, 3H). | |
| 480 | ¹H NMR (400 MHz, methanol-d₄) δ 8.22 (s, 1H), 8.07 (s, 1H), | 609.3 |
| | 8.00 (s, 1H), 7.08 (t, J = 2.0 Hz, 1H), 6.49 (d, J = 1.2 Hz, 1H), | |
| | 5.82 (s, 1H), 5.02 (s, 6H), 3.86 (s, 2H), 3.71-3.64 (m, 1H), | |
| | 3.62 (s, 2H), 2.95 (s, 3H), 2.12-2.08 (m, 2H), 1.92-1.89 (m, | |
| | 4H), 1.86-1.79 (m, 4H), 1.73-1.61 (m, 2H), 1.44-1.38 (m, | |
| | 2H), 1.38 (s, 3H). | |
| 481 | ¹H NMR (400 MHz, methanol-d₄) δ 8.22 (s, 1H), 8.06 (d, J = | |
| | 7.6 Hz, 1H), 7.95 (d, J = 7.6 Hz, 1H), 7.74 (t, J = 8.0 Hz, 1H), | |
| | 7.27 (s, 1H), 5.73 (s, 1H), 5.21 (s, 2H), 5.02-5.00 (m, 2H), | |
| | 4.98-4.95 (m, 2H), 3.82-3.76 (m, 1H), 3.62 (s, 2H), 3.15 | |
| | (s, 3H), 1.63-1.57 (m, 1H), 1.50-1.46 (m, 1H), 1.16 (d, J = | |
| | 6.4 Hz, 3H), 0.94 (t, J = 7.6 Hz, 3H). | |
| 482 | ¹H NMR (400 MHz, methanol-d₄) δ 8.22 (s, 1H), 8.06 (d, J = | 501.2 |
| | 8.0 Hz, 1H), 7.95 (d, J = 7.6 Hz, 1H), 7.74 (t, J = 8.0 Hz, 1H), | |
| | 7.27 (s, 1H), 5.73 (s, 1H), 5.21 (s, 2H), 5.02-5.00 (m, 2H), | |
| | 4.98-4.95 (m, 2H), 3.82-3.76 (m, 1H), 3.62 (s, 2H), 3.15 | |
| | (s, 3H), 1.61-1.57 (m, 1H), 1.52-1.46 (m, 1H), 1.16 (d, J = | |
| | 6.4 Hz, 3H), 0.94 (t, J = 7.6 Hz, 3H). | |
| 483 | ¹H NMR (400 MHz, DMSO-d₆) δ 8.25 (s, 1H), 8.07-8.02 (m, | 549.3 |
| | 2H), 7.81-7.76 (m, 1H), 7.48 (s, 1H), 6.93 (d, J = 6.0 Hz, | |
| | 1H), 6.01 (s, 1H), 5.19 (s, 2H), 4.91-4.89 (m, 2H), 4.80- | |
| | 4.77 (m, 2H), 4.23-4.15 (m, 1H), 3.50 (s, 2H), 3.25 (s, 3H), | |
| | 2.66-2.57 (m, 1H), 2.27-2.03 (m, 4H), 1.79-1.73 (m, 1H). | |
| 484 | ¹H NMR (400 MHz, DMSO-d₆) δ 8.26 (s, 1H), 8.07-8.02 (m, | 511.3 |
| | 2H), 7.80-7.76 (m, 1H), 7.43 (s, 1H), 6.83 (d, J = 6.4 Hz, | |
| | 1H), 5.95 (s, 1H), 5.74 (s, 2H), 5.19 (s, 2H), 4.89 (d, J = 6.0 | |
| | Hz, 2H), 4.78 (d, J = 6.0 Hz, 2H), 4.34-4.32 (m, 1H), 3.49 | |
| | (s, 2H), 3.23 (s, 3H), 2.75-2.67 (m, 2H), 2.27-2.21 (m, 2H). | |
| 485 | ¹H NMR (400 MHz, DMSO-d6) δ 8.43 (s, 1H), 7.99 (s, 1H), | 649.3 |
| | 7.96-7.82 (m, 2H), 7.59 (d, J = 2.3 Hz, 1H), 7.35 (t, J = 8.0 | |
| | Hz, 1H), 7.05 (d, J = 7.9 Hz, 1H), 6.14 (d, J = 45.2 Hz, 1H), | |
| | 5.19-4.99 (m, 2H), 4.22 (d, J = 14.4 Hz, 1H), 3.79-3.33 (m, | |
| | 5H), 3.09 (s, 1H), 2.73-2.55 (m, 2H), 2.36-2.18 (m, 4H), | |
| | 2.14 (s, 3H), 1.94 (q, J = 10.6 Hz, 2H), 1.13-0.69 (m, 10H). | |
| 486 | ¹H NMR (400 MHz, DMSO-d6) δ 8.40 (s, 1H), 8.00-7.88 (m, | 627.3 |
| | 3H), 7.66 (t, J = 1.9 Hz, 1H), 7.35 (t, J = 8.0 Hz, 1H), 7.04- | |
| | 6.97 (m, 1H), 6.23 (d, J = 46.4 Hz, 1H), 5.50 (d, J = 6.8 Hz, | |
| | 1H), 5.30 (dd, J = 6.3, 1.9 Hz, 1H), 5.15 (dd, J = 6.8, 2.2 Hz, | |
| | 1H), 5.12-5.04 (m, 3H), 4.83 (dd, J = 6.3, 3.5 Hz, 1H), 4.21 | |
| | (d, J = 14.4 Hz, 1H), 3.38 (d, J = 14.4 Hz, 2H), 2.83 (tt, J = | |
| | 7.3, 3.9 Hz, 1H), 2.61 (t, J = 12.1 Hz, 2H), 2.42-2.18 (m, | |
| | 3H), 2.14 (s, 3H), 1.99-1.87 (m, 2H), 0.97-0.75 (m, 9H), | |
| | 0.38 (ddd, J = 10.4, 8.5, 4.9 Hz, 1H). | |
| 487 | ¹H NMR (400 MHz, DMSO-d6) δ 8.40 (s, 1H), 8.00-7.88 (m, | 627.3 |
| | 3H), 7.66 (t, J = 1.9 Hz, 1H), 7.35 (t, J = 8.0 Hz, 1H), 7.04- | |
| | 6.97 (m, 1H), 6.23 (d, J = 46.4 Hz, 1H), 5.50 (d, J = 6.8 Hz, | |
| | 1H), 5.30 (dd, J = 6.3, 1.9 Hz, 1H), 5.15 (dd, J = 6.8, 2.2 Hz, | |
| | 1H), 5.12-5.04 (m, 3H), 4.83 (dd, J = 6.3, 3.5 Hz, 1H), 4.21 | |
| | (d, J = 14.4 Hz, 1H), 3.38 (d, J = 14.4 Hz, 2H), 2.83 (tt, J = | |
| | 7.3, 3.9 Hz, 1H), 2.61 (t, J = 12.1 Hz, 2H), 2.42-2.18 (m, | |
| | 3H), 2.14 (s, 3H), 1.99-1.87 (m, 2H), 0.97-0.75 (m, 9H), | |
| | 0.38 (ddd, J = 10.4, 8.5, 4.9 Hz, 1H). | |
| 488 | ¹H NMR (400 MHz, DMSO-d6) δ 8.24 (s, 1H), 8.18-7.91 (m, | 541.1 |
| | 2H), 7.79 (t, J = 7.8 Hz, 1H), 7.49 (s, 1H), 6.91 (t, J = 5.8 Hz, | |
| | 1H), 6.01 (s, 1H), 5.21 (s, 2H), 5.10-4.67 (m, 4H), 3.49 (d, J = | |
| | 5.1 Hz, 4H), 3.24 (s, 3H), 2.57 (dd, J = 13.5, 5.9 Hz, 2H). | |
| 489 | (No Data) | 529.1 |
| 490 | ¹H NMR (400 MHz, DMSO-d6) δ 8.23 (s, 1H), 8.06 (dd, J = | 529.1 |
| | 11.9, 7.7 Hz, 2H), 7.79 (t, J = 7.7 Hz, 1H), 7.55 (s, 1H), 6.13 | |
| | (s, 1H), 5.19 (s, 2H), 4.92 (d, J = 6.1 Hz, 2H), 4.84 (dd, J = | |
| | 10.8, 6.1 Hz, 2H), 4.22 (d, J = 7.3 Hz, 1H), 4.00-3.86 (m, | |
| | 1H), 3.83-3.70 (m, 2H), 3.61 (dd, J = 11.3, 3.0 Hz, 1H), 3.58- | |
| | 3.49 (m, 2H), 3.47 (td, J = 11.6, 3.0 Hz, 1H), 3.17 (s, 3H), | |
| | 3.01 (td, J = 12.7, 3.8 Hz, 1H), 1.08 (d, J = 6.6 Hz, 3H). | |
| 491 | ¹H NMR (400 MHz, DMSO-d6) δ 8.23 (s, 1H), 8.05 (dd, J = | 502.1 |
| | 9.9, 7.7 Hz, 2H), 7.79 (t, J = 7.7 Hz, 1H), 7.45 (d, J = 1.2 Hz, | |
| | 1H), 7.29 (s, 1H), 6.95 (s, 1H), 6.84 (t, J = 5.9 Hz, 1H), 6.07 | |
| | (s, 1H), 5.15 (s, 2H), 4.91 (d, J = 6.0 Hz, 2H), 4.78 (d, J = 6.1 | |
| | Hz, 2H), 3.78 (d, J = 5.7 Hz, 2H), 3.49 (s, 2H), 3.23 (s, 3H). | |
| 492 | ¹H NMR (400 MHz, DMSO-d6) δ 8.21 (s, 1H), 8.05 (s, 1H), | 586.2 |
| | 8.01 (s, 1H), 7.84 (dd, J = 7.8, 2.3 Hz, 1H), 7.56 (t, J = 2.0 Hz, | |
| | 1H), 7.32 (t, J = 8.0 Hz, 1H), 6.86-6.79 (m, 1H), 5.09 (s, 2H), | |
| | 3.82 (s, 2H), 3.36 (s, 3H), 3.34 (d, J = 4.6 Hz, 1H), 3.06 (q, J = | |
| | 13.9 Hz, 2H), 2.10-1.88 (m, 3H), 1.78-1.61 (m, 4H), 1.23 | |
| | (s, 3H), 0.72 (td, J = 7.3, 5.1 Hz, 2H), 0.62-0.54 (m, 2H). | |
| 493 | ¹H NMR (400 MHz, DMSO-d6) δ 8.27 (s, 1H), 8.04 (dd, J = | 525.2 |
| | 10.7, 7.7 Hz, 2H), 7.78 (t, J = 7.7 Hz, 1H), 7.48 (d, J = 1.3 Hz, | |
| | 1H), 7.18 (s, 1H), 6.02 (d, J = 1.2 Hz, 1H), 5.15 (s, 2H), 4.89 | |
| | (d, J = 6.0 Hz, 2H), 4.77 (d, J = 6.0 Hz, 2H), 3.49 (s, 2H), 3.29 | |
| | (s, 3H), 2.33 (q, J = 2.4, 1.7 Hz, 1H), 1.88 (s, 2H), 1.84-1.76 | |
| | (m, 2H), 1.69-1.61 (m, 2H), 1.42 (dd, J = 3.8, 1.9 Hz, 2H). | |
| 494 | ¹H NMR(400 MHz, dmso) δ 8.35 (s, 1H), 8.25 (s, 1H), 8.04 | 544.3 |
| | (d, J = 18.1 Hz, 2H), 7.95 (dd, J = 8.1, 1.5 Hz, 1H), 7.55 (t, J = | |
| | 1.8 Hz, 1H), 7.38 (t, J = 8.0 Hz, 1H), 6.97 (d, J = 7.8 Hz, 1H), | |
| | 6.28 (d, J = 45.9 Hz, 1H), 5.37 (d, J = 6.5 Hz, 1H), 5.22 (d, J = | |
| | 5.4 Hz, 1H), 5.17-5.03 (m, 3H), 4.83 (dd, J = 6.2, 4.0 Hz, | |
| | 1H), 3.83 (s, 2H), 3.19 (s, 3H), 2.04-1.93 (m, 2H), 1.77- | |
| | 1.63 (m, 4H), 1.23 (s, 3H). | |

### Example 76: Cbl-b and C-Cbl LCK Ub TR-FRET Assay

Compounds were 3-fold serially diluted in DMSO in a 384-well polypropylene plate (#P-05525-BC; Labcyte) to generate a source plate with 10 concentrations of each compound, top concentration=2 mM. 80 nL of DMSO or compounds were transferred to each well of a black 384-well ProxiPlate (#6008260; PerkinElmer) using a Labcyte Echo. 1×assay buffer (50 mM HEPES pH7.0, 100 mM NaCl, 0.01% BSA, 0.01% Triton-X100, 1 mM DTT), 2×enzyme solution (16 nM Biotin-Cbl-b or 12 nM Biotin-c-Cbl in 1× assay buffer), 2×kinase mixture (120 nM His-LCK, 1 mM ATP, 10 mM MgCl₂ in assay buffer) and 2.33×detection mixture (4.66×solution 1: 163 nM Anti-HA-D2 antibody (#610HADAB; PerkinElmer), 27.96 nM Streptavidin-EU (#AD0062; PerkinElmer), 1.398 mM EDTA in 1×assay buffer+4.66×solution 2: 2.796 μM UBE2D2/Methylated-HA-Ubiquitin thioester adduct (BostonBiochem) in 1×assay buffer) were prepared. 4 μL of 2× enzyme solution was added to each well containing compound, briefly centrifuged to mix, and incubated for 60 min at room temperature. 4 μL of 2× kinase mixture was added, briefly centrifuged to mix, and incubated for 90 min. at room temperature. 6 μL of detection mixture was added to all wells and briefly centrifuged before incubating for 20 min at room temperature. Plates were read for TR-FRET using an Envision at excitation 340 nm, emission at 615 and 665 nm, 4 flashes per well. IC₅₀ was generated using no LCK as the low control and DMSO as the high control.

TABLE 3
Selectivity Data for Exemplary Isoindolin-1-one Compounds

| | Compound | Compound | Compound |
|---|---|---|---|
| | 129 | 156 | 201 |
| cbl-b Lck HTRF | 0.13 | 0.92 | 0.22 |
| IC₅₀ (μM) | | | |
| c-cbl Lck HTRF | 0.46 | 5.2 | 3.0 |
| IC₅₀ (μM) | | | |
| fold of selectivity | 3.6x | 5.6x | 13.6x |

### Example 77: PBMC IL-2 Assay

Immune response to compounds described herein can be assessed via a PBMC IL-2 assay, conducted according to the following protocol. PBMCs (#A19K379053, A19K261022; TPCS) are thawed into complete medium: 1640 medium (#2085568; Gibco), 10% FBS (#SH30084.03; HyClone), and 1× pen/strep. Compounds are 3-fold serially diluted in DMSO in a 384-well polypropylene plate (#P-05525-BC; Labcyte) using the Tecan EVO to generate a source plate with 10 concentrations of each compound, top concentration=4 mM. Compounds are dispensed into a 96-well plate (#6005680; PerkinElmer) using a Labcyte Echo; final dispensed volume of each control and compound is 1000 nL (final DMSO=0.5%). After recovery overnight, cells are seeded at 2×10⁵ cells/well into 96-well plates containing compounds and incubated at 37° C., 5% CO₂ for 30 min. Cells are stimulated by adding 20 μL/well 1/10 TransAct (#130-111-160; Miltenyi) diluted in complete medium, placed on a shaker for 2 min at 600 rpm, and incubated for 24 h at 37° C., 5% CO₂. Plates are centrifuged at 1200 rpm for 5 min and 120 μL cell supernatant collected. Supernatants are diluted 10-fold and IL-2 concentrations of each sample are determined using the IL-2 MSD kit (#K151AHB-4; MSD) per the manufacturer's instructions.

### Example 78: Liver Microsomes Metabolic Stability Assays

Metabolic stability of test compounds was evaluated in pooled rat, mouse, dog, and cynomolgus monkey liver microsomes (BD Biosciences, San Jose, CA). The incubation conditions were as follows: 1 μM of the tested compound, 1 mM NADPH, 0.5 mg/mL microsomal protein in 0.1 M potassium phosphate buffer (pH 7.4). Following a 5-minute pre-incubation period, the enzymatic reactions were initiated by the addition of NADPH and test compound to the microsomes diluted in phosphate buffered saline. The mixtures were incubated at 37° C. for 0, 20, 40, and 60 min.

Compound concentrations were assessed by LC-MS/MS. Intrinsic clearance based upon microsomal stability data was determined using a substrate depletion method and scaled to hepatic clearance using the well-stirred model (Obach, R. S.; Baxter, J. G.; Liston, T. E.; Silber, B. M.; Jones, B. C.; Macintyre, F.; Rance, D. J.; Wastall, P., “The Prediction of Human Pharmacokinetic Parameters from Preclinical and in vitro Metabolism Data”, J. Pharmacol. Exp. Ther., (1997), 283 (1), 46-58).

### Example 79: Hepatocyte Metabolic Stability Assays

Metabolic stability assays of test compounds were evaluated in cryopreserved pooled rat, mouse, dog, and cynomolgus monkey hepatocytes (CellzDirect; Durham, NC, USA). Membrane integrity of the cells was assessed by trypan blue exclusion. Test compounds (1.0 μM with 0.1% dimethylsulfoxide) were incubated with cells (0.5 million cells/mL) at 37° C. in a 95% air/5% CO₂ atmosphere for 0, 20, 40, or 60 minutes. Concentrations of test compounds in hepatocyte incubations were determined by LC/MS/MS. Intrinsic clearance was determined using a substrate depletion method and scaled to hepatic clearance using the well-stirred model as described above for the liver microsomes metabolic stability assays.

For illustration purpose, the liver microsomes and hepatocyte stability data for selected compounds from this application are shown below.

TABLE 4
Liver Microsome and Hepatocyte Data for
Representative Isoindolin-1-one Compounds

| | Compound | Compound | Compound |
|---|---|---|---|
| | 68 | 74 | 86 |
| Liver microsomes Pred. CLₕₑₚ | 6/14/12 | 15/37/19 | 7/27/14 |
| (mL/min/kg) | | | |
| Rat/Mouse/Dog | | | |
| Hepatocyte Pred. CLₕₑₚ | 19/37/— | 11/35/— | 12/36/— |
| (mL/min/kg) | | | |
| Rat/Mouse/Dog | | | |

### Example 80: In Vitro Plasma Protein Binding

In vitro plasma protein binding (n=2) was determined in pooled mouse, rat, and human plasma (Bioreclamation, Inc., Hicksville, NY) by equilibrium dialysis using a Rapid Equilibrium Dialysis (RED) device (Pierce Biotechnology/Thermo Fisher Scientific; Rockford, IL) with a molecular weight cut-off of 8000 Daltons. Test compounds were added to plasma. Plasma samples were equilibrated with phosphate-buffered saline at 37° C. for 4 hours. Compound concentrations in post-dialysis plasma and buffer samples were measured by LC-MS/MS. The percent unbound fraction in plasma for each compound was calculated by dividing the compound concentration in the post-dialysis buffer by that measured in the post-dialysis plasma and multiplying by 100%.

### Example 81: In Vitro Permeability Assay in gMDCK (Madin-Darby Canine Kidney) Cells

The permeability of test compounds was determined in gMDCK cells (American Type Culture Collection; Manassas, VA), and is shown in Table 5, which establishes that these compounds have significantly improved permeability than those previously known. Four days prior to use, MDCK cells were seeded at a density of 2.5×10⁵ cells/mL in 24 well plates. Compounds were dissolved in transport buffer consisting of Hank's Balanced Salt Solution with 10 mM HEPES (Invitrogen Corporation, Grand Island, NY) at a concentration of 10 μM, and permeability was assessed in the apical to basolateral (A-B) and basolateral to apical (B-A) directions following a 3 hour incubation. Lucifer Yellow (Sigma Aldrich, St. Louis, MO) was used as the cell monolayer integrity marker. Test compound concentrations in the donor and receiving compartments were determined by LC-MS/MS. The apparent permeability (Pₐₚₚ) of test compounds was determined as follows:

$${P}_{app} = {( dQ / dt )}^{*} ( 1 / {AC}_{0} )$$

Where dQ/dt is the rate of compound appearance in the receiver compartment, Q is the quantity of compound), C₀ is the concentration in the donor compartment and A is the surface area of the insert. Efflux ratio was calculated as P_{app, B-A}/P_{app, A-B}.

TABLE 5
Permeability Data for Representative Isoindolin-1-one Compounds

| | Example 520 from | Compound | Compound |
|---|---|---|---|
| | WO2019148005 | 27 | 127 |
| gMDCK P_{app A-B} | 0.96 (1.17) | 16.3 (0.64) | 14.1 (0.72) |
| (ratio) | | | |

### Example 82: Reversible CYP Inhibition

Reversible CYP inhibition by compounds described herein can be measured by protocols described by Halladay, J. S.; Delarosa, E. M.; Tran, D.; Wang, L.; Wong, S.; Khojasteh, S. C., “High-Throughput, 384-Well, LC-MS/MS CYP Inhibition Assay Using Automation, Cassette-Analysis Technique, and Streamlined Data Analysis”, Drug. Metab. Lett. 2011, 5 (3), 220-230, incorporated herein by reference.

### Example 83: CYP3A Time-Dependent Inhibition (TDI)

Time-dependent inhibition by compounds described herein can be measured by various methods. Exemplary such protocols for CYP3A automated AUC shift dilution TDI assay and definitive K_{I}/K_{inact} TDI assay are described by Kenny, J. R.; Mukadam, S.; Zhang, C.; Tay, S.; Collins, C.; Galetin, A.; Khojasteh, S. C., “Drug-Drug Interaction Potential of Marketed Oncology Drugs: in vitro Assessment of Time-Dependent Cytochrome P450 Inhibition, Reactive Metabolite Formation and Drug-Drug Interaction Prediction,” Pharm. Res. 2012, 29 (7), 1960-1976.

### Example 84: In Vivo Pharmacokinetics (PK)

The pharmacokinetics of test compounds were evaluated following a single intravenous bolus (IV) of solution at a dose of 0.2-1 mg/kg and oral administration (PO) of solution/suspension at doses of 1-5 mg/kg in male cynomolgus monkey, beagle dogs, Sprague Dawley rats, or CD-1 mice using a parallel study design. Blood samples for the IV dose group were collected prior to administration (predose) and at 0.033, 0.083, 0.25, 0.5, 1, 3, 6, 9 and 24 hours post dose. Blood samples for PO dose groups were collected prior to administration (predose) and at 0.083, 0.25, 0.5, 1, 3, 6, 9 and 24 hours post dose. For the IV group, urine was collected from each animal at predose and from 0-6 and 6-24 hours post dose. The vehicle used for IV dose groups was a combination of PEG400 with citrate buffer (pH 5.0) or PEG400/Cremphor with DMSO/H₂O, and for PO groups was MCT. Results are shown in Table 6.

Plasma and urine concentrations were quantitated at Genentech Inc. using a non-validated LC/MS/MS method. The lower limit of quantitation (LLOQ) of the plasma and urine assays was 0.005 μM. PK parameters were determined by non-compartmental methods using WinNonlin (version 5.2, Pharsight Corporation, Mountain View, CA).

TABLE 6
Mouse PK Data for Representative Isoindolin-1-one Compounds

| | Example | Compound | Compound | Compound |
|---|---|---|---|---|
| | 70 | 86 | 187 | 189 |
| Mouse CL | 44 | 40 | 42 | 34 |
| (mL/min/kg) | | | | |
| Oral | 2% | 2% | 13% | 21% |
| Bioavailability | | | | |
| (F %) | | | | |

### Example 85: Pharmacodynamics (PD) Study; Enhancement of CD4 and CD8 T Cell Activation in Response to Systemic Anti-CD3 Administration in the Presence of Cbl-b Inhibitor

Female C57BL/6 or Balb/c mice are administered with anti-CD3 antibody (2 ug/mouse, clone 2C11) or an isotype control (2 μg/mouse, hamster IgG) is administered by tail vein injection. A Cbl-b inhibitor is administered PO starting at time 0 (immediately before anti-CD3 administration) and again 8 hrs later. Four hours after anti-CD3 administration, mice are bled and cytokines are quantified in serum via Luminex. Twenty-four hours after anti-CD3 administration, mice are euthanized and activation of CD4 and CD8 T cells quantified in spleens and blood. Expression of 4-1 BB, CD25, CD40L, and CD69 as well as cell surface TCR levels will be quantified by flow cytometry. Serum are collected for cytokine analysis via Luminex.

### Example 86: Tumor PD/Efficacy Study; Evaluation of Tumor Growth and Immune Cell Infiltration in Mice with Syngeneic Tumors Treated Prophylactically or Therapeutically with Cbl-b Inhibitor

Female C57Bl/6 mice age 6-12 weeks are inoculated subcutaneously in the 5th mammary fat pad with 0.1 million E0771 cells in 100 microliters of HBSS+matrigel under manual restraint. For prophylactic studies, a Cbl-b inhibitor is administered PO BID×3 weeks starting 1 hr prior to tumor inoculation. Three weeks after tumor inoculation, mice are euthanized and tumor, spleen, blood and draining lymph node are harvested and immune cell infiltrate and phenotype are assessed by flow cytometry. Serum are obtained at various time points for cytokine analysis via Luminex. For therapeutic efficacy assessment, tumors are inoculated as described above and allowed to grow until tumors reach a median volume of 120-250 mm³. Dosing with a Cbl-b inhibitor is then be initiated as above and continued until end of study. Tumor volumes and mouse body weight and condition are recorded twice weekly or more as needed until end of study. Efficacy of a Cbl-b inhibitor can also be assessed in additional syngeneic tumor models, including CT₂₆ and TC-1.

TABLE 7
Cbl-b and C-Cbl Selectivity of Isoindolin-1-one Compounds

| | Cbl-b Lck | C-cbl Lck | |
|---|---|---|---|
| | HTRF IC₅₀ | HTRF IC₅₀ | (fold- |
| Compound | (μM) | (μM) | selectivity) |
| 9 | 0.16 | 0.35 | 2.1x |
| 10 | 0.50 | 1.1 | 2.3x |
| 11 | 0.42 | 0.62 | 1.5x |
| 12 | 0.22 | 0.21 | 0.94x |
| 13 | 0.32 | 0.54 | 1.7x |
| 14 | 10 | 20 | 2.0x |
| 15 | 3.5 | 6.4 | 1.8x |
| 17 | 0.017 | 0.014 | 0.85x |
| 27 | 0.039 | 0.061 | 1.6x |
| 28 | 0.080 | 0.18 | 2.2x |
| 29 | 0.12 | 0.23 | 1.8x |
| 30 | 0.065 | 0.091 | 1.4x |
| 31 | 0.088 | 0.13 | 1.4x |
| 32 | 4.5 | 11.0 | 2.5x |
| 33 | 0.89 | 1.9 | 2.1x |
| 34 | 0.38 | 0.76 | 2.0x |
| 47 | 0.10 | 0.18 | 1.8x |
| 48 | 0.32 | 0.78 | 2.4x |
| 49 | 1.4 | 2.8 | 2.0x |
| 50 | 0.054 | 0.090 | 1.7x |
| 51 | 0.062 | 0.15 | 2.4x |
| 52 | 0.062 | 0.13 | 2.1x |
| 53 | 0.034 | 0.063 | 1.9x |
| 54 | 0.029 | 0.046 | 1.6x |
| 55 | 0.016 | 0.026 | 1.6x |
| 56 | 0.016 | 0.022 | 1.4x |
| 57 | 0.049 | 0.067 | 1.4x |
| 58 | 0.20 | 0.36 | 1.8x |
| 59 | 0.59 | 1.3 | 2.1x |
| 66 | 0.076 | 0.10 | 1.3x |
| 68 | 0.079 | 0.11 | 1.3x |
| 69 | 0.21 | 0.34 | 1.6x |
| 70 | 0.11 | 0.21 | 1.9x |
| 71 | 0.24 | 0.40 | 1.7x |
| 72 | 0.055 | 0.11 | 2.1x |
| 73 | 0.14 | 0.30 | 2.2x |
| 74 | 0.057 | 0.12 | 2.2x |
| 75 | 0.25 | 0.58 | 2.3x |
| 76 | 0.052 | 0.082 | 1.6x |
| 77 | 0.21 | 0.37 | 1.8x |
| 78 | 0.053 | 0.079 | 1.5x |
| 79 | 0.17 | 0.27 | 1.6x |
| 80 | 0.11 | 0.29 | 2.6x |
| 81 | 0.27 | 0.75 | 2.8x |
| 82 | 0.028 | 0.068 | 2.4x |
| 83 | 0.21 | 0.64 | 3.1x |
| 84 | 0.062 | 0.12 | 1.9x |
| 85 | 0.18 | 0.37 | 2.0x |
| 86 | 0.13 | 0.27 | 2.1x |
| 87 | 1.2 | 3.1 | 2.6x |
| 88 | 0.10 | 0.21 | 2.1x |
| 89 | 0.31 | 0.72 | 2.3x |
| 90 | 0.042 | 0.058 | 1.4x |
| 91 | 0.14 | 0.17 | 1.2x |
| 92 | 0.020 | 0.024 | 1.2x |
| 93 | 0.068 | 0.078 | 1.1x |
| 94 | 0.18 | 0.20 | 1.1x |
| 95 | 0.27 | 0.41 | 1.5x |
| 96 | 0.012 | 0.018 | 1.5x |
| 97 | 0.020 | 0.038 | 1.9x |
| 99 | 0.17 | 0.34 | 2.0x |
| 100 | 0.19 | 0.38 | 1.9x |
| 101 | 0.018 | 0.023 | 1.2x |
| 102 | 0.018 | 0.025 | 1.4x |
| 103 | 0.015 | 0.022 | 1.5x |
| 104 | 0.048 | 0.089 | 1.9x |
| 105 | 0.026 | 0.025 | 0.93x |
| 106 | 0.018 | 0.015 | 0.81x |
| 107 | 0.018 | 0.019 | 1.0x |
| 108 | 0.083 | 0.16 | 2.0x |
| 109 | 0.044 | 0.085 | 1.9x |
| 110 | 0.32 | 0.65 | 2.0x |
| 111 | 0.19 | 0.34 | 1.8x |
| 116 | 0.31 | 0.33 | 1.1x |
| 117 | 0.54 | 0.75 | 1.4x |
| 118 | 0.007 | 0.008 | 1.1x |
| 119 | 0.048 | 0.052 | 1.1x |
| 120 | 0.036 | 0.044 | 1.2x |
| 125 | 0.14 | 0.24 | 1.7x |
| 126 | 0.064 | 0.093 | 1.5x |
| 127 | 0.077 | 0.17 | 2.2x |
| 128 | 0.48 | 1.6 | 3.4x |
| 129 | 0.13 | 0.46 | 3.6x |
| 130 | 4.0 | 13 | 3.3x |
| 131 | 0.059 | 0.17 | 2.9x |
| 132 | 0.23 | 0.47 | 2.1x |
| 133 | 0.090 | 0.17 | 1.9x |
| 134 | 0.21 | 0.67 | 3.3x |
| 135 | 0.096 | 0.18 | 1.9x |
| 136 | 0.28 | 0.56 | 2.0x |
| 137 | 0.029 | 0.057 | 1.9x |
| 138 | 0.098 | 0.18 | 1.8x |
| 139 | 0.23 | 0.64 | 2.8x |
| 140 | 0.38 | 0.97 | 2.8x |
| 141 | 0.091 | 0.12 | 1.3x |
| 142 | 0.23 | 0.43 | 1.9x |
| 143 | 0.029 | 0.027 | 0.92x |
| 144 | 0.072 | 0.066 | 0.91x |
| 145 | 0.13 | 0.18 | 1.4x |
| 146 | 0.43 | 0.48 | 1.1x |
| 147 | 0.15 | 0.19 | 1.3x |
| 148 | 0.69 | 0.73 | 1.1x |
| 149 | 0.38 | 0.39 | 1.0x |
| 150 | 1.4 | 1.5 | 1.1x |
| 151 | 0.061 | 0.085 | 1.4x |
| 152 | 0.22 | 0.28 | 1.3x |
| 153 | 0.027 | 0.060 | 2.2x |
| 154 | 0.072 | 0.23 | 3.2x |
| 155 | 0.13 | 0.24 | 1.8x |
| 156 | 0.92 | 5.2 | 5.6x |
| 157 | 0.072 | 0.11 | 1.6x |
| 158 | 0.27 | 0.28 | 1.0x |
| 159 | 0.35 | 1.2 | 3.4x |
| 160 | 1.4 | 2.3 | 1.7x |
| 161 | 0.062 | 0.11 | 1.7x |
| 162 | 0.19 | 0.33 | 1.7x |
| 163 | 0.13 | 0.24 | 1.9x |
| 164 | 0.35 | 0.64 | 1.8x |
| 165 | 0.035 | 0.073 | 2.1x |
| 166 | 0.17 | 0.31 | 1.8x |
| 167 | 0.059 | 0.15 | 2.5x |
| 168 | 0.18 | 0.38 | 2.1x |
| 169 | 0.044 | 0.099 | 2.2x |
| 170 | 0.14 | 0.36 | 2.5x |
| 171 | 0.049 | 0.082 | 1.7x |
| 172 | 0.18 | 0.52 | 2.9x |
| 173 | 0.063 | 0.083 | 1.3x |
| 174 | 0.19 | 0.44 | 2.3x |
| 175 | 0.15 | 0.35 | 2.5x |
| 176 | 0.35 | 1.3 | 3.5x |
| 177 | 0.041 | 0.091 | 2.2x |
| 178 | 0.17 | 0.35 | 2.0x |
| 179 | 0.054 | 0.11 | 2.1x |
| 180 | 0.22 | 0.43 | 1.9x |
| 181 | 0.052 | 0.11 | 2.1x |
| 182 | 0.17 | 0.43 | 2.6x |
| 183 | 0.057 | 0.12 | 2.2x |
| 184 | 0.16 | 0.37 | 2.3x |
| 185 | 0.072 | 0.11 | 1.5x |
| 186 | 0.11 | 0.20 | 1.7x |
| 187 | 0.011 | 0.013 | 1.1x |
| 188 | 0.013 | 0.025 | 2.0x |
| 189 | 0.012 | 0.011 | 0.95x |
| 190 | 0.047 | 0.038 | 0.82x |
| 191 | 0.035 | 0.052 | 1.5x |
| 192 | 0.032 | 0.053 | 1.6x |
| 193 | 0.072 | 0.19 | 2.6x |
| 194 | 0.27 | 0.81 | 3.0x |
| 195 | 0.053 | 0.11 | 2.0x |
| 196 | 0.24 | 0.66 | 2.8x |
| 197 | 0.008 | 0.013 | 1.6x |
| 198 | 0.014 | 0.021 | 1.5x |
| 199 | 0.019 | 0.056 | 2.9x |
| 200 | 0.027 | 0.039 | 1.5x |
| 201 | 0.22 | 3.0 (13x) | 13x |
| 202 | 0.15 | 0.40 | 2.6x |
| 203 | 0.20 | 0.40 | 2.0x |
| 204 | 0.21 | 0.66 | 3.1x |
| 205 | 0.29 | 0.77 | 2.7x |
| 206 | 1.5 | 2.3 | 1.5x |
| 207 | 4.5 | 13 | 2.8x |
| 208 | 1.4 | 2.1 | 1.5x |
| 209 | 1.1 | 1.9 | 1.7x |
| 210 | >20 | >20 | 1.0x |
| 211 | 0.94 | 1.6 | 1.7x |
| 212 | 0.075 | 0.13 (1.7) | 1.7x |
| 213 | 0.038 | 0.061 | 1.6x |
| 214 | 0.042 | 0.041 | 0.96x |
| 215 | 0.0075 | 0.0050 | 0.66x |
| 216 | 0.0080 | 0.0048 | 0.60x |
| 217 | 0.017 | 0.020 | 1.2x |
| 218 | 0.022 | 0.029 | 1.3x |
| 219 | 0.075 | 0.084 | 1.1x |
| 220 | 0.034 | 0.035 | 1.0x |
| 221 | 0.015 | 0.015 | 1.0x |
| 222 | 0.022 | 0.026 | 1.2x |
| 226 | 0.029 | 0.033 | (1.1x) |
| 227 | 0.016 | 0.013 | (0.83x) |
| 228 | 0.012 | 0.096 | (0.78x) |
| 229 | 0.012 | 0.010 | (0.83x) |
| 230 | 0.013 | 0.011 | (0.84x) |
| 231 | 0.0098 | 0.081 | (0.82x) |
| 232 | 0.011 | 0.008 | (0.80x) |
| 233 | 0.0083 | 0.0071 | (0.94x) |
| 234 | 0.0072 | 0.0085 | (1.2x) |
| 235 | 0.015 | 0.021 | (1.4x) |
| 236 | 0.0071 | 0.0080 | (1.1x) |
| 237 | 0.10 | 0.18 | (1.8x) |
| 238 | 0.011 | 0.011 | (1.0x) |
| 239 | 0.035 | 0.048 | (1.4x) |
| 240 | 0.054 | 0.039 | (0.71x) |
| 241 | 0.021 | 0.016 | (0.77x) |
| 242 | 0.031 | 0.041 | (1.3x) |
| 243 | 0.029 | 0.047 | (1.6x) |
| 244 | 0.015 | 0.014 | (0.97x) |
| 245 | 0.013 | 0.013 | (1.0x) |
| 246 | 0.0058 | 0.0044 | (0.76x) |
| 247 | 0.0076 | 0.011 | (1.4x) |
| 248 | 0.0067 | 0.0052 | (0.79x) |
| 249 | 0.012 | 0.020 | (1.7x) |
| 250 | 0.012 | 0.0093 | (0.77x) |
| 251 | 0.011 | 0.0084 | (0.76x) |
| 252 | 0.013 | 0.011 | (0.85x) |
| 253 | 1.4 | 3.4 | (2.6x) |
| 254 | 2.4 | 3.2 | (1.4x) |
| 255 | 20 | 20 | (1.0x) |
| 256 | 0.015 | 0.013 | (0.83x) |
| 257 | 13 | 20 | (1.6x) |
| 258 | 0.022 | 0.031 | (1.4x) |
| 259 | 3.1 | 3.1 | (1.0x) |
| 260 | 5.6 | 8.3 | (1.5x) |
| 261 | 0.14 | 0.15 | (1.1x) |
| 262 | 0.58 | 0.84 | (1.4x) |
| 263 | 0.045 | 0.059 | (1.3x) |
| 264 | 7.1 | 11 | (1.5x) |
| 265 | 0.88 | 1.3 | (1.4x) |
| 266 | 0.013 | 0.0097 | (0.76x) |
| 267 | 0.017 | 0.017 | (1.0x) |
| 268 | 0.016 | 0.011 | (0.68x) |
| 269 | 0.21 | 0.47 | (2.3x) |
| 270 | 0.13 | 0.35 | (2.6x) |
| 271 | 1.6 | 1.9 | (1.2x) |
| 272 | 8.2 | 11 | (1.3x) |
| 273 | 0.051 | 0.077 | (1.5x) |
| 274 | 0.14 | 0.22 | (1.6x) |
| 275 | 0.007 | 0.007 | (1.0x) |
| 276 | 0.015 | 0.014 | (0.97x) |
| 277 | 0.23 | 0.87 | (3.8x) |
| 278 | 0.012 | 0.015 | (1.2x) |
| 279 | 0.0079 | 0.010 | (1.4x) |
| 280 | 0.012 | 0.011 | (0.96x) |
| 281 | 0.016 | 0.011 | (0.68x) |
| 282 | 0.013 | 0.011 | (0.86x) |
| 283 | 0.053 | 0.057 | (1.1x) |
| 284 | 0.012 | 0.0074 | (0.59x) |
| 285 | 0.008 | 0.0053 | (0.71x) |
| 286 | 0.0068 | 0.0049 | (0.73x) |
| 287 | 0.032 | 0.043 | (1.4x) |
| 288 | 0.012 | 0.0093 | (0.77x) |
| 289 | 0.012 | 0.020 | (1.7x) |
| 290 | 0.010 | 0.0054 | (0.54x) |
| 291 | 0.012 | 0.0082 | (0.69x) |
| 292 | 3.4 | 5.1 | (1.5x) |
| 293 | 0.0055 | 0.0078 | (1.4x) |
| 294 | 0.0095 | 0.016 | (1.7x) |
| 295 | 0.044 | 0.033 | (0.75x) |
| 296 | 0.44 | 2.5 | (5.7x) |
| 297 | 0.0091 | 0.0080 | (0.87x) |
| 298 | 0.0099 | 0.013 | (1.3x) |
| 299 | 0.011 | 0.0054 | (0.50x) |
| 300 | 0.0056 | 0.0047 | (0.83x) |
| 301 | 0.012 | 0.010 | (0.86x) |
| 302 | 0.59 | 0.66 | (1.1x) |
| 303 | 0.31 | 0.19 | (0.59x) |
| 304 | 0.011 | 0.0079 | (0.73x) |
| 305 | 0.039 | 0.049 | (1.3x) |
| 306 | 0.076 | 0.089 | (1.2x) |
| 307 | 0.18 | 0.31 | (1.7x) |
| 308 | 0.012 | 0.0075 | (0.63x) |
| 309 | 0.54 | 0.52 | (0.97x) |
| 310 | 0.010 | 0.011 | (1.1x) |
| 311 | 0.11 | 0.14 | (1.3x) |
| 312 | 0.07 | 0.11 | (1.5x) |
| 313 | 0.024 | 0.023 | (0.95x) |
| 314 | 0.027 | 0.031 | (1.1x) |
| 315 | 0.57 | 0.48 | (0.84x) |
| 316 | 0.016 | 0.020 | (1.2x) |
| 317 | 0.022 | 0.027 | (1.2x) |
| 318 | 0.13 | 0.14 | (1.1x) |
| 319 | 0.014 | 0.016 | (1.1x) |
| 320 | 0.021 | 0.051 | (2.4x) |
| 321 | 0.018 | 0.023 | (1.3x) |
| 322 | 0.027 | 0.034 | (1.2x) |
| 323 | 0.011 | 0.0084 | (0.76x) |
| 324 | 0.012 | 0.0076 | (0.66x) |
| 325 | 0.047 | 0.041 | (0.87x) |
| 326 | 0.015 | 0.016 | (1.1x) |
| 327 | 0.012 | 0.012 | (1.0x) |
| 328 | 0.015 | 0.021 | (1.4x) |
| 329 | 0.098 | 0.065 | (0.66x) |
| 330 | 1 | 1.2 | (1.2x) |
| 331 | 0.029 | 0.047 | (1.6x) |
| 332 | 0.028 | 0.034 | (1.2x) |
| 333 | 0.052 | 0.053 | (1.0x) |
| 334 | 0.011 | 0.0081 | (0.72) |
| 335 | 0.18 | 0.19 | (1.0x) |
| 336 | 0.033 | 0.023 | (0.70x) |
| 337 | 2.4 | 1.1 | (0.45x) |
| 338 | 0.13 | 0.14 | (1.1x) |
| 339 | 0.53 | 0.36 | (0.68x) |
| 340 | 0.44 | 0.32 | (0.74x) |
| 341 | 0.014 | 0.010 | (0.74x) |
| 342 | 0.011 | 0.0079 | (0.73x) |
| 343 | 0.013 | 0.0095 | (0.74x) |
| 344 | 0.016 | 0.013 | (0.82x) |
| 345 | 0.021 | 0.016 | (0.77x) |
| 346 | 0.011 | 0.010 | (0.92x) |
| 347 | 0.032 | 0.043 | (1.4x) |
| 348 | 0.013 | 0.013 | (1.0x) |
| 349 | 0.0068 | 0.0049 | (0.73x) |
| 350 | 0.028 | 0.016 | (0.57x) |
| 351 | 0.033 | 0.019 | (0.56x) |
| 352 | 0.012 | 0.0067 | (0.56x) |
| 353 | 0.012 | 0.0066 | (0.56x) |
| 354 | 0.013 | 0.0070 | (0.55x) |
| 355 | 0.014 | 0.0091 | (0.66x) |
| 356 | 0.012 | 0.010 | (0.81x) |
| 357 | 0.038 | 0.041 | (1.1x) |
| 358 | 0.016 | 0.015 | (0.91x) |
| 359 | 0.011 | 0.0053 | (0.71x) |
| 360 | 0.020 | 0.017 | (0.86x) |
| 361 | 0.013 | 0.0096 | (0.76x) |
| 362 | 0.012 | 0.0074 | (0.59x) |
| 363 | 0.013 | 0.0096 | (0.75x) |
| 364 | 0.015 | 0.0097 | (0.63x) |
| 365 | 0.012 | 0.0067 | (0.57x) |
| 366 | 0.026 | 0.027 | (1.0x) |
| 367 | 0.030 | 0.029 | (0.96x) |
| 368 | 0.020 | 0.014 | (0.72x) |
| 369 | 0.017 | 0.015 | (0.90x) |
| 370 | 0.062 | 0.063 | (1.0x) |
| 371 | 0.015 | 0.014 | (0.94x) |
| 372 | 0.028 | 0.034 | (1.2x) |
| 373 | 0.020 | 0.025 | (1.3x) |
| 374 | 0.10 | 0.099 | (1.0x) |
| 375 | 0.022 | 0.014 | (0.65x) |
| 376 | 20 | 20 | (1.0x) |
| 377 | 10 | 20 | (2.0x) |
| 378 | 0.011 | 0.011 | (1.0x) |
| 379 | 0.035 | 0.048 | (1.4x) |
| 380 | 0.089 | 0.099 | (101x) |
| 381 | 1.1 | 7.3 | (5.9x) |
| 382 | 0.82 | 20 | (24x) |
| 383 | 0.49 | 0.38 | (0.77x) |
| 384 | 0.022 | 0.025 | (1.1x) |
| 385 | 0.024 | 0.022 | (0.93x) |
| 386 | 0.032 | 0.031 | (0.98x) |
| 387 | 0.058 | 0.053 | (0.91x) |
| 388 | 0.046 | 0.063 | (1.4x) |
| 389 | 0.0096 | 0.014 | (1.4x) |
| 390 | 0.096 | 0.011 | (1.1x) |
| 391 | 0.033 | 0.063 | (1.9x) |
| 392 | 0.013 | 0.015 | (1.1x) |
| 393 | 0.13 | 0.17 | (1.3x) |
| 394 | 0.12 | 0.16 | (1.3x) |
| 395 | 0.11 | 0.11 | (1.0x) |
| 396 | 0.12 | 0.15 | (1.3x) |
| 397 | 0.15 | 0.15 | (1.0x) |
| 398 | 0.14 | 0.18 | (1.3x) |
| 399 | 0.51 | 0.43 | (0.84x) |
| 400 | 0.039 | 0.051 | (1.3x) |
| 401 | 0.029 | 0.027 | (0.95x) |
| 402 | 0.039 | 0.046 | (1.2x) |
| 403 | 0.045 | 0.045 | (1.0x) |
| 404 | 0.010 | 0.0054 | (0.54x) |
| 405 | 0.018 | 0.016 | (0.91x) |
| 406 | 0.075 | 0.084 | (1.1x) |
| 407 | 0.0071 | 0.0057 | (0.80x) |
| 408 | 0.013 | 0.0091 | (0.68x) |
| 409 | 0.012 | 0.0059 | (0.48x) |
| 410 | 0.012 | 0.077 | (0.62x) |
| 411 | 0.048 | 0.043 | (0.88x) |
| 412 | 0.050 | 0.051 | (1.1x) |
| 413 | 0.056 | 0.050 | (0.90x) |
| 414 | 0.088 | 0.068 | (0.77x) |
| 415 | 0.016 | 0.014 | (0.91x) |
| 416 | 0.092 | 0.11 | (1.2x) |
| 417 | 0.057 | 0.052 | (0.93x) |
| 418 | 0.032 | 0.037 | (1.2x) |
| 419 | 0.36 | 0.42 | (1.2x) |
| 420 | 0.24 | 0.22 | (0.91x) |
| 421 | 0.015 | 0.011 | (0.72x) |
| 422 | 0.016 | 0.017 | (1.1x) |
| 423 | 0.023 | 0.023 | (1.0x) |
| 424 | 0.042 | 0.031 | (0.73x) |
| 425 | 0.032 | 0.022 | (0.68x) |
| 426 | 0.035 | 0.026 | (0.73x) |
| 427 | 0.021 | 0.016 | (0.76x) |
| 428 | 0.014 | 0.010 | (0.74x) |
| 429 | 0.026 | 0.020 | (0.76x) |
| 430 | 0.043 | 0.035 | (0.80x) |
| 431 | 0.048 | 0.042 | (0.87x) |
| 432 | 0.017 | 0.013 | (0.78x) |
| 433 | 0.094 | 0.12 | (1.3x) |
| 434 | 0.018 | 0.017 | (0.95x) |
| 435 | 0.019 | 0.019 | (1.0x) |
| 436 | 0.034 | 0.032 | (0.97x) |
| 437 | 0.062 | 0.90 | (0.97x) |
| 438 | 0.041 | 0.032 | (0.80x) |
| 439 | 0.012 | 0.0071 | (0.58x) |
| 440 | 0.14 | 0.11 | (0.79x) |
| 441 | 0.042 | 0.023 | (0.56x) |
| 442 | 0.096 | 0.064 | (0.67x) |
| 443 | 0.059 | 0.18 | (2.8x) |
| 444 | 0.17 | 0.18 | (1.1x) |
| 445 | 0.026 | 0.033 | (1.3x) |
| 446 | 0.026 | 0.029 | (1.3x) |
| 447 | 0.016 | 0.020 | (1.2x) |
| 448 | 0.014 | 0.016 | (1.1x) |
| 449 | 0.020 | 0.021 | (1.1x) |
| 450 | 0.015 | 0.014 | (0.95x) |
| 451 | 0.015 | 0.010 | (0.71x) |
| 452 | 0.012 | 0.0091 | (0.78x) |
| 453 | 0.014 | 0.018 | (1.4x) |
| 454 | 11 | 6.2 | (0.57x) |
| 455 | 4.4 | 3.1 | (0.70x) |
| 456 | 0.013 | 0.0076 | (0.56x) |
| 457 | 0.017 | 0.012 | (0.71x) |
| 458 | 0.023 | 0.015 | (0.66x) |
| 459 | 0.013 | 0.0087 | (0.70x) |
| 460 | 0.014 | 0.0084 | (0.58x) |
| 461 | 0.0099 | 0.0082 | (0.82x) |
| 462 | 0.0083 | 0.0079 | (0.95x) |
| 463 | 0.0060 | 0.0035 | (0.55x) |
| 464 | 0.012 | 0.012 | (1.0x) |
| 465 | 0.014 | 0.013 | (0.90x) |
| 466 | 0.0064 | 0.0061 | (0.96x) |
| 467 | 0.012 | 0.011 | (0.89x) |
| 468 | 0.015 | 0.015 | (1.0x) |
| 469 | 0.048 | 0.052 | (1.1x) |
| 470 | 0.012 | 0.0085 | (0.70x) |
| 471 | 0.0082 | 0.0068 | (0.83x) |
| 472 | 0.015 | 0.013 | (0.83x) |
| 473 | 0.015 | 0.017 | (1.2x) |
| 474 | 0.011 | 0.0086 | (0.79x) |
| 475 | 0.054 | 0.069 | (1.3x) |
| 476 | 0.047 | 0.039 | (0.83x) |
| 477 | 0.036 | 0.11 | (3.4x) |
| 478 | 0.082 | 0.16 | (2.0x) |
| 479 | 0.18 | 0.29 | (1.6x) |
| 480 | 0.059 | 0.18 | (2.8x) |
| 481 | 0.11 | 0.42 | (3.7x) |
| 482 | 0.21 | 0.41 | (1.9x) |
| 483 | 1.8 | 20 | (12x) |
| 484 | 0.23 | 1.2 | (4.8x) |
| 485 | 0.031 | 0.029 | (0.92x) |
| 486 | 0.077 | 0.093 | (1.2x) |
| 487 | 1.8 | 2.7 | (1.5x) |
| 488 | 1.9 | 12.5 | (6.4x) |
| 489 | 5.0 | 19.5 | (3.8x) |
| 490 | 3.3 | 3.5 | (1.0x) |
| 491 | 8.6 | 20 | (2.3x) |
| 492 | 0.057 | 0.060 | (1.0x) |
| 493 | 0.63 | 4.5 | (6.9x) |
| 494 | 0.013 | 0.012 | (0.94x) |

Cbl-b Lck HTRF data in Table 7 is measured according to Example 76 herein; C-cbl Lck HTRF data is measured according to Example 76 herein.

### Example 87: Cbl-b and c-Cbl SPR Assay

Affinity of binding to Cbl-b and c-Cbl for compounds described herein can be assessed by surface plasmon resonance (SPR) according to the following protocol. All experiments were recorded on a Biacore™ 8K or Biacore™ 8K+(Cytiva) with both surface preparation and experimental measurements performed at 20° C. in an assay buffer consisting of 50 mM HEPES, pH 7.5, 0.15 M NaCl, 0.001% (v/v) Tween® 20, 0.2 mM tris(2-carboxyethyl)phosphine, 0.025% (w/v) carboxymethylated dextran (average MW 10 kDa), 0.2% (w/v) PEG 3350, and 2% DMSO.

Human Cbl-b (residues 40-426) or c-Cbl (residues 47-435) were irreversibly captured to a Series S sensor chip SA (Cytiva 29104992) via an N-terminal avi-tag, biotinylated by co-expression in E. coli with BirA. A surface capture range of 1300-1500 RU of protein was used for both isoforms.

For SPR measurements, 6 concentrations with 2 fold serial dilution were measured with blanks flanking each series for double referencing. Initial concentrations between 20 and 0.5 μM were used depending on the anticipated affinity of the tested compound. SPR sensorgrams were recorded in multi-cycle kinetics format, with a contact time of 60 seconds and a flow rate of 40 μl/min, the dissociation time was varied between 120-1200 seconds aiming for 4-5 half-lives of the measured interaction.

Kinetic and affinity parameters were extracted from the multicycle kinetics data fitting to a 1:1 binding model using the Biacore™ Incyte evaluation software (Cytiva).

### Multi-Point Chaser SPR Assay Variation

For the purposes of this type of experiment the term “chaser compound” refers to a low affinity analogue of the compound under investigation which binds close to saturation at the used concentration and fully dissociates within 120 seconds. For the studies presented herein the chaser compound is Compound 254.

Affinity of binding to Cbl-b and c-Cbl for potent compounds described herein (Kd<10 nM) were assessed by surface plasmon resonance (SPR) using a “Chaser” assay format.

Chaser assay utilize a single cycle kinetics SPR experiment with a contact time of 120 seconds, a flow rate of 50 μl/min and a dissociation time of 450 seconds. Single cycle kinetics titration utilized an initial blank injection and 5 concentrations with 2 fold serial dilution with a maximum concentration of 500 nM, blanked to a preceding 6 point blank single cycle kinetics injection for double referencing.

In the case of potent compounds, the protein-compound half-life cannot be accurately measured using routine fitting of the single cycle titration data. The kd is measured independently by determining the percentage unoccupied compound binding site over time by measuring the binding of a chaser compound measured by SPR.

Chaser binding was measured by a multicycle kinetics SPR experiment using a contact time of 20 seconds, a flow rate of 30 μl/min, and a dissociation time of 120 seconds. 7 injections of a single chaser concentration of 15 pM with a preceding blank injection, were recorded spaced out between 674 and 30,263 seconds after the last single cycle kinetics titration injection. The % compound bound at a given time was determined by comparison to a single injection of chaser preceding the single cycle kinetics titration defined below.

$$\% Compound Bound = {( 1 - ( RUT / RUT 0 ) )}^{*} 100$$

Where RUT is the observed SPR signal for chaser injection at time T, and RUT0 is the observed SPR signal for chaser injected prior to the titration of compound under investigation.

The % compound bound is plotted against time in seconds and fit to a single exponential, where the exponent represents the kd of the compound-protein complex. The single cycle kinetics experiment of the compound is then fit with a fixed kd determined using the chaser.

SPR and the LCK biochemical assay are orthogonal assays: SPR is a protein binding assay while the LCK assay is an enzyme activity assay. SPR measures compound binding affinity to CBL-B/C-CBL, whereas LCK assay measures compound inhibition of CBL-B/C-CBL ubiquitin transfer activity.

### Example 88: LCMS Methods

The following LCMS methods were used to obtain mass spectrometric data of compounds described elsewhere herein.

### LC-MS Method A: 5-95AB_1.5 Min_220 and 254_Shimadzu

| Column | MERCK, RP-18e 25-3 mm | |
|---|---|---|
| Mobile Phase | A: water (4 L) + TFA (1.5 mL) | |
| | B: acetonitrile (4 L) + TFA (0.75 mL) | |
| | TIME (min) | B % |
| | 0 | 5 |
| | 0.7 | 95 |
| | 1.1 | 95 |
| | 1.11 | 5 |
| | 1.5 | 5 |

| | |
|---|---|
| Flow Rate | 1.5 mL/min |
| wavelength | UV 220 nm, 254 nm |
| Oven Temp | 50° C. |
| Instrument | SHIMADZU LC20-MS2010 |
| MS ionization | ESI |

### Description

Mobile Phase: 1.5 mL/4 L TFA in water (solvent A) and 0.75 mL/4 L TFA in acetonitrile (solvent B), using the elution gradient 5%-95% (solvent B) over 0.7 minutes and holding at 95% for 0.4 minutes at a flow rate of 1.5 ml/min;
Column: MERCK, RP-18e 25-3 mm;
Wavelength: UV 220 nm, 254 nm;
Column temperature: 50° C.; MS ionization: ESI

### LC-MS Method B: 10-80 CD_3MIN_220 and 254_Shimadzu

| Column | Xbridge Shield RP-18, 5 um, 2.1*50 mm | |
|---|---|---|
| Mobile Phase | A: water (4 L) + NH₃•H₂O (0.8 mL) | |
| | B: acetonitrile | |
| | TIME (min) | B % |
| | 0 | 10 |
| | 2 | 80 |
| | 2.48 | 80 |
| | 2.49 | 10 |
| | 3 | 10 |

| | |
|---|---|
| Flow Rate | 1 mL/min |
| wavelength | UV 220 nm and 254 nm |
| Oven Temp | 50° C. |
| MS ionization | ESI |
| Instrument | SHIMADZU LC20-MS2020 |

### Description

Mobile Phase: 0.8 mL/4 L NH₃·H₂O in water (solvent A) and acetonitrile (solvent B), using the elution gradient 10%-80% (solvent B) over 2 minutes and holding at 80% for 0.48 minutes at a flow rate of 1 ml/min;
Column: XBridge C18 Sum, 2.1*50 mm;
Wavelength: UV 220 nm and 254 nm;
Column temperature: 50° C.; MS ionization: ESI
   LC-MS Method C: 5-95AB_1 min_220 and 254_Agilent

| Column | Agilent Poroshell 120 EC-C18 2.7 um 3.0*30 mm | |
|---|---|---|
| Mobile Phase | A: water (4 L) + TFA (1.5 mL) | |
| | B: acetonitrile (4 L) + TFA (0.75 mL) | |
| | TIME (min) | B % |
| | 0 | 5 |
| | 0.4 | 95 |
| | 0.7 | 95 |
| | 0.71 | 5 |
| | 1 | 5 |

| | |
|---|---|
| Flow Rate | 2.0 mL/min |
| wavelength | UV 220 nm and 254 nm |
| Oven Temp | 50° C. |
| Instrument | Agilent Prime-6125B |
| MS ionization | ESI |

### Description

Mobile Phase: 1.5 mLa4 LTFA in water (solvent A) and 0.75 mL4 LTFA in acetonitrile (solvent B), using the elution gradient 5%-95% (solvent B) over 0.4 minutes and holding at 95% for 0.3 minutes at a flow rate of 2.0 ml/min;
Column: Agilent Poroshell 120 EC-C18 2.7 um 3.0*30 mm;
Wavelength: UV 220 nm and 254 nm;
Column temperature: 50° C.;
MS ionization: ESI

### LC-MS Method D: 5-95AB_220 and 254_Agilent

| Column | MERCK, RP-18e 25-2 mm | |
|---|---|---|
| Mobile Phase | A: water (4 L) + TFA (1.5 mL) | |
| | B: acetonitrile (4 L) + TFA (0.75 mL) | |
| | TIME (min) | B % |
| | 0 | 5 |
| | 0.7 | 95 |
| | 1.1 | 95 |
| | 1.11 | 5 |
| | 1.5 | 5 |

| | |
|---|---|
| Flow Rate | 1.5 mL/min |
| wavelength | UV 220 nm and 254 nm |
| Oven Temp | 50° C. |
| Instrument | Agilent LC1200-MS6110 |
| MS ionization | ESI |

### Description

Mobile Phase: 1.5 mL/4 L TFA in water (solvent A) and 0.75 mL/4 LTFA in acetonitrile (solvent B), using the elution gradient 5%-95% (solvent B) over 0.7 minutes and holding at 95% for 0.4 minutes at a flow rate of 1.5 ml/min;
Column: MERCK, RP-18e 25-2 mm;
Wavelength: UV 220 nm and 254 nm;
Column temperature: 50° C.;
MS ionization: ESI.

### LC-MS Method E: 10-80AB_7 min 220 and 254_Shimadzu

| Column | Xtimate C18 2.1*30 mm, 3 μm | |
|---|---|---|
| Mobile Phase | A: water (4 L) + TFA (1.5 mL) | |
| | B: acetonitrile (4 L) + TFA (0.75 mL) | |
| | TIME (min) | B % |
| | 0 | 10 |
| | 6 | 80 |
| | 6.5 | 80 |
| | 6.51 | 10 |
| | 7 | 10 |

| | |
|---|---|
| Flow Rate | 0.8 mL/min |
| wavelength | UV 220 nm, 254 nm |
| Oven Temp | 50° C. |
| Instrument | SHIMADZU LC20-MS2010 |
| MS ionization | ESI |

### Description

Mobile Phase: 1.5 mL/4 L, TFA in water (solvent A) and 0.75 mL/4 L TFA in acetonitrile (solvent B), using the elution gradient 10%-80% (solvent B) over 6 minutes and holding at 80% for 0.5 minutes at a flow rate of 0.8 ml/min;
Column: Xtimate C18 2.1*30 mm, 3 μm;
Wavelength: UV 220 nm, 254 nm;
Column temperature: 50° C.;
MS ionization: ESI

### Example 89: Comparative Activity and Physicochemical Data

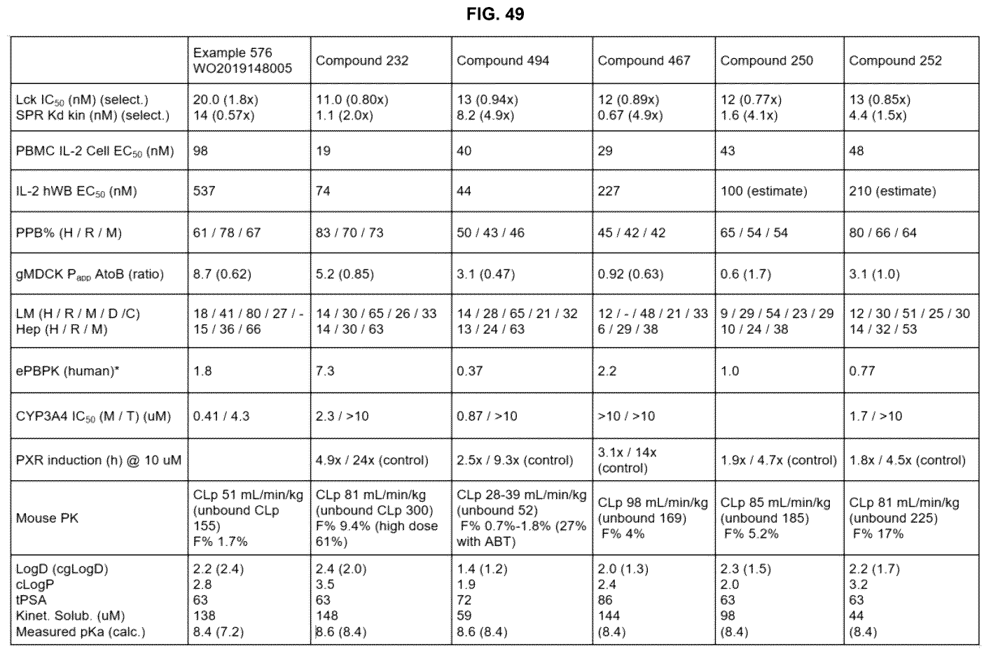

FIG. 49 shows a table of comparative potency and selectivity data for various compounds herein, as compared to a compound known in the art. In one aspect, the compounds of the present disclosure have greater human liver microsome stability (LM(H)) than a compound in the art.

In the table of FIG. 49, the ePBPK (human) data is a predicted human unbound conc.@ 12 hr over PBMC cell EC₅₀ at 5 g dose.

In the CYP3A4 measurements, potency is less desirable. The compounds of the present examples are less potent than the reference compound in the art.

All references cited herein are incorporated by reference in their entireties.

The foregoing description and the associated drawings is intended to illustrate various aspects of the instant technology. It is not intended that the examples presented herein limit the scope of the appended claims. The invention now being fully described, it will be apparent to one of ordinary skill in the art that many changes and modifications can be made thereto without departing from the spirit or scope of the appended claims.

1.-48. (canceled)

49. A compound of formula (I-C), 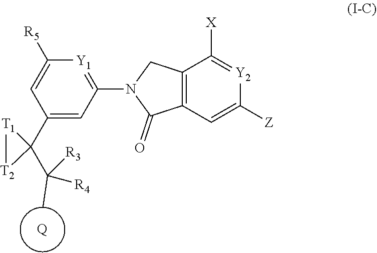
wherein:
Q is a 5-membered heteroaryl, optionally substituted by one or more alkyl, aryl, cycloalkyl, or haloalkyl groups;
Y₁ and Y₂ are independently CH, CF, or N;
T₁ and T₂ are each independently a group selected from: [—C(R₁)(R₂)—]ₙ, —O—, —C(R₁)(R₂)—O—, >C═O, —C(R₁)(R₂)—C(═O)—, —C(R₁)(R₂)—S(═O)₂—, and >S(═O)₂, with the proviso that T₁ and T₂ together are not —CH₂—O—, —O—O—, or —O—C(═O)—O—;
wherein n=0, 1 or 2, and each of R₁ and R₂ is independently selected from: H, halo, alkyl, alkenyl, cyano, hydroxyl, alkoxy, cycloalkyl, hydroxyalkyl, and haloalkyl, with the proviso that, when T₁ and T₂ together are —CH₂C(R₁)(R₂)CH₂—, neither of R₁ and R₂ is cyano;
or R₁ and R₂ together with the carbon atom to which they are both bonded form a cycloalkyl or heterocyclyl ring;
R₃, and R₄ are independently selected from: H, and halo, alkyl, CN, OH, alkoxy, and haloalkyl;
R₅ is selected from: H, halo, CN, or L₁ₐ-R₁₀, wherein Lia is —C(L_{1b}R₁₁)(R₁₂)—, —N(L_{1b}R₁₁)—, —C(═O)N(R₁₁)—, O, S, carbonyl, or a bond, wherein:
L_{1b} is alkylene or a bond; and
R₁₀, R₁₁ and R₁₂ are independently H, alkyl, alkoxyalkyl, aminoalkyl, cycloalkyl, bridged bicyclyl, fused bicyclyl, spirocyclyl, alkenyl, cycloalkenyl, alkynyl, amidoalkyl, aryl, heteroaryl, or heterocyclyl, and R₁₀, R₁₁ and R₁₂ are each optionally substituted by one or more groups selected from: halo, CN, amino, alkylamino, alkyl carbonyl, OH, oxo, alkoxy, alkyl, cycloalkyl, haloalkyl, alkoxyalkyl, sulfonamidyl, alkyl sulfonamidyl, heterocyclyl, aryl, and heteroaryl;
X is halo, haloalkyl, or cycloalkyl; and
Z is —L₂NR₇R₈;
wherein:
L₂ is —C(H)R₆ₐ;
R₆ₐ=H, alkyl, cycloalkyl, or haloalkyl; and
R₇ is selected from alkyl, cycloalkyl, spirocyclyl, bridged bicyclyl, hydroxyalkyl, heterocyclyl, and haloalkyl,
wherein, if R₇ is alkyl, cycloalkyl or heterocyclyl, said alkyl,
cycloalkyl or heterocyclyl group is optionally substituted with one or more groups selected from: sulfonyl, halo, hydroxyl, alkoxy, alkyl, cycloalkyl, alkoxyalkyl, alkenyl, heterocyclyl, hydroxyalkyl, cyano, carboxylalkyl, and haloalkyl; and
R₈ is H,
or an enantiomer, diastereomer or a pharmaceutically acceptable salt or solvate thereof.

50. The compound of claim 49, wherein T₁ and T₂ together are —CH₂C(R₁)R₂)CH₂—, and each of R₁ and R₂ is independently selected from: H, halo, alkyl, alkenyl, hydroxyl, alkoxy, cycloalkyl, hydroxyalkyl, and haloalkyl.

51. (canceled)

52. The compound of claim 49, wherein Y₁ and Y₂ are CH.

53. The compound of claim 49, wherein X is CF₃.

54.-57. (canceled)

58. The compound of claim 49, wherein Z is —CH₂NHR₇, wherein R₇ is cycloalkyl substituted by an alkyl group.

59. The compound of claim 58, wherein R₇ is 1-methyl-cyclobut-1-yl.

60.-86. (canceled)

87. A compound of formula (I-G), 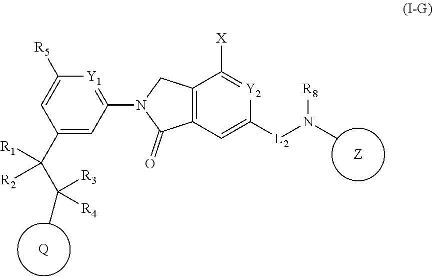
wherein:
Q is a 5-membered heteroaryl group, optionally substituted by one or more aryl, alkyl, cycloalkyl, or haloalkyl groups;
Y₁ and Y₂ are independently CH, CF, or N;
R₁, R₂, R₃, R₄ are independently selected from: H, halogen, alkyl, cycloalkyl, CN, OH, alkoxy, and haloalkyl; or
R₁ and R₂, together with the carbon atom to which they are both bonded form a 3-5 membered cycloalkyl or heterocyclyl ring, optionally substituted with one or more groups independently selected from: halogen, OH, sulfonyl, alkoxy, alkyl, cycloalkyl, hydroxyalkyl, or haloalkyl;
R₅ is selected from: H, halo, CN, or L₁ₐ-R₆, wherein L₁ₐ is —C(L_{1b} R₇)(R₈)—, —N(L_{1b} R₇)—, —C(═O)N(L_{1b}R₇)—, O, S, carbonyl, or a bond, wherein:
L_{1b} is alkylene or a bond; and
R₆ and R₇ are independently H, alkyl, alkoxyalkyl, aminoalkyl, cycloalkyl, bridged bicyclyl, fused bicyclyl, spirocyclyl, alkenyl, cycloalkenyl, alkynyl, aryl, amidoalkyl, heteroaryl, or heterocyclyl, and R₆, R₇ and R₈ are optionally substituted by one or more groups selected from: halo, CN, amino, alkylamino, alkyl carbonyl, OH, oxo, alkoxy, alkyl, cycloalkyl, haloalkyl, alkoxyalkyl, sulfonamidyl, alkyl sulfonamidyl, heterocyclyl, aryl, and heteroaryl,
R₈ is H;
X is halo, haloalkyl, or cycloalkyl;
L₂ is —C(H)R₉—, wherein R₉=H, alkyl, cycloalkyl, or haloalkyl; and
Z is a 3-10 membered ring system, optionally substituted by one or more substituents independently selected from: H, sulfonyl, halo, hydroxyl, alkoxy, alkyl, cycloalkyl, heterocyclyl, alkoxyalkyl, alkenyl, hydroxyalkyl, oxo, carboxylalkyl, and haloalkyl;
or an enantiomer, diastereomer or a pharmaceutically acceptable salt or solvate thereof.

88. A compound of claim 87, wherein Z is a monocyclic ring substituted by an alkyl group.

89.-93. (canceled)

94. The compound of claim 87, wherein R₁ and R₂, together with the carbon atom to which they are both bonded form a ring selected from cyclopropyl, cyclobutyl and oxetane.

95.-97. (canceled)

98. The compound of claim 87, wherein R₅ is H.

99. The compound of claim 87, wherein Q is 4-methyl-2H-1,2,3-triazol-2-yl, 4-methyl-1H-1,2,3-triazol-1-yl, 5-methyl-1H-1,2,3-triazol-1-yl, 5-methyl-1H-pyrazol-1-yl, 3-methyl-1H-pyrazol-1-yl, 1-methyl-1H-imidazol-2-yl, or 4-methyl-4H-1,2,4-triazol-3-yl.

100. A pharmaceutical composition comprising a compound, or a pharmaceutically acceptable salt thereof, according to claim 87, and one or more pharmaceutically acceptable excipients.

101. A method of treating a cancer, the method comprising administering to a subject in need thereof, a therapeutic amount of a compound according to claim 87.

102. A pharmaceutical composition comprising a compound, or a pharmaceutically acceptable salt thereof, according to claim 49, and one or more pharmaceutically acceptable excipients.

103. A method of treating a cancer, the method comprising administering to a subject in need thereof, a therapeutic amount of a compound according to claim 49.

104. A compound of claim 49, selected from the group consisting of:
(2-(3-(3,3-difluoro-1-((4-methyl-4H-1,2,4-triazol-3-yl)methyl)cyclobutyl)phenyl)-6-(((1-methylcyclobutyl)amino)methyl)-4-(trifluoromethyl)isoindolin-1-one,
2-(3-(1-(difluoro(4-methyl-4H-1,2,4-triazol-3-yl)methyl)cyclobutyl)phenyl)-6-(((1-methylcyclobutyl)amino)methyl)-4-(trifluoromethyl)isoindolin-1-one,
2-(6-((cyclopropylmethyl)amino)-4-(1-((4-methyl-4H-1,2,4-triazol-3-yl)methyl)cyclobutyl)pyridin-2-yl)-6-(((1-methylcyclobutyl)amino)methyl)-4-(trifluoromethyl)-isoindolin-1-one,
2-(6-benzyl-4-(1-((4-methyl-4H-1,2,4-triazol-3-yl)methyl)cyclobutyl)pyridin-2-yl)-6-(((1-methylcyclobutyl)amino)methyl)-4-(trifluoromethyl)isoindolin-1-one,
2-(3-(3-((4-methyl-4H-1,2,4-triazol-3-yl)methyl)-1,1-dioxidothietan-3-yl)phenyl)-6-(((1-methyl-cyclobutyl)amino)methyl)-4-(trifluoromethyl)isoindolin-1-one,
2-(3-(3-((5-methyl-1H-1,2,3-triazol-1-yl)methyl)oxetan-3-yl)phenyl)-6-(((1-methylcyclobutyl)amino)methyl)-4-(trifluoromethyl)isoindolin-1-one,
2-(3-(3,3-difluoro-1-((4-methyl-4H-1,2,4-triazol-3-yl)methyl)cyclobutyl)phenyl)-5-fluoro-6-(((1-methylcyclobutyl)amino)methyl)-4-(trifluoromethyl)isoindolin-1-one,
(R)-2-(3-(3,3-difluoro-1-((4-methyl-4H-1,2,4-triazol-3-yl)methyl)cyclobutyl)phenyl)-6-(1-((1-methylcyclobutyl)amino)ethyl)-4-(trifluoromethyl)isoindolin-1-one,
(S)-2-(3-(3,3-difluoro-1-((4-methyl-4H-1,2,4-triazol-3-yl)methyl)cyclobutyl)phenyl)-6-(1-((1-methylcyclobutyl)amino)ethyl)-4-(trifluoromethyl)isoindolin-1-one,
2-(3-(3-((R)-fluoro(4-methyl-4H-1,2,4-triazol-3-yl)methyl)oxetan-3-yl)phenyl)-6-((R)-1-((1-methylcyclobutyl)amino)ethyl)-4-(trifluoromethyl)isoindolin-1-one,
2-(3-(3-((R)-fluoro(4-methyl-4H-1,2,4-triazol-3-yl)methyl)oxetan-3-yl)phenyl)-6-((S)-1-((1-methylcyclobutyl)amino)ethyl)-4-(trifluoromethyl)isoindolin-1-one,
(R)-4-(difluoromethyl)-2-(3-(3-(fluoro(4-methyl-4H-1,2,4-triazol-3-yl)methyl)oxetan-3-yl)phenyl)-6-(((1-methylcyclobutyl)amino)methyl)isoindolin-1-one,
2-(3-(3,3-difluoro-1-((4-methyl-4H-1,2,4-triazol-3-yl)methyl)cyclobutyl)phenyl)-4-(difluoromethyl)-6-(((1-methylcyclobutyl)amino)methyl)isoindolin-1-one,
2-(4-(1-((4-methyl-4H-1,2,4-triazol-3-yl)methyl)cyclobutyl)-6-(methylthio)pyridin-2-yl)-6-(((1-methylcyclobutyl)amino)methyl)-4-(trifluoromethyl)isoindolin-1-one, and
4-chloro-2-(3-(3,3-difluoro-1-((4-methyl-4H-1,2,4-triazol-3-yl)methyl)cyclobutyl)-phenyl)-6-(((1-methylcyclobutyl)amino)methyl)isoindolin-1-one,
or a pharmaceutically acceptable salt thereof.

105. A compound of claim 49, selected from the group consisting of:
(R)-2-(3-(3-(fluoro(4-methyl-4H-1,2,4-triazol-3-yl)methyl)oxetan-3-yl)phenyl)-6-((methylamino)methyl)-4-(trifluoromethyl)isoindolin-1-one,
(R)-6-((ethylamino)methyl)-2-(3-(3-(fluoro(4-methyl-4H-1,2,4-triazol-3-yl)methyl)oxetan-3-yl)phenyl)-4-(trifluoromethyl)isoindolin-1-one,
(R)-2-(3-(3-(fluoro(4-methyl-4H-1,2,4-triazol-3-yl)methyl)oxetan-3-yl)phenyl)-6-((isopropylamino)methyl)-4-(trifluoromethyl)isoindolin-1-one,
(R)-6-((Cyclobutylamino)methyl)-2-(3-(3-(fluoro(4-methyl-4H-1,2,4-triazol-3-yl)methyl)oxetan-3-yl)phenyl)-4-(trifluoromethyl)isoindolin-1-one,
(R)-2-(3-(3-(fluoro(4-methyl-4H-1,2,4-triazol-3-yl)methyl)oxetan-3-yl)phenyl)-6-((propylamino)methyl)-4-(trifluoromethyl)isoindolin-1-one,
(R)-2-(3-(3-(fluoro(4-methyl-4H-1,2,4-triazol-3-yl)methyl)oxetan-3-yl)phenyl)-6-(((1-methylcyclopropyl)amino)methyl)-4-(trifluoromethyl)isoindolin-1-one,
(R)-2-(3-(3-(fluoro(4-methyl-4H-1,2,4-triazol-3-yl)methyl)oxetan-3-yl)phenyl)-6-(((1-methylcyclobutyl)amino)methyl)-4-(trifluoromethyl)isoindolin-1-one, and
(R)-2-(3-(3-(fluoro(4-methyl-4H-1,2,4-triazol-3-yl)methyl)oxetan-3-yl)phenyl)-6-(((3-fluoropropyl)amino)methyl)-4-(trifluoromethyl)isoindolin-1-one,
or a pharmaceutically acceptable salt thereof.

106. A compound of claim 49, selected from the group consisting of:
2-(3-(3,3-difluoro-1-((4-methyl-4H-1,2,4-triazol-3-yl)methyl)cyclobutyl)phenyl)-5-fluoro-6-(((1-methylcyclobutyl)amino)methyl)-4-(trifluoromethyl)isoindolin-1-one,
2-(6-(cyclopentylamino)-4-(3-((4-methyl-4H-1,2,4-triazol-3-yl)methyl)oxetan-3-yl)pyridin-2-yl)-6-((methylamino)methyl)-4-(trifluoromethyl)isoindolin-1-one,
4-chloro-2-(3-(3,3-difluoro-1-((4-methyl-4H-1,2,4-triazol-3-yl)methyl)cyclobutyl)phenyl)-6-(((1-methylcyclobutyl)amino)methyl)isoindolin-1-one,
6-((tert-butylamino)methyl)-2-(3-(3,3-difluoro-1-((4-methyl-4H-1,2,4-triazol-3-yl)methyl)cyclobutyl)phenyl)-4-(trifluoromethyl)isoindolin-1-one,
2-(6-cyclopropyl-4-(1-((4-methyl-4H-1,2,4-triazol-3-yl)methyl)cyclobutyl)pyridin-2-yl)-6-(((1-methylcyclobutyl)amino)methyl)-4-(trifluoromethyl)isoindolin-1-one,
2-(6-ethoxy-4-(1-((4-methyl-4H-1,2,4-triazol-3-yl)methyl)cyclobutyl)pyridin-2-yl)-6-(((1-methylcyclobutyl)amino)methyl)-4-(trifluoromethyl)isoindolin-1-one,
2-(6-(ethylthio)-4-(1-((4-methyl-4H-1,2,4-triazol-3-yl)methyl)cyclobutyl)pyridin-2-yl)-6-(((1-methylcyclobutyl)amino)methyl)-4-(trifluoromethyl)isoindolin-1-one,
2-(3-(3-(difluoro(4-methyl-4H-1,2,4-triazol-3-yl)methyl)oxetan-3-yl)phenyl)-6-(((1-methylcyclobutyl)amino)methyl)-4-(trifluoromethyl)isoindolin-1-one,
2-(3-(3,3-difluoro-1-((4-methyl-4H-1,2,4-triazol-3-yl)methyl)cyclobutyl)phenyl)-4-(difluoromethyl)-6-(((1-methylcyclobutyl)amino)methyl)isoindolin-1-one,
(R)-4-(difluoromethyl)-2-(3-(3-(fluoro(4-methyl-4H-1,2,4-triazol-3-yl)methyl)oxetan-3-yl)phenyl)-6-(((1-methylcyclobutyl)amino)methyl)isoindolin-1-one,
2-(6-(ethylamino)-4-(3-((4-methyl-4H-1,2,4-triazol-3-yl)methyl)oxetan-3-yl)pyridin-2-yl)-6-(((1-methylcyclobutyl)amino)methyl)-4-(trifluoromethyl)isoindolin-1-one,
2-(6-(cyclopentyloxy)-4-(3-((4-methyl-4H-1,2,4-triazol-3-yl)methyl)oxetan-3-yl)pyridin-2-yl)-6-(((1-methylcyclobutyl)amino)methyl)-4-(trifluoromethyl)isoindolin-1-one,
2-(6-(cyclopentylamino)-4-(1-((4-methyl-4H-1,2,4-triazol-3-yl)methyl)cyclobutyl)pyridin-2-yl)-6-(((1-methylcyclobutyl)amino)methyl)-4-(trifluoromethyl)isoindolin-1-one,
2-(6-(cyclopentylamino)-4-(3-((4-methyl-4H-1,2,4-triazol-3-yl)methyl)oxetan-3-yl)pyridin-2-yl)-6-(((1-methylcyclobutyl)amino)methyl)-4-(trifluoromethyl)isoindolin-1-one,
2-(6-cyclopropyl-4-(3-((4-methyl-4H-1,2,4-triazol-3-yl)methyl)oxetan-3-yl)pyridin-2-yl)-6-(((1-methylcyclobutyl)amino)methyl)-4-(trifluoromethyl)isoindolin-1-one,
2-(3-(3-((4-methyl-4H-1,2,4-triazol-3-yl)methyl)oxetan-3-yl)phenyl)-6-(((1-methylcyclobutyl)amino)methyl)-4-(trifluoromethyl)isoindolin-1-one,
2-(6-((1-(2-methoxyethyl)piperidin-4-yl)amino)-4-(1-((4-methyl-4H-1,2,4-triazol-3-yl)methyl)cyclobutyl)pyridin-2-yl)-6-(((1-methylcyclobutyl)amino)methyl)-4-(trifluoromethyl)isoindolin-1-one,
2-(4-(1-((4-methyl-4H-1,2,4-triazol-3-yl)methyl)cyclobutyl)-6-((3-morpholinopropyl)amino)pyridin-2-yl)-6-(((1-methylcyclobutyl)amino)methyl)-4-(trifluoromethyl)isoindolin-1-one,
2-(6-ethoxy-4-(3-((4-methyl-4H-1,2,4-triazol-3-yl)methyl)oxetan-3-yl)pyridin-2-yl)-6-(((1-methylcyclobutyl)amino)methyl)-4-(trifluoromethyl)isoindolin-1-one,
4-(difluoromethyl)-2-(3-(3-((5-methyl-1H-1,2,3-triazol-1-yl)methyl)oxetan-3-yl)phenyl)-6-(((1-methylcyclobutyl)amino)methyl)isoindolin-1-one,
2-(6-(((1s,4s)-4-aminocyclohexyl)amino)-4-(1-((4-methyl-4H-1,2,4-triazol-3-yl)methyl)cyclobutyl)pyridin-2-yl)-6-(((1-methylcyclobutyl)amino)methyl)-4-(trifluoromethyl)isoindolin-1-one,
N-ethyl-2-((4-(1-((4-methyl-4H-1,2,4-triazol-3-yl)methyl)cyclobutyl)-6-(6-(((1-methylcyclobutyl)amino)methyl)-1-oxo-4-(trifluoromethyl)isoindolin-2-yl)pyridin-2-yl)amino)acetamide,
3-((4-(1-((4-methyl-4H-1,2,4-triazol-3-yl)methyl)cyclobutyl)-6-(6-(((1-methylcyclobutyl)amino)methyl)-1-oxo-4-(trifluoromethyl)isoindolin-2-yl)pyridin-2-yl)amino)propanenitrile,
2-(4-(1-((4-methyl-4H-1,2,4-triazol-3-yl)methyl)cyclobutyl)-6-((2-(4-methylpiperidin-1-yl)ethyl)amino)pyridin-2-yl)-6-(((1-methylcyclobutyl)amino)methyl)-4-(trifluoromethyl)isoindolin-1-one,
2-(4-(1-((4-methyl-4H-1,2,4-triazol-3-yl)methyl)cyclobutyl)-6-(methylamino)pyridin-2-yl)-6-(((1-methylcyclobutyl)amino)methyl)-4-(trifluoromethyl)isoindolin-1-one,
N,N-dimethyl-2-((4-(1-((4-methyl-4H-1,2,4-triazol-3-yl)methyl)cyclobutyl)-6-(6-(((1-methylcyclobutyl)amino)methyl)-1-oxo-4-(trifluoromethyl)isoindolin-2-yl)pyridin-2-yl)amino)acetamide,
N-methyl-2-((4-(1-((4-methyl-4H-1,2,4-triazol-3-yl)methyl)cyclobutyl)-6-(6-(((1-methylcyclobutyl)amino)methyl)-1-oxo-4-(trifluoromethyl)isoindolin-2-yl)pyridin-2-yl)amino)ethanesulfonamide,
2-(6-(((1-methyl-1H-pyrazol-4-yl)methyl)amino)-4-(1-((4-methyl-4H-1,2,4-triazol-3-yl)methyl)cyclobutyl)pyridin-2-yl)-6-(((1-methylcyclobutyl)amino)methyl)-4-(trifluoromethyl)isoindolin-1-one,
N-(4-((4-(1-((4-methyl-4H-1,2,4-triazol-3-yl)methyl)cyclobutyl)-6-(6-(((1-methylcyclobutyl)amino)methyl)-1-oxo-4-(trifluoromethyl)isoindolin-2-yl)pyridin-2-yl)amino)butyl)acetamide,
2-(6-(((1,1-dioxidotetrahydro-2H-thiopyran-4-yl)methyl)amino)-4-(1-((4-methyl-4H-1,2,4-triazol-3-yl)methyl)cyclobutyl)pyridin-2-yl)-6-(((1-methylcyclobutyl)amino)methyl)-4-(trifluoromethyl)isoindolin-1-one,
2-(6-((2-(1H-indol-3-yl)ethyl)amino)-4-(1-((4-methyl-4H-1,2,4-triazol-3-yl)methyl)cyclobutyl)pyridin-2-yl)-6-(((1-methylcyclobutyl)amino)methyl)-4-(trifluoromethyl)isoindolin-1-one,
2-(6-((2,2-difluoro-3-hydroxypropyl)amino)-4-(1-((4-methyl-4H-1,2,4-triazol-3-yl)methyl)cyclobutyl)pyridin-2-yl)-6-(((1-methylcyclobutyl)amino)methyl)-4-(trifluoromethyl)isoindolin-1-one,
2-(6-((3-((dimethylamino)methyl)benzyl)amino)-4-(1-((4-methyl-4H-1,2,4-triazol-3-yl)methyl)cyclobutyl)pyridin-2-yl)-6-(((1-methylcyclobutyl)amino)methyl)-4-(trifluoromethyl)isoindolin-1-one,
6-(((1-cyclopropylethyl)amino)methyl)-2-(3-(3,3-difluoro-1-((4-methyl-4H-1,2,4-triazol-3-yl)methyl)cyclobutyl)phenyl)-4-(trifluoromethyl)isoindolin-1-one,
2-(6-(((1-methyl-1H-imidazol-4-yl)methyl)amino)-4-(1-((4-methyl-4H-1,2,4-triazol-3-yl)methyl)cyclobutyl)pyridin-2-yl)-6-(((1-methylcyclobutyl)amino)methyl)-4-(trifluoromethyl)isoindolin-1-one,
2-(4-(1-((4-methyl-4H-1,2,4-triazol-3-yl)methyl)cyclobutyl)-6-((2-(pyridin-4-yl)ethyl)amino)pyridin-2-yl)-6-(((1-methylcyclobutyl)amino)methyl)-4-(trifluoromethyl)isoindolin-1-one,
2-(6-(3,3-dimethylbutyl)-4-(1-((4-methyl-4H-1,2,4-triazol-3-yl)methyl)cyclobutyl)pyridin-2-yl)-6-(((1-methylcyclobutyl)amino)methyl)-4-(trifluoromethyl)isoindolin-1-one,
2-(4-(1-((4-methyl-4H-1,2,4-triazol-3-yl)methyl)cyclobutyl)-6-(methylthio)pyridin-2-yl)-6-(((1-methylcyclobutyl)amino)methyl)-4-(trifluoromethyl)isoindolin-1-one,
2-(4-(1-((4-methyl-4H-1,2,4-triazol-3-yl)methyl)cyclobutyl)-6-phenylpyridin-2-yl)-6-(((1-methylcyclobutyl)amino)methyl)-4-(trifluoromethyl)isoindolin-1-one,
2-(4-(1-((4-methyl-4H-1,2,4-triazol-3-yl)methyl)cyclobutyl)-6-phenethylpyridin-2-yl)-6-(((1-methylcyclobutyl)amino)methyl)-4-(trifluoromethyl)isoindolin-1-one,
2-(4-(1-((4-methyl-4H-1,2,4-triazol-3-yl)methyl)cyclobutyl)pyridin-2-yl)-6-(((1-methylcyclobutyl)amino)methyl)-4-(trifluoromethyl)isoindolin-1-one,
2-(6-(1-methyl-1H-pyrazol-4-yl)-4-(1-((4-methyl-4H-1,2,4-triazol-3-yl)methyl)cyclobutyl)pyridin-2-yl)-6-(((1-methylcyclobutyl)amino)methyl)-4-(trifluoromethyl)isoindolin-1-one,
2-(3-(1-(difluoro(4-methyl-4H-1,2,4-triazol-3-yl)methyl)cyclobutyl)phenyl)-6-(((1-methylcyclobutyl)amino)methyl)-4-(trifluoromethyl)isoindolin-1-one,
2-(6-(((1-acetylpiperidin-4-yl)methyl)amino)-4-(1-((4-methyl-4H-1,2,4-triazol-3-yl)methyl)cyclobutyl)pyridin-2-yl)-6-(((1-methylcyclobutyl)amino)methyl)-4-(trifluoromethyl)isoindolin-1-one,
2-(3-(3-((4-methyl-4H-1,2,4-triazol-3-yl)methyl)oxetan-3-yl)phenyl)-6-(((1-methylcyclobutyl)amino)methyl)-4-(trifluoromethyl)isoindolin-1-one,
2-(3-(3,3-difluoro-1-((4-methyl-4H-1,2,4-triazol-3-yl)methyl)cyclobutyl)phenyl)-6-(((3-methyloxetan-3-yl)amino)methyl)-4-(trifluoromethyl)isoindolin-1-one,
6-(((cyclopropylmethyl)amino)methyl)-2-(3-(3,3-difluoro-1-((4-methyl-4H-1,2,4-triazol-3-yl)methyl)cyclobutyl)phenyl)-4-(trifluoromethyl)isoindolin-1-one,
2-(4-(1-((4-methyl-4H-1,2,4-triazol-3-yl)methyl)cyclobutyl)-6-(((tetrahydro-2H-pyran-4-yl)methyl)amino)pyridin-2-yl)-6-(((1-methylcyclobutyl)amino)methyl)-4-(trifluoromethyl)isoindolin-1-one,
2-(6-((2-(2-methoxyethoxy)ethyl)amino)-4-(1-((4-methyl-4H-1,2,4-triazol-3-yl)methyl)cyclobutyl)pyridin-2-yl)-6-(((1-methylcyclobutyl)amino)methyl)-4-(trifluoromethyl)isoindolin-1-one,
2-(6-((2-(dimethylamino)ethyl)amino)-4-(1-((4-methyl-4H-1,2,4-triazol-3-yl)methyl)cyclobutyl)pyridin-2-yl)-6-(((1-methylcyclobutyl)amino)methyl)-4-(trifluoromethyl)isoindolin-1-one,
2-(4-(1-((4-methyl-4H-1,2,4-triazol-3-yl)methyl)cyclobutyl)-6-((2-(2-oxopyrrolidin-1-yl)ethyl)amino)pyridin-2-yl)-6-(((1-methylcyclobutyl)amino)methyl)-4-(trifluoromethyl)isoindolin-1-one,
2-(4-(1-((4-methyl-4H-1,2,4-triazol-3-yl)methyl)cyclobutyl)-6-((piperidin-4-ylmethyl)amino)pyridin-2-yl)-6-(((1-methylcyclobutyl)amino)methyl)-4-(trifluoromethyl)isoindolin-1-one,
2-(4-(1-((4-methyl-4H-1,2,4-triazol-3-yl)methyl)cyclobutyl)-6-((2-morpholinoethyl)amino)pyridin-2-yl)-6-(((1-methylcyclobutyl)amino)methyl)-4-(trifluoromethyl)isoindolin-1-one,
2-(3-(3,3-difluoro-1-((4-methyl-4H-1,2,4-triazol-3-yl)methyl)cyclobutyl)phenyl)-6-(((3-fluoro-1-methylcyclobutyl)amino)methyl)-4-(trifluoromethyl)isoindolin-1-one,
2-(6-isobutyl-4-(1-((4-methyl-4H-1,2,4-triazol-3-yl)methyl)cyclobutyl)pyridin-2-yl)-6-(((1-methylcyclobutyl)amino)methyl)-4-(trifluoromethyl)isoindolin-1-one,
2-(4-(1-((4-methyl-4H-1,2,4-triazol-3-yl)methyl)cyclobutyl)-6-propylpyridin-2-yl)-6-(((1-methylcyclobutyl)amino)methyl)-4-(trifluoromethyl)isoindolin-1-one,
2-(3-(3,3-difluoro-1-((4-methyl-4H-1,2,4-triazol-3-yl)methyl)cyclobutyl)phenyl)-6-(((1-methylcyclopentyl)amino)methyl)-4-(trifluoromethyl)isoindolin-1-one,
2-(6-benzyl-4-(1-((4-methyl-4H-1,2,4-triazol-3-yl)methyl)cyclobutyl)pyridin-2-yl)-6-(((1-methylcyclobutyl)amino)methyl)-4-(trifluoromethyl)isoindolin-1-one,
2-(6-butyl-4-(1-((4-methyl-4H-1,2,4-triazol-3-yl)methyl)cyclobutyl)pyridin-2-yl)-6-(((1-methylcyclobutyl)amino)methyl)-4-(trifluoromethyl)isoindolin-1-one,
2-(3-(3-(difluoro(4-methyl-4H-1,2,4-triazol-3-yl)methyl)oxetan-3-yl)phenyl)-6-(((1-methylcyclobutyl)amino)methyl)-4-(trifluoromethyl)isoindolin-1-one,
2-(6-(isobutylamino)-4-(1-((4-methyl-4H-1,2,4-triazol-3-yl)methyl)cyclobutyl)pyridin-2-yl)-6-(((1-methylcyclobutyl)amino)methyl)-4-(trifluoromethyl)isoindolin-1-one,
2-(6-(ethylthio)-4-(1-((4-methyl-4H-1,2,4-triazol-3-yl)methyl)cyclobutyl)pyridin-2-yl)-6-(((1-methylcyclobutyl)amino)methyl)-4-(trifluoromethyl)isoindolin-1-one,
2-(4-(1-((4-methyl-4H-1,2,4-triazol-3-yl)methyl)cyclobutyl)-6-(naphthalen-2-yl)pyridin-2-yl)-6-(((1-methylcyclobutyl)amino)methyl)-4-(trifluoromethyl)isoindolin-1-one,
2-(6-chloro-4-(1-((4-methyl-4H-1,2,4-triazol-3-yl)methyl)cyclobutyl)pyridin-2-yl)-6-(((1-methylcyclobutyl)amino)methyl)-4-(trifluoromethyl)isoindolin-1-one,
2-(3-(3,3-difluoro-1-((4-methyl-4H-1,2,4-triazol-3-yl)methyl)cyclobutyl)phenyl)-6-((((1-methylcyclopropyl)methyl)amino)methyl)-4-(trifluoromethyl)isoindolin-1-one,
2-(3-(3,3-difluoro-1-((4-methyl-4H-1,2,4-triazol-3-yl)methyl)cyclobutyl)phenyl)-6-(((1,3-dimethylcyclobutyl)amino)methyl)-4-(trifluoromethyl)isoindolin-1-one,
2-(3-(3,3-difluoro-1-((4-methyl-4H-1,2,4-triazol-3-yl)methyl)cyclobutyl)phenyl)-6-(((3-methylbicyclo[1.1.1]pentan-1-yl)amino)methyl)-4-(trifluoromethyl)isoindolin-1-one,
2-(3-(3,3-difluoro-1-((4-methyl-4H-1,2,4-triazol-3-yl)methyl)cyclobutyl)phenyl)-4-(trifluoromethyl)-6-(((1,2,2-trimethylcyclobutyl)amino)methyl)isoindolin-1-one,
6-((bicyclo[1.1.1]pentan-1-ylamino)methyl)-2-(3-(3,3-difluoro-1-((4-methyl-4H-1,2,4-triazol-3-yl)methyl)cyclobutyl)phenyl)-4-(trifluoromethyl)isoindolin-1-one,
2-(3-(3,3-difluoro-1-((4-methyl-4H-1,2,4-triazol-3-yl)methyl)cyclobutyl)phenyl)-6-((((2,2-dimethyl-1,3-dioxolan-4-yl)methyl)amino)methyl)-4-(trifluoromethyl)isoindolin-1-one,
2-(3-(3,3-difluoro-1-((4-methyl-4H-1,2,4-triazol-3-yl)methyl)cyclobutyl)phenyl)-6-((((tetrahydrofuran-3-yl)methyl)amino)methyl)-4-(trifluoromethyl)isoindolin-1-one,
2-(6-ethoxy-4-(1-((4-methyl-4H-1,2,4-triazol-3-yl)methyl)cyclobutyl)pyridin-2-yl)-6-(((1-methylcyclobutyl)amino)methyl)-4-(trifluoromethyl)isoindolin-1-one,
2-(6-(benzyloxy)-4-(1-((4-methyl-4H-1,2,4-triazol-3-yl)methyl)cyclobutyl)pyridin-2-yl)-6-(((1-methylcyclobutyl)amino)methyl)-4-(trifluoromethyl)isoindolin-1-one,
2-(6-ethyl-4-(1-((4-methyl-4H-1,2,4-triazol-3-yl)methyl)cyclobutyl)pyridin-2-yl)-6-(((1-methylcyclobutyl)amino)methyl)-4-(trifluoromethyl)isoindolin-1-one,
2-(6-cyclopropyl-4-(1-((4-methyl-4H-1,2,4-triazol-3-yl)methyl)cyclobutyl)pyridin-2-yl)-6-(((1-methylcyclobutyl)amino)methyl)-4-(trifluoromethyl)isoindolin-1-one,
2-(3-(3,3-difluoro-1-((4-methyl-4H-1,2,4-triazol-3-yl)methyl)cyclobutyl)phenyl)-6-((neopentylamino)methyl)-4-(trifluoromethyl)isoindolin-1-one,
6-((cyclopentylamino)methyl)-2-(3-(3,3-difluoro-1-((4-methyl-4H-1,2,4-triazol-3-yl)methyl)cyclobutyl)phenyl)-4-(trifluoromethyl)isoindolin-1-one,
6-((2-oxaspiro[3.3]heptan-6-ylamino)methyl)-2-(3-(3,3-difluoro-1-((4-methyl-4H-1,2,4-triazol-3-yl)methyl)cyclobutyl)phenyl)-4-(trifluoromethyl)isoindolin-1-one,
2-(3-(3,3-difluoro-1-((4-methyl-4H-1,2,4-triazol-3-yl)methyl)cyclobutyl)phenyl)-6-(((1-(methoxymethyl)cyclopropyl)amino)methyl)-4-(trifluoromethyl)isoindolin-1-one,
2-(3-(3,3-difluoro-1-((4-methyl-4H-1,2,4-triazol-3-yl)methyl)cyclobutyl)phenyl)-6-(((oxetan-3-ylmethyl)amino)methyl)-4-(trifluoromethyl)isoindolin-1-one,
2-(3-(3,3-difluoro-1-((4-methyl-4H-1,2,4-triazol-3-yl)methyl)cyclobutyl)phenyl)-6-(((1-ethylcyclobutyl)amino)methyl)-4-(trifluoromethyl)isoindolin-1-one,
(R)-6-((sec-butylamino)methyl)-2-(3-(3,3-difluoro-1-((4-methyl-4H-1,2,4-triazol-3-yl)methyl)cyclobutyl)phenyl)-4-(trifluoromethyl)isoindolin-1-one,
(S)-6-((sec-butylamino)methyl)-2-(3-(3,3-difluoro-1-((4-methyl-4H-1,2,4-triazol-3-yl)methyl)cyclobutyl)phenyl)-4-(trifluoromethyl)isoindolin-1-one,
2-(3-(3,3-difluoro-1-((4-methyl-4H-1,2,4-triazol-3-yl)methyl)cyclobutyl)phenyl)-6-(((1-fluoro-2-methylpropan-2-yl)amino)methyl)-4-(trifluoromethyl)isoindolin-1-one,
2-(3-(3,3-difluoro-1-((4-methyl-4H-1,2,4-triazol-3-yl)methyl)cyclobutyl)phenyl)-6-(((2-methoxy-2-methylpropyl)amino)methyl)-4-(trifluoromethyl)isoindolin-1-one,
2-(6-((cyclopropylmethyl)amino)-4-(1-((4-methyl-4H-1,2,4-triazol-3-yl)methyl)cyclobutyl)pyridin-2-yl)-6-(((1-methylcyclobutyl)amino)methyl)-4-(trifluoromethyl)isoindolin-1-one,
2-(4-(1-((4-methyl-4H-1,2,4-triazol-3-yl)methyl)cyclobutyl)-6-((pyridin-4-ylmethyl)amino)pyridin-2-yl)-6-(((1-methylcyclobutyl)amino)methyl)-4-(trifluoromethyl)isoindolin-1-one,
2-(3-(3,3-difluoro-1-((4-methyl-4H-1,2,4-triazol-3-yl)methyl)cyclobutyl)phenyl)-6-((isopropylamino)methyl)-4-(trifluoromethyl)isoindolin-1-one,
2-(3-(3,3-difluoro-1-((4-methyl-4H-1,2,4-triazol-3-yl)methyl)cyclobutyl)phenyl)-6-((isopropyl(methyl)amino)methyl)-4-(trifluoromethyl)isoindolin-1-one,
(R)-2-(3-(3,3-difluoro-1-(fluoro(4-methyl-4H-1,2,4-triazol-3-yl)methyl)cyclobutyl)phenyl)-6-((isopropylamino)methyl)-4-(trifluoromethyl)isoindolin-1-one,
(S)-2-(3-(3,3-difluoro-1-(fluoro(4-methyl-4H-1,2,4-triazol-3-yl)methyl)cyclobutyl)phenyl)-6-((isopropylamino)methyl)-4-(trifluoromethyl)isoindolin-1-one,
(R)-2-(3-(3,3-difluoro-1-(fluoro(4-methyl-4H-1,2,4-triazol-3-yl)methyl)cyclobutyl)phenyl)-6-((propylamino)methyl)-4-(trifluoromethyl)isoindolin-1-one,
(S)-2-(3-(3,3-difluoro-1-(fluoro(4-methyl-4H-1,2,4-triazol-3-yl)methyl)cyclobutyl)phenyl)-6-((propylamino)methyl)-4-(trifluoromethyl)isoindolin-1-one,
(R)-2-(3-(3,3-difluoro-1-(fluoro(4-methyl-4H-1,2,4-triazol-3-yl)methyl)cyclobutyl)phenyl)-6-(((3-fluoropropyl)amino)methyl)-4-(trifluoromethyl)isoindolin-1-one,
(S)-2-(3-(3,3-difluoro-1-(fluoro(4-methyl-4H-1,2,4-triazol-3-yl)methyl)cyclobutyl)phenyl)-6-(((3-fluoropropyl)amino)methyl)-4-(trifluoromethyl)isoindolin-1-one,
(R)-6-(((cyclopropylmethyl)amino)methyl)-2-(3-(3,3-difluoro-1-(fluoro(4-methyl-4H-1,2,4-triazol-3-yl)methyl)cyclobutyl)phenyl)-4-(trifluoromethyl)isoindolin-1-one,
(S)-6-(((cyclopropylmethyl)amino)methyl)-2-(3-(3,3-difluoro-1-(fluoro(4-methyl-4H-1,2,4-triazol-3-yl)methyl)cyclobutyl)phenyl)-4-(trifluoromethyl)isoindolin-1-one,
(R)-2-(3-(3,3-difluoro-1-(fluoro(4-methyl-4H-1,2,4-triazol-3-yl)methyl)cyclobutyl)phenyl)-6-((isobutylamino)methyl)-4-(trifluoromethyl)isoindolin-1-one,
(S)-2-(3-(3,3-difluoro-1-(fluoro(4-methyl-4H-1,2,4-triazol-3-yl)methyl)cyclobutyl)phenyl)-6-((isobutylamino)methyl)-4-(trifluoromethyl)isoindolin-1-one,
2-(3-(isopropylamino)-5-(1-((4-methyl-4H-1,2,4-triazol-3-yl)methyl)cyclobutyl)phenyl)-6-((propylamino)methyl)-4-(trifluoromethyl)isoindolin-1-one,
6-(((cyclopropylmethyl)amino)methyl)-2-(3-(ethylamino)-5-(1-((4-methyl-4H-1,2,4-triazol-3-yl)methyl)cyclobutyl)phenyl)-4-(trifluoromethyl)isoindolin-1-one,
2-(3-(ethylamino)-5-(1-((4-methyl-4H-1,2,4-triazol-3-yl)methyl)cyclobutyl)phenyl)-6-((propylamino)methyl)-4-(trifluoromethyl)isoindolin-1-one,
6-(((cyclopropylmethyl)amino)methyl)-2-(3-(isopropylamino)-5-(1-((4-methyl-4H-1,2,4-triazol-3-yl)methyl)cyclobutyl)phenyl)-4-(trifluoromethyl)isoindolin-1-one,
2-(3-(isopropylamino)-5-(1-((4-methyl-4H-1,2,4-triazol-3-yl)methyl)cyclobutyl)phenyl)-6-(((1-methylcyclobutyl)amino)methyl)-4-(trifluoromethyl)isoindolin-1-one,
2-(3-(ethylamino)-5-(1-((4-methyl-4H-1,2,4-triazol-3-yl)methyl)cyclobutyl)phenyl)-6-(((1-methylcyclobutyl)amino)methyl)-4-(trifluoromethyl)isoindolin-1-one,
(R)-2-(3-(3,3-difluoro-1-(fluoro(4-methyl-4H-1,2,4-triazol-3-yl)methyl)cyclobutyl)phenyl)-6-(((1-methylcyclobutyl)amino)methyl)-4-(trifluoromethyl)isoindolin-1-one,
(S)-2-(3-(3,3-difluoro-1-(fluoro(4-methyl-4H-1,2,4-triazol-3-yl)methyl)cyclobutyl)phenyl)-6-(((1-methylcyclobutyl)amino)methyl)-4-(trifluoromethyl)isoindolin-1-one,
2-(3-(3,3-difluoro-1-((4-methyl-4H-1,2,4-triazol-3-yl)methyl)cyclobutyl)-5-(ethylamino)phenyl)-6-(((1-methylcyclobutyl)amino)methyl)-4-(trifluoromethyl)isoindolin-1-one,
(Ir,3r)-3-((4-methyl-4H-1,2,4-triazol-3-yl)methyl)-3-(3-(6-(((1-methylcyclobutyl)amino)methyl)-1-oxo-4-(trifluoromethyl)isoindolin-2-yl)phenyl)cyclobutanecarbonitrile,
(Is,3s)-3-((4-methyl-4H-1,2,4-triazol-3-yl)methyl)-3-(3-(6-(((1-methylcyclobutyl)amino)methyl)-1-oxo-4-(trifluoromethyl)isoindolin-2-yl)phenyl)cyclobutanecarbonitrile,
(R)-4-chloro-2-(3-(3-(fluoro(4-methyl-4H-1,2,4-triazol-3-yl)methyl)oxetan-3-yl)phenyl)-6-(((1-methylcyclobutyl)amino)methyl)isoindolin-1-one,
(R)-4-bromo-2-(3-(3-(fluoro(4-methyl-4H-1,2,4-triazol-3-yl)methyl)oxetan-3-yl)phenyl)-6-(((1-methylcyclobutyl)amino)methyl)isoindolin-1-one,
2-(3-cyclopropyl-5-(3-((4-methyl-4H-1,2,4-triazol-3-yl)methyl)oxetan-3-yl)phenyl)-6-(((1-methylcyclobutyl)amino)methyl)-4-(trifluoromethyl)isoindolin-1-one,
2-(3-ethoxy-5-(3-((4-methyl-4H-1,2,4-triazol-3-yl)methyl)oxetan-3-yl)phenyl)-6-(((1-methylcyclobutyl)amino)methyl)-4-(trifluoromethyl)isoindolin-1-one,
2-(3-(ethylamino)-5-(3-((4-methyl-4H-1,2,4-triazol-3-yl)methyl)oxetan-3-yl)phenyl)-6-(((1-methylcyclobutyl)amino)methyl)-4-(trifluoromethyl)isoindolin-1-one,
2-(3-(cyclopentylamino)-5-(3,3-difluoro-1-((4-methyl-4H-1,2,4-triazol-3-yl)methyl)cyclobutyl)phenyl)-6-(((1-methylcyclobutyl)amino)methyl)-4-(trifluoromethyl)isoindolin-1-one,
2-(3-(cyclopentyloxy)-5-(3,3-difluoro-1-((4-methyl-4H-1,2,4-triazol-3-yl)methyl)cyclobutyl)phenyl)-6-(((1-methylcyclobutyl)amino)methyl)-4-(trifluoromethyl)isoindolin-1-one,
2-(3-(cyclopentyloxy)-5-(3-((4-methyl-4H-1,2,4-triazol-3-yl)methyl)oxetan-3-yl)phenyl)-6-(((1-methylcyclobutyl)amino)methyl)-4-(trifluoromethyl)isoindolin-1-one,
2-(3-(cyclopentylamino)-5-(3-((4-methyl-4H-1,2,4-triazol-3-yl)methyl)oxetan-3-yl)phenyl)-6-(((1-methylcyclobutyl)amino)methyl)-4-(trifluoromethyl)isoindolin-1-one,
2-(3-(1-((4-cyclopropyl-4H-1,2,4-triazol-3-yl)methyl)-3,3-difluorocyclobutyl)phenyl)-6-(((1-methylcyclobutyl)amino)methyl)-4-(trifluoromethyl)isoindolin-1-one, and
(R)-2-(3-(3-(fluoro(4-methyl-4H-1,2,4-triazol-3-yl)methyl)oxetan-3-yl)phenyl)-6-(((1-methylcyclobutyl)amino)methyl)-4-(trifluoromethyl)isoindolin-1-one;
or a pharmaceutically acceptable salt thereof.

107. A pharmaceutical composition comprising a compound, or a pharmaceutically acceptable salt thereof, according to claim 104, and one or more pharmaceutically acceptable excipients.

108. A method of treating a cancer, the method comprising administering to a subject in need thereof, a therapeutic amount of a compound according to claim 104.
